(19) United States
(12) Patent Application Publication
(10) Pub. No.: US 2016/0312289 A1
(43) Pub. Date: Oct. 27, 2016

(54) BIOMOLECULAR EVENTS IN CANCER REVEALED BY ATTRACTOR MOLECULAR SIGNATURES

(71) Applicant: THE TRUSTEES OF COLUMBIA UNIVERSITY IN THE CITY OF NEW YORK, New York, NY (US)

(72) Inventor: Dimitris Anastassiou, Tenafly, NJ (US)

(73) Assignee: THE TRUSTEES OF COLUMBIA UNIVERSITY IN THE CITY OF NEW YORK, New York, NY (US)

(21) Appl. No.: 14/952,065

(22) Filed: Nov. 25, 2015

Related U.S. Application Data
(63) Continuation of application No. PCT/US2014/031590, filed on Mar. 24, 2014.
(60) Provisional application No. 61/828,655, filed on May 29, 2013.

(51) Int. Cl. C12Q 1/68

(52) U.S. Cl. CPC C12Q 1/6886; C12Q 2600/178; C12Q 2600/118; C12Q 2600/106; C12Q 2600/154; C12Q 2600/158

(57) ABSTRACT

The present invention is directed to compositions and methods for the independent and unconstrained identification of attractor molecular signatures as surrogates of pure biomolecular events as well as the use of such attractor molecular in performing medical diagnosis, prognosis, and developing appropriate therapeutic regimes.

### CROSS-REFERENCE TO RELATED APPLICATIONS

The present application is a continuation of PCT Application Ser. No. PCT/US2014/031590, filed Mar. 24, 2014, which claims the benefit of U.S. Provisional Application Ser. No. 61/828,655, filed May 29, 2013, the disclosures of which are both incorporated by reference herein in their entirety.

### 1. BACKGROUND OF THE INVENTION

Rich datasets, such as the rich biomolecular datasets publicly available at an increasing rate from sources such as The Cancer Genome Atlas (TCGA), provide unique opportunities for discovery from purely computational analysis. For example, gene expression signatures resulting from analysis of cancer datasets can serve as surrogates of cancer phenotypes. (Nevins, J. R. & Potti, A. Nat Rev Genet 8, 601-609 (2007)). Subtypes in many cancer types (Collisson et al., Nat Med 17, 500-503 (2011); Verhaak et al., Cancer Cell 17, 98-110 (2010); and Cancer Genome Atlas Research, Nature 474, 609-615 (2011)) have been successfully identified by gene expression analysis often using techniques such as nonnegative matrix factorization (Brunet et al. Proc Natl Acad Sci USA 101, 4164-4169 (2004)) combined with consensus clustering. (Monti, et al., Machine Learning 52, 91-118 (2003)).

The main objective addressed by techniques such as nonnegative matrix factorization is to reduce dimensionality by identifying a number of metagenes jointly representing the gene expression dataset as accurately as possible, in lieu of the whole set of individual genes. Each metagene is defined as a positive linear combination of the individual genes, so that its expression level is an accordingly weighted average of the expression levels of the individual genes. The identity of each resulting metagene is influenced by the presence of other metagenes within the objective of overall dimensionality reduction achieved by joint optimization.

In contrast, if the aim is not dimensionality reduction or classification into subtypes, but instead the independent and unconstrained identification of metagenes or other molecular signatures (e.g., methylation state or protein expression) as surrogates of pure biomolecular events, then a different algorithm should be devised. This approach is devoid of cross-interference and has the advantage of increasing the chance of precisely identifying the few particular genes that are at the core of the underlying biological mechanism as those that have the highest weights in the corresponding metagene, thus shedding more light on that mechanism. The present invention relates to such an approach, including in the context of applications involving data sets other than those related to gene expression, as well as the molecular signatures identified thereby, and compositions & methods employing such molecular signatures.

### 2. SUMMARY OF THE INVENTION

In certain embodiments, the present invention is directed to compositions and methods for identifying an attractor from a data set, comprising: evaluating the data set, wherein the data set comprises information concerning a plurality of objects characterized by particular feature vectors and wherein the evaluation identifies, using a computer processor, an association between individual members of the plurality of objects; and selecting, from the plurality of objects, a set of two or more objects maximally associated with a composite version of the same set of objects, and thereby identifying an attractor from the data set.

In certain embodiments, the present invention is directed to compositions and methods for identifying an attractor molecular signature from a data set, comprising: evaluating the data set, wherein the data set comprises information relating to a plurality of genes, miRNA sequences, methylation states, and/or protein expression levels and wherein the evaluation identifies, using a computer processor, an association between individual members of the plurality of genes, miRNA sequences, methylation states, and/or protein expression levels; and selecting, from the plurality of genes, a set of two or more genes maximally associated with a composite version of the same set of genes, miRNA sequences, methylation states, and/or protein expression levels, and thereby identifying an attractor molecular signature from the data set.

In certain embodiments, the present invention is directed to compositions and methods for identifying an attractor molecular signature from a gene data set, comprising: evaluating the gene data set, wherein the gene data set comprises information from a plurality of genes and wherein the evaluation identifies, using a computer processor, an association between individual members of the plurality of genes; and selecting, from the plurality of genes, a set of two or more genes maximally associated with a composite version of the same set of genes, and thereby identifying an attractor metagene from the gene data set.

In certain embodiments of such methods, the composite version of the gene set comprising the attractor molecular signature, i.e., an attractor metagene, is a weighted average of the individual genes in which the weights are proportional to the associations of the corresponding individual genes with the metagene. In certain embodiments of such methods, said evaluation consists of an iterative process in which each iteration modifies a metagene defined as a weighted average of individual genes such that the weights become increasingly proportional to the associations of the corresponding individual genes with the metagene. In certain embodiments of such methods, the evaluation consists of an iterative process in which each iteration modifies a metagene comprising individual genes such that the individual genes are increasingly associated with a composite version of the same set of genes. In certain embodiments of such methods, the gene data set comprises expression levels for each of the plurality of genes. In certain embodiments of such methods, the gene data set comprises methylation values and/or protein expression level values for one or more of the plurality of genes.

In certain embodiments, the present invention is directed to a system for identifying an attractor molecular signature, e.g., an attractor metagene, from a data set, comprising: at least one processor and a computer readable medium coupled to the at least one processor, the computer readable medium having stored thereon instructions which when executed cause the processor to: evaluate the data set, wherein the data set comprises information from a plurality of genes and wherein the evaluation identifies, using the computer processor, an association between individual members of plurality of genes, miRNA sequences, methylation states, and/or protein expression levels; and selecting, from the plurality of genes, miRNA sequences, methylation states, and/or protein expression levels, a set of two or more genes, miRNA sequences, methylation states, and/or protein expression levels maximally associated with a composite version of the same set of genes, miRNA sequences, methylation states, and/or protein expression levels, and thereby identifying an attractor molecular signature from the data set.

In certain embodiments of such systems, the composite version of the data set comprising the attractor molecular signature is a weighted average of the individual genes, miRNA sequences, methylation states, and/or protein expression levels in which the weights are proportional to the associations of the corresponding individual genes, miRNA sequences, methylation states, and/or protein expression levels with the attractor molecular signnature. In certain embodiments of such systems, the evaluation consists of an iterative process in which each iteration modifies a molecular signature comprising individual genes, miRNA sequences, methylation states, and/or protein expression levels such that the individual genes, miRNA sequences, methylation states, and/or protein expression levels are increasingly associated with a composite version of the same set of genes, miRNA sequences, methylation states, and/or protein expression levels. In certain of such embodiments, the data set comprises expression levels for each of the plurality of genes, miRNA sequences, methylation states, and/or protein expression levels.

In certain embodiments, the present invention is directed to a kit for detecting the presence of an attractor molecular signature, such as, but not limited to an attractor metagene, comprising measuring means for one or more feature selected from the group consisting of the genes associated with an attractor molecular signature of FIGS. 3-18.

In certain embodiments, the present invention is directed to a kit for detecting the presence of an LYM mRNA attractor metagene comprising measuring means for one or more feature selected from the group consisting of the genes associated with the attractor metagene of FIG. 3 and FIG. 19.

In certain embodiments, the present invention is directed to a kit for detecting the presence of a CIN mRNA attractor metagene comprising measuring means for one or more feature selected from the group consisting of the genes associated with the attractor metagene of FIG. 4 and FIG. 19.

In certain embodiments, the present invention is directed to a kit for detecting the presence of an MES attractor metagene comprising measuring means for one or more feature selected from the group consisting of the genes associated with the attractor metagene of FIG. 5 and FIG. 19.

In certain embodiments, the present invention is directed to a kit for detecting the presence of an END attractor metagene comprising measuring means for one or more feature selected from the group consisting of the genes associated with the attractor metagene of FIG. 6 and FIG. 19.

In certain embodiments, the present invention is directed to a kit for detecting the presence of an AHSA2 mRNA attractor metagene comprising measuring means for one or more feature selected from the group consisting of the genes associated with the attractor metagene of FIG. 7 and FIG. 19.

In certain embodiments, the present invention is directed to a kit for detecting the presence of an IFIT mRNA attractor metagene comprising measuring means for one or more feature selected from the group consisting of the genes associated with the attractor metagene of FIG. 8 and FIG. 19.

In certain embodiments, the present invention is directed to a kit for detecting the presence of a WDR38 mRNA attractor metagene comprising measuring means for one or more feature selected from the group consisting of the genes associated with the attractor metagene of FIG. 9 and FIG. 19.

In certain embodiments, the present invention is directed to a kit for detecting the presence of a mir127 miRNA attractor molecular signature comprising measuring means for one or more feature selected from the group consisting of the genes associated with the attractor molecular signature of FIG. 10 and FIG. 19.

In certain embodiments, the present invention is directed to a kit for detecting the presence of a mir509 miRNA attractor molecular signature comprising measuring means for one or more feature selected from the group consisting of the genes associated with the attractor molecular signature of FIG. 11 and FIG. 19.

In certain embodiments, the present invention is directed to a kit for detecting the presence of a mir144 miRNA attractor molecular signature comprising measuring means for one or more feature selected from the group consisting of the genes associated with the attractor molecular signature of FIG. 12 and FIG. 19.

In certain embodiments, the present invention is directed to a kit for detecting the presence of a RMND1 methylation attractor molecular signature comprising measuring means for one or more feature selected from the group consisting of the methylation states associated with the attractor molecular signature of FIG. 13 and FIG. 19.

In certain embodiments, the present invention is directed to a kit for detecting the presence of a M+ methylation attractor molecular signature comprising measuring means for one or more feature selected from the group consisting of the methylation states associated with the attractor molecular signature of FIG. 14 and FIG. 19.

In certain embodiments, the present invention is directed to a kit for detecting the presence of a M− attractor molecular signature comprising measuring means for one or more feature selected from the group consisting of the methylaton states associated with the attractor molecular signature of FIG. 15 and FIG. 19.

In certain embodiments, the present invention is directed to a kit for detecting the presence of a c-MET protein attractor molecular signature comprising measuring means for one or more feature selected from the group consisting of the protein expression levels associated with the attractor molecular signature of FIG. 16 and FIG. 19.

In certain embodiments, the present invention is directed to a kit for detecting the presence of a Akt protein attractor molecular signature comprising measuring means for one or more feature selected from the group consisting of the protein expression levels associated with the attractor molecular signature of FIG. 17 and FIG. 19.

In certain of the foregoing embodiments relating to kits, the present invention is also directed to kits that further comprise a control sample.

In certain embodiments, the present invention is directed to a method of treatment wherein a patient sample is assayed for the presence of one or more feature selected from the group consisting of the genes associated with an LYM mRNA attractor metagene of FIG. 3 and FIG. 19 and wherein, if the feature associated with the attractor metagene is present, thereafter adjusting the treatment accordingly.

In certain embodiments, the present invention is directed to a method of treatment wherein a patient sample is assayed for the presence of one or more feature selected from the group consisting of the genes associated with the CIN mRNA attractor metagene of FIG. 4 and FIG. 19 and wherein, if the feature associated with the attractor metagene is present, thereafter adjusting the treatment accordingly.

In certain embodiments, the present invention is directed to a method of treatment wherein a patient sample is assayed for the presence of one or more feature selected from the group consisting of the genes associated with the MES mRNA attractor metagene of FIG. 5 and FIG. 19 and wherein, if the feature associated with the attractor metagene is present, thereafter adjusting the treatment accordingly.

In certain embodiments, the present invention is directed to a method of treatment wherein a patient sample is assayed for the presence of one or more feature selected from the group consisting of the genes associated with the END mRNA attractor metagene of FIG. 6 and FIG. 19 and wherein, if the feature associated with the attractor metagene is present, thereafter adjusting the treatment accordingly.

In certain embodiments, the present invention is directed to a method of treatment wherein a patient sample is assayed for the presence of one or more feature selected from the group consisting of the genes associated with the ASHA2 mRNA attractor metagene of FIG. 7 and FIG. 19 and wherein, if the feature associated with the attractor metagene is present, thereafter adjusting the treatment accordingly.

In certain embodiments, the present invention is directed to a method of treatment wherein a patient sample is assayed for the presence of one or more feature selected from the group consisting of the genes associated with the IFIT mRNA attractor metagene of FIG. 8 and FIG. 19 and wherein, if the feature associated with the attractor metagene is present, thereafter adjusting the treatment accordingly.

In certain embodiments, the present invention is directed to a method of treatment wherein a patient sample is assayed for the presence of one or more feature selected from the group consisting of the genes associated with the WDR38 mRNA attractor metagene of FIG. 9 and FIG. 19 and wherein, if the feature associated with the attractor metagene is present, thereafter adjusting the treatment accordingly.

In certain embodiments, the present invention is directed to a method of treatment wherein a patient sample is assayed for the presence of one or more feature selected from the group consisting of the genes associated with the mir127 miRNA attractor molecular signature of FIG. 10 and FIG. 19 and wherein, if the feature associated with the attractor molecular signature is present, thereafter adjusting the treatment accordingly.

In certain embodiments, the present invention is directed to a method of treatment wherein a patient sample is assayed for the presence of one or more feature selected from the group consisting of the genes associated with the mir509 miRNA attractor molecular signature of FIG. 11 and FIG. 19 and wherein, if the feature associated with the attractor molecular signature is present, thereafter adjusting the treatment accordingly.

In certain embodiments, the present invention is directed to a method of treatment wherein a patient sample is assayed for the presence of one or more feature selected from the group consisting of the genes associated with the mir144 miRNA attractor molecular signature of FIG. 12 and FIG. 19 and wherein, if the feature associated with the attractor molecular signature is present, thereafter adjusting the treatment accordingly.

In certain embodiments, the present invention is directed to a method of treatment wherein a patient sample is assayed for the presence of one or more feature selected from the group consisting of the methylation states associated with the RMND1 methylation attractor molecular signature of FIG. 13 and FIG. 19 and wherein, if the feature associated with the attractor molecular signature is present, thereafter adjusting the treatment accordingly.

In certain embodiments, the present invention is directed to a method of treatment wherein a patient sample is assayed for the presence of one or more feature selected from the group consisting of the methylation states associated with the M+ methylation attractor molecular signature of FIG. 14 and FIG. 19 and wherein, if the feature associated with the attractor molecular signature is present, thereafter adjusting the treatment accordingly.

In certain embodiments, the present invention is directed to a method of treatment wherein a patient sample is assayed for the presence of one or more feature selected from the group consisting of the methylation states associated with the M− methylation attractor molecular signature of FIG. 15 and FIG. 19 and wherein, if the feature associated with the attractor molecular signature is present, thereafter adjusting the treatment accordingly.

In certain embodiments, the present invention is directed to a method of treatment wherein a patient sample is assayed for the presence of one or more feature selected from the group consisting of the protein expression levels associated with the c-MET protein attractor molecular signature of FIG. 16 and FIG. 19 and wherein, if the feature associated with the attractor molecular signature is present, thereafter adjusting the treatment accordingly.

In certain embodiments, the present invention is directed to a method of treatment wherein a patient sample is assayed for the presence of one or more feature selected from the group consisting of the protein expression levels associated with the Akt protein attractor molecular signature of FIG. 17 and FIG. 19 and wherein, if the feature associated with the attractor molecular signature is present, thereafter adjusting the treatment accordingly.

In certain embodiments, the present invention provides for methods of performing a prognosis of a subject identified as having cancer, such as, but not limited to, methods comprising performance of a diagnostic method as set forth herein (e.g., obtaining a sample from the subject and determining whether an attractor molecular signature can be detected in the sample) and then, if an attractor molecular signature is detected in a sample of the subject, predicting the likely outcome (i.e., performing a prognosis) of the cancer, e.g., the likely survival duration. In certain embodiments, the prognosis will be based on the presence of one or more attractor molecular signature. In certain embodiments, the prognosis will be based on the presence of one or more attractor molecular signature and one or more additional factors, such as clinical and molecular features (e.g., the number of cancer-positive lymph nodes, age at diagnosis, and expression levels of particular genes exhibiting protective activity).

### 3. BRIEF DESCRIPTION OF THE FIGURES

FIGS. 1A-D depicts scatter plots of three genes from twelve cancer types. Each dot represents a cancer sample. The horizontal and vertical axes measure the expression values of two of the three genes, while the value of the third gene is color-coded. The observed linear change from lower left (blue) to upper right (red) demonstrates the coexpression of these three genes. Shown are scatter plots for the top-ranked three genes of (A) the CIN metagene, (B) the MES metagene, (C) the LYM metagene and (D) the END metagene.

FIG. 2 depicts scatter plots connecting the LYM, M+ and M− molecular signatures in 12 cancer types. Each dot represents a cancer sample. The horizontal and vertical axes measure the average methylation values of the two methylation signatures, M− and M+, while the value of the expression of the LYM metagene is color-coded. In all three cases, the molecular signature is defined by the average of the corresponding top ten genes/methylation states.

FIG. 3 depicts scatter plots of the top three features for the LYM mRNA attractor metagene. Each dot represents a cancer sample. The horizontal and vertical axes measure the values of two of the three features, while the value of the third feature is color-coded from blue to red.

FIG. 4 depicts scatter plots of the top three features for the CIN mRNA attractor metagene. Each dot represents a cancer sample. The horizontal and vertical axes measure the values of two of the three features, while the value of the third feature is color-coded from blue to red.

FIG. 5 depicts scatter plots of the top three features for the MES mRNA attractor metagene. Each dot represents a cancer sample. The horizontal and vertical axes measure the values of two of the three features, while the value of the third feature is color-coded from blue to red.

FIG. 6 depicts scatter plots of the top three features for the END mRNA attractor metagene. Each dot represents a cancer sample. The horizontal and vertical axes measure the values of two of the three features, while the value of the third feature is color-coded from blue to red.

FIG. 7 depicts scatter plots of the top three features for the AHSA2 mRNA attractor metagene. Each dot represents a cancer sample. The horizontal and vertical axes measure the values of two of the three features, while the value of the third feature is color-coded from blue to red.

FIG. 8 depicts scatter plots of the top three features for the IFIT mRNA attractor metagene. Each dot represents a cancer sample. The horizontal and vertical axes measure the values of two of the three features, while the value of the third feature is color-coded from blue to red.

FIG. 9 depicts scatter plots of the top three features for the WDR38 mRNA attractor metagene. Each dot represents a cancer sample. The horizontal and vertical axes measure the values of two of the three features, while the value of the third feature is color-coded from blue to red.

FIG. 10 depicts scatter plots of the top three features for the mir127 miRNA attractor molecular signature. Each dot represents a cancer sample. The horizontal and vertical axes measure the values of two of the three features, while the value of the third feature is color-coded from blue to red.

FIG. 11 depicts scatter plots of the top three features for the mir509 miRNA attractor molecular signature. Each dot represents a cancer sample. The horizontal and vertical axes measure the values of two of the three features, while the value of the third feature is color-coded from blue to red.

FIG. 12 depicts scatter plots of the top three features for the mir144 miRNA attractor molecular signature. Each dot represents a cancer sample. The horizontal and vertical axes measure the values of two of the three features, while the value of the third feature is color-coded from blue to red.

FIG. 13 depicts scatter plots of the top three features for the RMND1 methylation attractor molecular signature. Each dot represents a cancer sample. The horizontal and vertical axes measure the values of two of the three features, while the value of the third feature is color-coded from blue to red.

FIG. 14 depicts scatter plots of the top three features for the M+ methylation attractor molecular signature. Each dot represents a cancer sample. The horizontal and vertical axes measure the values of two of the three features, while the value of the third feature is color-coded from blue to red.

FIG. 15 depicts scatter plots of the top three features for the M− methylation attractor molecular signature. Each dot represents a cancer sample. The horizontal and vertical axes measure the values of two of the three features, while the value of the third feature is color-coded from blue to red.

FIG. 16 depicts scatter plots of the top three features for the c-Met protein attractor molecular signature. Each dot represents a cancer sample. The horizontal and vertical axes measure the values of two of the three features, while the value of the third feature is color-coded from blue to red.

FIG. 17 depicts scatter plots of the top three features for the Akt protein attractor molecular signature. Each dot represents a cancer sample. The horizontal and vertical axes measure the values of two of the three features, while the value of the third feature is color-coded from blue to red.

FIG. 18 depicts scatter plots demonstrating the association between MES and END attractor molecular signature. The horizontal and vertical axes measure the values of the MES and END signatures. The two signatures have positive correlation, although this association is not sufficiently strong to merge the two attractors into one. This association suggests that the invasive MES signature and the antiangiogenic END signature tend to be present simultaneously

FIGS. 19A-D depicts molecular signatures in individual cancer types, shown as attractor clusters: (A) mRNA, (B) miRNA, (C) DNA methylation and (D) protein, containing seven, three, three and two signatures respectively, for a total of 15 molecular signatures. Attractor clusters are separated by two empty rows. Each row in the attractor cluster contains the top features of an attractor, as described in the Materials & Methods section, in the Example 1 section below. The first column includes the IDs of attractors, which indicates the cancer type in which it was found. The last column gives the strengths of each attractor, as described in the Methods & Materials section, in the Example 1 section below. The last row of each attractor cluster gives the top overlapping features in the attractor cluster and the number of cancer types in which the features were found in the attractor.

FIGS. 20A-D depicts consensus rankings of features in each molecular signature: (A) mRNA, (B) miRNA, (C) DNA methylation and (D) protein, containing seven, three, three and two signatures respectively, for a total of 15 molecular signatures. Each signature is represented by two columns, the first of which contains the list of features and the second contains, for each feature, the corresponding score, defined as the mutual information with the converged attractor with a cutoff score of 0.5.

FIG. 21 depicts genomically localized molecular signatures (shown as attractor clusters) in individual cancer types, including their chromosomal locations: mRNA, miRNA, DNA methylation and protein. An mRNA attractor cluster containing only genes on the Y chromosome was removed, because its selection was gender-based.

FIGS. 22A-B depicts Kaplan-Meier survival curves on the basis of (A) the FGD3-SUSD3 metagene (B) the ESR1 gene, in five data sets. P values were derived using the log-rank test after dividing each data set into two equal-sized subgroups.

FIG. 23 depicts Breast cancer specific 10-year survival rate as a function of the BCAM score normalized as the percentile value against the 1,981-sample METABRIC data set.

### 4. DETAILED DESCRIPTION OF THE INVENTION

The present invention is directed to compositions and methods for the independent and unconstrained identification of attractors out of rich datasets. In certain embodiments, the present invention is directed, in part, to compositions and methods for the independent and unconstrained identification of molecular signatures as surrogates of pure biomolecular events. For example, given a rich dataset represented by a gene, miRNA sequence, methylation state, and/or protein expression level matrix, such surrogate molecular signatures can be naturally identified as stable and precise attractors using a simple iterative approach. The identification processes of the present invention can be totally unsupervised, as the processes need not make use of any phenotypic association. Once identified, however, an attractor molecular signature is likely to be found associated with a phenotype. This approach is devoid of cross-interference and has the advantage of increasing the chance of precisely identifying the few particular features (e.g., gene, miRNA sequence, methylation state, and/or protein expression level) that are at the core of the underlying biological mechanism as those that have the highest weights in the corresponding molecular signature, thus shedding more light on that mechanism.

In certain embodiments, attractor metagenes have been identified as present in nearly identical form in multiple cancer types. This provides an additional opportunity to combine the powers of a large number of rich datasets to focus, at an even sharper level, on the core genes of the underlying mechanism. For example, this methodology can precisely point to the causal (driver) oncogenes within amplicons to be among very few candidate genes. This can be done from rich data sets, which already exist in abundance, without the requirement of generating and/or using sequencing data.

For clarity and not by way of limitation, this detailed description is divided into the following sub-portions:
4.1. Identification of Attractor Molecular Signatures;
4.2. Attractor Molecular Signatures Identified in Pancanl2 Data Set
4.3. Diagnosis & Treatment Employing Attractor Molecular Signatures

### 4.1. Identification of Attractor Molecular Signatures

### 4.1.1. Introduction to Attractor Metagenes

The instant application is directed, in part, to the identification and use of “attractor molecular signatures.” Although described in connection with data sets relating to genes, miRNA sequences, methylation states, and/or protein expression levels, the techniques described herein for identifying attractors find significantly broader use than solely in connection with such data. For example, but not by way of limitation, the algorithms described herein can be used for identifying attractor molecular signatures present in virtually any rich dataset, whether it relates to gene expression data, physiological activity (e.g., neuronal activity), or even commercial data (e.g., purchasing patterns or the use of social media). Thus, while the identification of genes will be employed as one example of the algorithms disclosed herein, the scope of the instant application is not so limited and can be implemented to identify objects characterized by any type of feature vectors.

Given a nonnegative measure J(Gᵢ, Gⱼ) of pairwise association between genes Gⁱ and Gⱼ, an attractor metagene can be defined as

$$M = {\sum }_{i} {w}_{i} {G}_{i}$$

to be a linear combination of the individual genes with weights wᵢ=J(Gᵢ, M). The association measure J is assumed to have minimum possible value 0 and maximum possible value 1, so the same is true for the weights. It is also assumed to be scale-invariant, therefore it is not necessary for the weights to be normalized so that they add to 1, and the metagenes can still be thought of as expressing a normalized weighted average of the expression levels of the individual genes, miRNA sequences, methylation states, and/or protein expression levels.

According to this definition, the genes with the highest weights in an attractor molecular signature will have the highest association with the molecular signature (and, by implication, they will tend to be highly associated among themselves) and so they will often represent a biomolecular event reflected by the co-expression of these top genes, miRNA sequences, methylation states, and/or protein expression levels. This can happen, e.g., when a biological mechanism is activated, or when a copy number variation (CNV), such as an amplicon, is present, in some of the samples included in the expression matrix.

As used herein, the term “attractor molecular signature,” means a signature of, e.g., coexpressed genes, miRNA sequences, methylation states, and/or protein expression levels. The phrase “top genes” or “attractor metagene” refers to the genes with the highest weights in a particular attractor molecular signature consisting of data relating to gene expression. As noted above,=, in certain embodiments, the definition of an attractor molecular signature can readily be generalized to include features other than gene expression, such as, but not limited to, methylation states or protein expression levels. In certain embodiments, the term attractor can be used in datasets of any objects (not necessarily genes) characterized by any type of feature vectors.

The computational problem of identifying attractor molecular signatures given an expression matrix can be addressed heuristically using a simple iterative process: Starting from a particular seed (or “attractee”) molecular signature M, a new molecular signature is defined in which the new weights are wᵢ=J(Gᵢ, M). The same process is then repeated in the next iteration resulting in a new set of weights, and so forth. Given a sufficient number of iterations, such a process will converge to a limited number of stable attractors. Each attractor is defined by a precise set of weights, which are reached with high accuracy, and, in certain embodiments, within 10 or 20 iterations.

This algorithmic behavior with convergence properties occurs due to the fact that if a molecular signature contains some co-expressed genes (or other features) with high weights, then the next iteration will naturally “attract” even more genes (or other features) with the same properties, and so forth, until the process will eventually converge to a molecular signature representing a potential underlying biological event reflected by this co-expression. Therefore, in certain embodiments, this methodology provides an unsupervised algorithm of identifying biomolecular events from rich biological data. Furthermore, in certain embodiments, the set of the few genes (or other features) with the highest weight can represent the “heart” (core) of the biomolecular event. In support of this concept, the association of any of the top-ranked individual genes (or other features) with the attractor molecular signature is consistently and significantly higher than the pairwise association between any of these features, suggesting that, in certain embodiments, the set of these top genes (or other features) are synergistically associated, comprising a proxy representing a biomolecular event in a better way than each of the individual features would. In certain embodiments, these proxy attractor molecular signatures can then be used within the context of Bayesian methods to identify regulatory interactions in a more straightforward manner than having to jointly identify clusters of co-expressed genes (or other features) and regulatory interactions.

Indeed, in certain instances, particular aspects of attractors identified using the techniques described herein have been previously identified in various contexts, often intermingled with additional genes or other features that may be unrelated or weakly related with the actual underlying mechanism. The techniques described herein, however, allow for recognition of certain attractors as multi-cancer biomolecular events and their composition is “purified” as a result of the attractor convergence to represent the core of the mechanism. Therefore the top features of the attractors will be most appropriate to be used as biomarkers or for improved understanding of the underlying biology and for identifying potential therapeutic targets. For example, certain aspects related to the mitotic CIN attractor descried herein have been previously described generally (Whitfield et al., Nat Rev Cancer 6, 99-106 (2006)) as “proliferation” or “cell-cycle related” markers, while the actual attractor, identified for the first time herein, points much more sharply to particular elements in the kinetochore structure.

In certain embodiments, a reasonable implementation of an “exhaustive” search will include only consider the seed molecular signatures in which one selected “attractee” feature is assigned a weight of 1 and all the other features are assigned a weight of 0. The molecular feature resulting from the next iteration will then assign high weights to all genes highly associated with the originally selected feature, referred to as the “attractee feature.” For example, if the feature is a gene, then the attractee feature will be an attractee gene. In this way all attractors representing biomolecular events characterized by coordinated features will be identified when these features are used as attractees. A computational implementation of an algorithm associated to such an embodiment is described herein. In certain embodiments, a dual method can be used to identify attractor “metasamples” as representatives of subtypes, and in certain embodiments such metasamples can be combined with the attractor molecular signatures in various ways to achieve biclustering.

### 4.1.2. General Attractor Finding Algorithm

As noted above, while the instant application describes the identification of attractors in the context of biological information, the general attractor finding algorithm described herein can be applied to virtually any rich data set, regardless of the particular nature of the data. Accordingly, while the instant application will describe the use of algorithms in the particular context of identifying attractor molecular signatures, it is understood that alternative attractors, depending the nature of the data set, can be identified. Thus, in the context of identifying attractor molecular signatures the association measure J(Gᵢ, Gⱼ) between genes (which in other contexts would represent the association measure between two alternative features) is selected to be a power function with exponent a of a normalized estimated information theoretic measure of the mutual information J(Gᵢ, Gⱼ) with minimum value 0 and maximum value 1, as a proper compromise between performance and complexity (although more sophisticated related association measures can also be used). (Cover, T. M. & Thomas, J. A. Elements of information theory, Edn. 2nd. Wiley-Interscience, Hoboken, N.J.; (2006); and Reshef et al., Science 334, 1518-1524 (2011)). In other words, J(Gᵢ, Gⱼ)=J^{a}(Gᵢ, Gⱼ) in which the exponent a can be any nonnegative number. As described in Examples section, each iteration of the algorithm will define a new molecular signature in which the weight wᵢ for gene Gᵢ will be equal to wᵢ=J(Gᵢ, M), where M is the immediately preceding molecular signature. The process is repeated until the magnitude of the difference between two consecutive weight vectors is less than a threshold, which can be selected, in certain embodiments, to be equal to 10⁻⁷.

In certain embodiments, algorithms useful in the context of the present invention can be described in simple MATLAB computer language as follows:

when given a gene expression matrix “E” of size ngenes x nsamples, where “ngenes” is the number of genes and “nsamples” is the number of samples. The single-row vector “weights” has size ngenes and contains the corresponding weights of a metagene. In each iteration, the molecular signature, which in this example is a metagene, being the weighted average of the expression values of the individual genes, is modified according to the following MATLAB code, in which “association” is an association measure function between two genes defined by their expression values:

for j=1:nsamples
metagene (j)=weights*E(:,j);

end

for i=1:ngenes
weights(i)=association(E(i), metagene)

end.

Alternatively, the attractor finding algorithm can identify unweighted “attractor gene sets” of size “attractorsize,” which can be fixed or adaptively varying. In that case, if the indices of the rows of the member genes are defined by a vector named “members,” then the metagene will be the simple average of the member genes. Each iteration leads to a new gene set consisting of the new set of top-ranked genes in terms of their association with the previous metagene. Therefore, in each iteration, the metagene will be modified as follows:

metagene=mean(E(members,:),1);

for i=1:ngenes
vect(i)=as sociation(E(i), metagene);

end

[Y I]=sort(vect, ‘descend’);

members=I(1:attractorsize).

In certain embodiments, the result of the instant process is tunable in terms of a parameter of “sharpness” of the attractor. This sharpness is based on a nonlinear function “f” of a known original association function “I” like the mutual information or the Pearson coefficient. Thus, in certain embodiments, the final “association function J” used to fit the definition of attractor can be f(I)=I^{a}, where the range of the continuously varying exponent “a” can be from zero to infinity. In certain non-limiting embodiments, “a” will be a large number, e.g., 10-10¹⁰ or a very small number, e.g., from about 0.5 to 10⁻¹⁰. At one extreme, if “a” is very large then each of the seeds will create its own single-gene attractor because all other genes will always have near-zero weights. In such embodiments, the total number of attractors will be equal to the number of genes. At the other extreme, if “a” is zero then all weights will remain equal to each other, thus representing the average of all genes (or other features), so there will only be one attractor. The higher the value of “a,” the “sharper” (more focused on its top gene) each attractor will be and the higher the overall number of attractors will be. As the value of “a” is gradually decreased, the attractor from a particular seed will transform itself, and in certain embodiments in a discontinuous manner, thus providing insight into potential related biological mechanisms.

In certain embodiments, an appropriate choice of “a” (in the sense of revealing single biomolecular events of coordinated features) for general attractors is around is from about 0.5 to about 10, in certain embodiments from 1 to about 6, and in certain embodiments a is about 5. In embodiments where a is about 5, there will typically be approximately 50 to 150 resulting attractors, each resulting from numerous attractee features, depending on the number of features and the cancer type. (An alternative to the power function can be a sigmoid function with varying steepness, but the consistency of the resulting attractors can, in certain embodiments, be decreased as compared to other techniques).

In certain embodiments, an attractor molecular signature can also be interpreted as a set of coordinated features containing a number among the top features of the attractor. In such cases, one can define the size of such set so that the set contains only the features that are significantly associated with the attractor. One such empirical criterion would be to include the features whose z-score of their mutual information with the attractor exceeds a large threshold, such as, but not limited to, exceeding a z-score of 20.

Identified attractors can be ranked in various ways. In certain embodiments, the “strength of an attractor” will be defined as the mutual information between the n^{th} top gene of the attractor and the attractor molecular signature itself. Indeed, if this measure is high, this implies that at least the top n features of the attractor are strongly coordinated. In certain embodiments, n=50 can be selected as a reasonable choice, not too large, but sufficiently so to represent a real complex biological phenomenon of coordination of at least 50 features. For amplicons, n=5 is sufficient to ensure that, e.g., the oncogenes are included in coordinated co-expression).

### 4.2. Attractor Molecular Signatures Identified in Pancanl2 Data Set

### 4.2.1. A Mesenchymal Transition Attractor Metagene

This attractor contains mostly epithelial-mesenchymal transition (EMT)-associated genes. This is a stage-associated attractor, in which the signature is significantly present only when a particular level of invasive stage, specific to each cancer type, has been reached. This phenomenon is observed, in three cancer datasets from different types (breast, ovarian and colon) that were annotated with clinical staging information, by providing a listing of differentially expressed genes, ranked by fold change, when ductal carcinoma in situ (DCIS) progresses to invasive ductal carcinoma; colon cancer progresses to stage II; and ovarian cancer progresses to stage III. In all three cases, the attractor is highly enriched among the top genes.

This attractor has been previously identified with remarkable accuracy as representing a particular kind of mesenchymal transition of cancer cells present in all types of solid cancers tested leading to a published list of top 64 genes. (Kim et al., BMC Med Genomics 3, 51 (2010); and Anastassiou et al., BMC Cancer 11, 529 (2011)). Most of the genes of the signature were found to be expressed by the cancer cells themselves, and not by the surrounding stroma, at least in a neuroblastoma xenograft model. (Anastassiou et al., BMC Cancer 11, 529 (2011)). The signature is found to be associated with prolonged time to recurrence in glioblastoma. (Cheng et al., PLoS One 7, e34705 (2012). Related versions of the same signature were previously found to be associated with resistance to neoadjuvant therapy in breast cancer. (Farmer et al., Nat Med 15, 68-74 (2009)). These results are consistent with the finding that EMT induces cancer cells to acquire stem cell properties. (Mani et al., Cell 133, 704-715 (2008)). It has been hypothesized that EMT is a key mechanism for cancer cell invasiveness and motility. (Hay, Acta Anat (Basel) 154, 8-20 (1995); Thiery, Nat Rev Cancer 2, 442-454 (2002); and Kalluri et al., J Clin Invest 119, 1420-1428 (2009)). The attractor, however, appears to represent a more general phenomenon of transdifferentiation present even in nonepithelial cancers such as neuroblastoma, glioblastoma and Ewing's sarcoma.

Although similar signatures are often labeled as “stromal,” because they contain many stromal markers such as α-SMA and fibroblast activation protein, the fact that most of the genes of the signature were expressed by xenografted cancer cells (Anastassiou et al., BMC Cancer 11, 529 (2011)), and not by mouse stromal cells, suggests that this particular attractor of coordinately expressed genes represents cancer cells having undergone a mesenchymal transition. The signature may indicate a non-fibroblastic transition, as occurs in glioblastoma, in which case collagen COL11A1 is not co-expressed with the other genes of the attractor. It is believed that a full fibroblastic transition of the cancer cells occurs when cancer cells encounter adipocytes (Anastassiou et al., BMC Cancer 11, 529 (2011)), in which case they may well assume the duties of cancer associated fibroblasts (CAFs) in some tumors. Hanahan et al., Cell 144, 646-674 (2011)). In that case, the best proxy of the signature (Kim et al., BMC Med Genomics 3, 51 (2010)) is COL11A1 and the strongly co-expressed genes THBS2 and INHBA. Indeed, the 64 genes of the previously identified signature were found from multi-cancer analysis (Kim et al., BMC Med Genomics 3, 51 (2010)) as the genes whose expression is consistently most associated with that of COL11A1.

The only EMT-inducing transcription factor found upregulated in the xenograft model (Anastassiou et al., BMC Cancer 11, 529 (2011)) is SNAI2 (Slug), and it is also the one most associated with the signature in publicly available datasets. The microRNAs found to be most highly associated with this attractor are miR 214, miR 199a, and miR-199b. Interestingly, miR-214 and miR-199a were found to be jointly regulated by another EMT-inducing transcription factor, TWIST1 (Yin et al., Oncogene 29, 3545-3553 (2010)).

### 4.2.2. A Mitotic CIN Attractor Metagene

This attractor contains mostly kinetochore-associated genes. Contrary to the stage associated mesenchymal transition attractor, this is a grade associated attractor, in which the signature is significantly present only when an intermediate level of tumor grade is reached. This phenomenon can be observed, in three cancer datasets from different types (breast, ovarian and bladder) that were annotated with tumor grade information, by providing a listing of differentially expressed genes, ranked by fold change, when grade G2 is reached. In all three cases, the attractor is highly enriched among the top genes. Consistently, a similar “gene expression grade index” signature was previously found differentially expressed between histologic grade 3 and histologic grade 1 breast cancer samples. (Sotiriou et al., Journal of the National Cancer Institute 98, 262-272 (2006)). Furthermore, that same signature was found capable of reclassifying patients with histologic grade 2 tumors into two groups with high versus low risks of recurrence. (Sotiriou et al., Journal of the National Cancer Institute 98, 262-272 (2006)).

This attractor is associated with chromosomal instability (CIN), as evidenced from the fact that another similar gene set comprising a “signature of chromosomal instability” (Carter et al., Nat Genet 38, 1043-1048 (2006)) was previously derived from multiple cancer datasets purely by identifying the genes that are most correlated with a measure of aneuploidy in tumor samples. This led to a 70-gene signature referred to as “CIN70.” However, several top genes of the attractor, such as CENPA, KIF2C, BUB 1 and CCNA2 are not present in the CIN70 list. Mitotic CIN is increasingly recognized as a widespread multi-cancer phenomenon. (Schvartzman, J. M., Sotillo, R. & Benezra, R. Mitotic chromosomal instability and cancer: mouse modelling of the human disease. Nat Rev Cancer 10, 102-115 (2010)).

The attractor is characterized by overexpression of kinetochore-associated genes, which are known (Yuen et al., Current Opinion in Cell Biology 17, 576-582 (2005)) to induce chromosomal instability (CIN) for reasons that are not clear. Overexpression of several of the genes of the attractor, such as the top gene CENPA (Amato et al., Mol Cancer 8, 119 (2009)), as well as MAD2L1 (Sotillo et al., Nature 464, 436-440 (2010)) and TPX2 (Heidebrecht et al., Mol Cancer Res 1, 271-279 (2003)), has also been independently previously found associated with CIN. Included in the mitotic CIN attractor are key components of mitotic checkpoint signaling (Orr-Weaver et al., Nature 392, 223-224 (1998)), such as BUB1B, MAD2L1 (aka MAD2), CDC20, and TTK (MSP1). It was recently found (Birkbak et al., Cancer Res 71, 3447-3452 (2011)) that the CIN70 signature is most strongly associated with poor outcome at intermediate, rather than extreme levels. This is consistent with the concept that, while cancer cells are intolerant of extreme instability, moderate mitotic chromosomal instability may provide a proliferative advantage.

Among transcription factors, MYBL2 (aka B-Myb) and FOXM1 were found to be strongly associated with the attractor. They are already known to be sequentially recruited to promote late cell cycle gene expression to prepare for mitosis. (Sadasivam et al., Genes & development 26, 474-489 (2012)).

Inactivation of the retinoblastoma (RB) tumor suppressor promotes CIN (Manning et al., Nat Rev Cancer 12, 220-226 (2012)) and the expression of the attractor signature. Indeed, a similar expression of a “proliferation gene cluster” (Rosty et al., Oncogene 24, 7094-7104 (2005)) was found strongly associated with the human papillomavirus E7 oncogene, which abrogates RB protein function and activates E2F-regulated genes. Consistently, many among the genes of the attractor correspond to E2F pathway genes controlling cell division or proliferation. Among the E2F transcription factors, E2F8 and E2F7 were found to be most strongly associated with the attractor.

### 4.2.3. A Lymphocyte-Specific Attractor Metagene

A strong lymphocyte-specific attractor was identified as consisting mainly of genes CD53, PTPRC, LAPTM5, DOCK2, EVI2B, CYBB and LCP2. This attractor is strongly associated with the expression of miR-142 as well as with particular hypermethylated and hypomethylated gene signatures. (Andreopoulos, B. & Anastassiou, D., Cancer Informatics 11, 61-75 (2012)). The latter include many of the overexpressed genes, suggesting that their expression is triggered by hypomethylation. Gene set enrichment analysis reveals that the attractor is found enriched in genes known to be preferentially expressed in lymphocyte differentiation and is also found occasionally upregulated in various cancers. (Lee et al., International Immunology 16, 1109-1124 (2004)).

### 4.2.4. An Endothelial Attractor Metagene

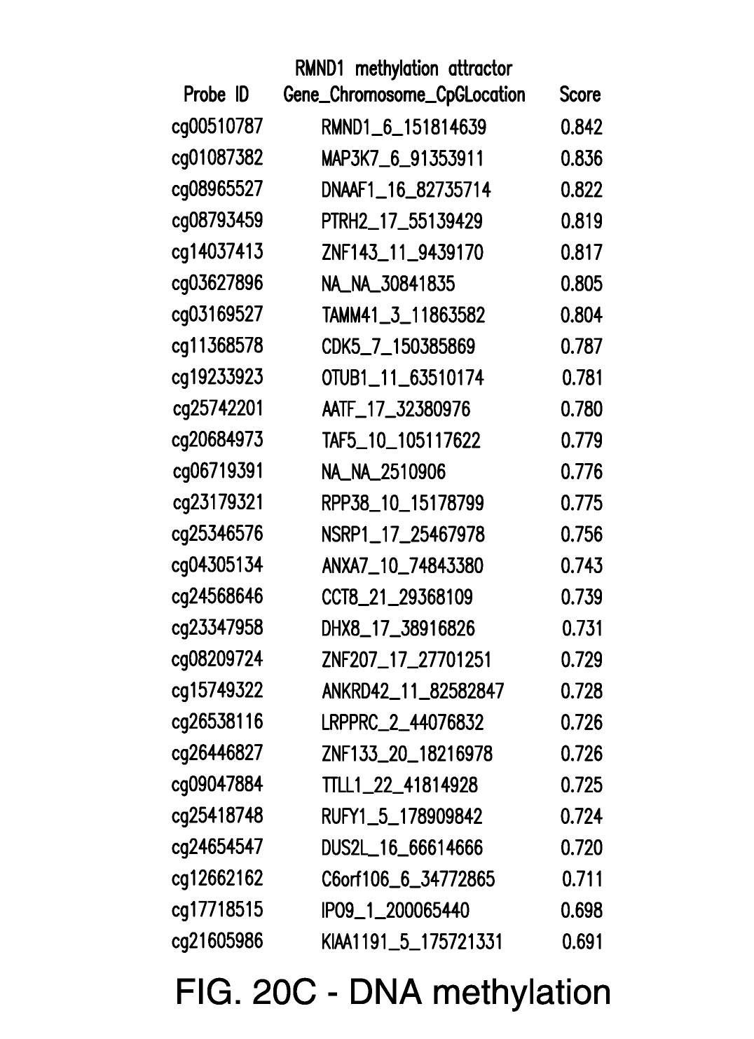

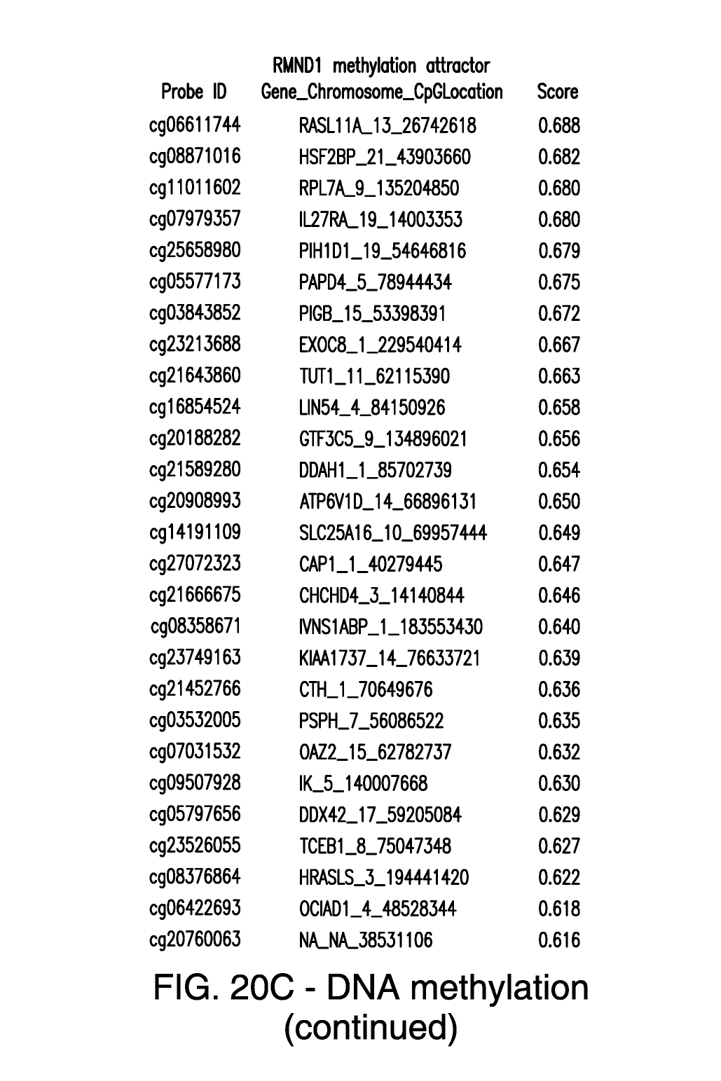

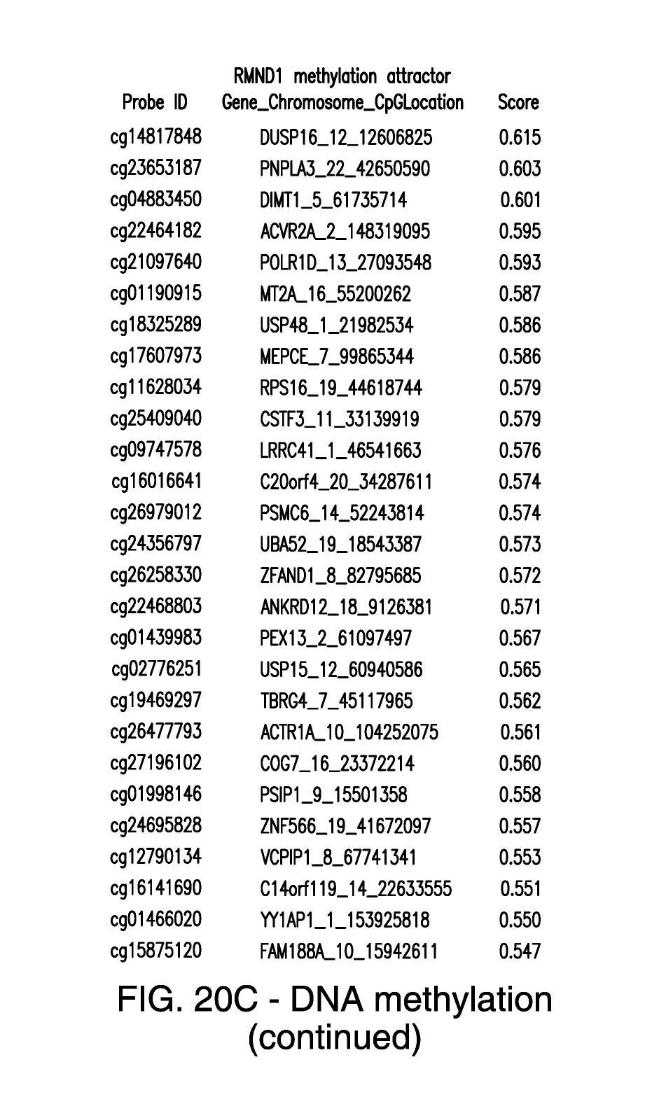

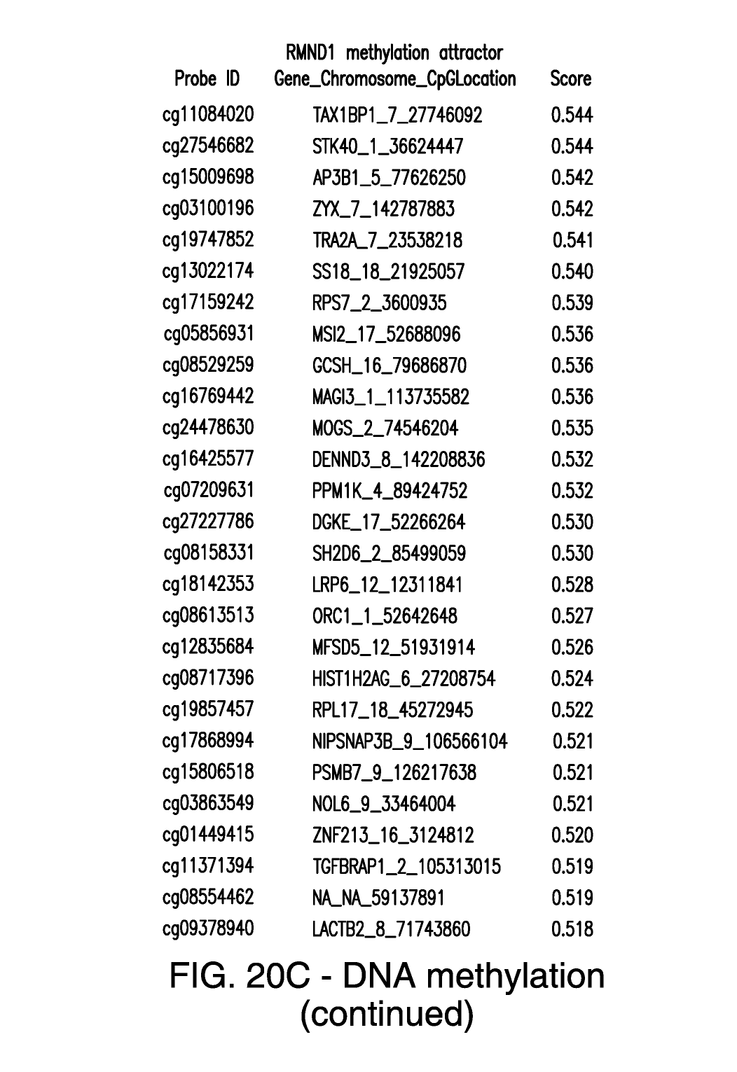

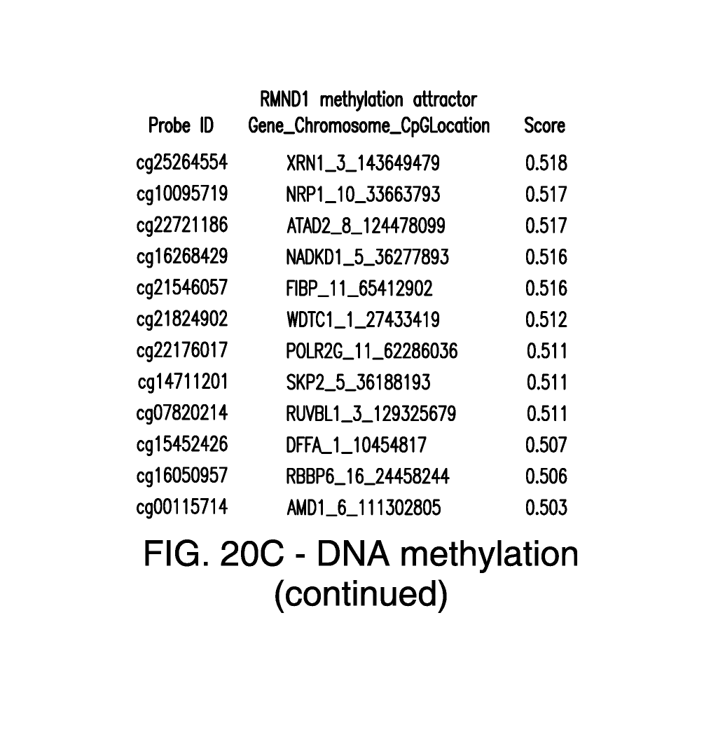

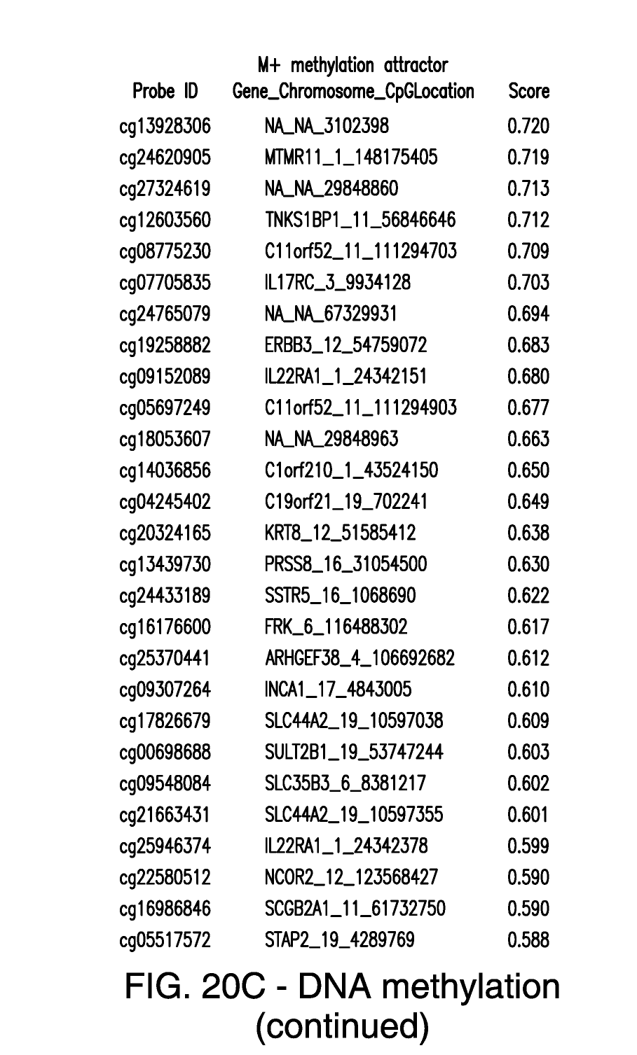

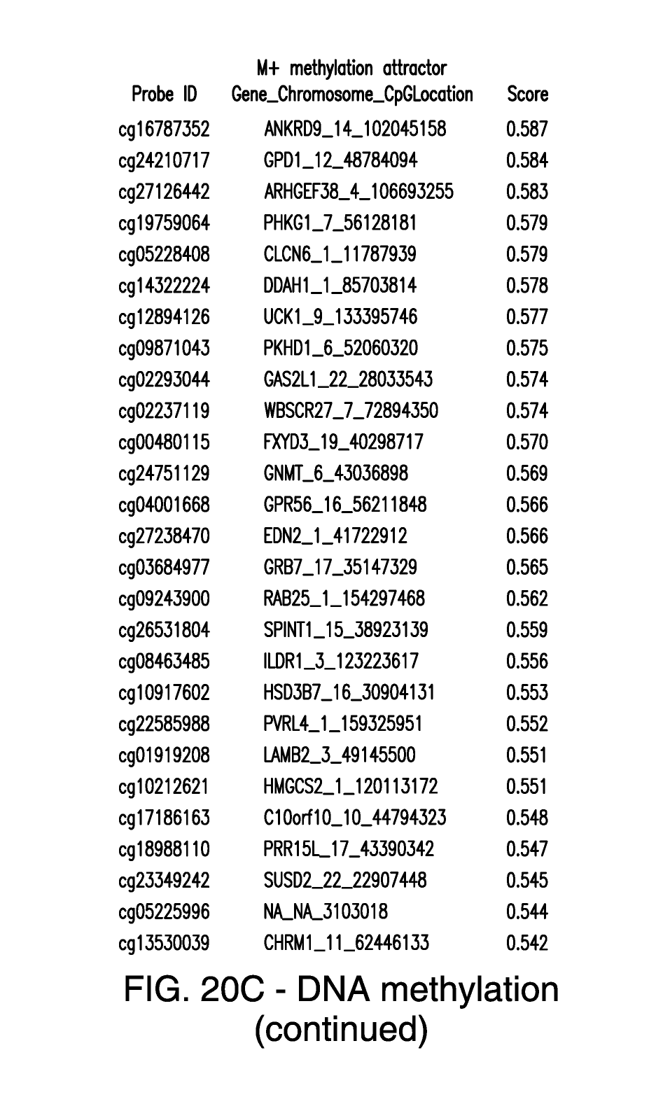

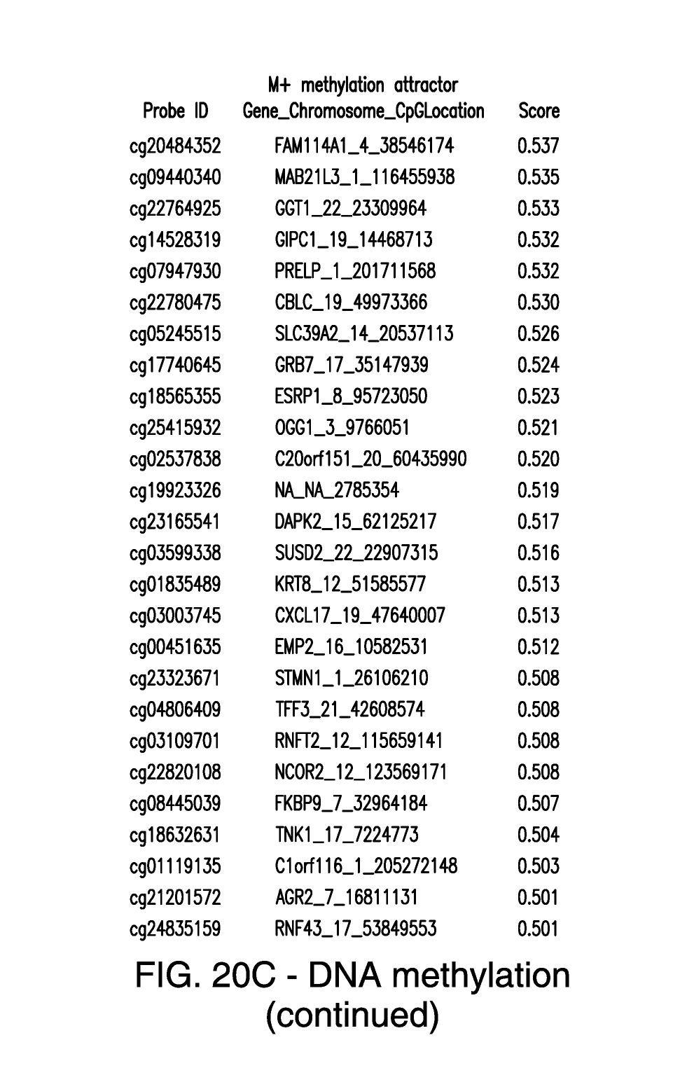

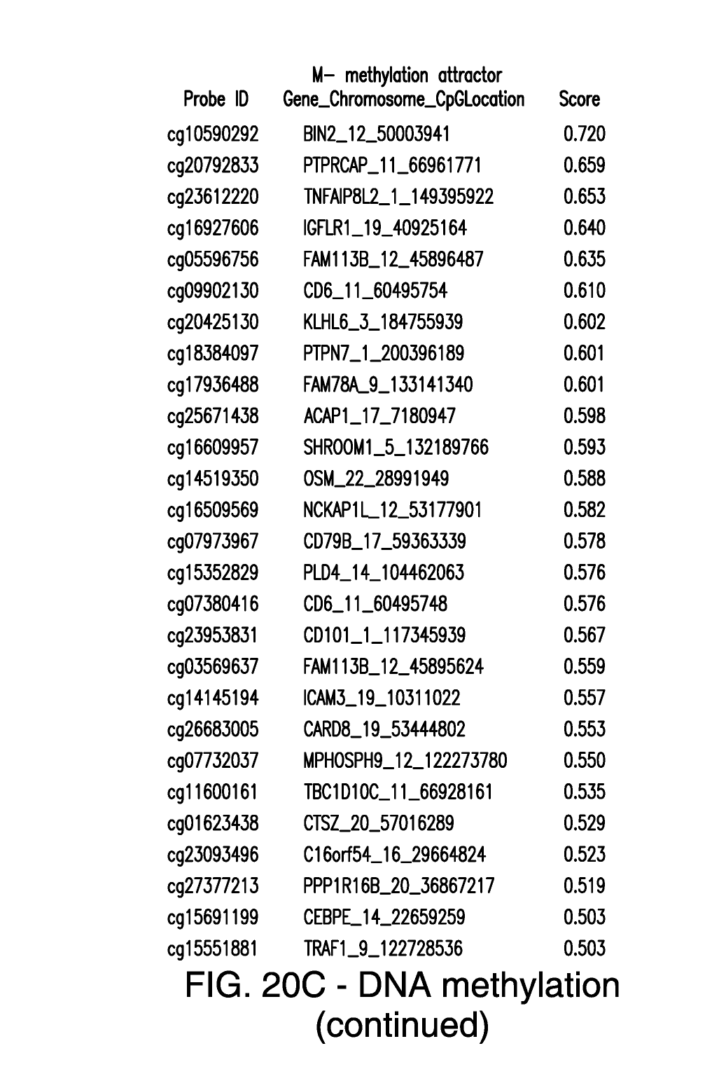

A novel attractor metagene contains endothelial markers and is associated with angiogenesis (END). The top-ranked genes of the END attractor metagene are CDH5, ROBO4, CXorf36, CD34, CLEC14A, ARHGEF, CD93, CLEC14A, ARHGEF15, CD93, LDB2, ELTD, MYCT1. Nearly all these genes are endothelial markers. The top gene, CDH5, codes for VE-cadherin, which is known to be involved in a pathway suppressing angiogenic sprouting (Abraham, S. et al. Curr Biol 19, 668-74 (2009)). The second gene, ROBO4, is known to inhibit VEGF-induced pathologic angiogenesis and endothelial hyperpermeability (Jones, C. A. et al. Nat Med 14, 448-53 (2008)). Consistently, the END attractor metagene appears to be protective and anti-angiogenic, stabilizing the vascular network. For example, 22 out of the 27 genes of the END attractor are among the 265 genes included in FIGS. 20A-D as most associated with patients' survival in a recent study (Wozniak, M. B. et al. PLoS One 8, e57886 (2013)) of renal cell carcinoma (P<8.4×10⁻³⁸ based on Fisher's exact test). These good-prognosis genes were intermixed in the same file with many poor-prognosis genes of the CIN attractor, suggesting that the CIN and END attractor metagenes are two of the most prognostic features in renal cell carcinoma.

Interestingly, the MES and END attractor metagenes are positively associated with each other (FIGS. 20A-D), in the sense that overexpression of the END signature tends to imply overexpression of the MES signature and vice-versa. This is consistent with mutual exclusivity between angiogenesis and invasiveness and with related findings (Lu, K. V. et al. Cancer Cell 22, 21-35 (2012)) that VEGF inhibits tumor cell invasion and mesenchymal transition, while antiangiogenic therapy is associated with increased invasiveness (Paez-Ribes, M. et al. Cancer Cell 15, 220-31 (2009)). It may also explain the paradoxical protective nature of signatures related to the MES attractor metagene in invasive breast cancers (Beck, A. H., Espinosa, I., Gilks, C. B., van de Rijn, M. & West, R. B. Lab Invest 88, 591-601 (2008)), as the observed association of proteins such as SPARC with improved clinical outcome may be due the concomitant presence of the END signature. Indeed, it was found that SPARC, a key member of the MES signature, is also among the top 100 genes most associated with the END signature.

### 4.2.5. Methylation Attractor Molecular Signatures

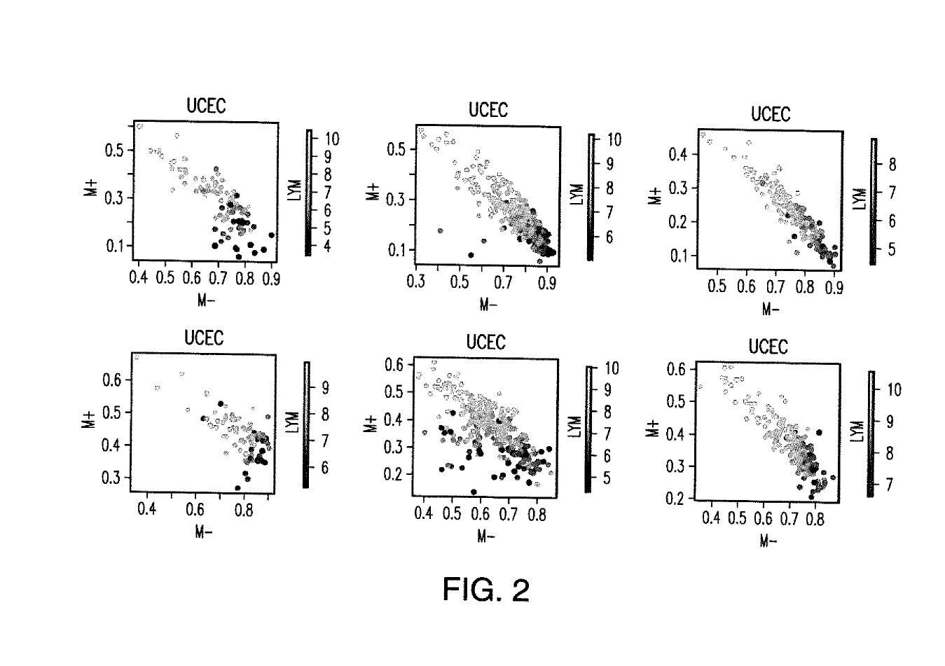

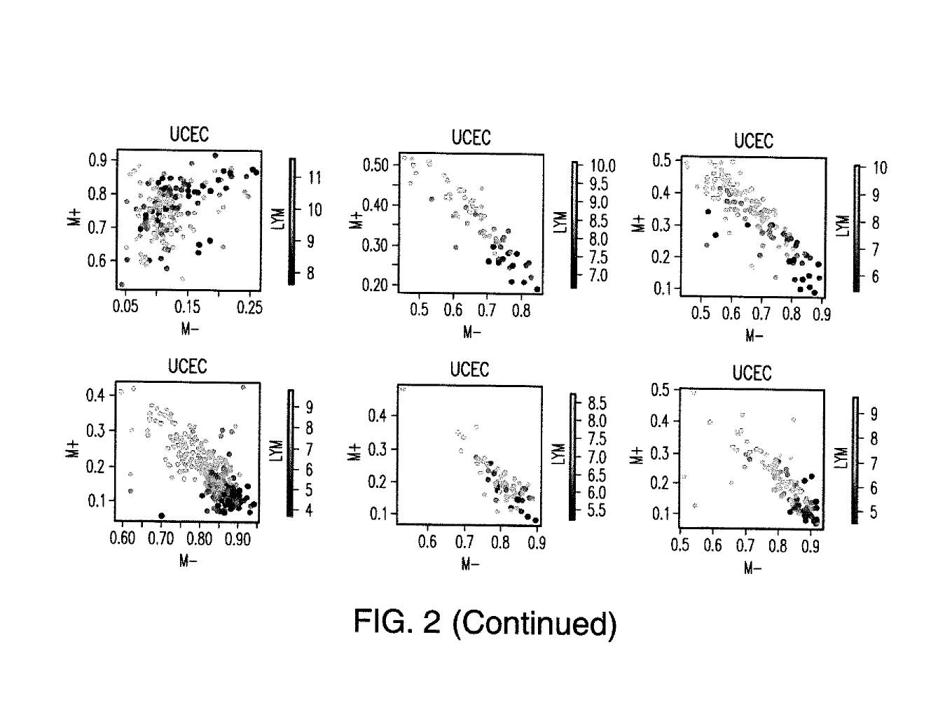

Two methylation attractor molecular signatures were observed that had a strong reverse association with each other, in the sense that the absence of one implied the strong presence of the other, or they were both present at intermediate levels. It was also found that they are strongly associated with the lymphocyte-specific LYM attractor metagene. These two methylation molecular signatures are referred to as M+ and M−, the former corresponding to hypermethylated sites in the presence of the LYM signature, and the latter corresponding to a hypomethylated site in the presence of the LYM signature. Six among the 27 genes of the M− signature (BIN2, TNFAIP8L2, ACAP1, NCKAP1L, FAM78A, PTPN7) are also among the 168 genes listed in the LYM attractor metagene (P<9.21×10⁻⁷ based on Fisher's exact test), suggesting that the LYM signature is at least partly triggered by the hypomethylation of the M− signature. FIG. 2 demonstrates, in the form of 12 scatter plots, this remarkable “methylation switch” and the association between LYM, M+ and M− signatures in all cancer types except leukemia. These results are consistent with previous findings (Andreopoulos, B. & Anastassiou, D. Cancer Inform 11, 61-75 (2012)) associating these signatures with the microRNA miR-142, but the instant results indicate that this association of the LYM signature with M+ and M− appears to be strongly present in all solid cancer types. Given that the LYM signature is strongly protective in ER-negative breast cancers (Cheng, W. Y., Ou Yang, T. H. & Anastassiou, D. Sci Trans1 Med 5, 181ra50 (2013)), further investigating the mechanisms behind these methylation signatures is a particularly promising area for further research.

### 4.2.5. Additional Attractor Molecular Signatures

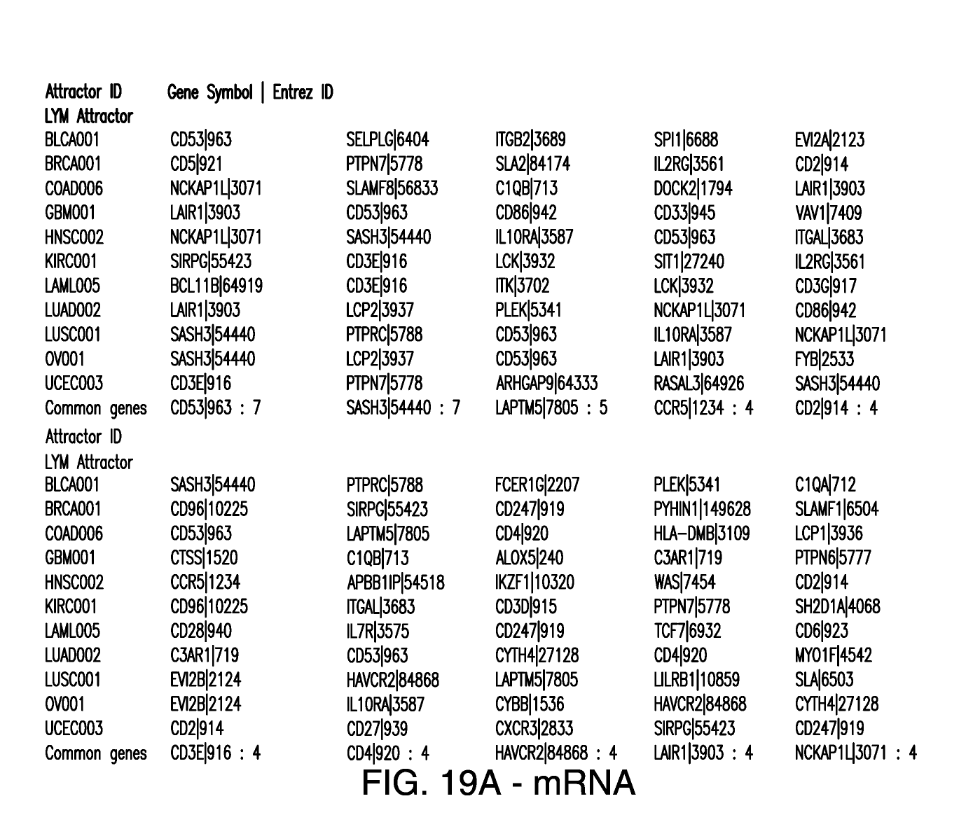

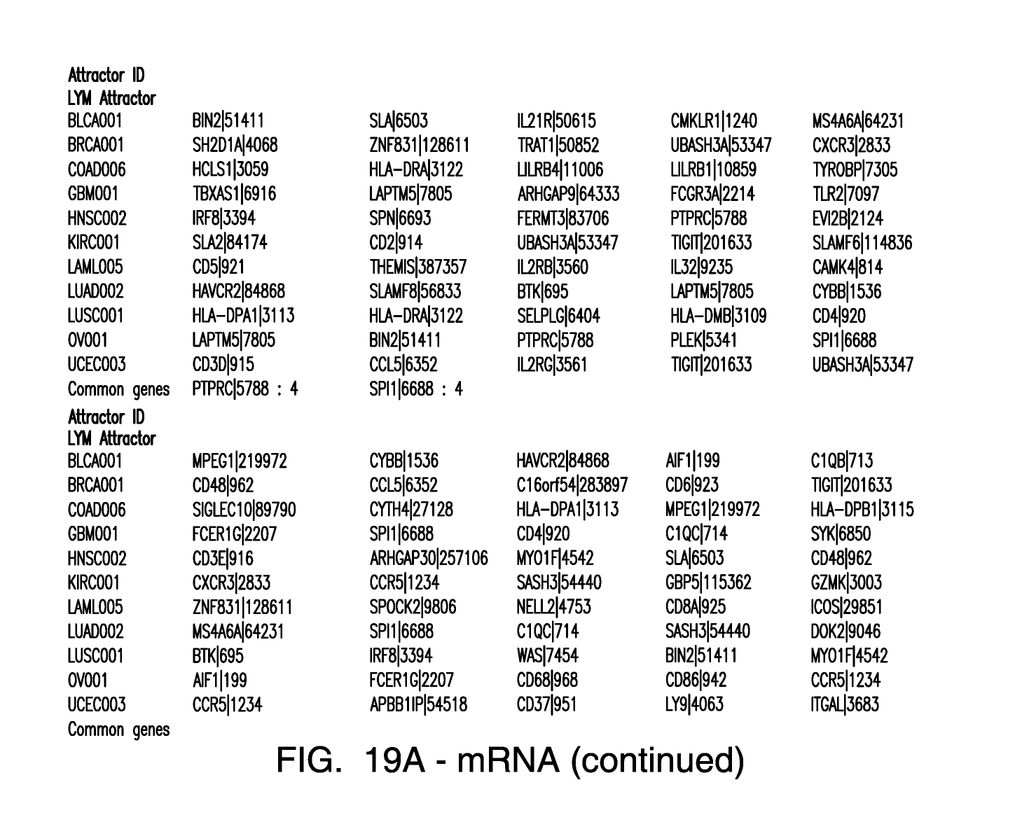

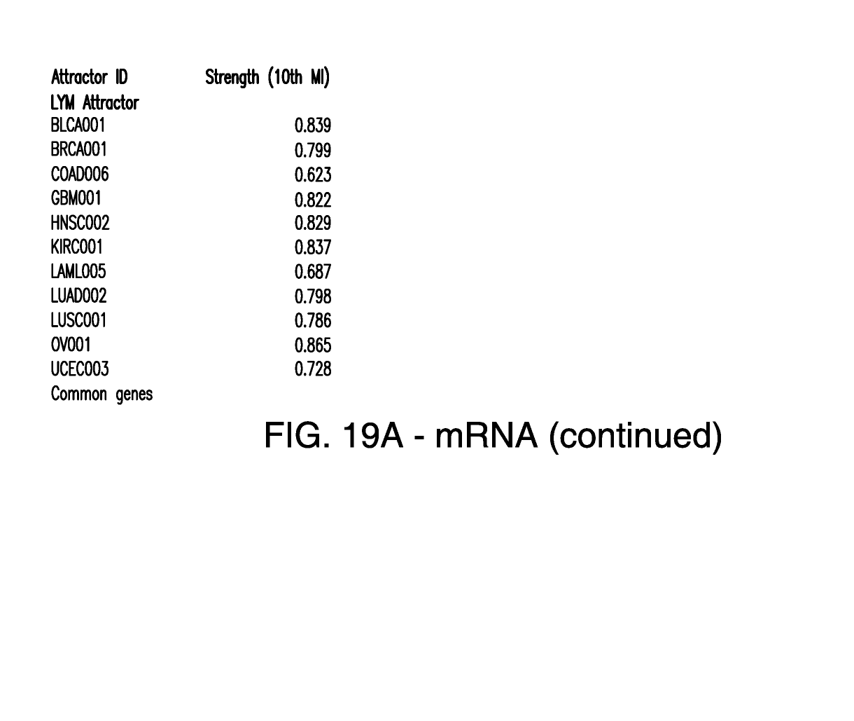

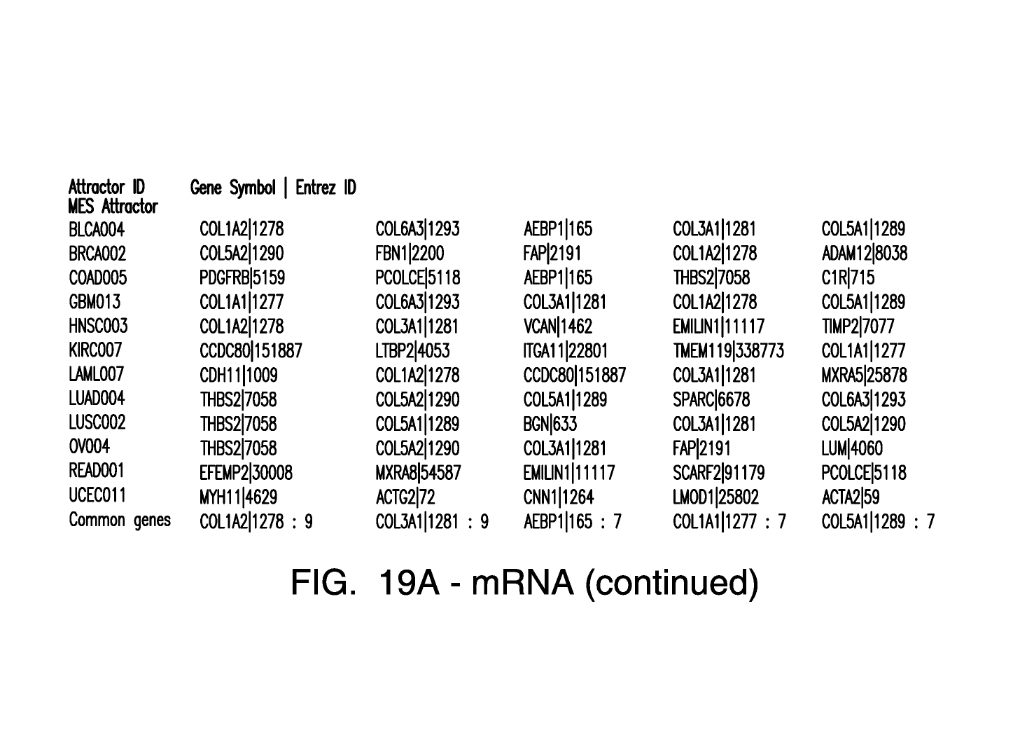

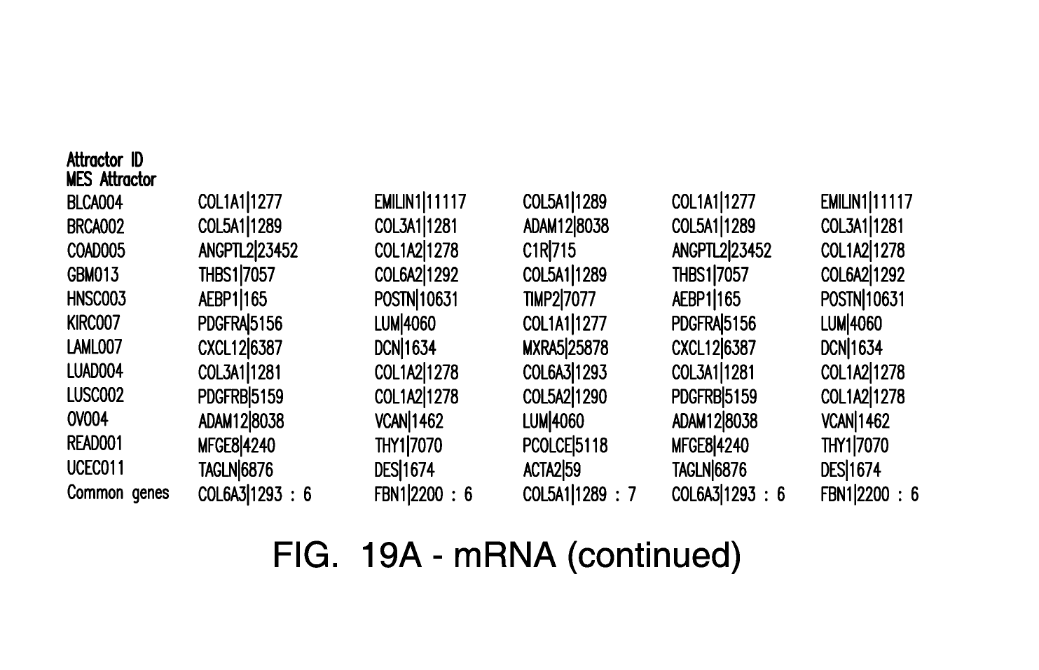

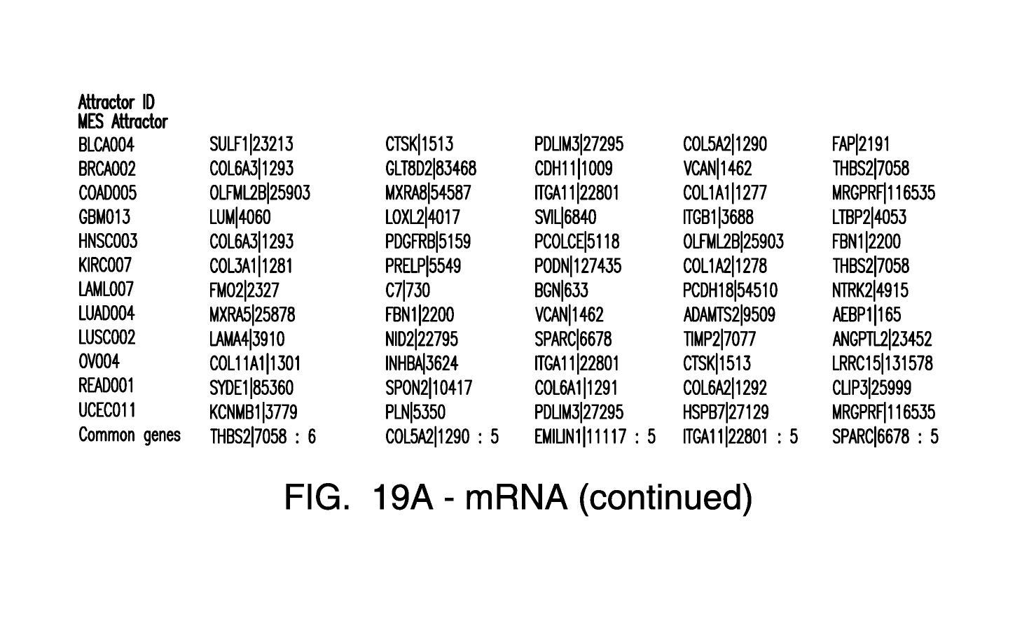

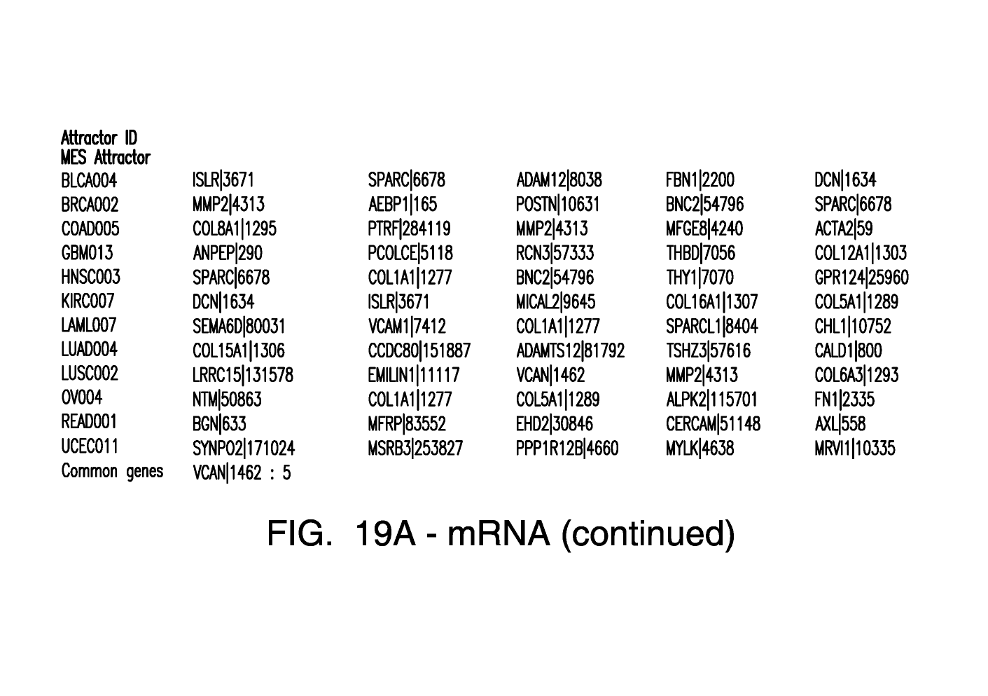

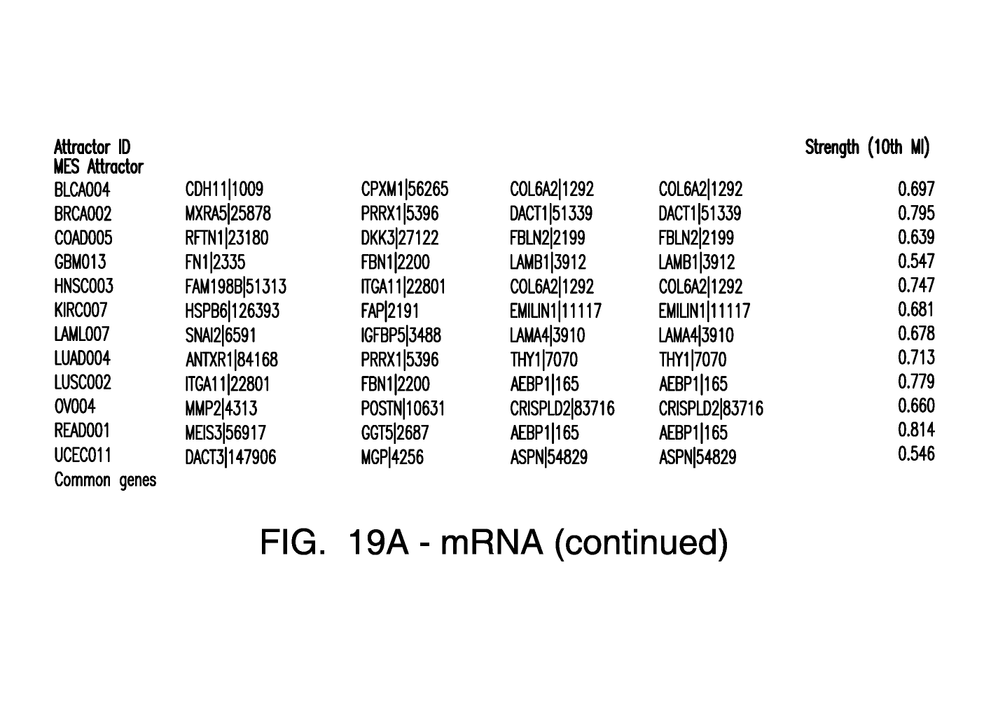

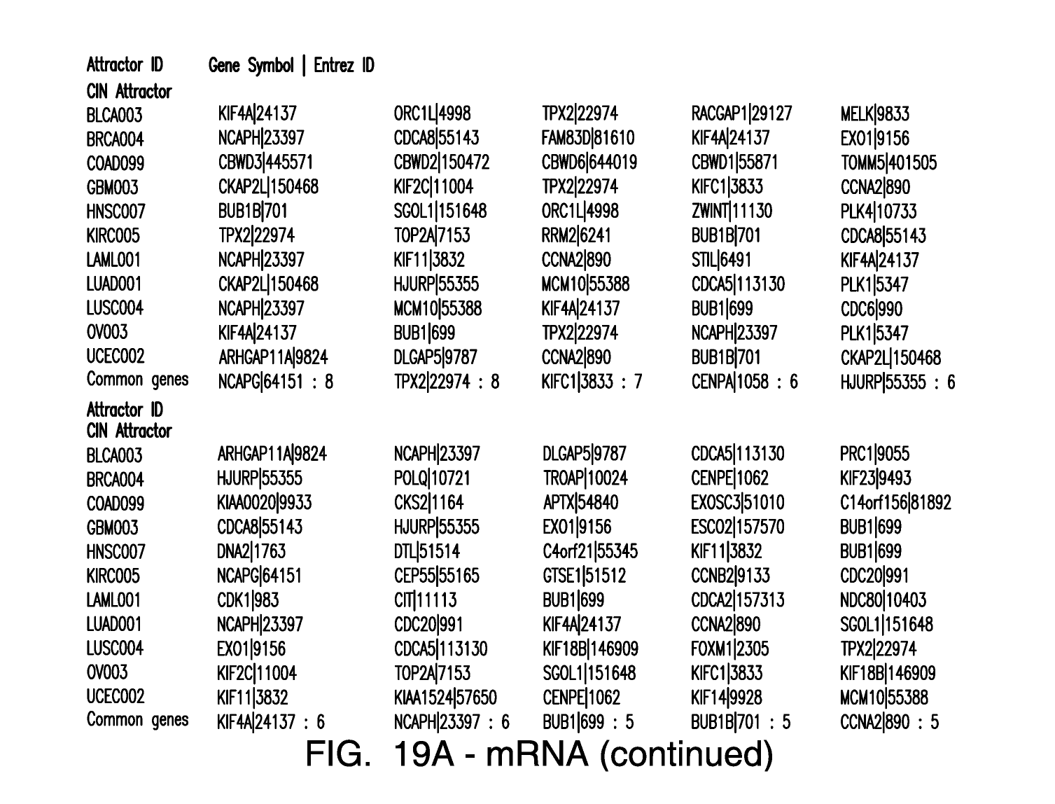

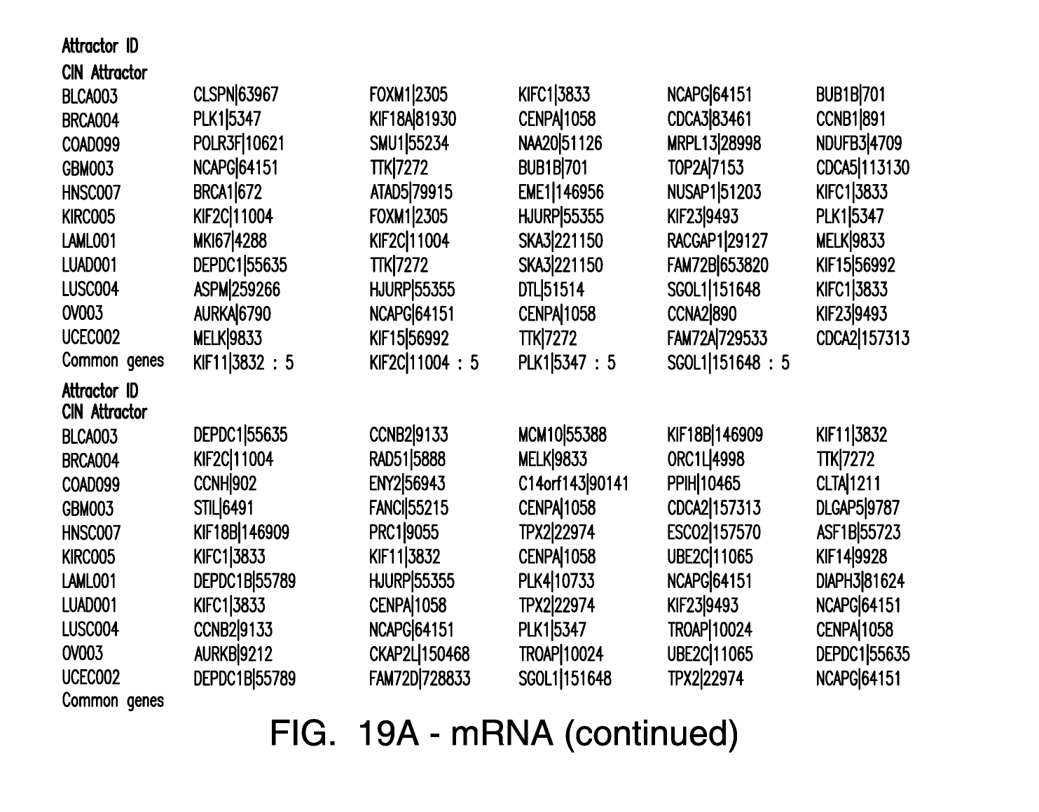

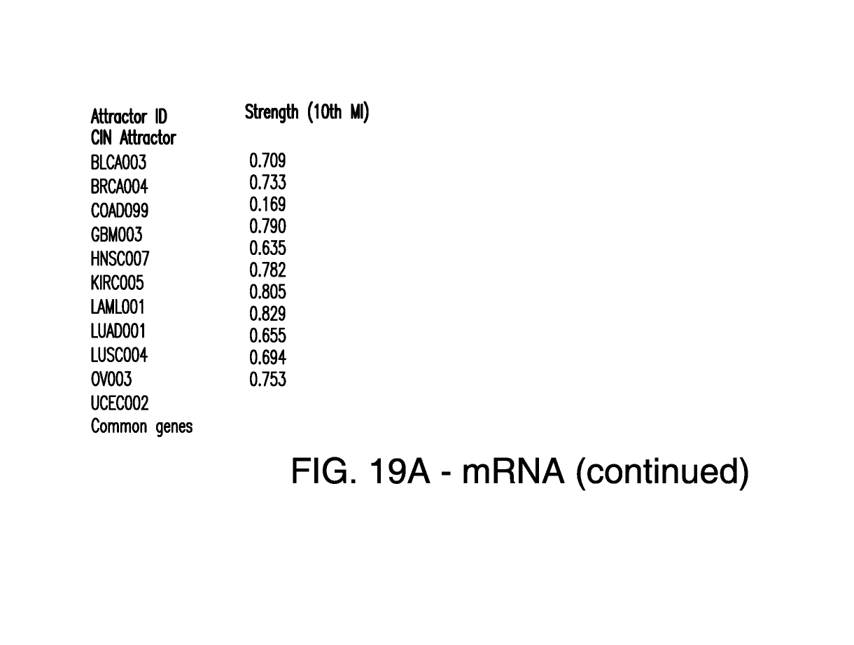

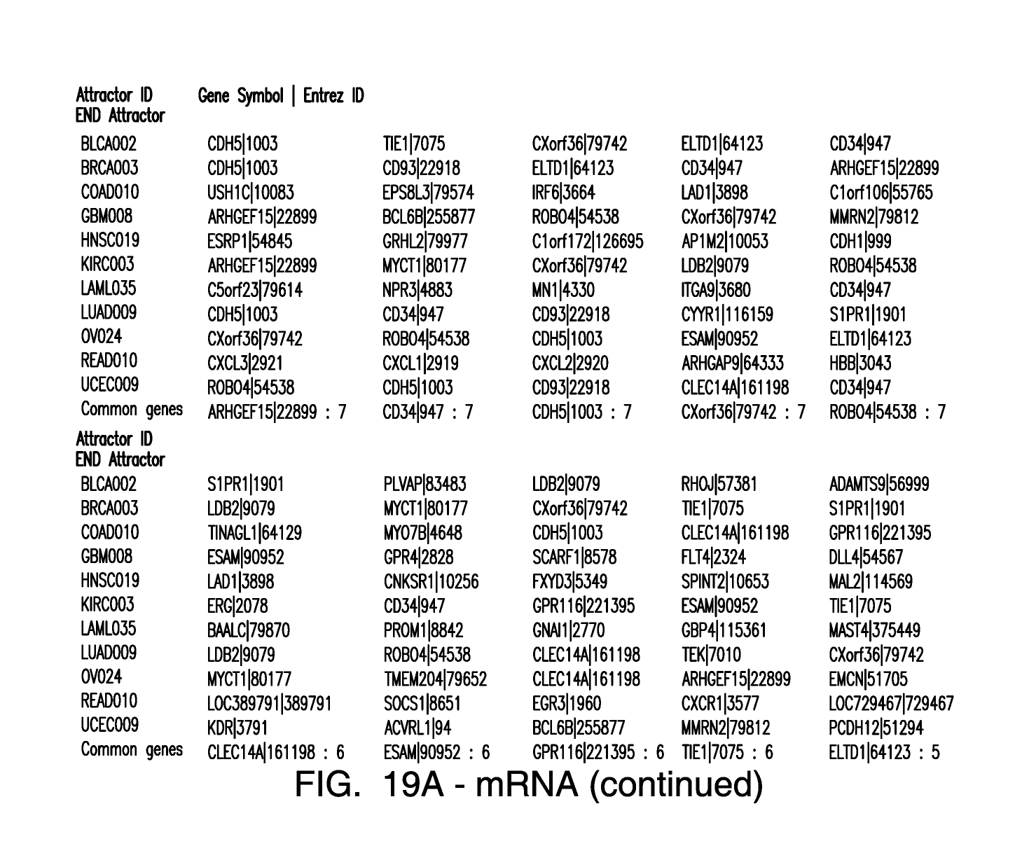

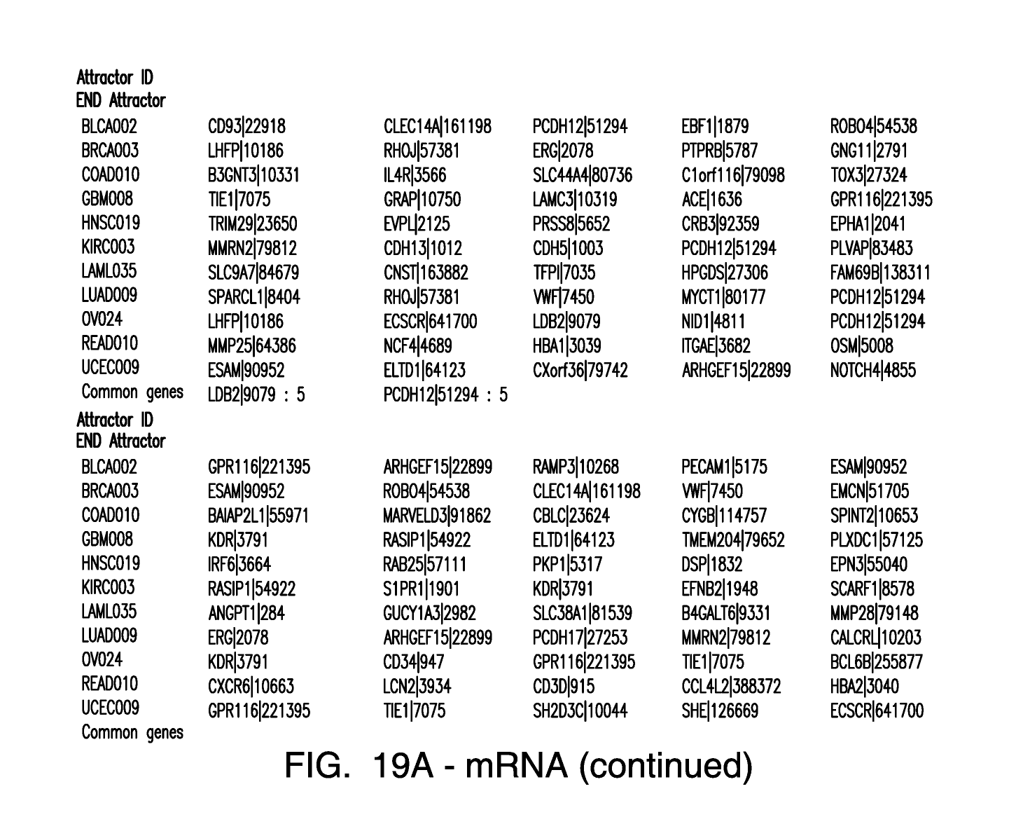

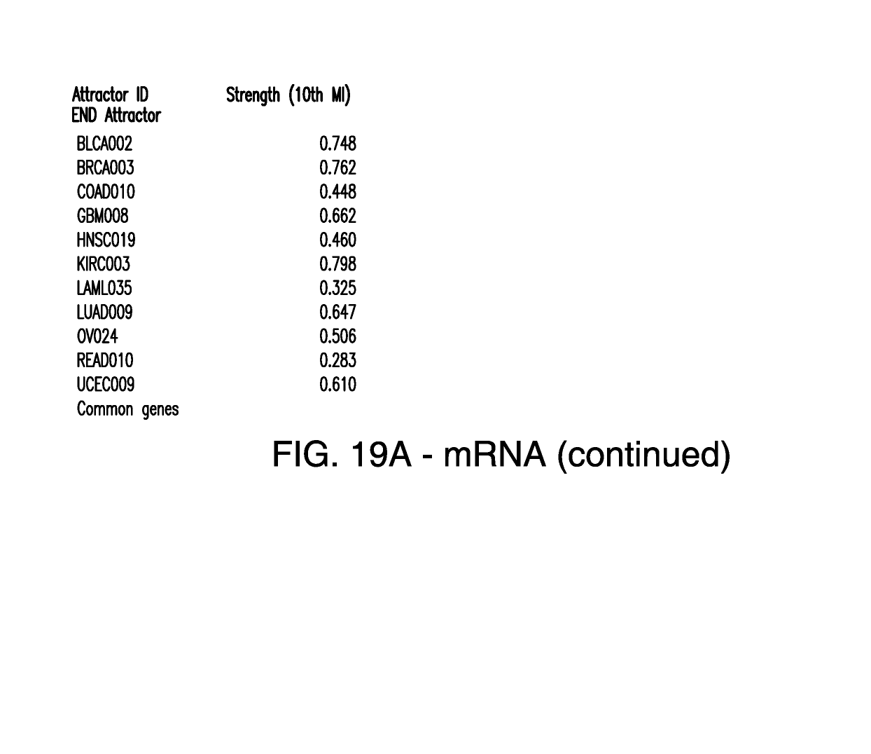

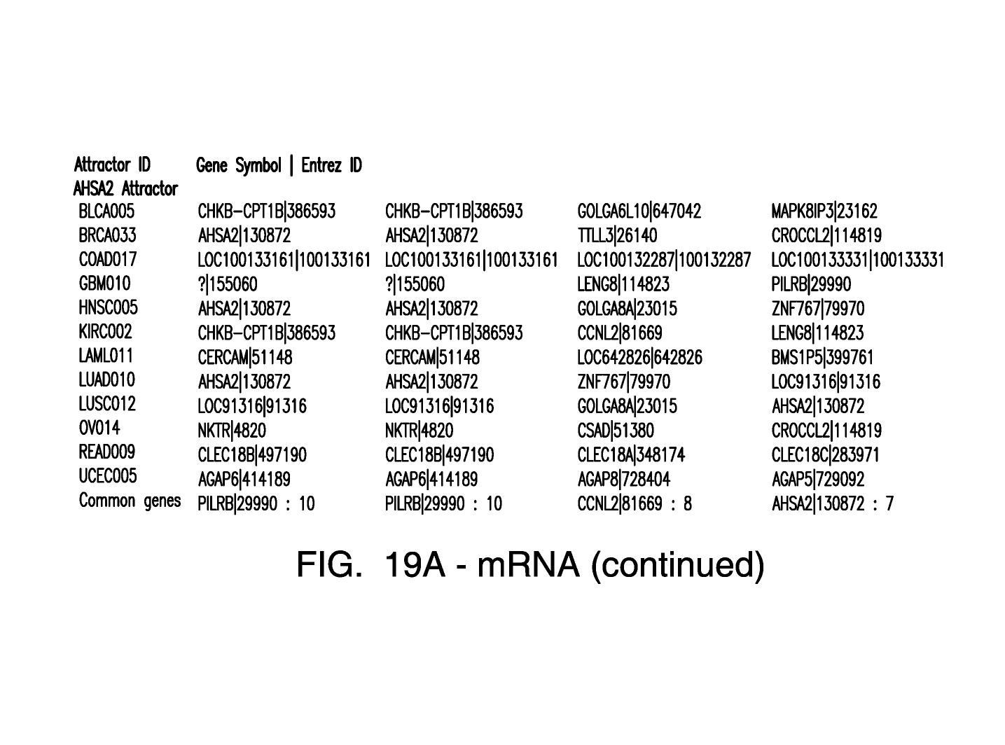

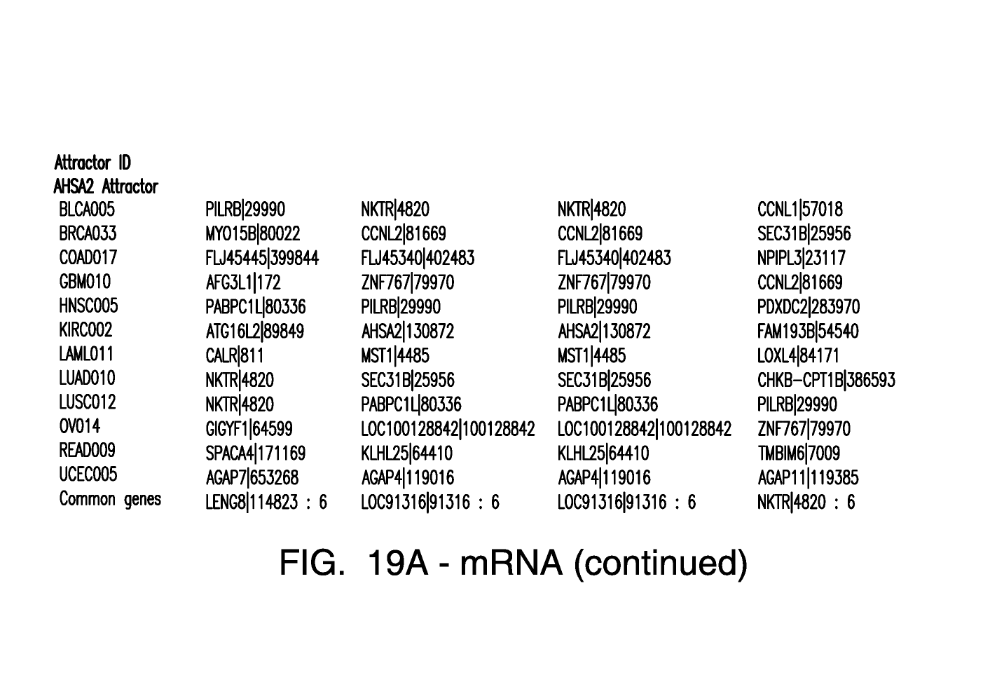

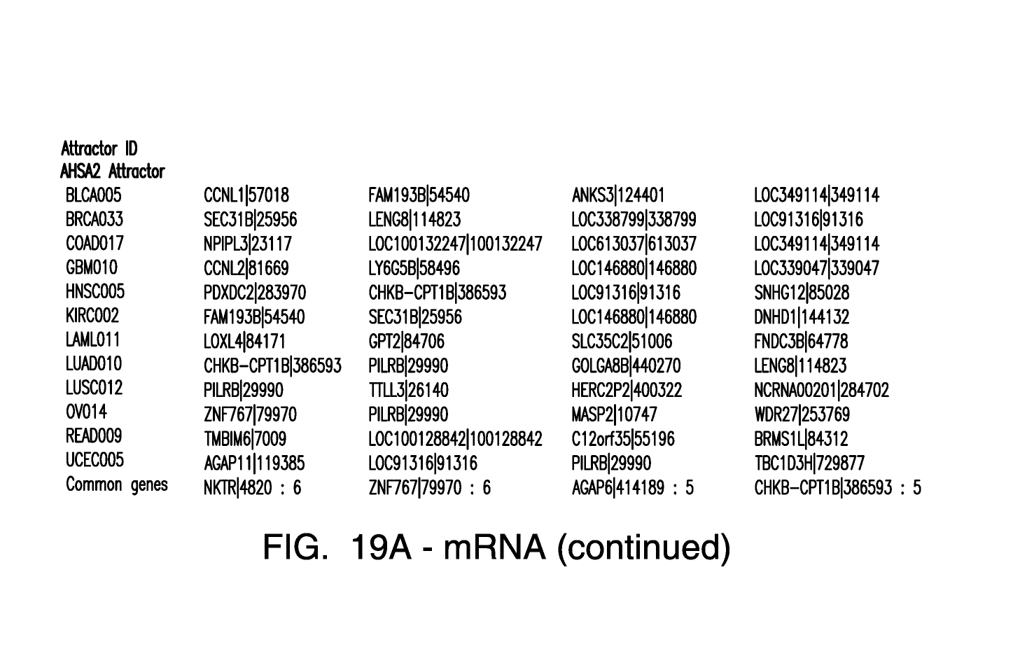

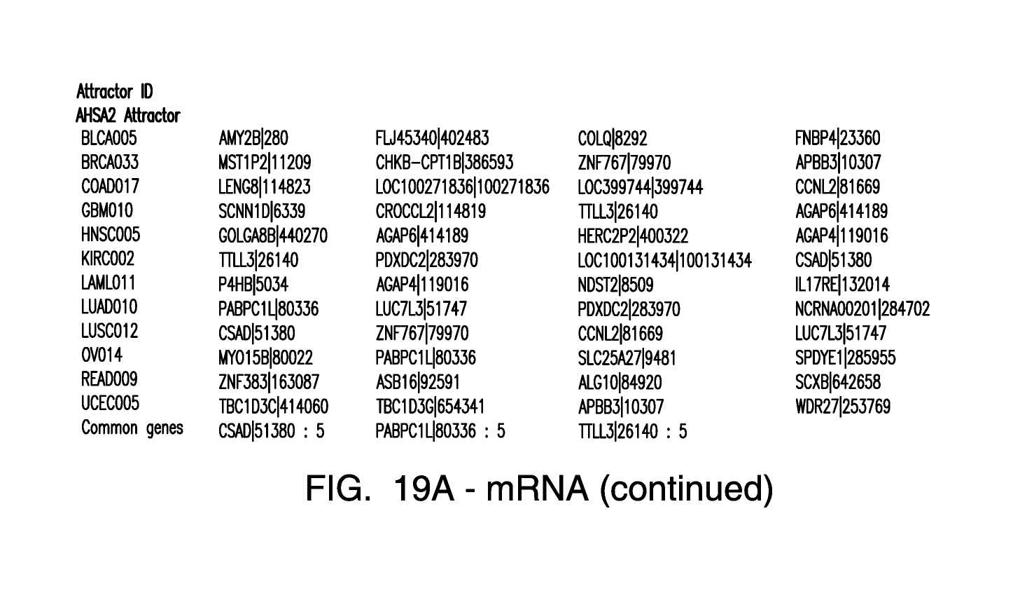

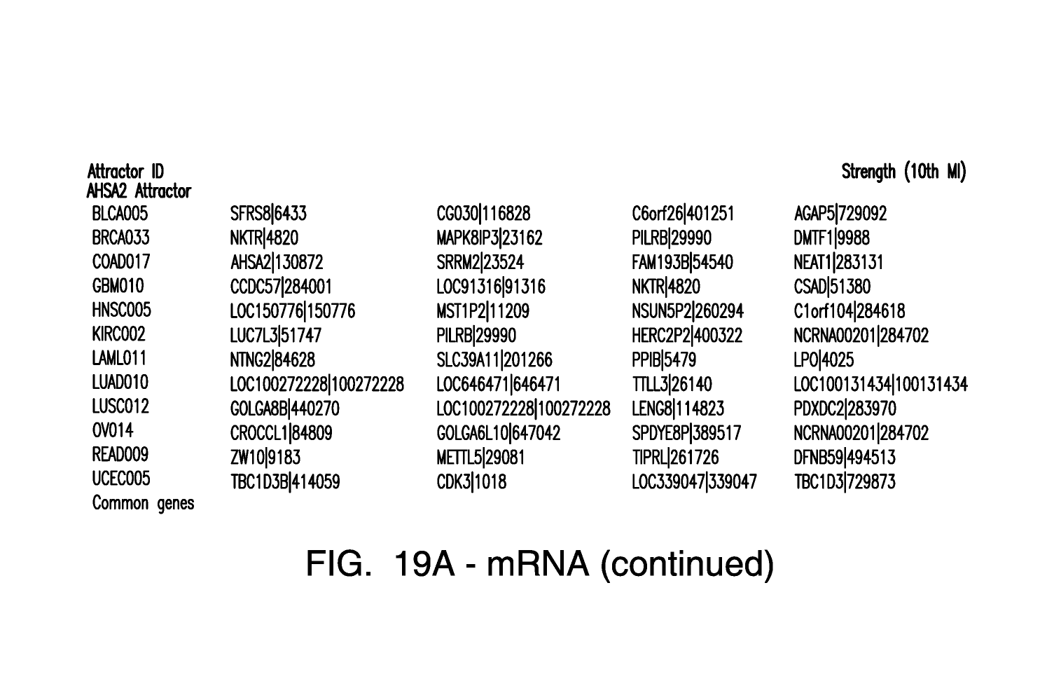

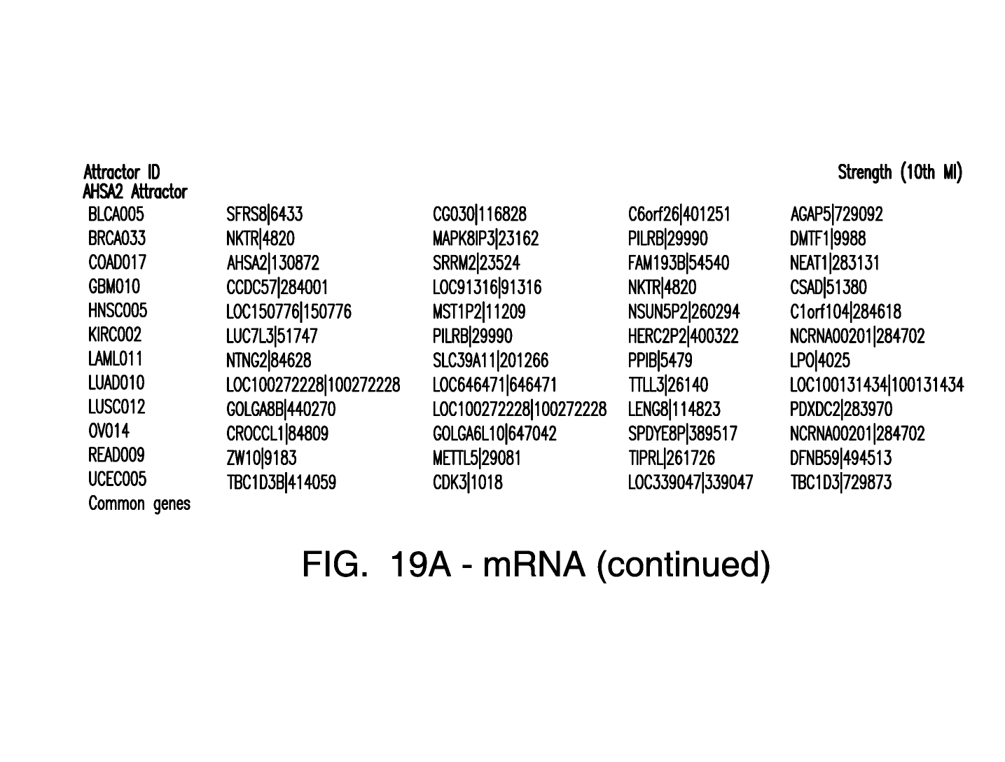

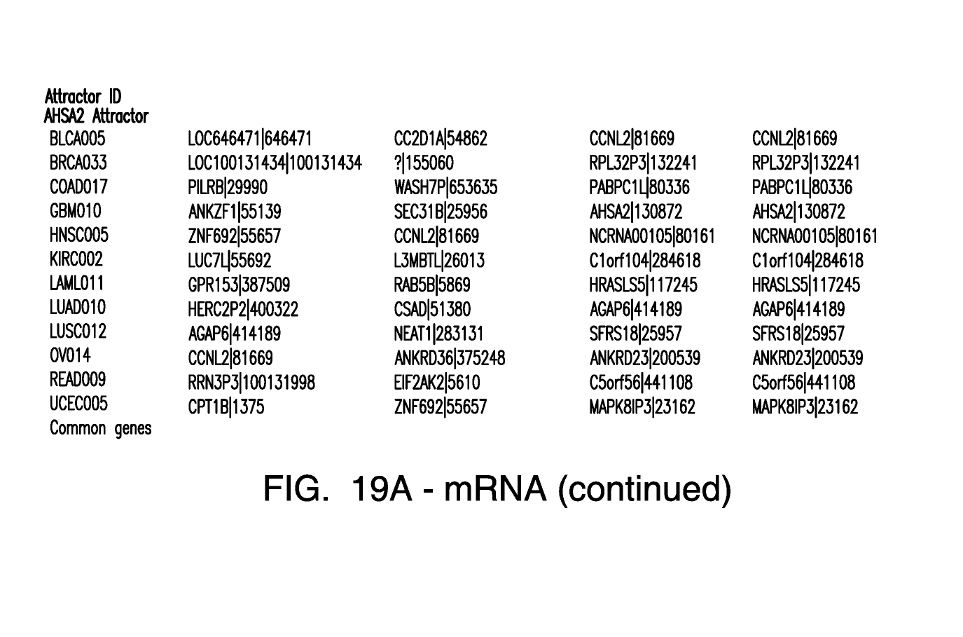

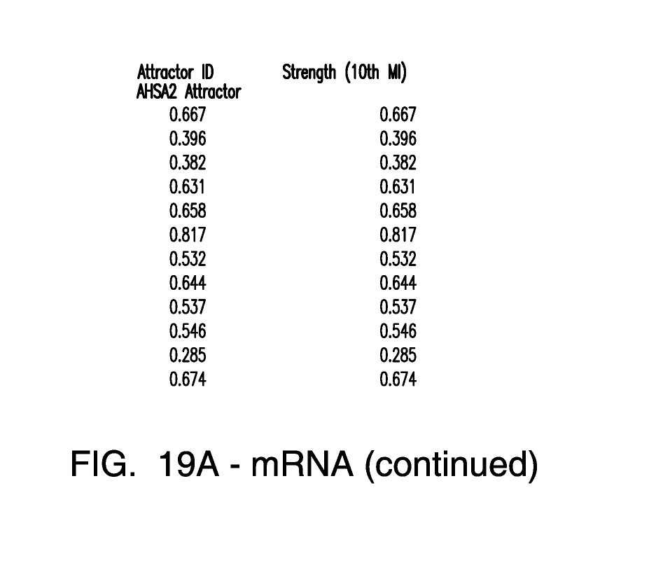

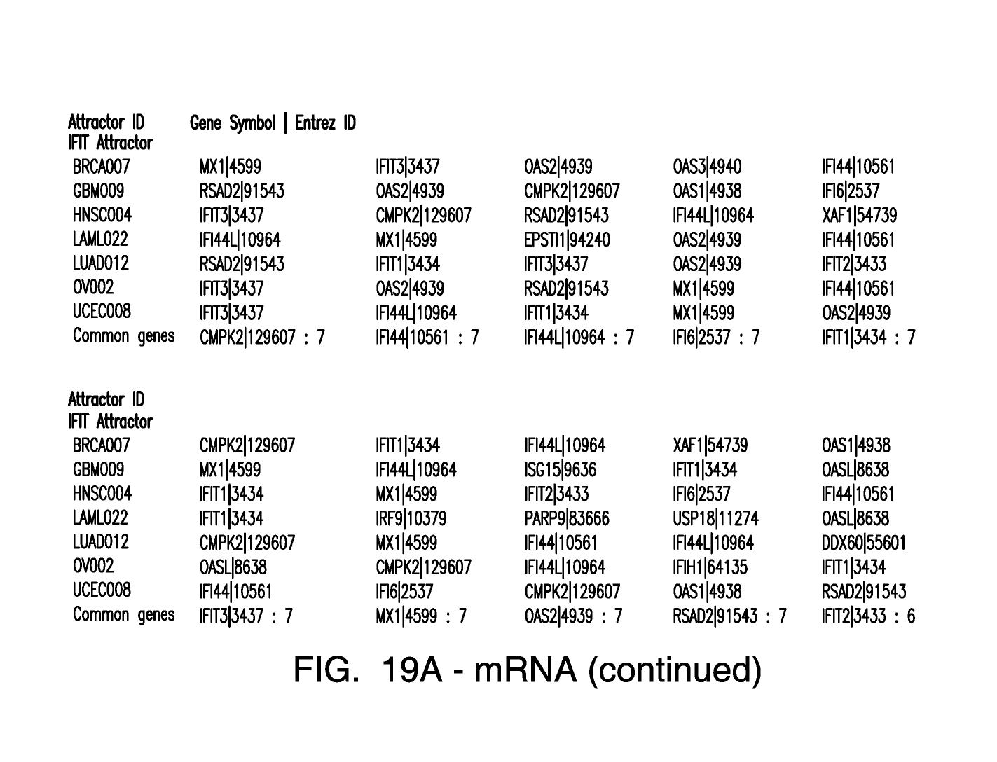

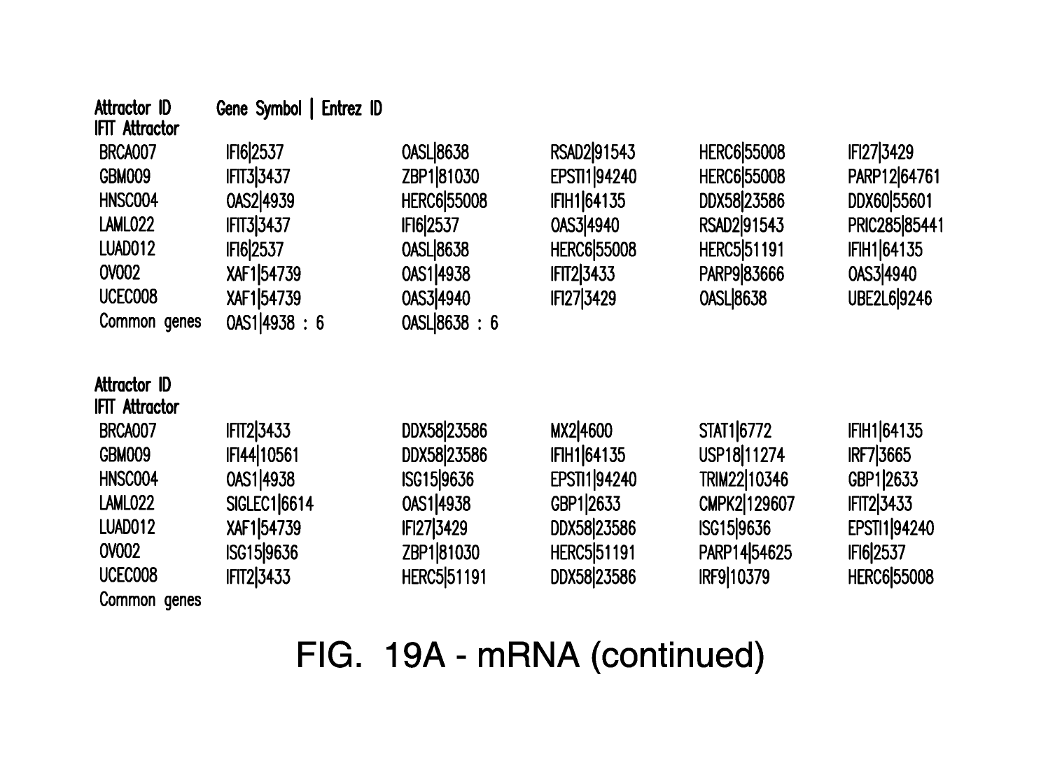

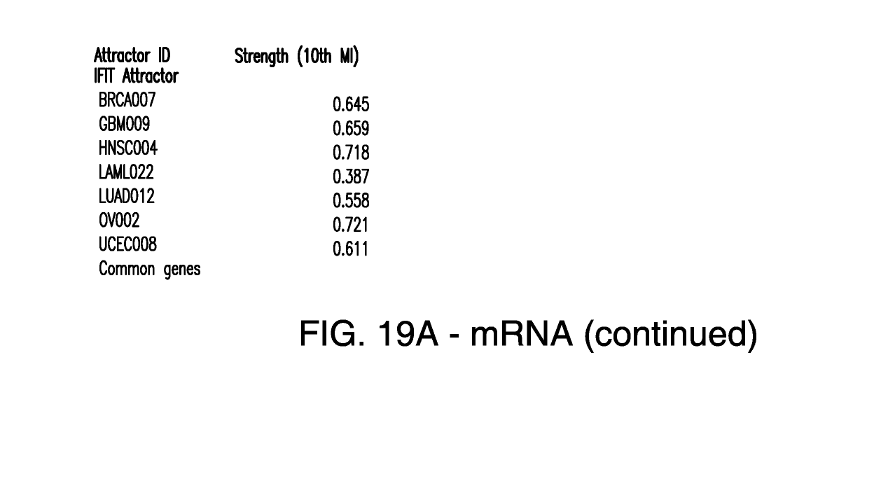

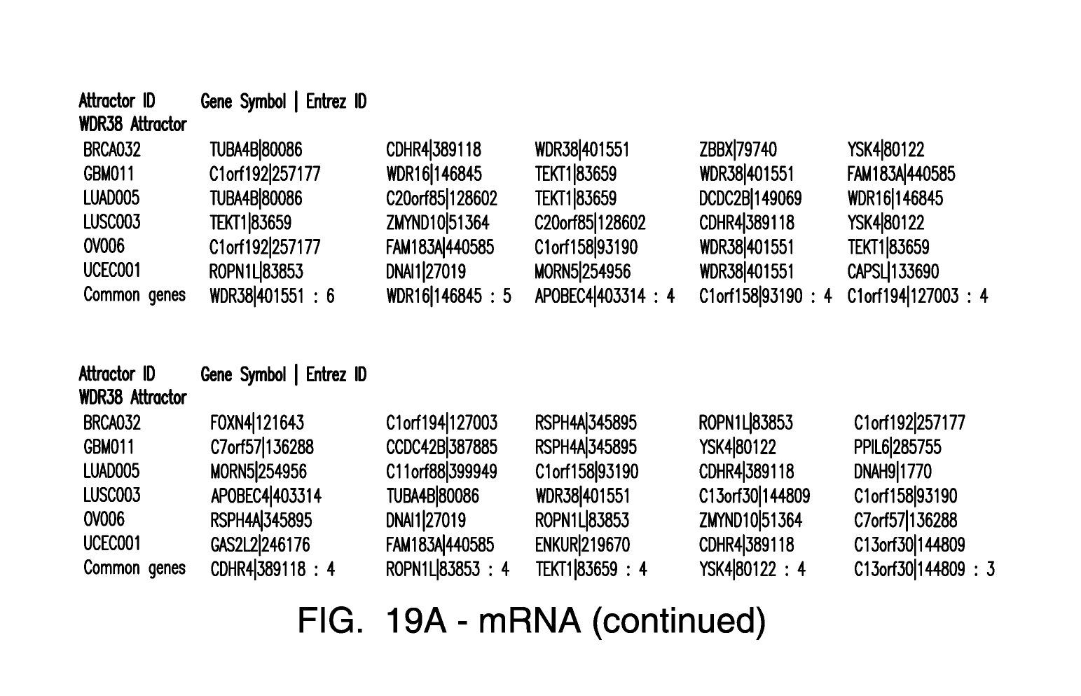

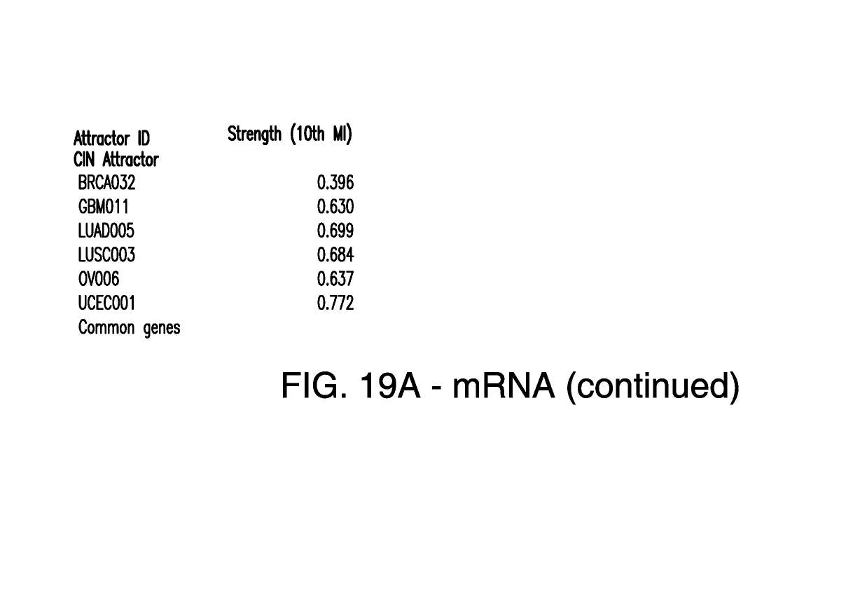

Including the attractor molecular signatures described above, at total of 15 attractor molecular signatures were identified using the TCGA pancan12 data sets. Seven of which were present in protein-coding gene expression data sets, three in methylation data sets, three in microRNA expression data sets, and two in protein activity data sets. Complete information concerning the identity of the individual genes making up the 15 attractor molecular signatures is presented in FIGS. 19A-D, 20A-D, and 21A-D.

### 4.2.6. BCAM Assay

An assay incorporating attractor molecular signatures described herein is identified herein as BCAM (Breast Cancer Attractor Metagenes). BCAM has the unexpected and remarkable characteristic that (a) it does not make any use of ER, PR and HER2 status or molecular subtype classification, none of which provided additional prognostic value in the experiments describe herein (Example 2), and (b) it is universally applicable to all subtypes and stages of breast cancer. BCAM is composed of several molecular features: the breast cancer specific FGD3-SUSD3 metagene, four attractor metagenes present in multiple cancer types (CIN, MES, LYM, and END associated with mitotic chromosomal instability, mesenchymal transition, lymphocyte infiltration, and endothelial markers, respectively), three additional individual genes (CD68, DNAJB9 and CXCL12), tumor size, and the number of positive lymph nodes. Based on analysis using several independent data sets, BCAM's prognostic predictions can outperform those resulting from existing commercial breast cancer biomarker assays.

### 4.3. Diagnosis & Treatment with Attractor Molecular Signatures

### 4.3.1. Methods of Diagnosis & Treatment Generally

Conventional gene expression analysis in connection with cancer diagnosis and treatment has resulted in several cancer types being further classified into subtypes labeled, e.g. as “mesenchymal” or “proliferative.” Such characterizations, however, may sometimes simply reflect the presence of the mesenchymal transition attractor or the mitotic chromosomal instability attractor, respectively, in some of the analyzed samples. Similar subtype characterizations across cancer types often share several common genes, but the consistency of these similarities has not been significantly high.

In contrast, by using an unconstrained algorithm independent of subtype classification or dimensionality reduction, as described herein, several attractors exhibiting remarkable consistency across many cancer types can be identified, indicating that each of them represents a precise biological phenomenon present in multiple cancers and therefore are of particular use in cancer diagnosis and treatment.

For example, the mesenchymal transition attractor described above is significantly present only in samples whose stage designation has exceeded a threshold, but not in all of such samples. Similarly, the mitotic chromosomal instability attractor described above is significantly present only in samples whose grade designation has exceeded a threshold, but not in all of them. On the other hand, the absence of the mesenchymal transition attractor in a profiled high-stage sample (or the absence of the mitotic chromosomal instability attractor in a profiled high-grade sample) does not necessarily mean that the attractor is not present in other locations of the same tumor. Indeed, it is increasingly appreciated that tumors are highly heterogeneous. (Gerlinger et al., The New England Journal of Medicine 366, 883-892 (2012)). Therefore it is possible for the same tumor to contain components, in which, e.g., some are migratory having undergone mesenchymal transition, some other ones are highly proliferative, etc. If so, attempts for subtype classification based on one particular site in a sample may be confusing.

Similarly, existing molecular marker products make use of multigene assays that have been derived from phenotypic associations in particular cancer types. For breast cancer, biomarkers such as Oncotype DX (Paik et al., The New England Journal of Medicine 351, 2817-2826 (2004)) and Mammaprint (van't Veer et al., Nature 415, 530-536 (2002)) contain several genes highly ranked in the attractors. For example, many among the genes used for the Oncotype DX breast cancer recurrence score directly converge to one of the identified attractors: MMP11 to the mesenchymal transition attractor; MKI67 (aka Ki-67), AURKA (aka STK15), BIRC5 (aka Survivin), CCNB1, and MYBL2 to the mitotic chromosomal instability attractor; CD68 to the lymphocyte-specific attractor; ERBB2 and GRB7 to the HER2 amplicon attractor; and ESR1, SCUBE2, PGR to the ESR1 attractor.

In contrast, the present invention relates, in certain embodiments, to a “multidimensional” biomarker product that will be applicable to multiple cancer types. Each of the dimensions of such embodiments will correspond to a specific attractor detected from a sharp choice of the gene or other feature at its core, reflecting a precise biological attribute of cancer. For example, each relevant amplicon can be identified by the coordinate co-expression of the top few genes of the attractor without any need for sequencing, and each will correspond to another dimension. The collection of the independent results in many dimensions will provide a clearer diagnostic and prognostic image after cleanly distinguishing the contributions of each component, whether the embodiment is directed to cancer or any other indication. Thus, even though molecular marker genes in existing products are often separated into groups that are related to the attractor designation, the improvement in diagnostic, prognostic, or predictive accuracy resulting from better such group designation and better choice of genes in each group that is achieved using the methods and compositions described herein is highly desirable.

### 4.3.2. Methods of Using Attractor Molecular Signatures for Diagnosis and/or Treatment

In certain embodiments, the present invention provides for methods of treating a subject, such as, but not limited to, methods comprising performing a diagnostic method as set forth herein and then, if an attractor metagene is detected in a sample of the subject, administering therapy consistent with the presence or absence of the attractor molecular signature. In certain embodiments, the combinations of attractor molecular signatures can be detected, alone or in combination with other features (e.g., expression levels of specific genes, information as to tumor size, and number of positive lymph nodes). In certain embodiments such methods can comprise the use of one or more (including all) of the following eleven features FGD3-SUSD3, CIN, MES*, LYM, END, LYM*, CD68, DNAJB9, CXCL12, number of positive lymph nodes, and tumor size.

In certain non-limiting embodiments of the present invention, a diagnostic method as set forth above is performed and a therapeutic decision is made in light of the results of that diagnostic method. For example, but not by way of limitation, a therapeutic decision, such as whether to prescribe a particular therapeutic or class of therapeutic can be made in light of the results of a diagnostic method as set forth below. The results of the diagnostic methods described herein are relevant to the therapeutic decision as the presence of the attractor molecular signature or a subset of features associated with it, in a sample from a subject can, in certain embodiments, indicate a decrease in the relative benefit conferred by a particular therapeutic intervention.

In certain embodiments, a diagnostic method as set forth below is performed and a decision regarding whether to continue a particular therapeutic regimen is made in light of the results of that diagnostic method. For example, but not by way of limitation, a decision whether to continue a particular therapeutic regimen, such as whether to continue with one or more of the therapeutics described herein can be made in light of the results of a diagnostic method as set forth below. The results of the diagnostic method are relevant to the decision whether to continue a particular therapeutic regimen as the presence of the attractor molecular signature or a subset of features associated with it, in a sample from a subject can be indicative of the subject's responsiveness to the particular therapeutic. In certain embodiments, the combinations of attractor molecular signatures can be detected, alone or in combination with other features (e.g., expression levels of specific genes, information as to tumor size, and number of positive lymph nodes). In certain embodiments such methods can comprise the use of one or more (including all) of the following eleven features FGD3-SUSD3, CIN, MES*, LYM, END, LYM*, CD68, DNAJB9, CXCL12, number of positive lymph nodes, and tumor size.

In certain embodiments, the present invention provides for methods of performing a prognosis of a subject identified as having cancer, such as, but not limited to, methods comprising performance of a diagnostic method as set forth herein (e.g., obtaining a sample from the subject and determining whether an attractor molecular signature can be detected in the sample) and then, if an attractor molecular signature is detected in a sample of the subject, predicting the likely outcome (i.e., performing a prognosis) of the cancer, e.g., the likely survival duration. In certain embodiments, the prognosis will be based on the presence of one or more attractor molecular signature. In certain embodiments, the prognosis will be based on the presence of one or more attractor molecular signature and one or more additional factors, such as clinical and molecular features (e.g., the number of cancer-positive lymph nodes, age at diagnosis, and expression levels of particular genes exhibiting protective activity). In certain embodiments, the combinations of attractor molecular signatures can be detected, alone or in combination with other features (e.g., expression levels of specific genes, information as to tumor size, and number of positive lymph nodes). In certain embodiments such methods can comprise the use of one or more (including all) of the following eleven features FGD3-SUSD3, CIN, MES*, LYM, END, LYM*, CD68, DNAJB9, CXCL12, number of positive lymph nodes, and tumor size.

In certain embodiments, biomarker assays capable of identifying a attractor molecular signatures in patient samples for use in connection with the therapeutic interventions discussed herein can include, but are not limited to, nucleic acid amplification assays; nucleic acid hybridization assays; as well as protein detection assays that are specific for the attractor molecular signature biomarkers or “features” discussed herein. In certain embodiments, the assays of the present invention involve combinations of such detection techniques, e.g., but not limited to: assays that employ both amplification and hybridization to detect a change in the expression, such as overexpression or decreased expression, of a gene at the nucleic acid level; immunoassays that detect a change in the expression of a gene at the protein level; as well as combination assays comprising a nucleic acid-based detection step and a protein-based detection step.

A “sample” from a subject to be tested according to one of the assay methods described herein can be at least a portion of a tissue, at least a portion of a tumor, a cell, a collection of cells, or a fluid (e.g., blood, cerebrospinal fluid, urine, expressed prostatic fluid, peritoneal fluid, a pleural effusion, peritoneal fluid, etc.). In certain embodiments the sample used in connection with the assays of the instant invention will be obtained via a biopsy. Biopsy can be done by an open or percutaneous technique. Open biopsy is conventionally performed with a scalpel and can involve removal of the entire tumor mass (excisional biopsy) or a part of the tumor mass (incisional biopsy). Percutaneous biopsy, in contrast, is commonly performed with a needle-like instrument either blindly or with the aid of an imaging device, and can be either a fine needle aspiration (FNA) or a core biopsy. In FNA biopsy, individual cells or clusters of cells are obtained for cytologic examination. In core biopsy, a core or fragment of tissue is obtained for histologic examination which can be done via a frozen section or paraffin section.

“Overexpression” and “increased activity”, as used herein, refers to an increase in expression or activity, respectively, of a gene product relative to a normal or control value, which, in non-limiting embodiments, is an increase of at least about 30% or at least about 40% or at least about 50%, or at least about 100%, or at least about 200%, or at least about 300%, or at least about 400%, or at least about 500%, or at least 1000%.

“Decreased expression” and “decreased activity”, as used herein, refers to an decrease in expression or activity, respectively, of a gene product relative to a normal or control value, which, in non-limiting embodiments, is an decrease of at least about 30% or at least about 40% or at least about 50%, at least about 90%, or a decrease to a level where the expression or activity is essentially undetectable using conventional methods.

As used herein, a “gene product” refers to any product of transcription and/or translation of a gene. Accordingly, gene products include, but are not limited to, microRNA, pre-mRNA, mRNA, and proteins.

In certain embodiments, the present invention provides compositions and methods for the detection of gene expression indicative of all or part of the attractor molecular signature in a sample using nucleic acid hybridization and/or amplification-based assays.

In non-limiting embodiments, the genes/proteins within the attractor molecular signature set forth above constitute at least 10 percent, or at least 20 percent, or at least 30 percent, or at least 40 percent, or at least 50 percent, or at least 60 percent, or at least 70 percent, or at least 80 percent, or at least 90 percent, of the genes/proteins being evaluated in a given assay.

In certain embodiments, the present invention provides compositions and methods for the detection of the particular features (e.g., gene or miRNA sequence, and/or methylation state) indicative of all or part of the attractor molecular signature in a sample using a nucleic acid hybridization and/or amplification assay, wherein nucleic acid from said sample, or amplification products thereof, are hybridized to an array of one or more nucleic acid probe sequences. In certain embodiments, an “array” comprises a support, preferably solid, with one or more nucleic acid probes attached to the support. Preferred arrays typically comprise a plurality of different nucleic acid probes that are coupled to a surface of a substrate in different, known locations. These arrays, also described as “microarrays” or “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991).

Arrays can generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes.

Although a planar array surface is preferred, the array can be fabricated on a surface of virtually any shape or even a multiplicity of surfaces. Arrays can be nucleic acids on beads, gels, polymeric surfaces, fibers such as fiber optics, glass or any other appropriate substrate. See U.S. Pat. Nos. 5,770,358, 5,789,162, 5,708,153, 6,040,193 and 5,800,992.

In certain embodiments, the arrays of the present invention can be packaged in such a manner as to allow for diagnostic, prognostic, and/or predictive use or can be an all-inclusive device; e.g., U.S. Pat. Nos. 5,856,174 and 5,922,591.

In certain embodiments, the hybridization assays of the present invention comprise a primer extension step. Methods for extension of primers from solid supports have been disclosed, for example, in U.S. Pat. Nos. 5,547,839 and 6,770,751. In addition, methods for genotyping a sample using primer extension have been disclosed, for example, in U.S. Pat. Nos. 5,888,819 and 5,981,176.

In certain embodiments, the methods for detection of all or a part of the attractor molecular signature in a sample involves a nucleic acid amplification-based assay. In certain embodiments, such assays include, but are not limited to: real-time PCR (for example see Mackay, Clin. Microbiol. Infect. 10(3):190-212, 2004), Strand Displacement Amplification (SDA) (for example see Jolley and Nasir, Comb. Chem. High Throughput Screen. 6(3):235-44, 2003), self-sustained sequence replication reaction (3SR) (for example see Mueller et al., Histochem. Cell. Biol. 108(4-5):431-7, 1997), ligase chain reaction (LCR) (for example see Laffler et al., Ann. Biol. Clin. (Paris).51(9):821-6, 1993), transcription mediated amplification (TMA) (for example see Prince et al., J. Viral Hepat. 11(3):236-42, 2004), or nucleic acid sequence based amplification (NASBA) (for example see Romano et al., Clin. Lab. Med. 16(1):89-103, 1996).

In certain embodiments of the present invention, a PCR-based assay, such as, but not limited to, real time PCR is used to detect the presence of an attractor molecular signature in a test sample. In certain embodiments, attractor metagene-specific PCR primer sets are used to amplify attractor molecular signature-associated RNA and/or DNA targets. Signal for such targets can be generated, for example, with fluorescence-labeled probes. In the absence of such target sequences, the fluorescence emission of the fluorophore can be, in certain embodiments, eliminated by a quenching molecule also operably linked to the probe nucleic acid. However, in the presence of the target sequences, probe binds to template strand during primer extension step and the nuclease activity of the polymerase catalyzing the primer extension step results in the release of the fluorophore and production of a detectable signal as the fluorophore is no longer linked to the quenching molecule. (Reviewed in Bustin, J. Mol. Endocrinol 25, 169-193(2000)). The choice of fluorophore (e.g., FAM, TET, or Cy5) and corresponding quenching molecule (e.g. BHQ1 or BHQ2) is well within the skill of one in the art and specific labeling kits are commercially available.

In certain embodiments, the present invention provides compositions and methods for the detection of gene expression indicative of all or part of the attractor molecular signature in a sample by employing high throughput sequencing techniques, such as RNA-seq. (See, e.g., Wang et al., RNA-Seq: a revolutionary tool for transcriptomics, Nat Rev Genet. 2009 January; 10(1): 57-63). In general, such techniques involve obtaining a sample population of RNA (total or fractionated, such as poly(A)+) which is then converted to a library of cDNA fragments, typically of 30-400 bp in length. These cDNA fragments will be generated to include adaptors attached to one or both ends, depending on whether the subsequent sequencing step proceeds from one or both ends. Each of the adaptor-tagged molecules, with or without amplification, can then be sequenced in a high-throughput manner to obtain short sequences. Virtually any high-throughput sequencing technology can be used for the sequencing step, including, but not limited to the Illumina IG®, Applied Biosystems SOLiD®, Roche 454 Life Science®, and Helicos Biosciences tSMS® systems. Following sequencing, bioinformatics techniques can be used to either align there results against a reference genome or to assemble the results de novo. Such analysis is capable of identifying both the level of expression for each gene as well as the sequence of particular expressed genes.

In certain embodiments, the present invention provides compositions and methods for the detection of protein expression indicative of all or part of the attractor molecular signature in a sample by detecting changes in concentration of the protein, or proteins, encoded by the genes of interest.

In certain embodiments, the present invention relates to the use of immunoassays to detect modulation of protein expression by detecting changes in the concentration of proteins expressed by a gene of interest. Numerous techniques are known in the art for detecting changes in protein expression via immunoassays. (See The Immunoassay Handbook, 2nd Edition, edited by David Wild, Nature Publishing Group, London 2001.) In certain of such immunoassays, antibody reagents capable of specifically interacting with a protein of interest, e.g., an individual member of the attractor metagene, are covalently or non-covalently attached to a solid phase. Linking agents for covalent attachment are known and can be part of the solid phase or derivatized to it prior to coating. Examples of solid phases used in immunoassays are porous and non-porous materials, latex particles, magnetic particles, microparticles, strips, beads, membranes, microtiter wells and plastic tubes. The choice of solid phase material and method of labeling the antibody reagent are determined based upon desired assay format performance characteristics. For some immunoassays, no label is required, however in certain embodiments, the antibody reagent used in an immunoassay is attached to a signal-generating compound or “label”. This signal-generating compound or “label” is in itself detectable or can be reacted with one or more additional compounds to generate a detectable product (see also U.S. Pat. No. 6,395,472 B1). Examples of such signal generating compounds include chromogens, radioisotopes (e.g., 1251, 1311, 32P, 3H, 35S, and 14C), fluorescent compounds (e.g., fluorescein and rhodamine), chemiluminescent compounds, particles (visible or fluorescent), nucleic acids, complexing agents, or catalysts such as enzymes (e.g., alkaline phosphatase, acid phosphatase, horseradish peroxidase, beta-galactosidase, and ribonuclease). In the case of enzyme use, addition of chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful in the context of the methods of the present invention.

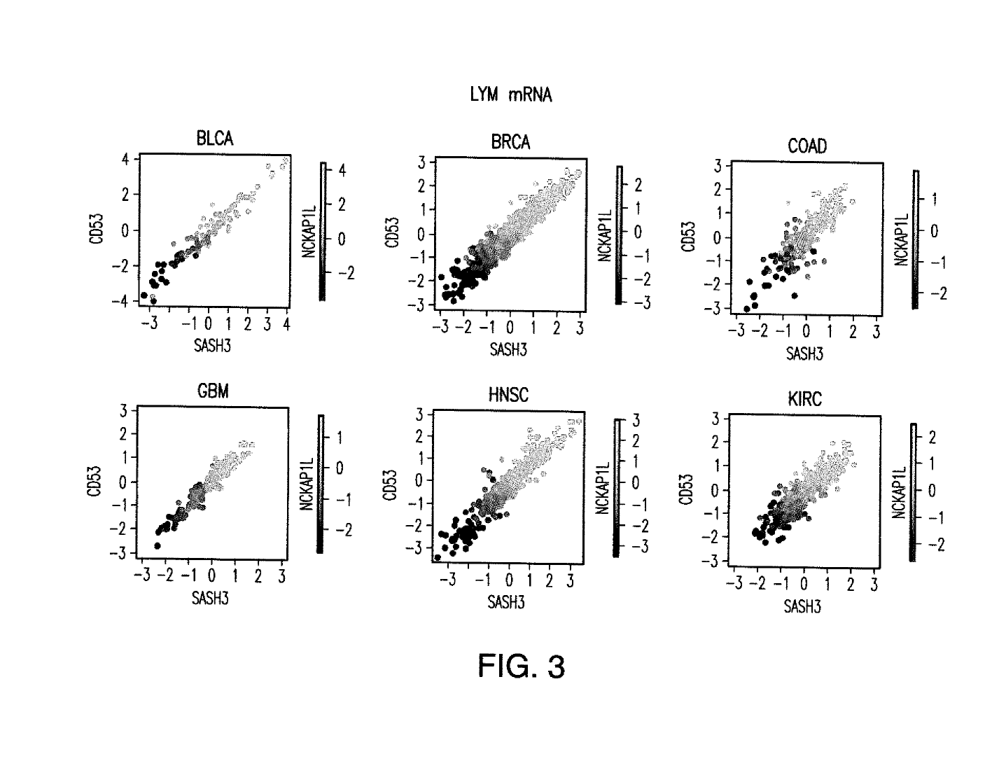

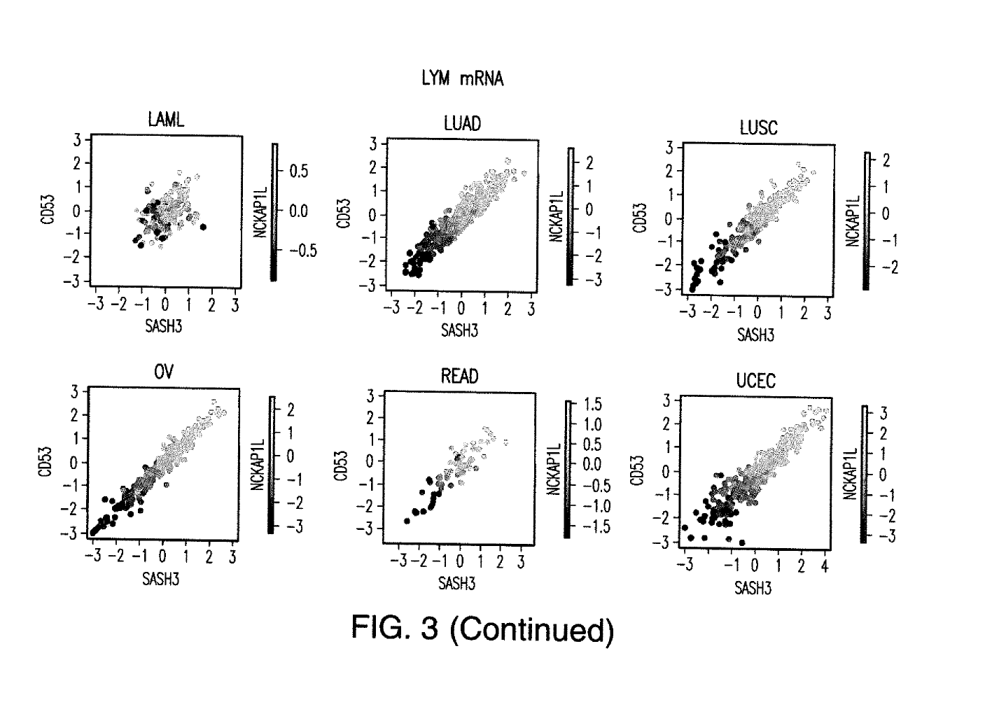

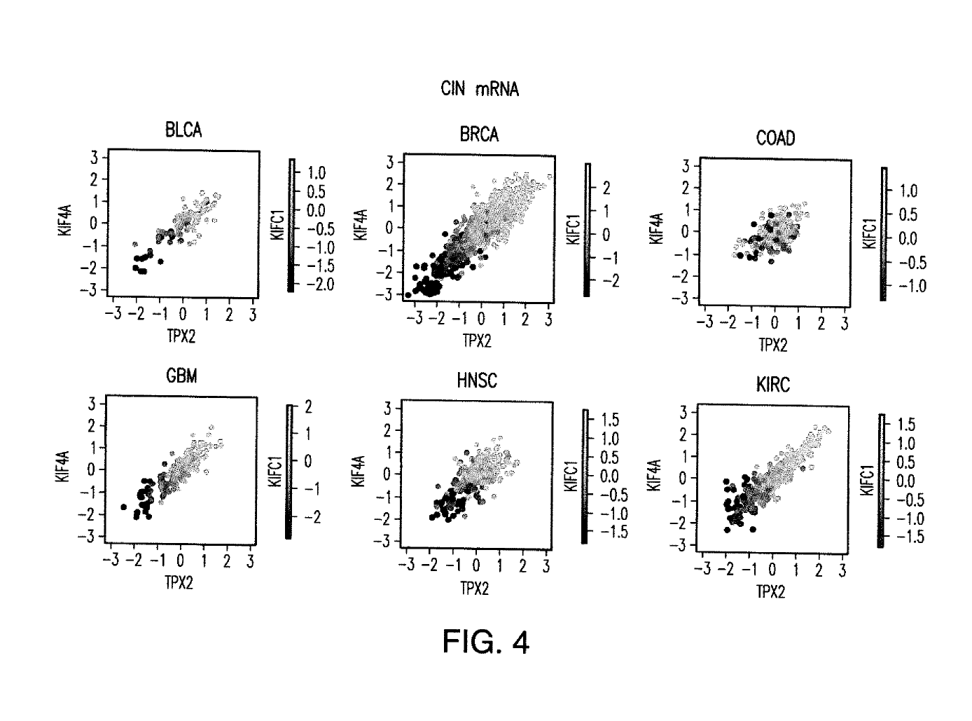

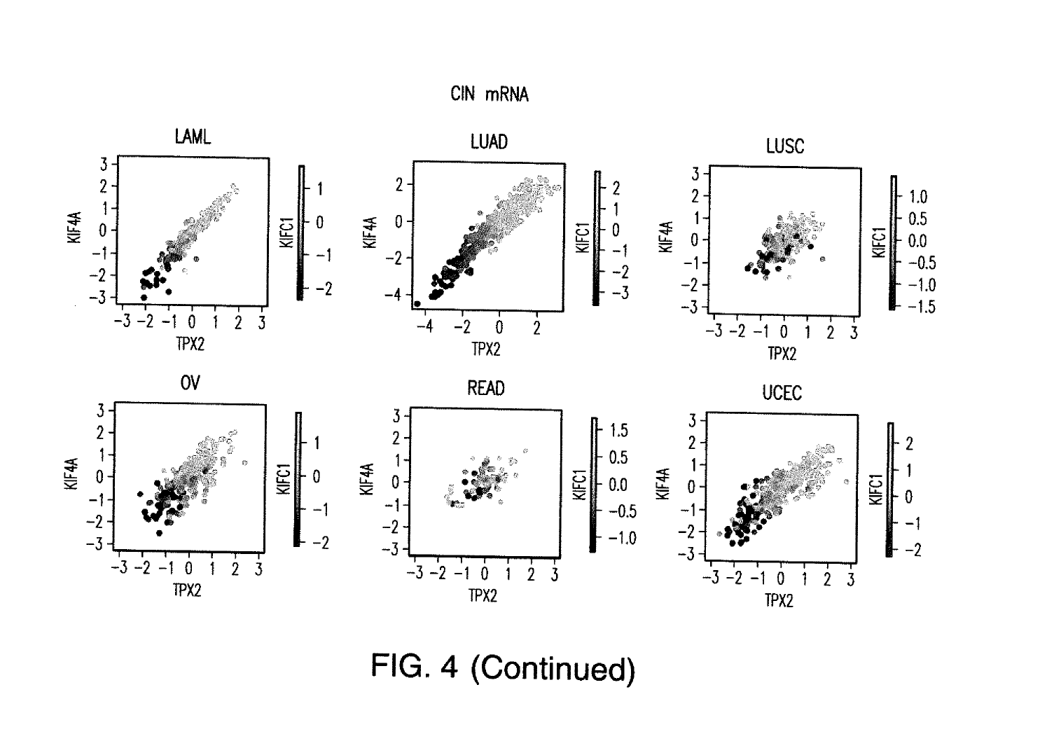

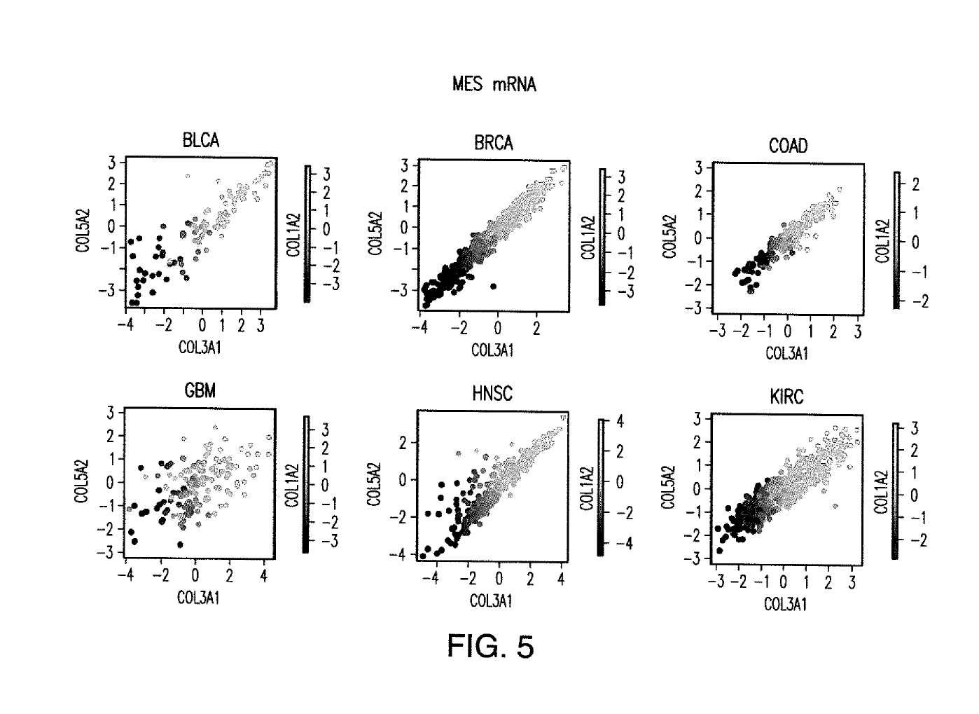

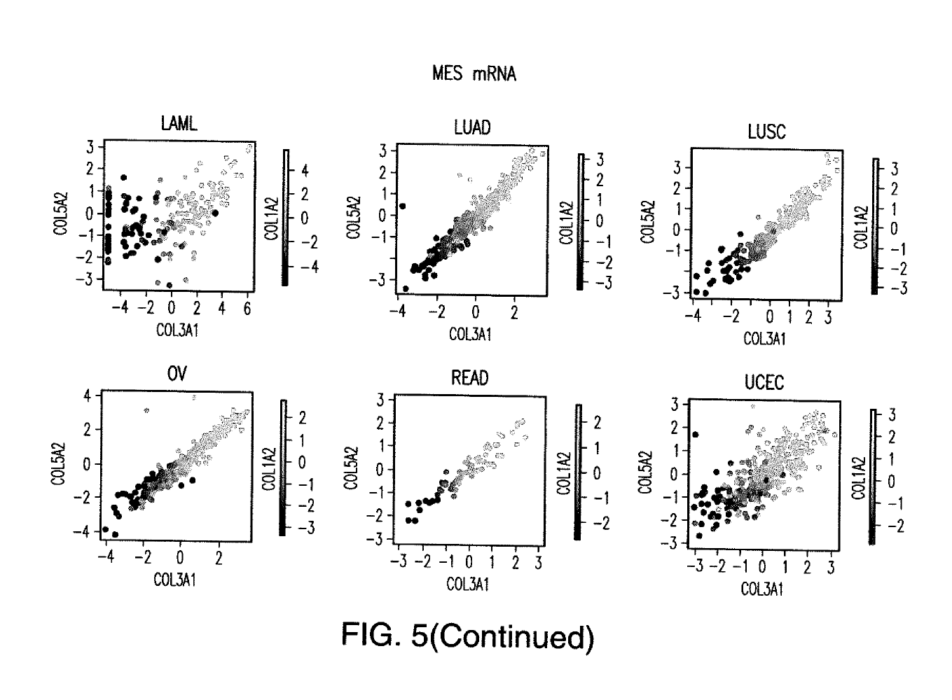

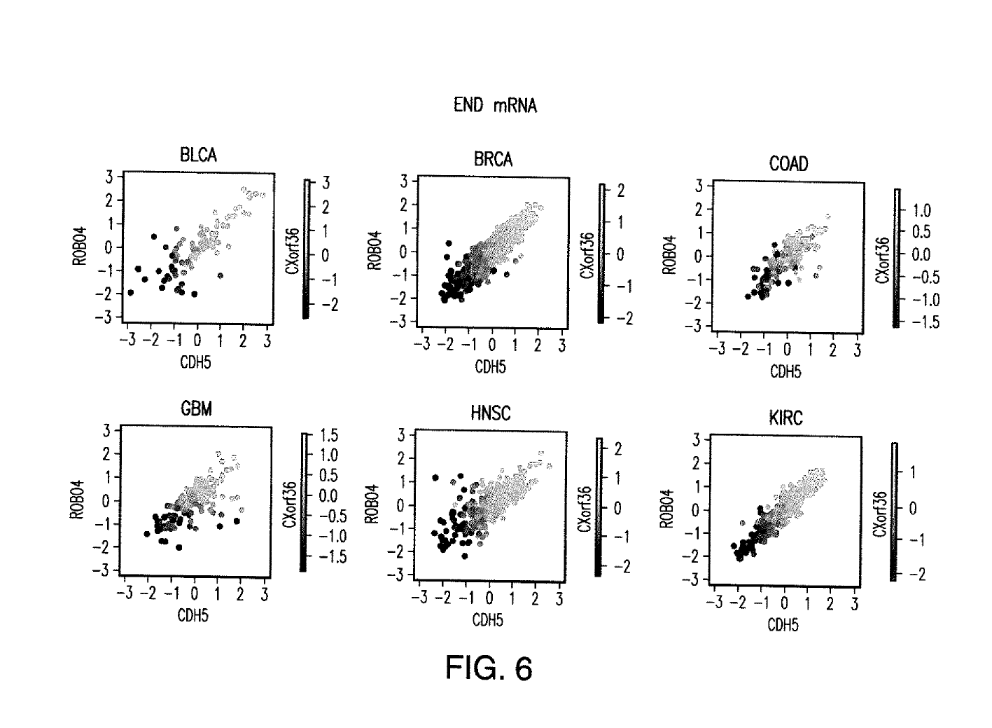

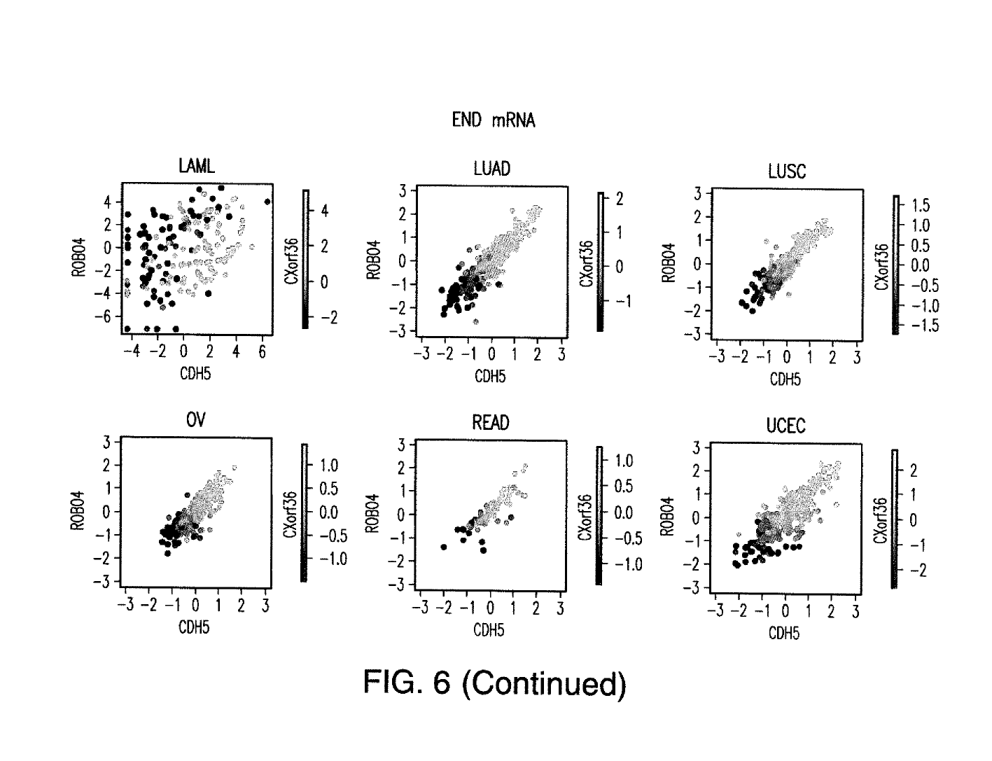

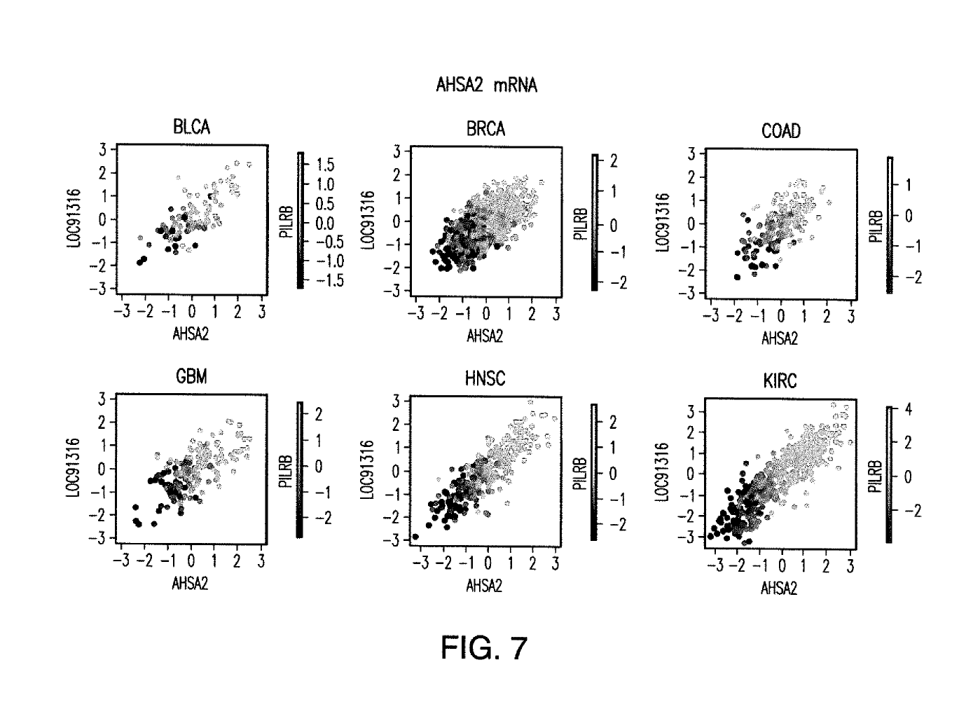

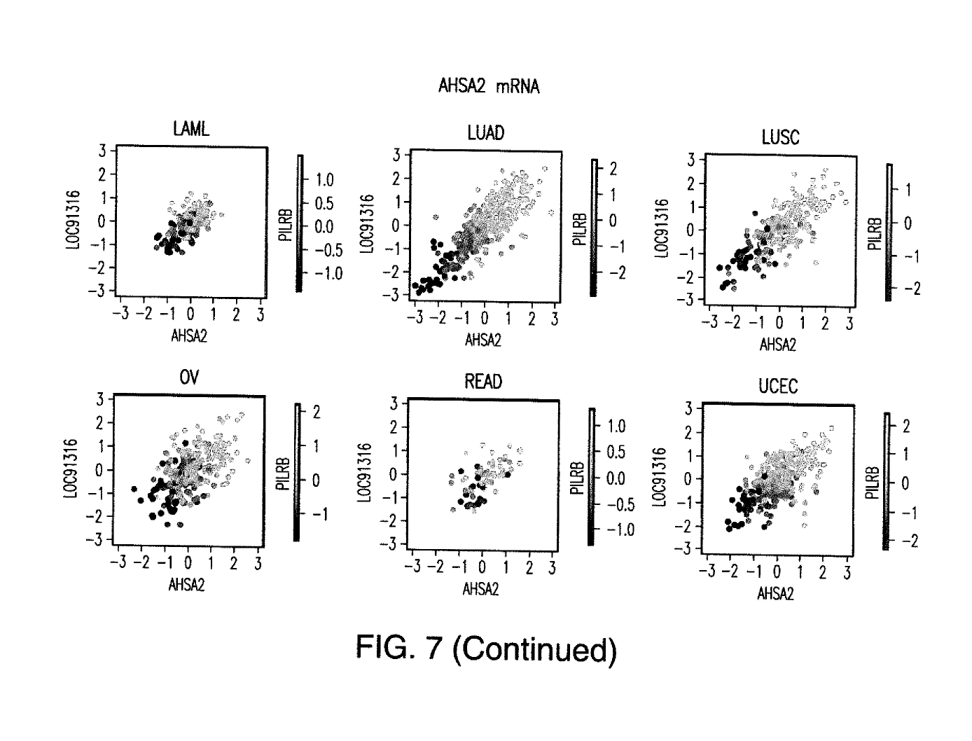

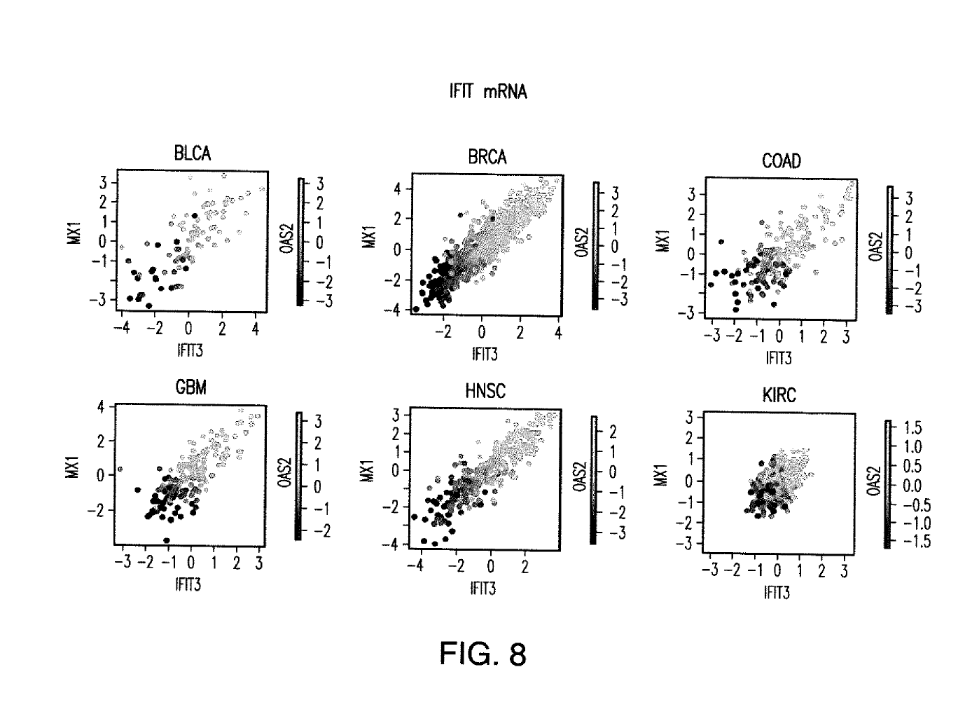

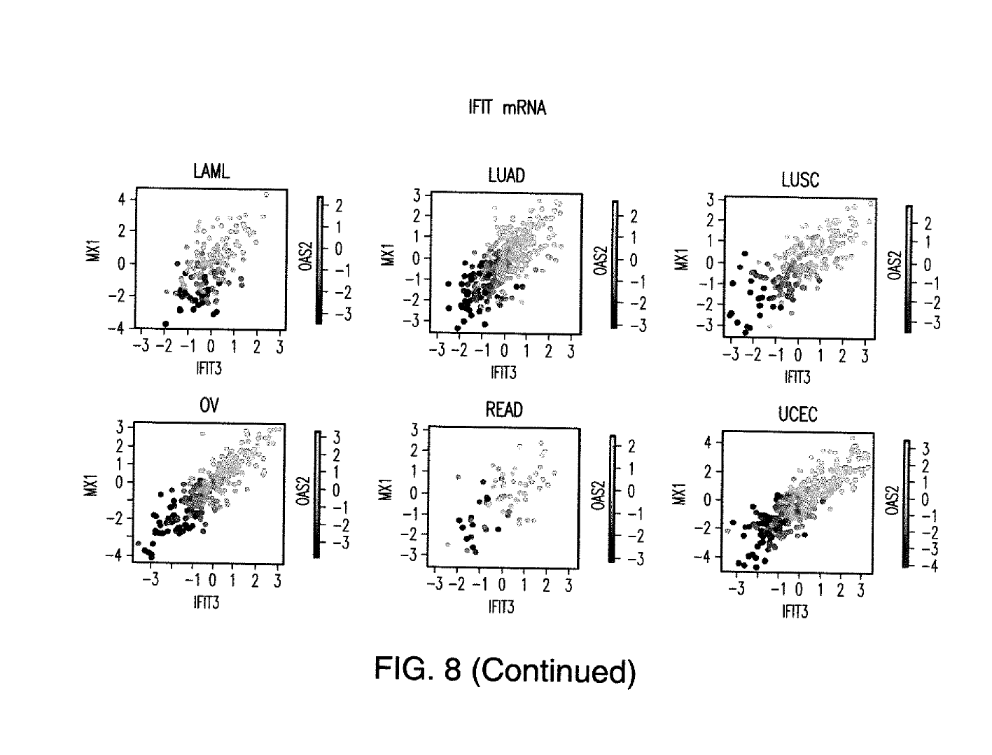

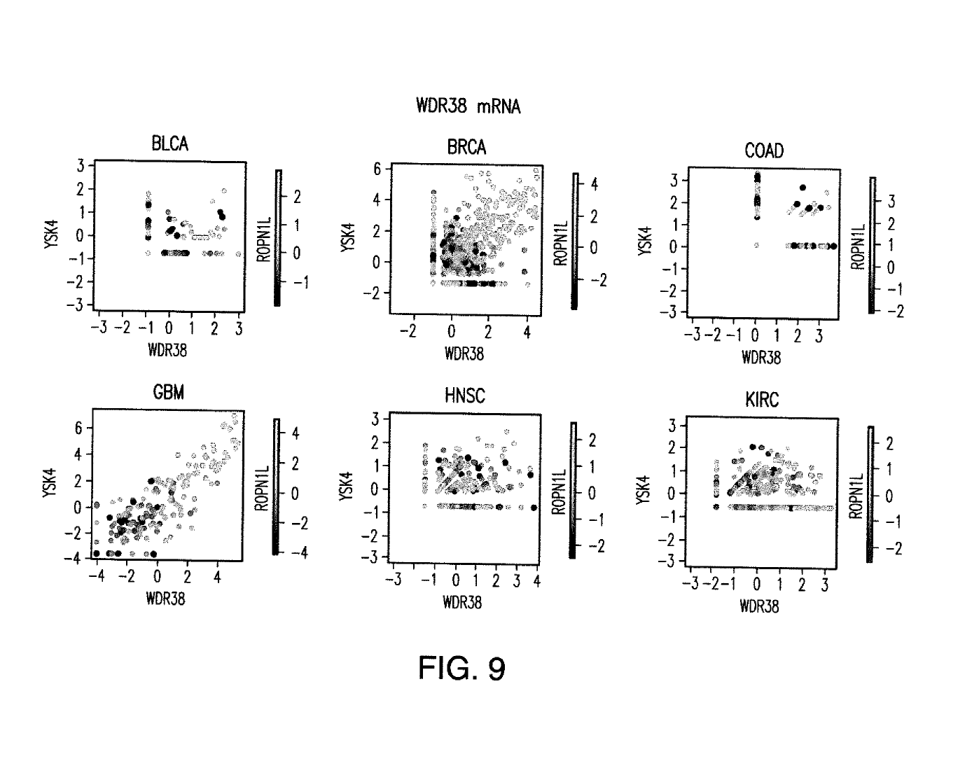

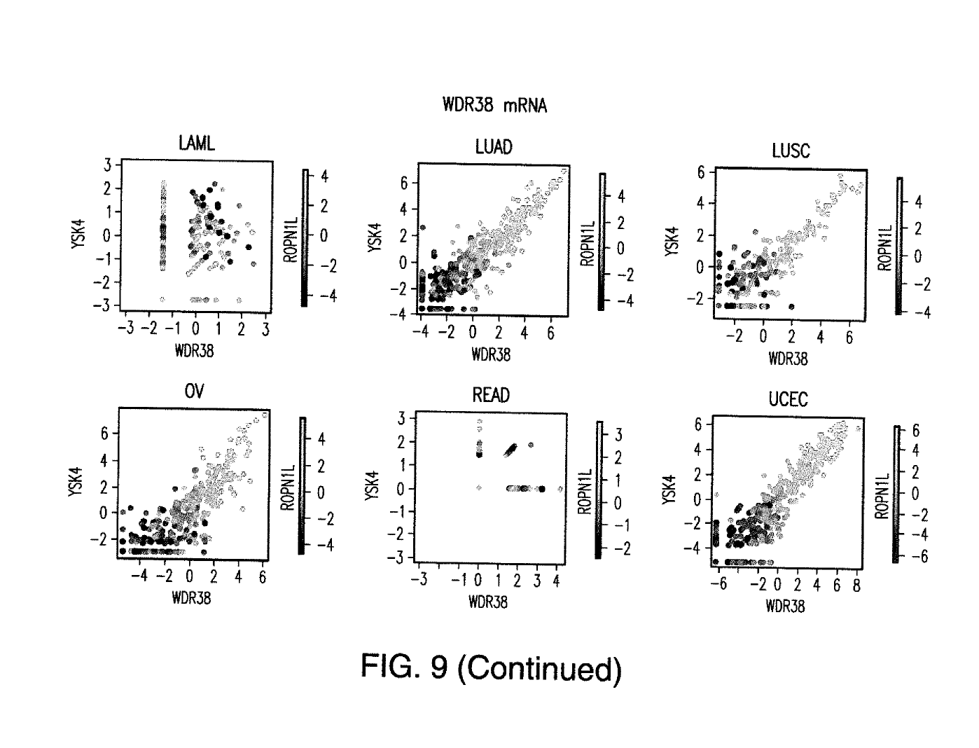

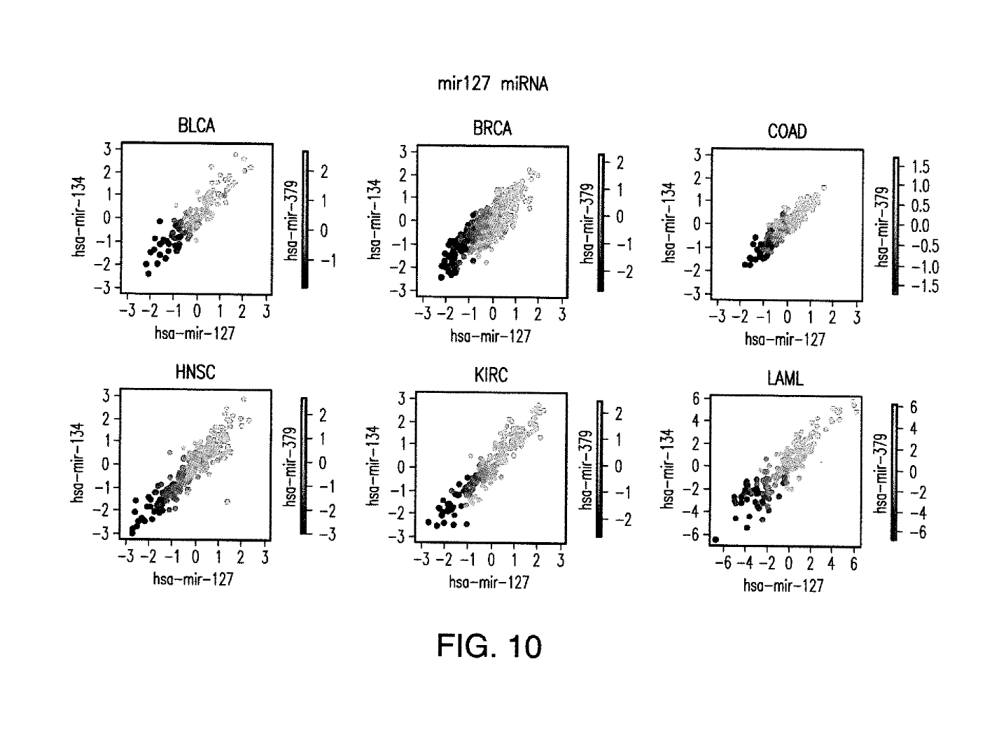

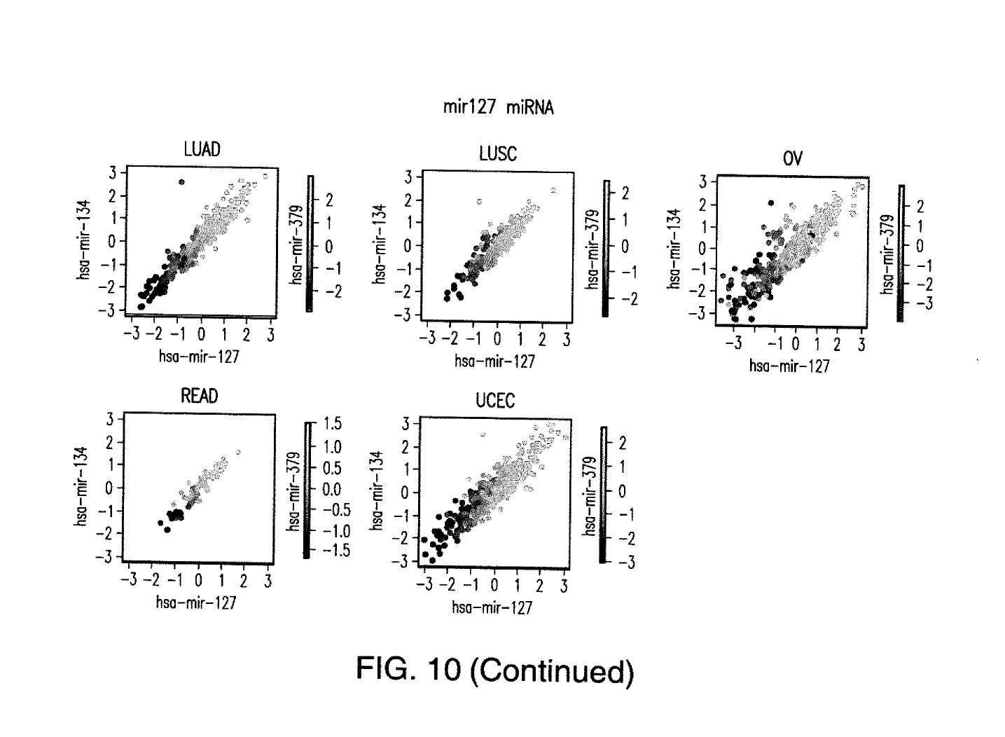

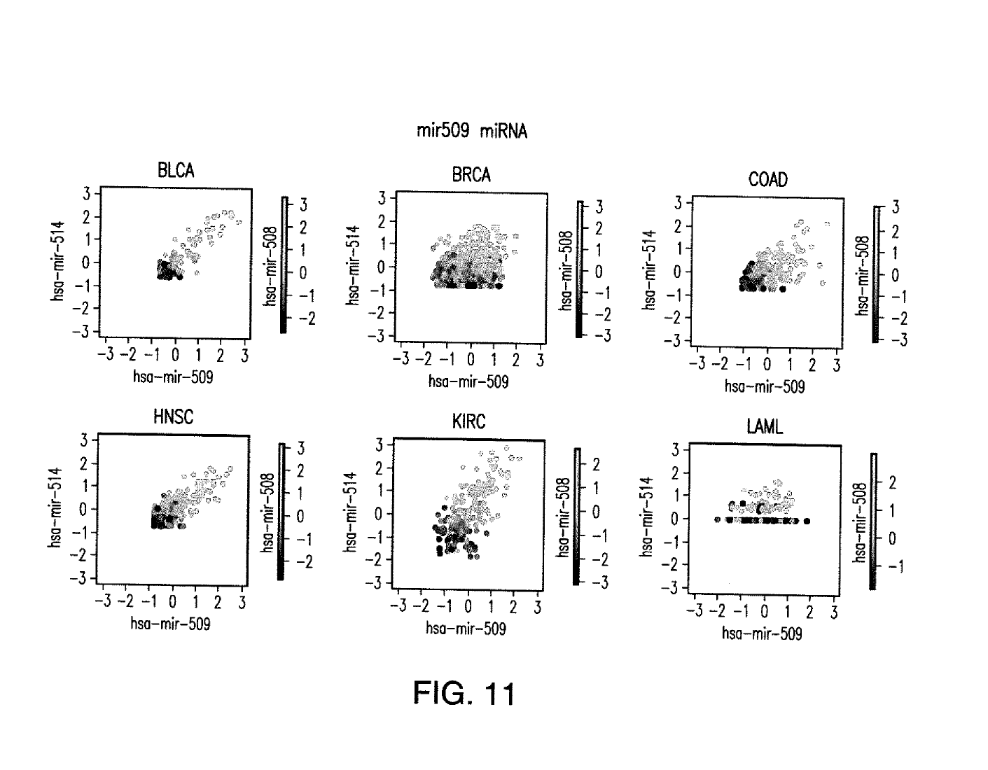

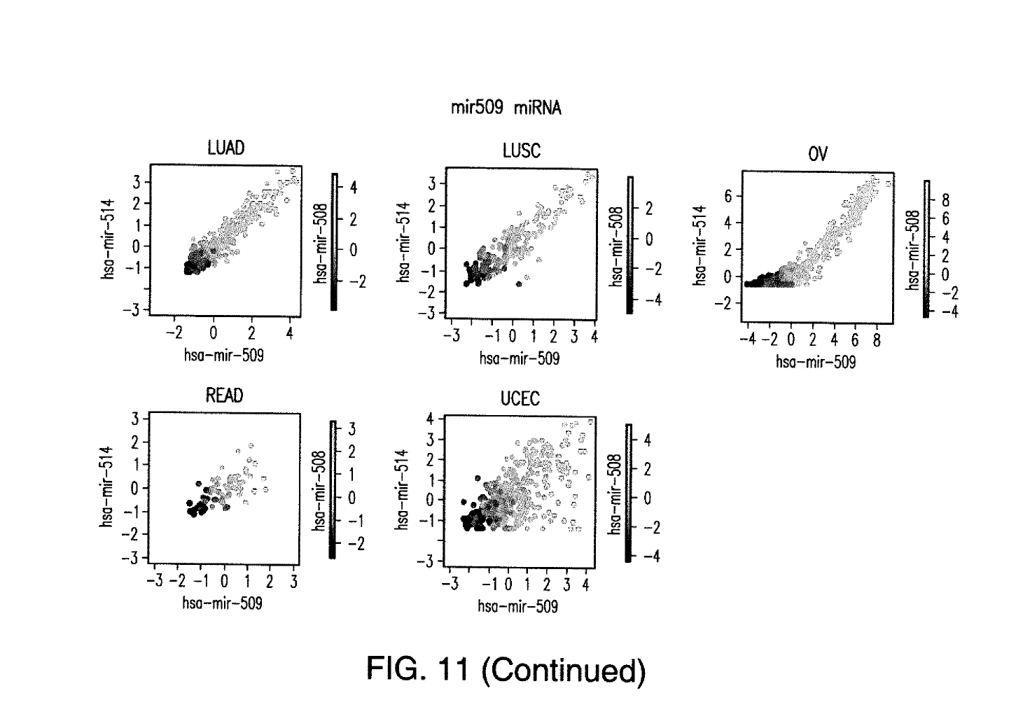

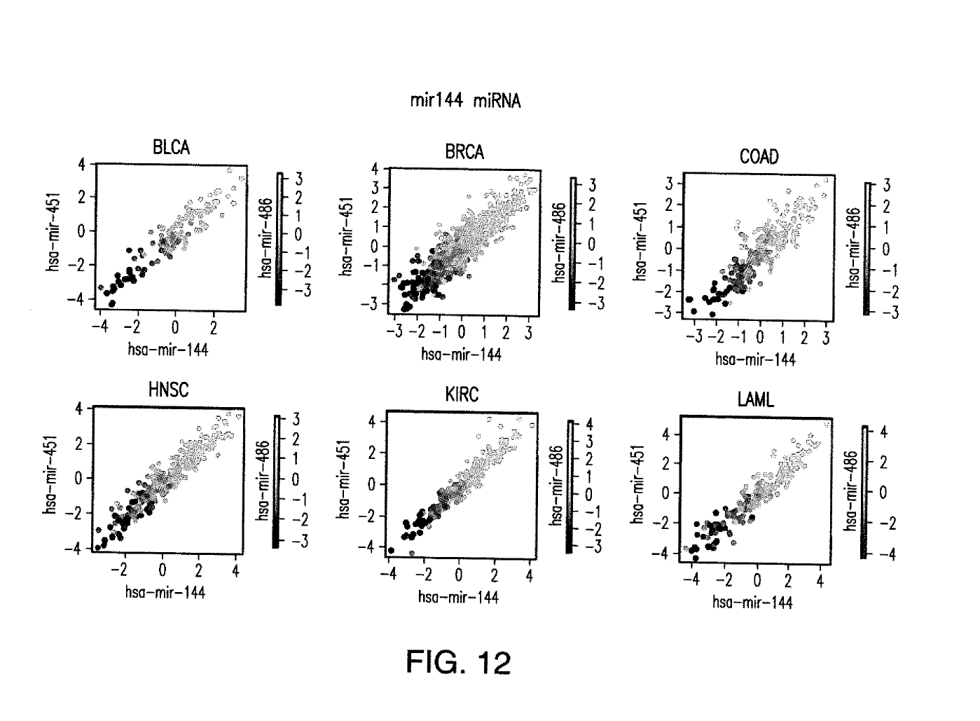

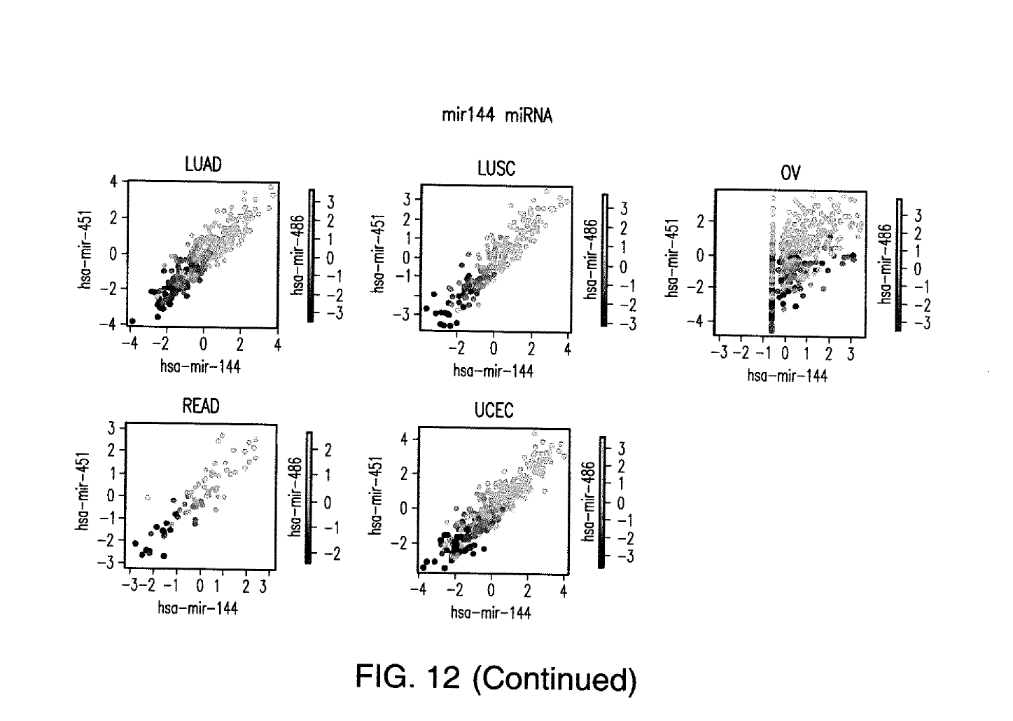

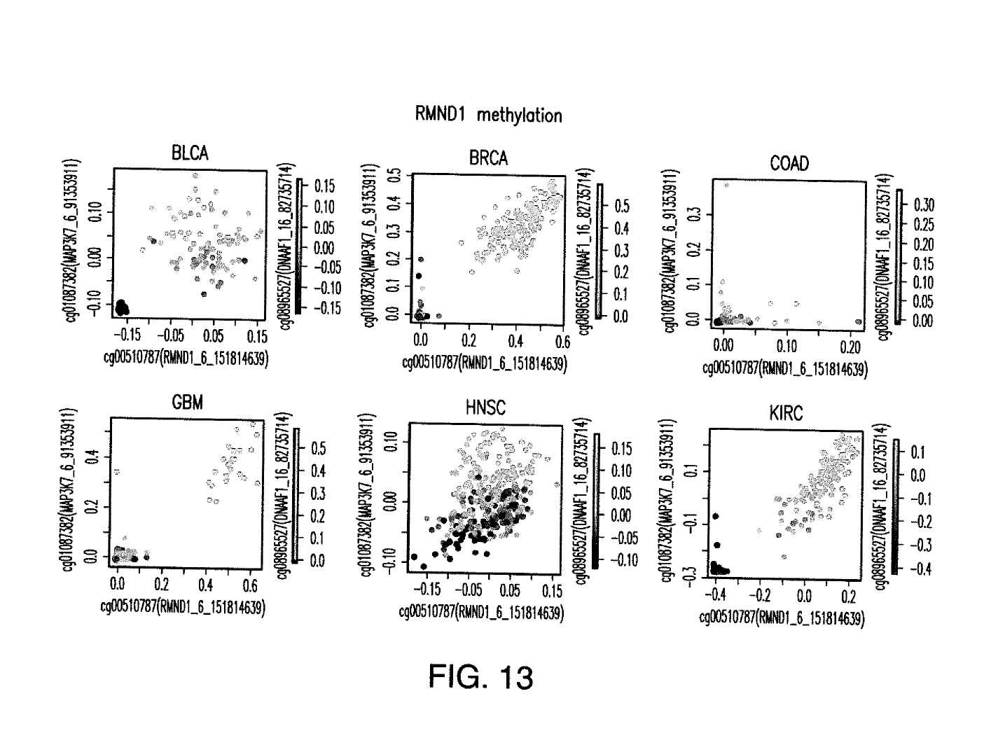

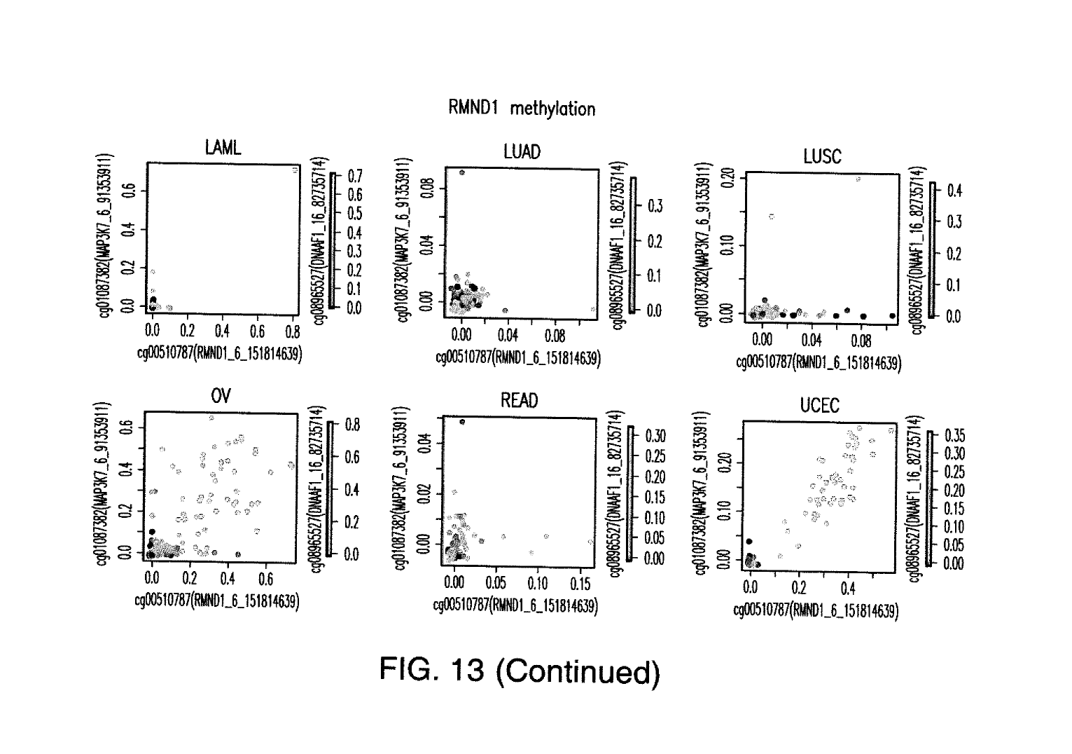

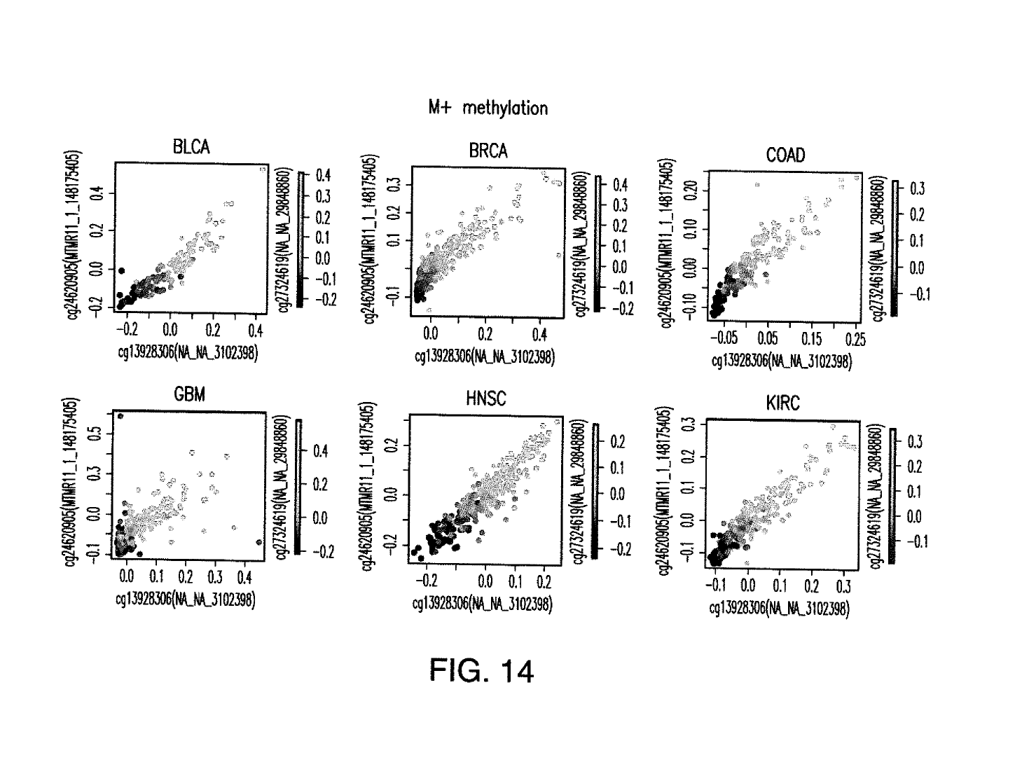

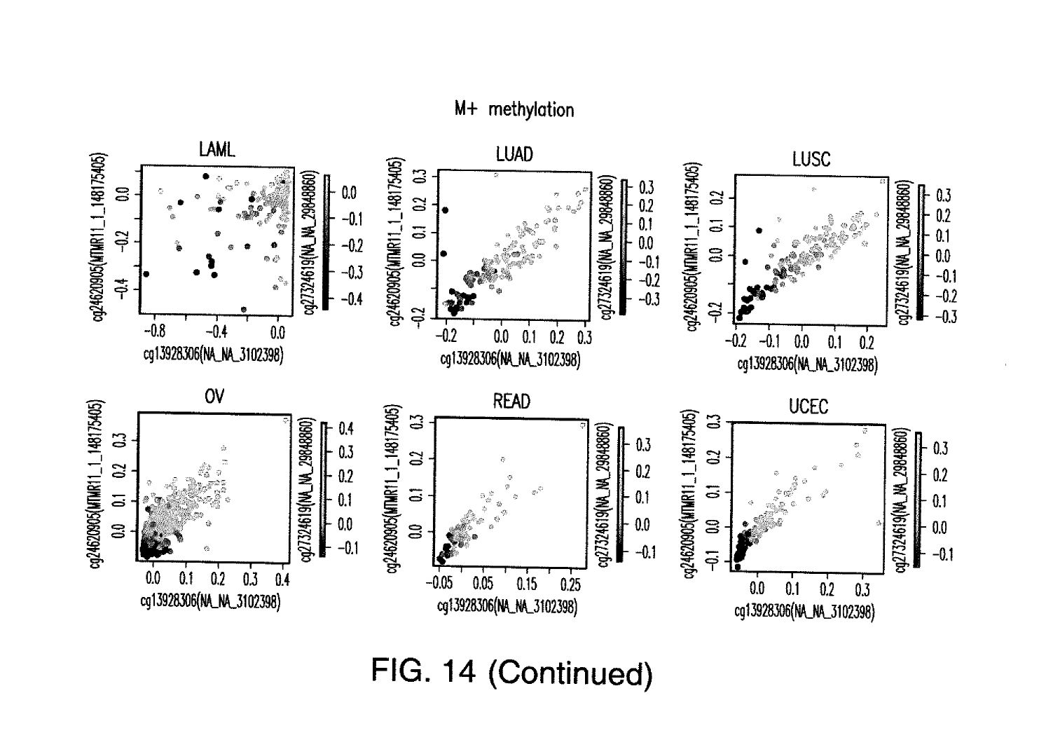

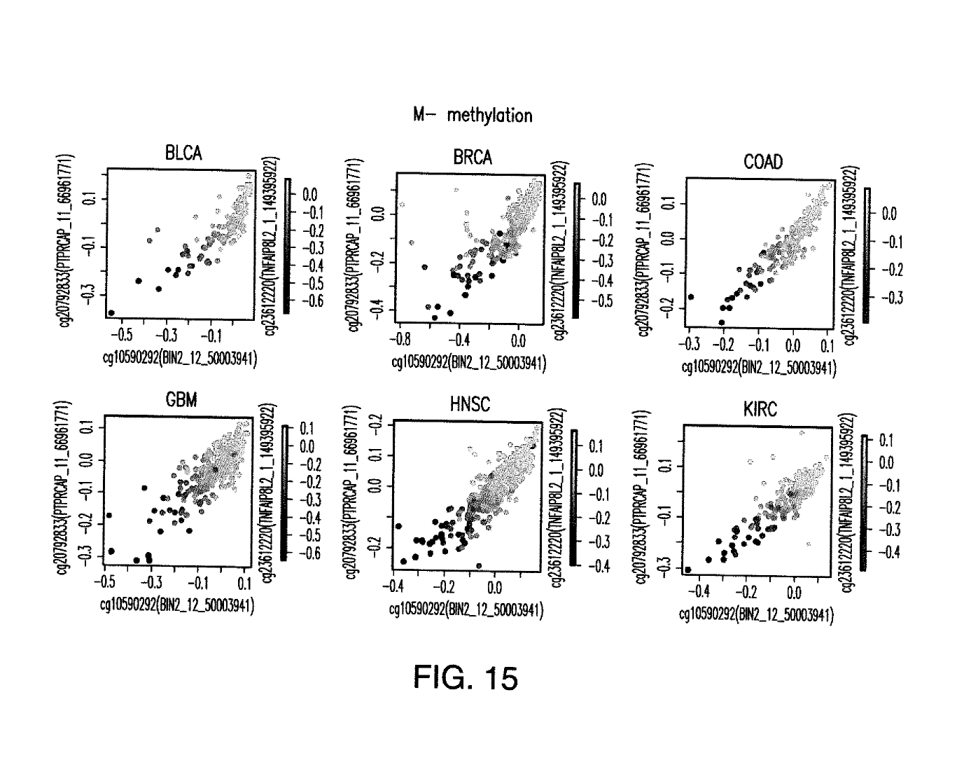

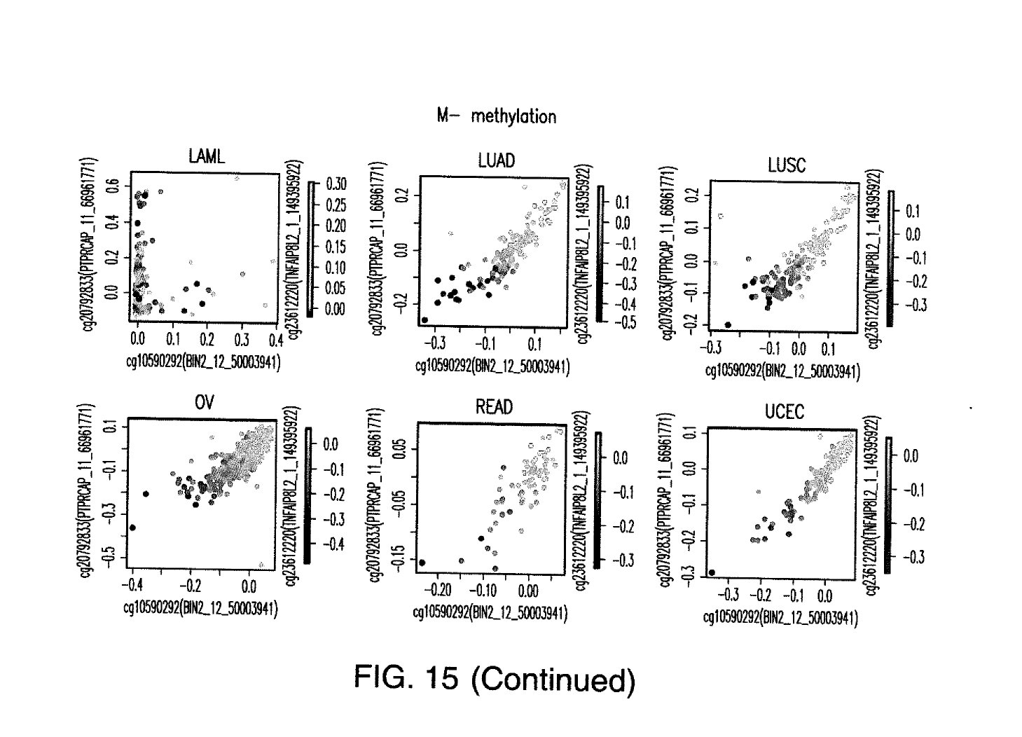

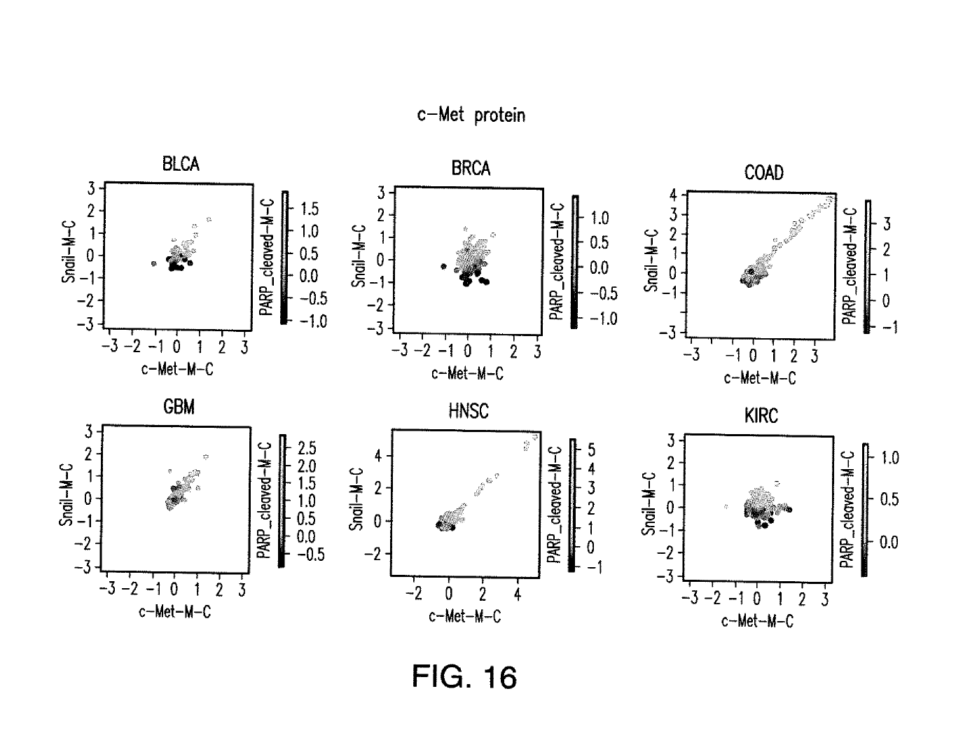

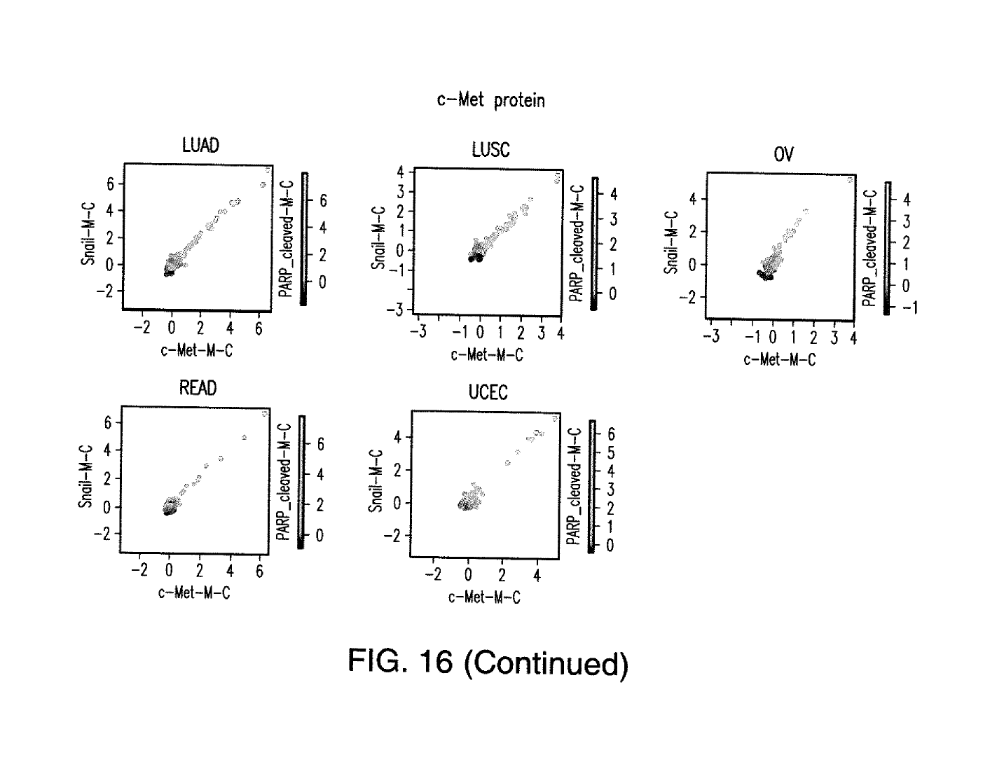

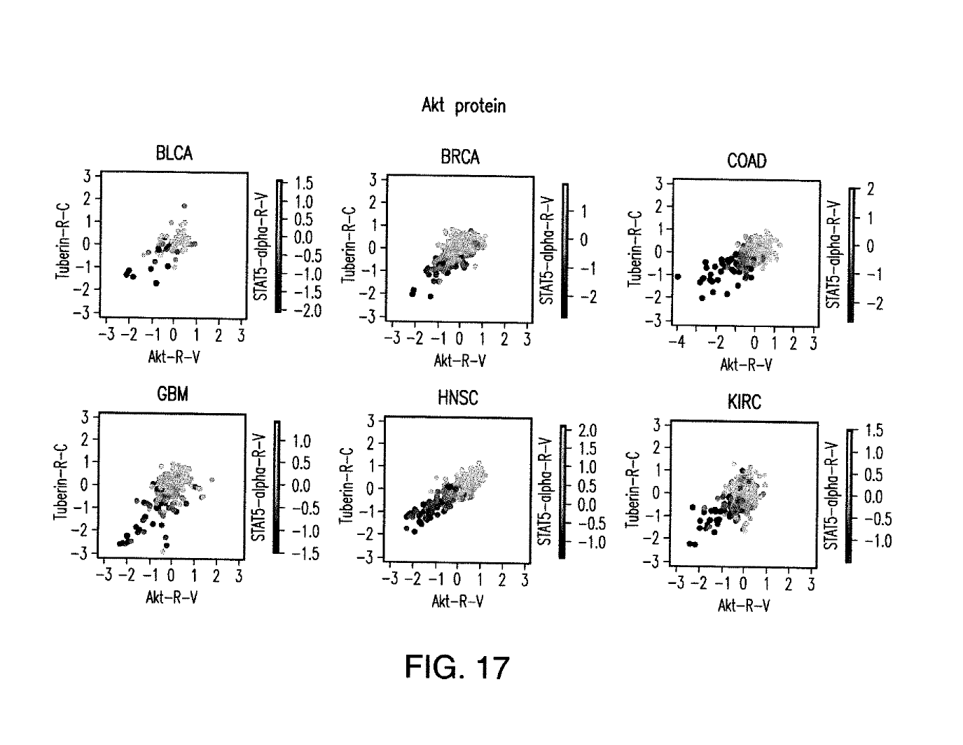

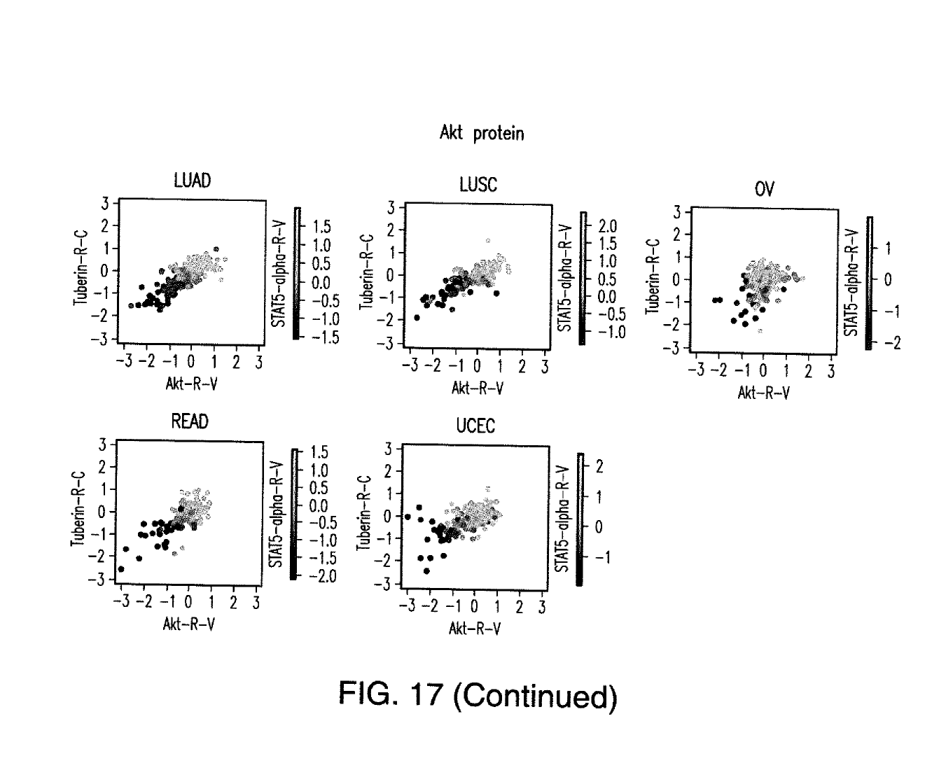

In certain embodiments, the assays of the present invention are capable of detecting coordinated modulation of expression, for example, but not limited to, overexpression, of the genes associated with the attractor molecular signature. In certain embodiments, such detection involves, but is not limited to, detection of the expression of one or more of the attractor molecular signature identified in FIGS. 3-17, 19A-D, 20A-D, and 21A-D.

In certain embodiments, the present invention provides compositions and methods for the detection of methylation state of all or part of an attractor molecular signature in a sample by detecting changes in methylation state of the genes of interest. For example, by not by way of limitation, the methylation state of a gene of interest can be determined by processes known in the art to separate and detect methylated from unmethylated nucleic acids, e.g., DNA, through immunoprecipitation of methylated DNA (MeDIP) (Mohn et al., Methods in Molecular Biology, 507:55-64 (2009)), methylation specific binding protein columns, methylation-sensitive restriction digestion, and/or methylation-specific PCR (U.S. Patent Publication 20130116409; 9. Das et al., Computational prediction of methylation status in human genomic sequences, PNAS 103(28):10713-10716 (2006); Hendrich et al., Identification and Characterization of a Family of Mammalian Methyl-CpG Binding Proteins, Mol Cell Biol. 18(11): 6538-6547 (1998); Frommer et al., A genomic sequencing protocol that yields a positive display of 5-methylcytosine residues in individual DNA strands, Proc Natl Acad Sci USA, 89:1827-183 (1992); and Xiong et al., COBRA: a sensitive and quantitative DNA methylation assay, Nucleic Acids Research, 25(12):2532-2534 (1997). Additional techniques for the detection of a methylation state of a gene of interest include nanopore-based detection systems, such as those described in U.S. Pat. No. 8,394,584.

In certain embodiments, methylation-specific PCR is employed to detect the methylation state of a gene of interest. Methylation-specific PCR relies on a pre-amplification bisulfite treatment, where any unmethylated cytosine residue is deaminated thereby converting the unmethylated cytosine to uracil. Because methylated cytosines are protected from deamination, they do not undergo this conversion and the primers can be designed to distinguish between the sequences of the treated and untreated nucleic acids in a predictable, methylation-dependent way.

Any of the exemplary assay formats described herein can be adapted or optimized for use in automated and semi-automated systems (including those in which there is a solid phase comprising a microparticle), for example as described, e.g., in U.S. Pat. Nos. 5,089,424 and 5,006,309, and in connection with any of the commercially available detection platforms known in the art.

In certain embodiments, the methods and/or assays of the present invention are directed to the detection of all or a part of the attractor molecular signature wherein such detection can take the form of either a binary, detected/not-detected, result. In certain embodiments, the methods, assays, and/or kits of the present invention are directed to the detection of all or a part of the attractor molecular signature wherein such detection can take the form of a multi-factorial result. For example, but not by way of limitation, such multi-factorial results can take the form of a score based on one, two, three, or more factors. Such factors can include, but are not limited to: (1) detection of a change in expression of an attractor molecular signature gene product, state of methylation, and/or presence of microRNA; (2) the number of attractor molecular signature gene products, states of methylation, and/or presence of microRNAs in a sample exhibiting an altered level; and (3) the extent of such change in attractor molecular signature gene products, states of methylation, and/or presence of microRNAs.

### 4.3.3. Kits Comprising Attractor Molecular Signatures for Diagnosis and/or Treatment

In certain embodiments, compositions useful in the detection and/or assaying of one or more attractor molecular signature of the present invention can be packaged into kits. In certain embodiments, the kit will include compositions for detecing one, two, three, four, five, six, seven, eight, or all nine of the following features: FGD3-SUSD3, CIN, MES*, LYM, END, LYM*, CD68, DNAJB9, CXCL12.

In certain embodiments, a kit may comprise a pair of oligonucleotide primers, suitable for polymerase chain reaction, for each gene and/or gene product to be measured. Such primers may be designed based on the sequences for the genes associated with said attractor molecular signature(s).

In certain embodiments the kit will include a measurement means, such as, but not limited to a microarray. In certain non-limiting embodiments, where the measurement means in the kit employs a microarray, the set of markers associated with the attractor metagene may constitute at least 10 percent or at least 20 percent or at least 30 percent or at least 40 percent or at least 50 percent or at least 60 percent or at least 70 percent or at least 80 percent of the species of markers represented on the chip.

Any of the foregoing kits, in this or the preceding sections, may further optionally comprise one or more controls such as a healthy control, or any other appropriate control to allow for diagnosis. In non-limiting examples, such controls may be plasma samples or may be combinations of genes and/or gene products prepared to resemble such natural plasma samples.

### 5. EXAMPLES

### 5.1 Example 1

### 5.1.1. Pan-Cancer Molecular Signatures

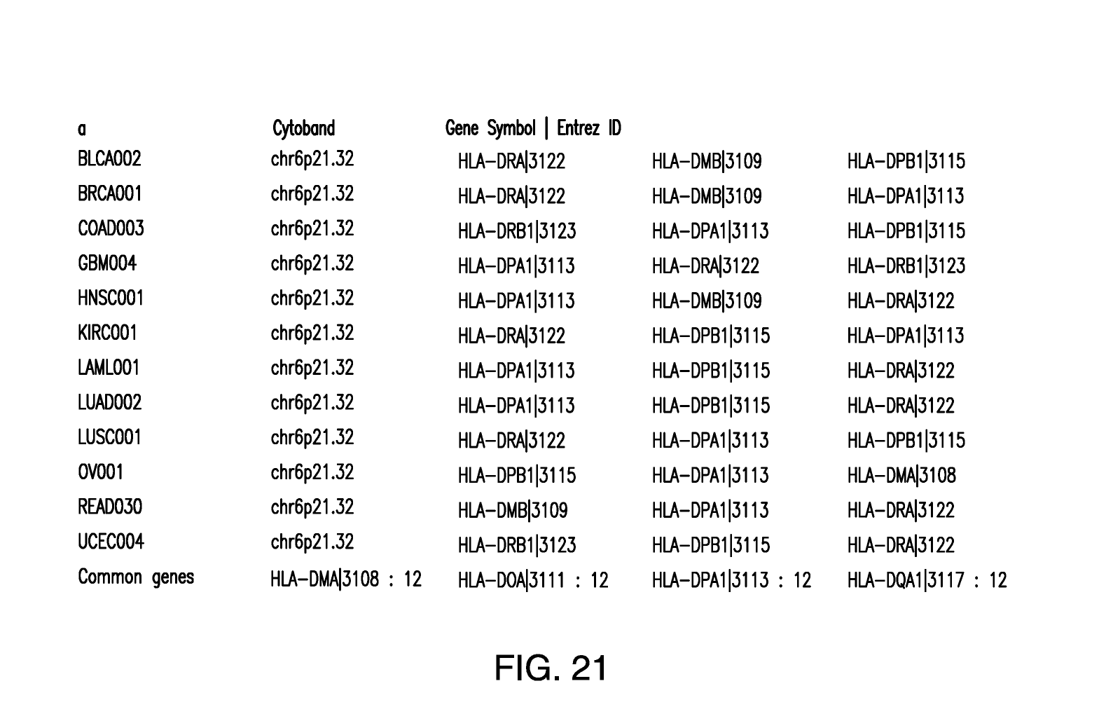

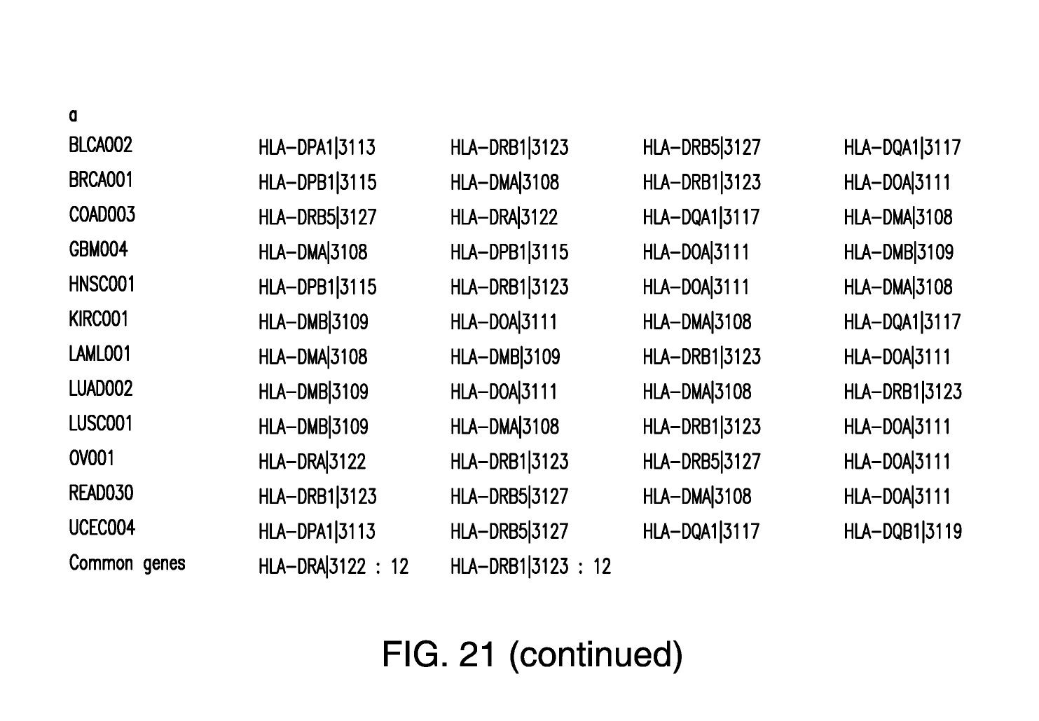

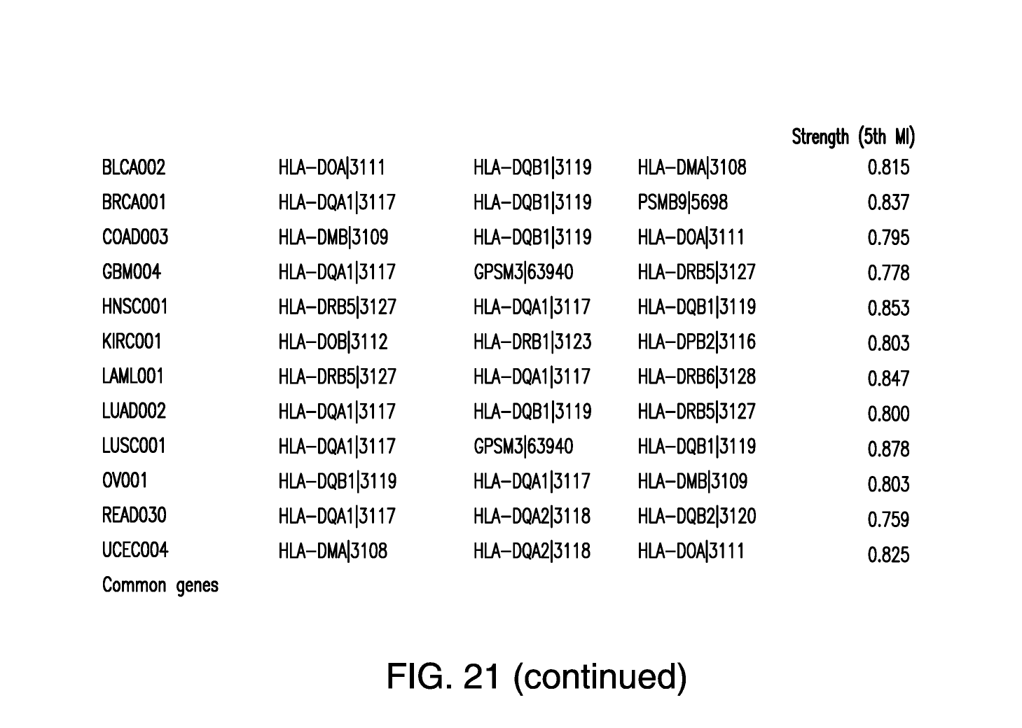

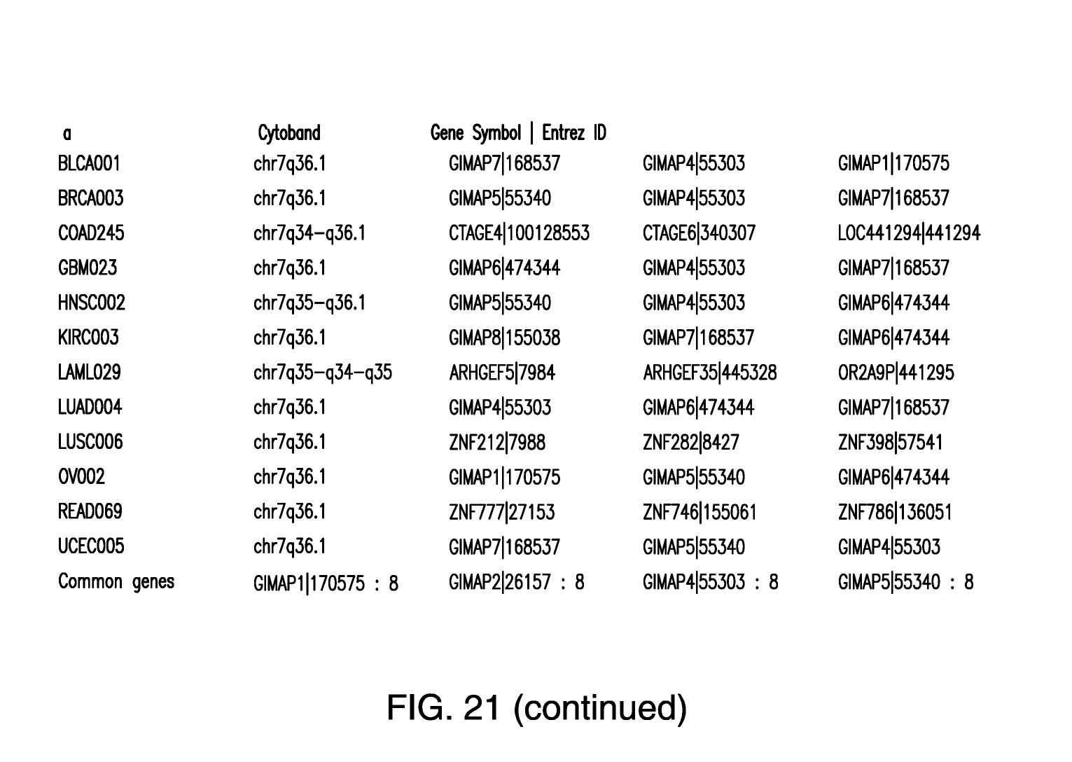

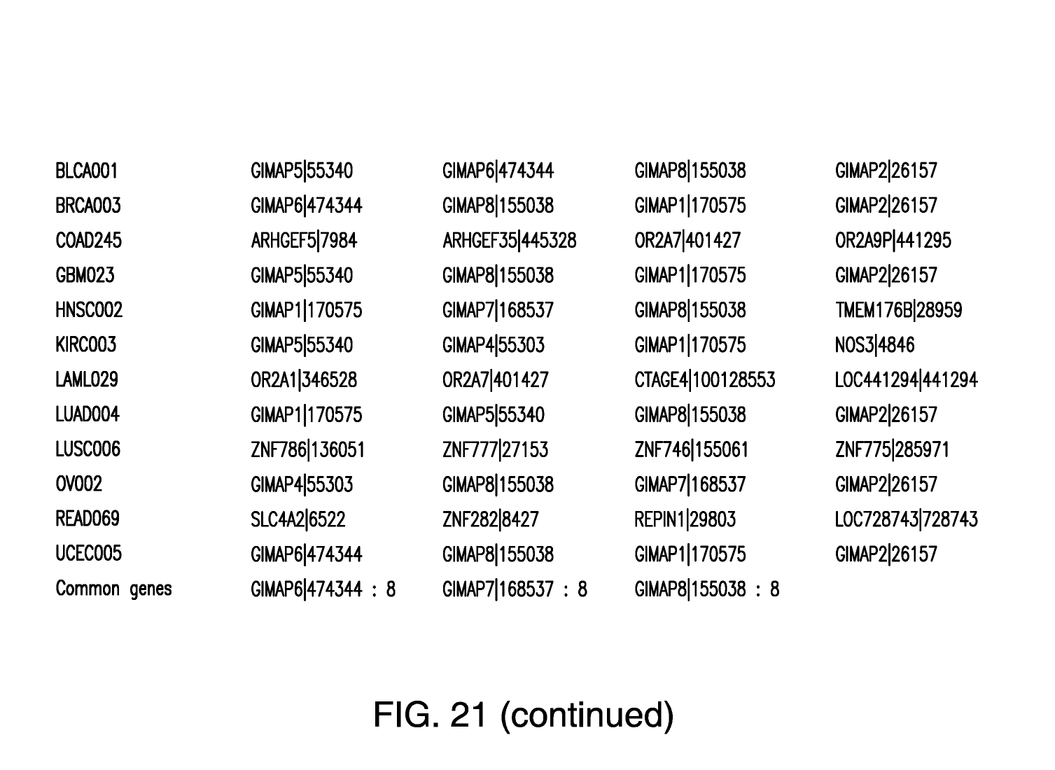

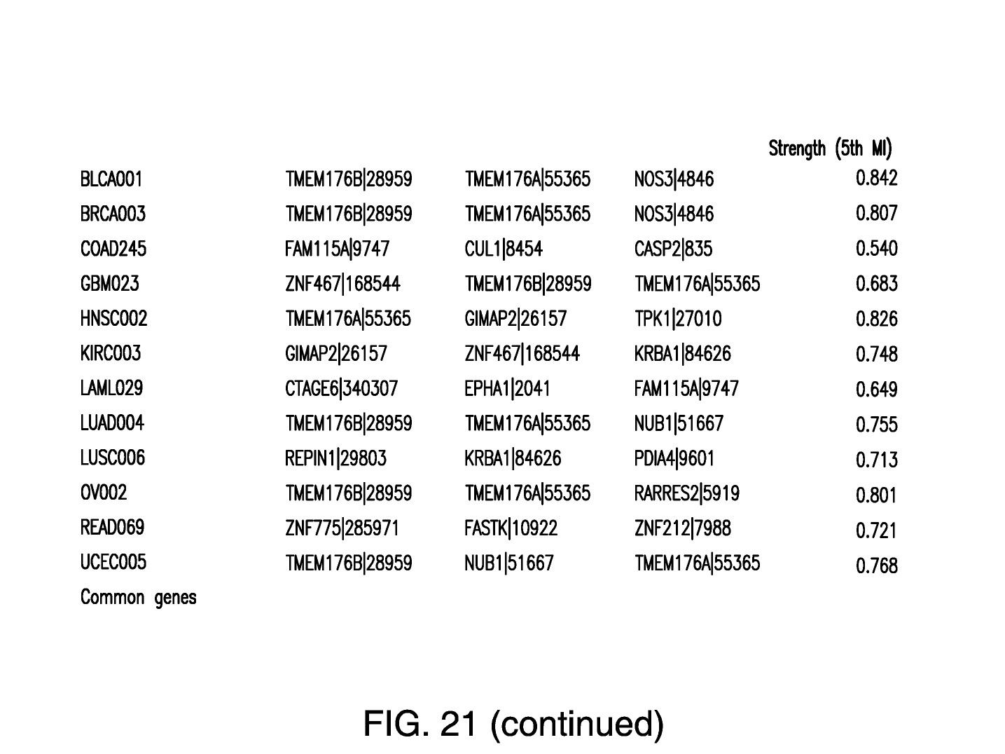

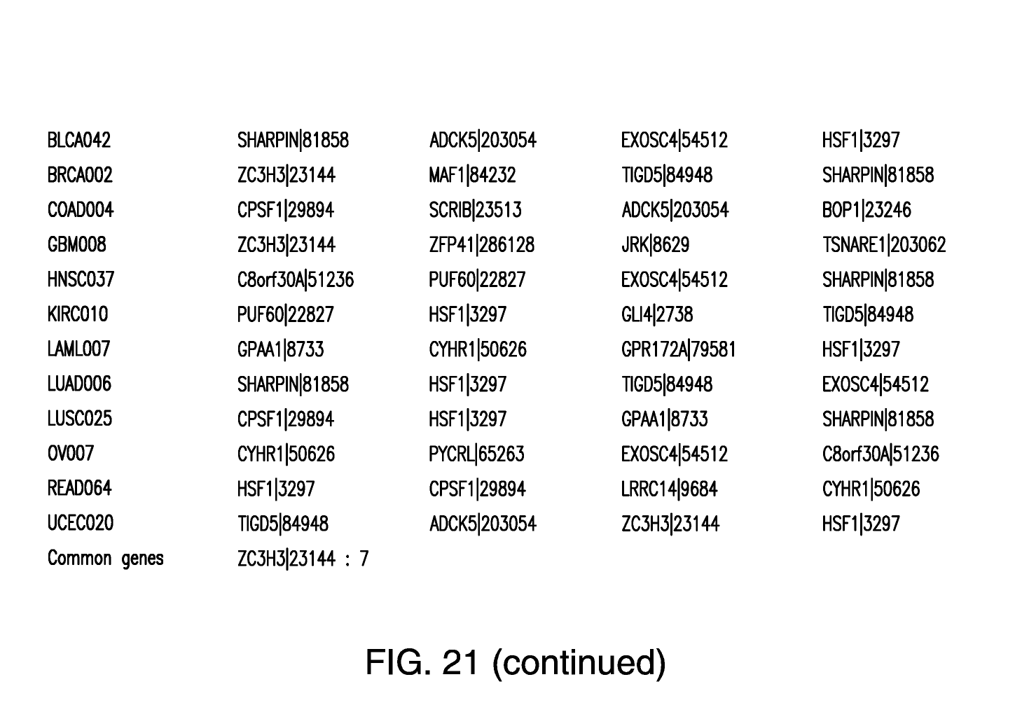

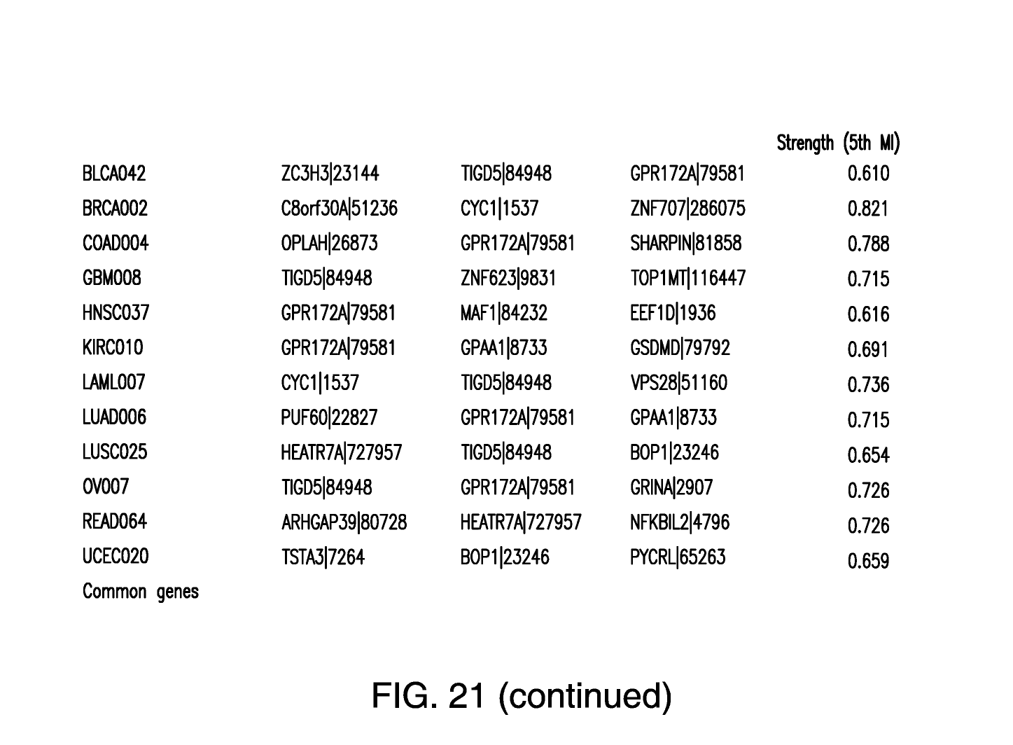

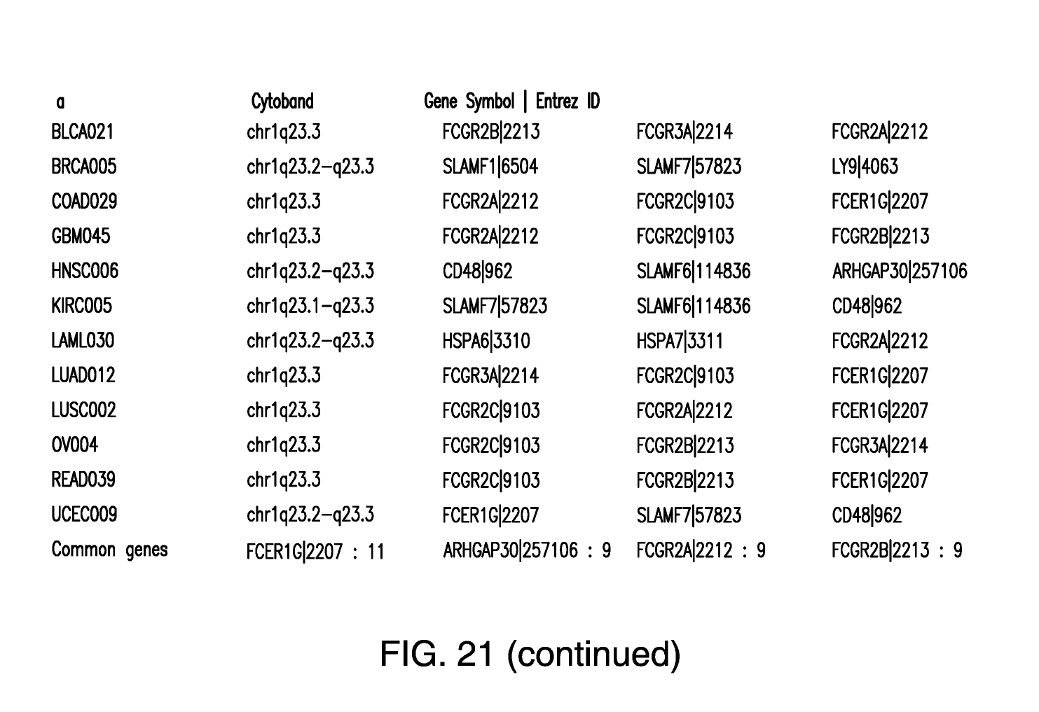

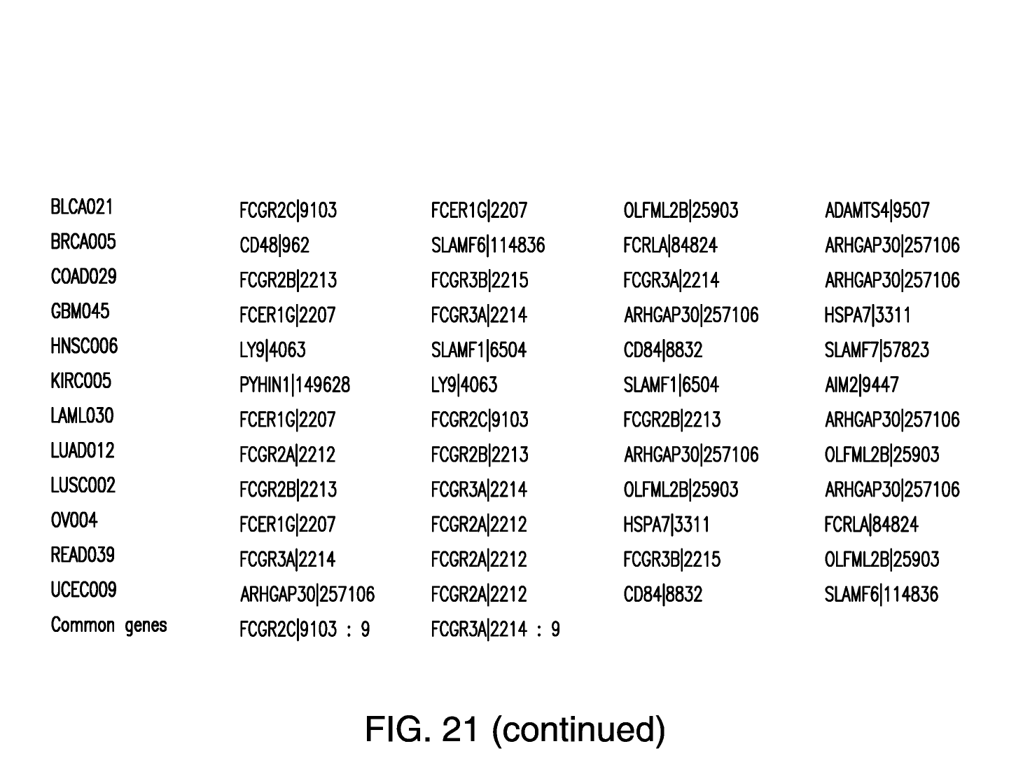

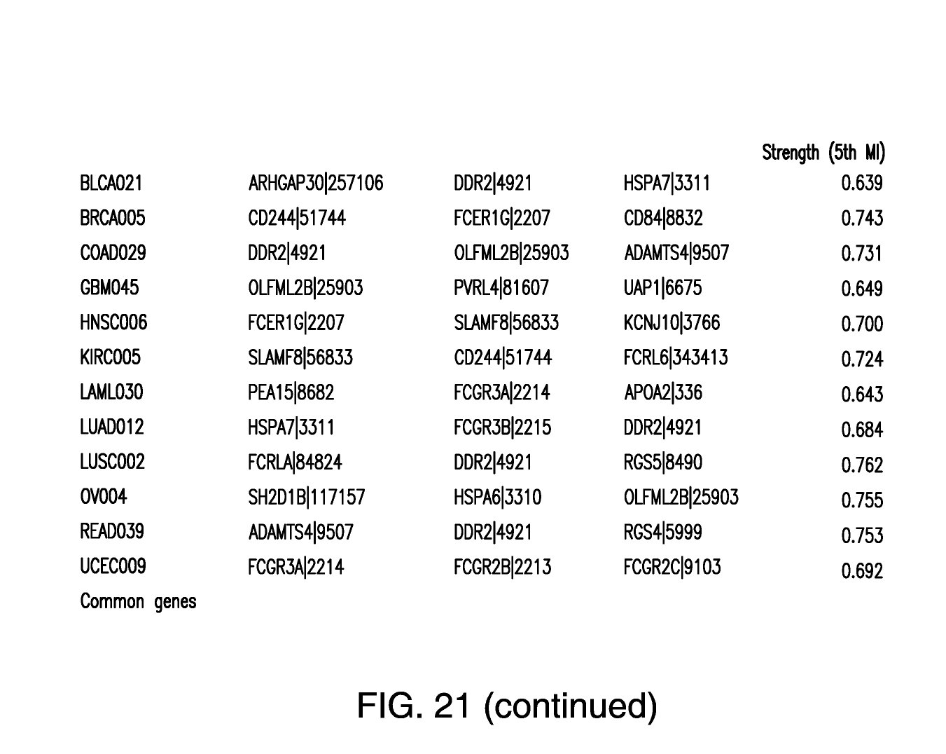

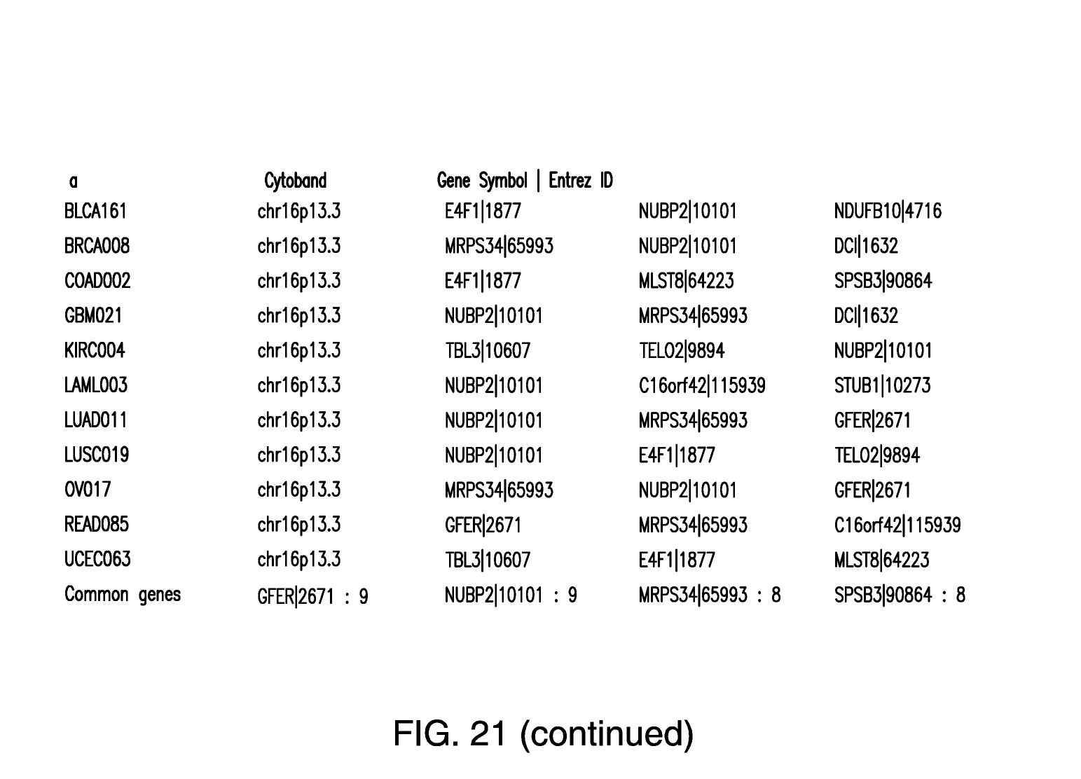

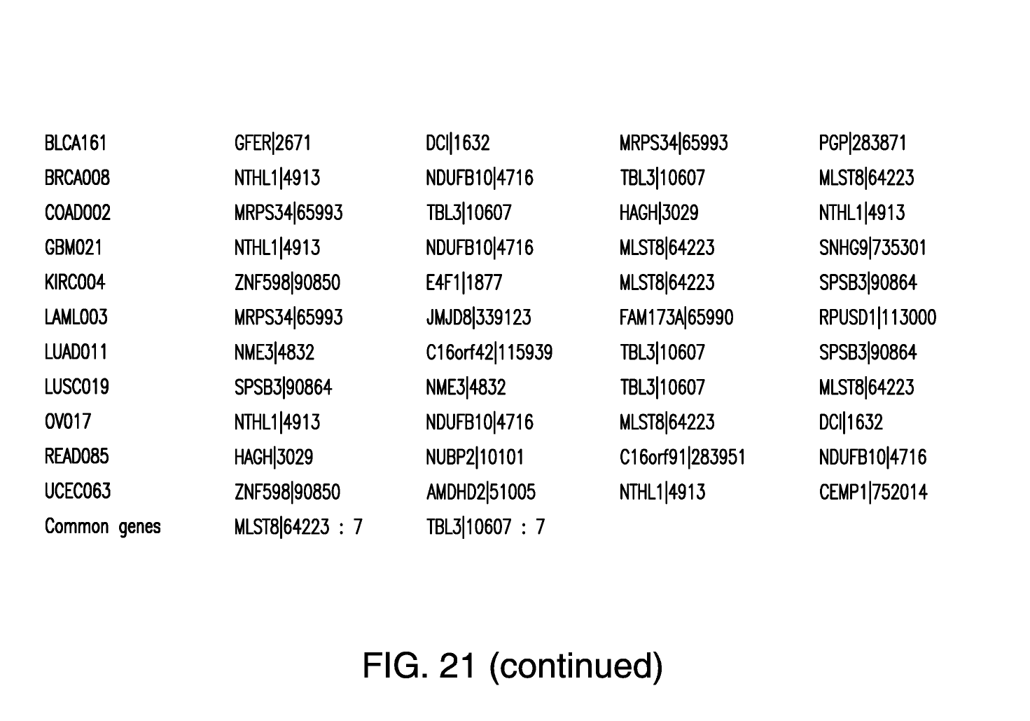

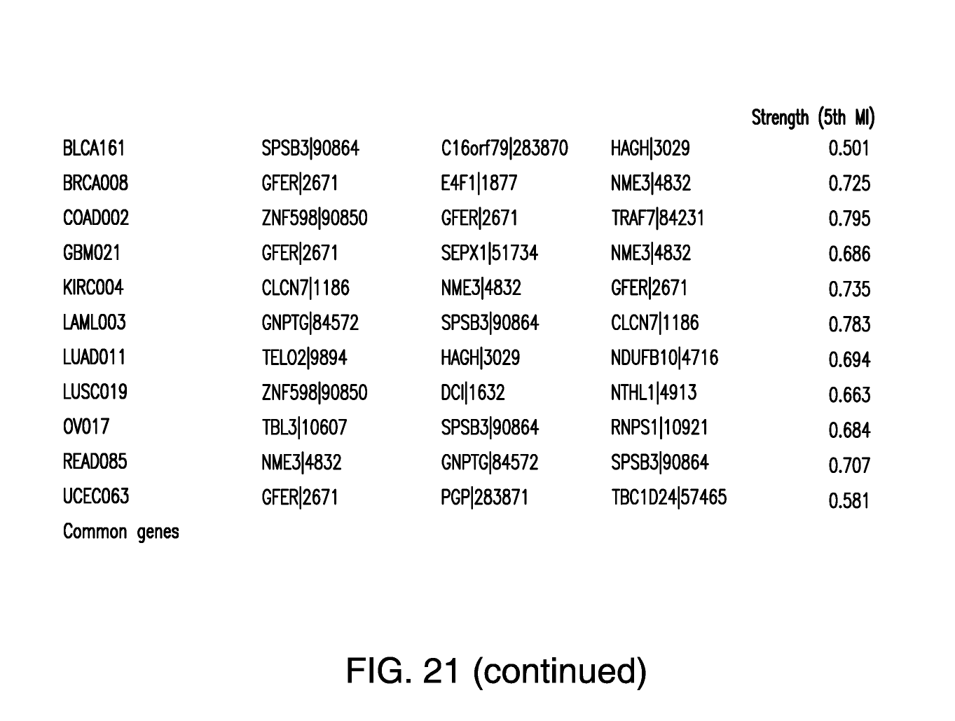

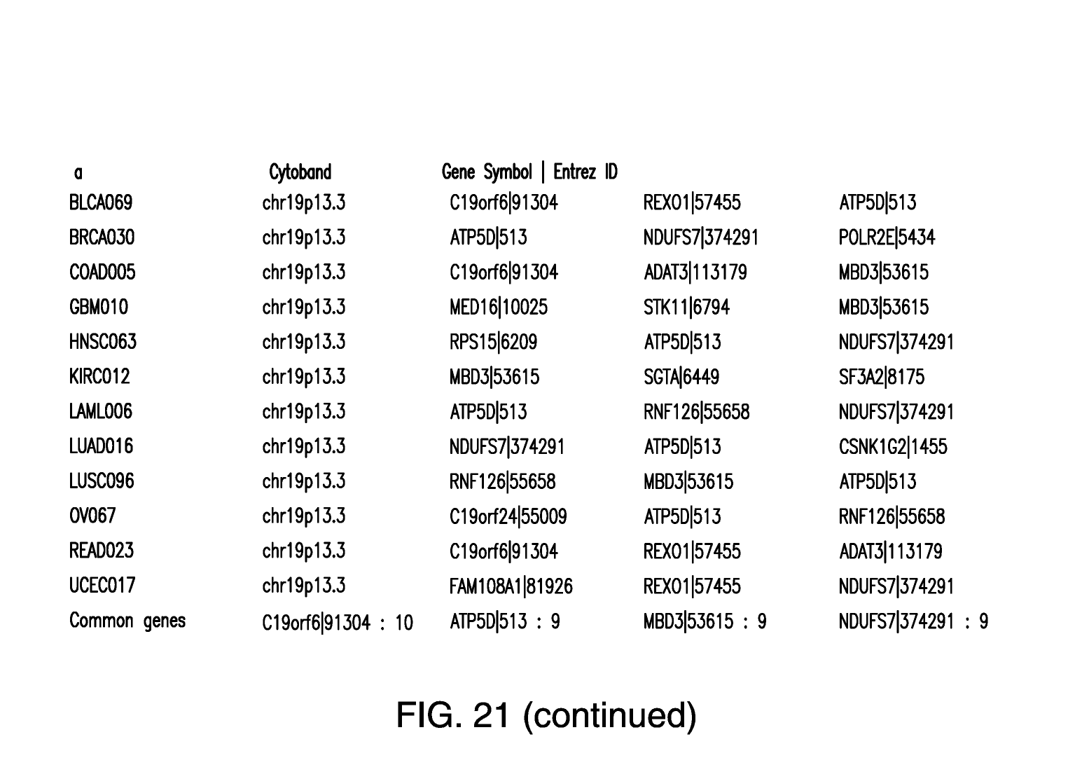

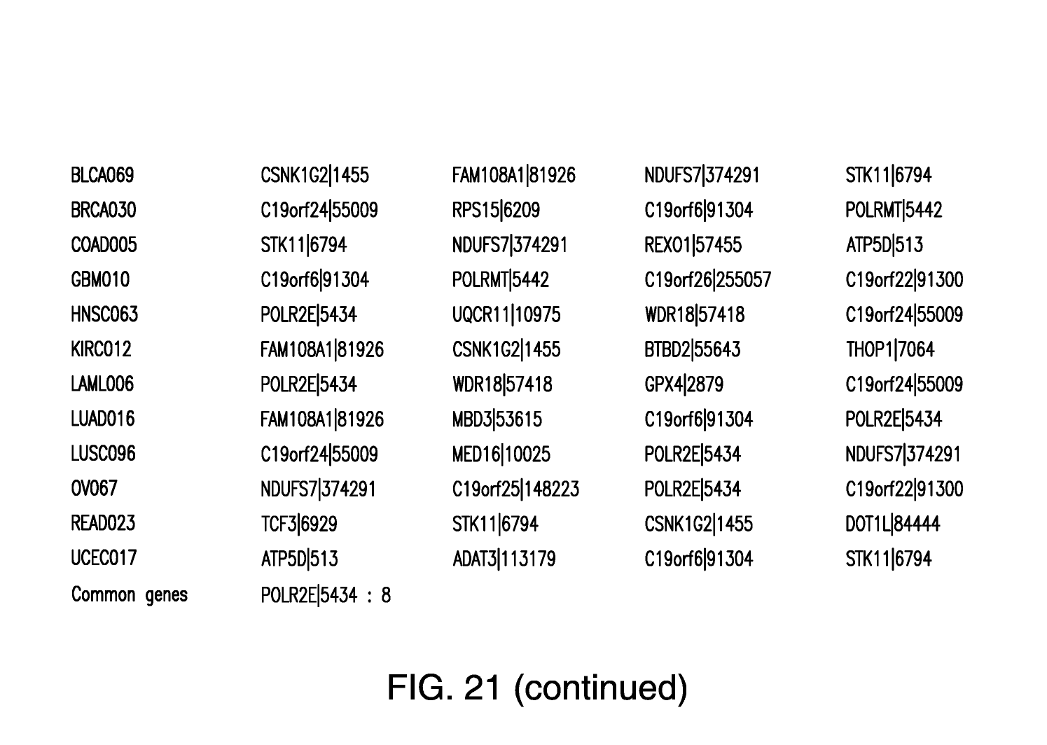

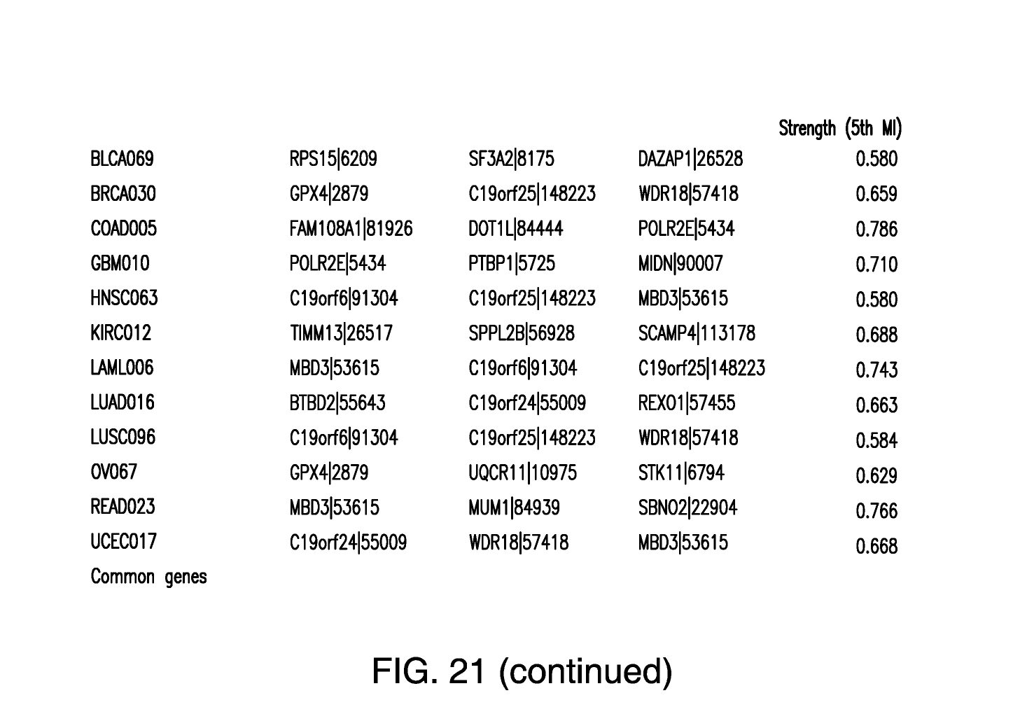

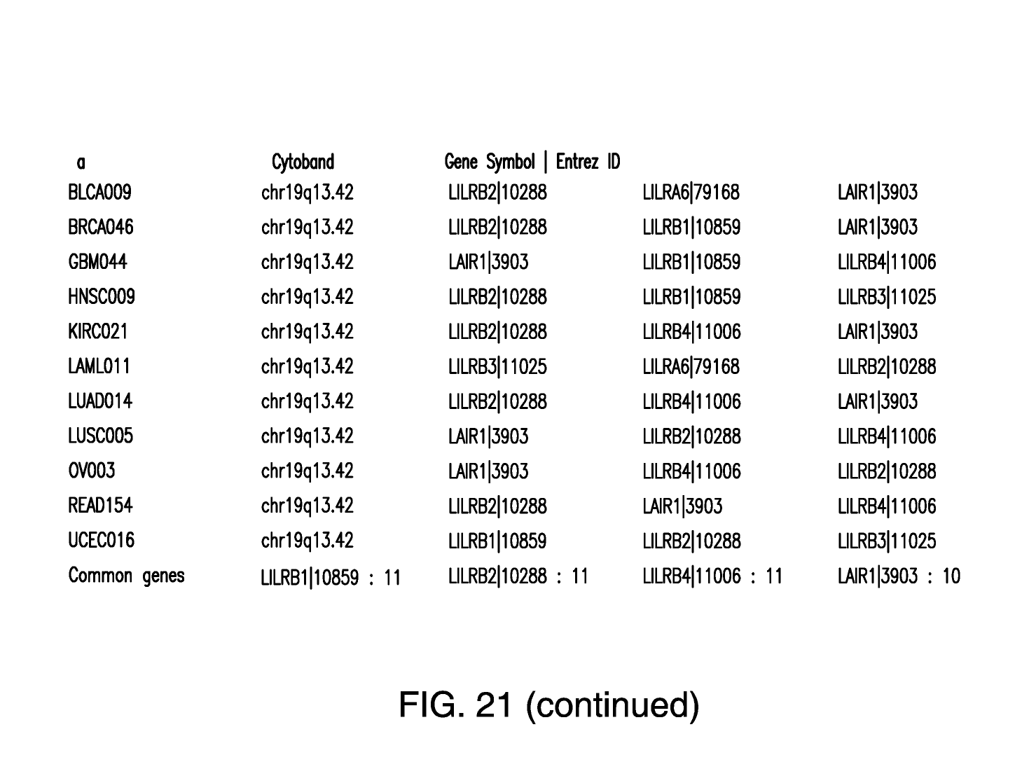

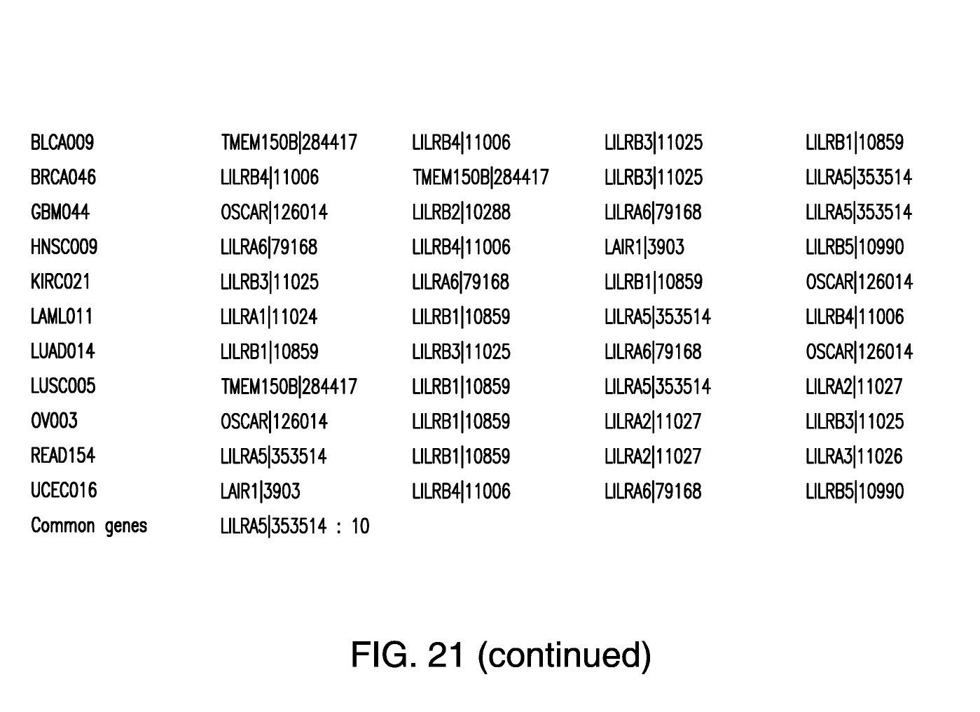

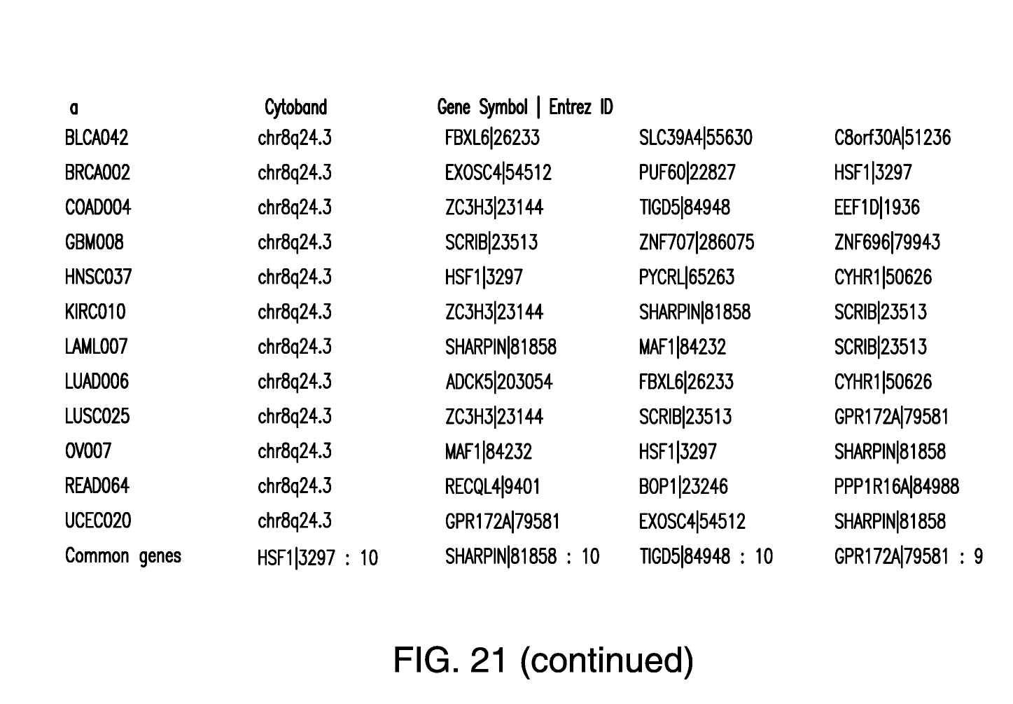

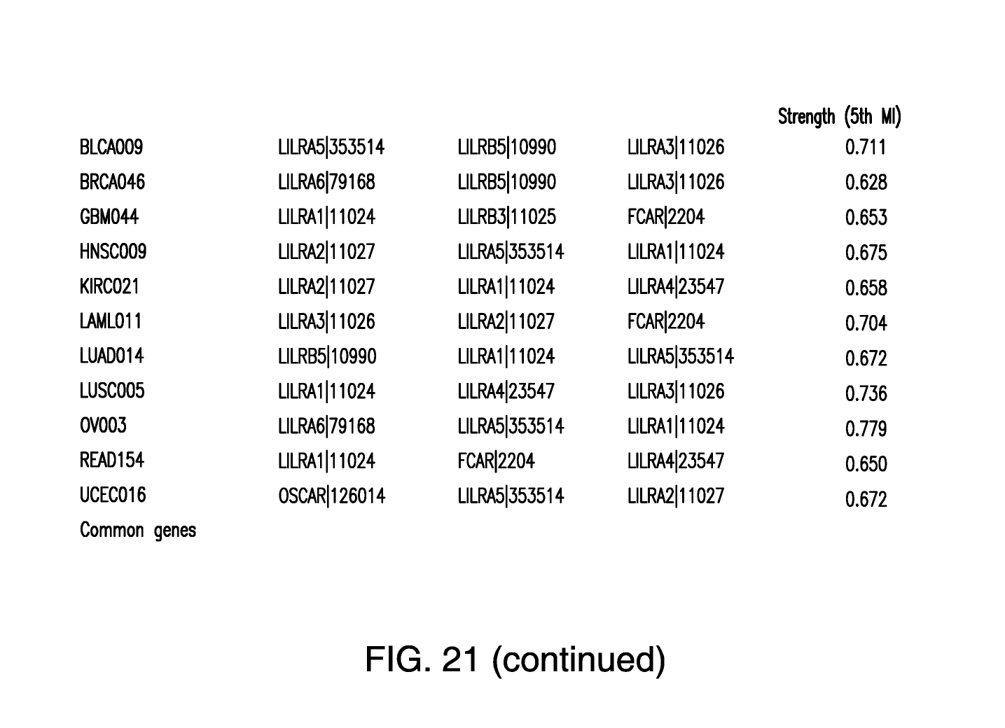

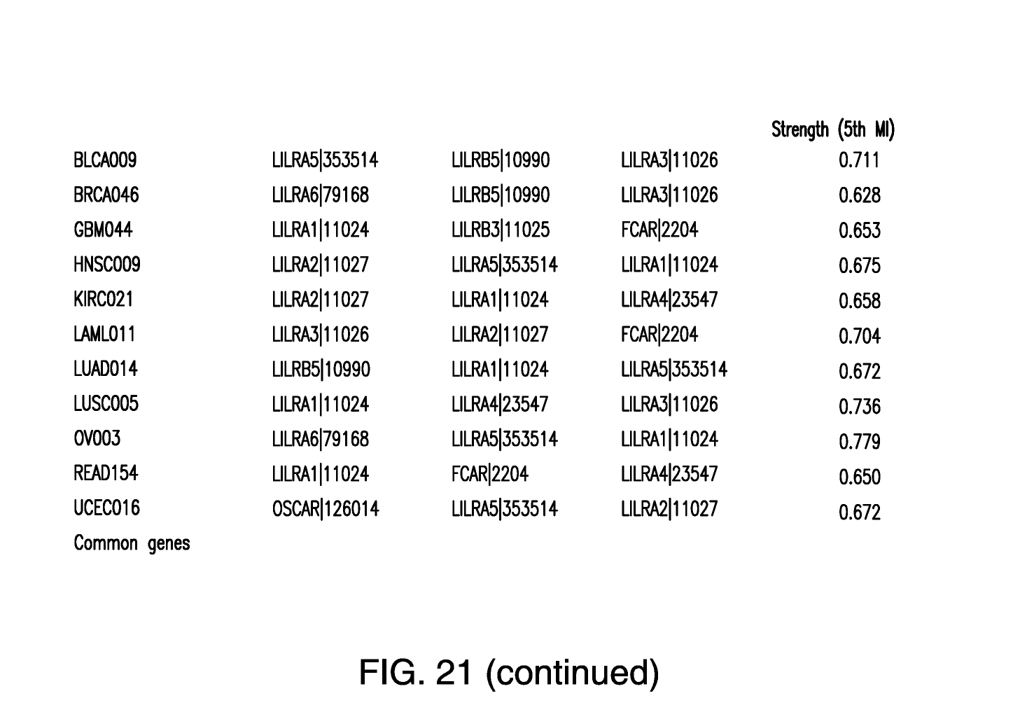

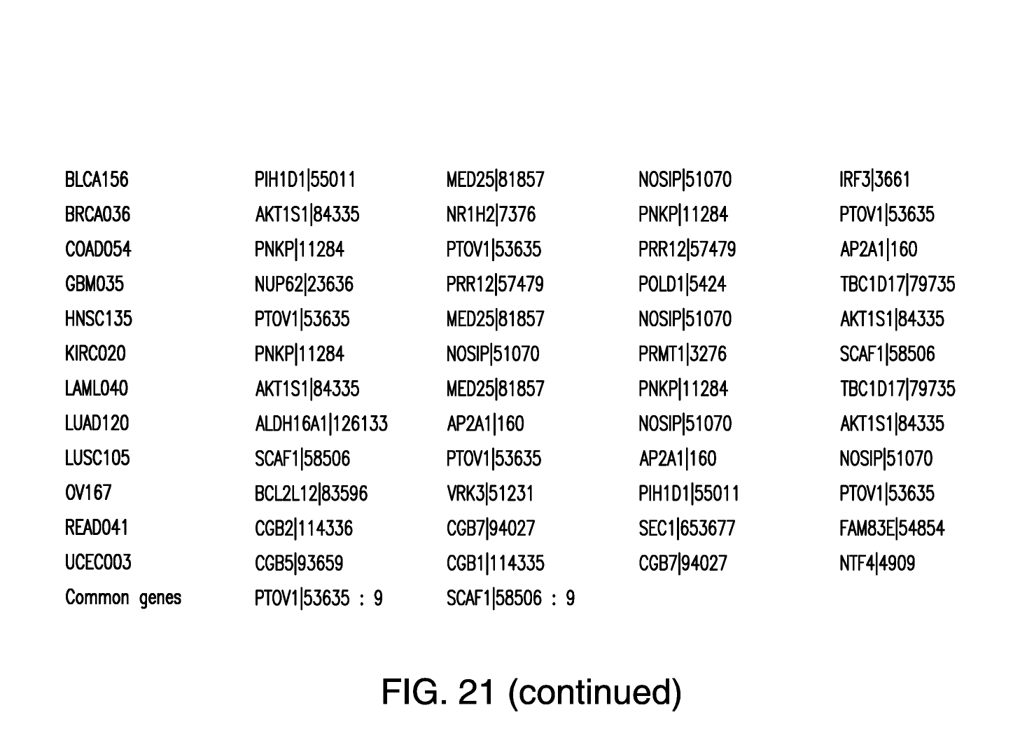

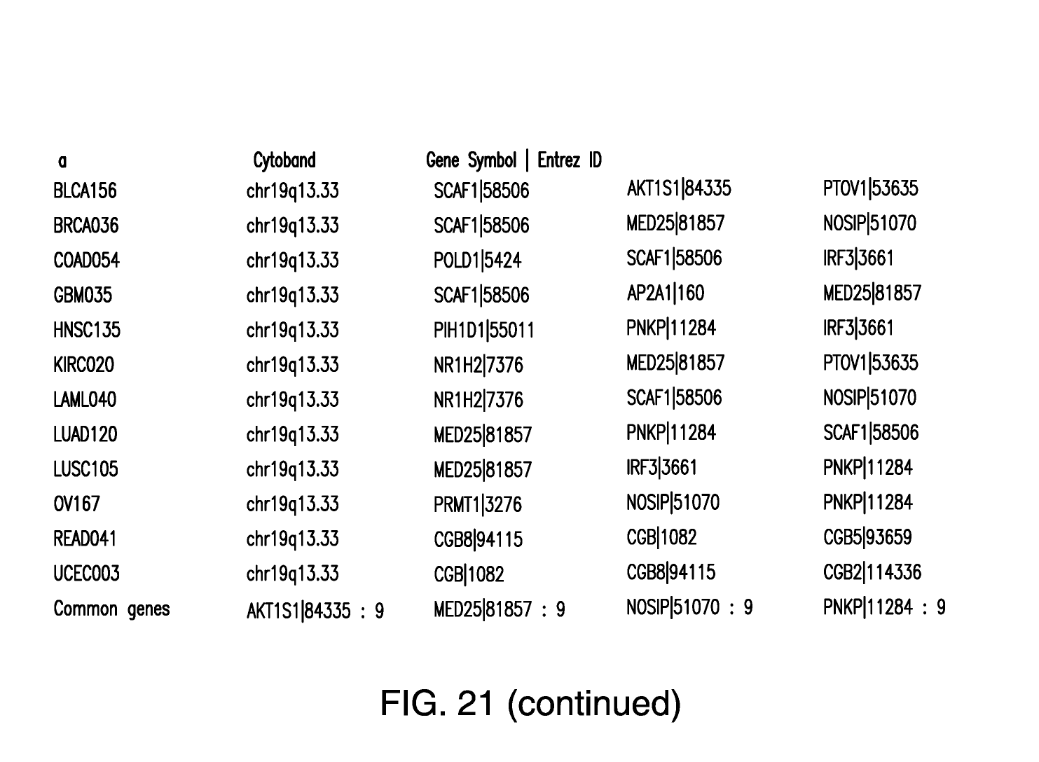

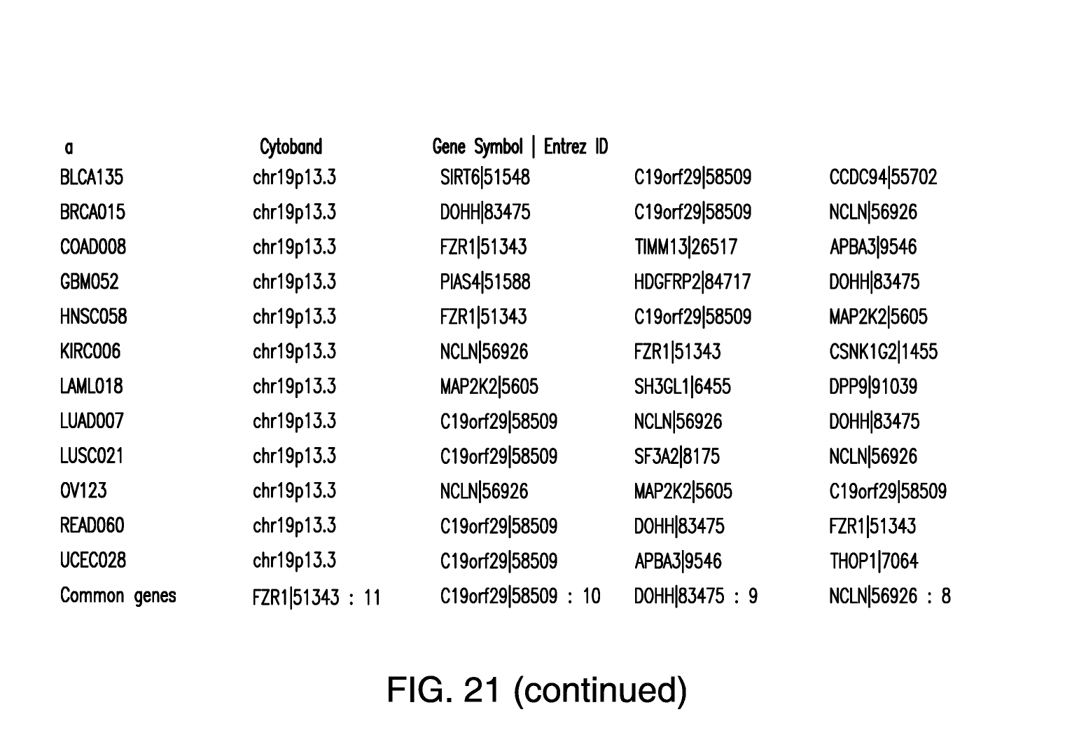

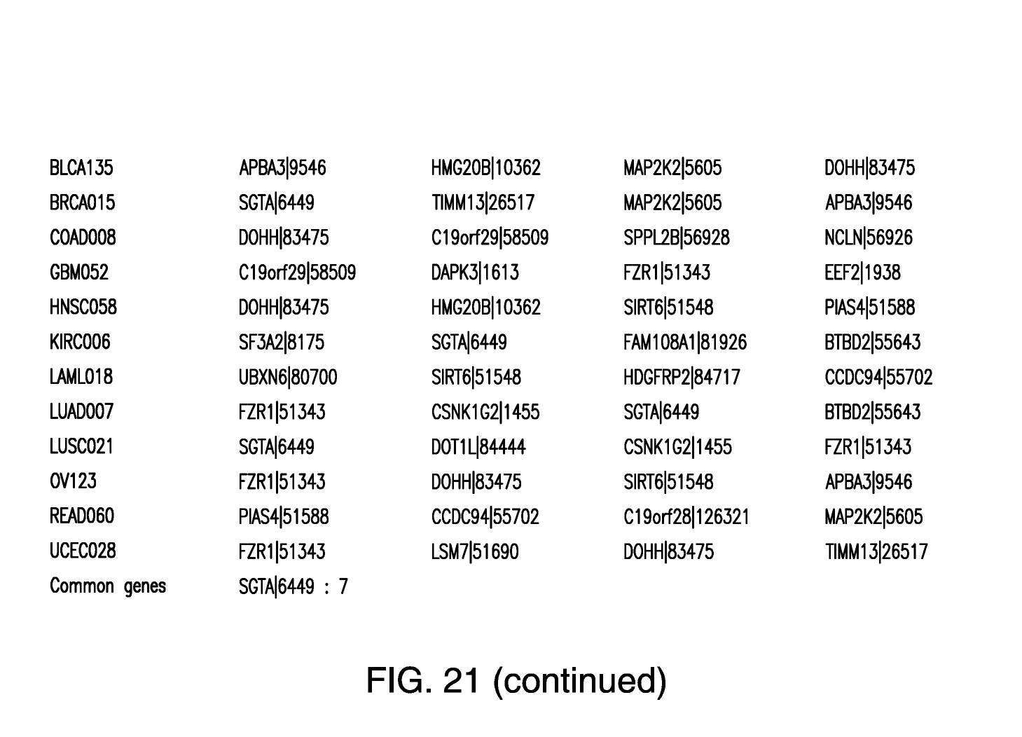

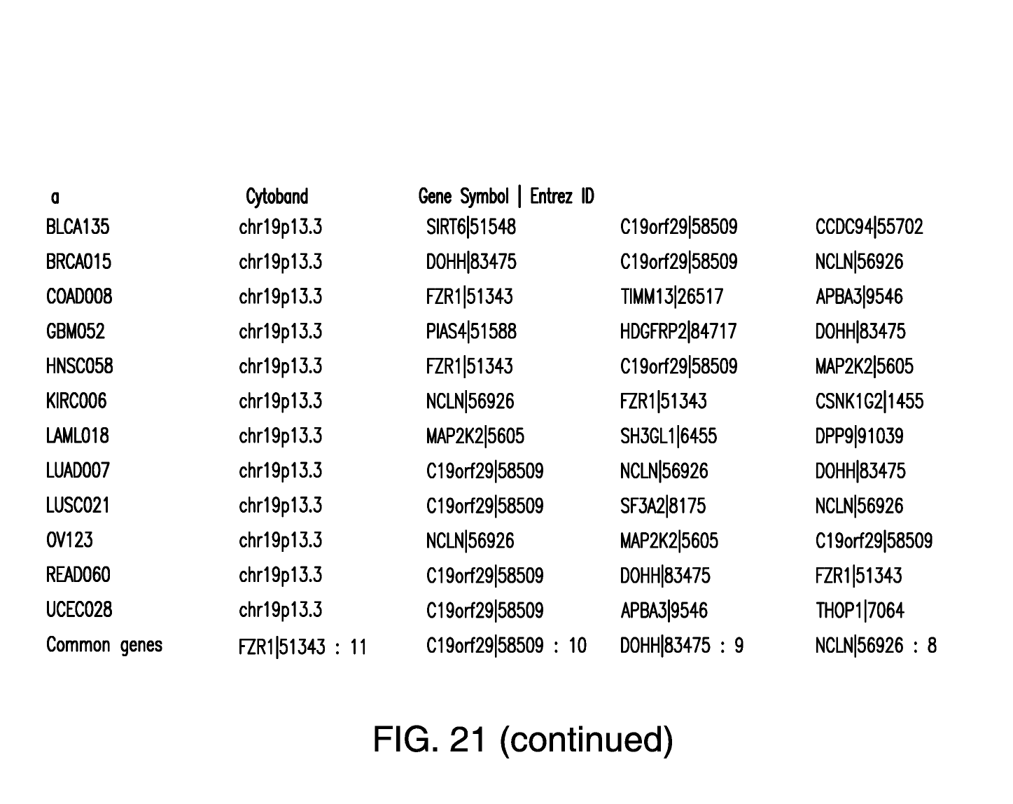

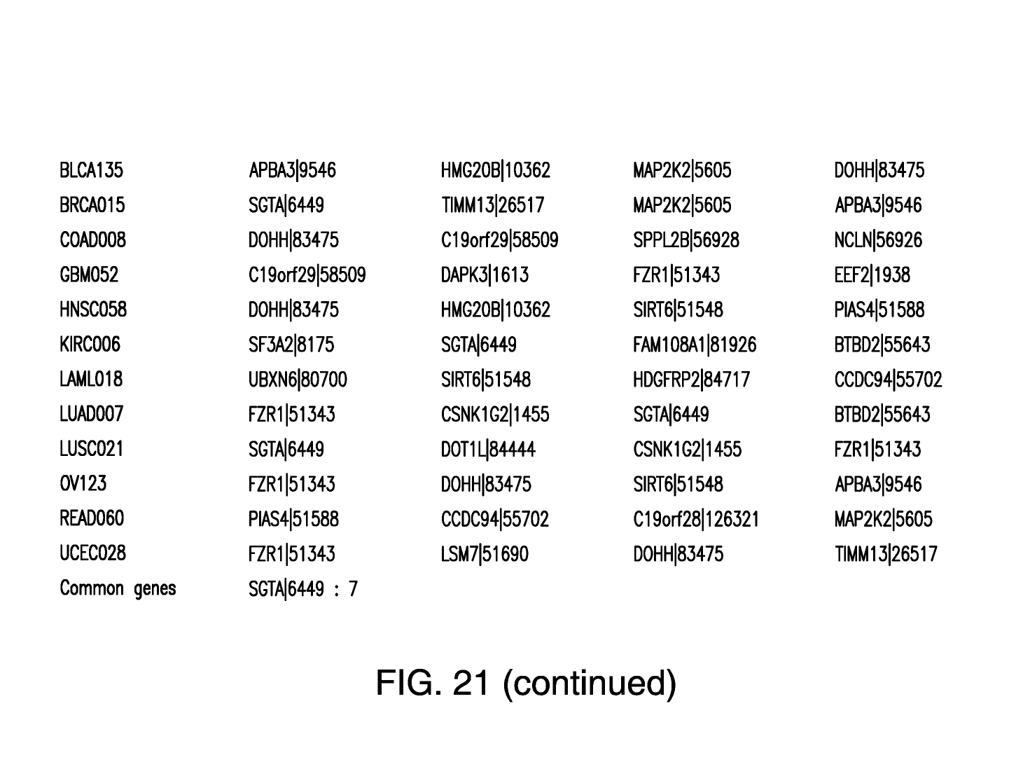

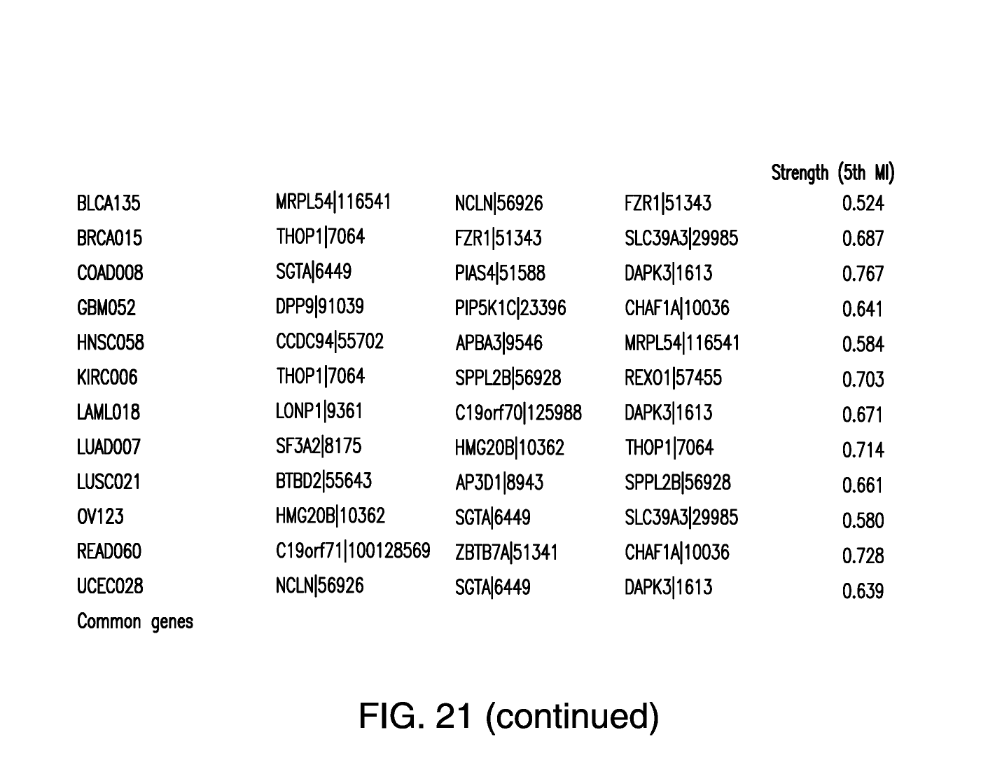

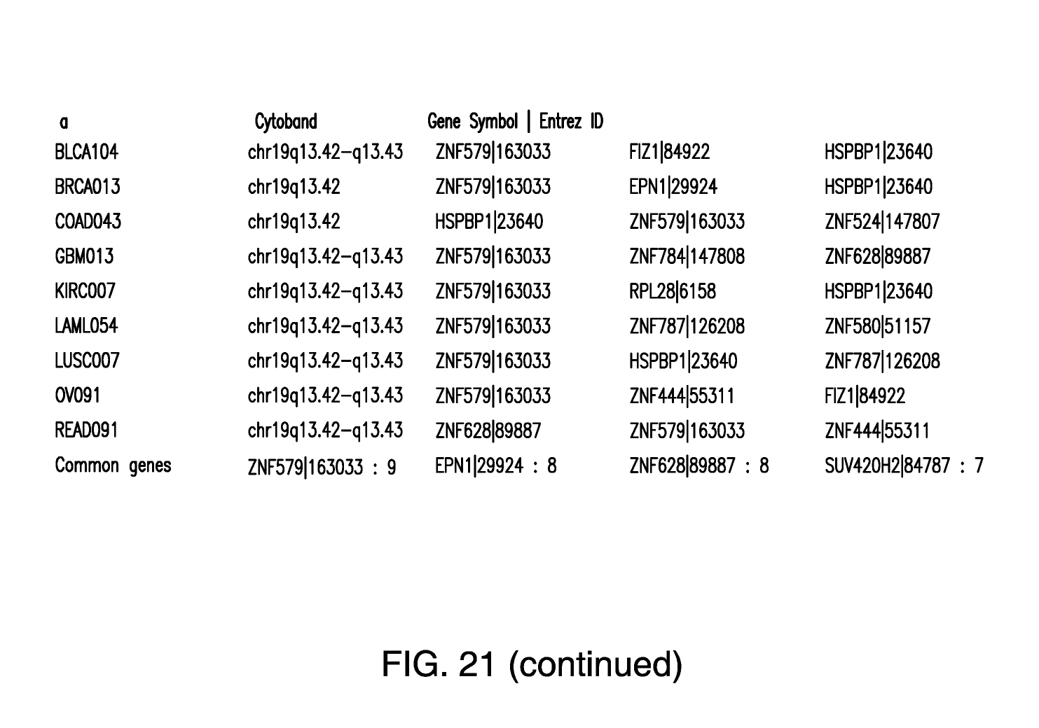

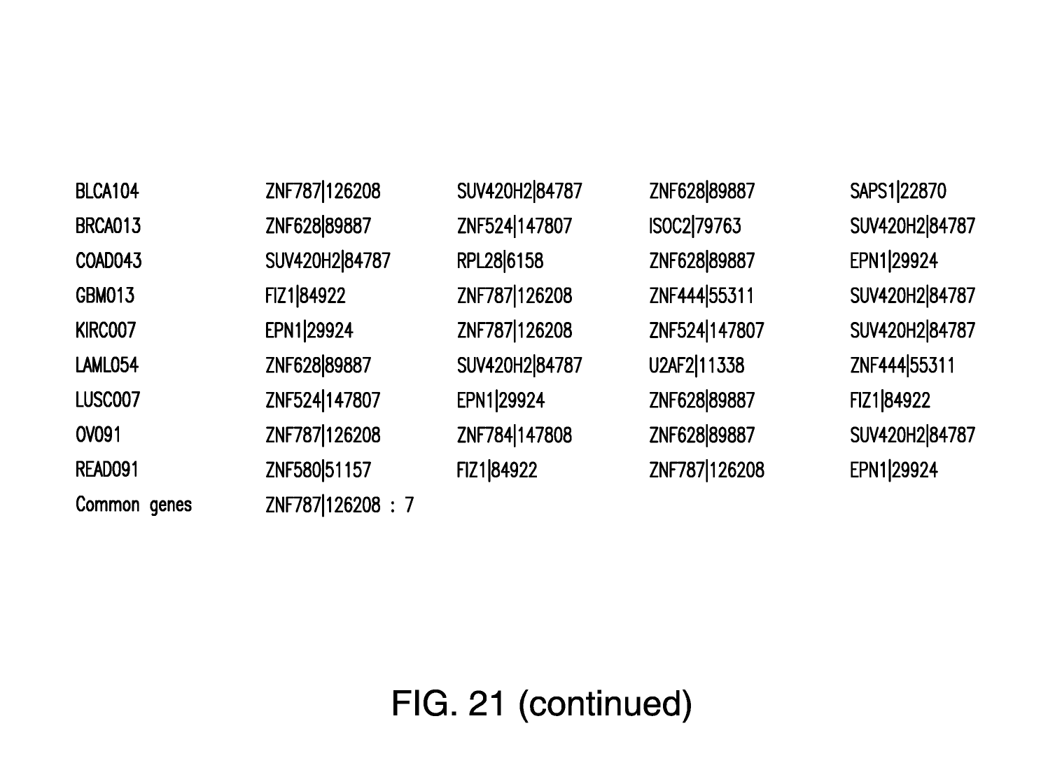

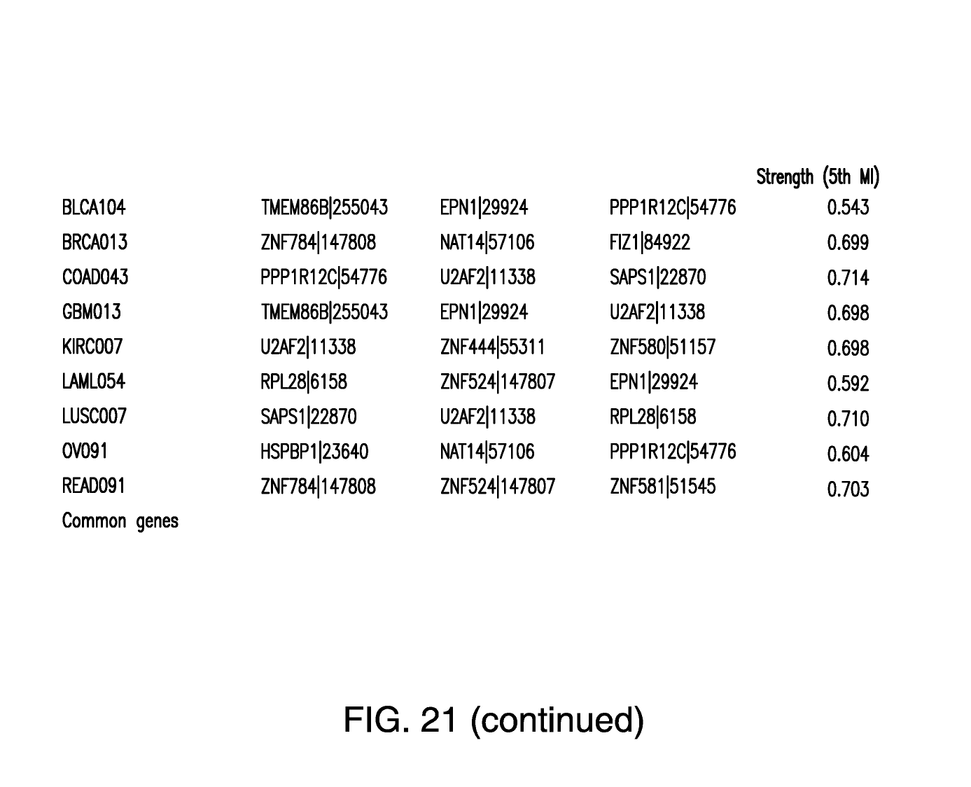

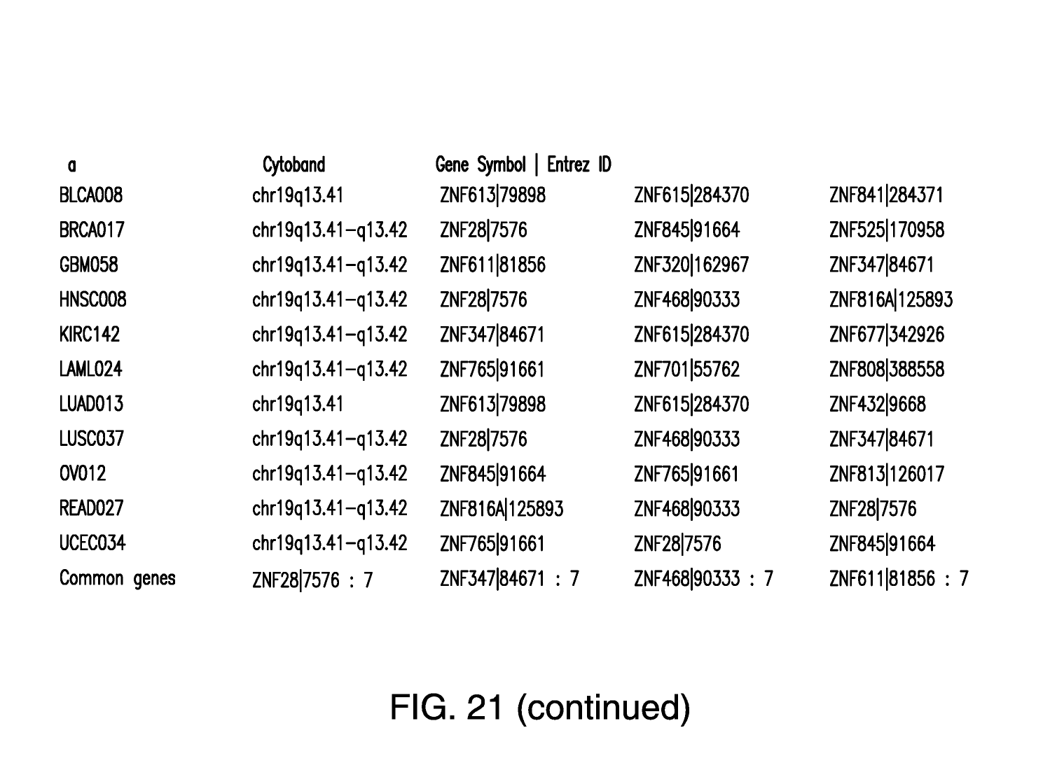

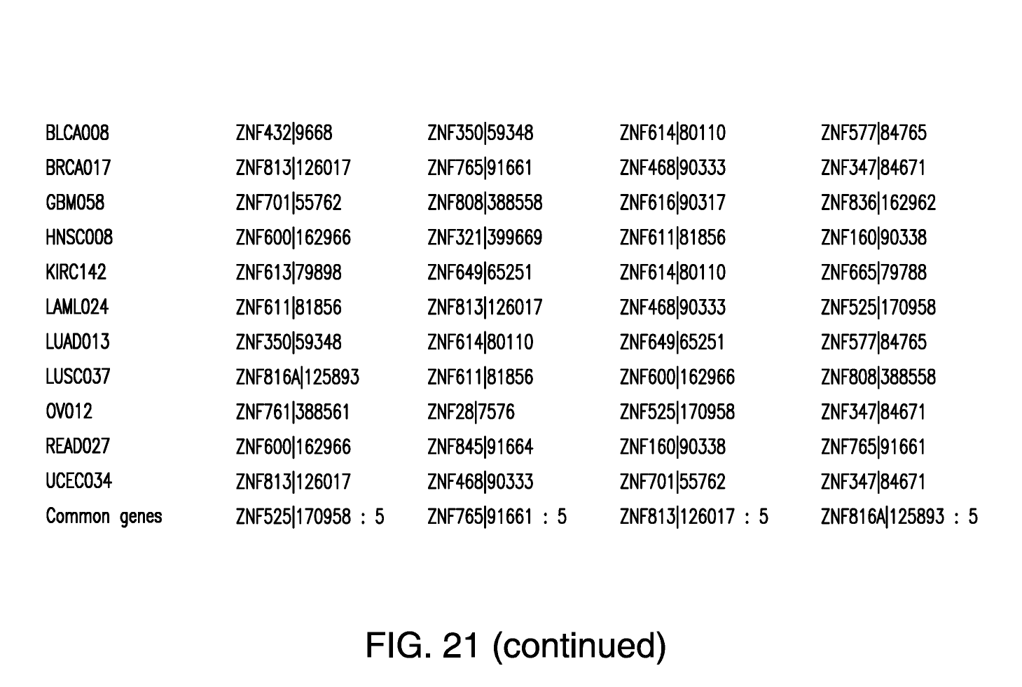

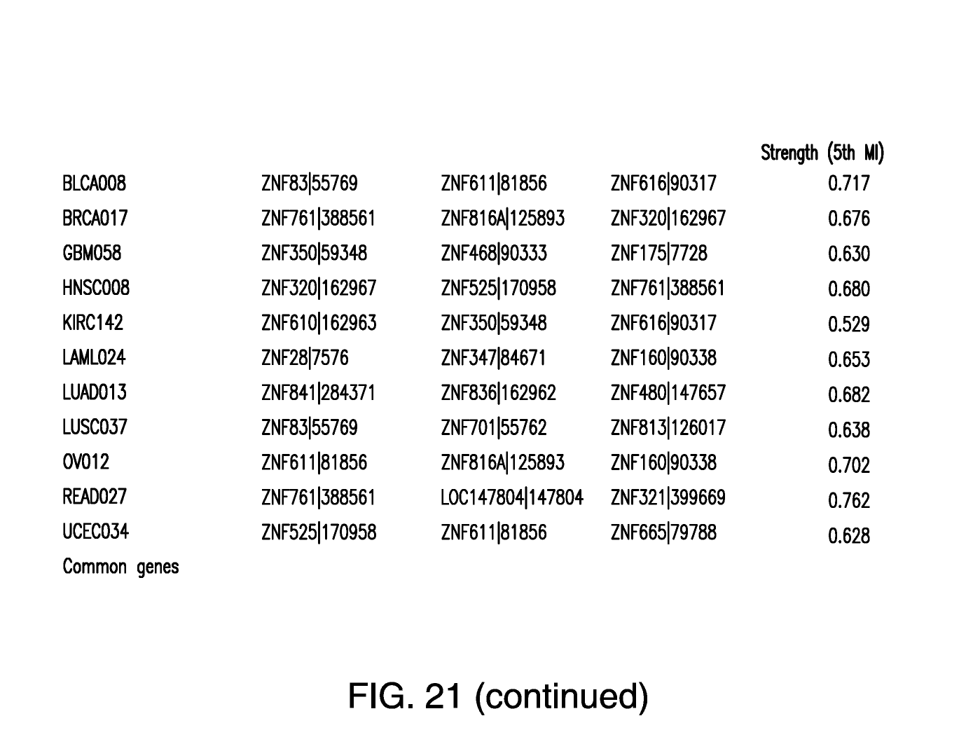

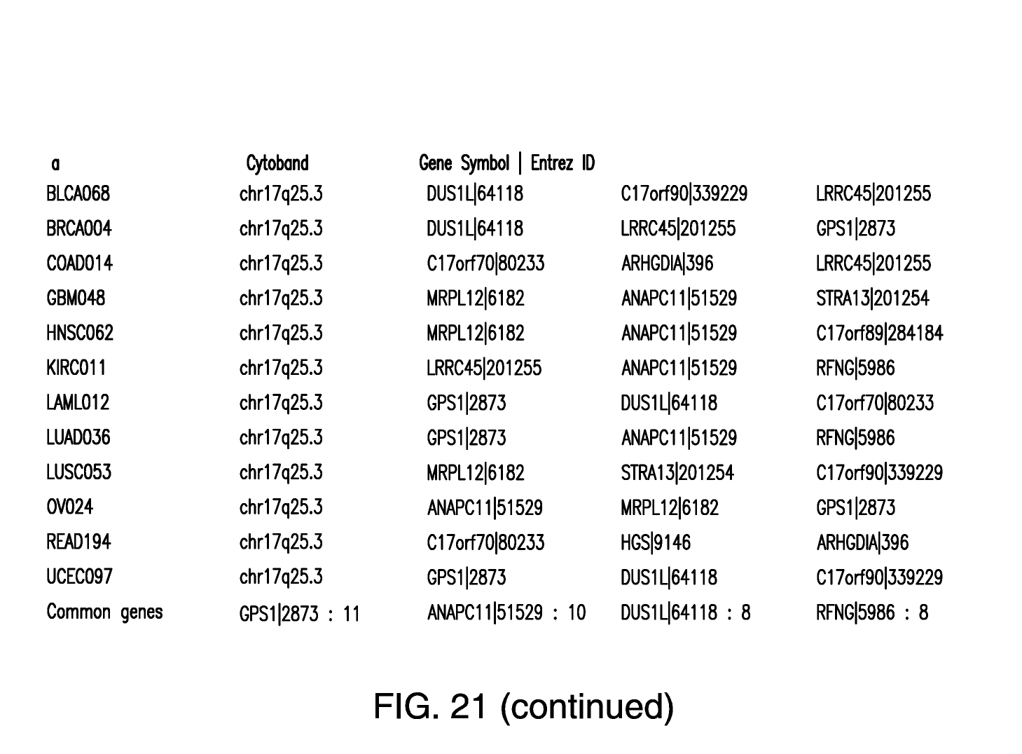

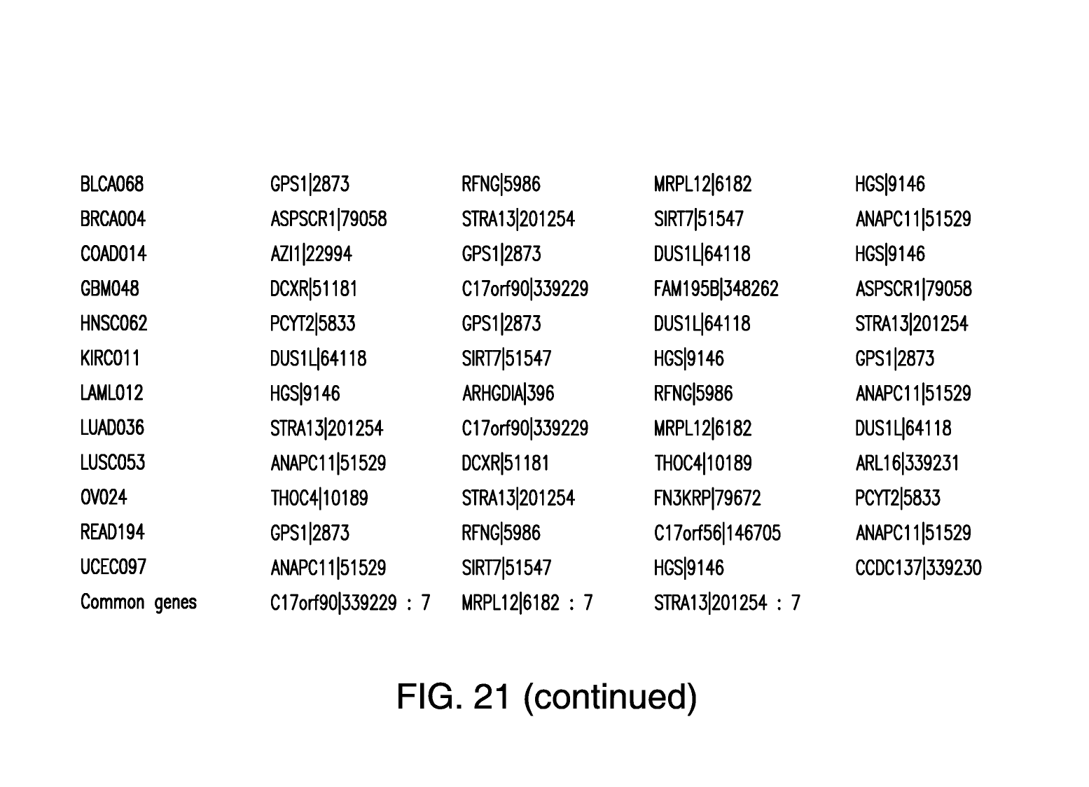

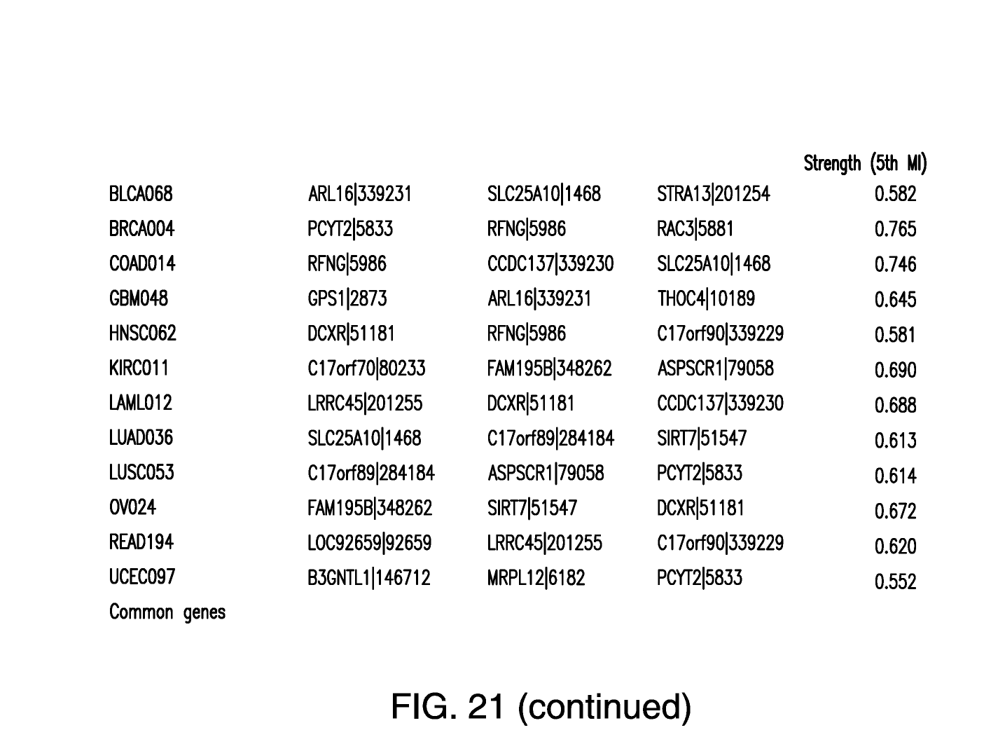

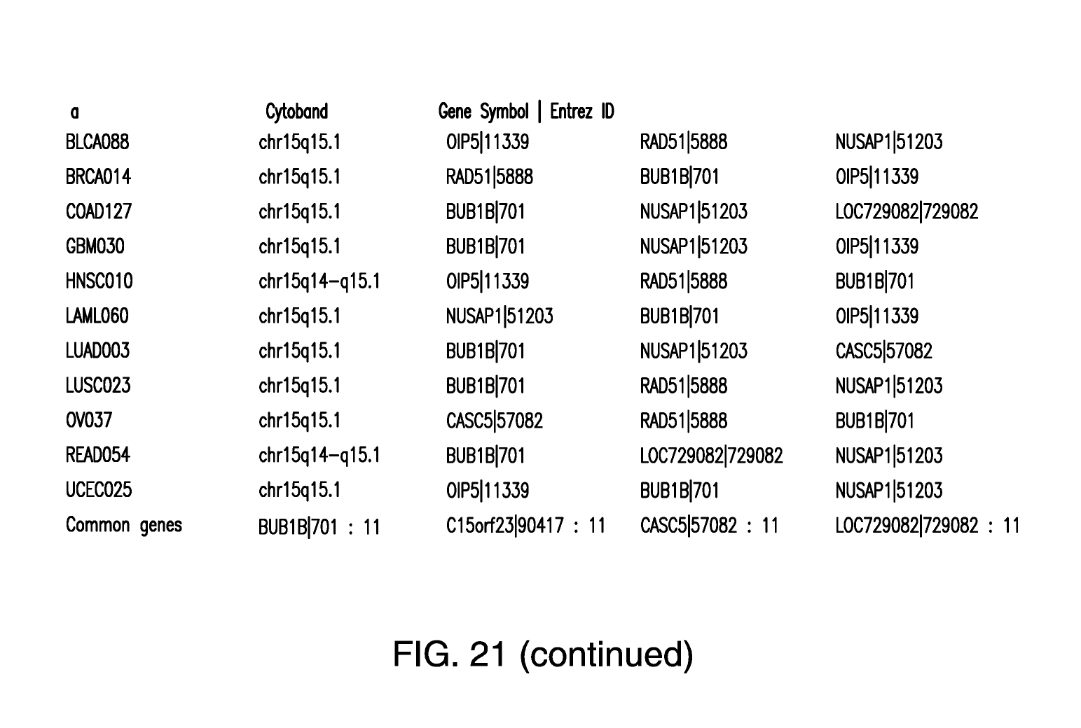

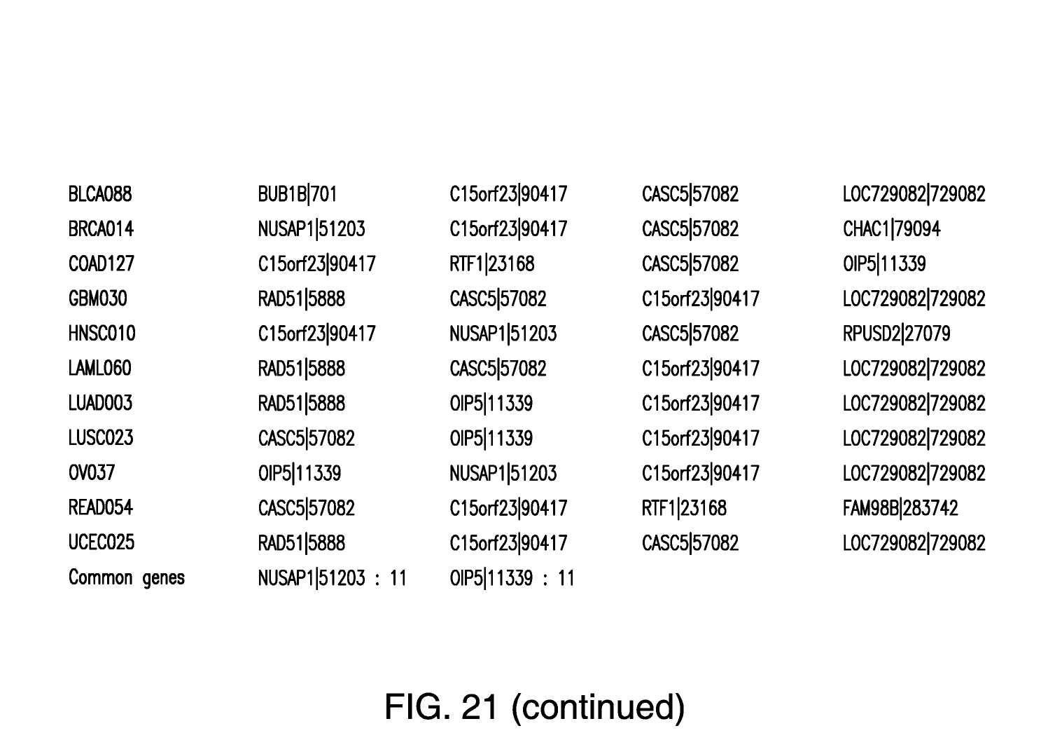

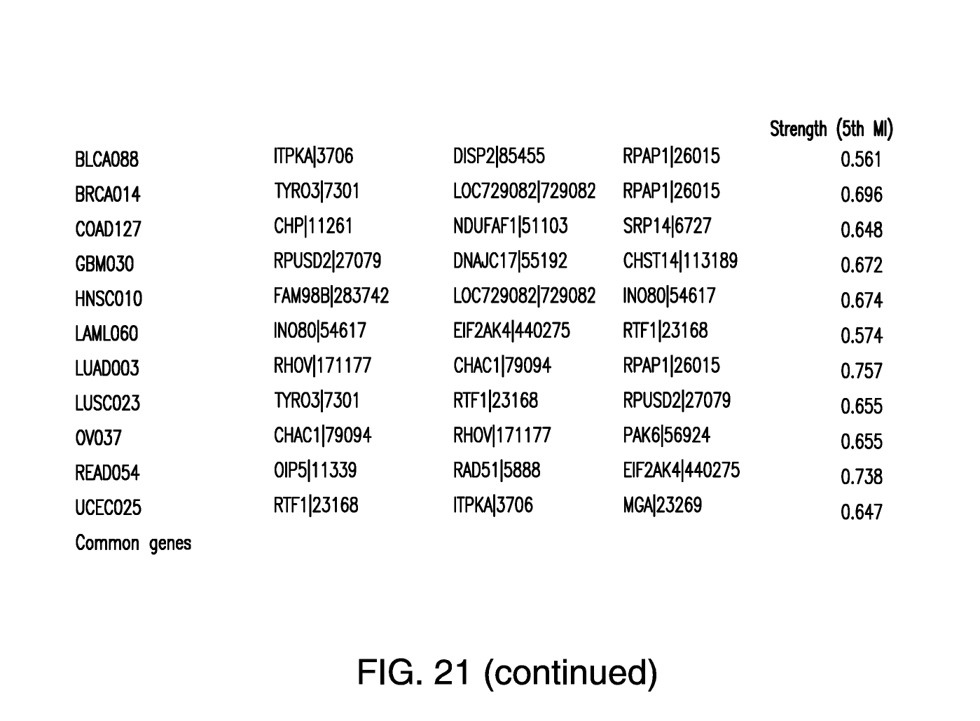

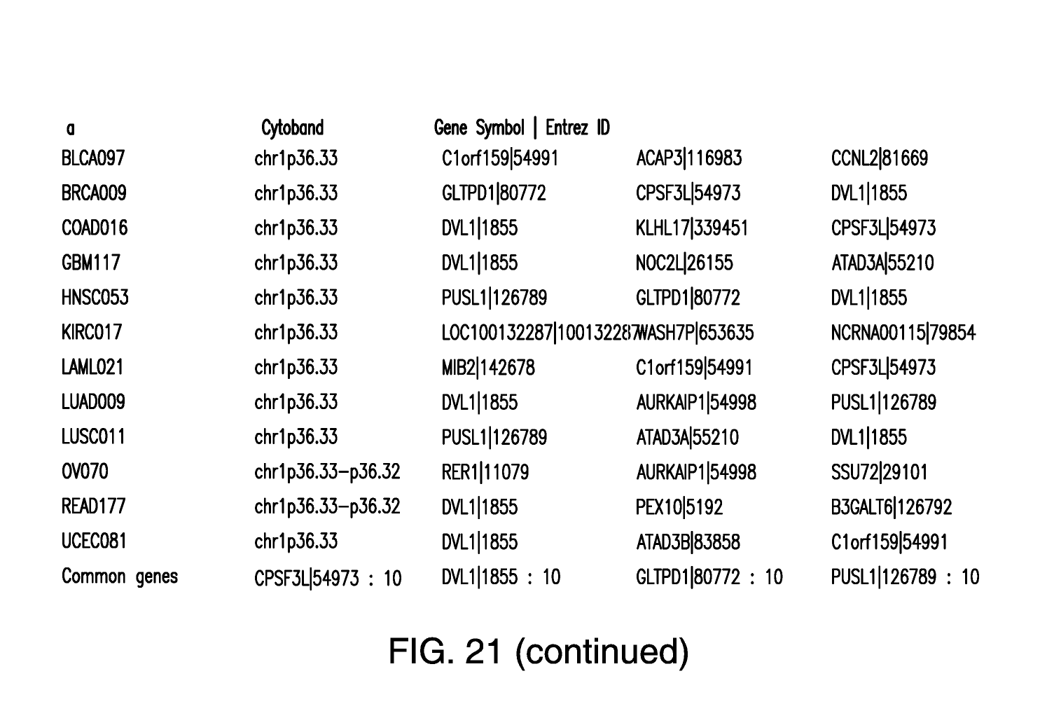

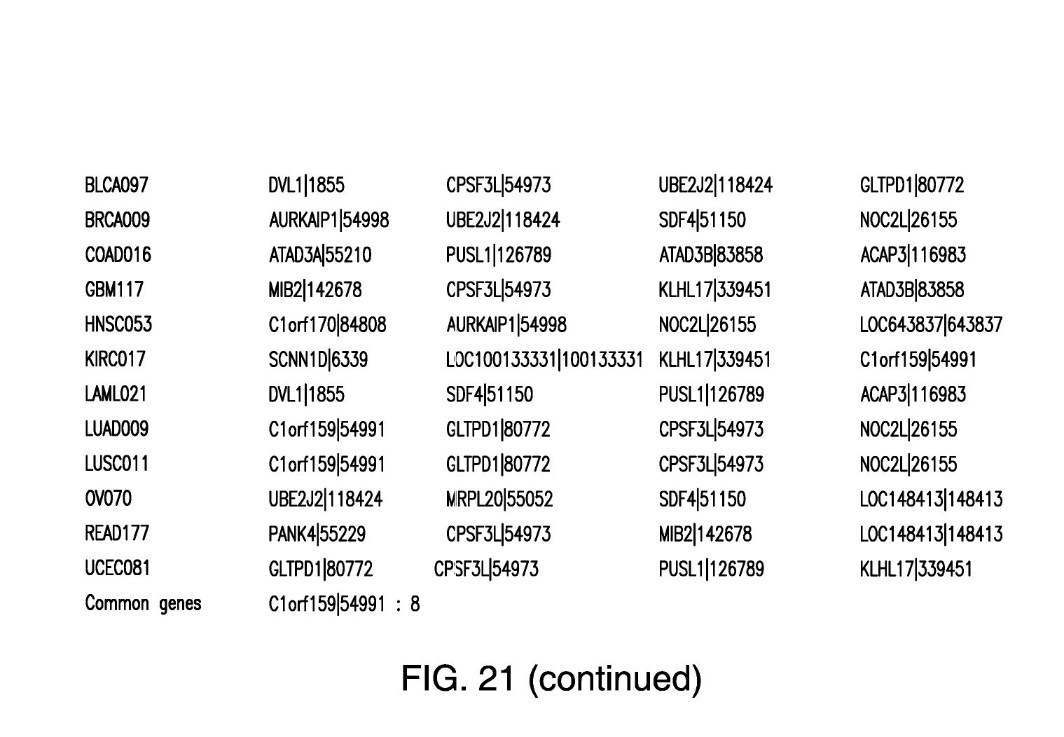

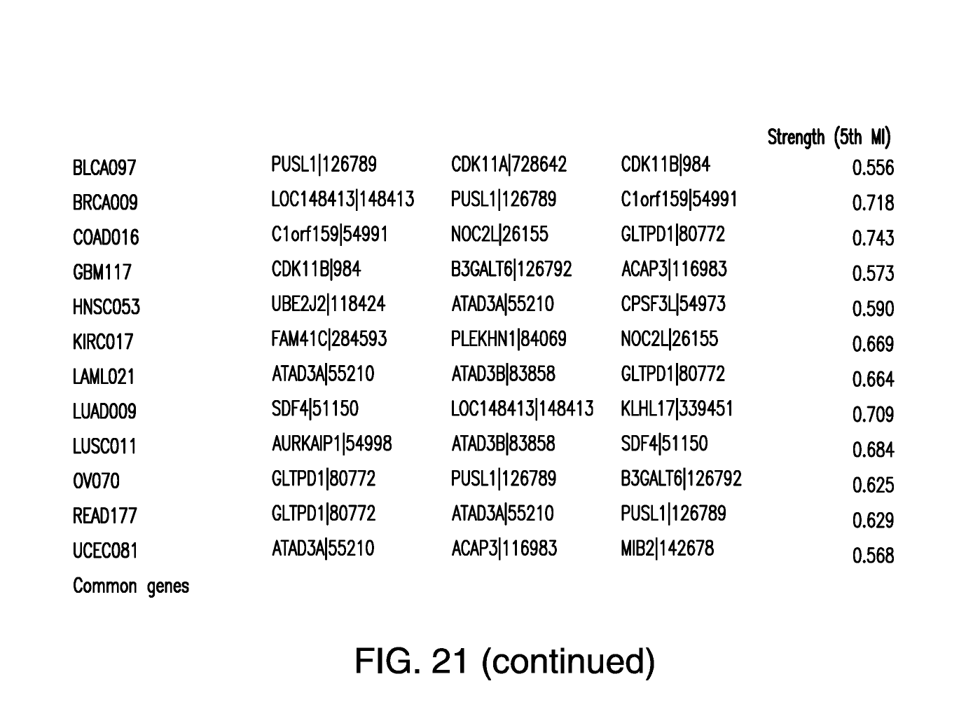

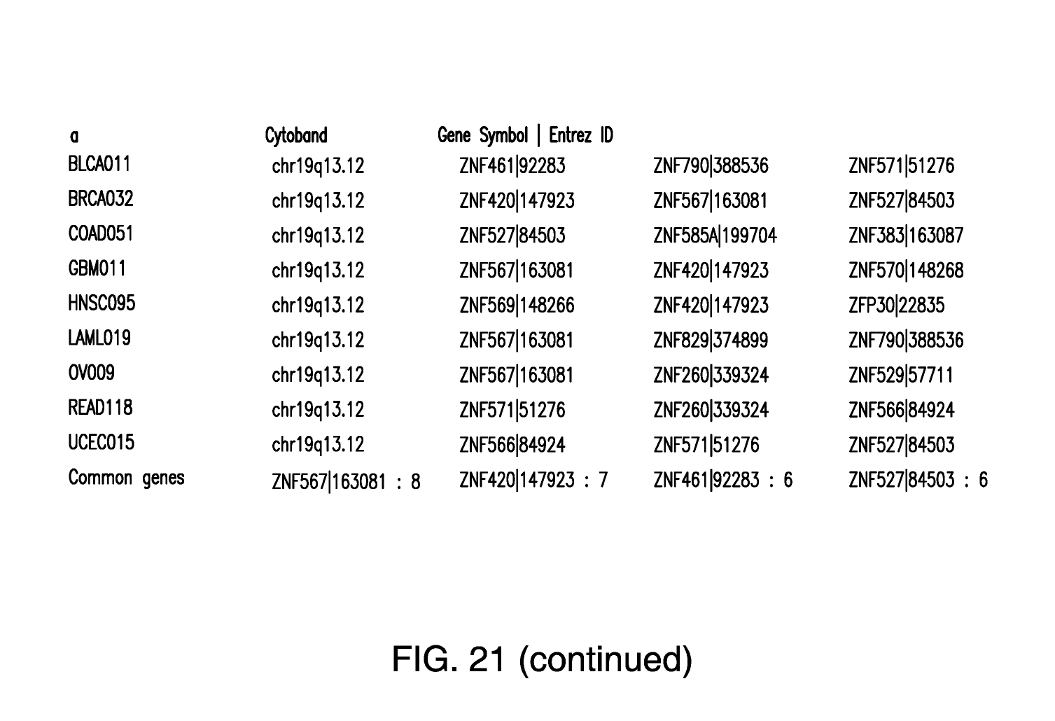

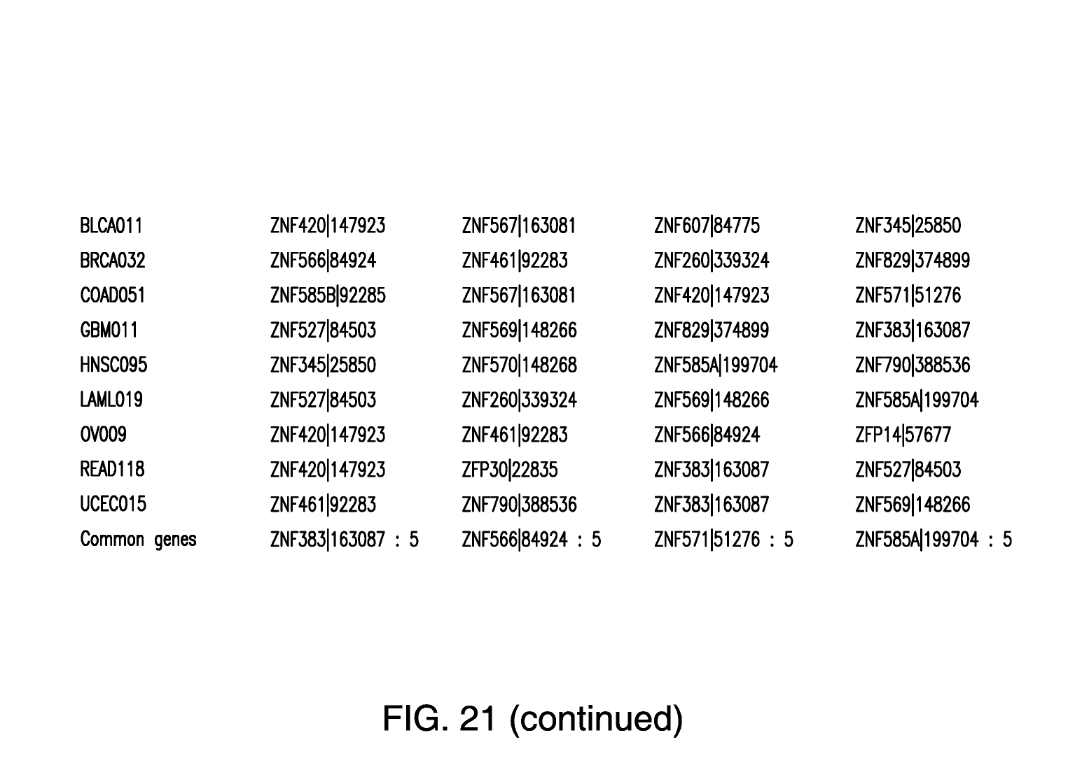

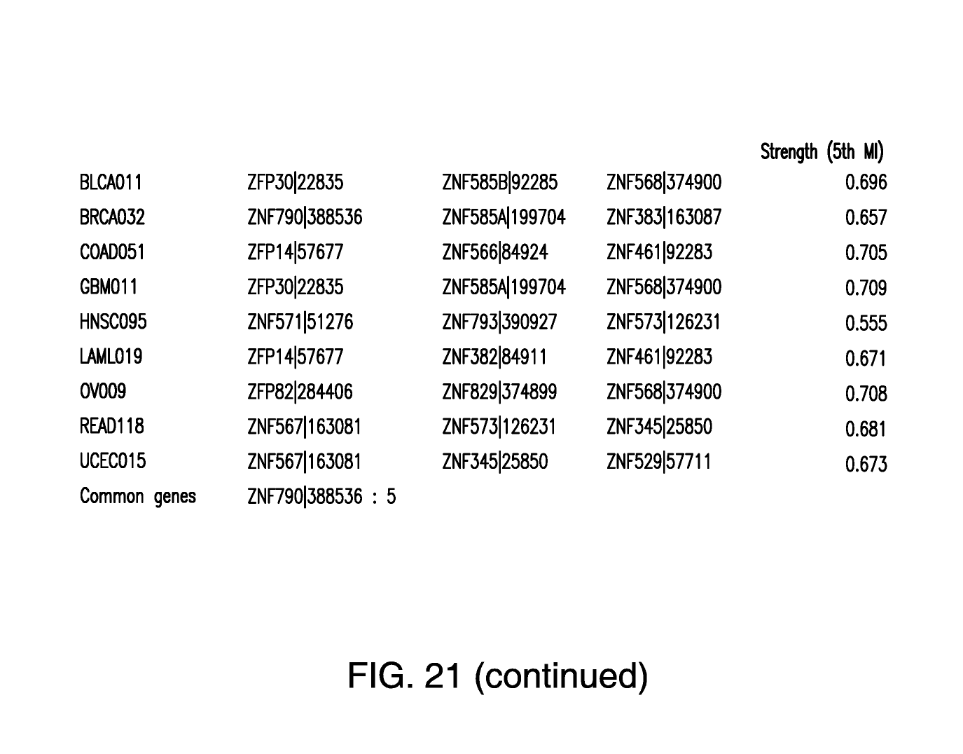

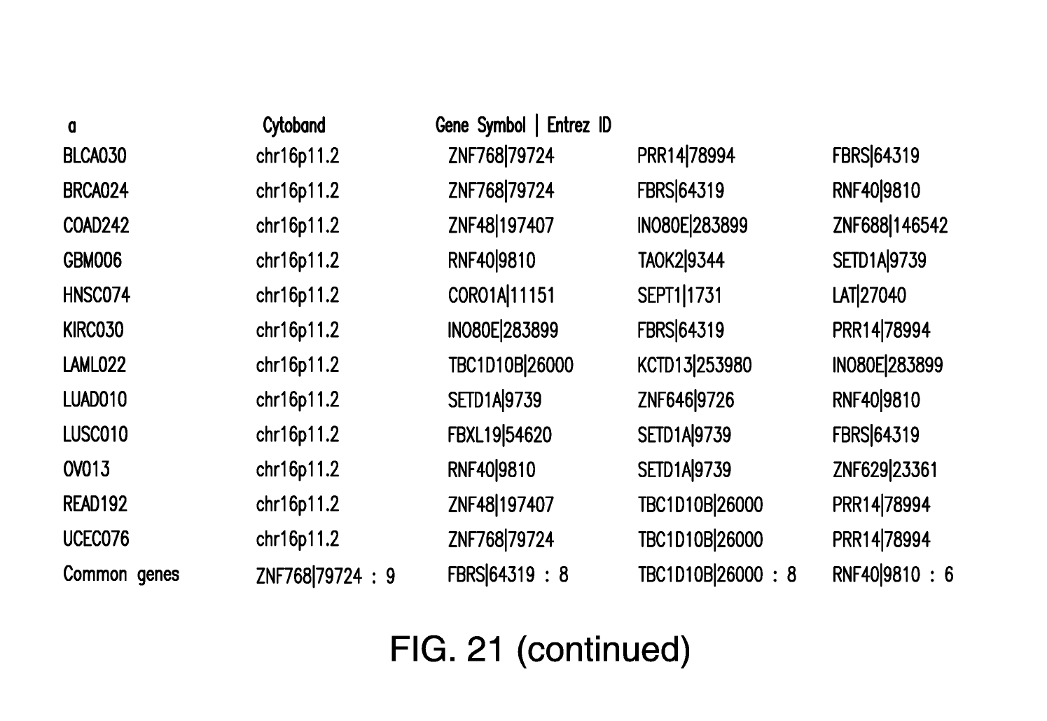

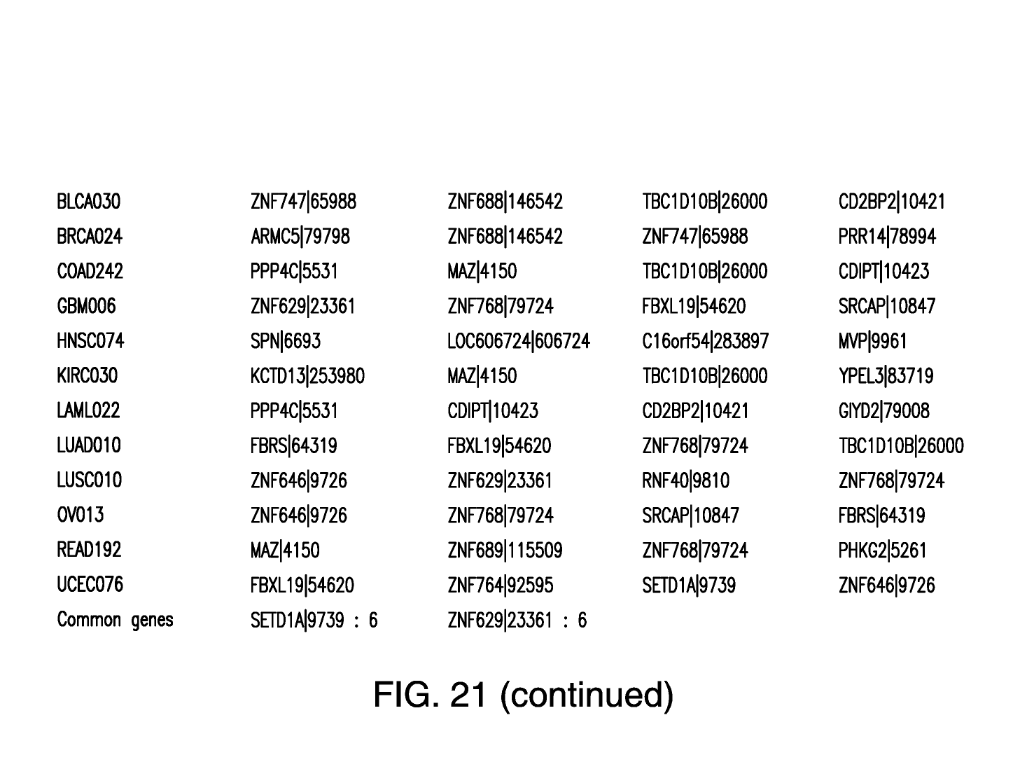

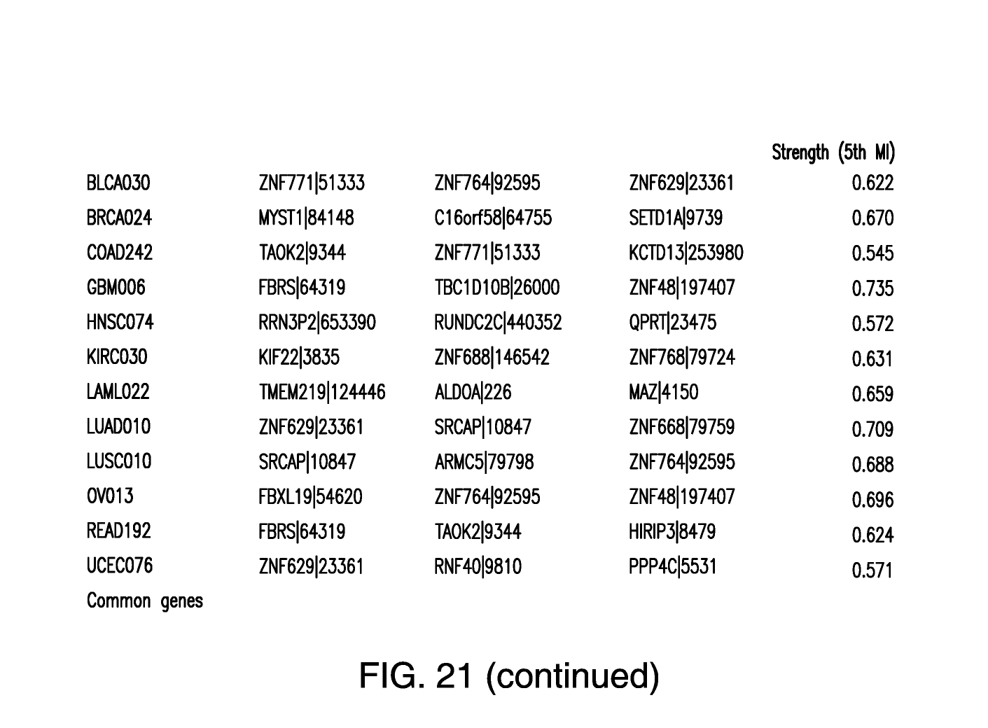

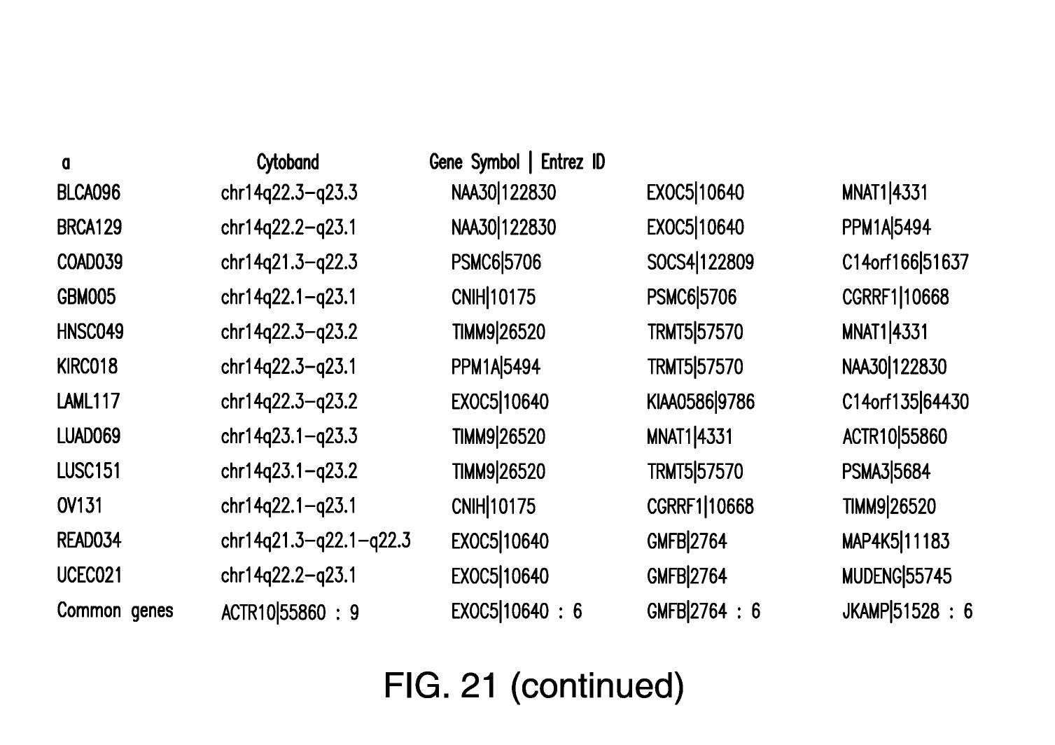

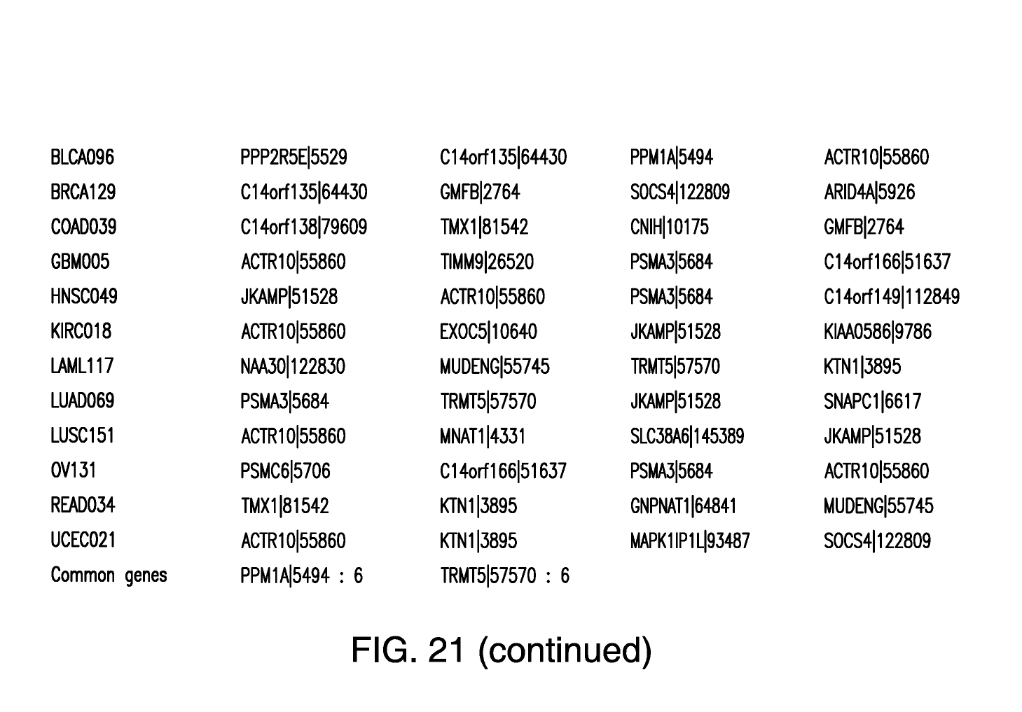

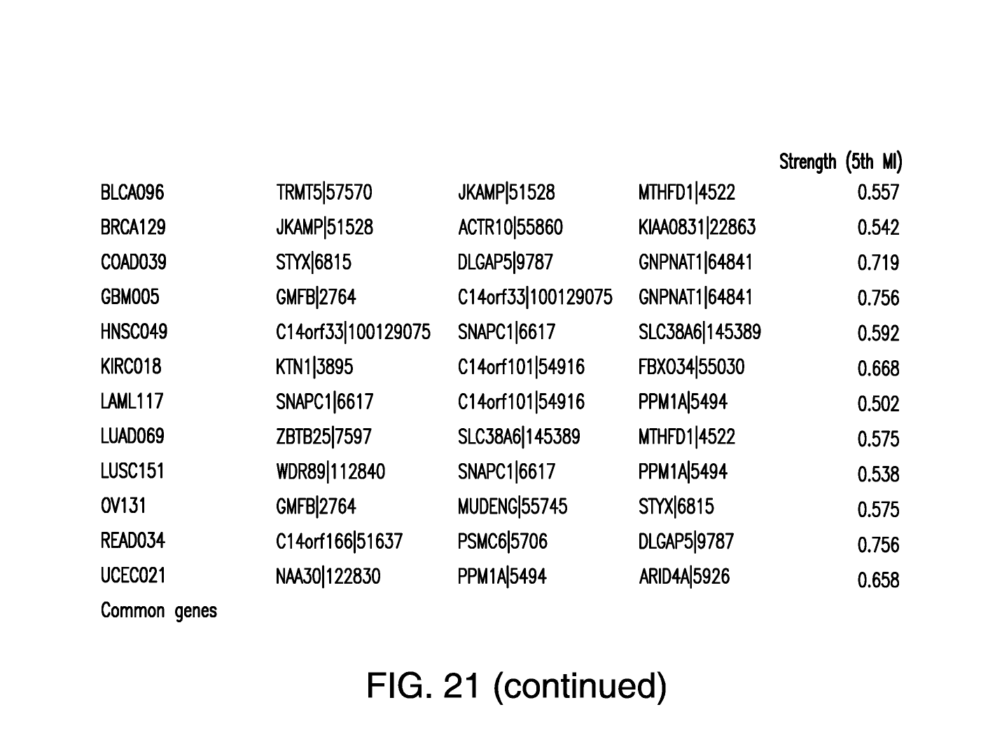

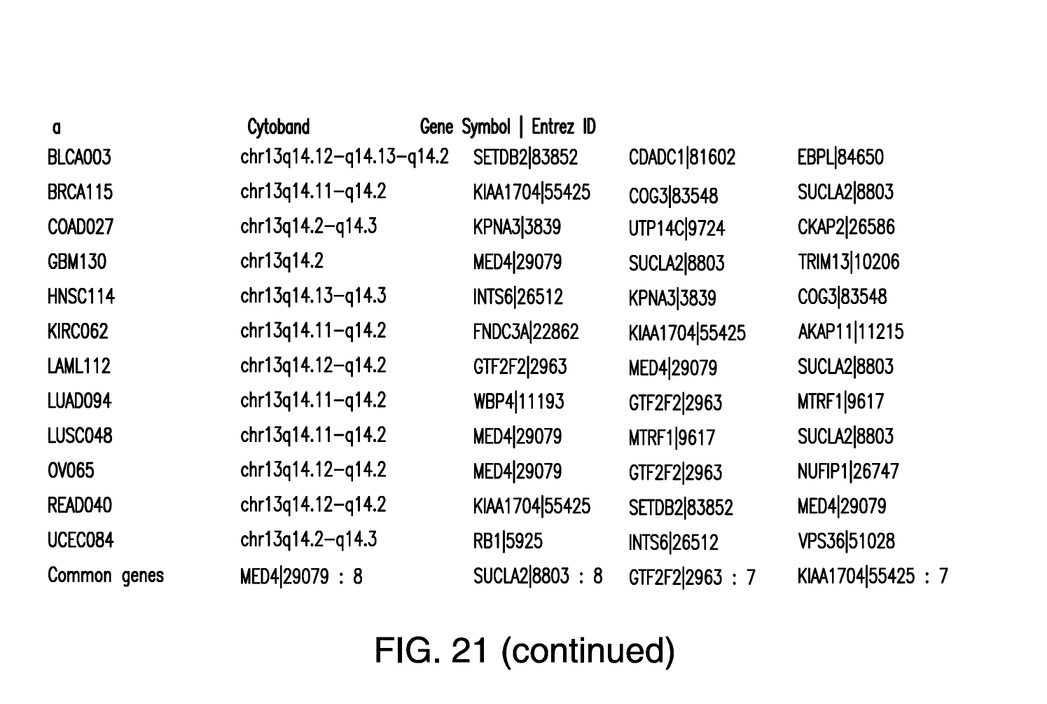

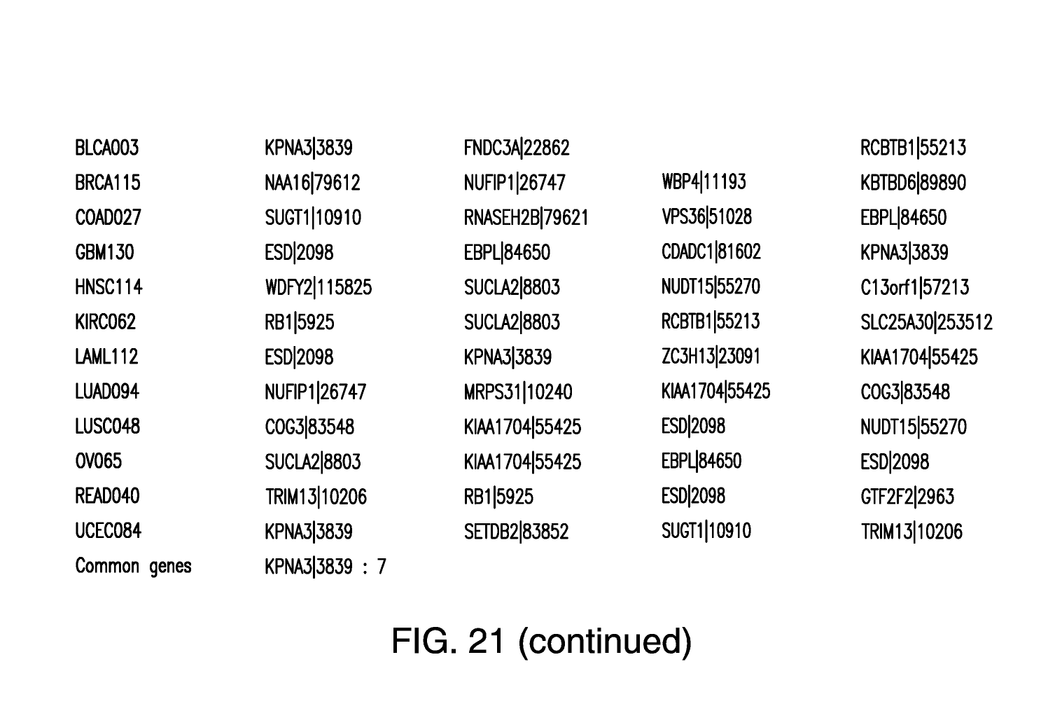

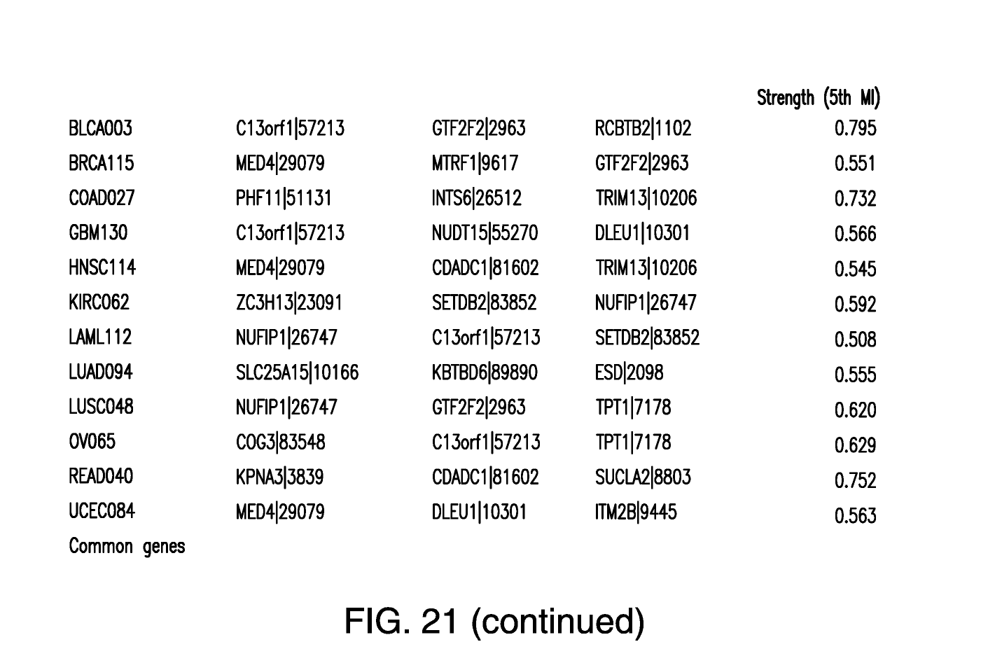

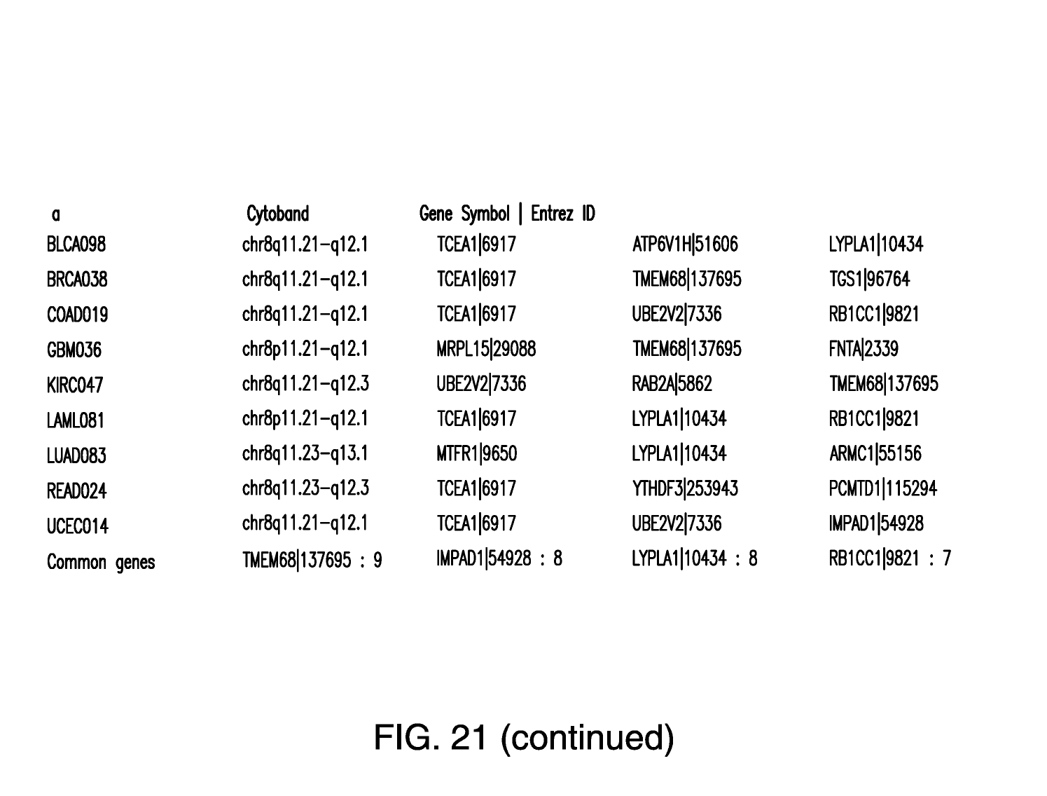

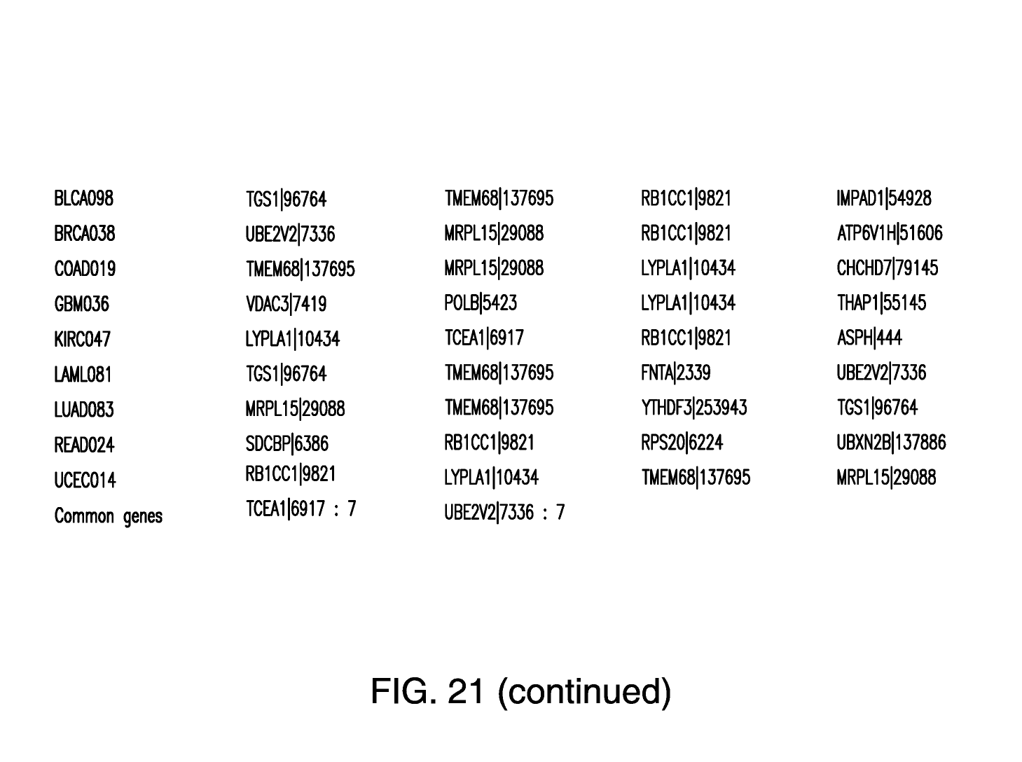

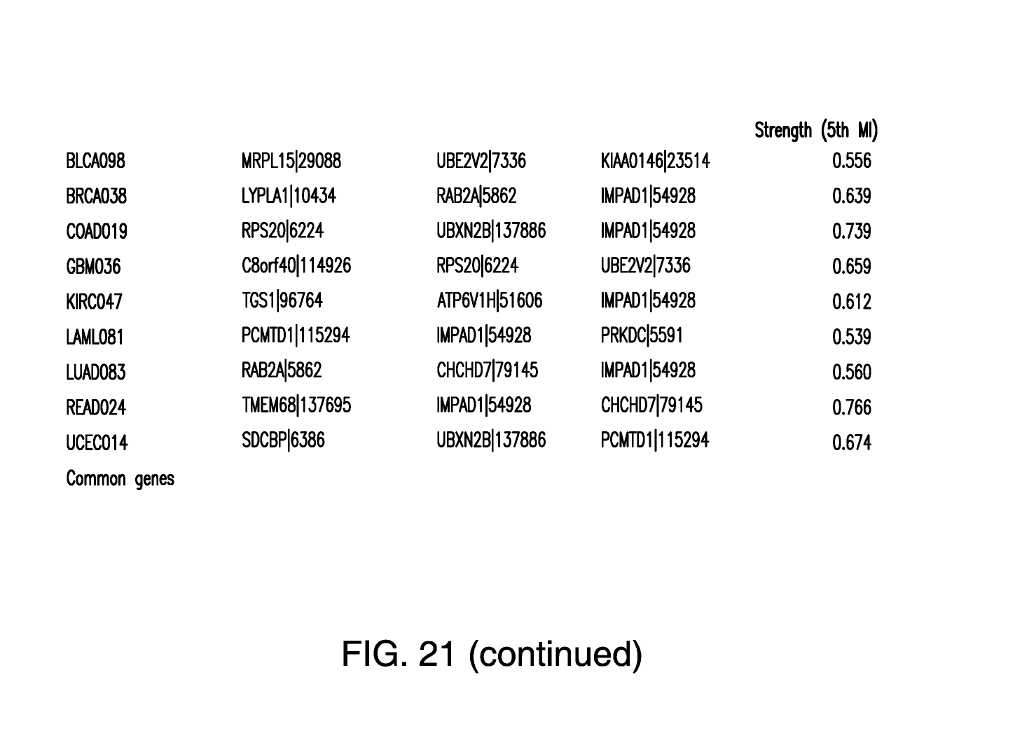

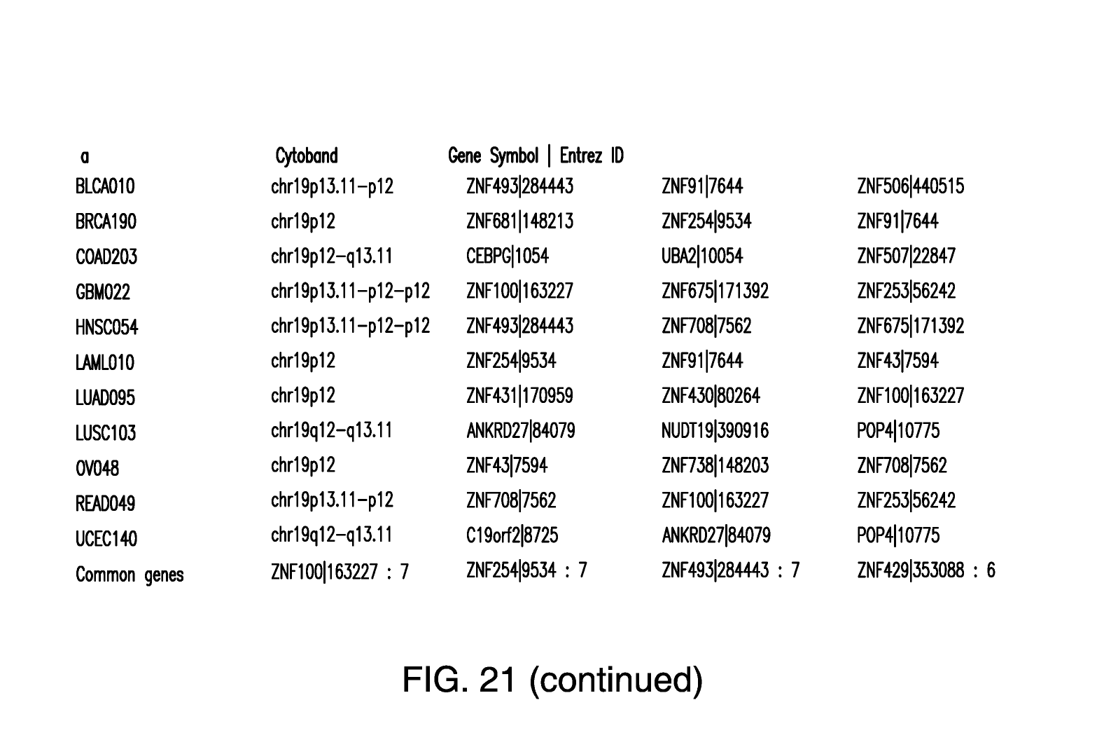

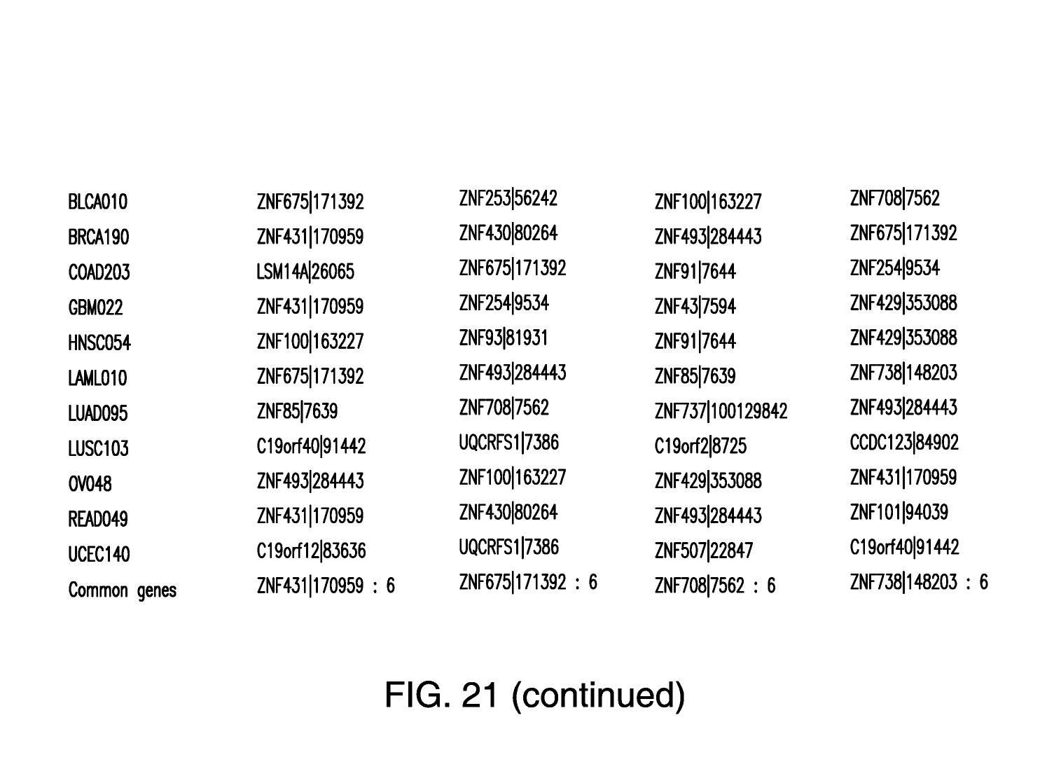

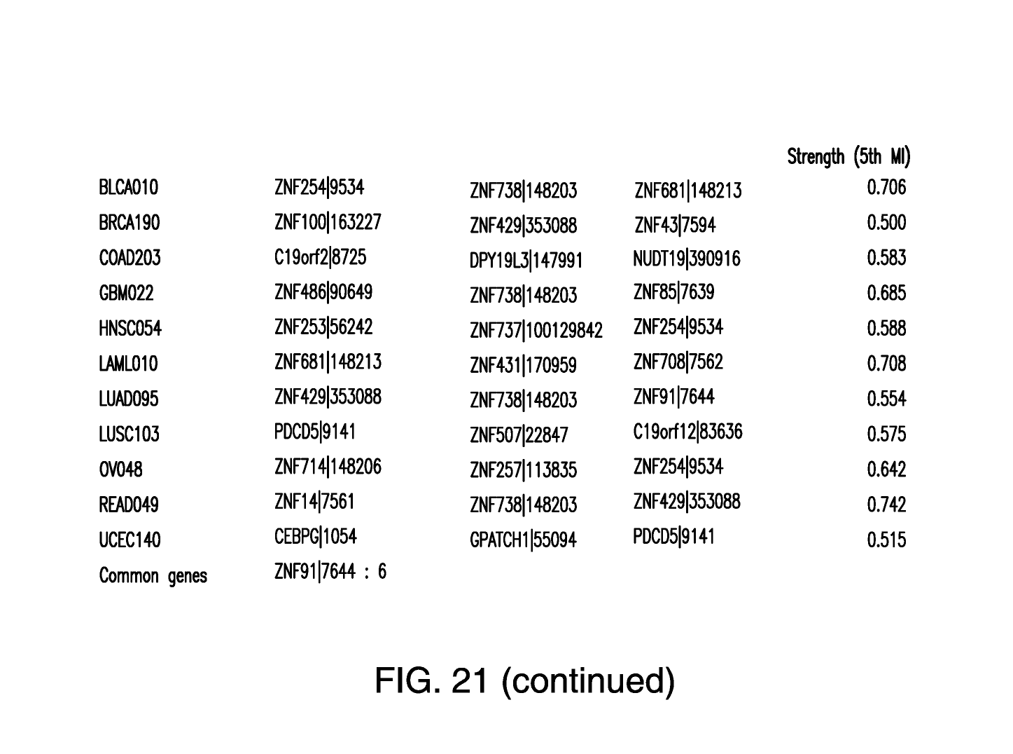

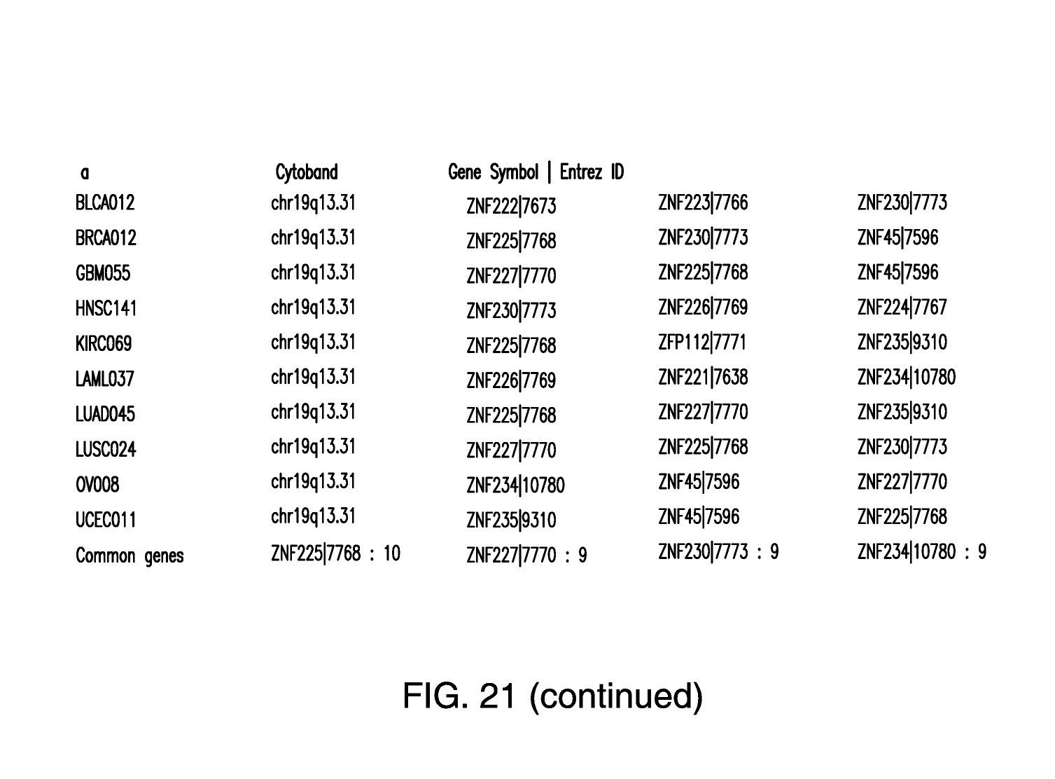

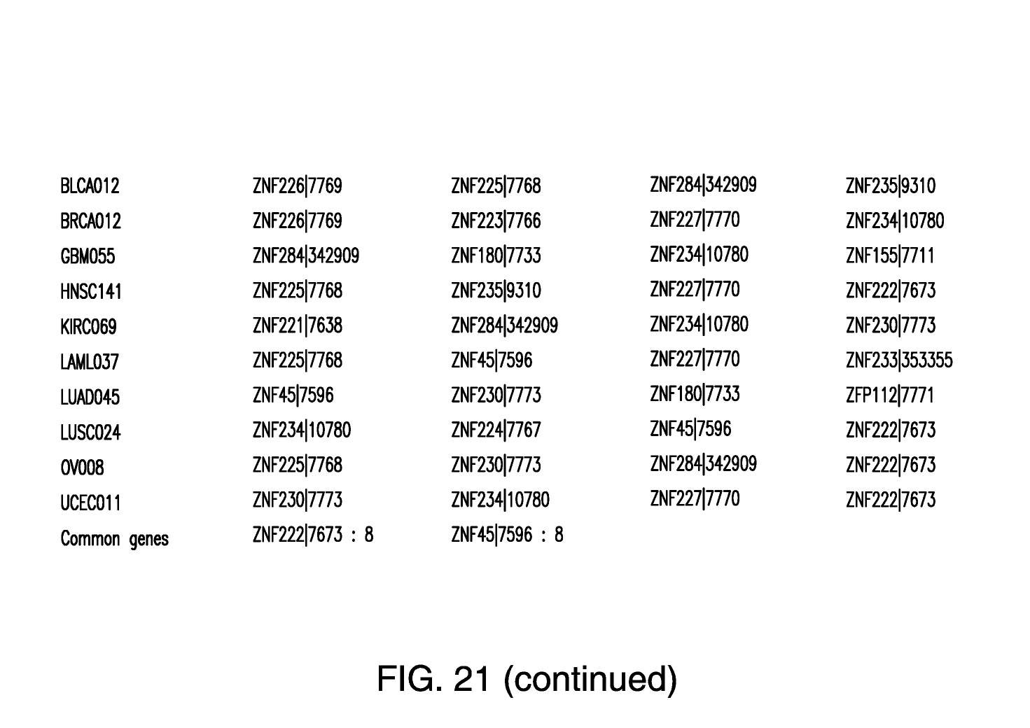

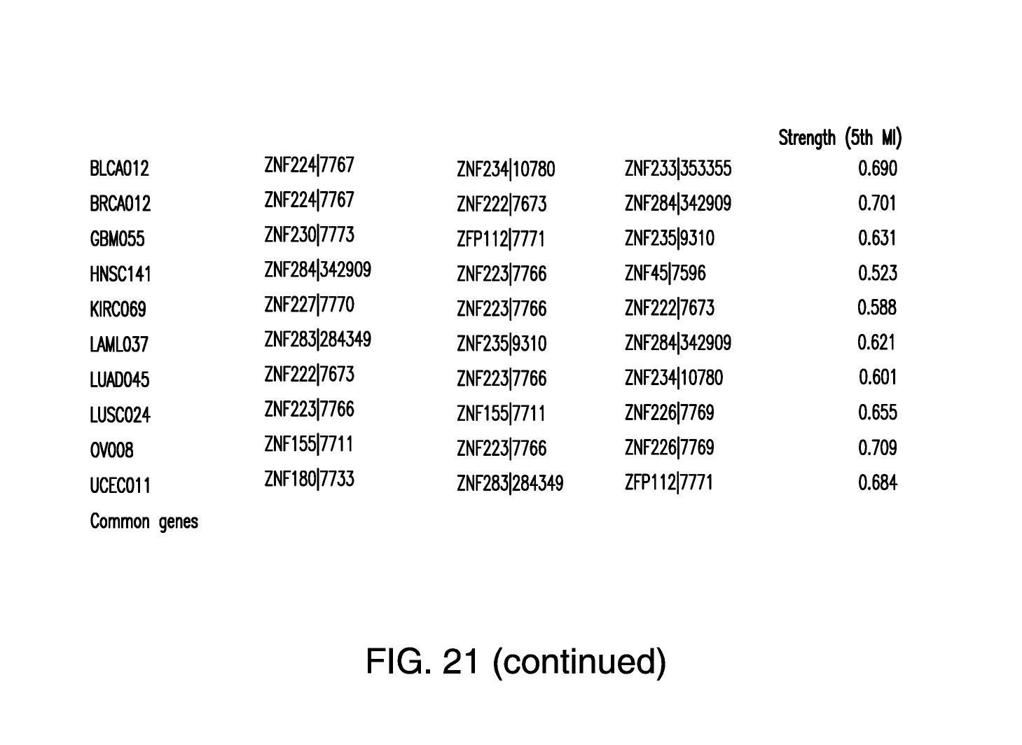

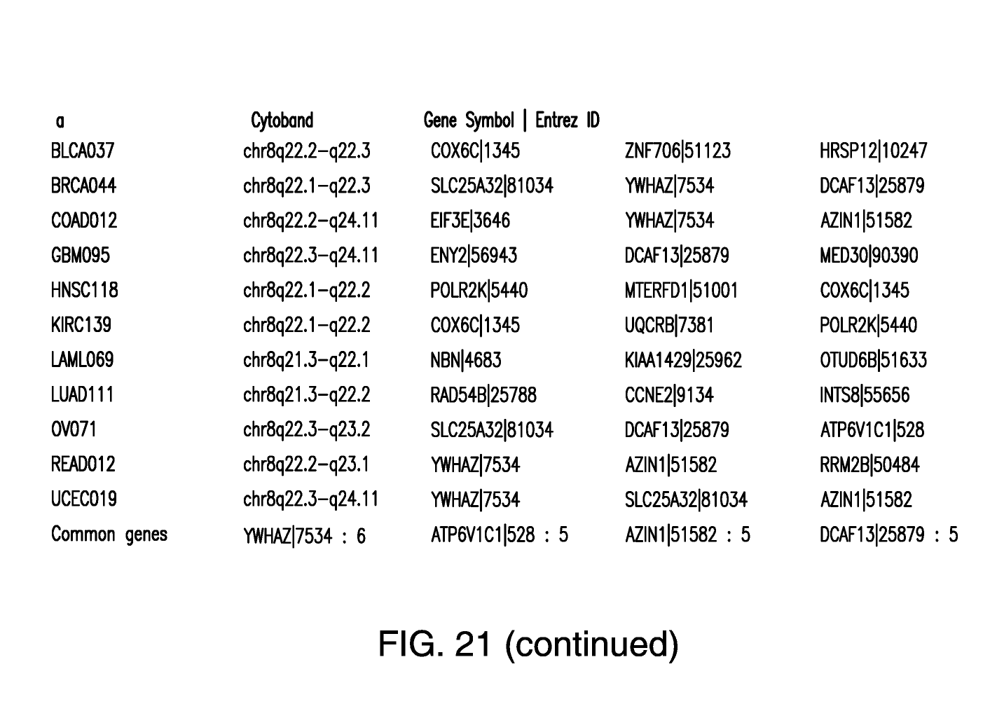

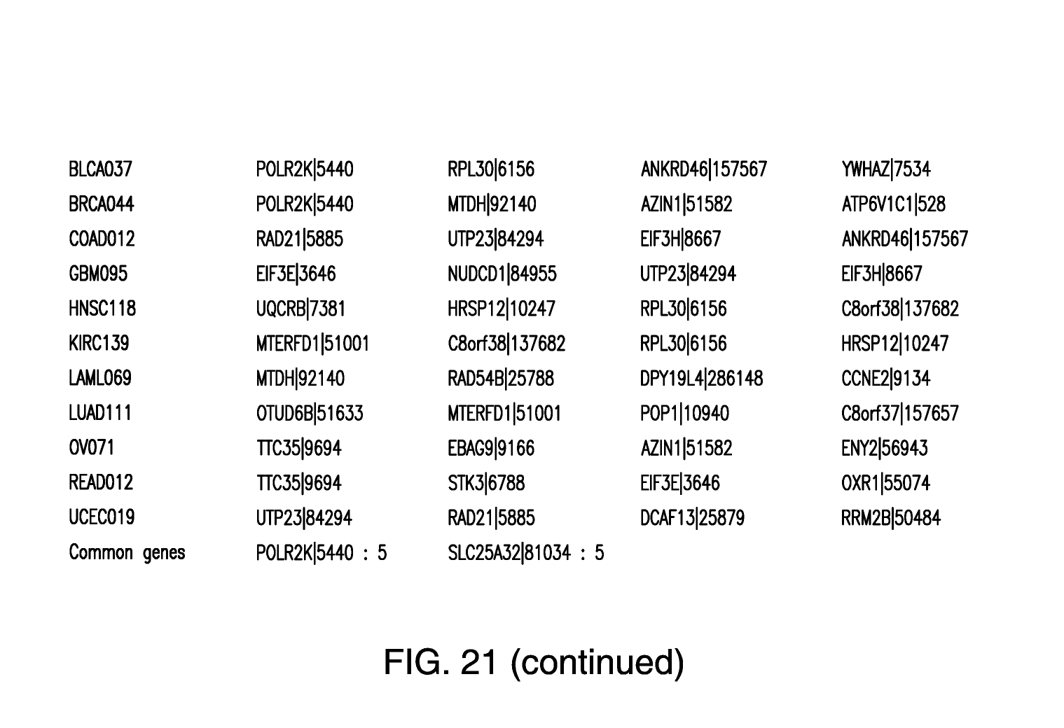

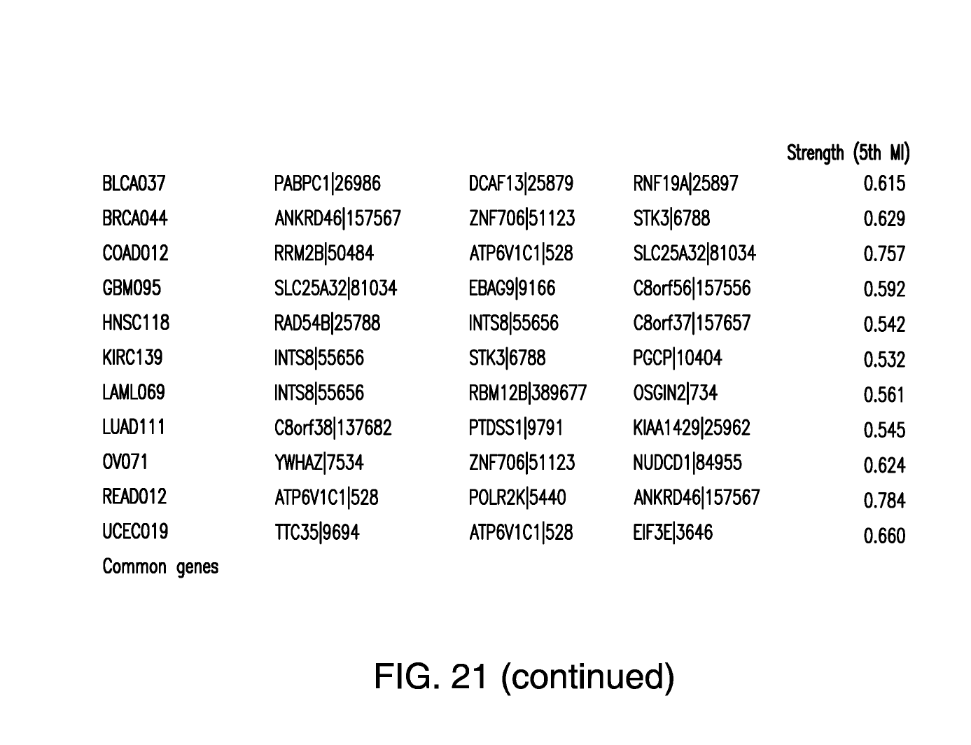

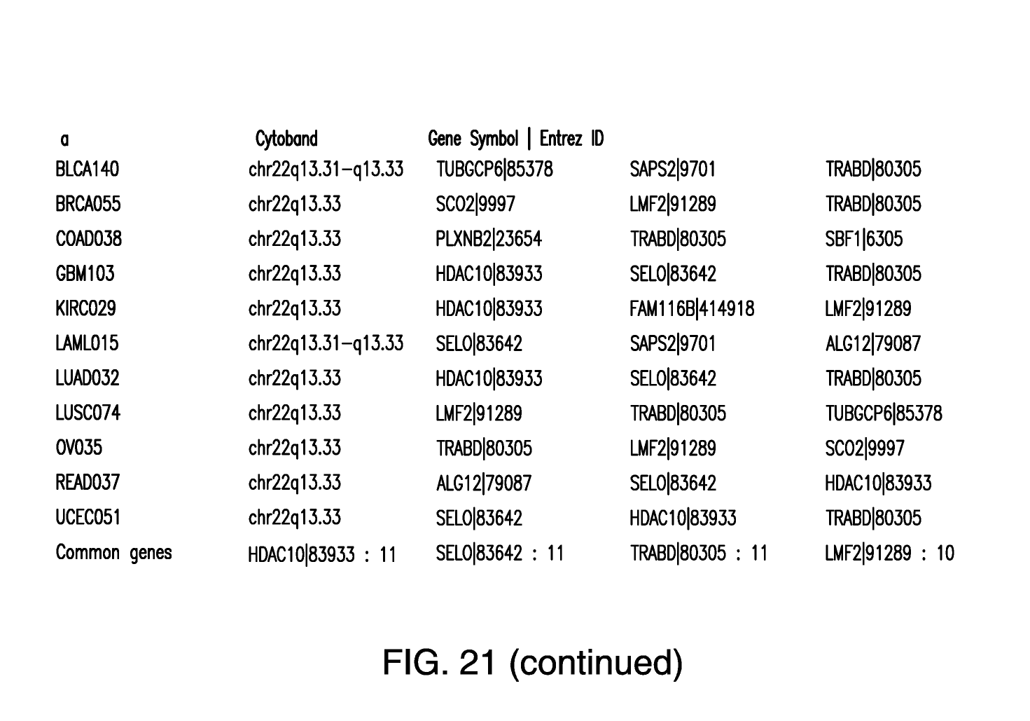

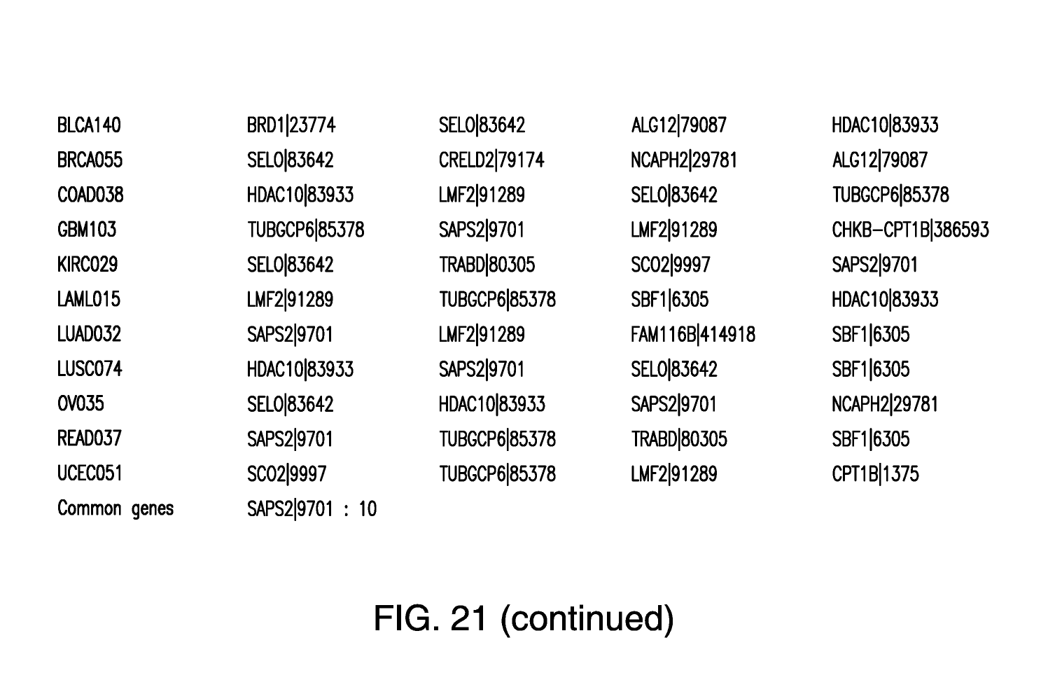

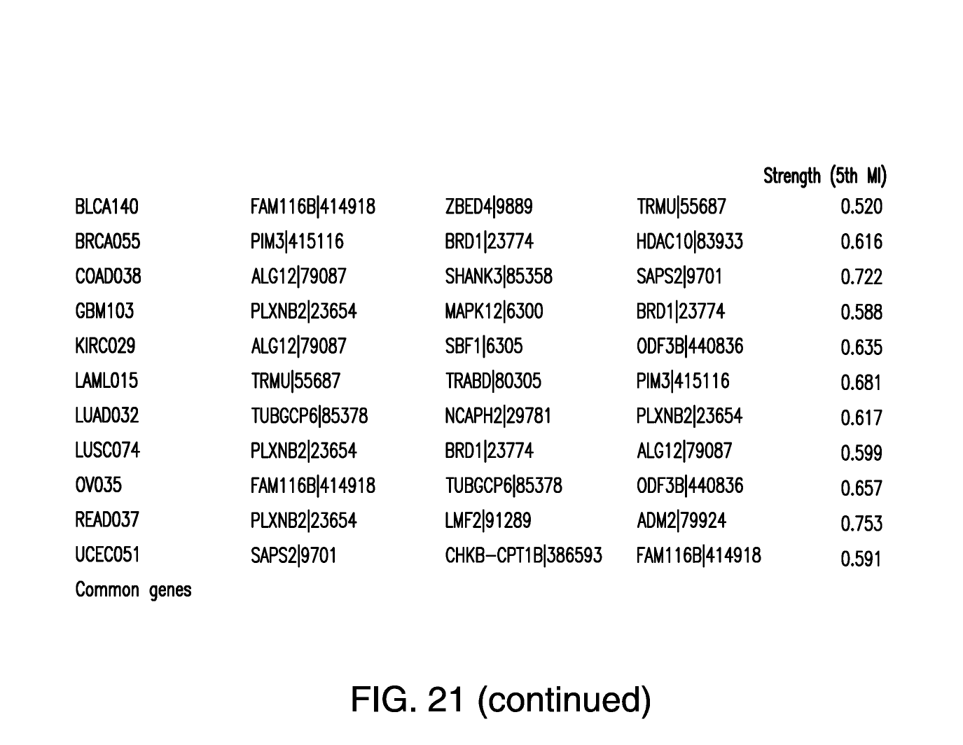

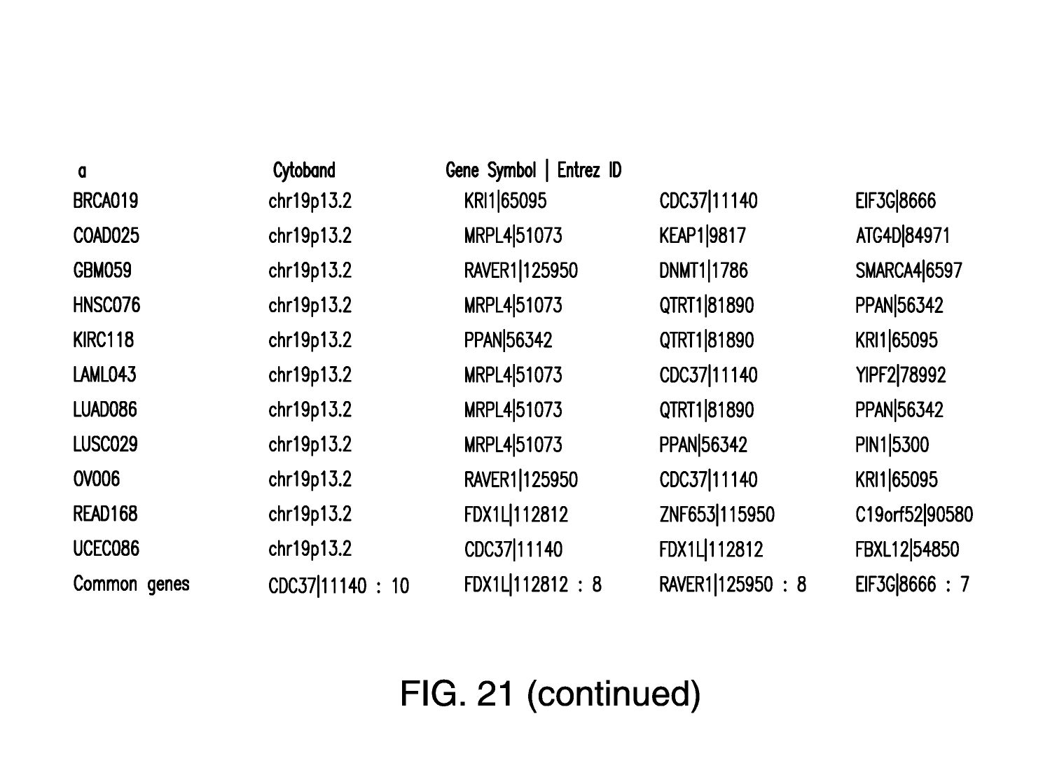

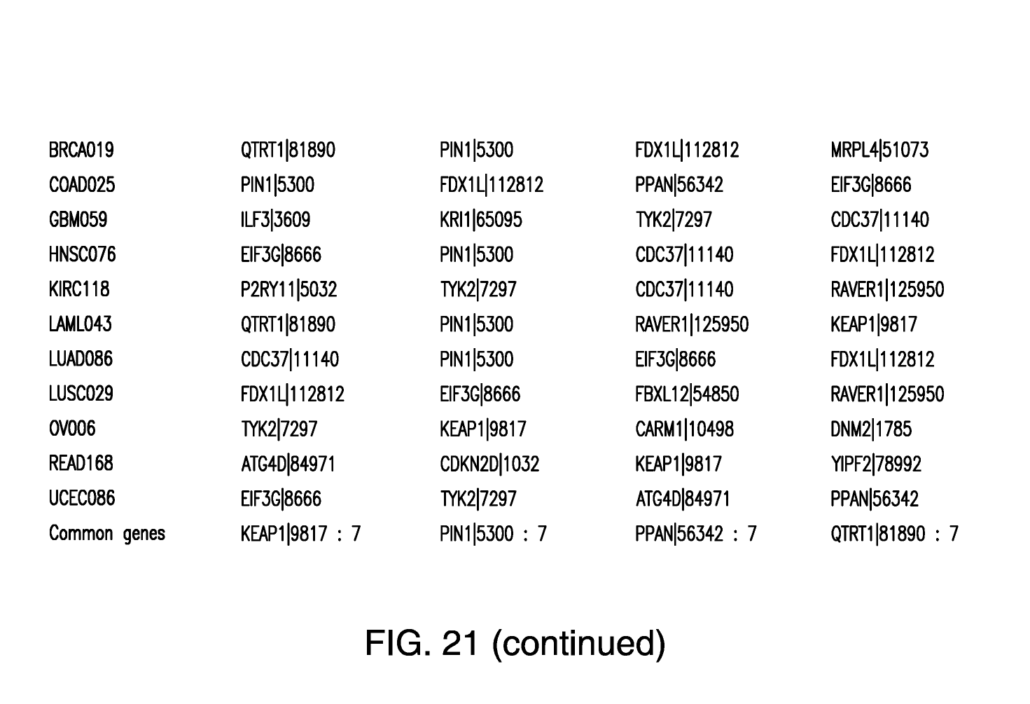

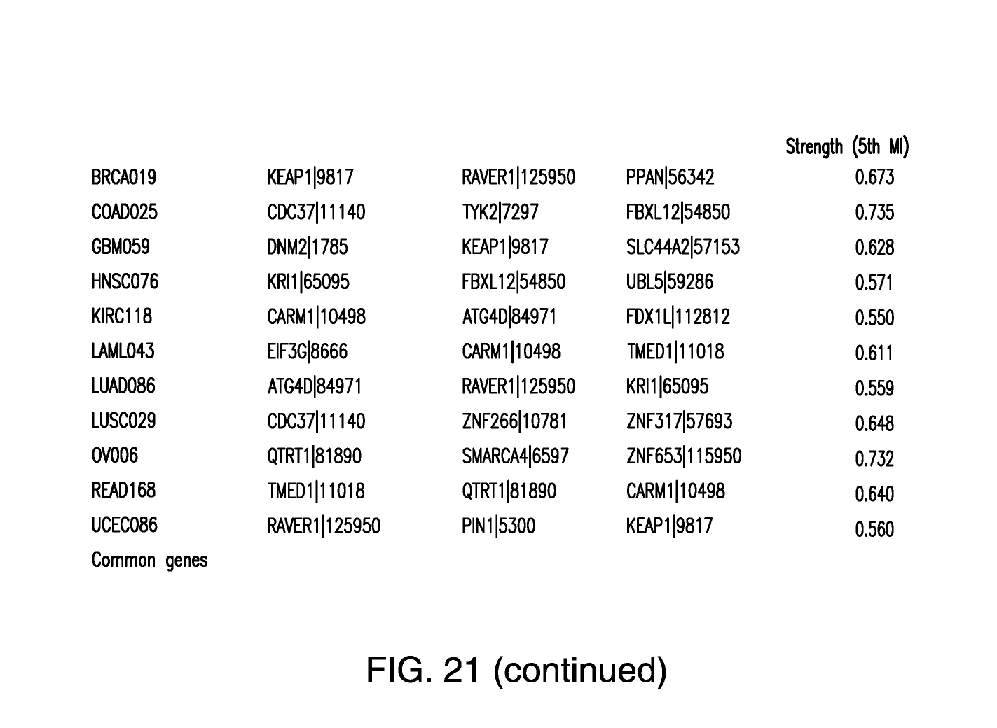

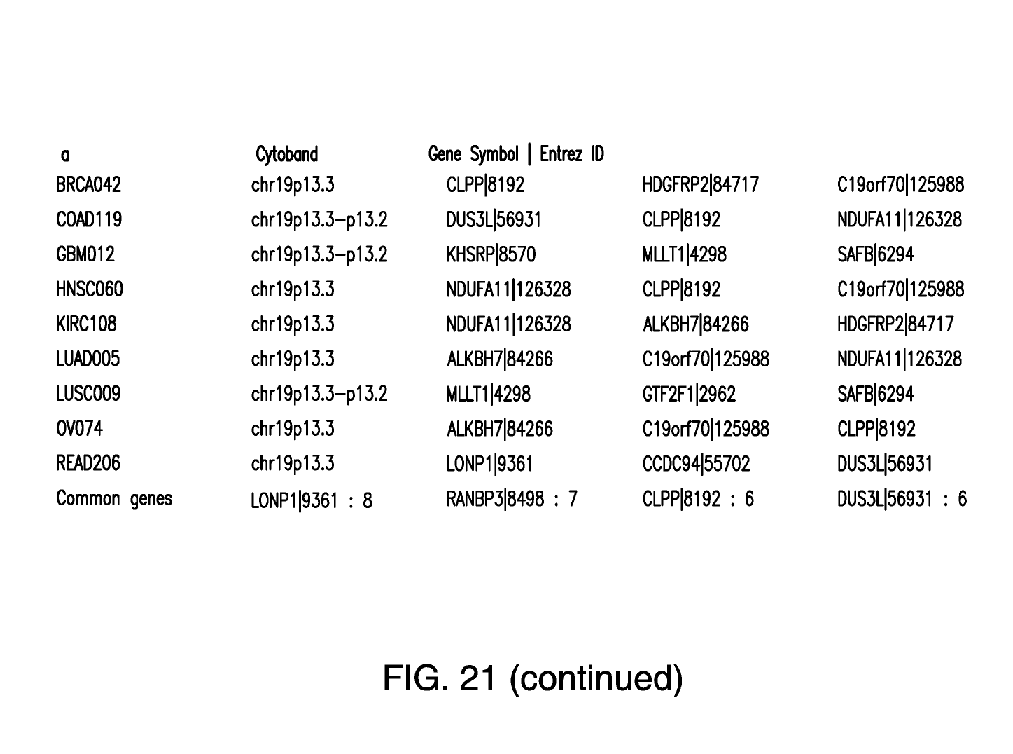

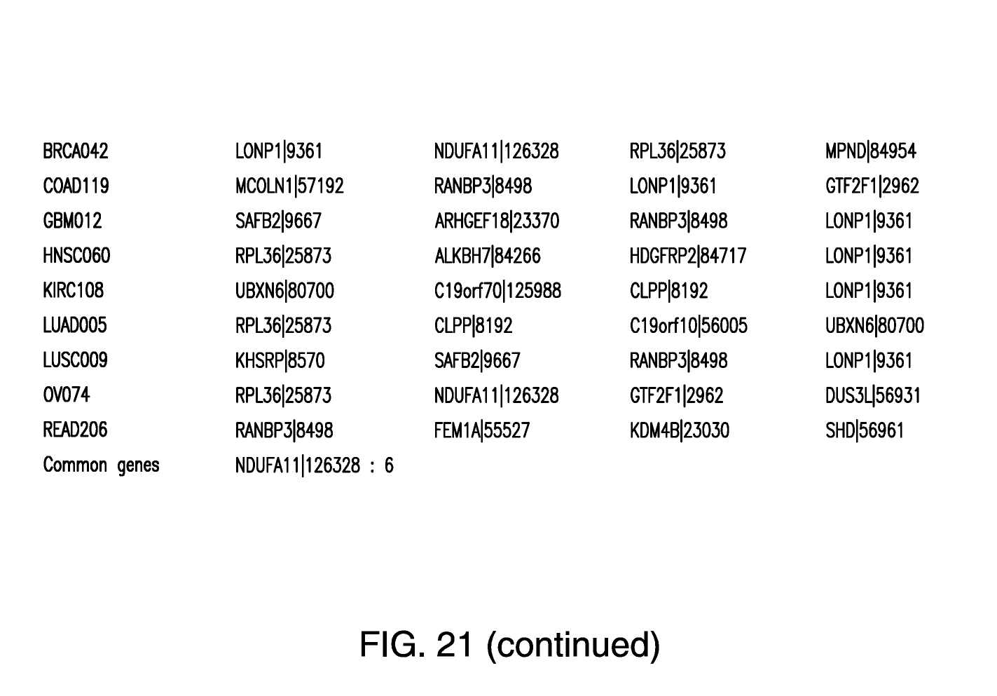

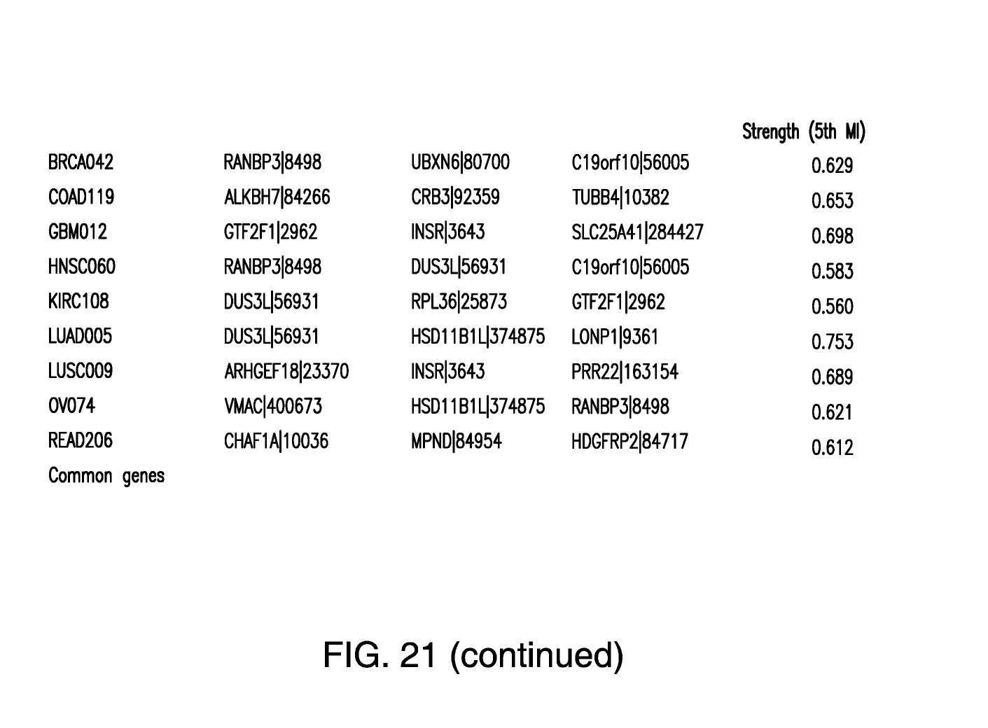

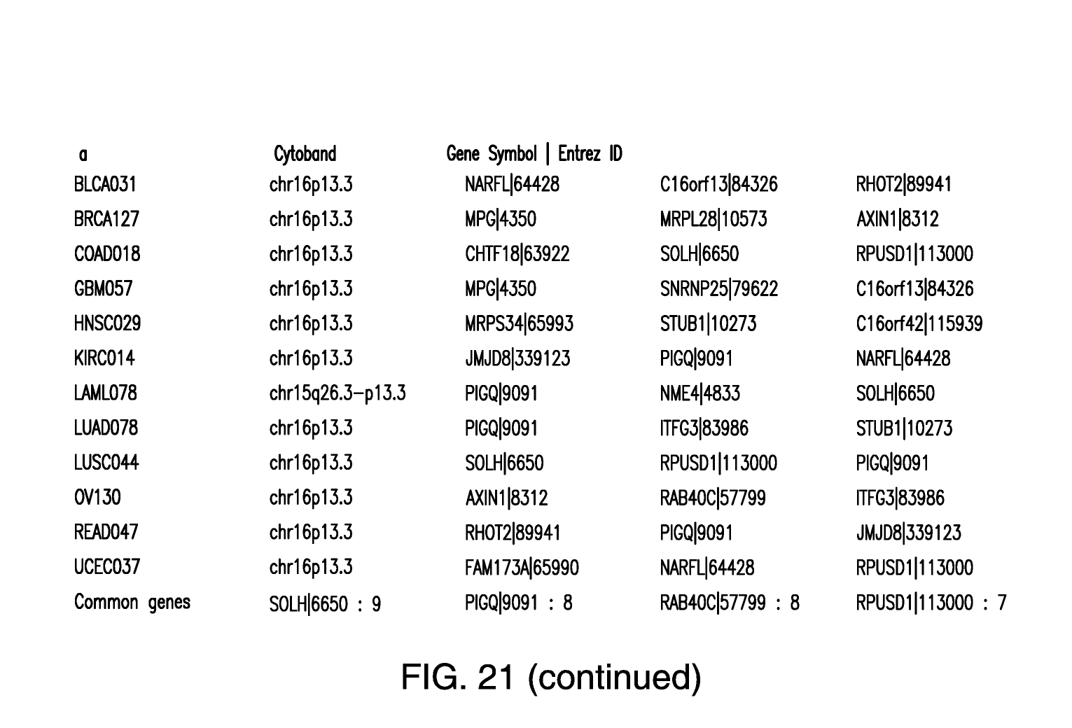

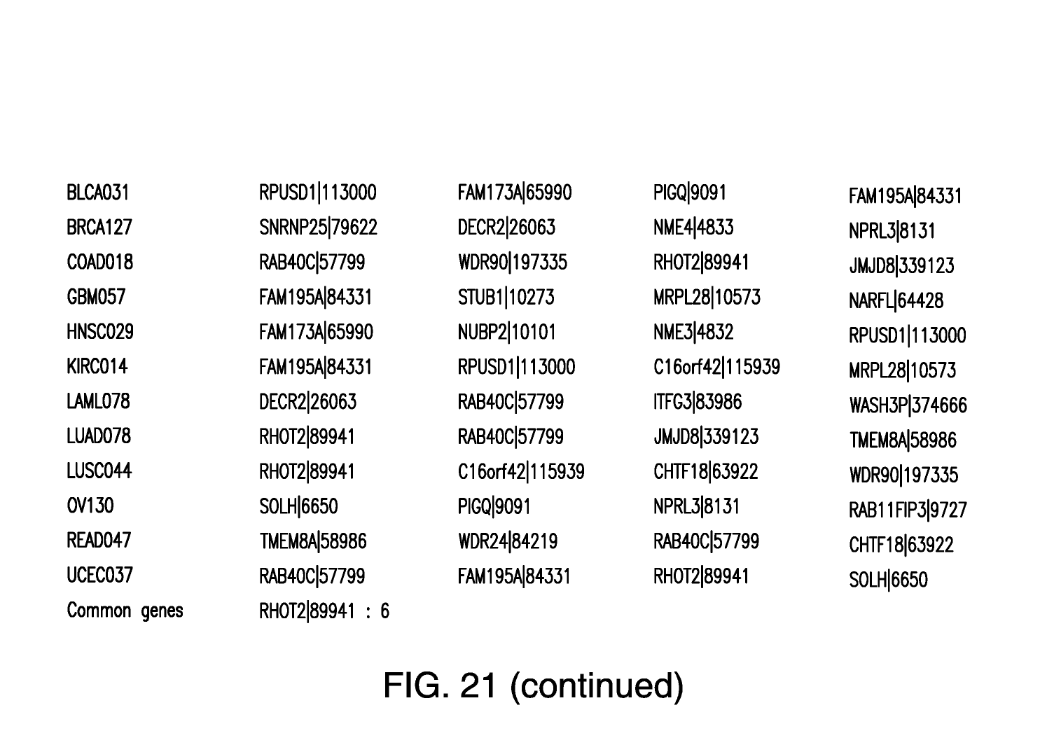

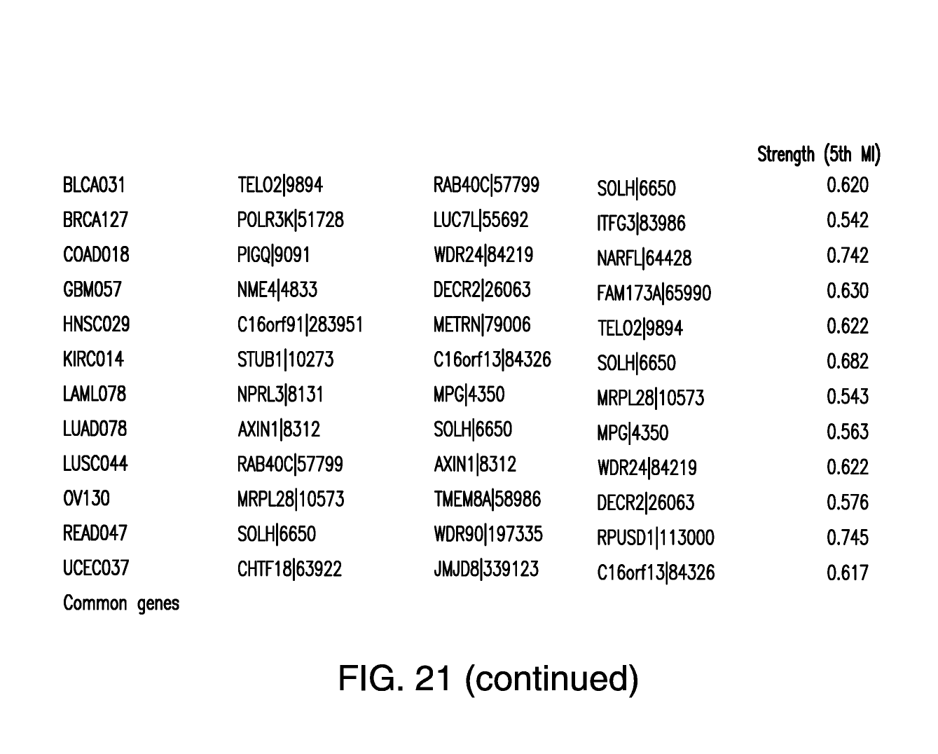

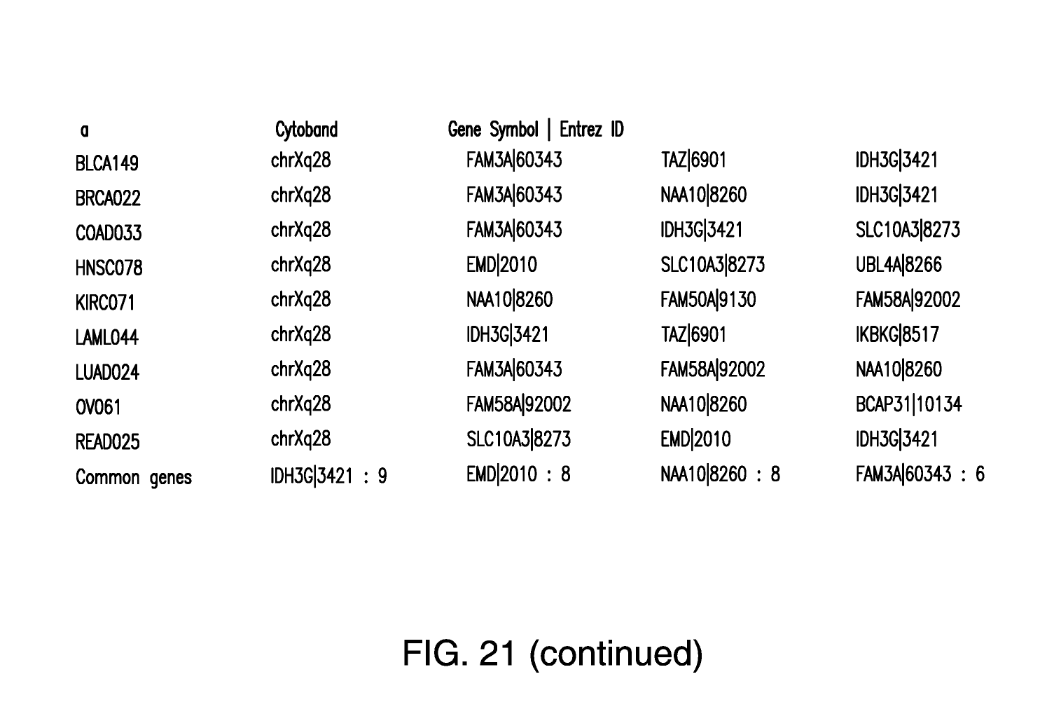

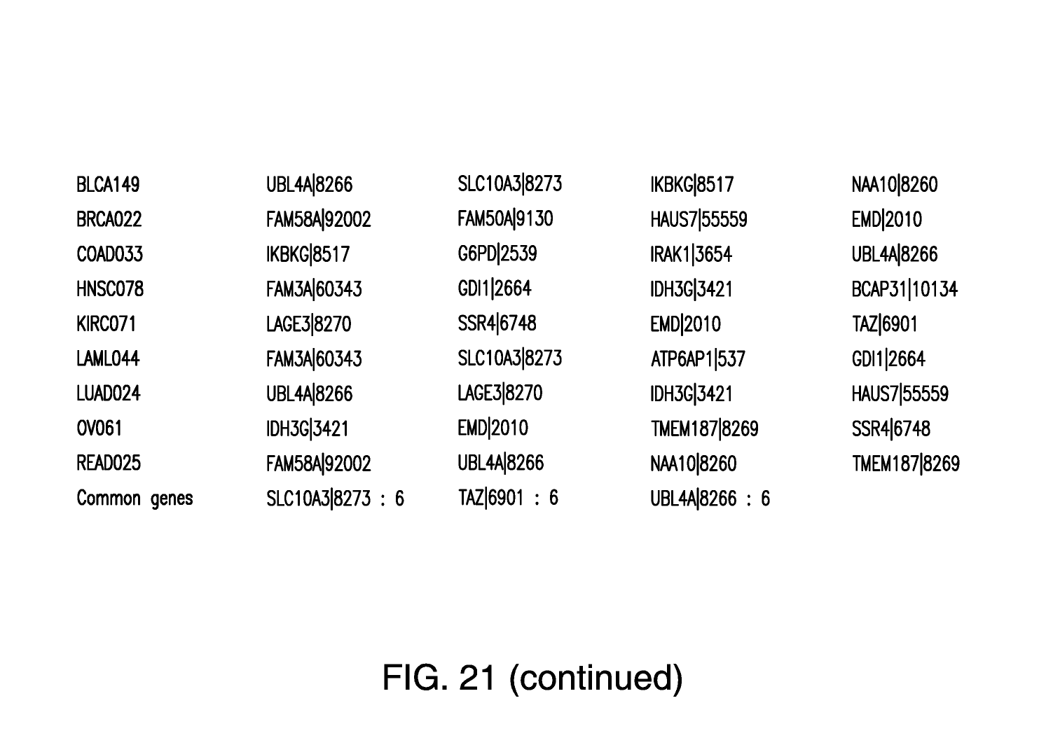

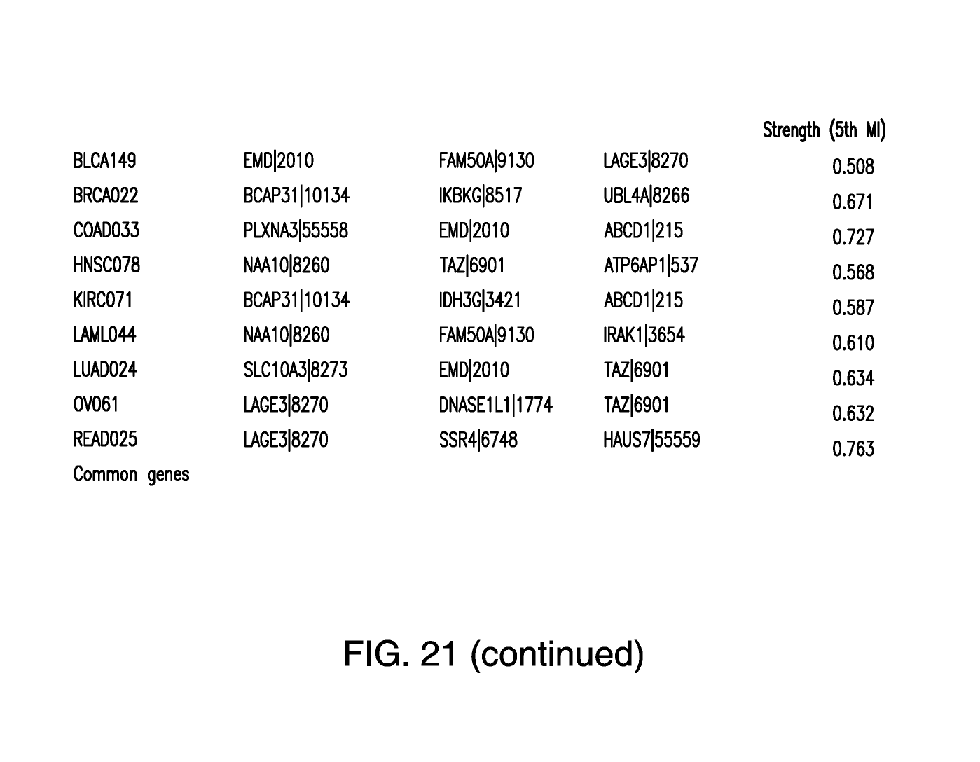

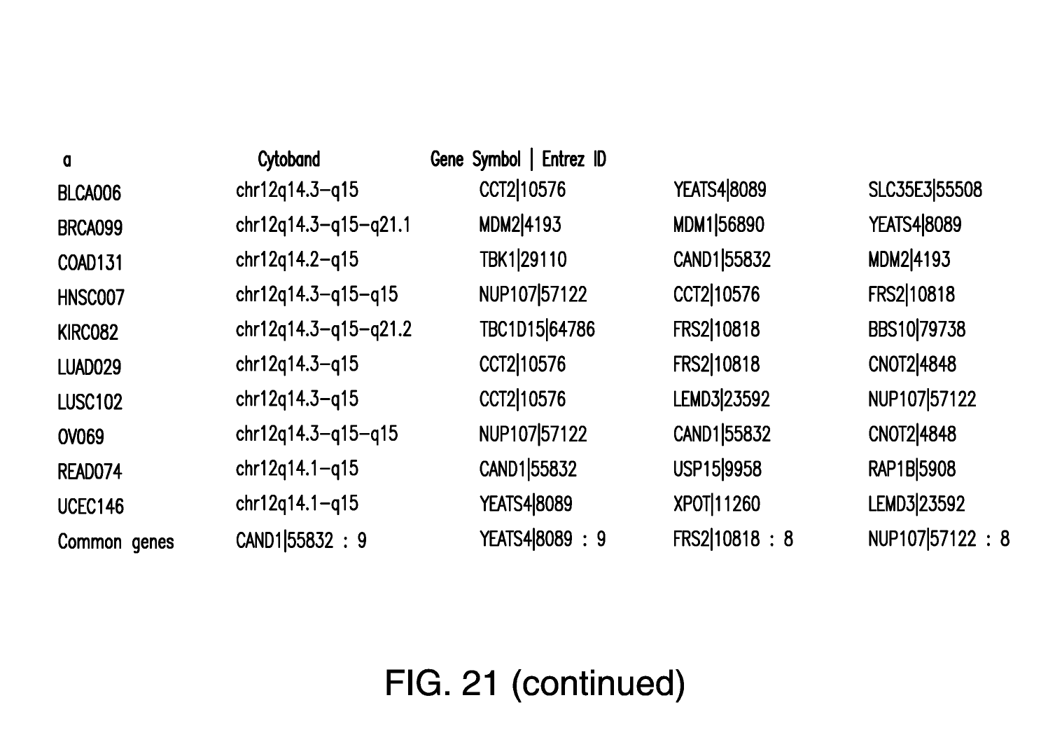

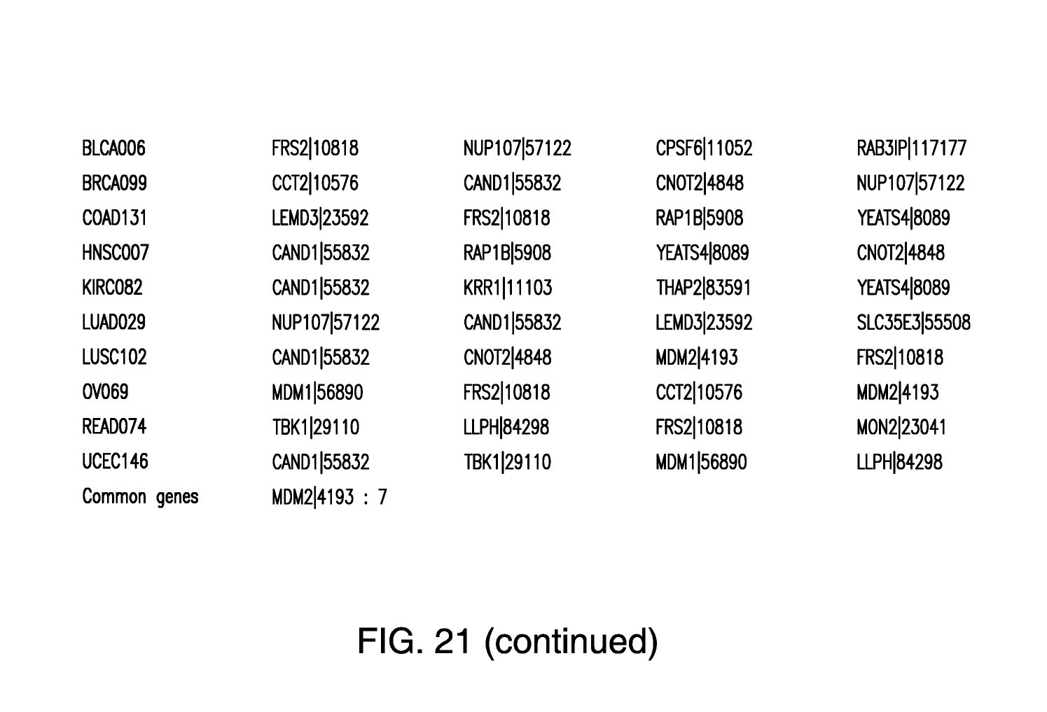

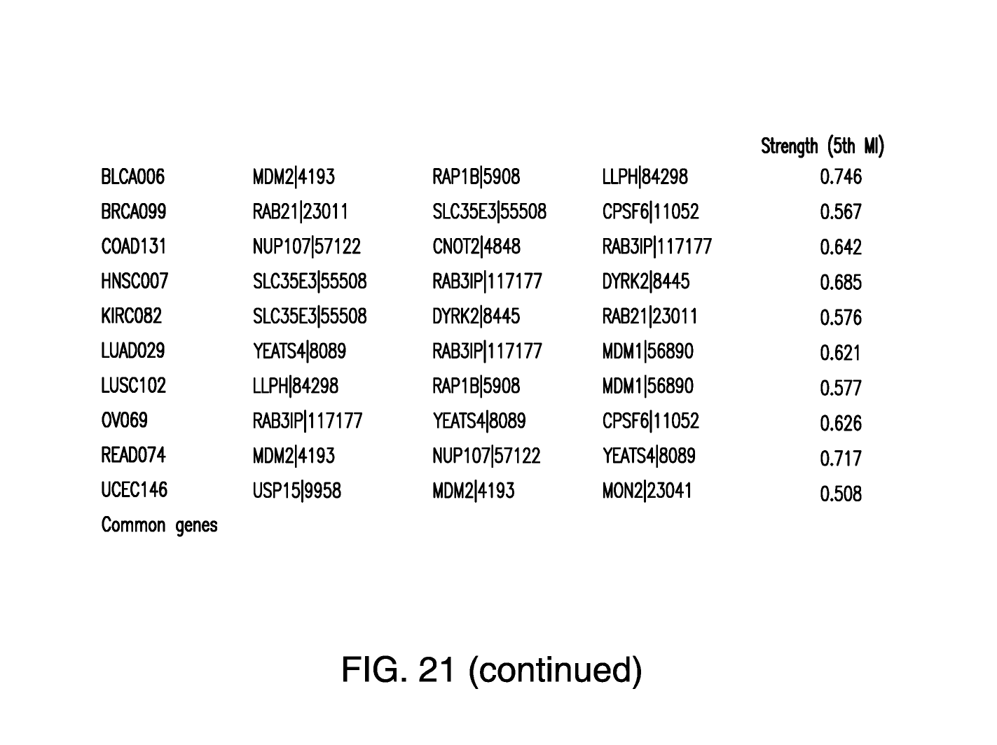

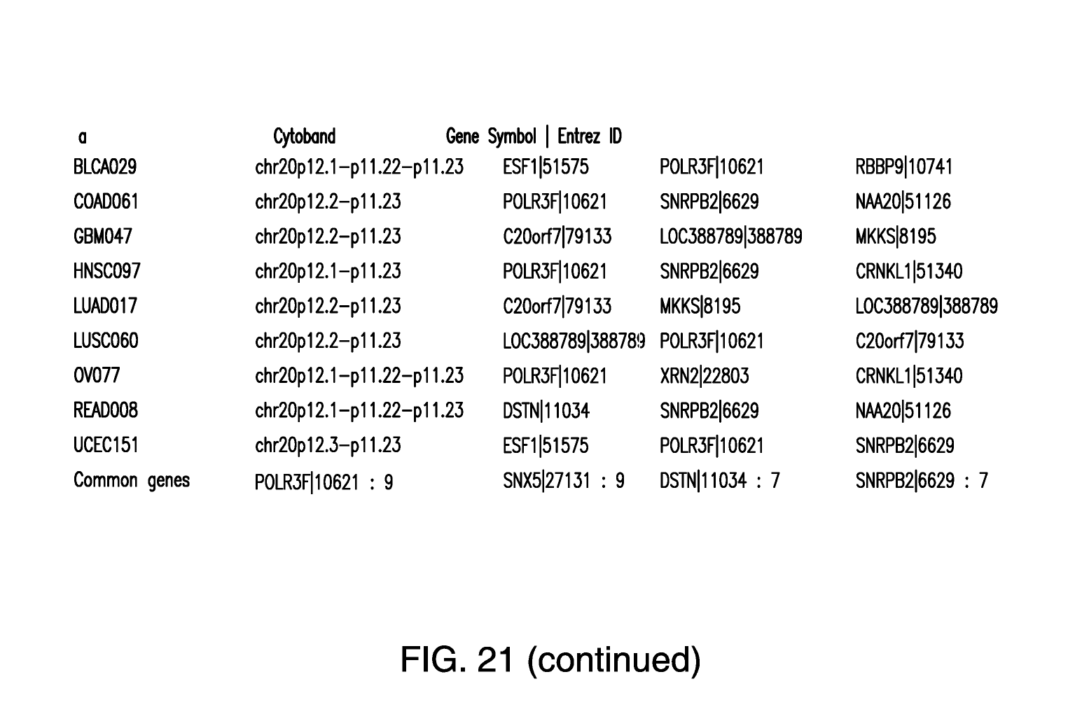

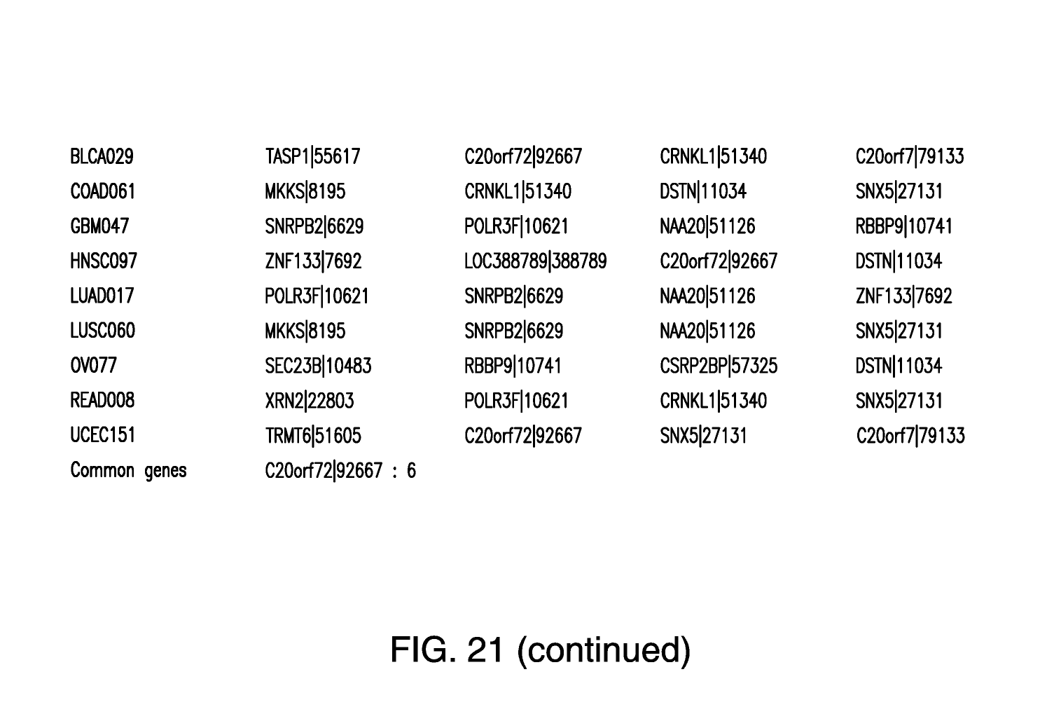

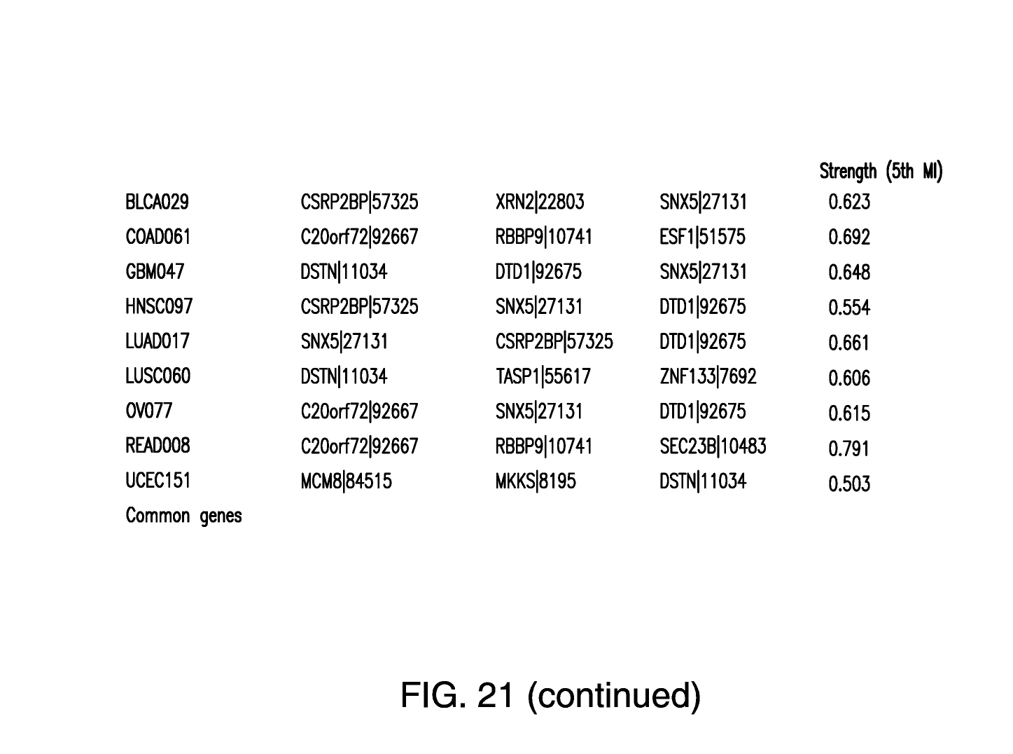

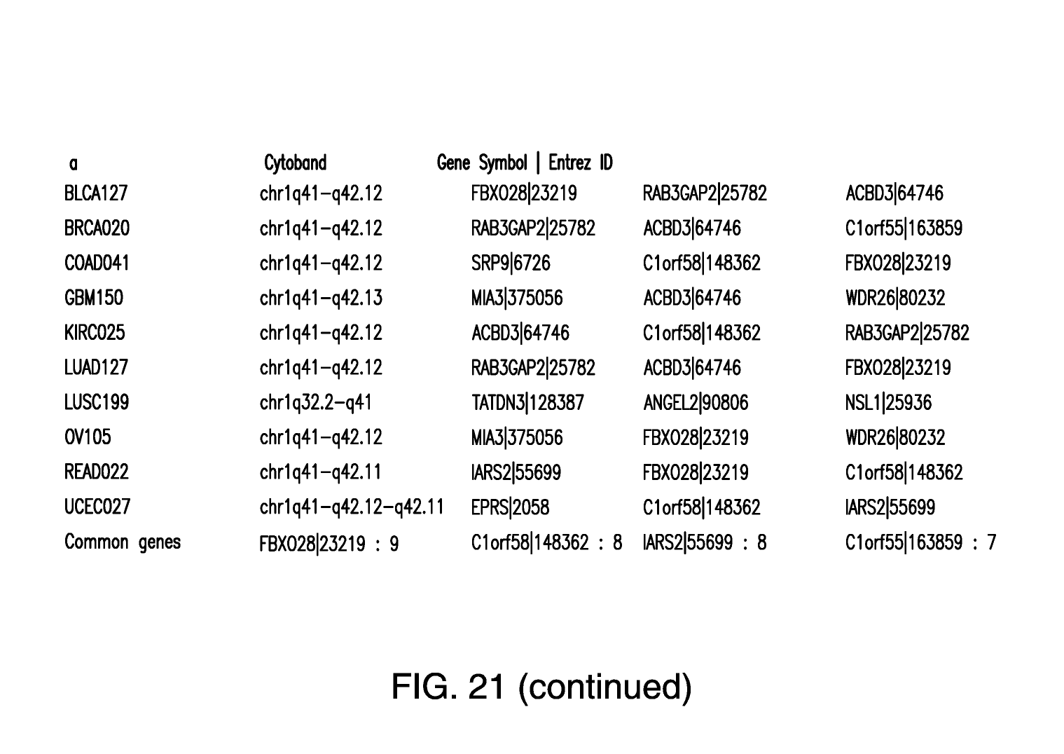

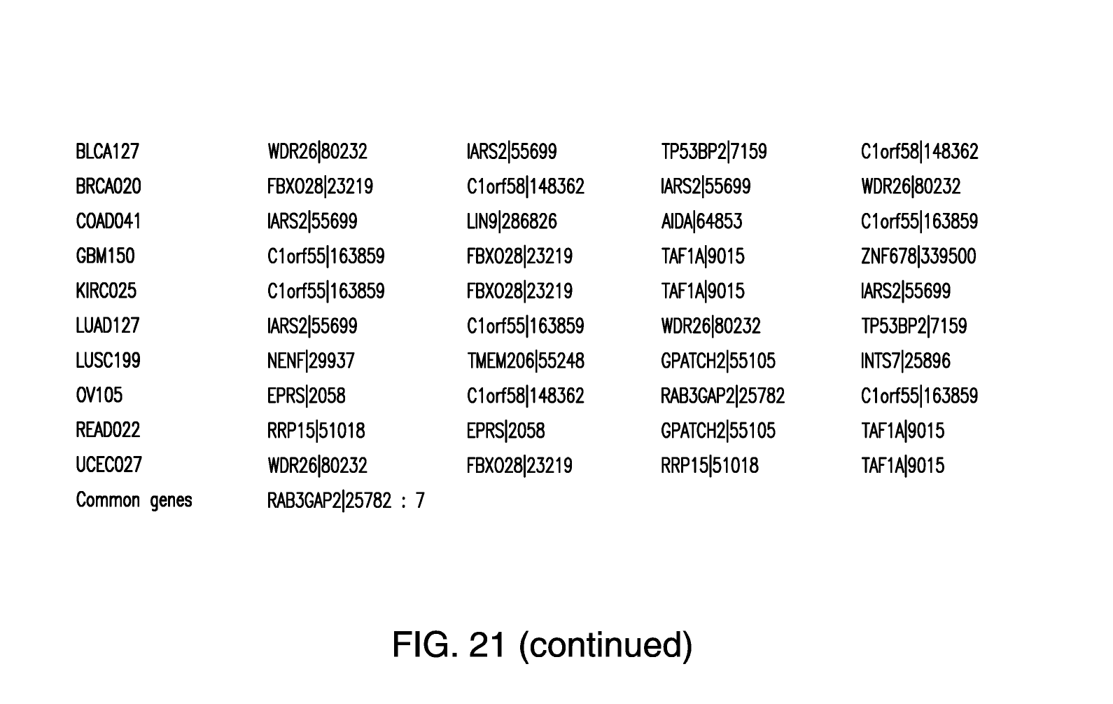

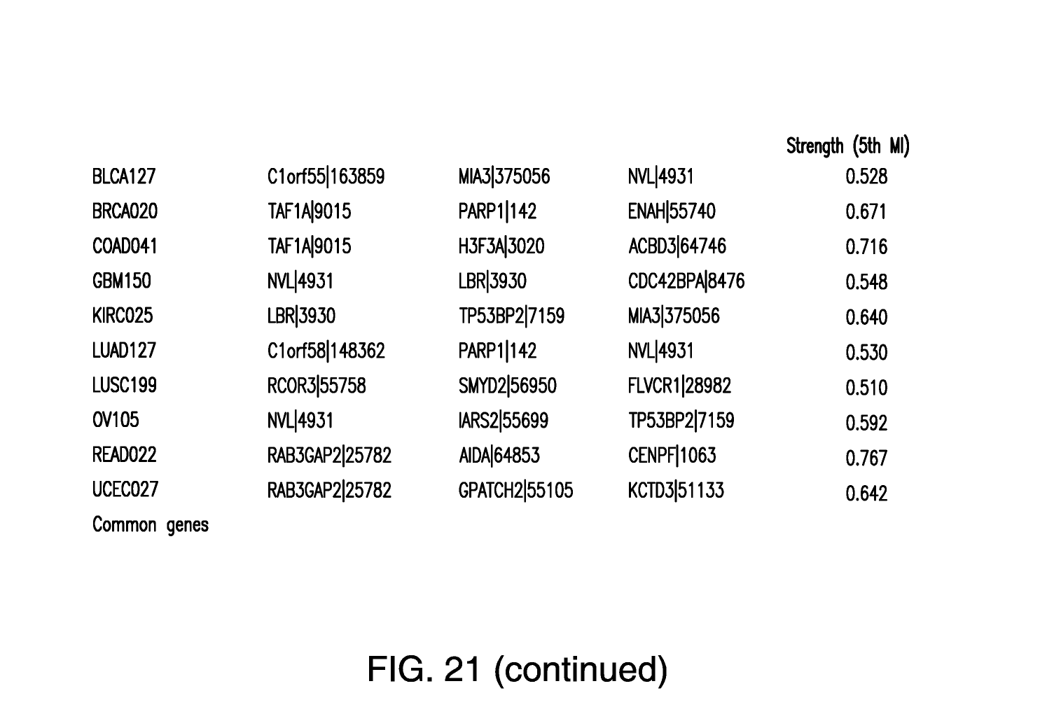

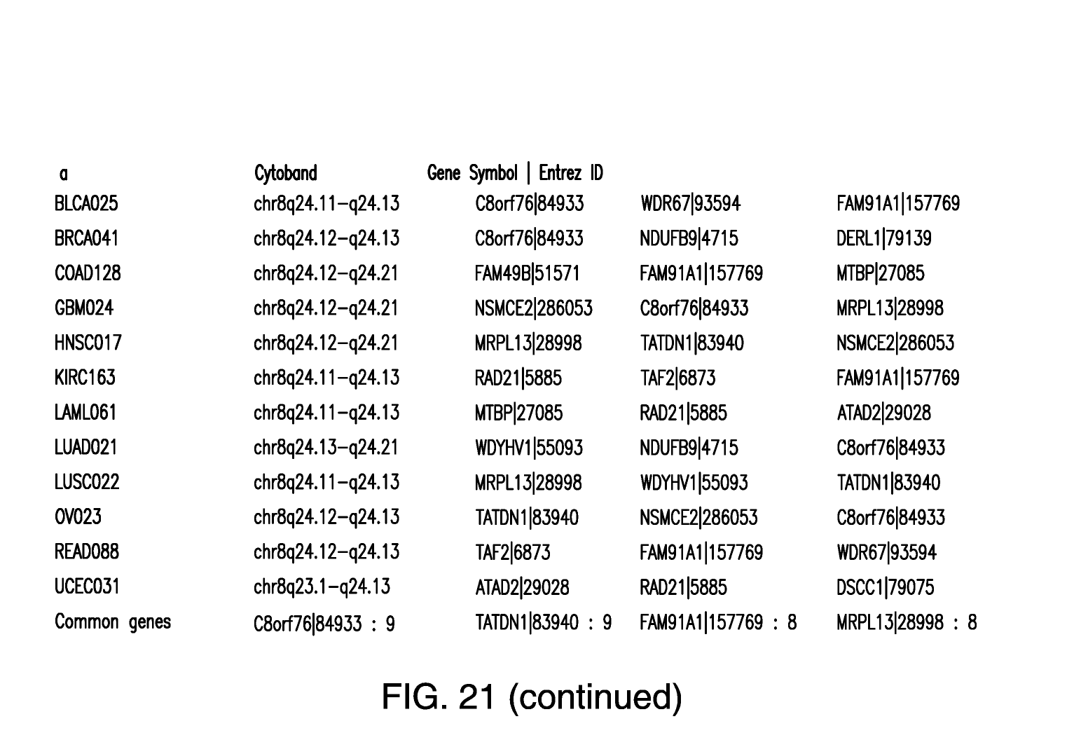

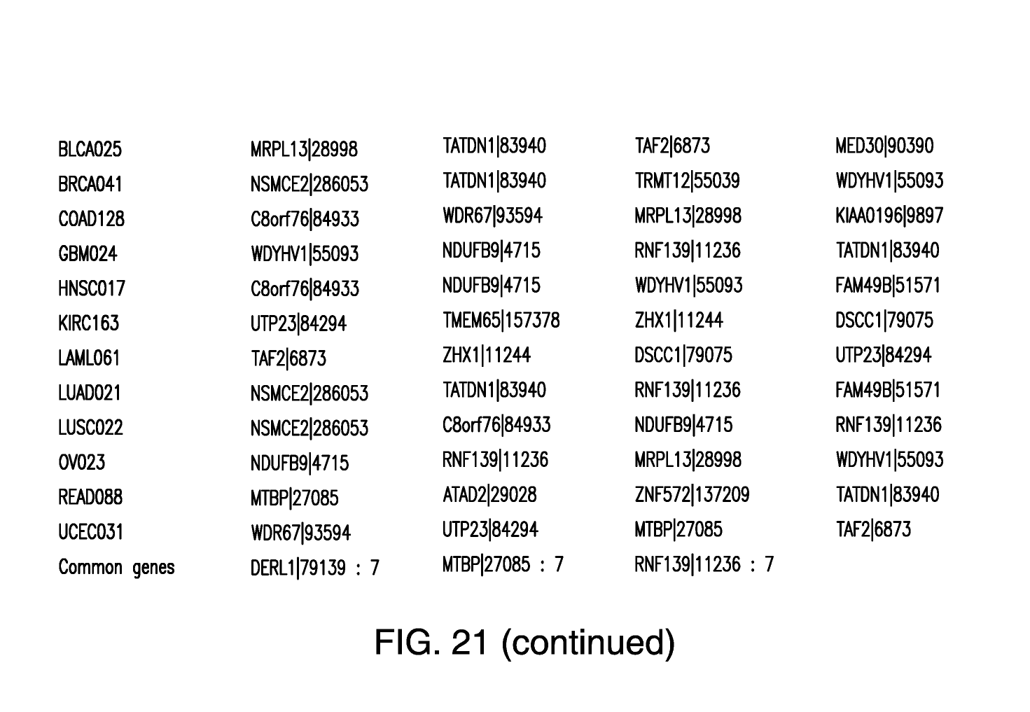

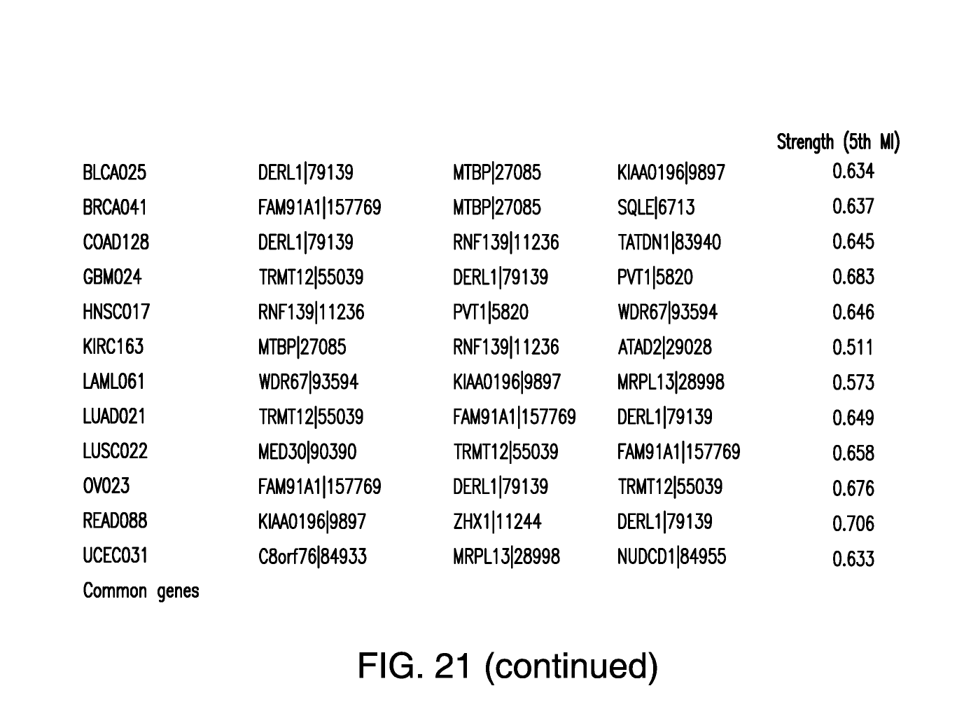

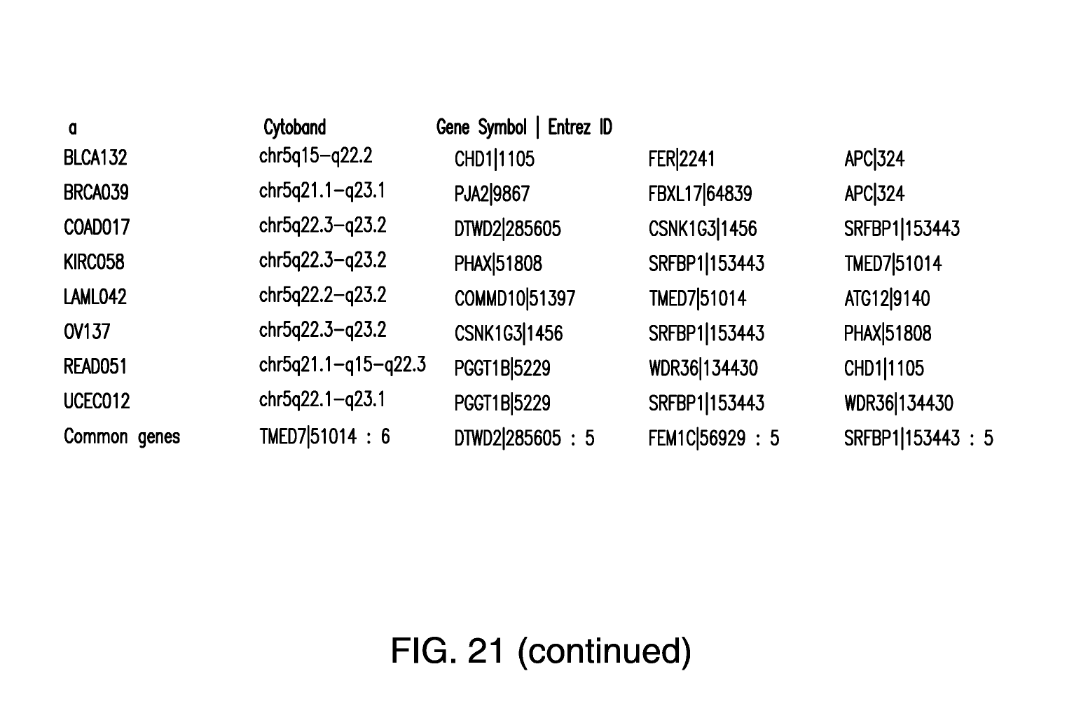

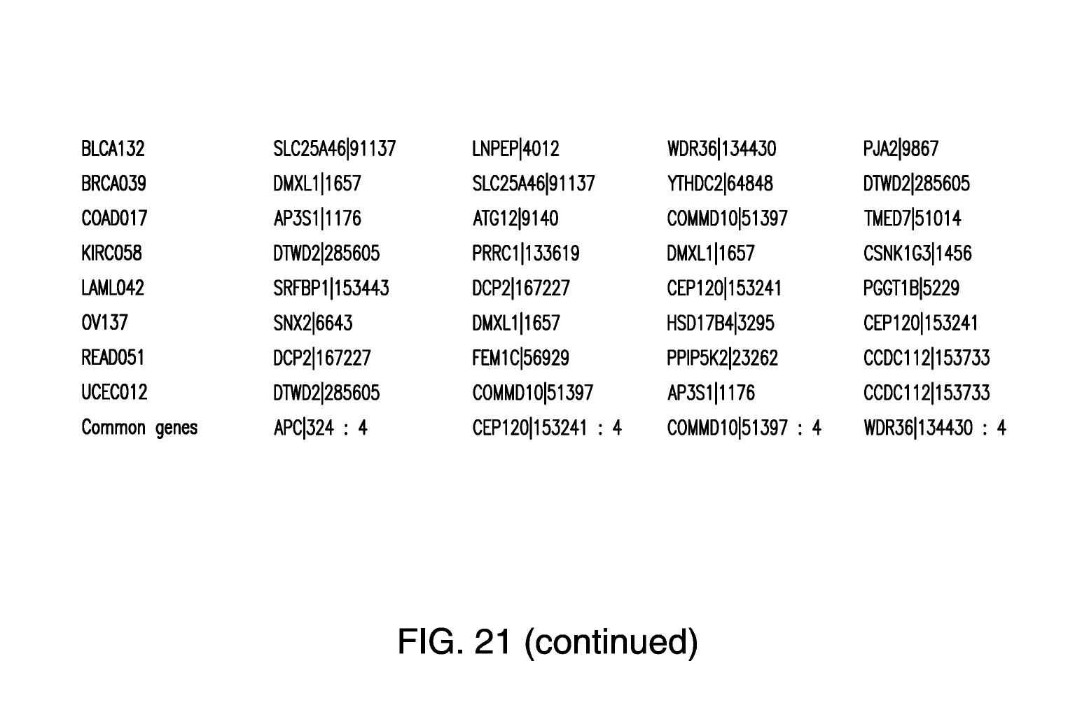

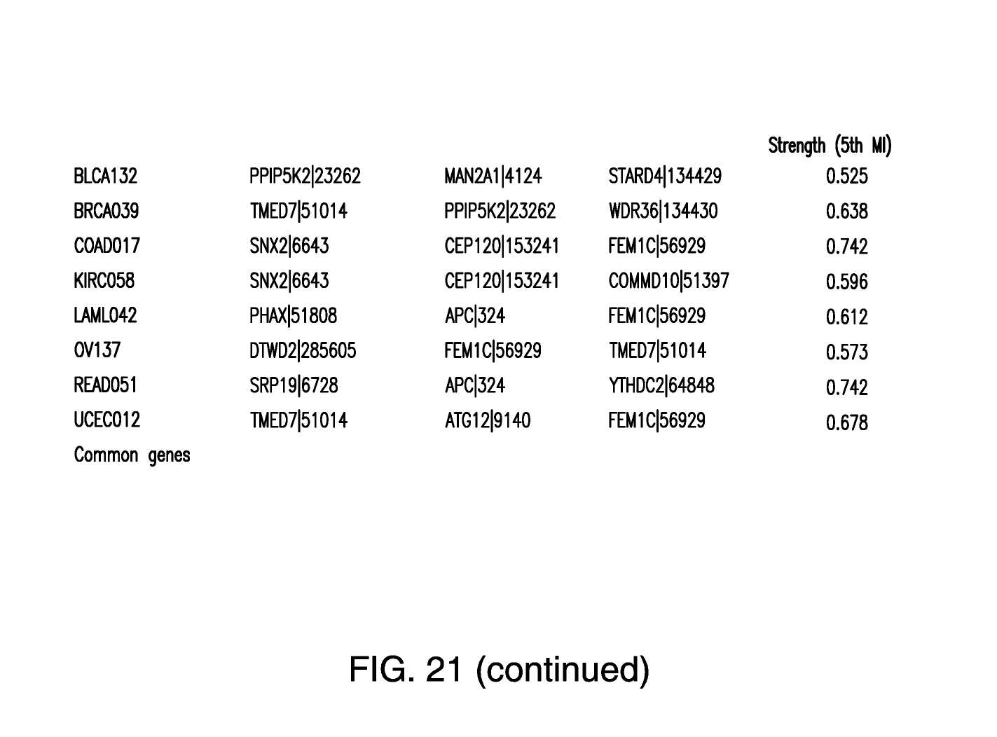

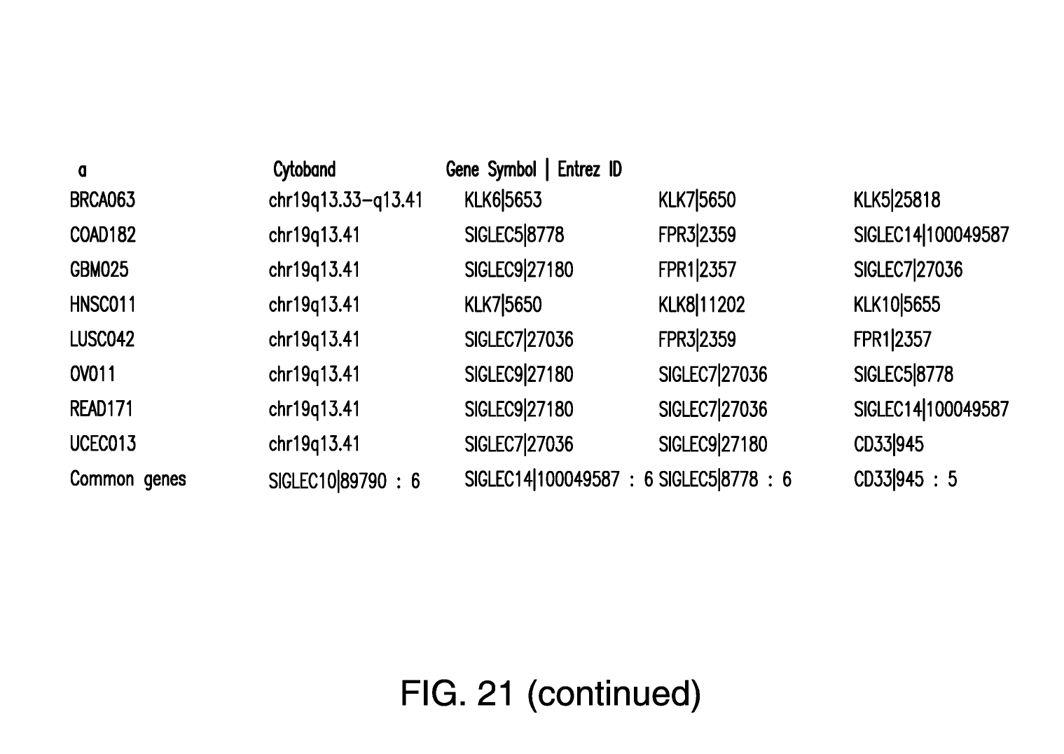

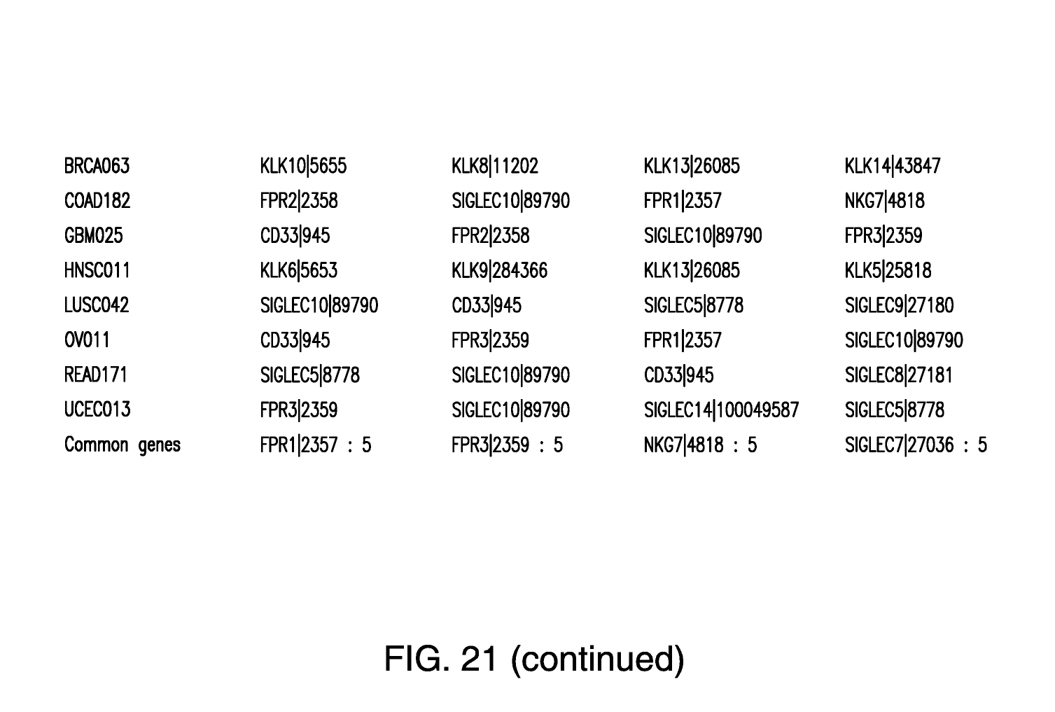

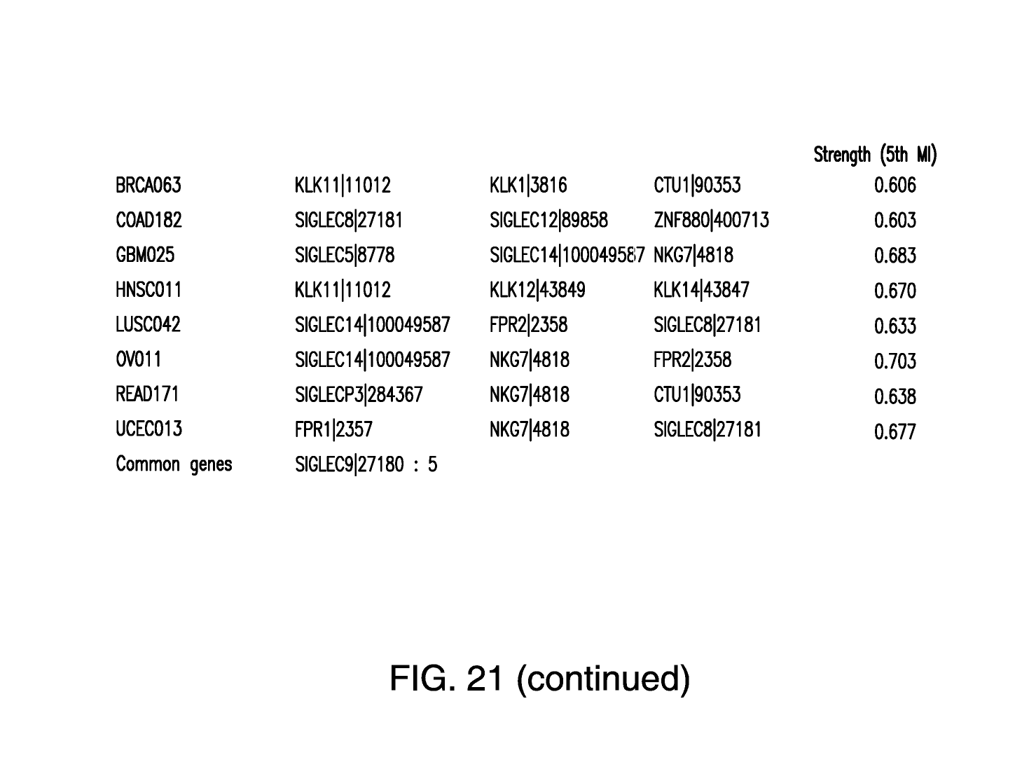

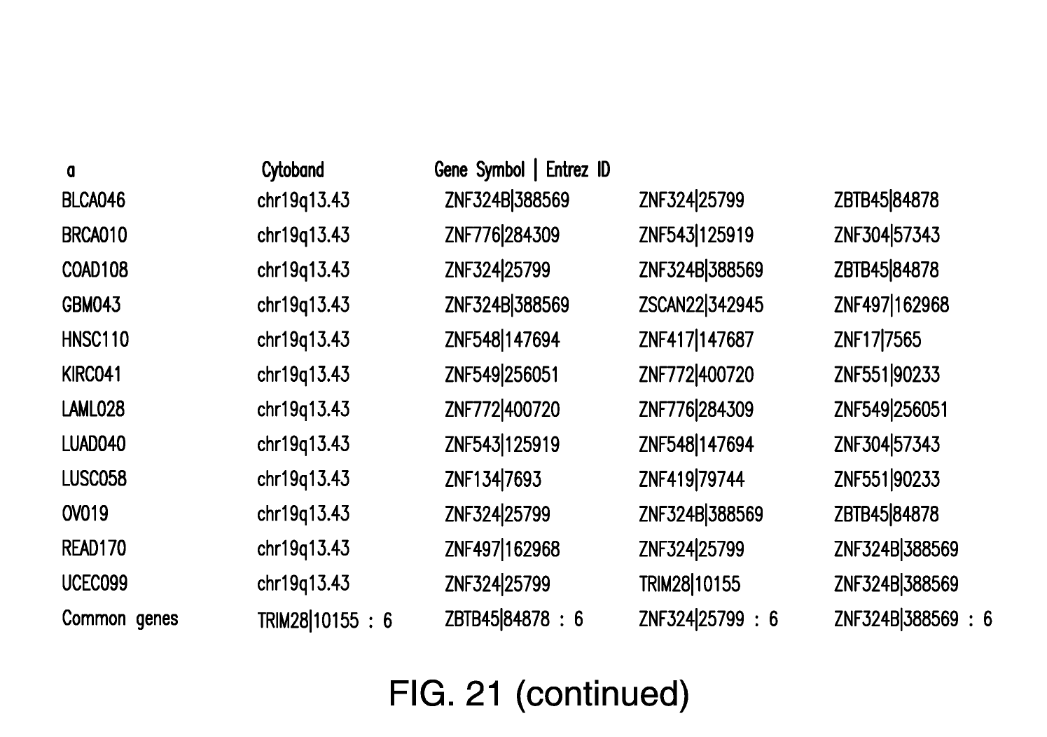

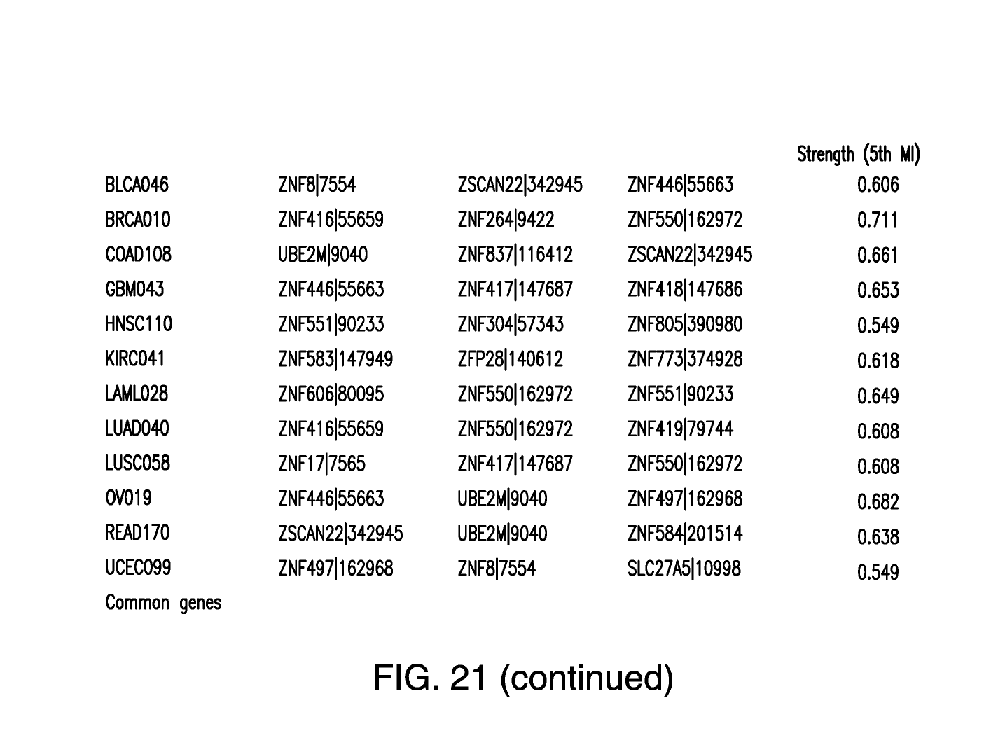

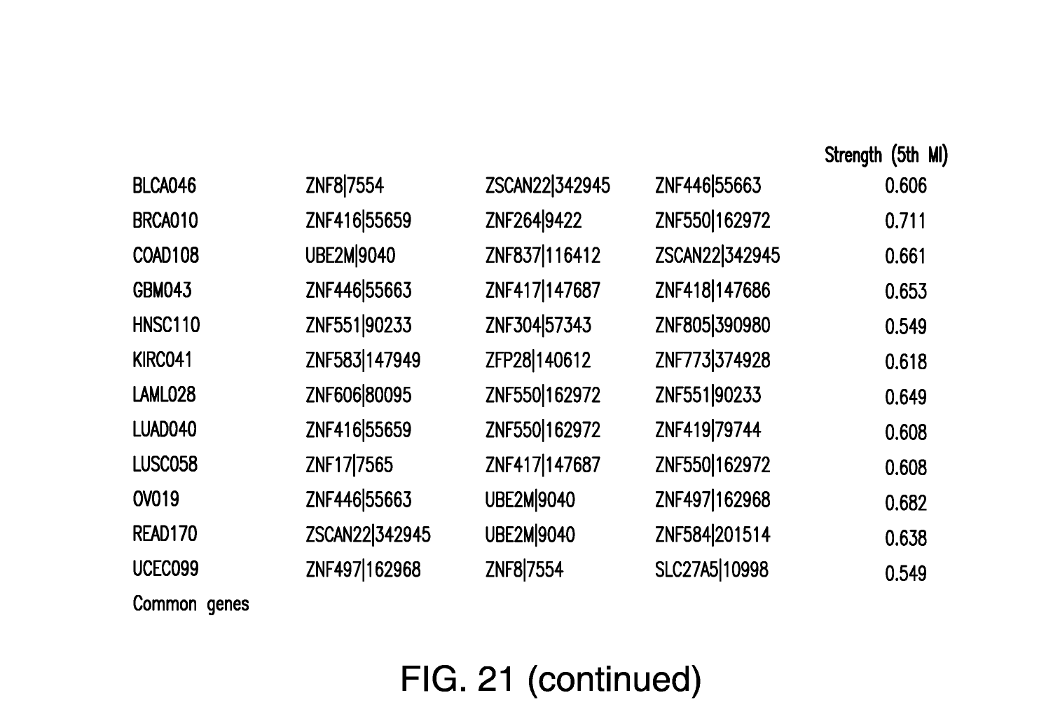

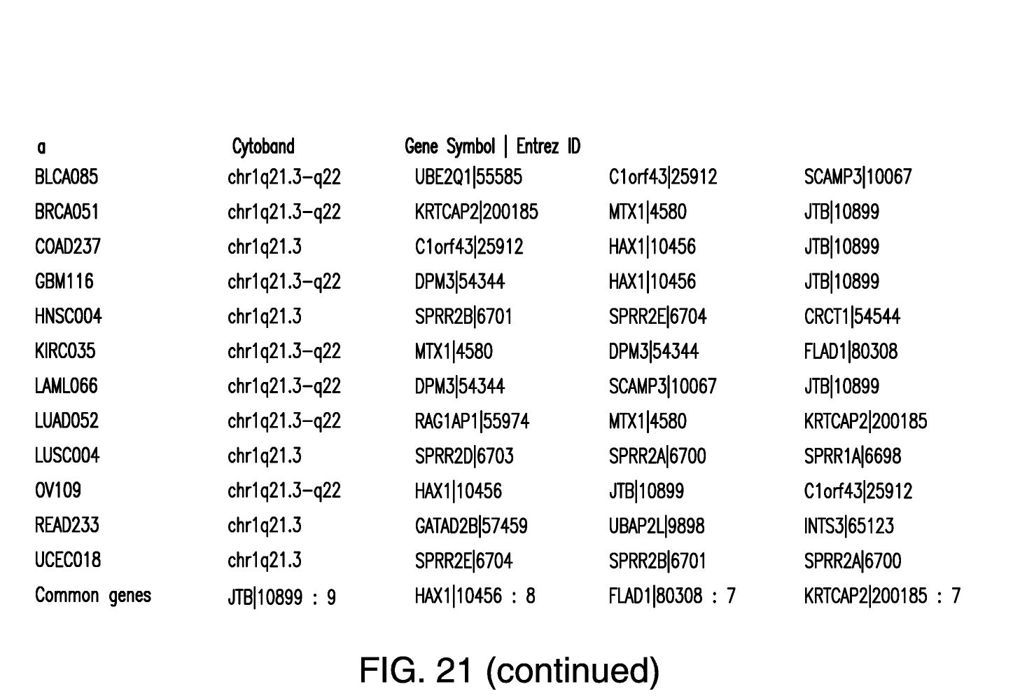

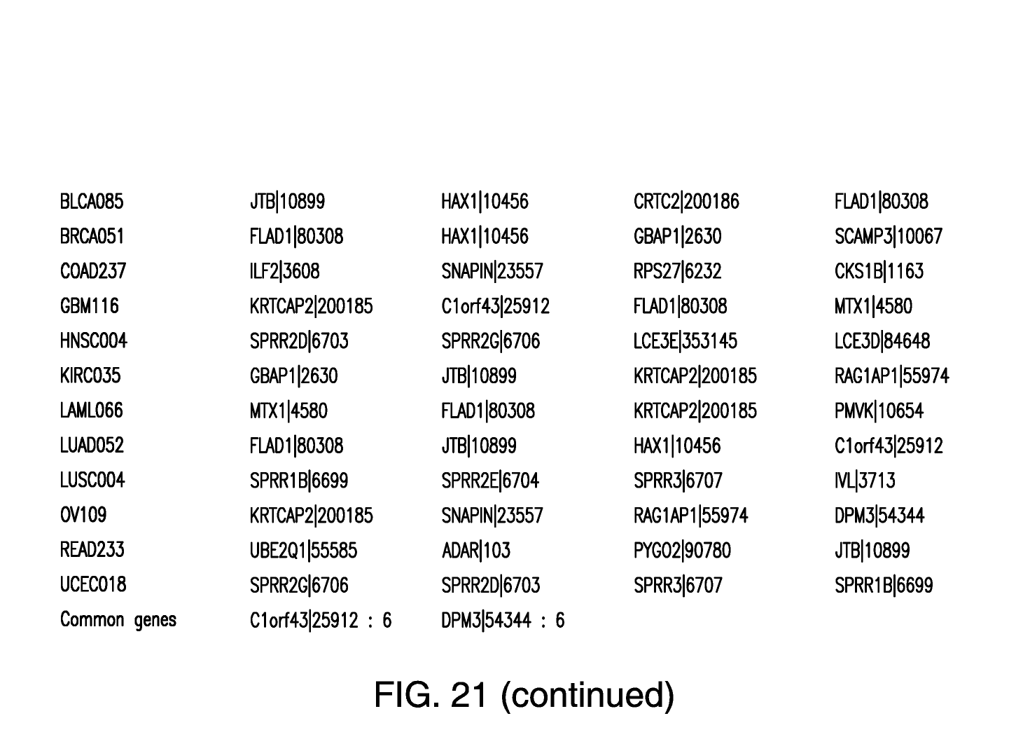

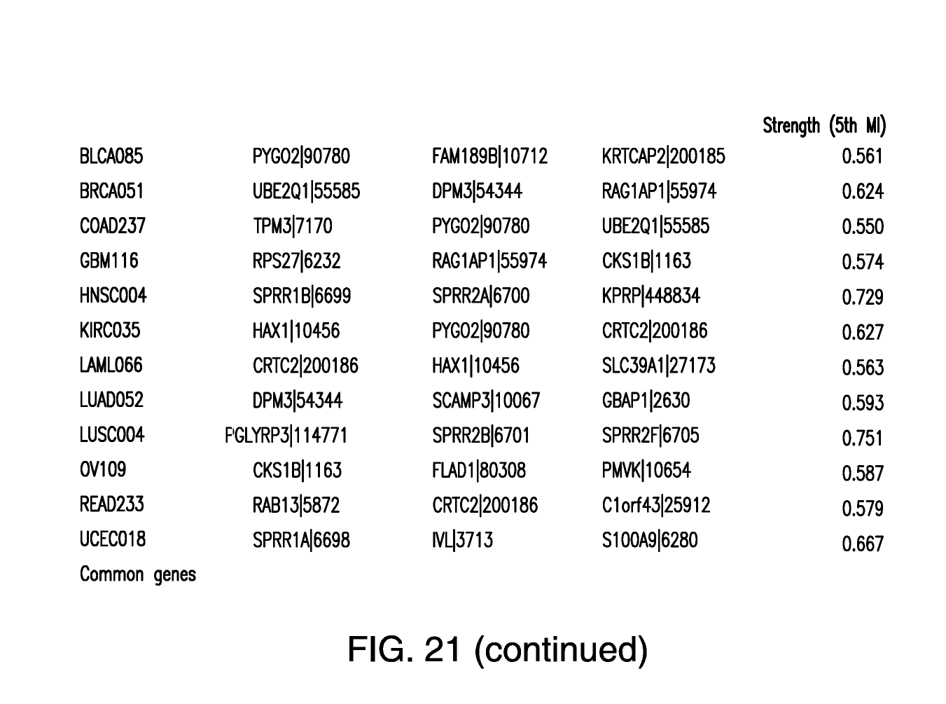

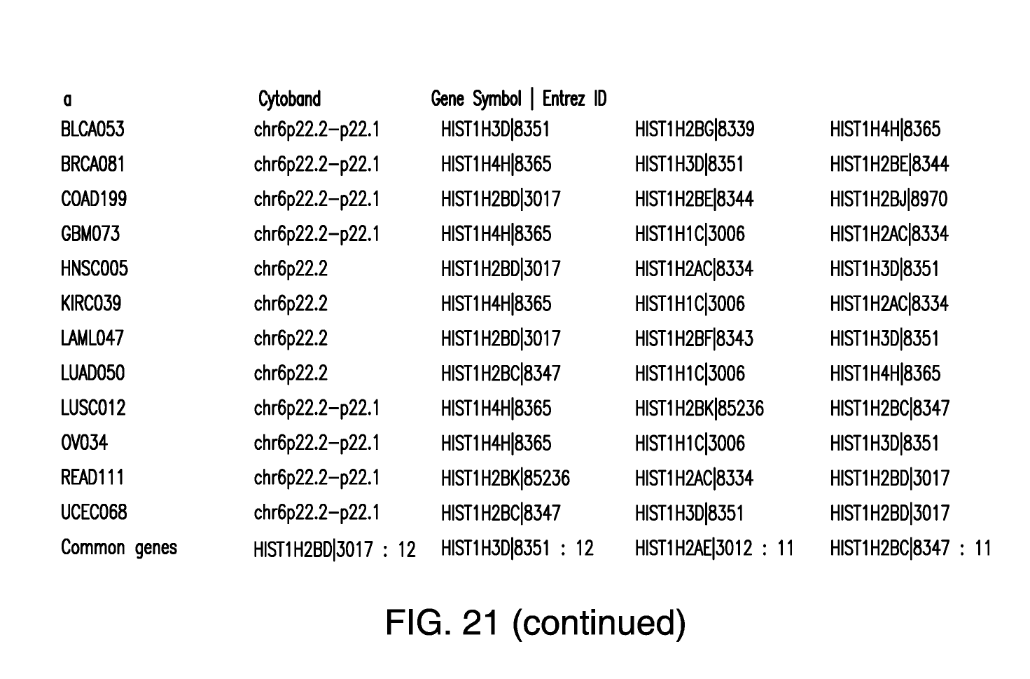

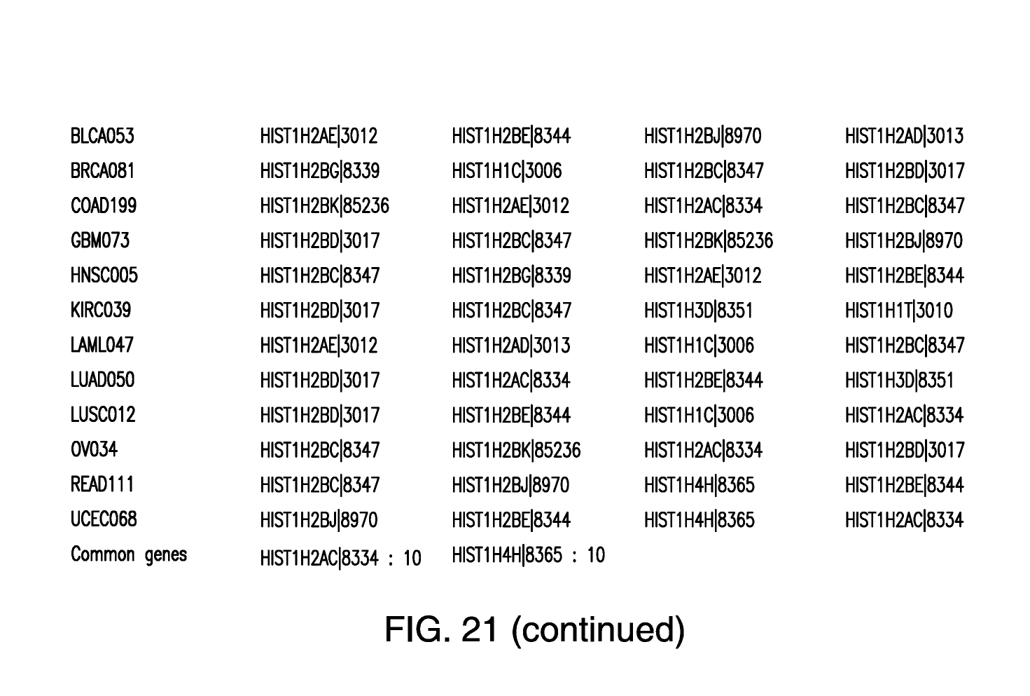

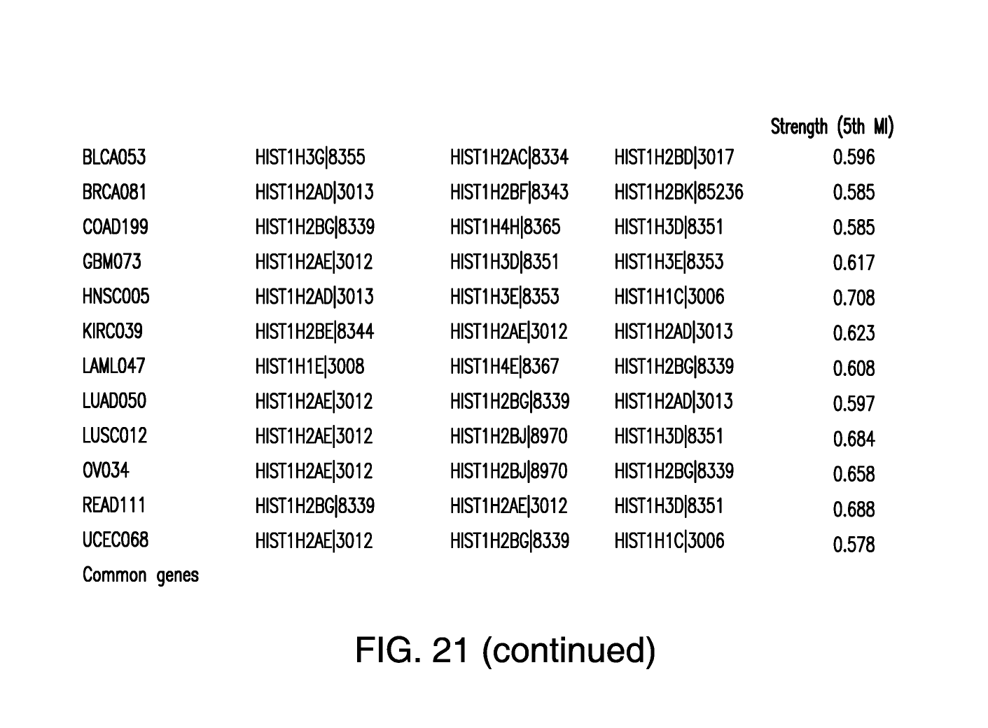

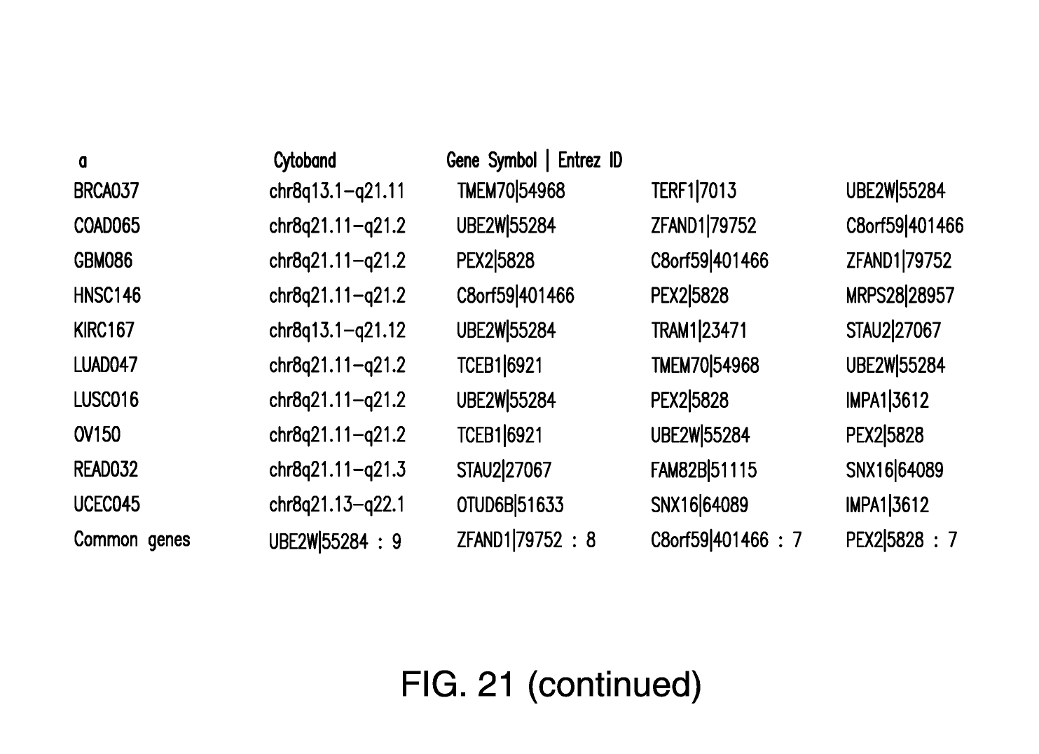

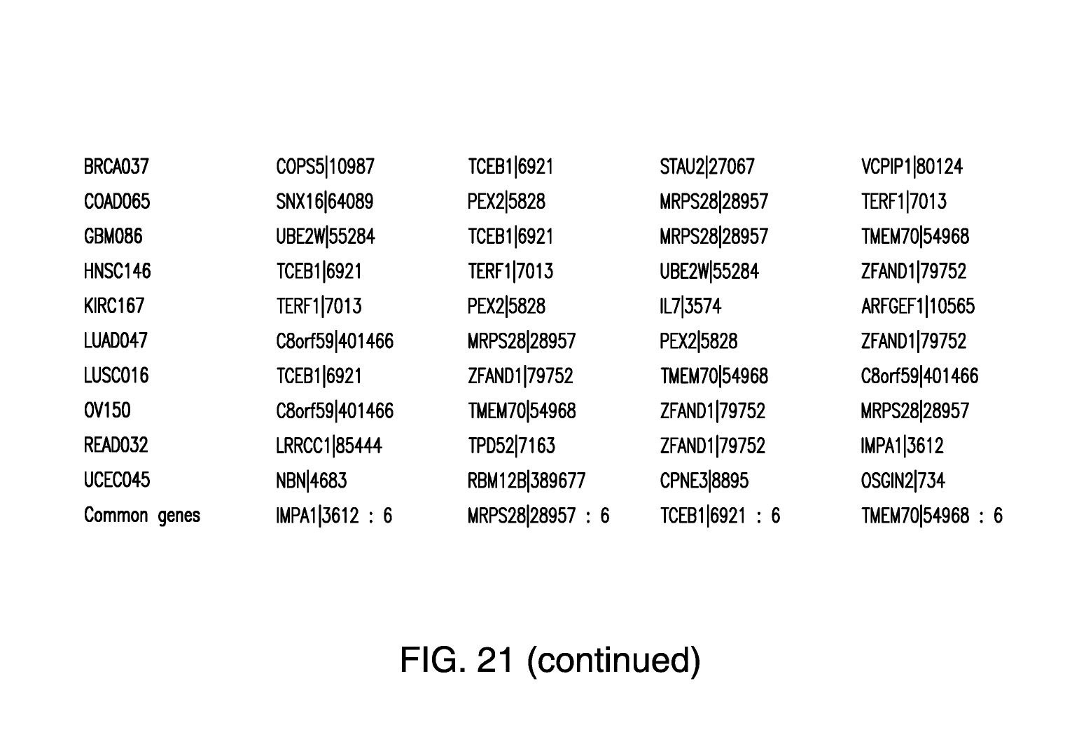

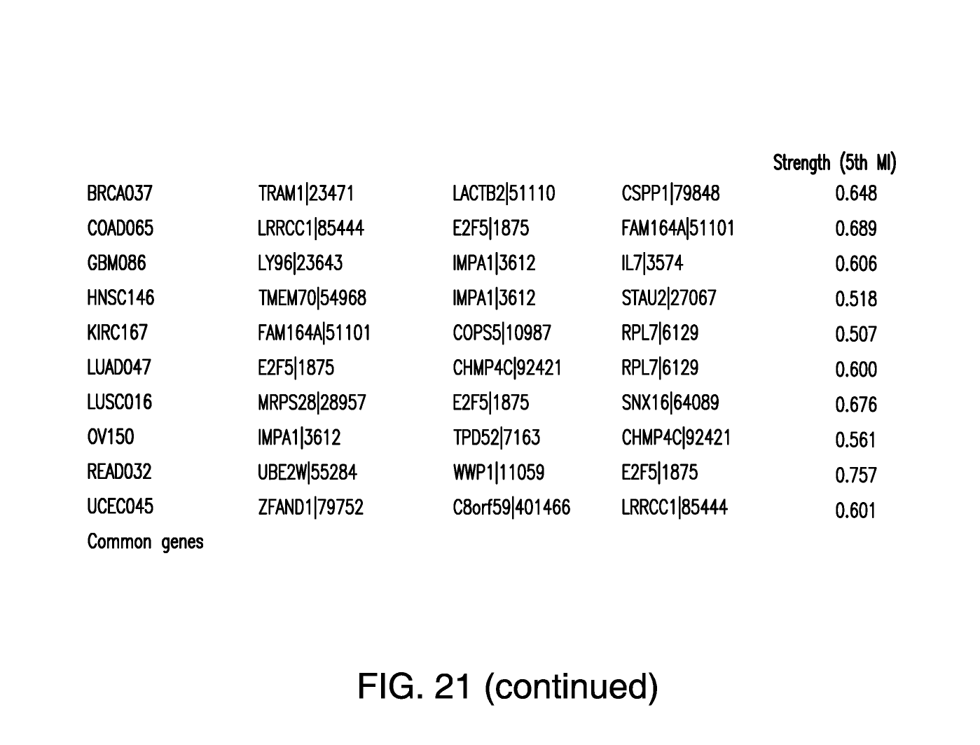

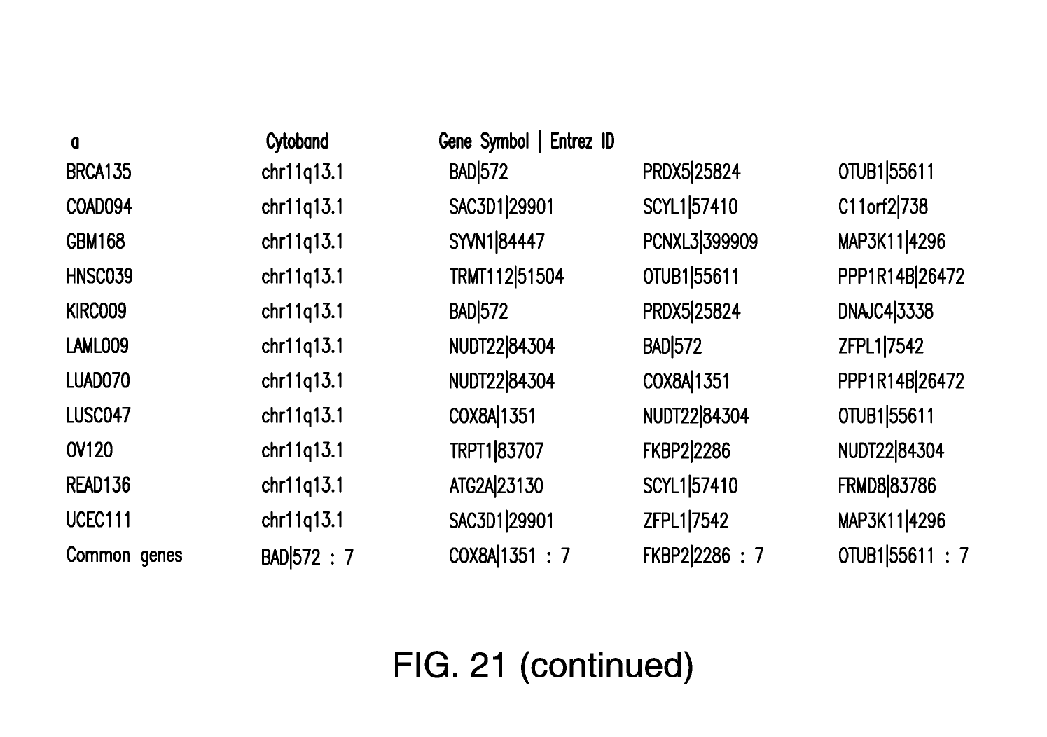

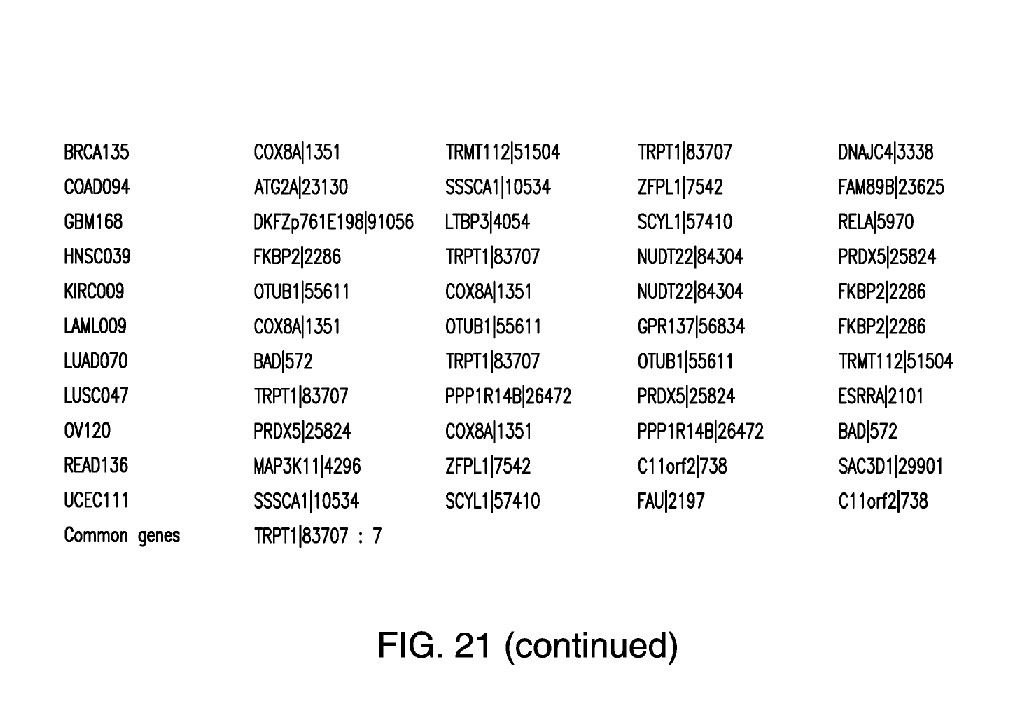

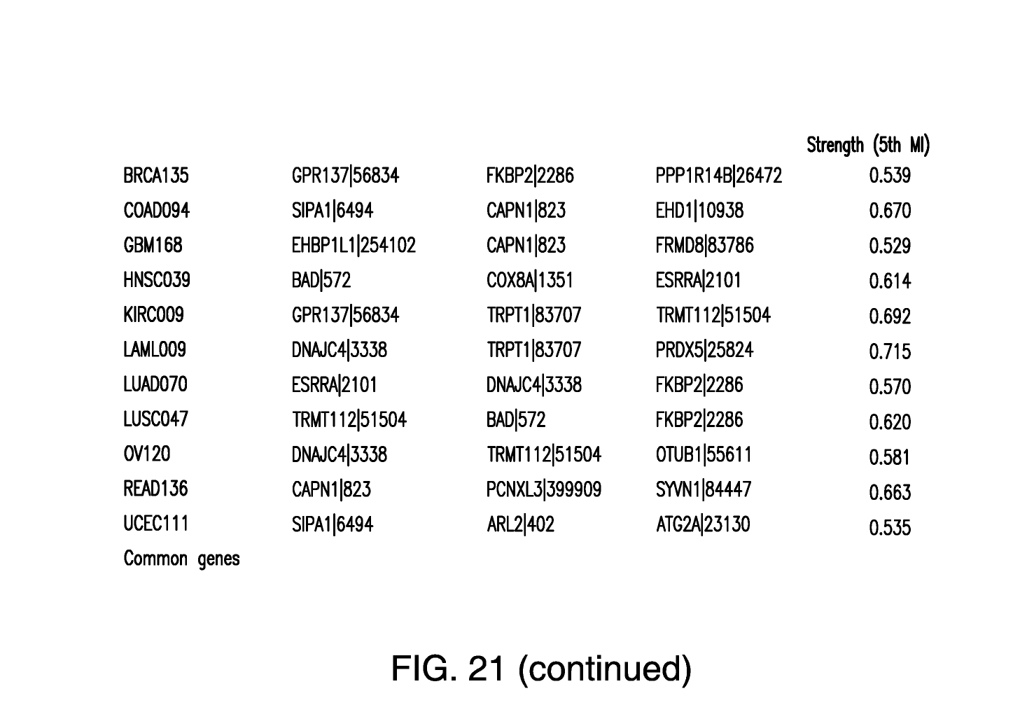

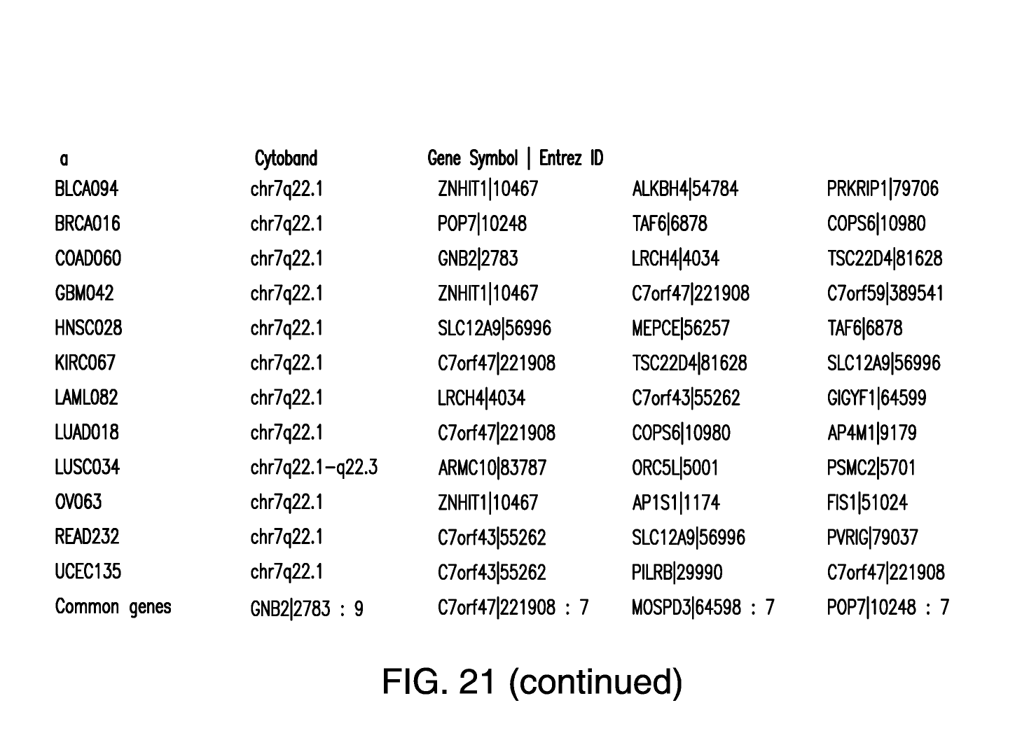

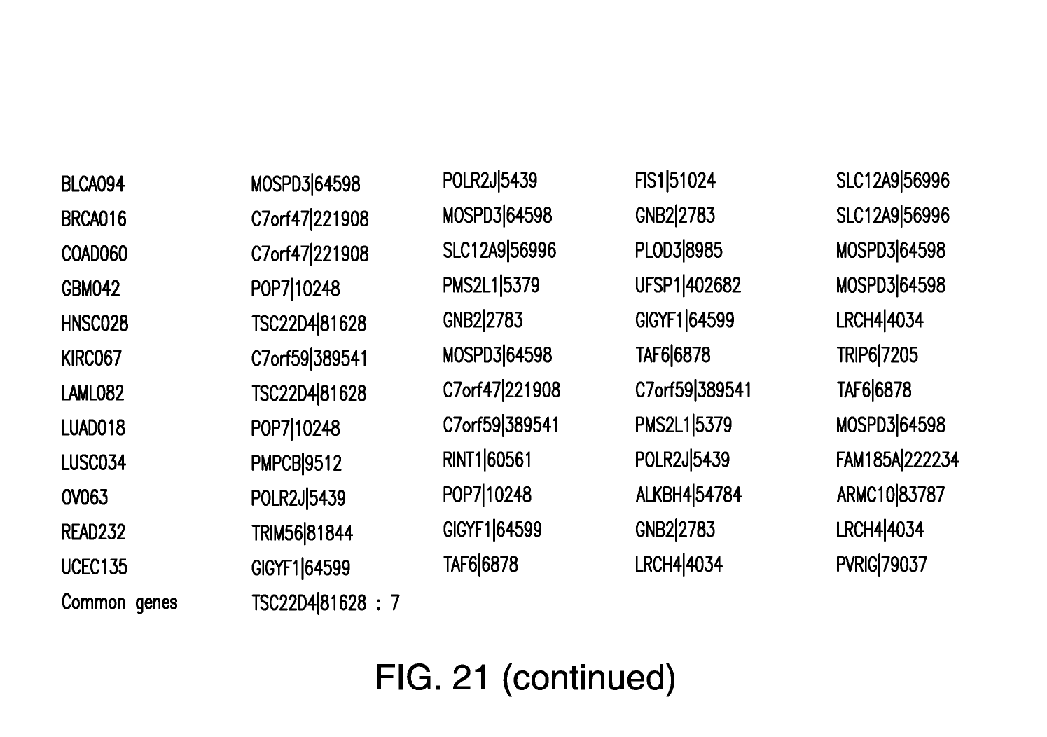

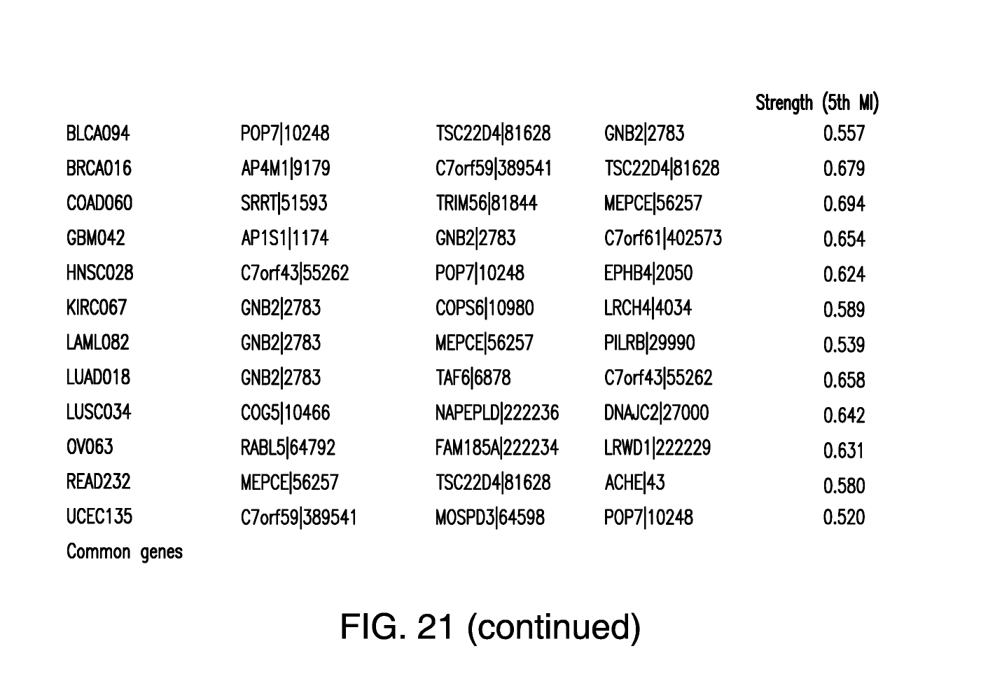

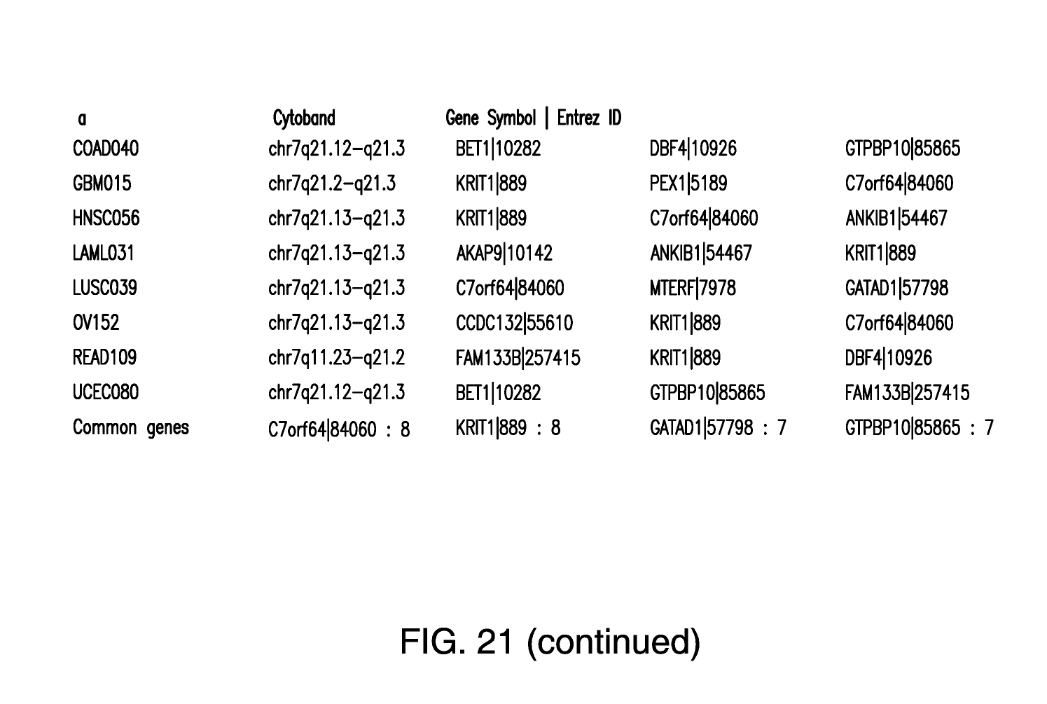

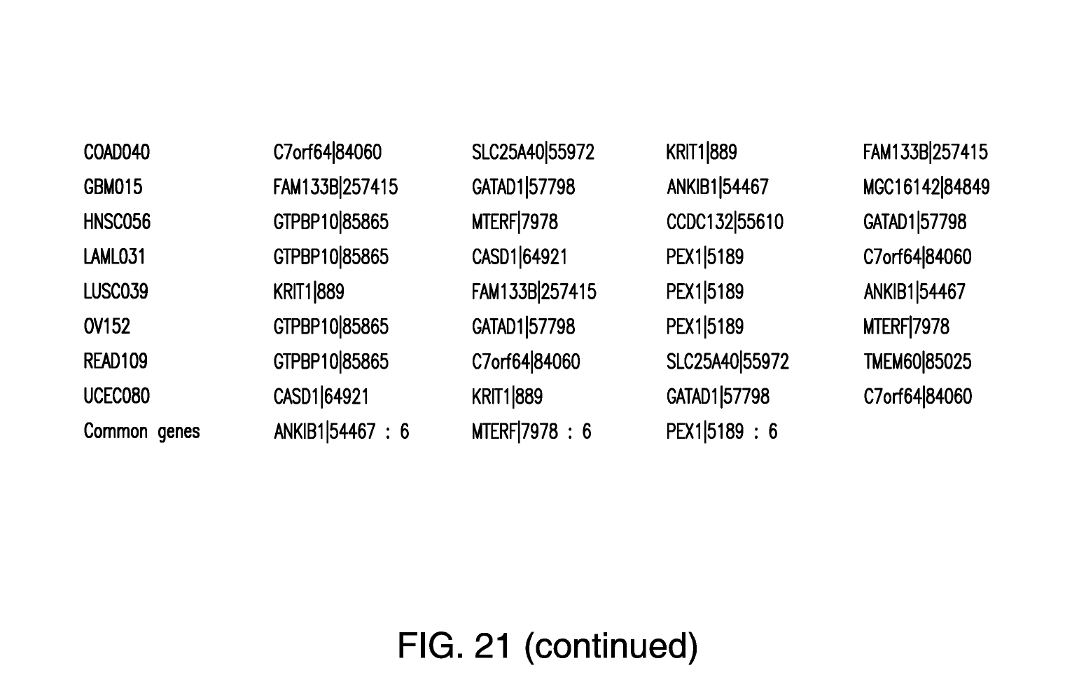

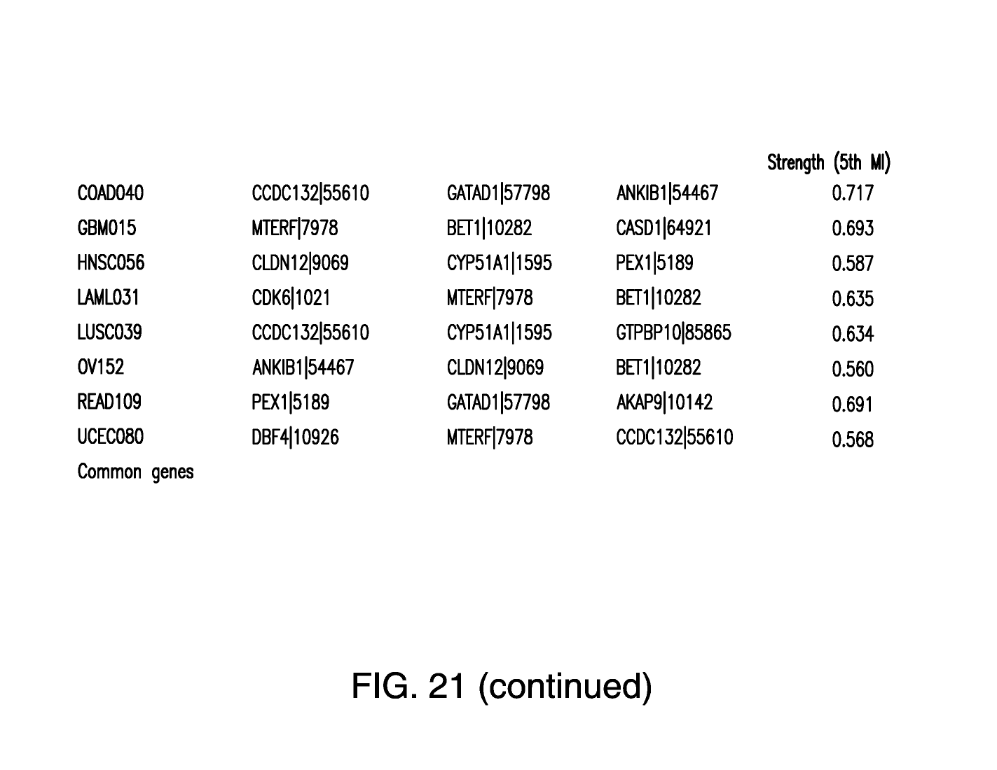

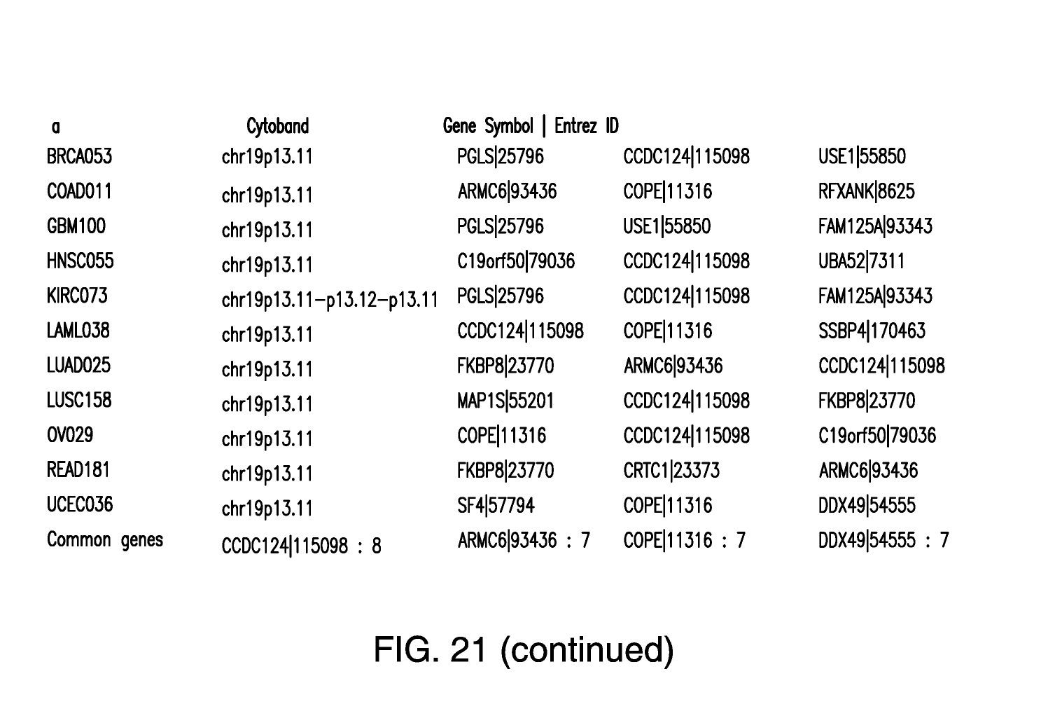

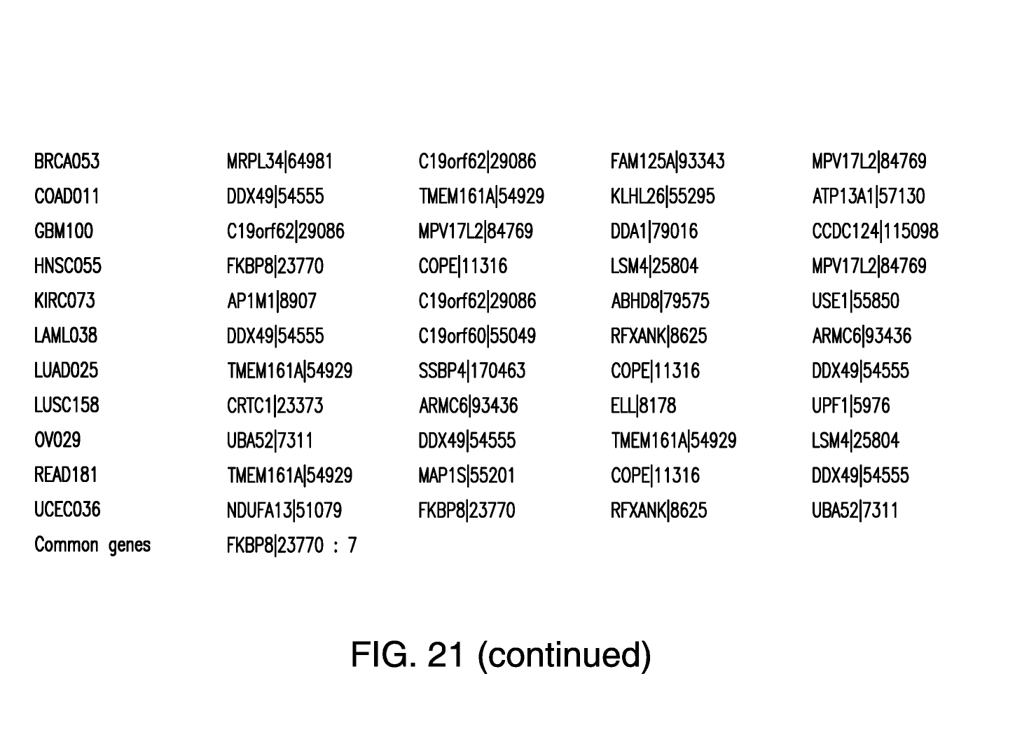

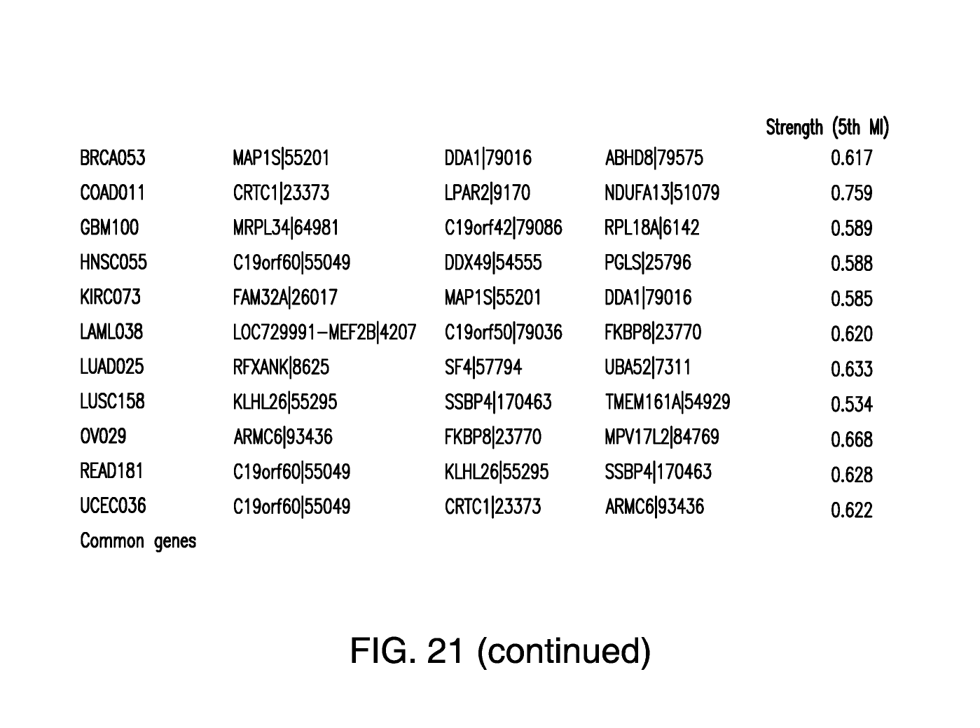

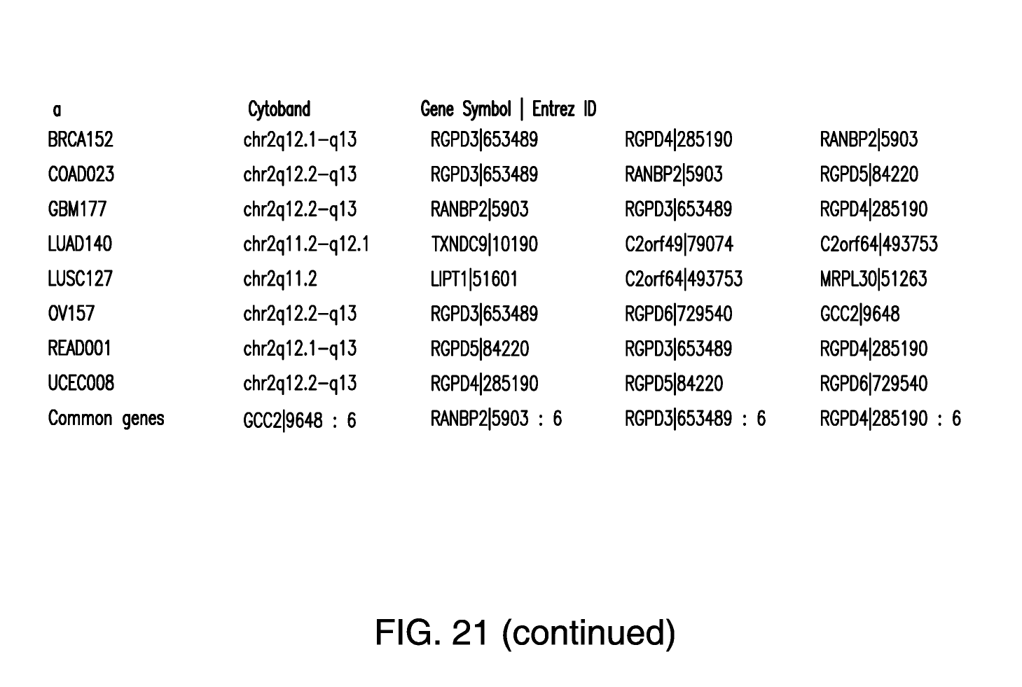

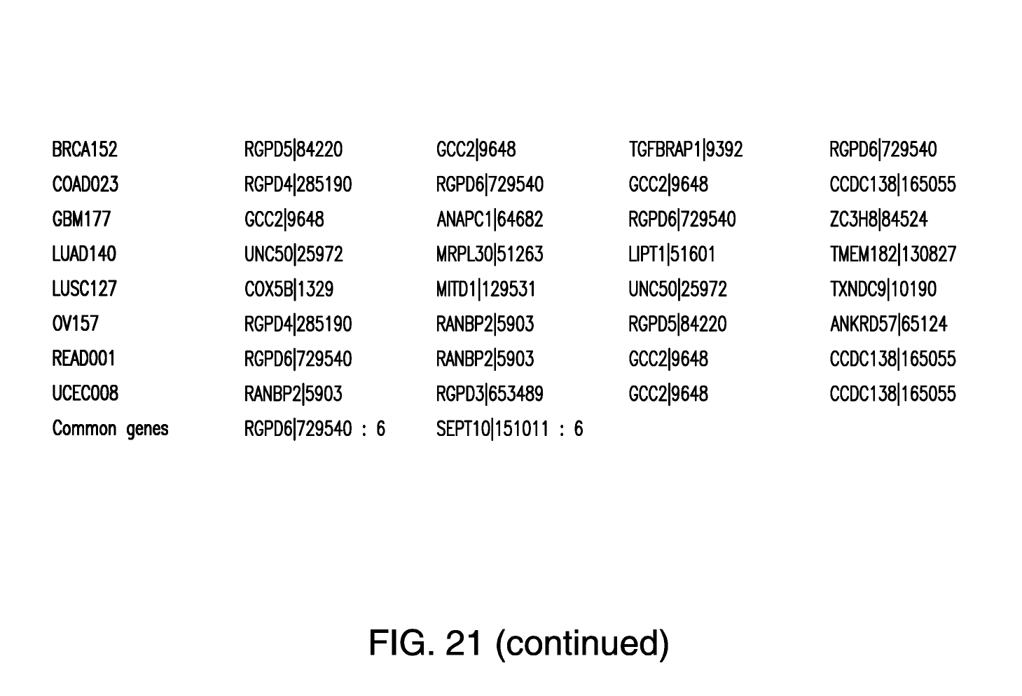

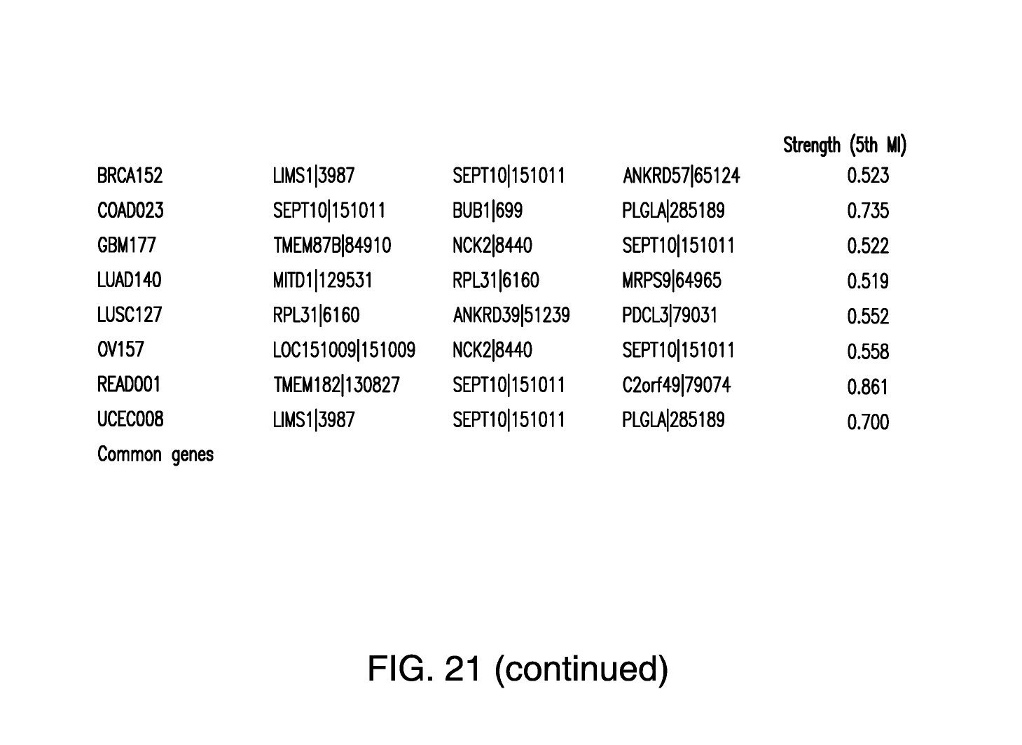

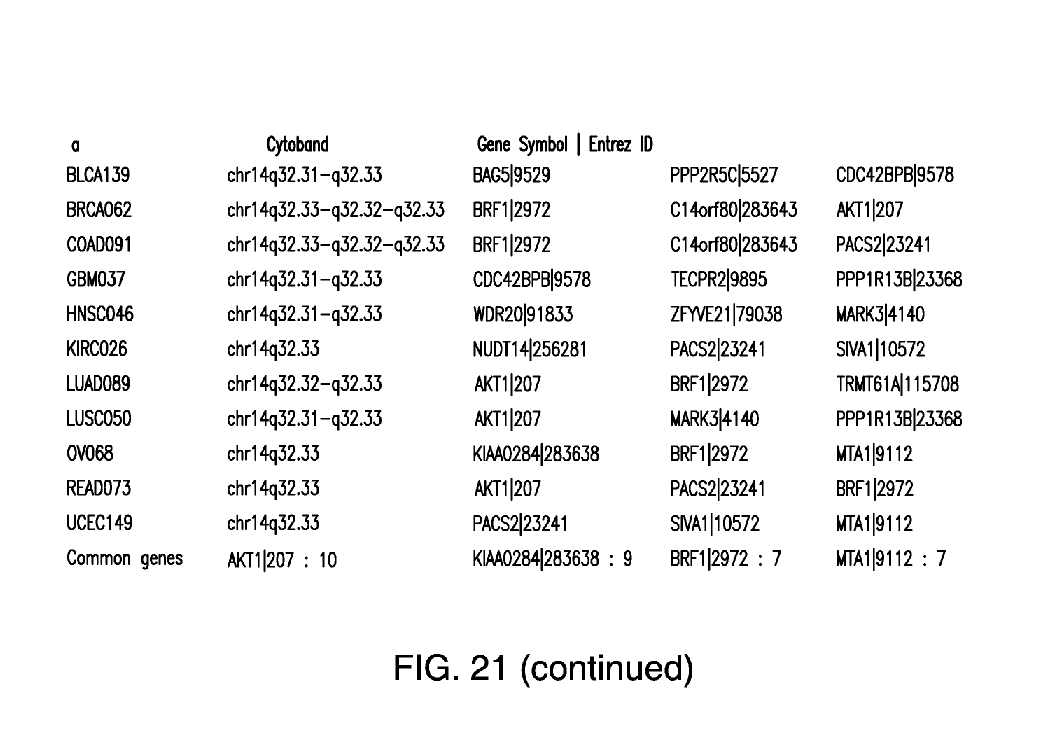

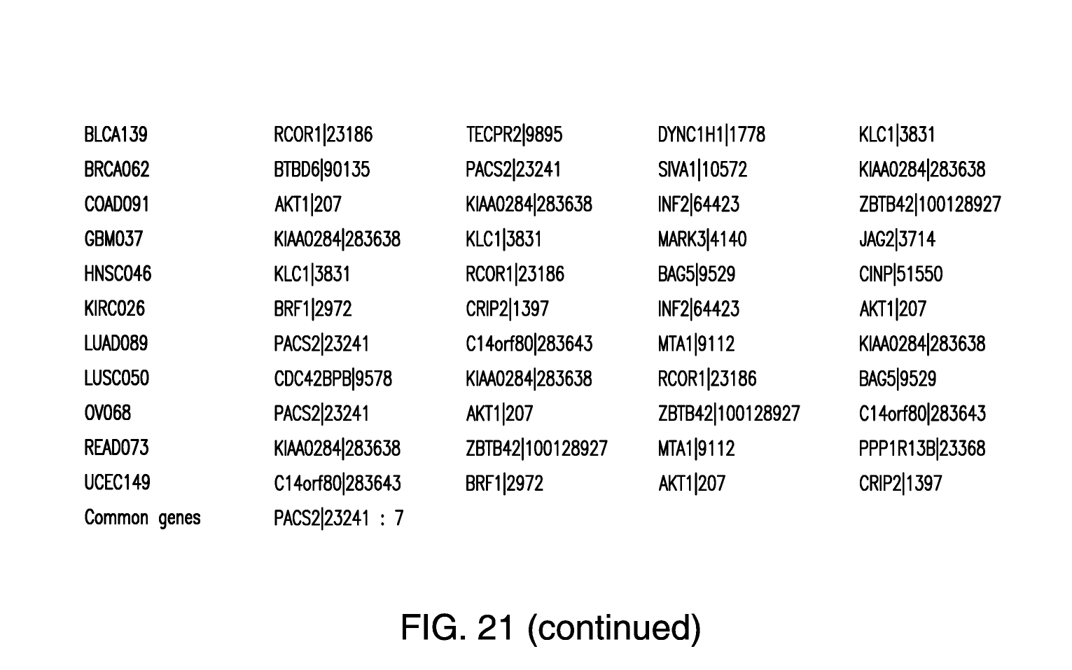

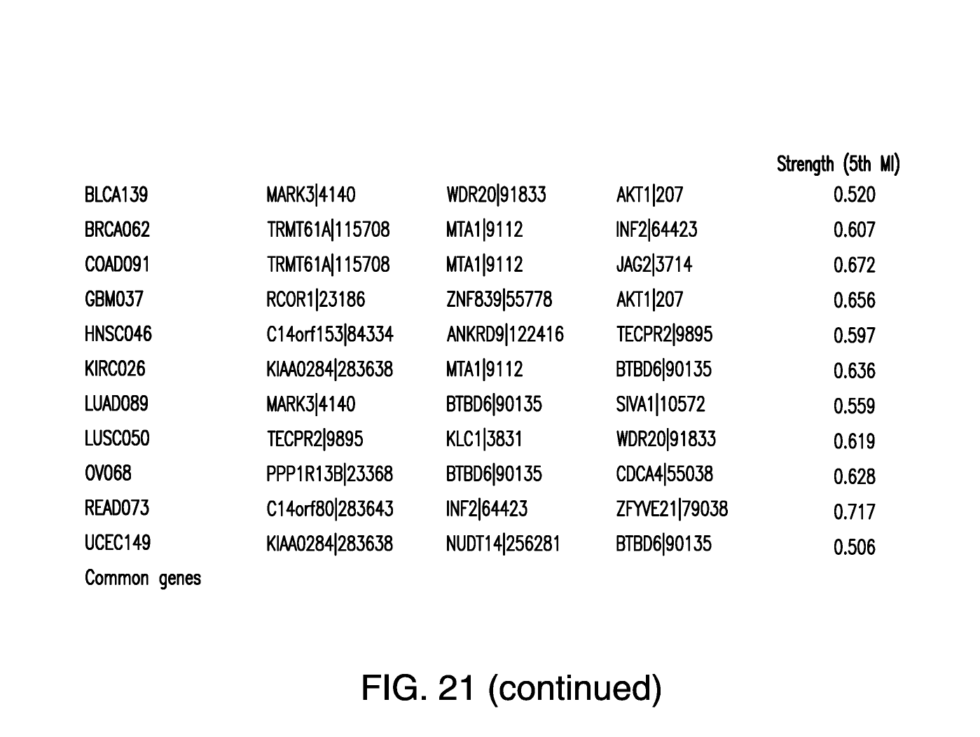

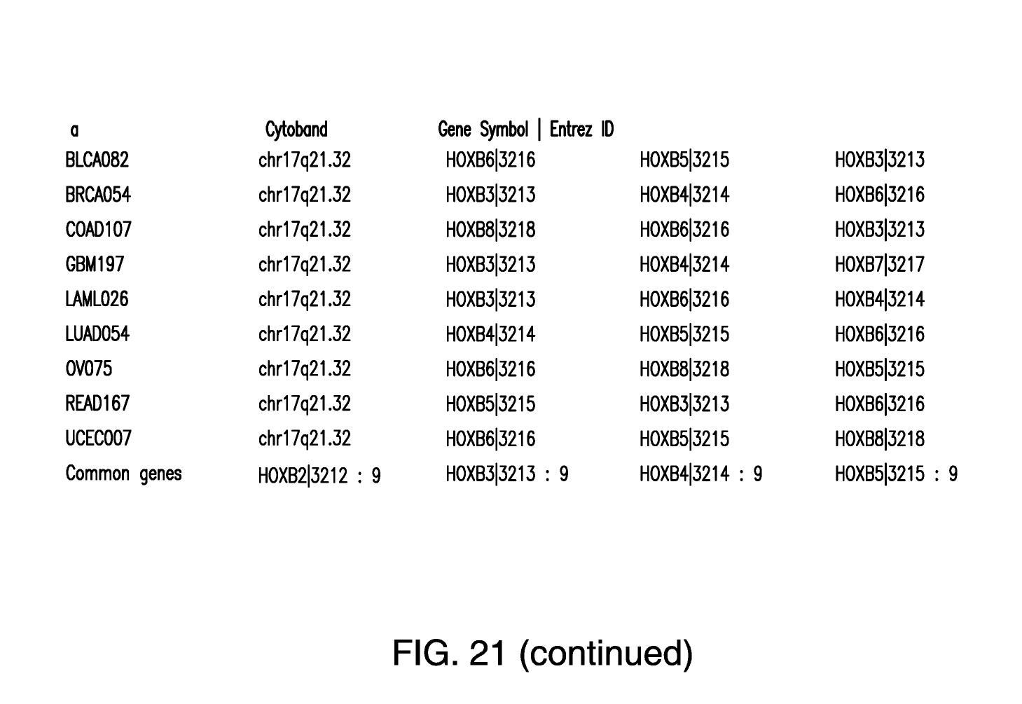

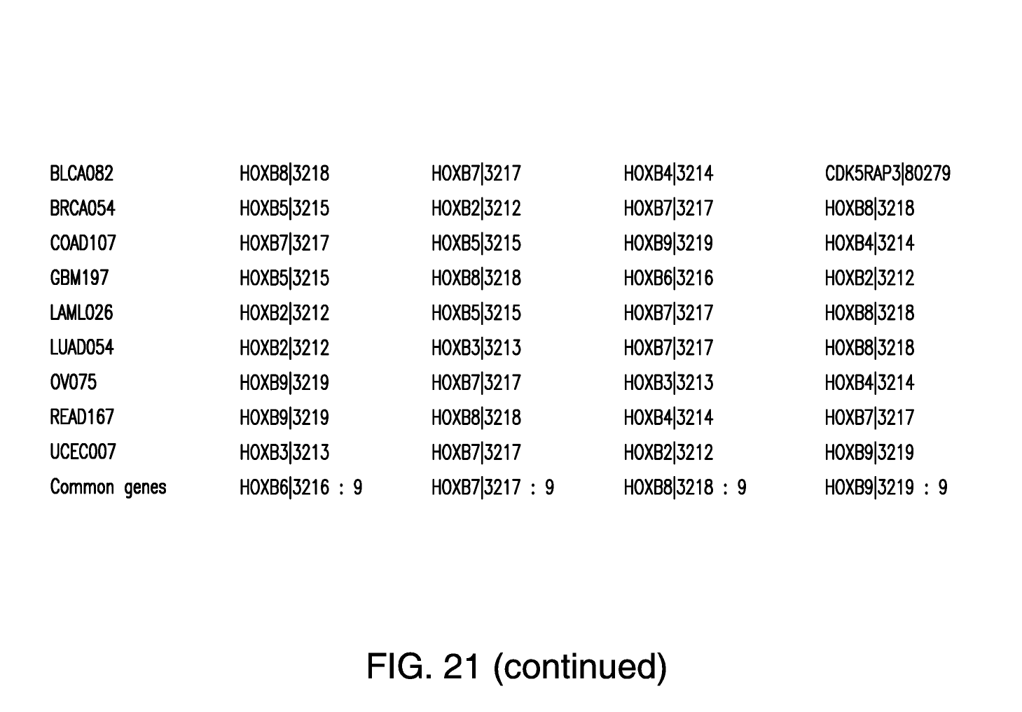

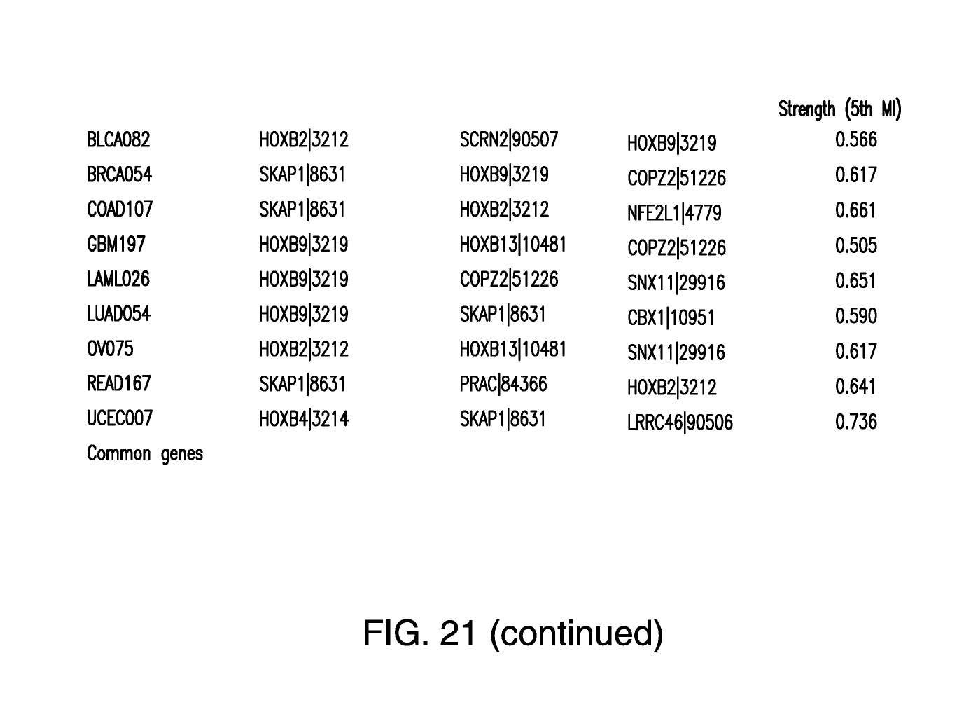

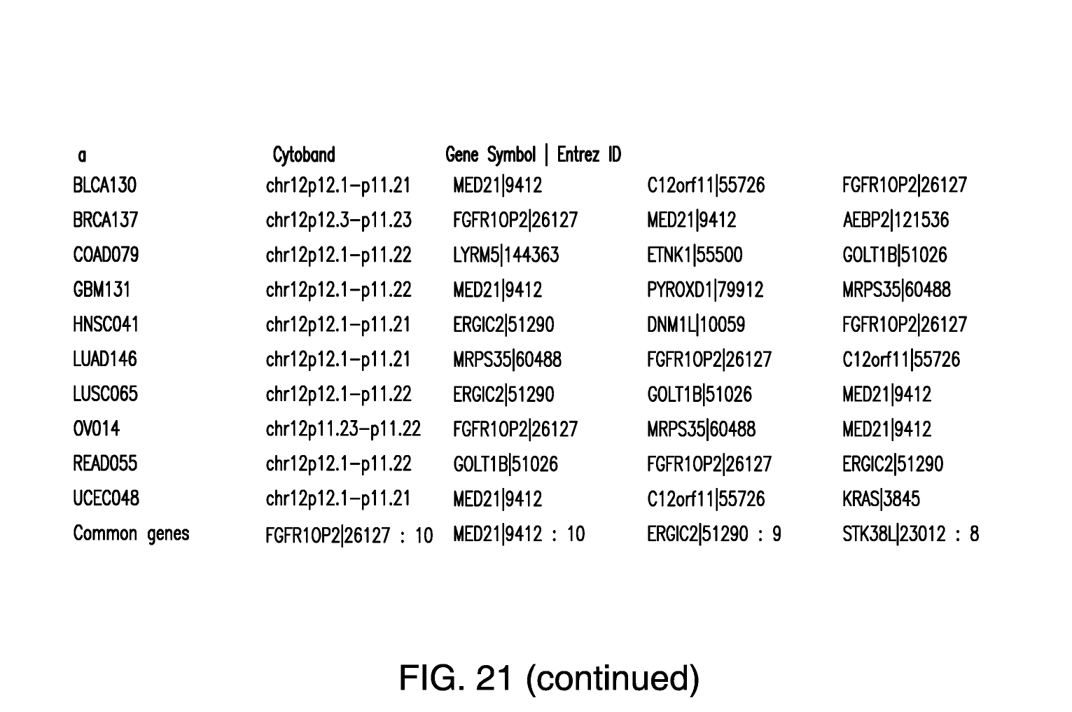

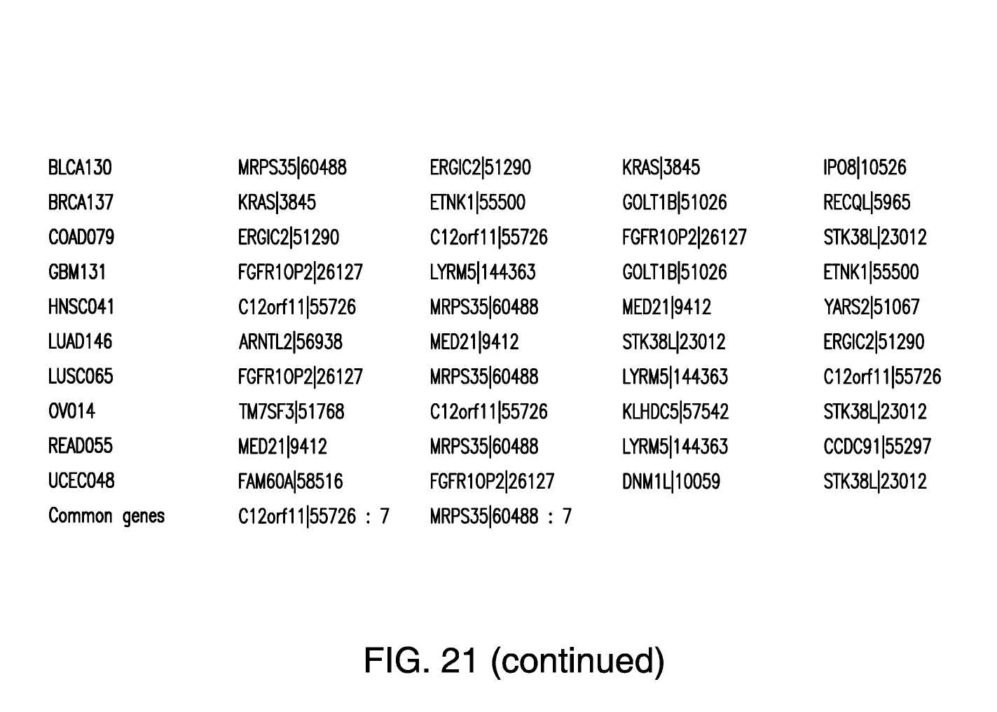

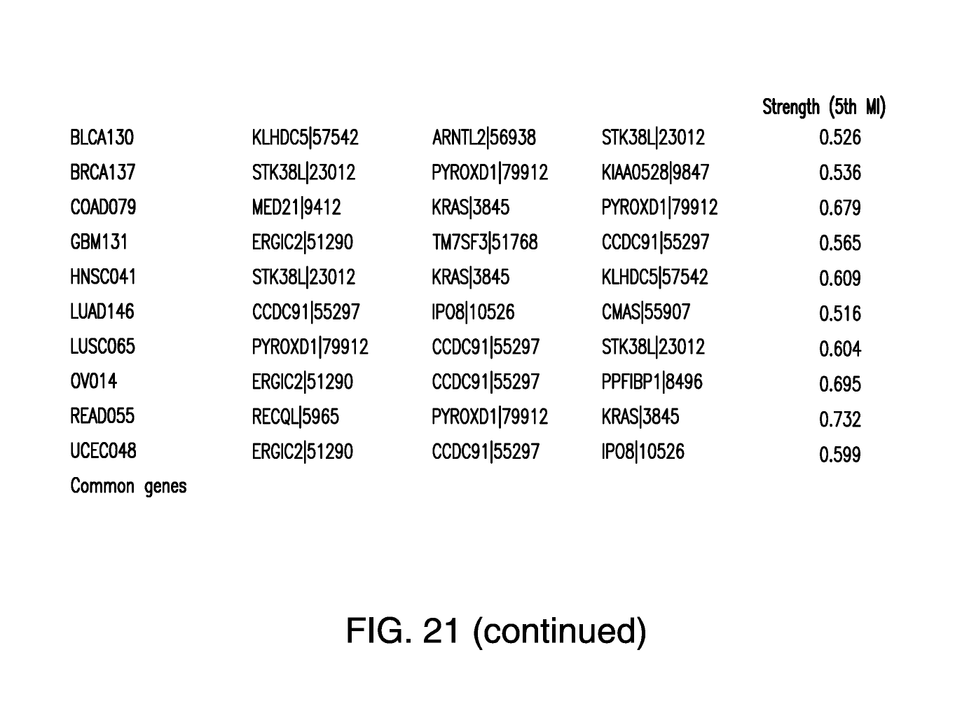

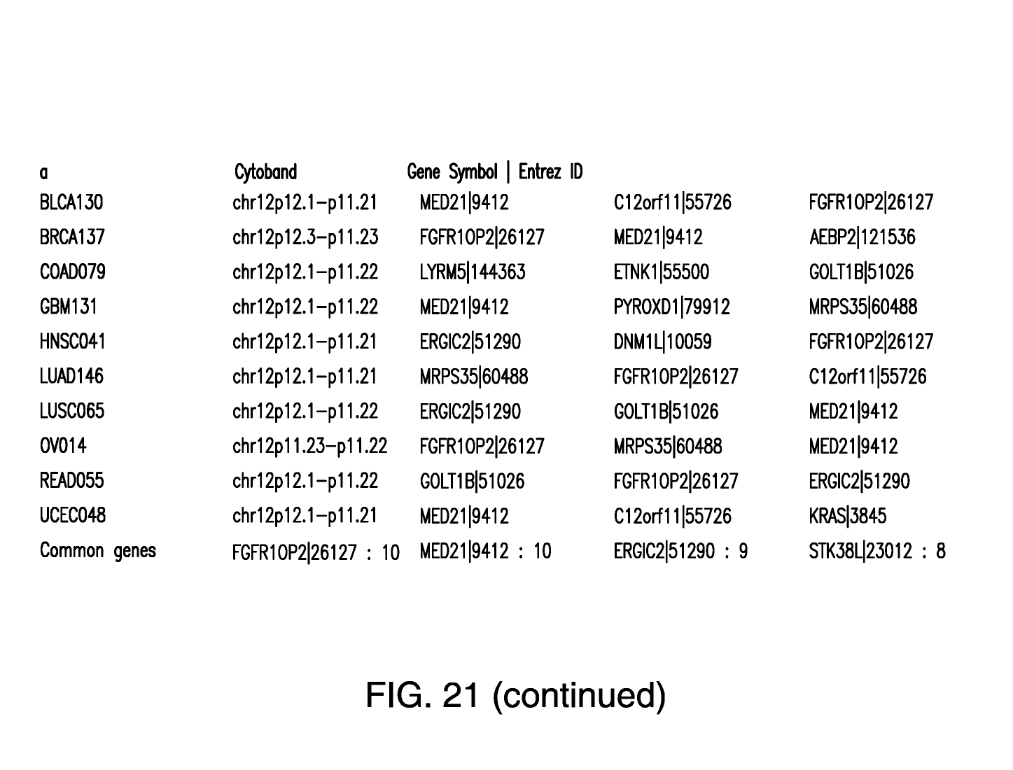

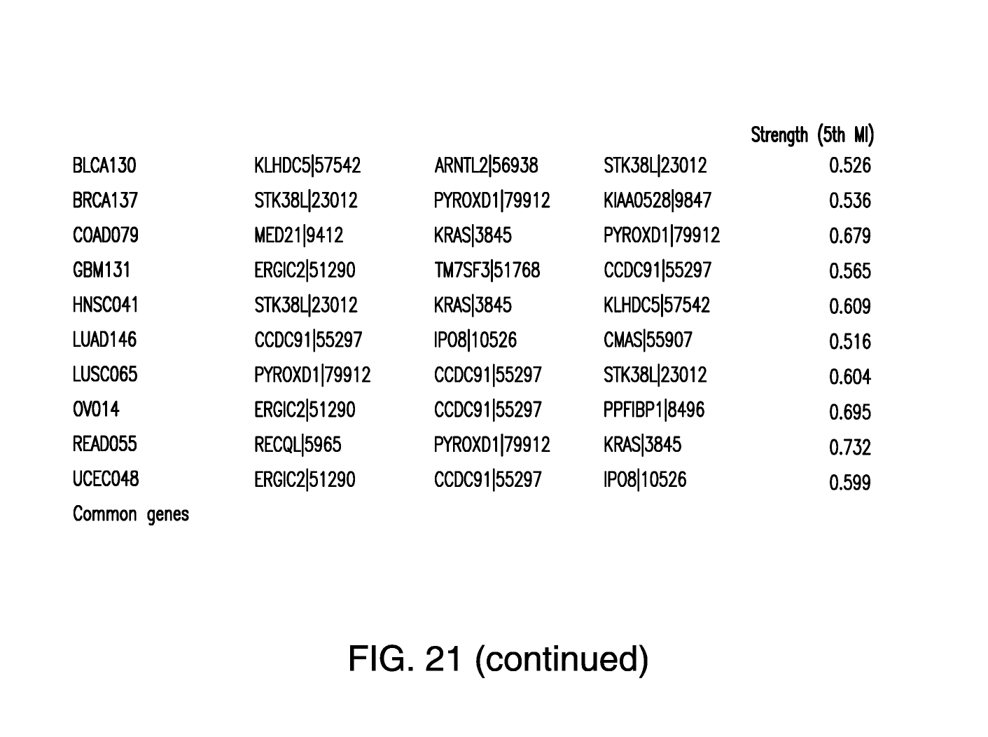

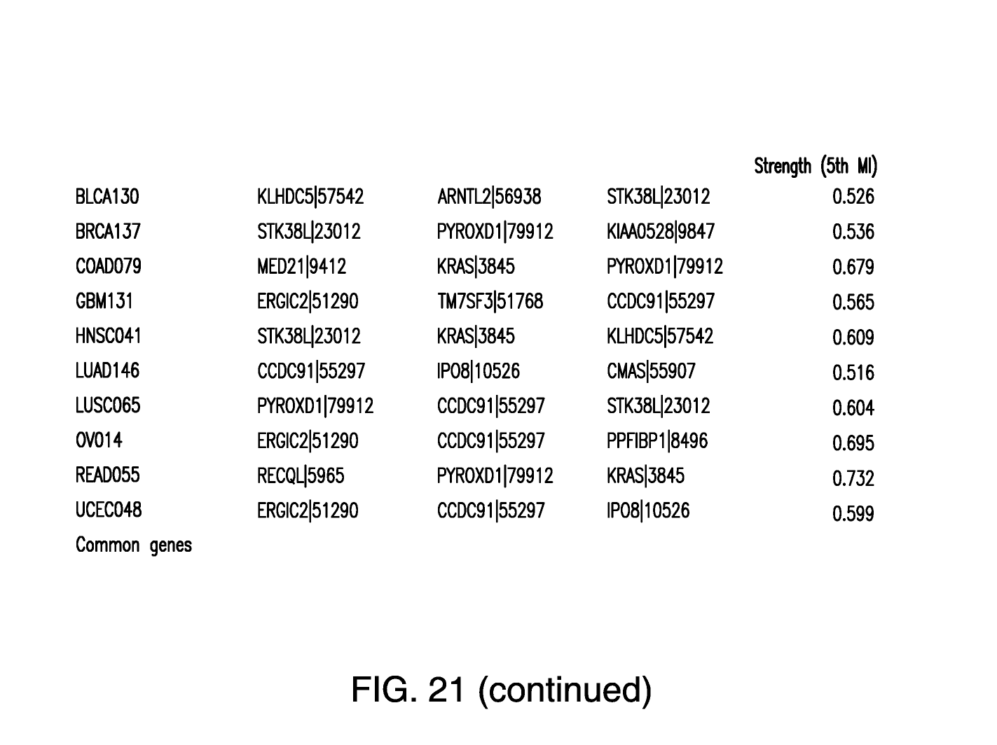

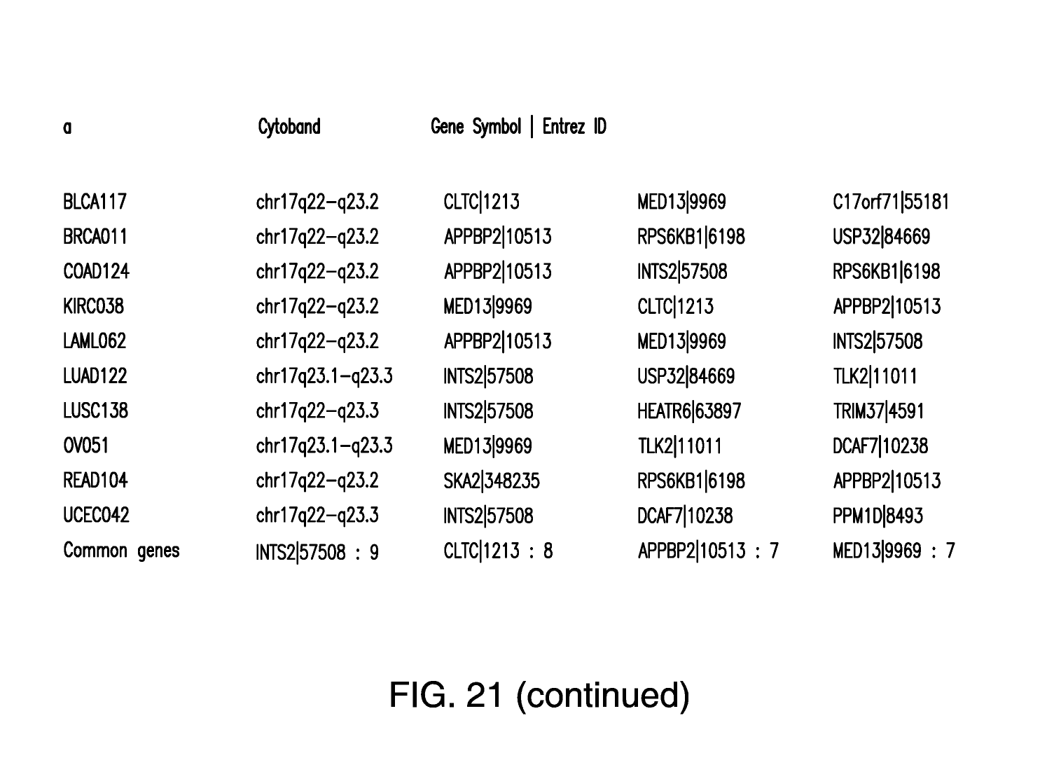

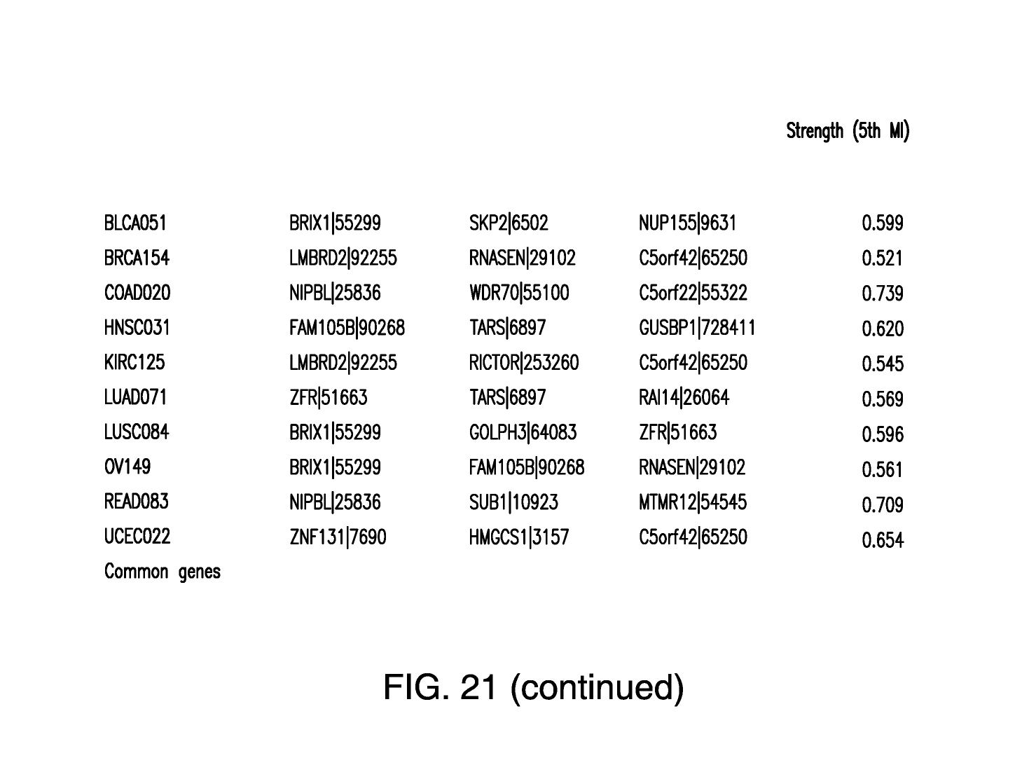

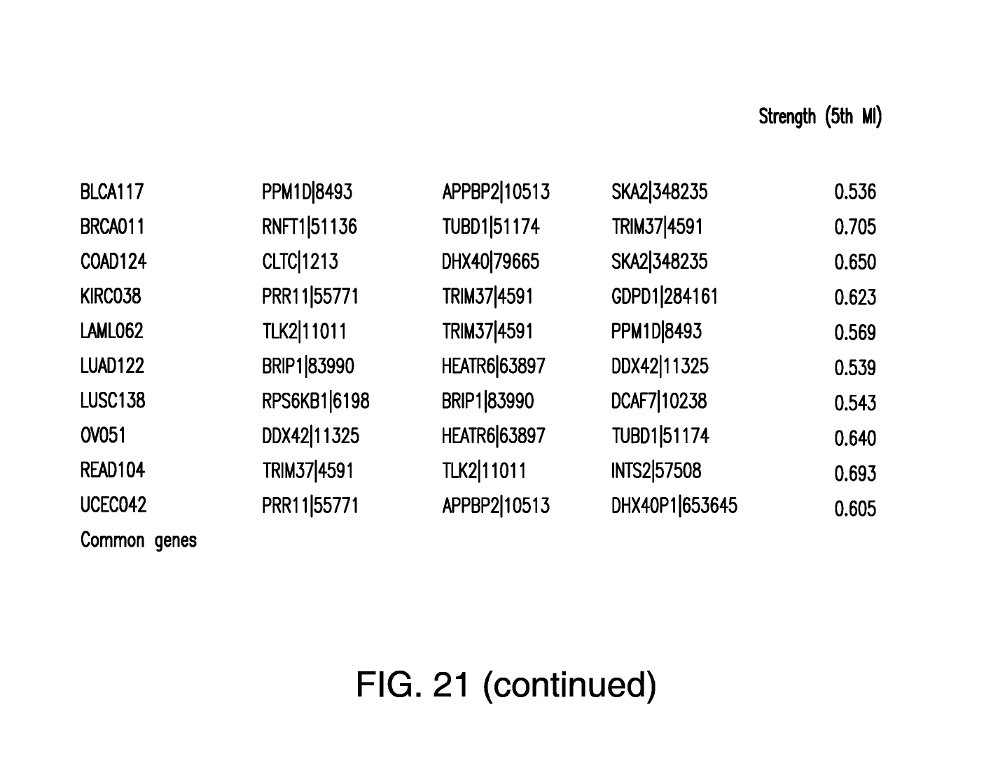

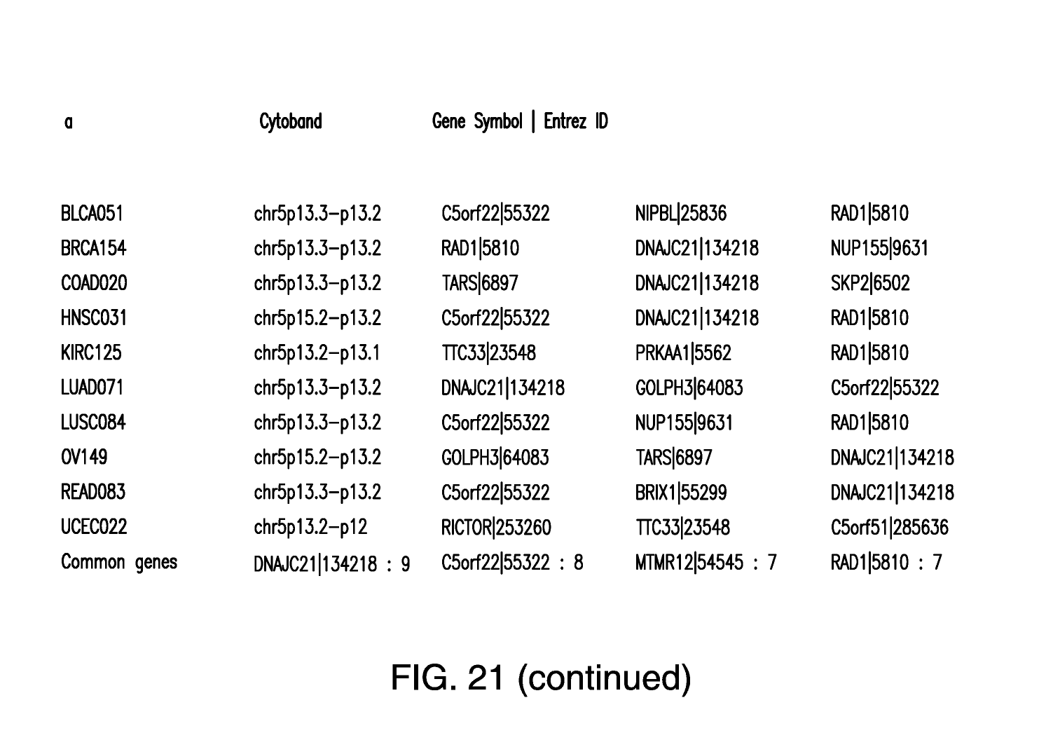

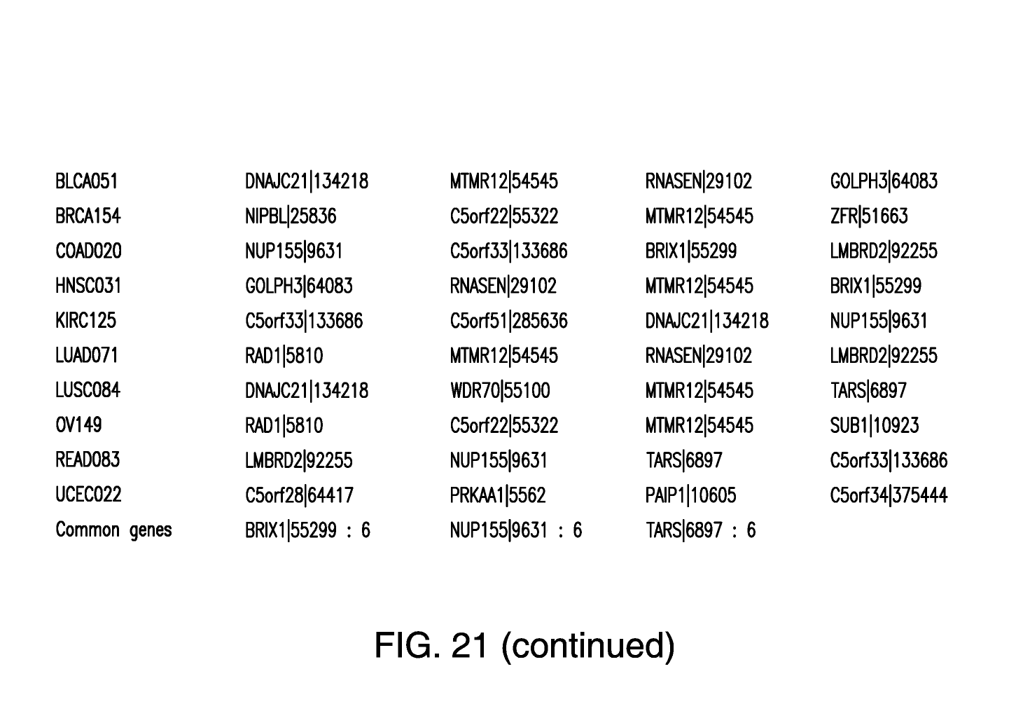

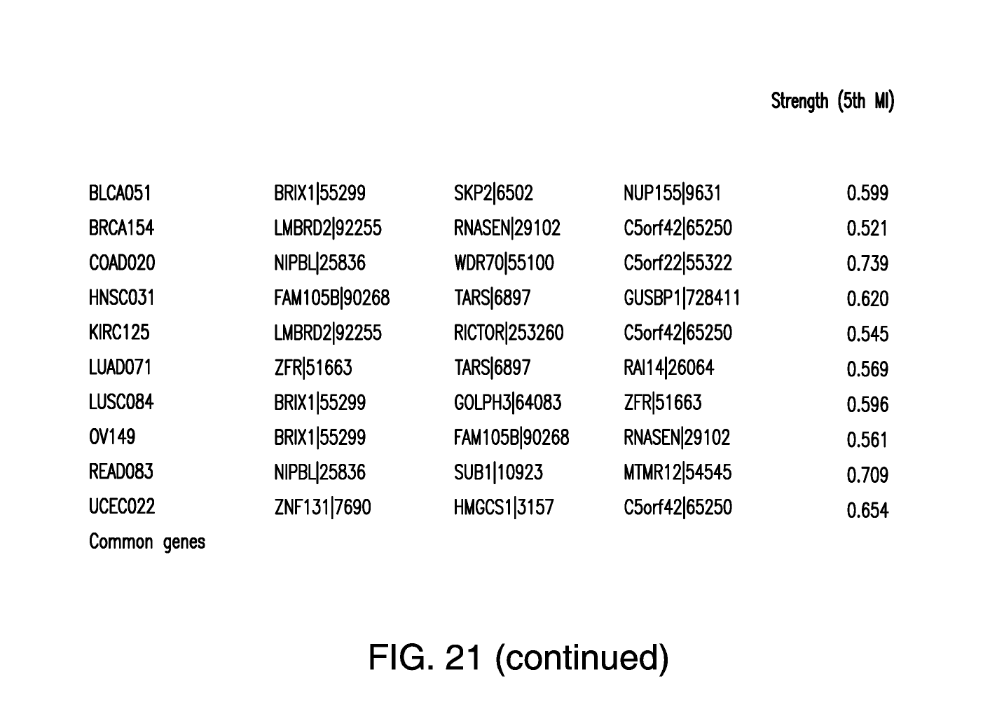

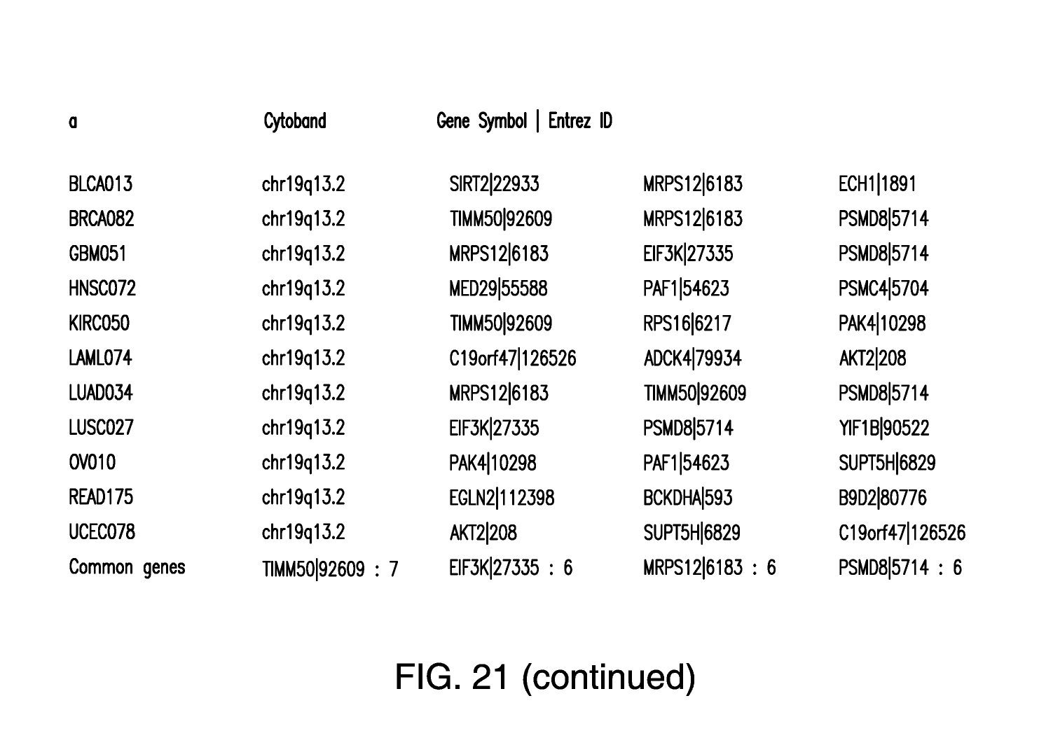

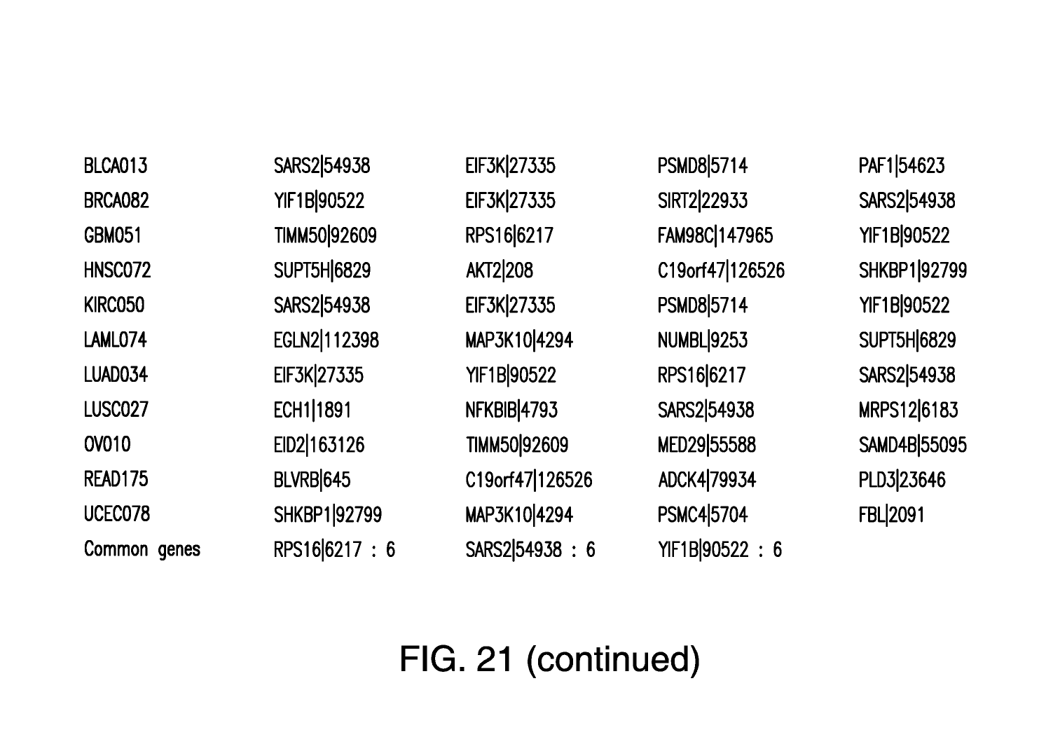

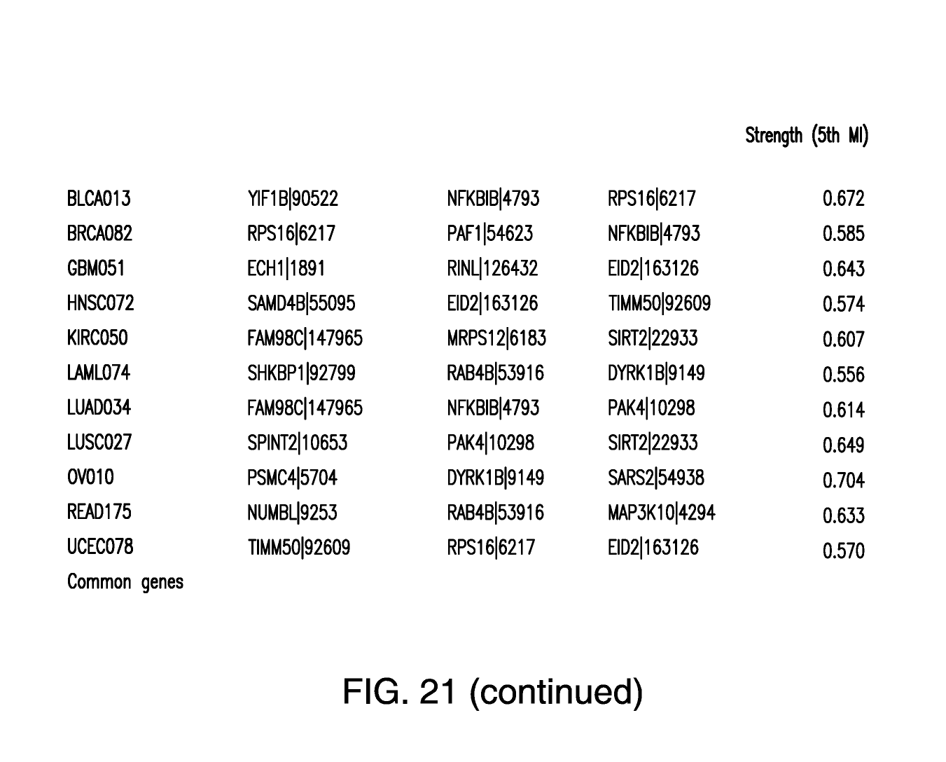

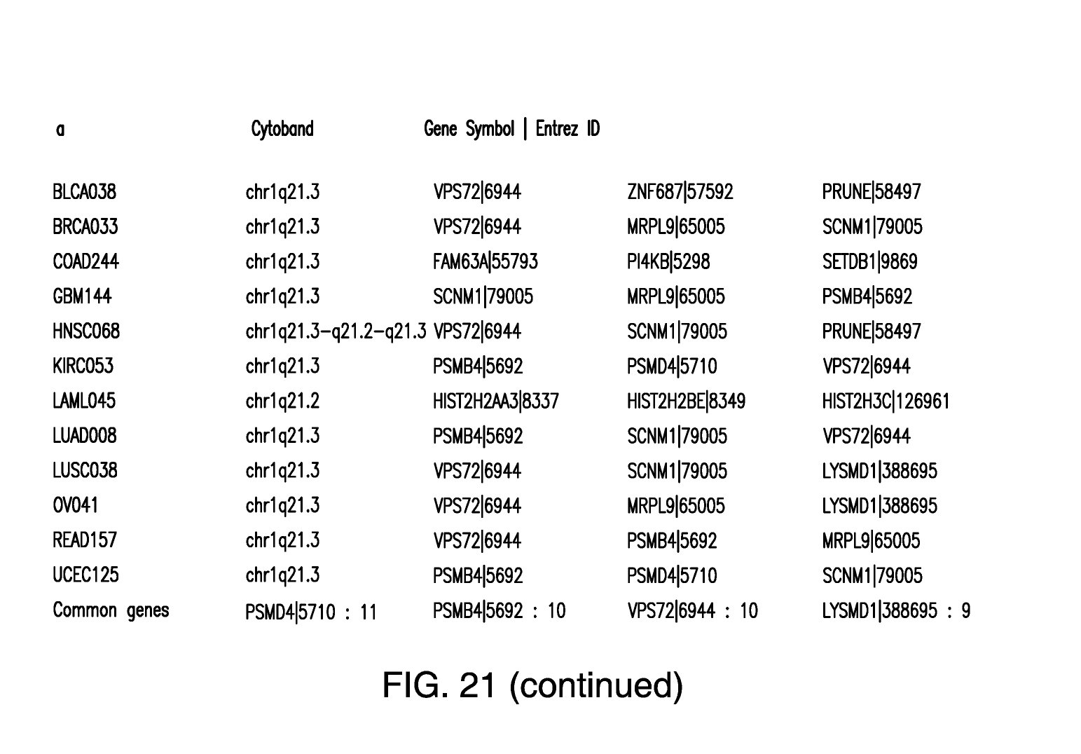

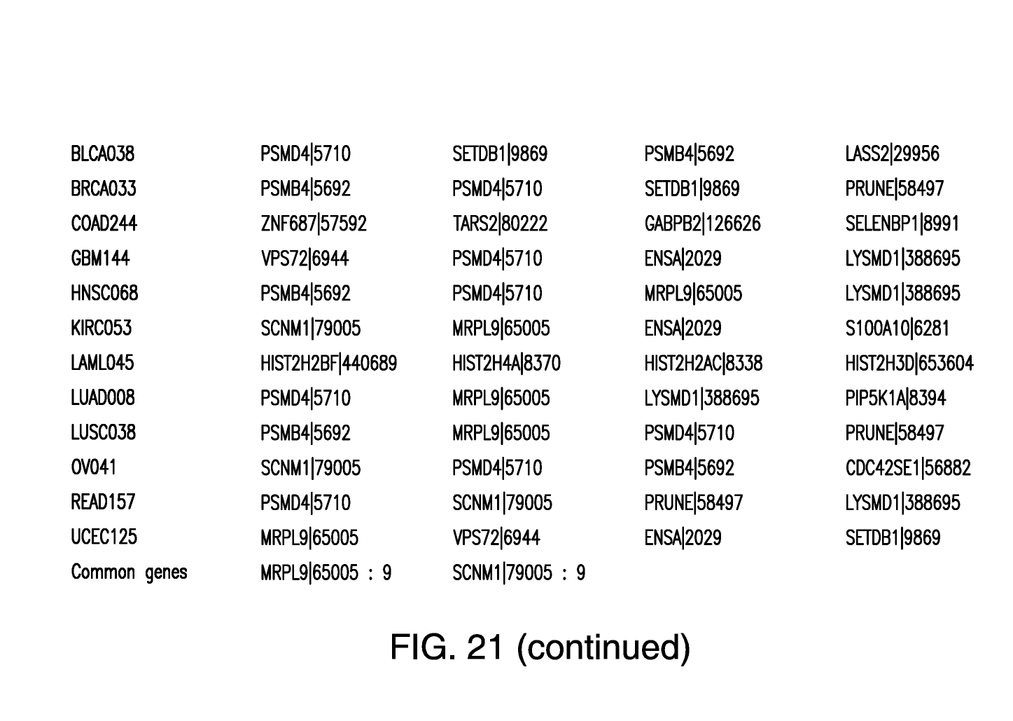

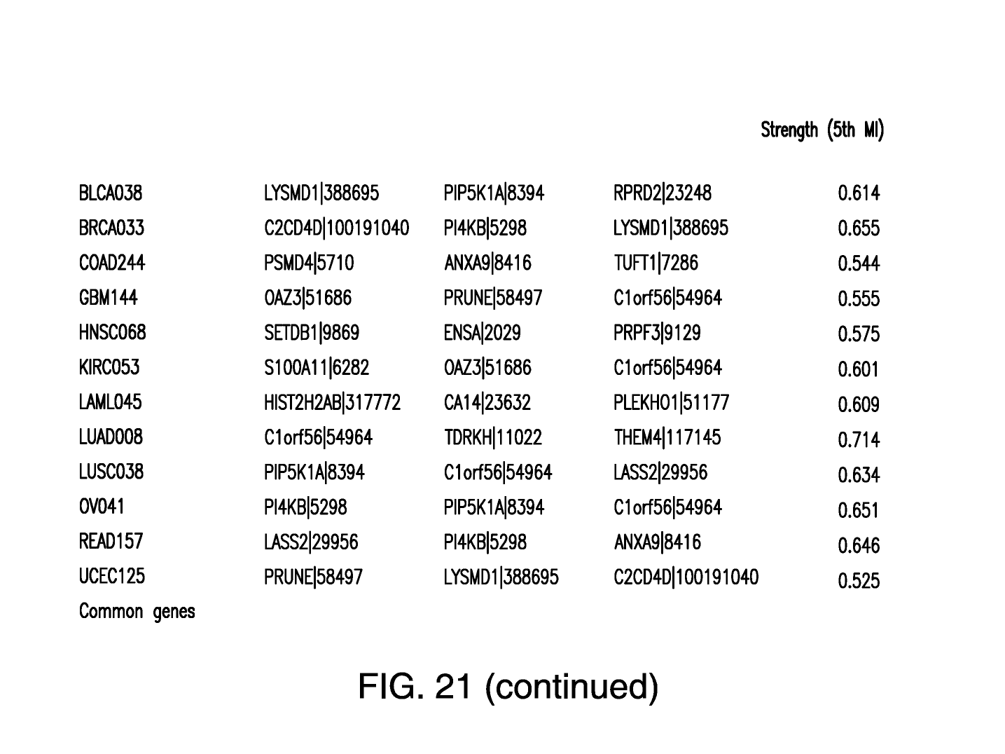

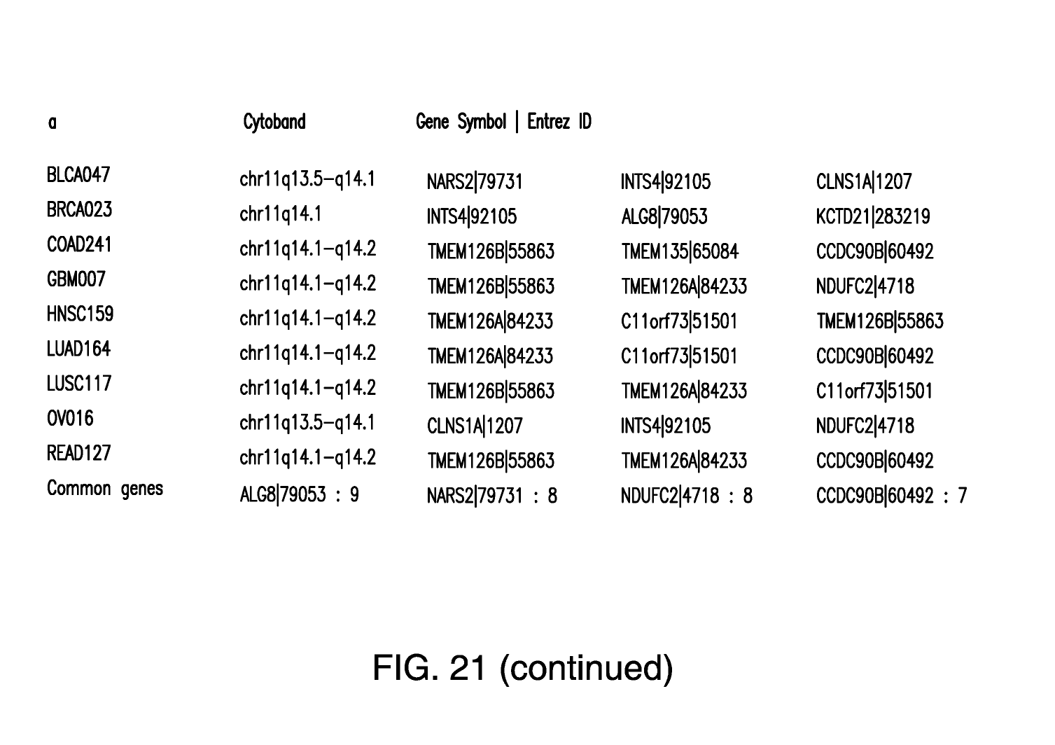

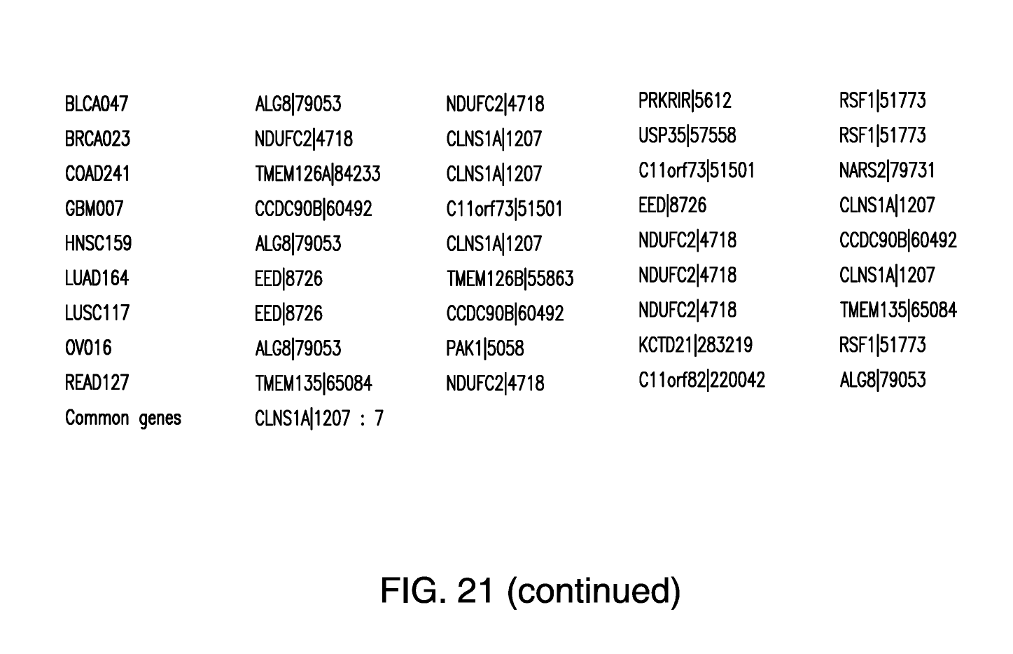

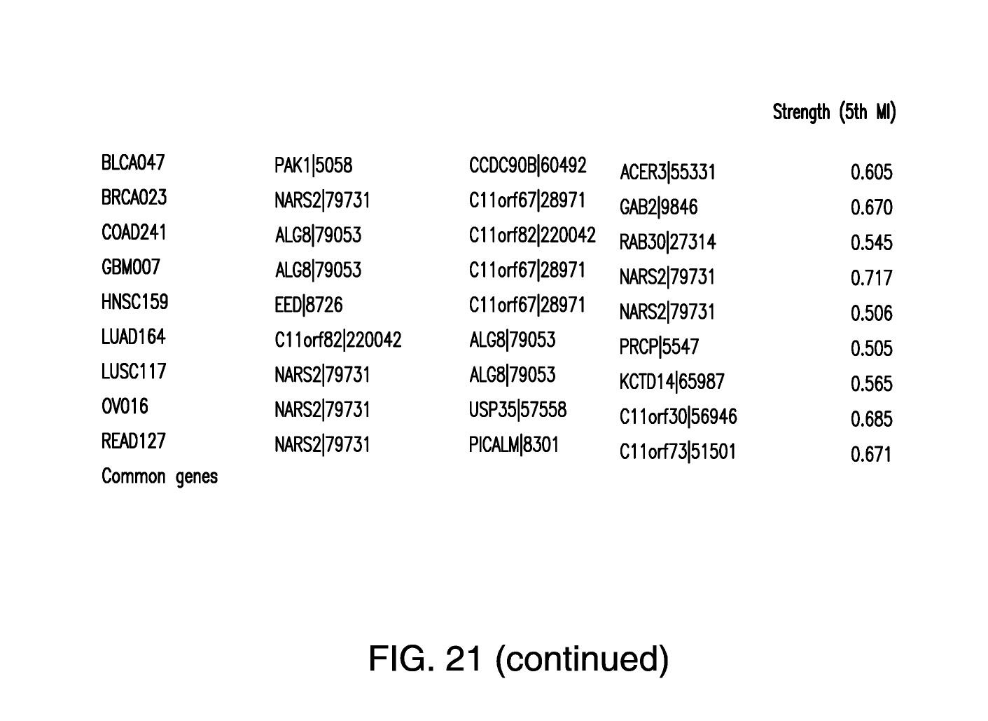

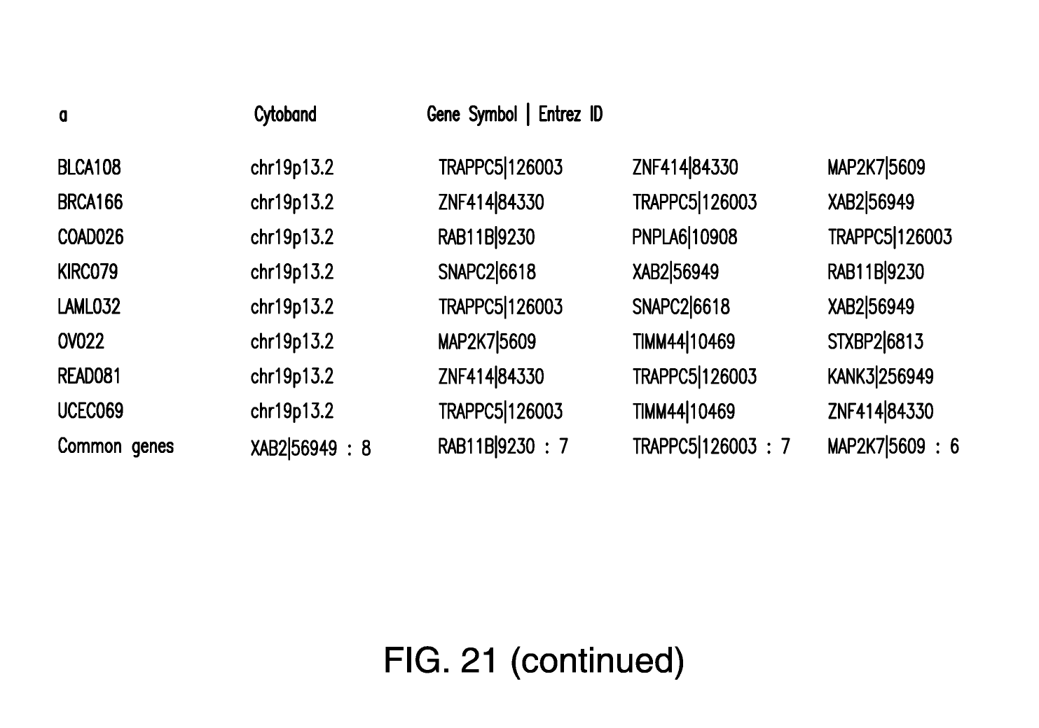

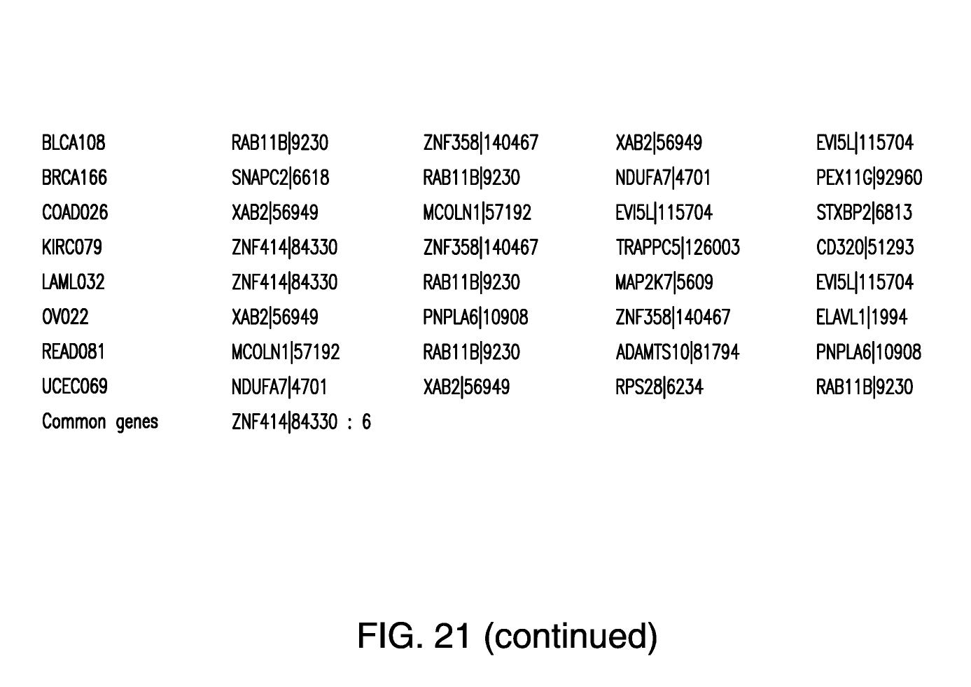

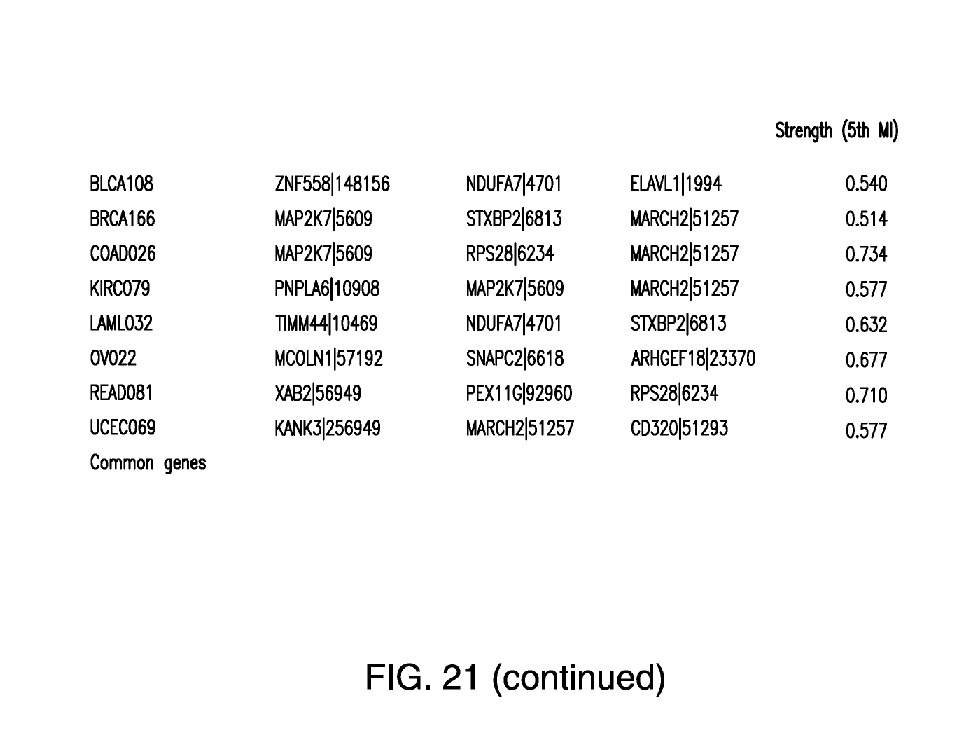

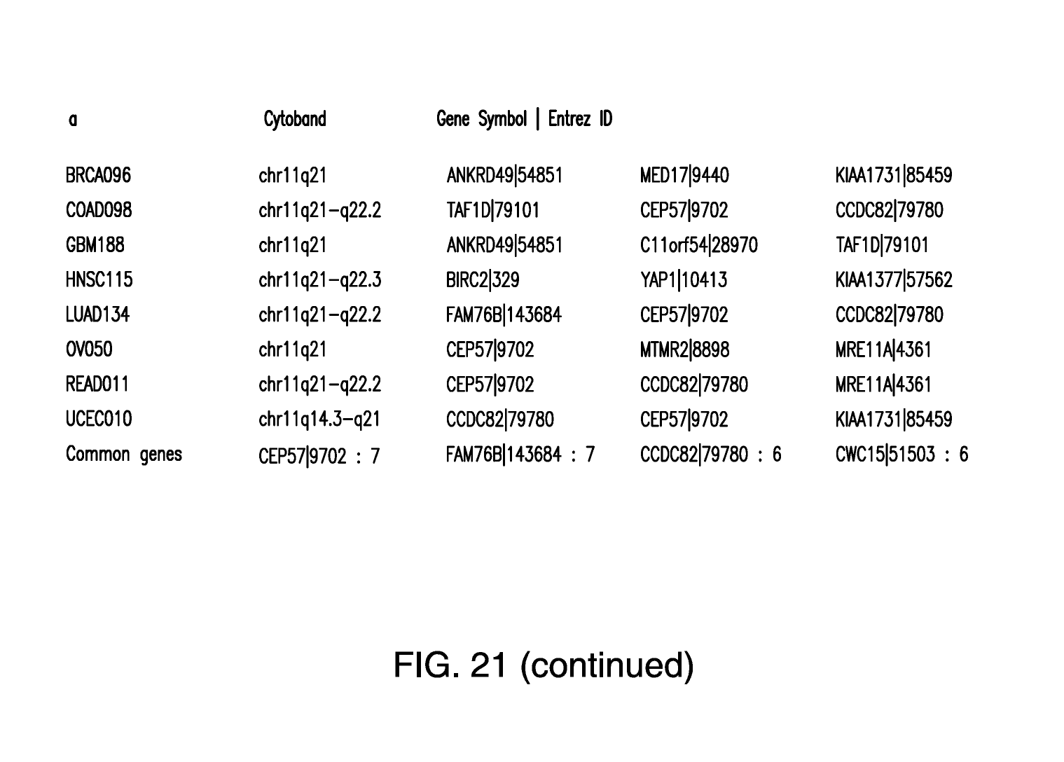

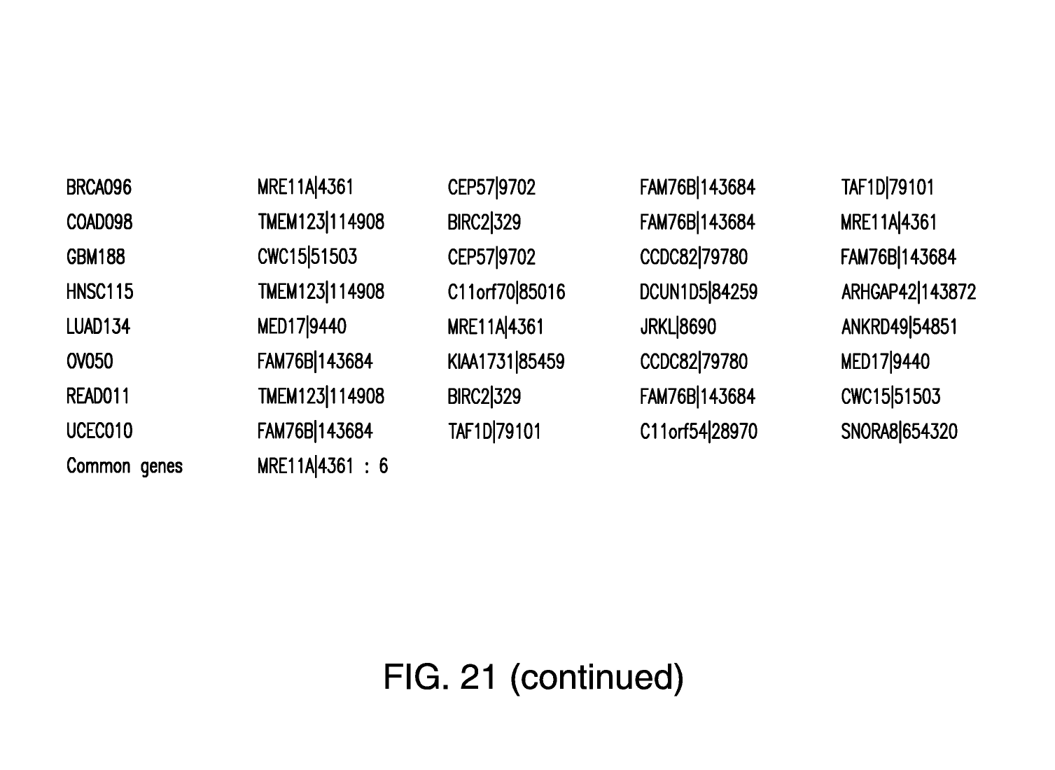

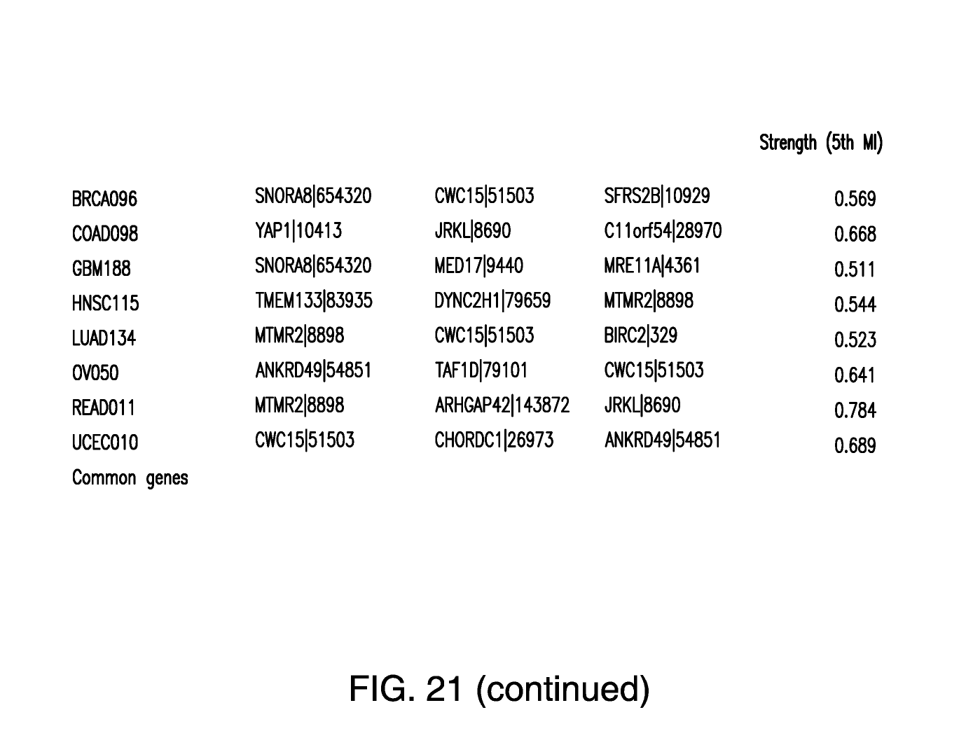

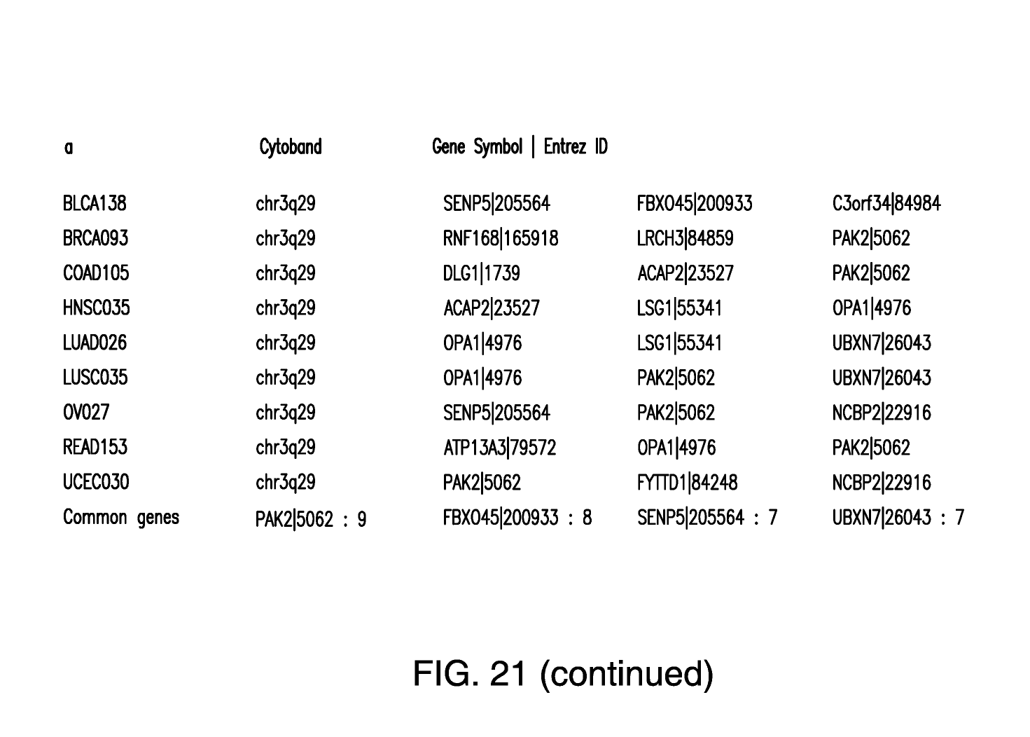

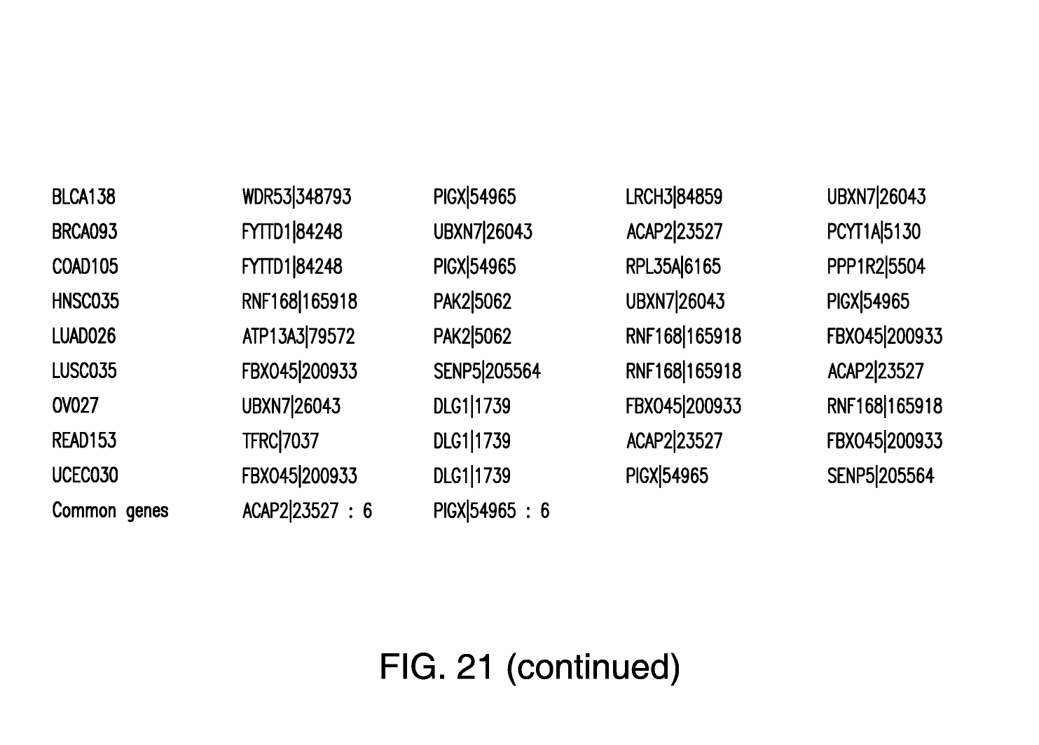

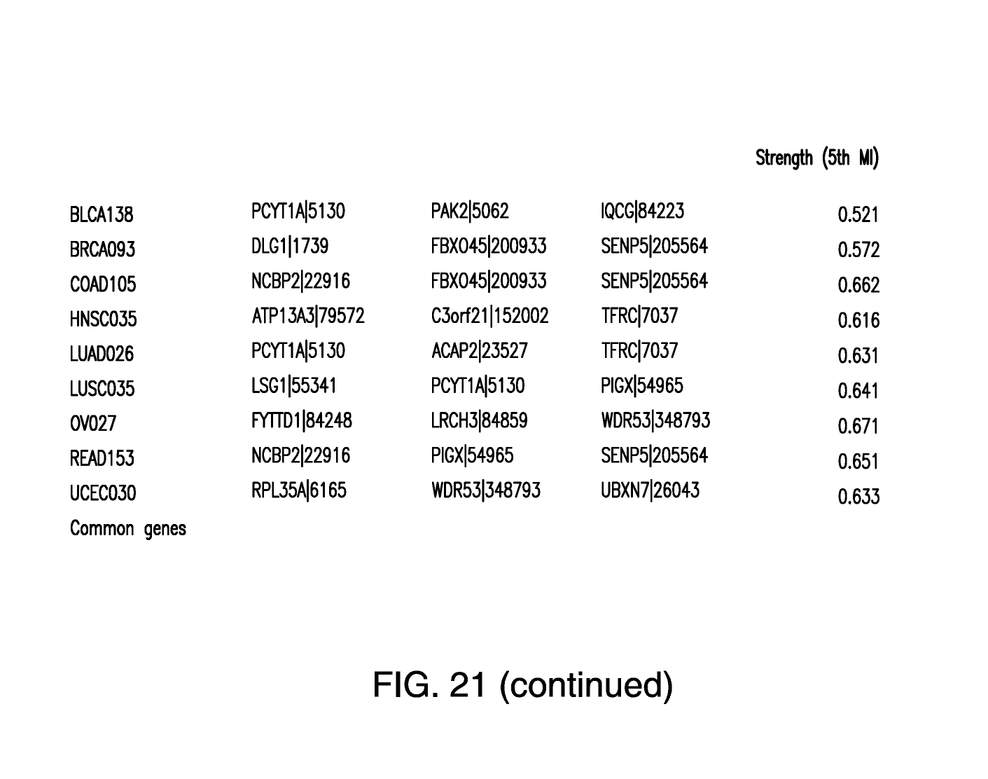

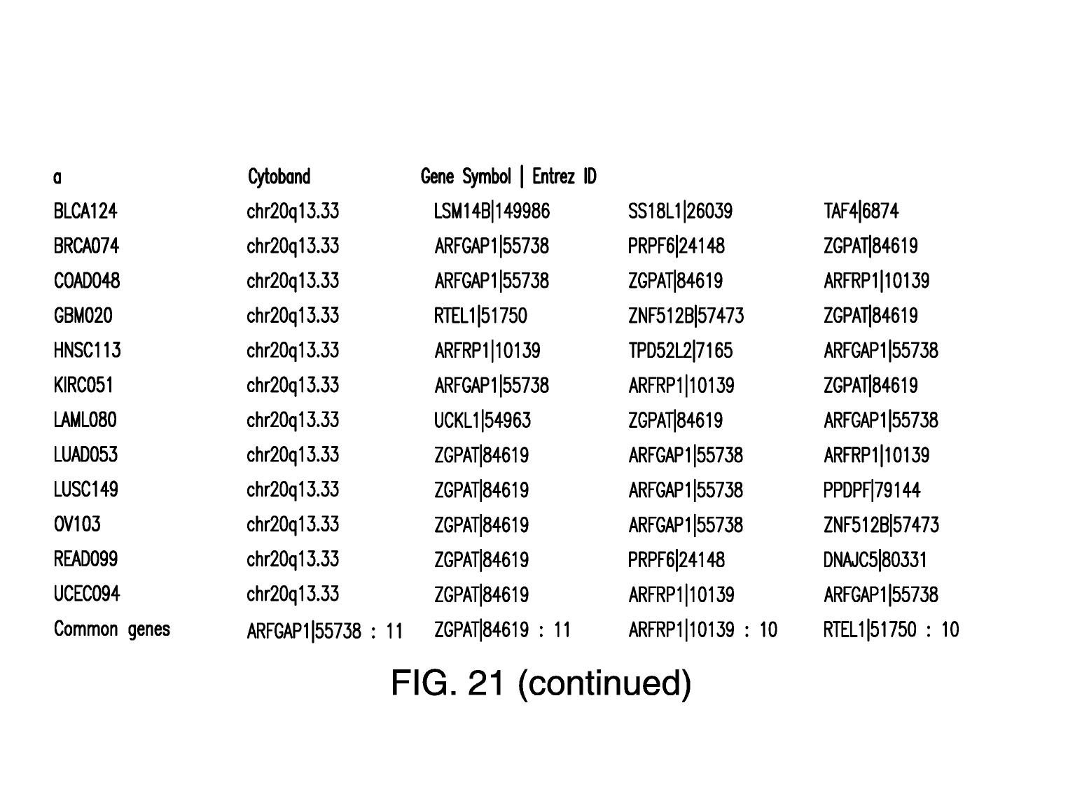

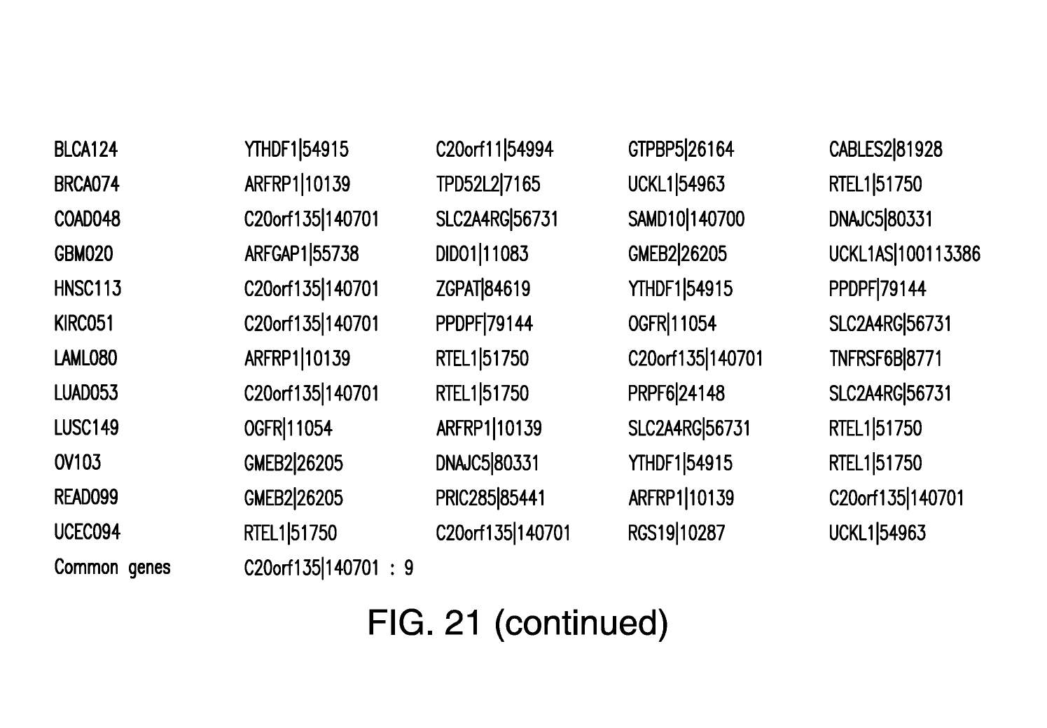

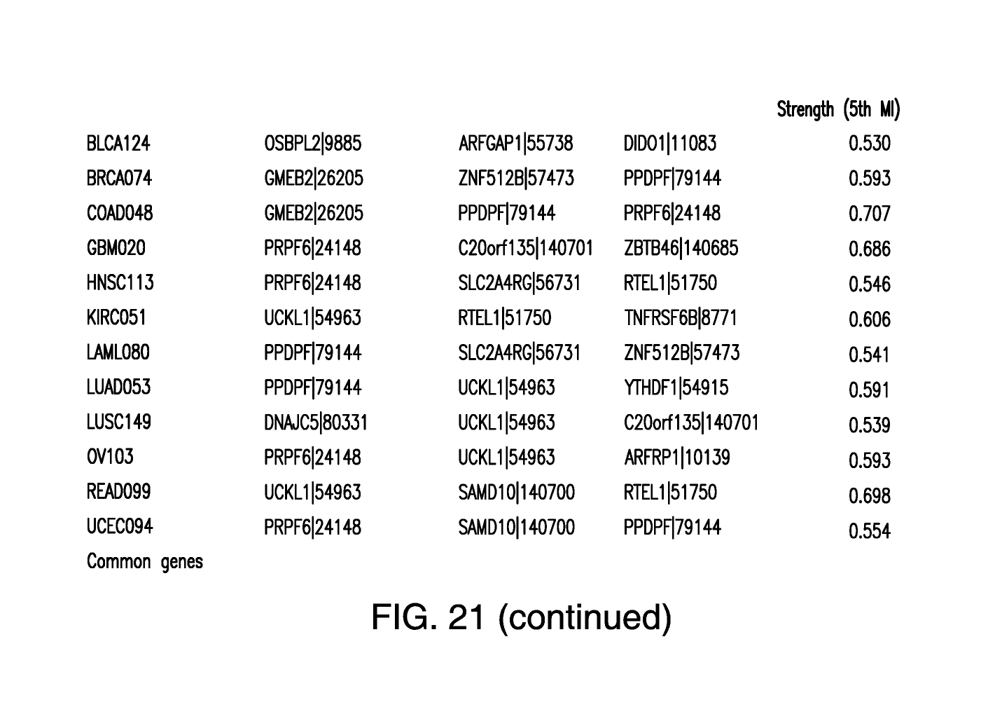

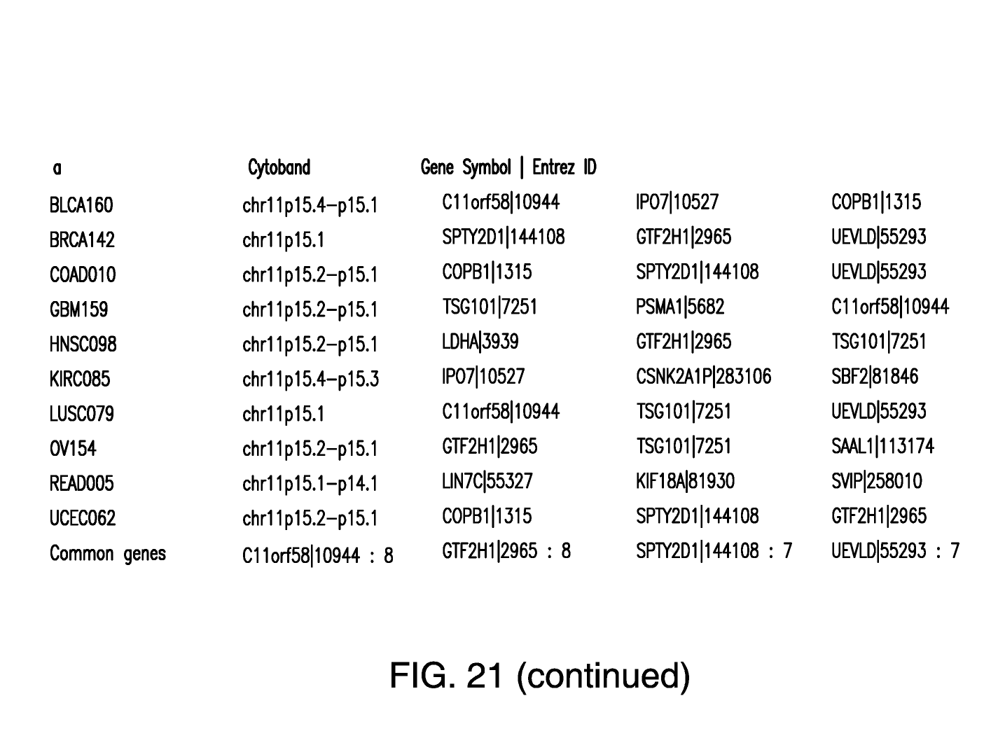

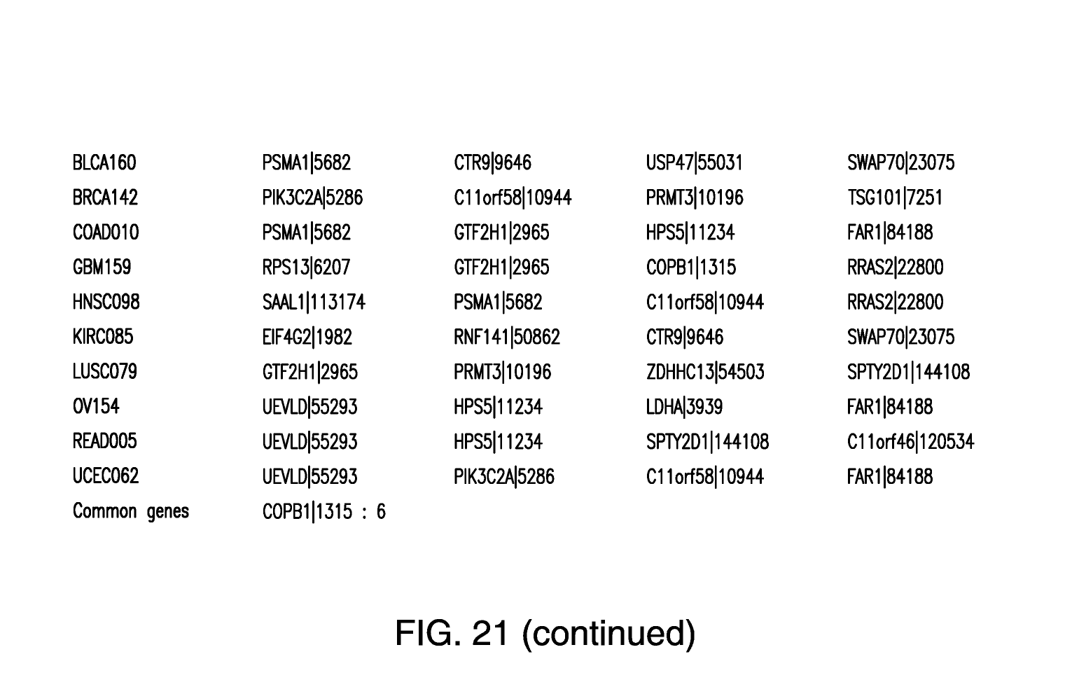

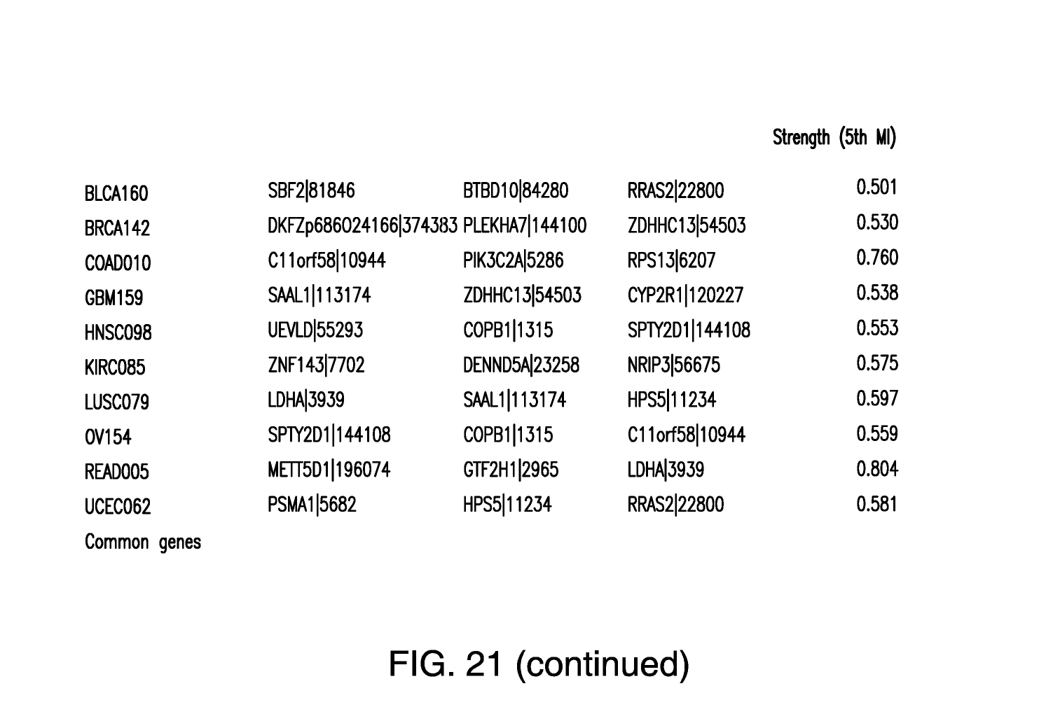

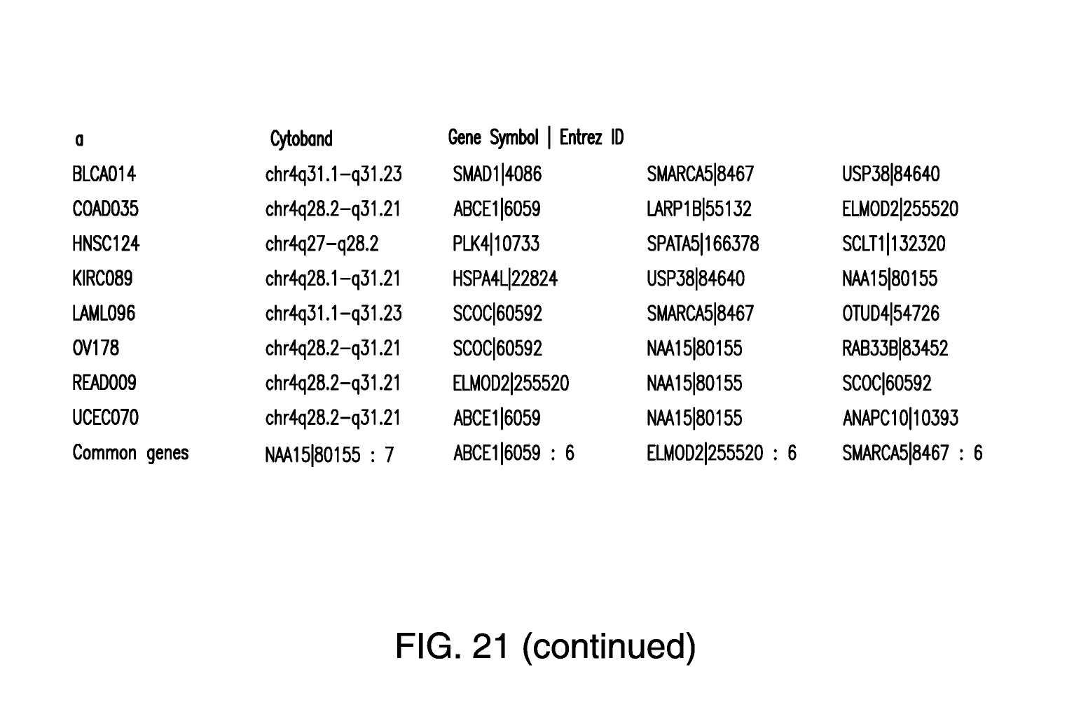

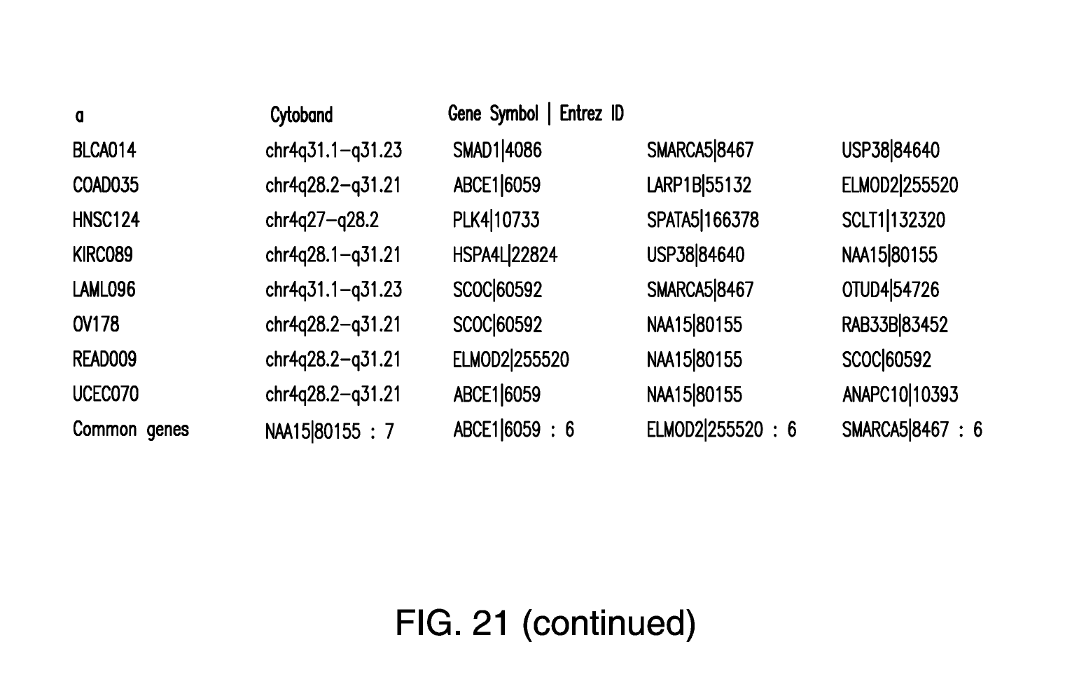

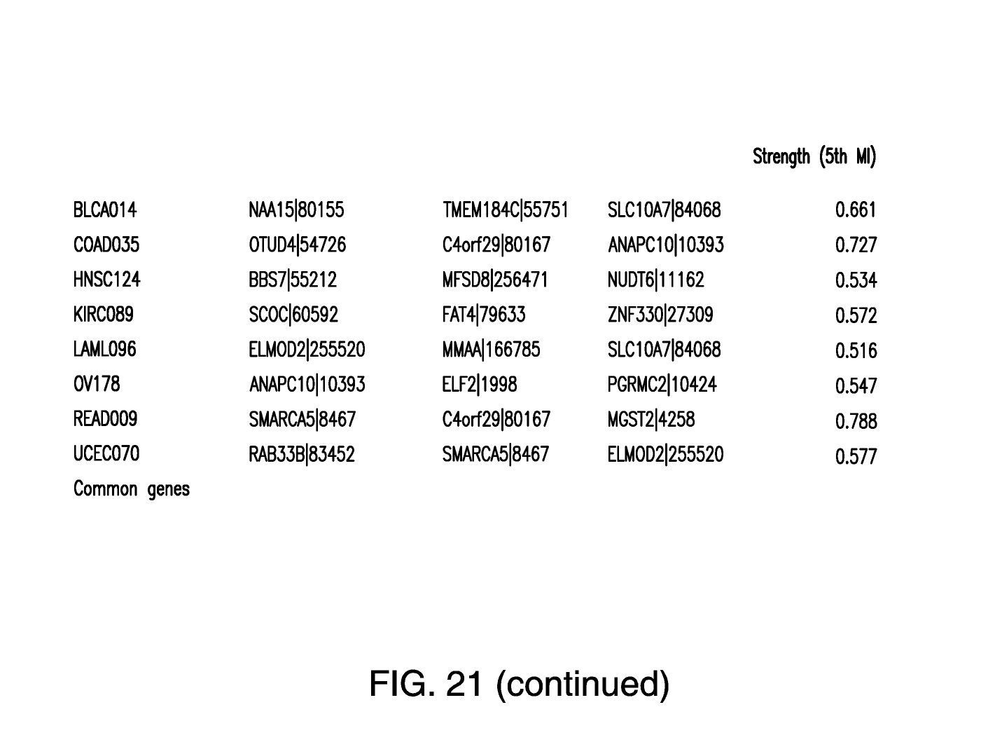

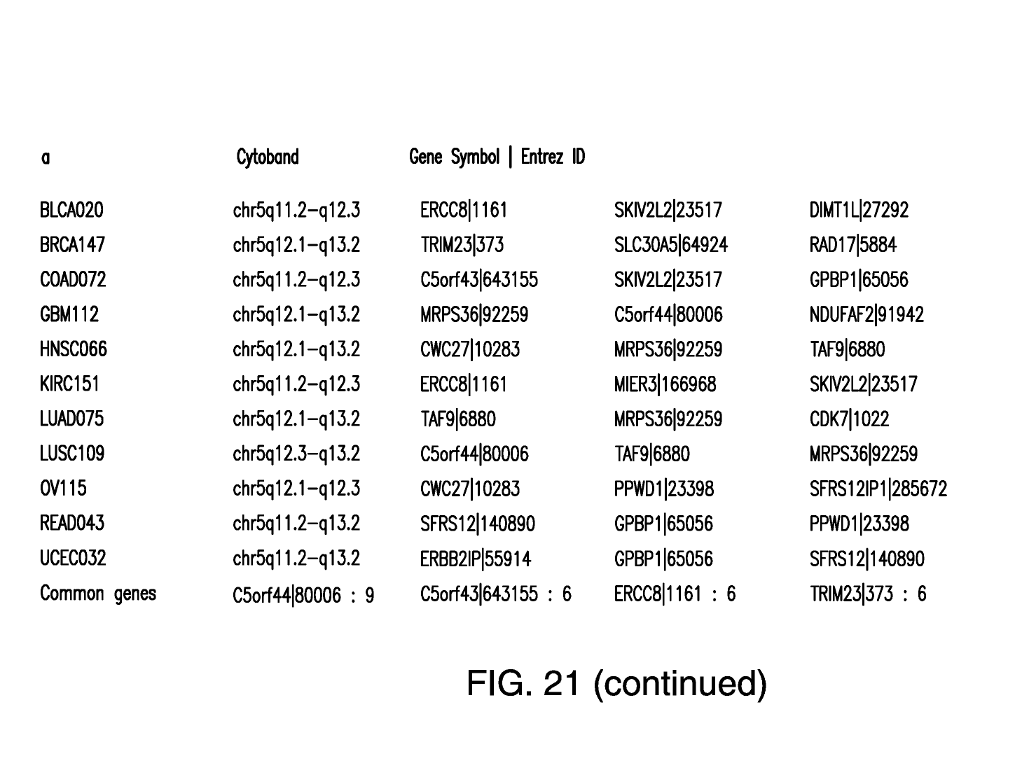

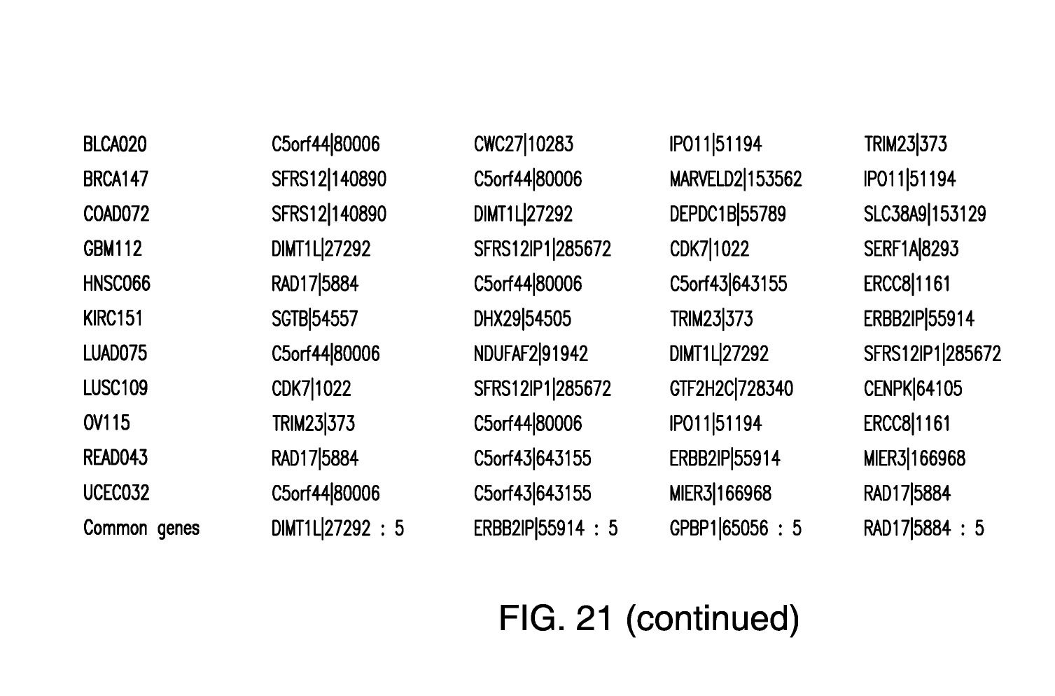

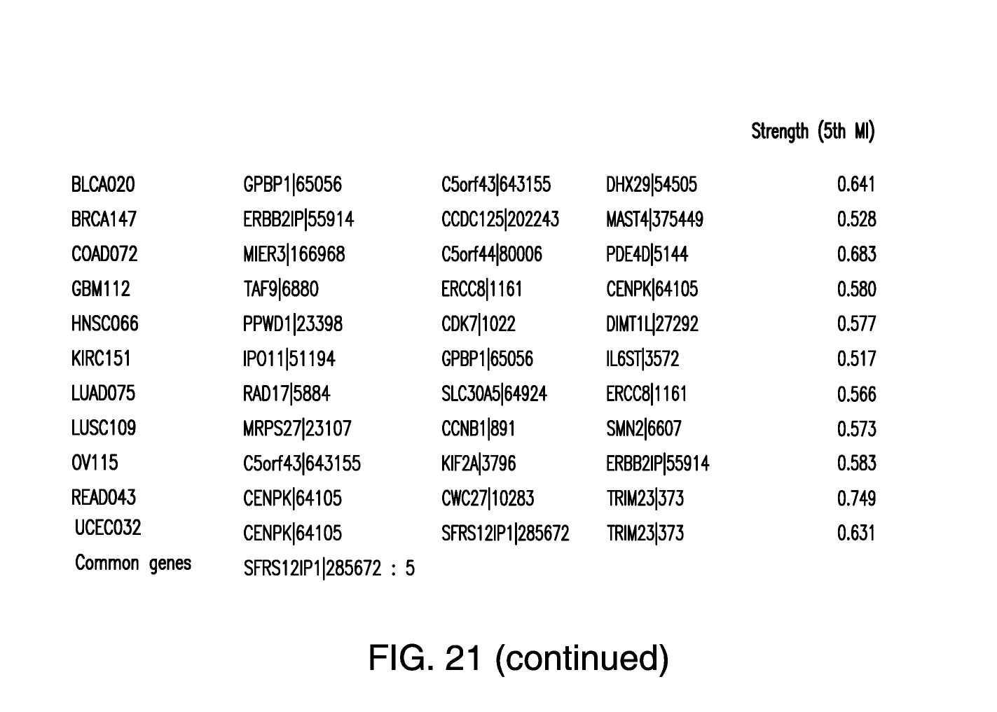

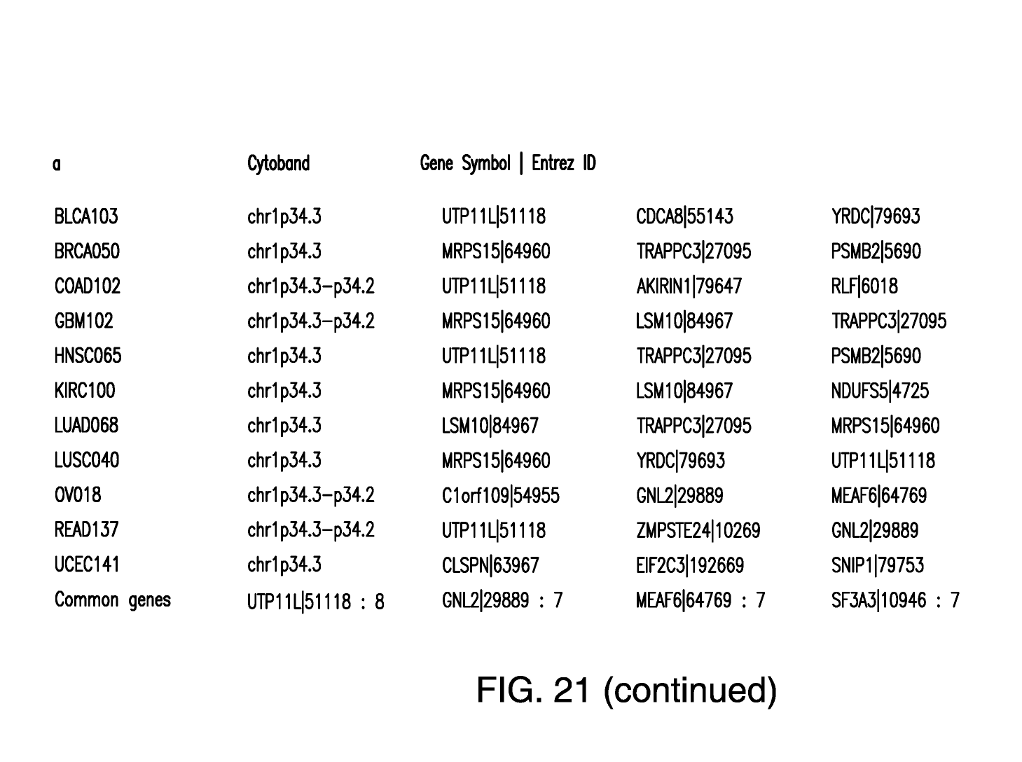

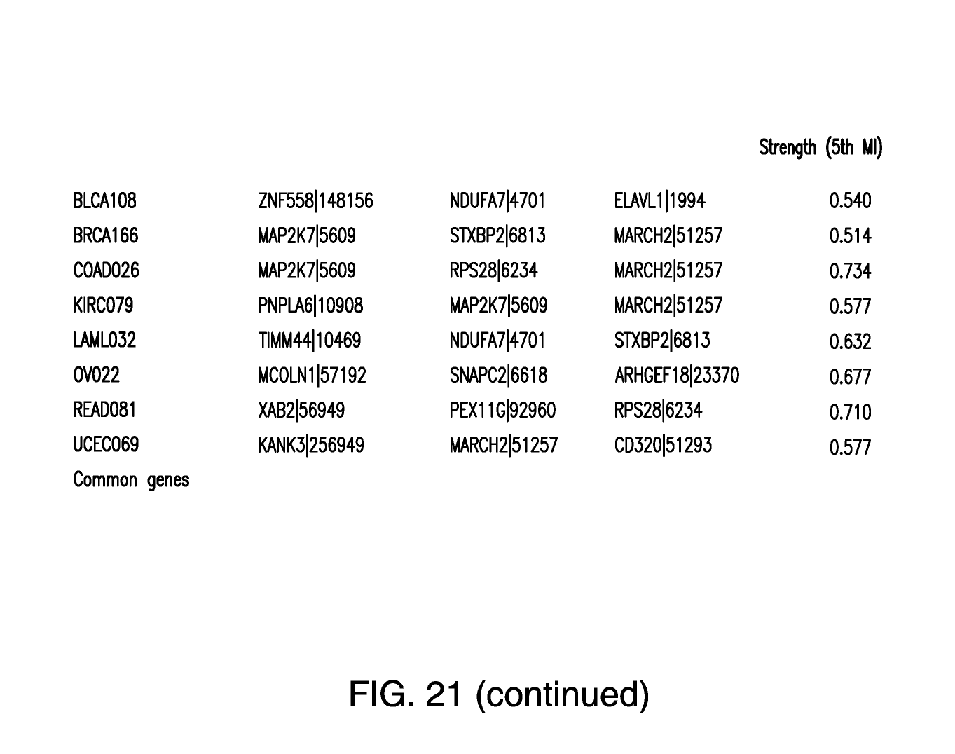

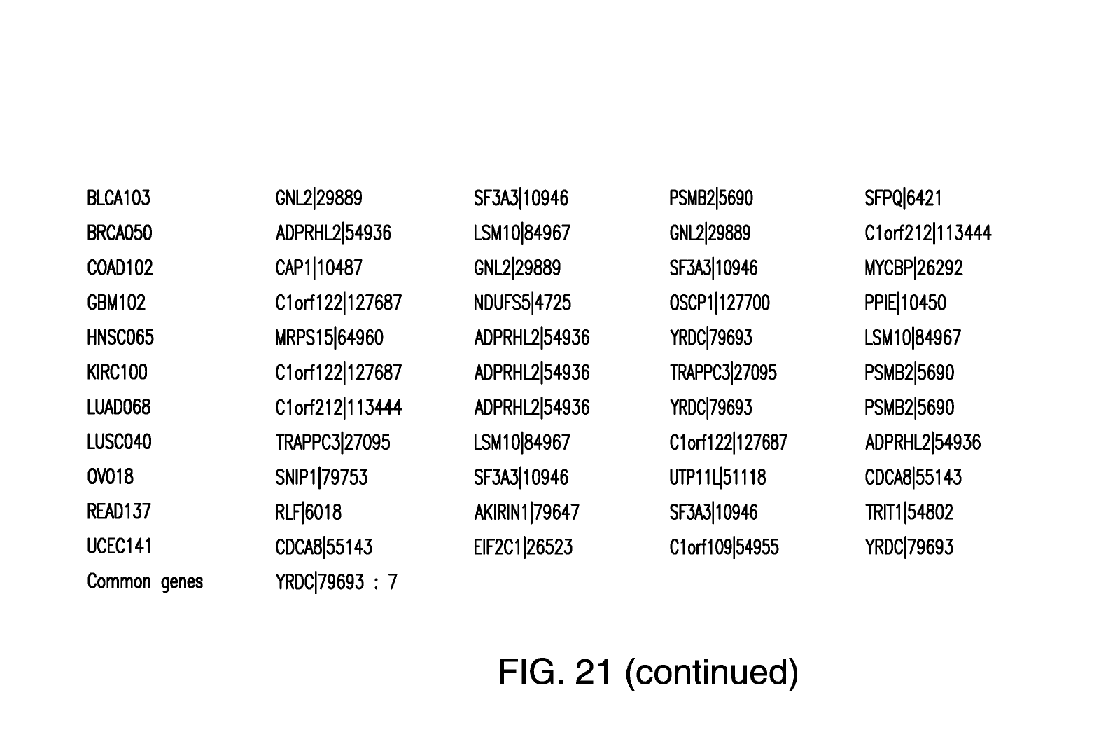

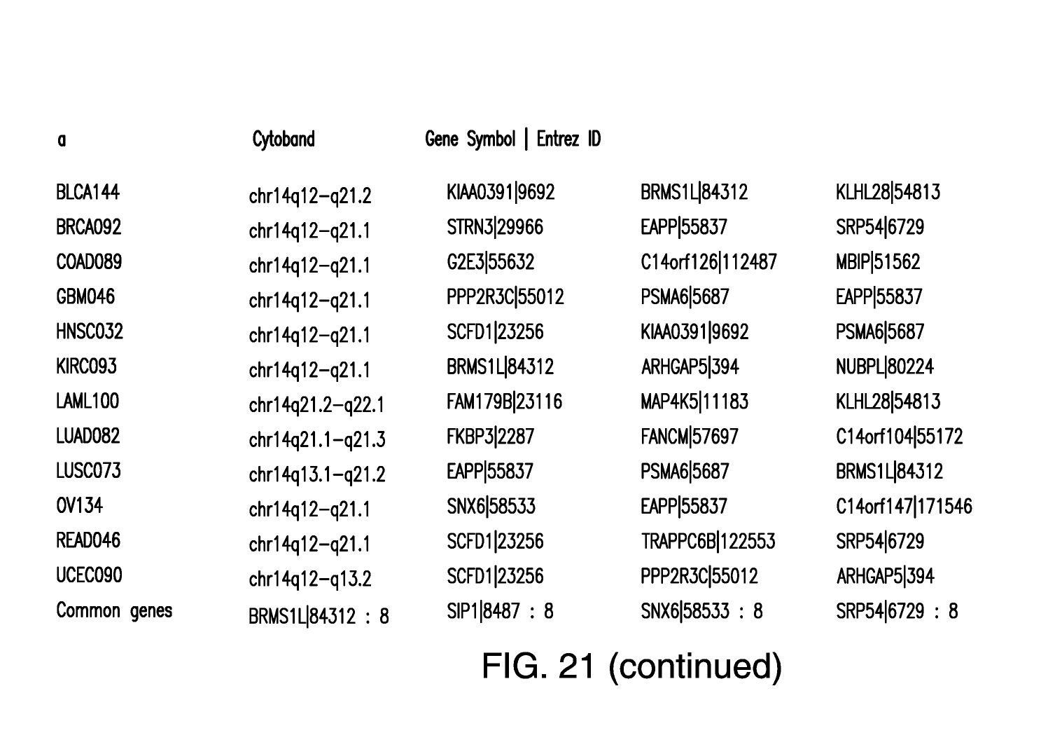

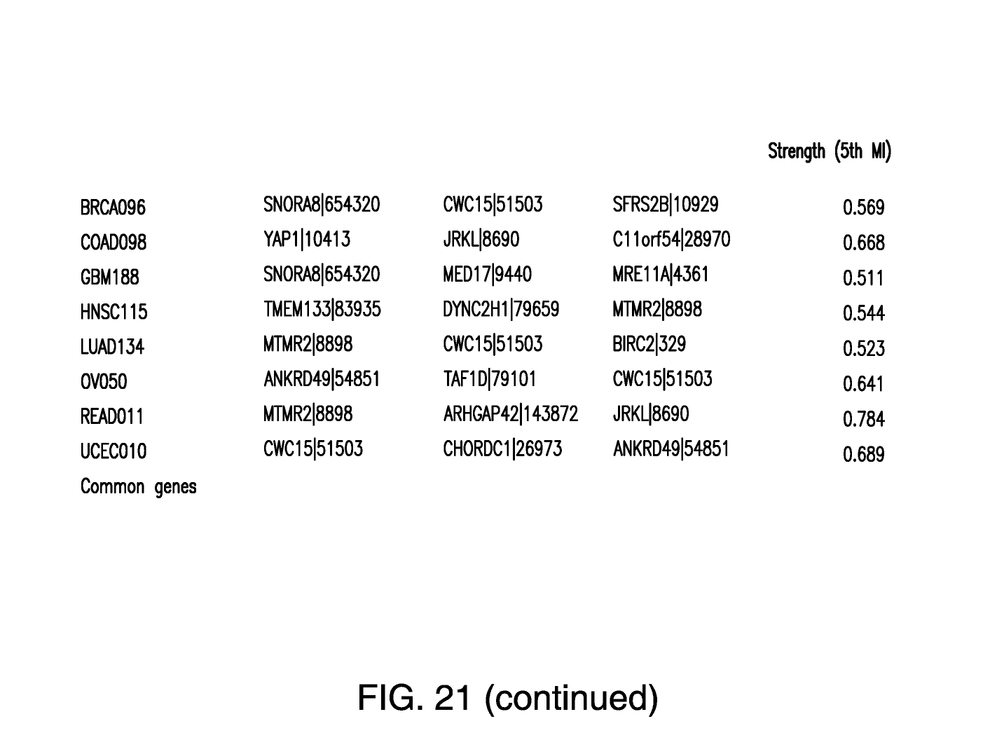

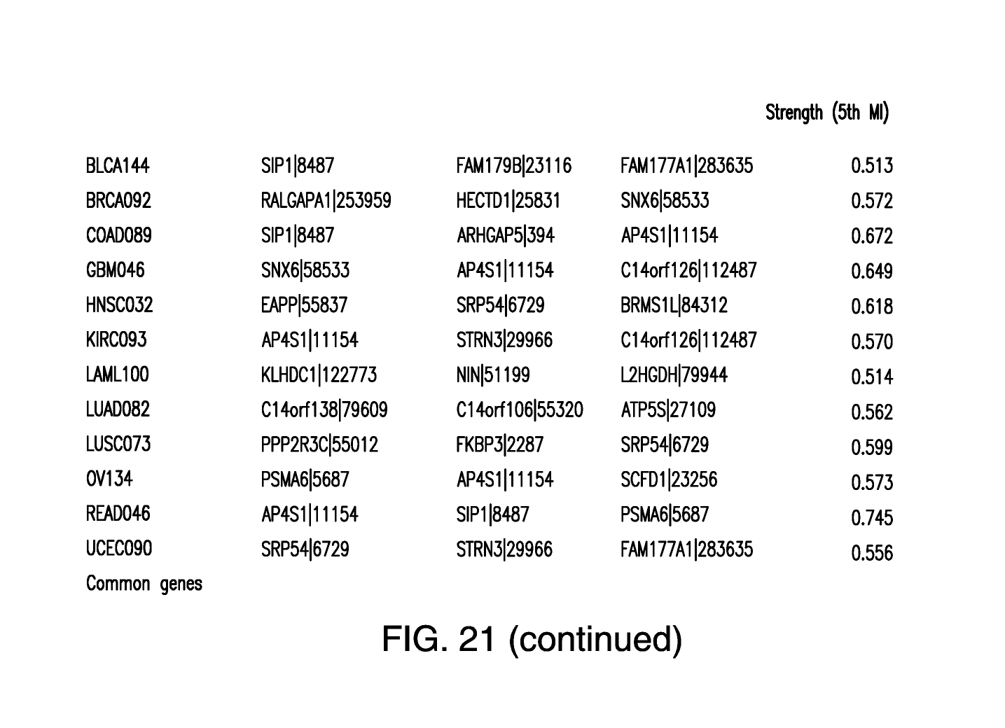

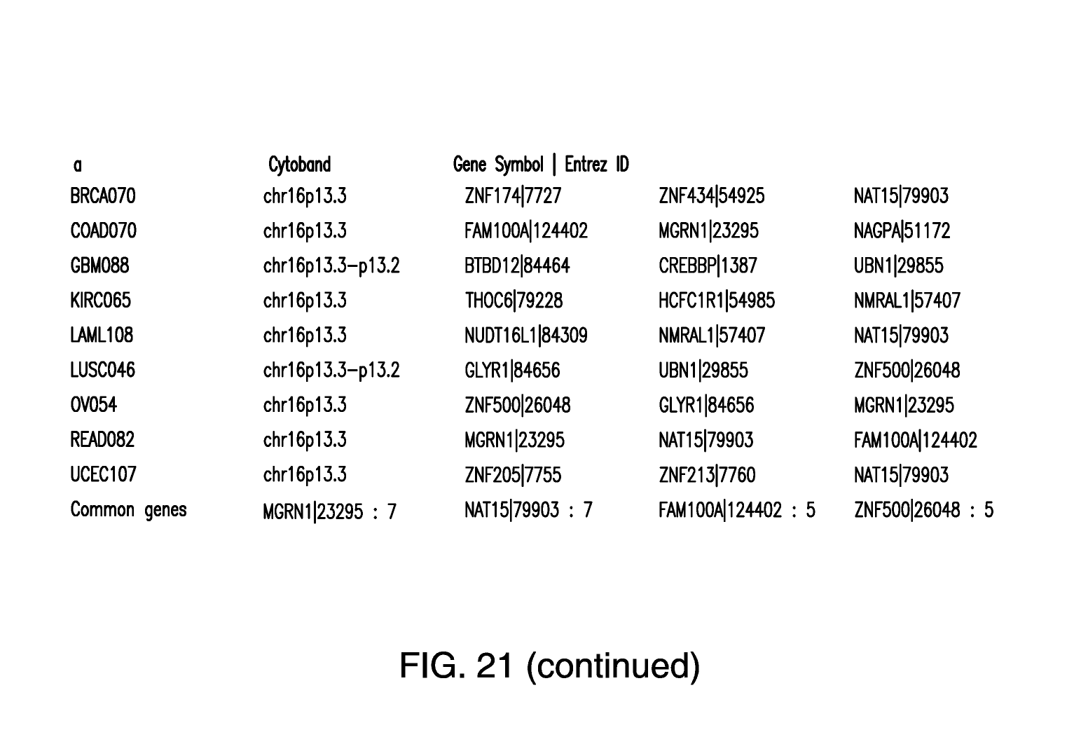

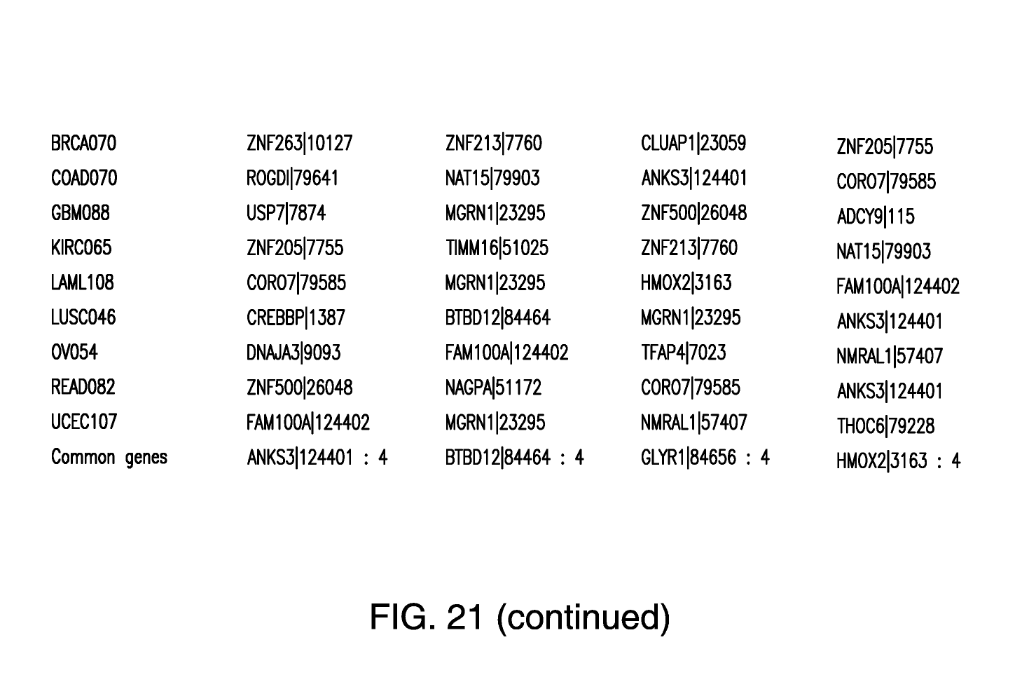

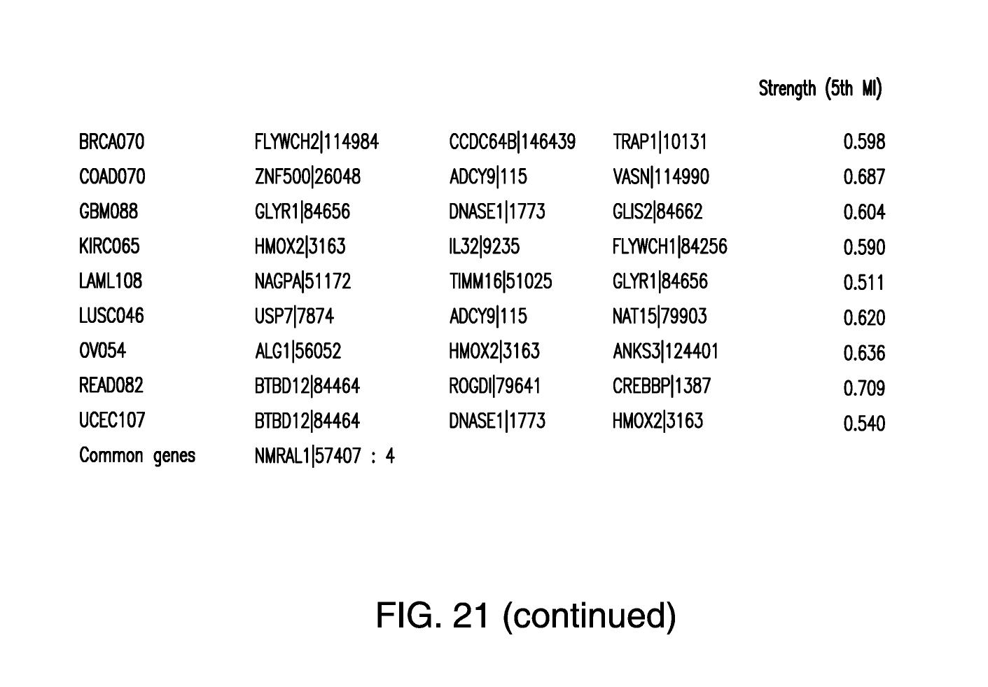

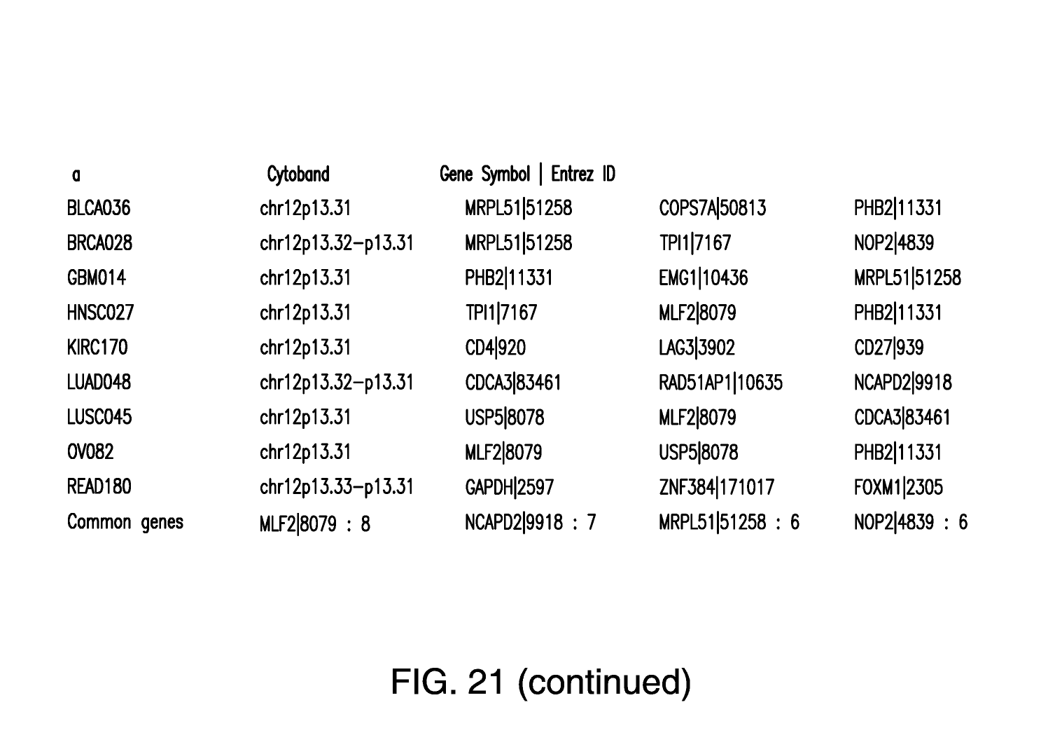

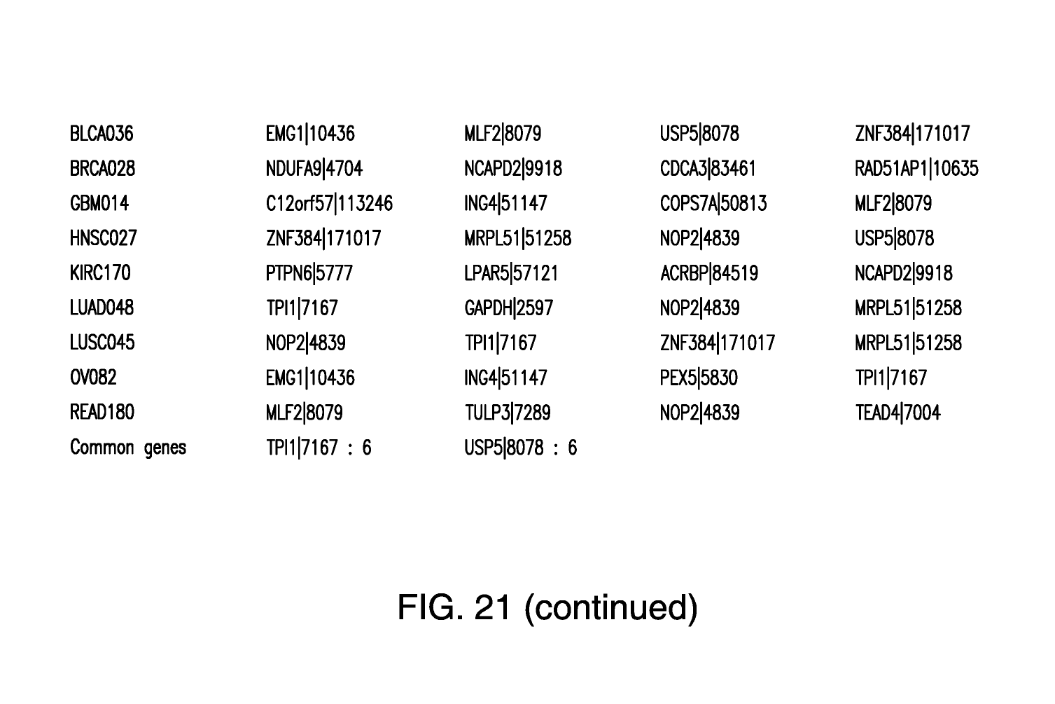

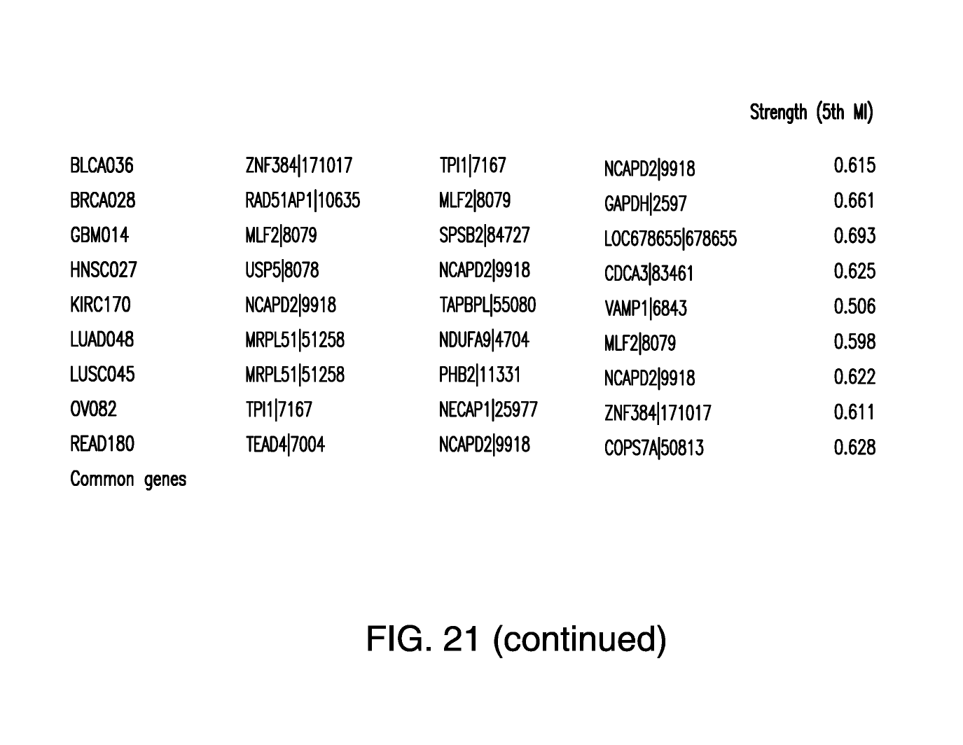

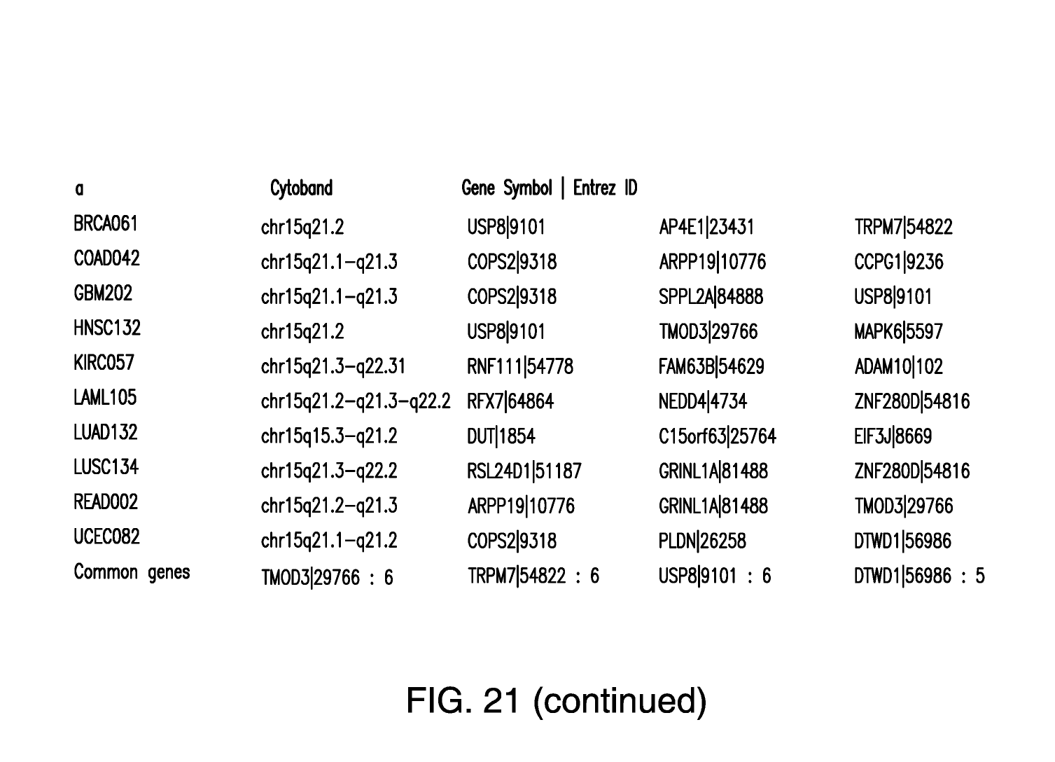

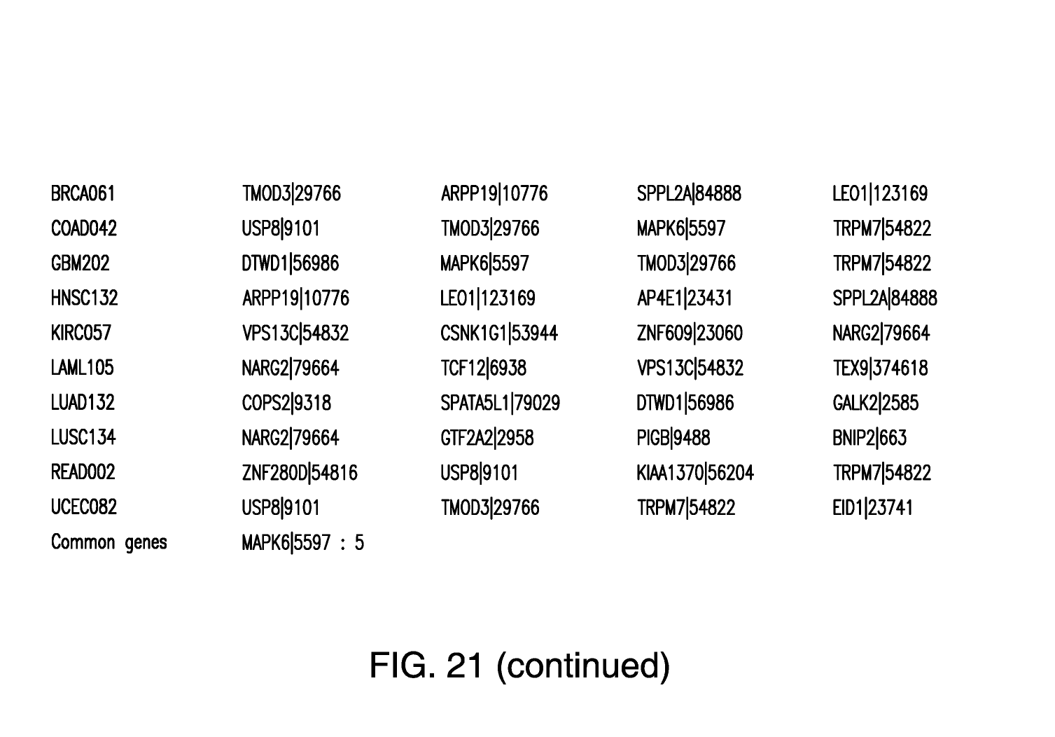

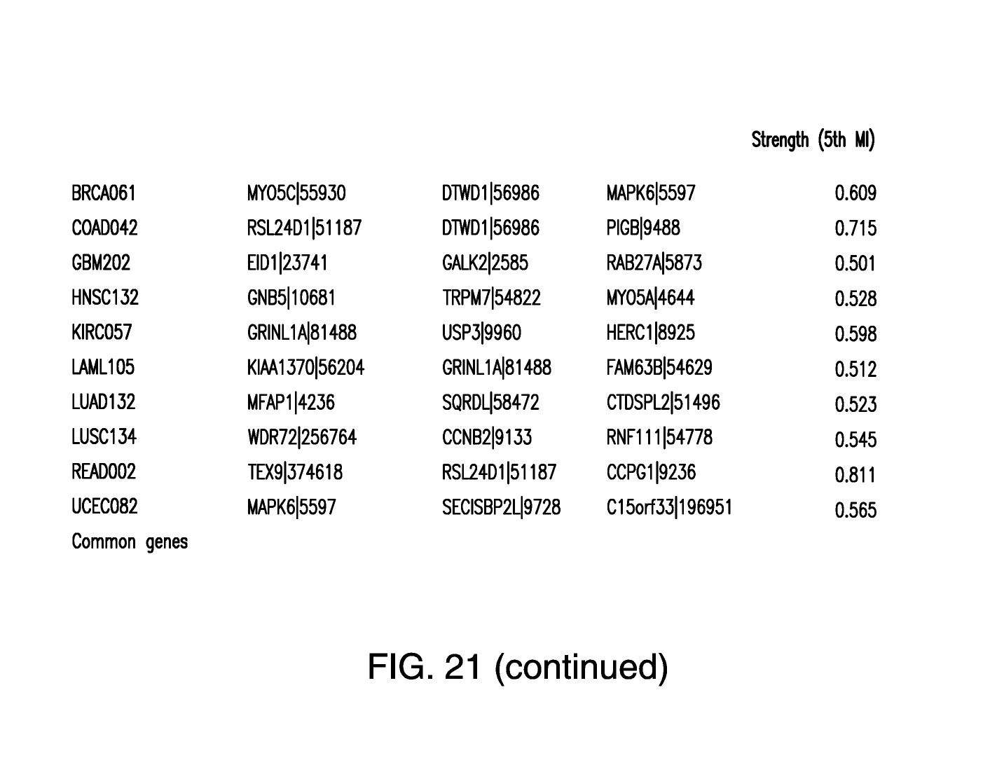

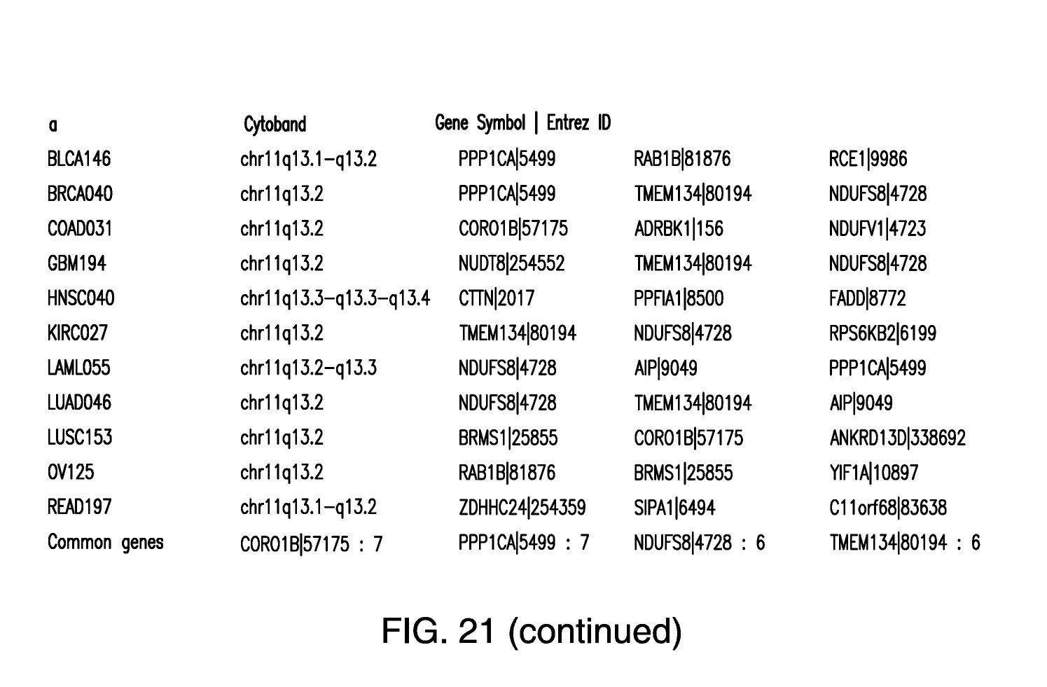

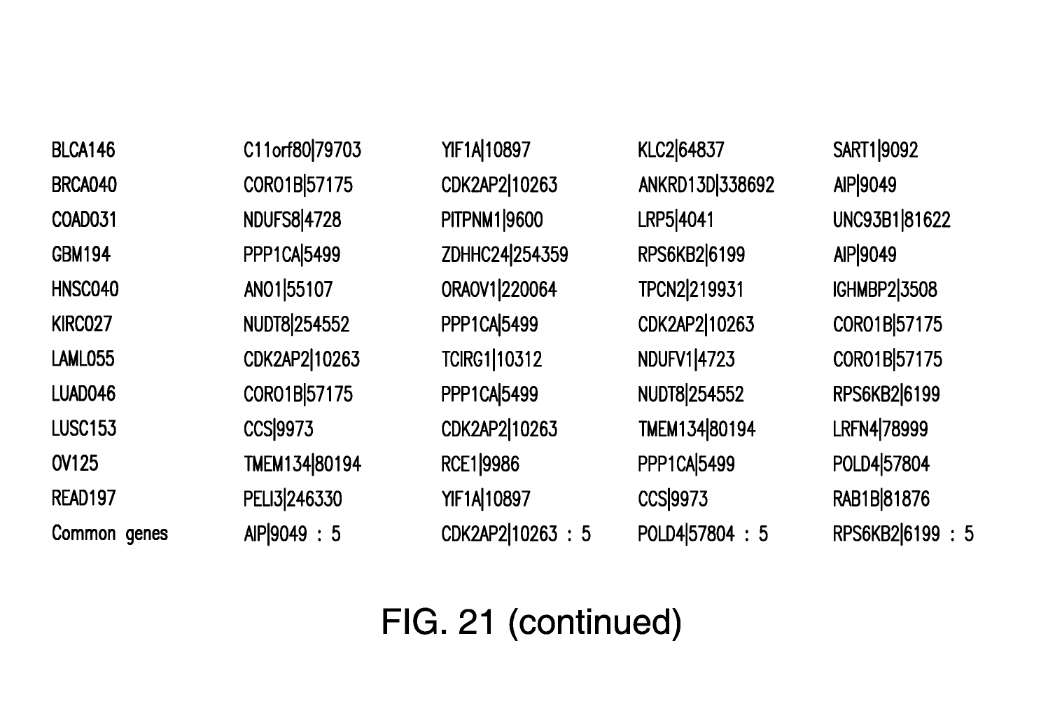

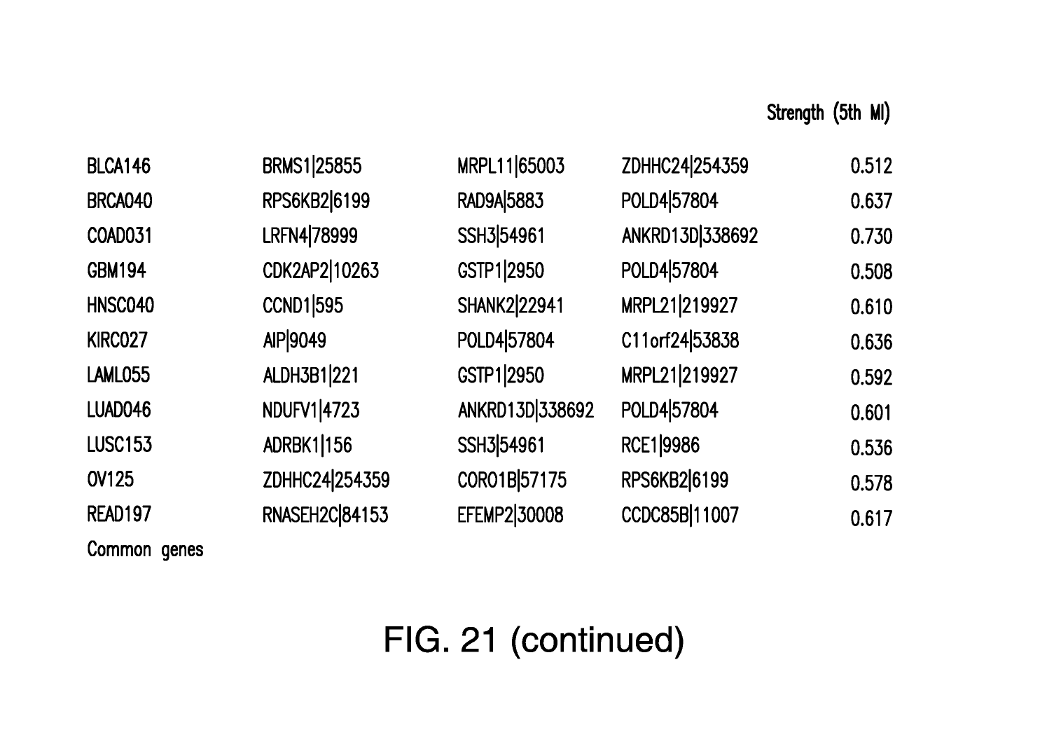

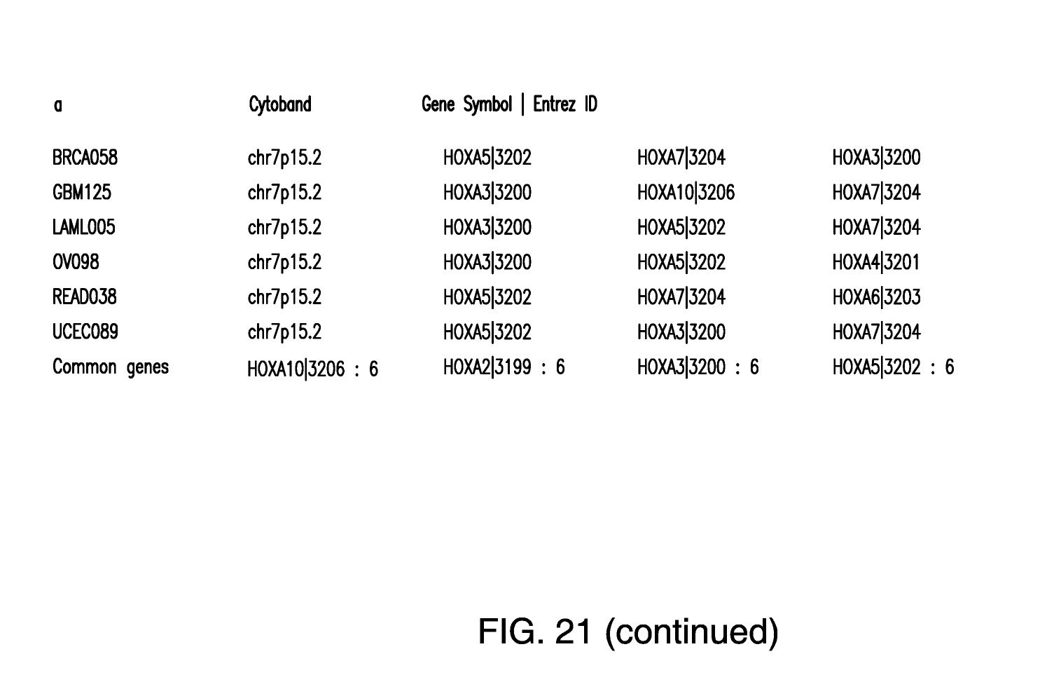

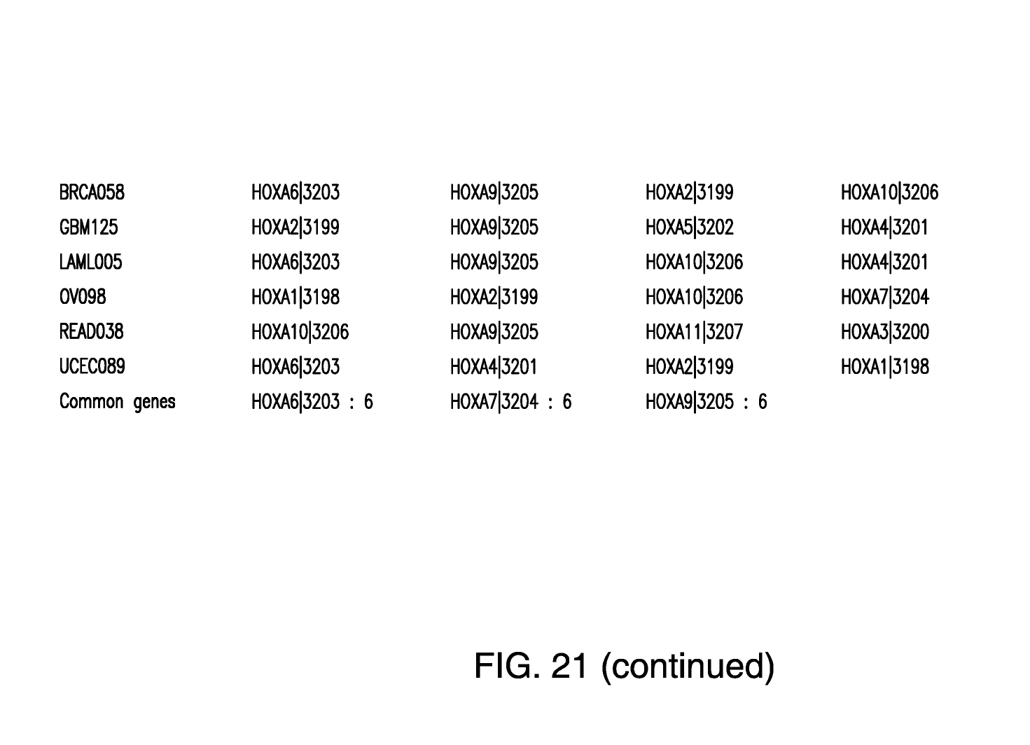

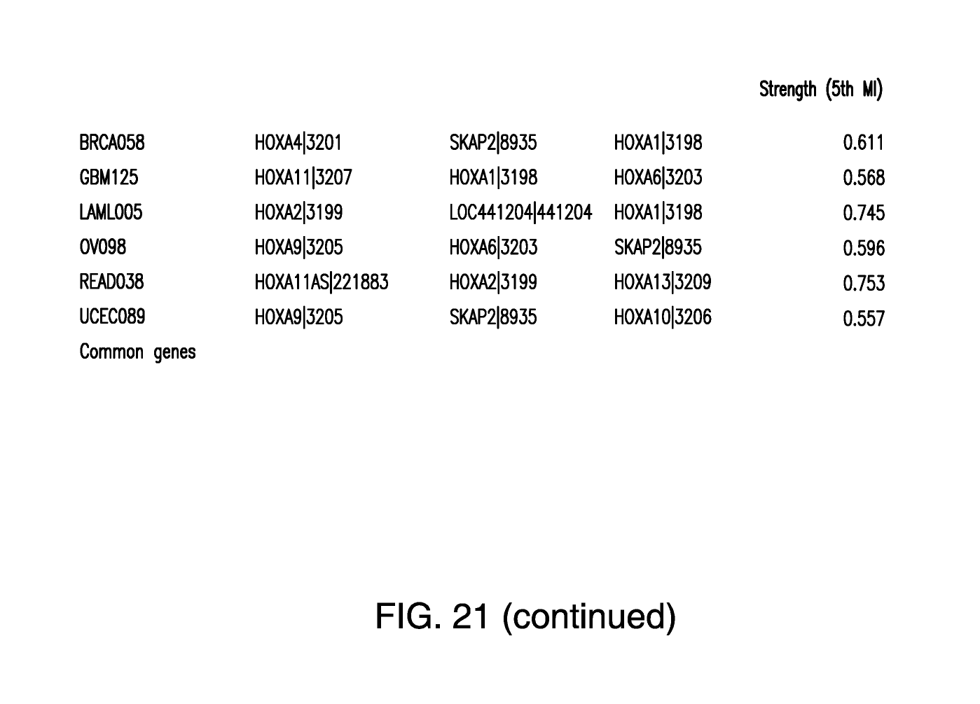

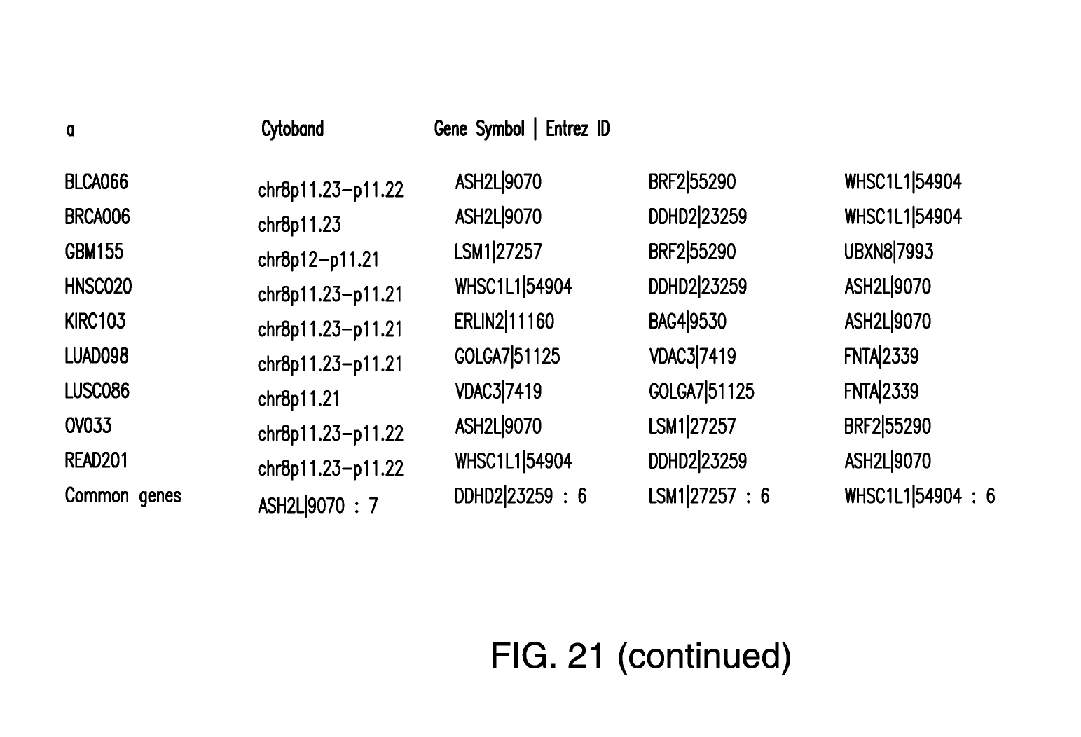

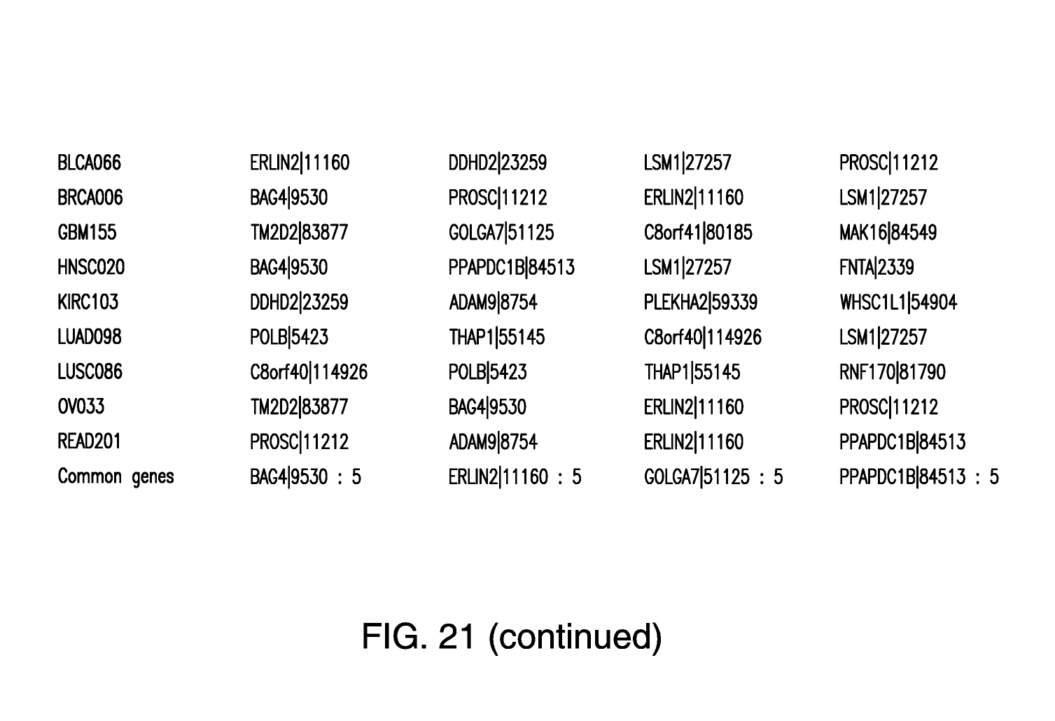

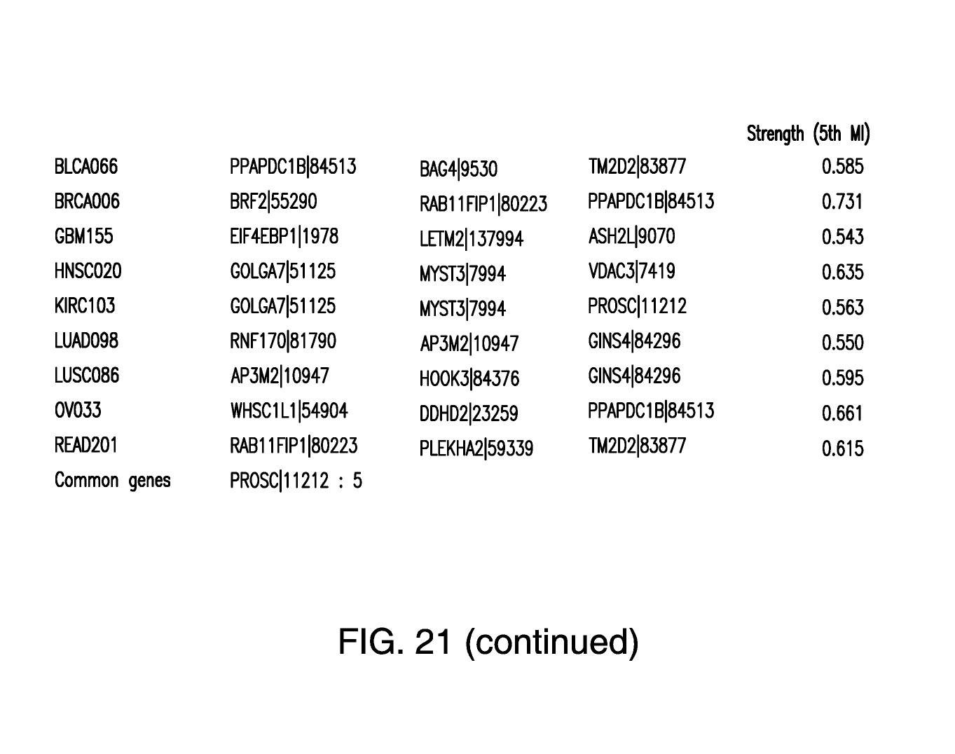

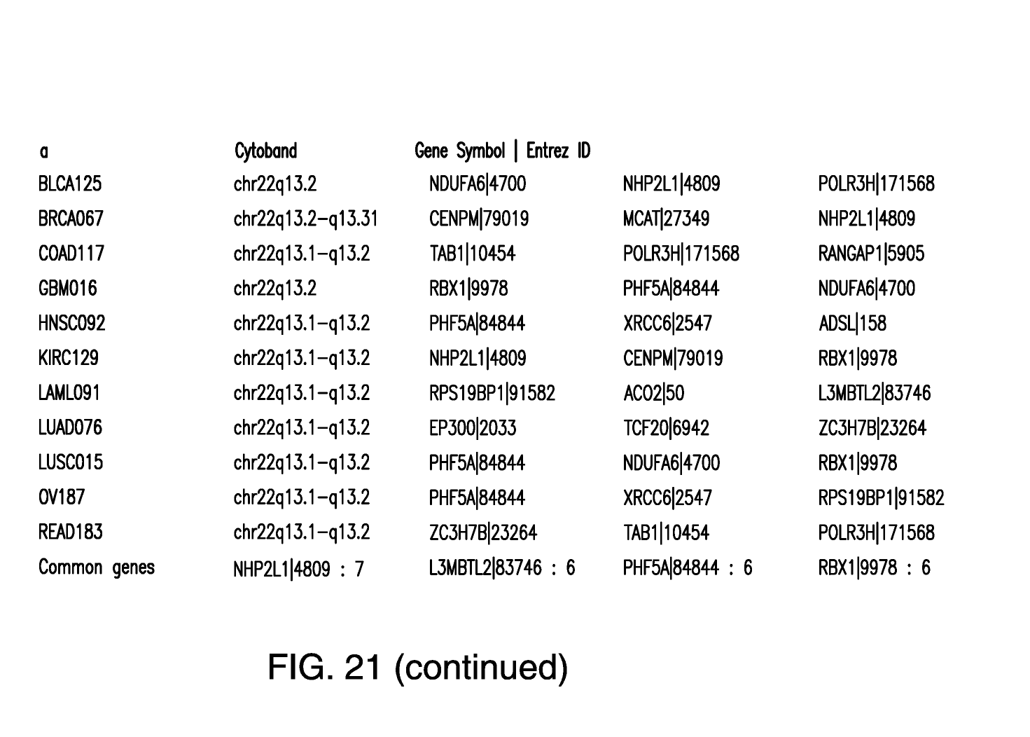

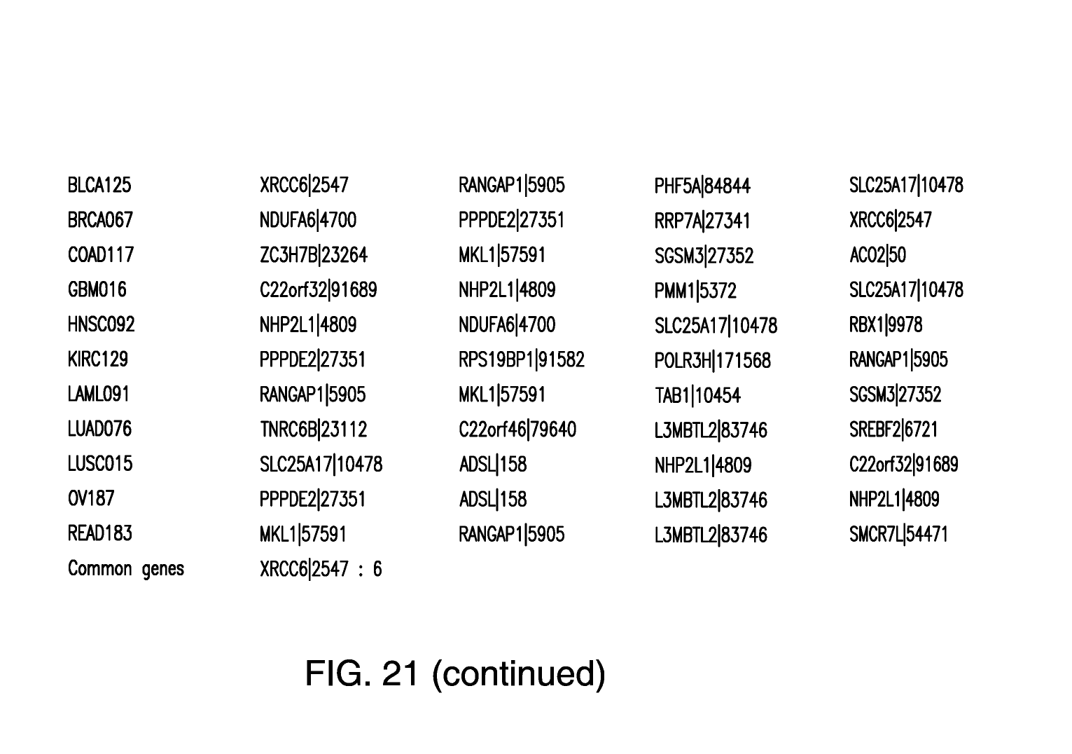

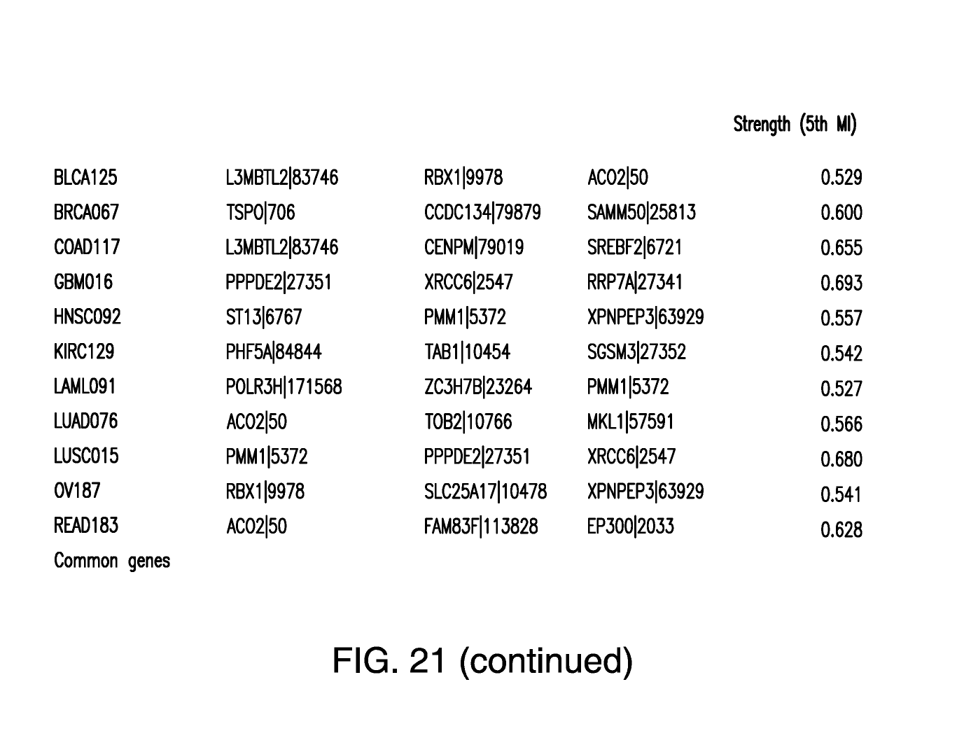

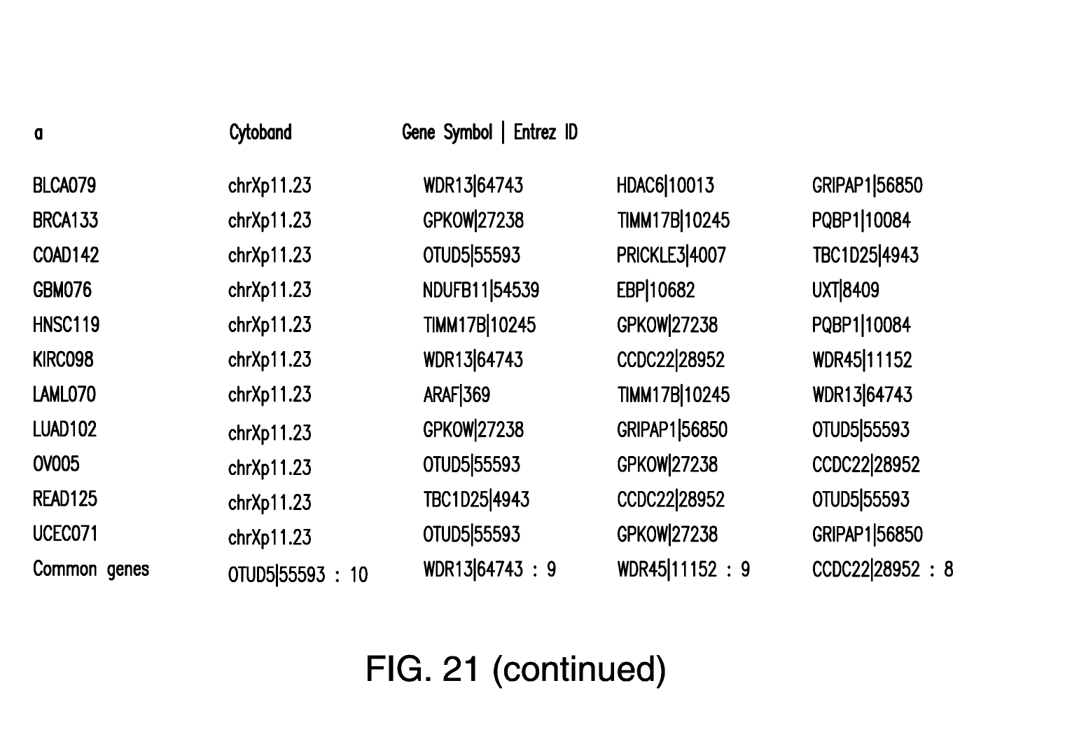

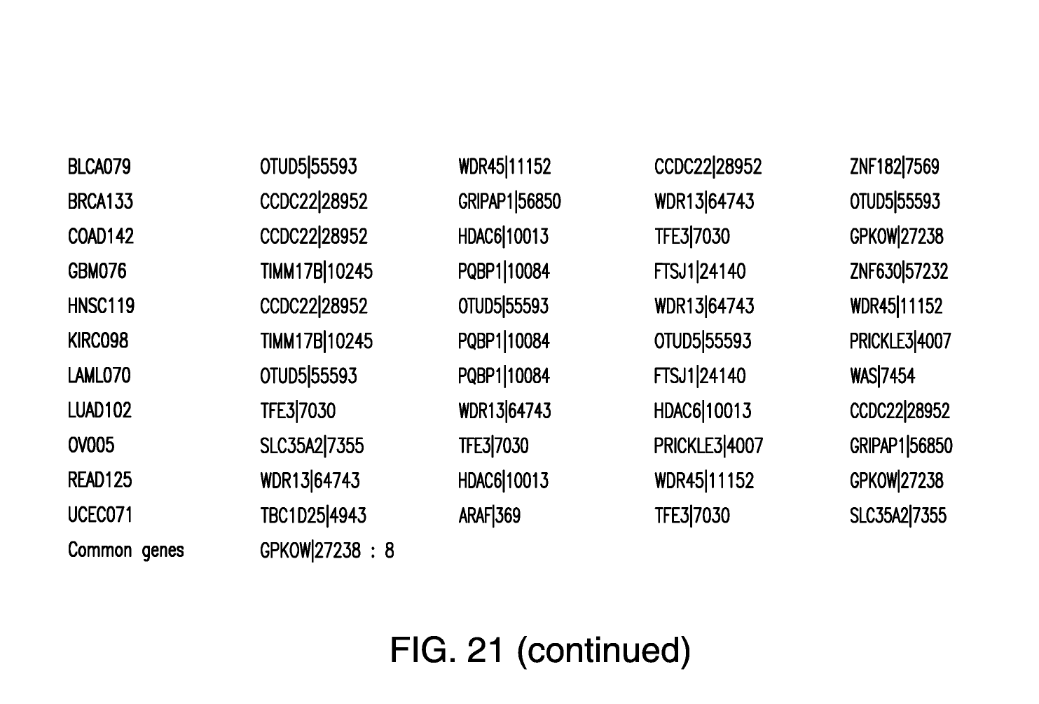

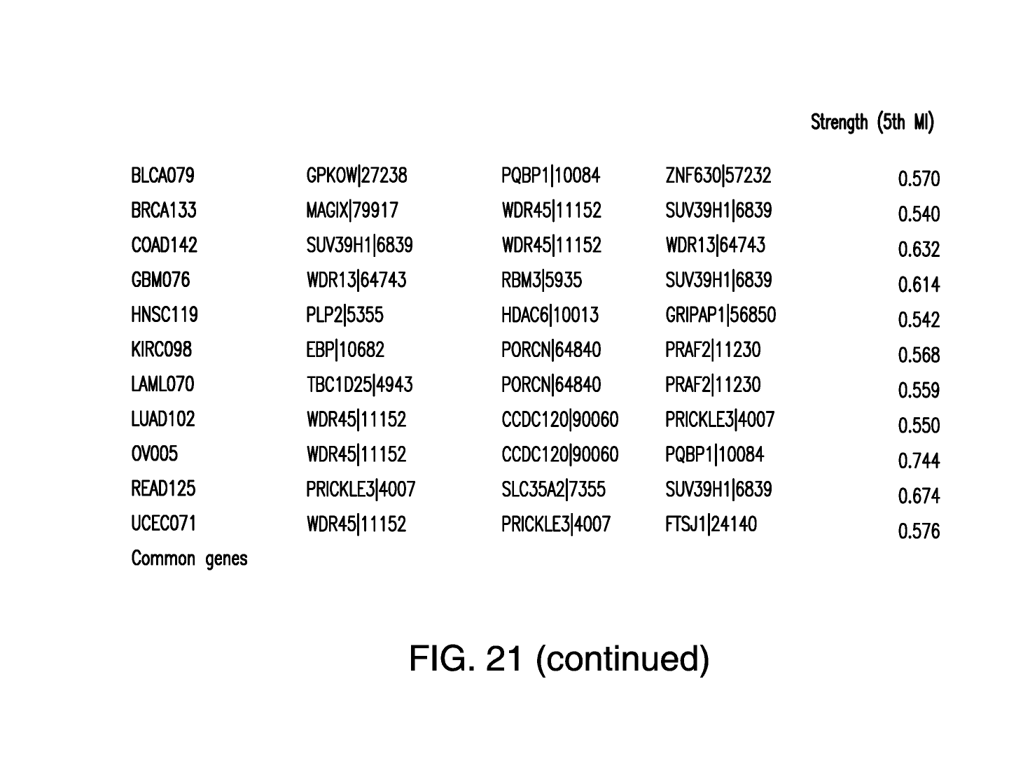

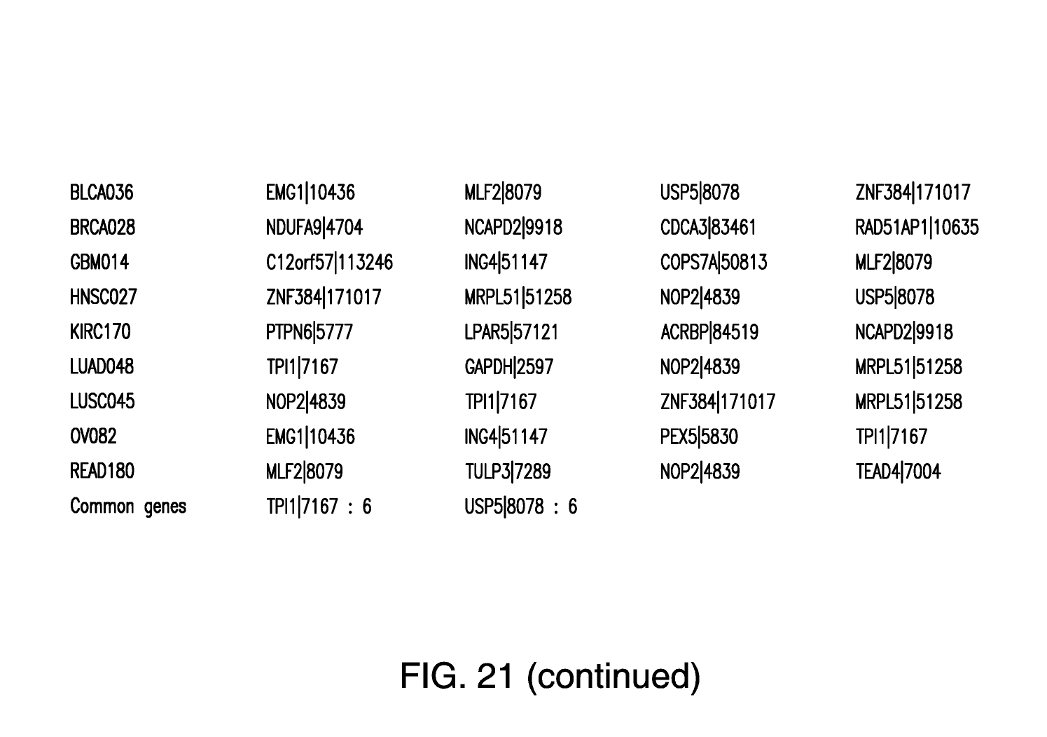

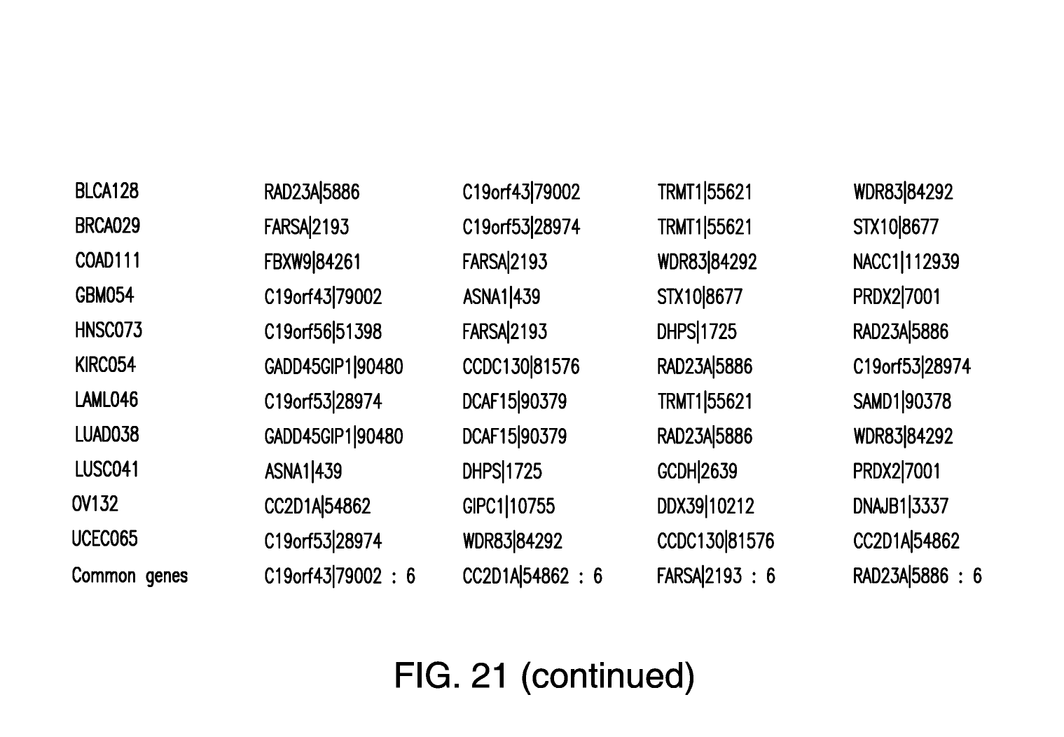

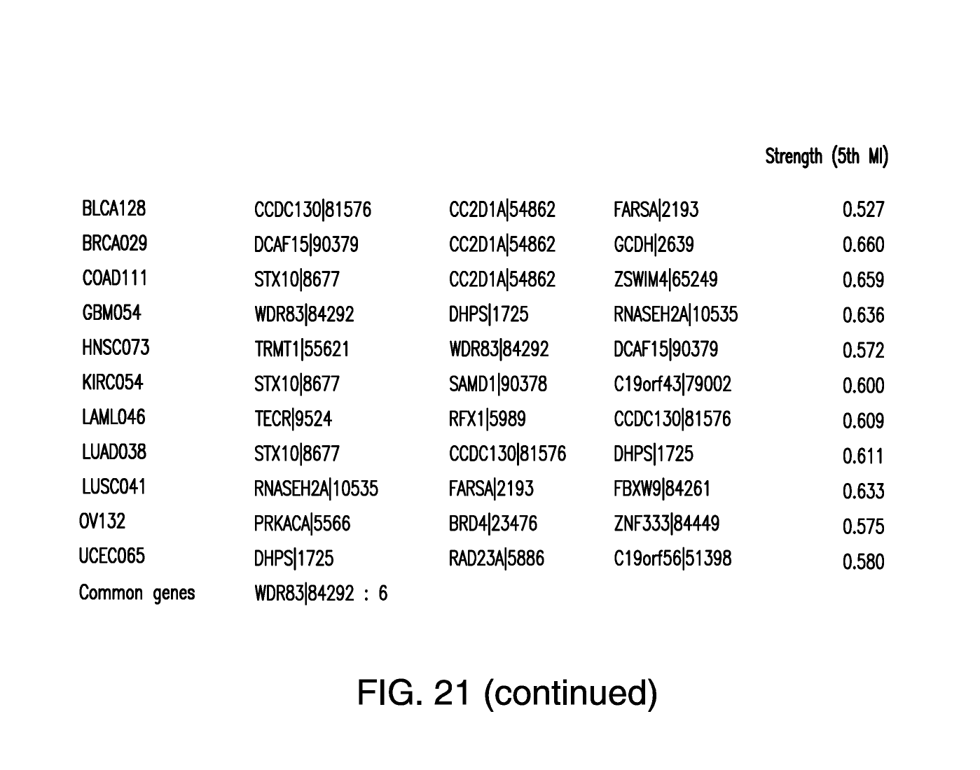

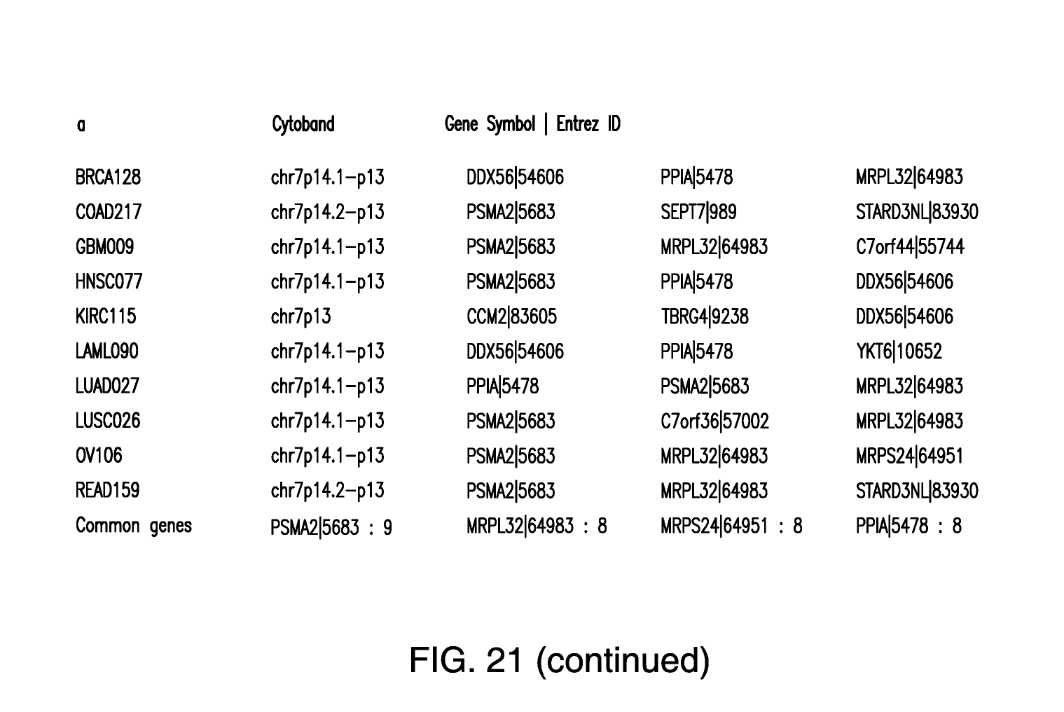

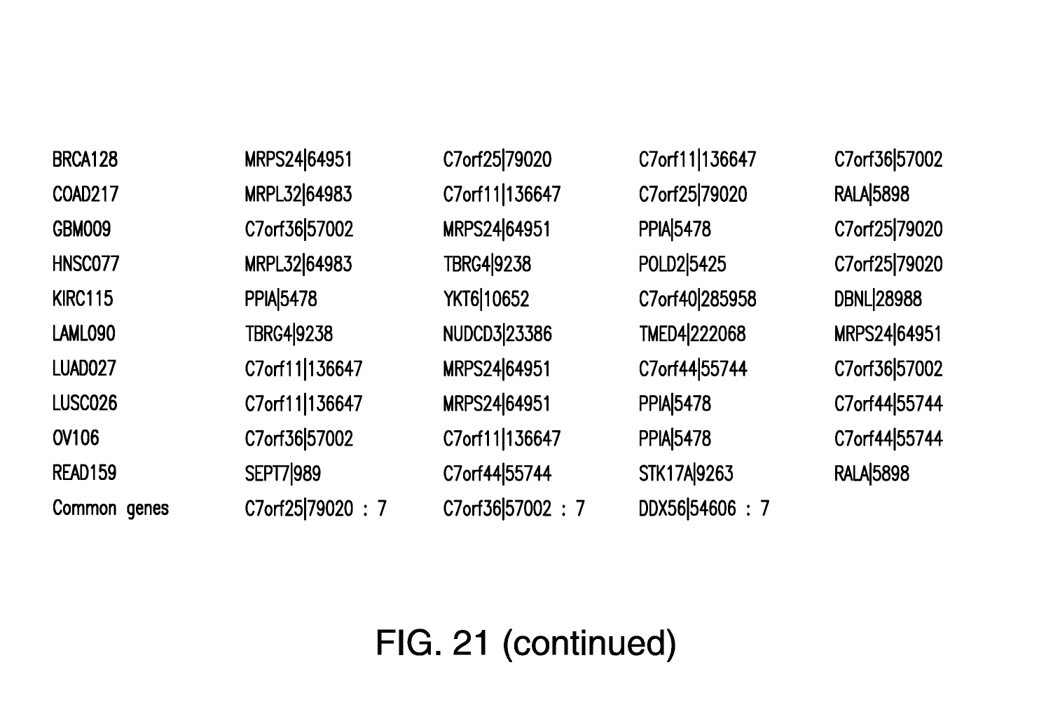

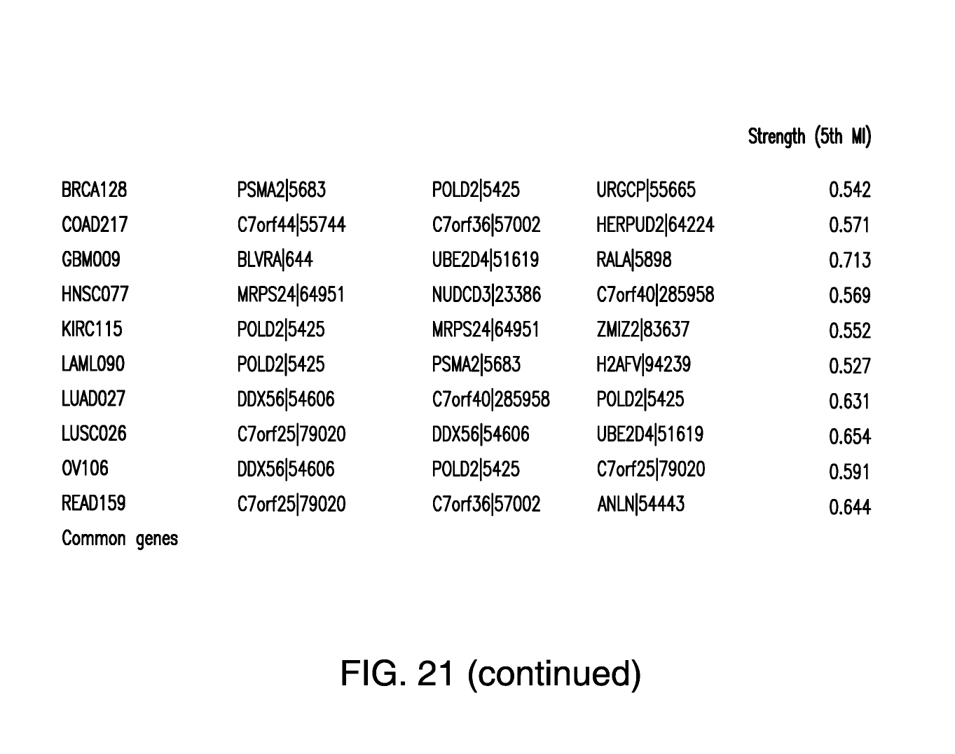

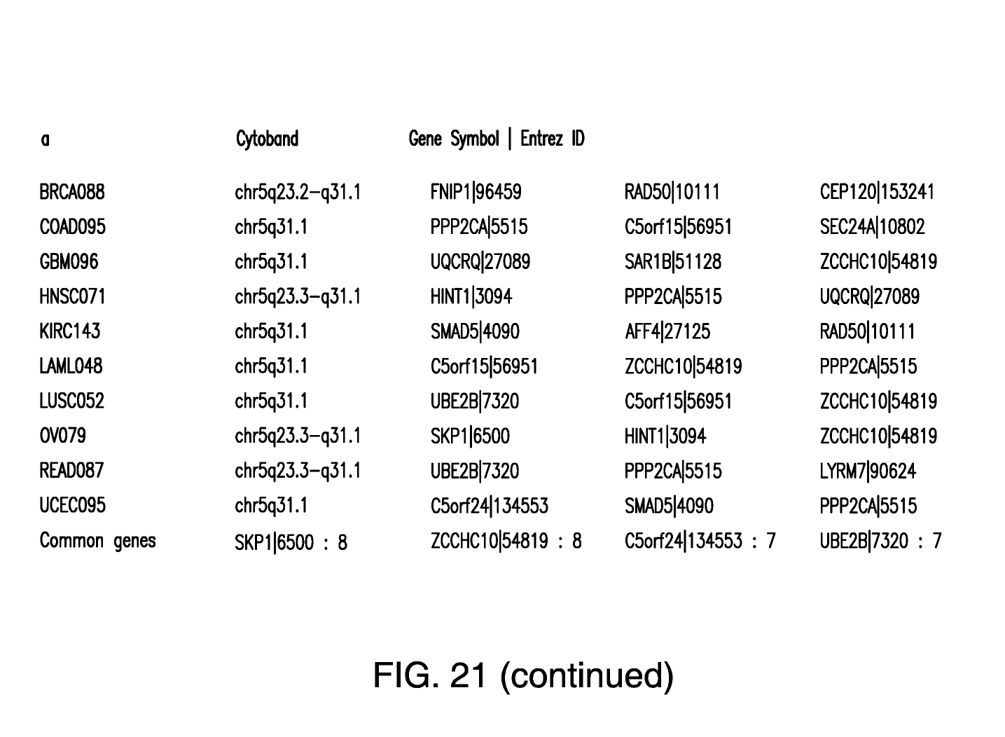

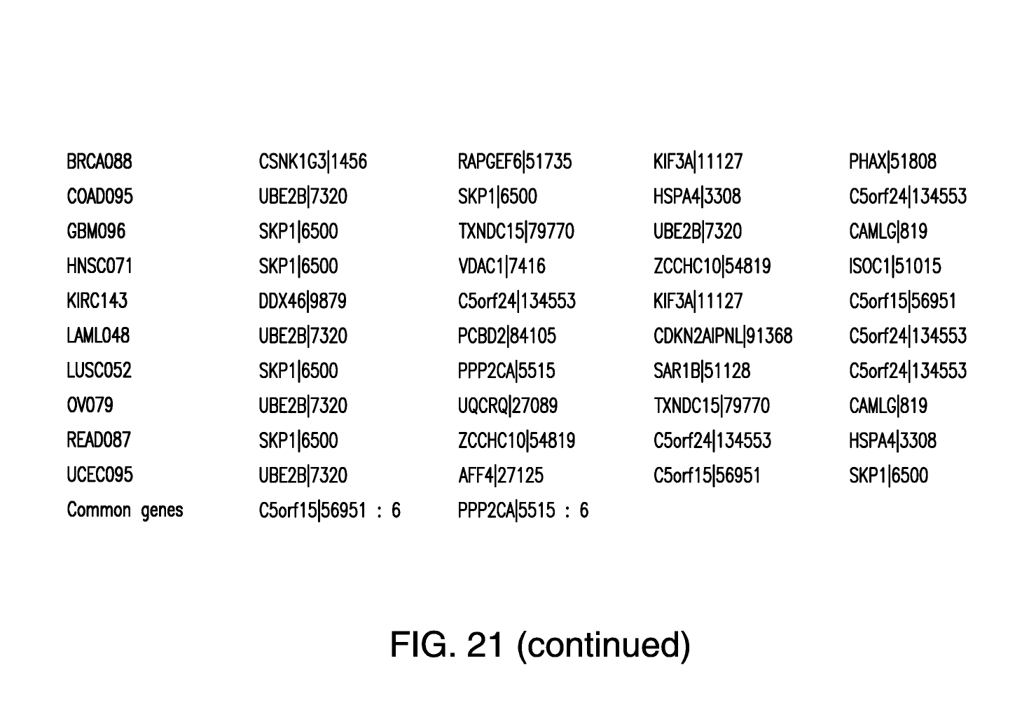

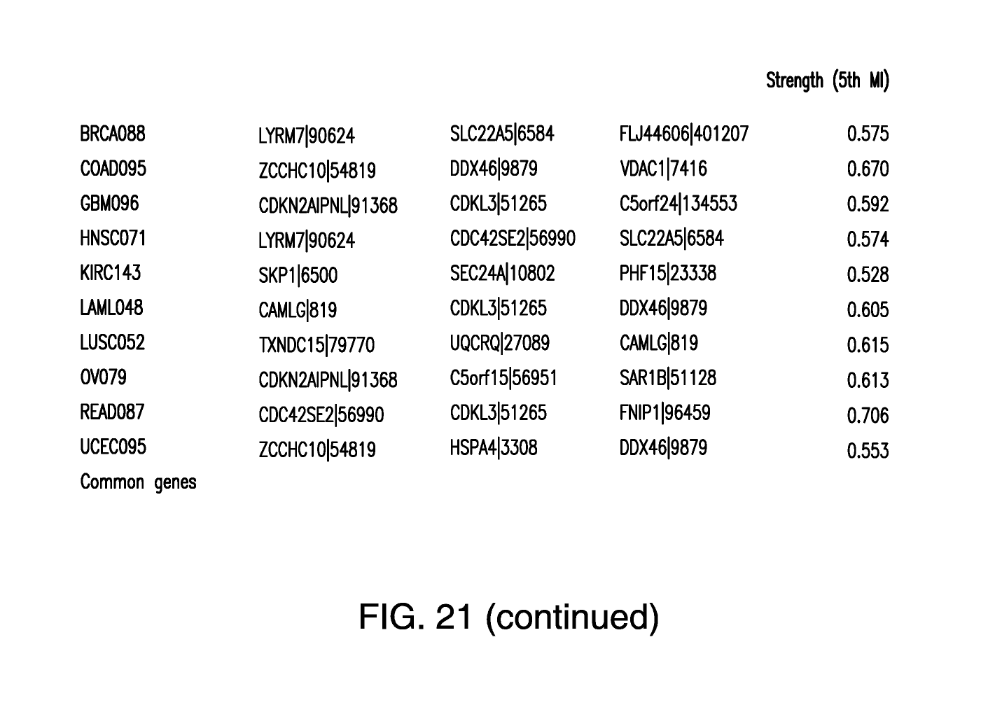

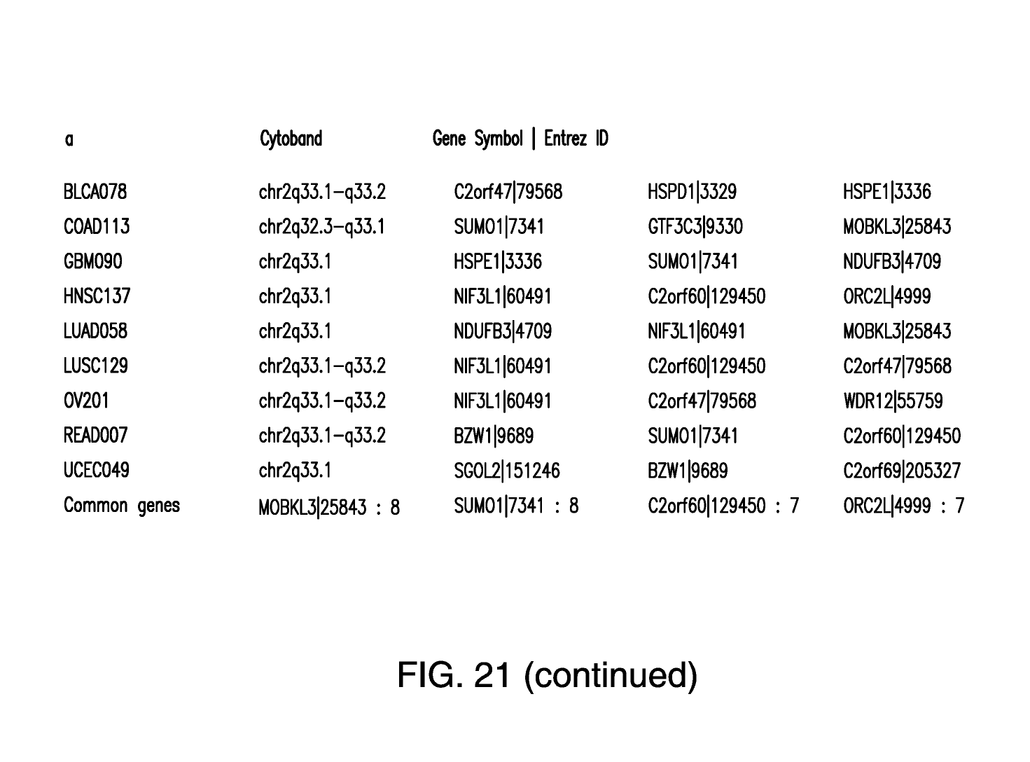

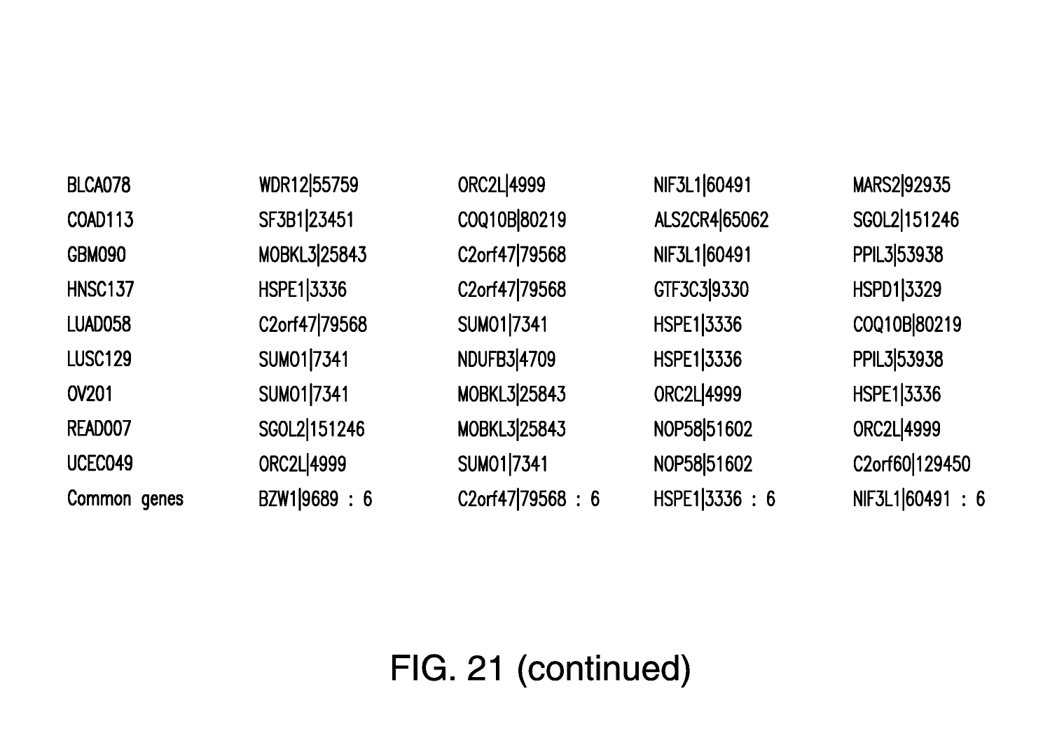

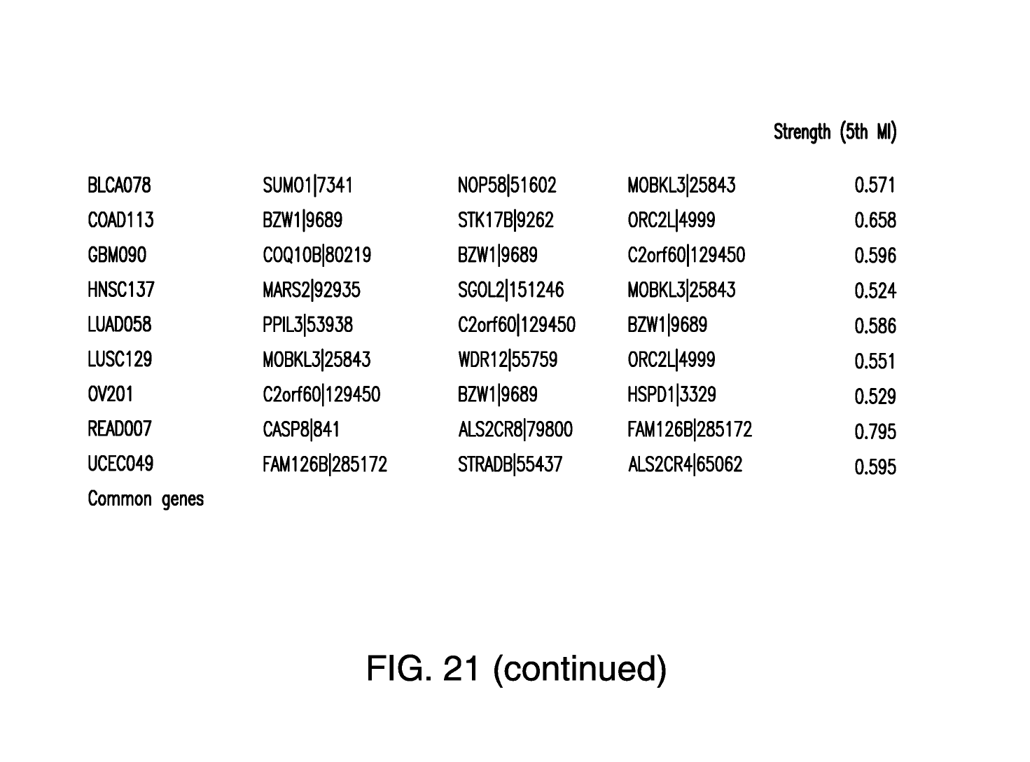

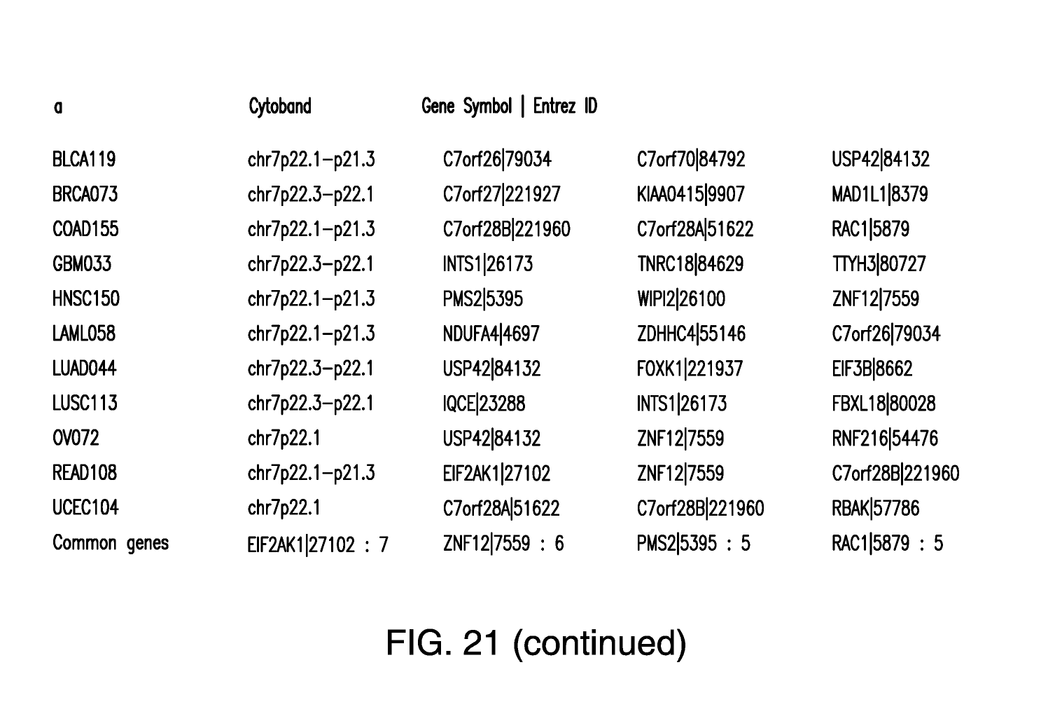

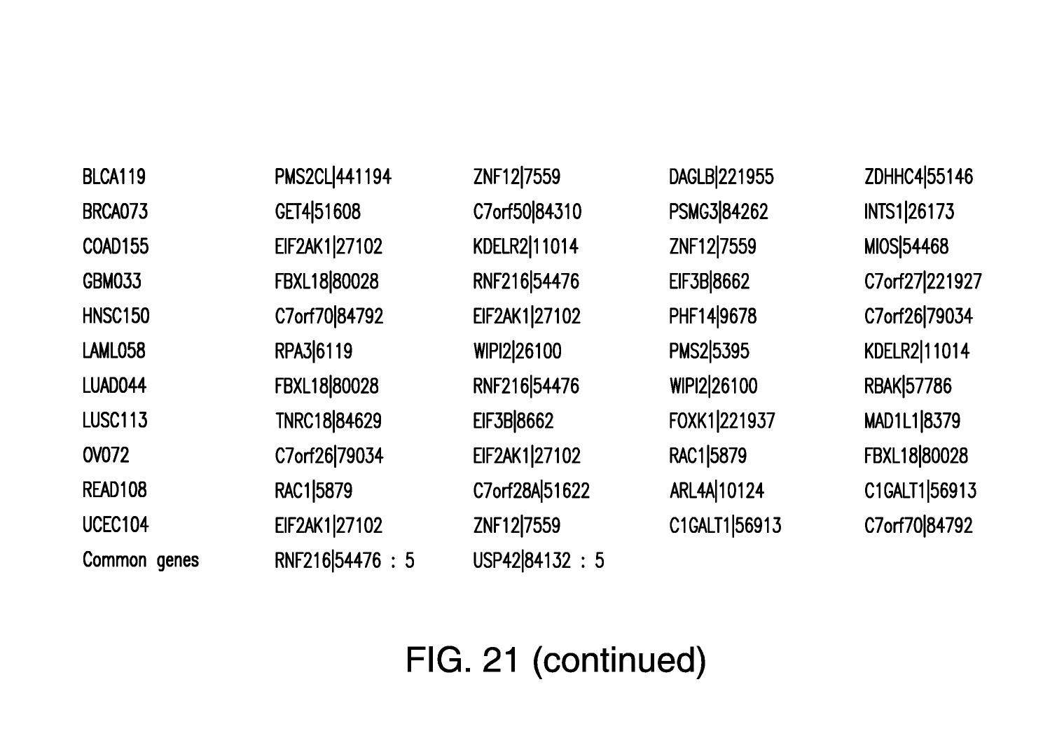

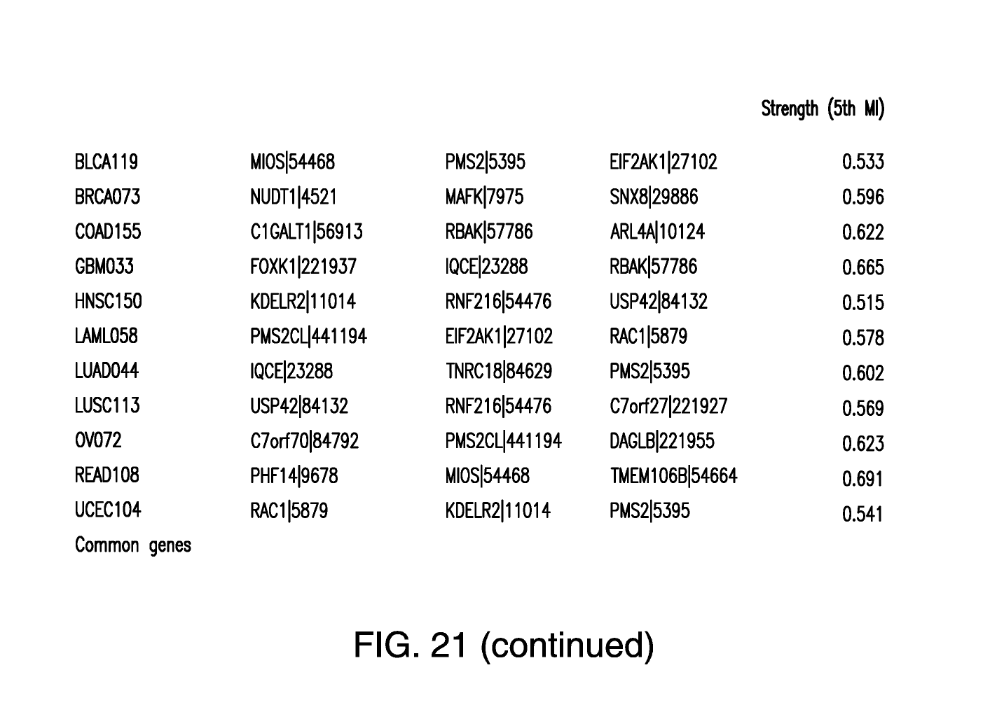

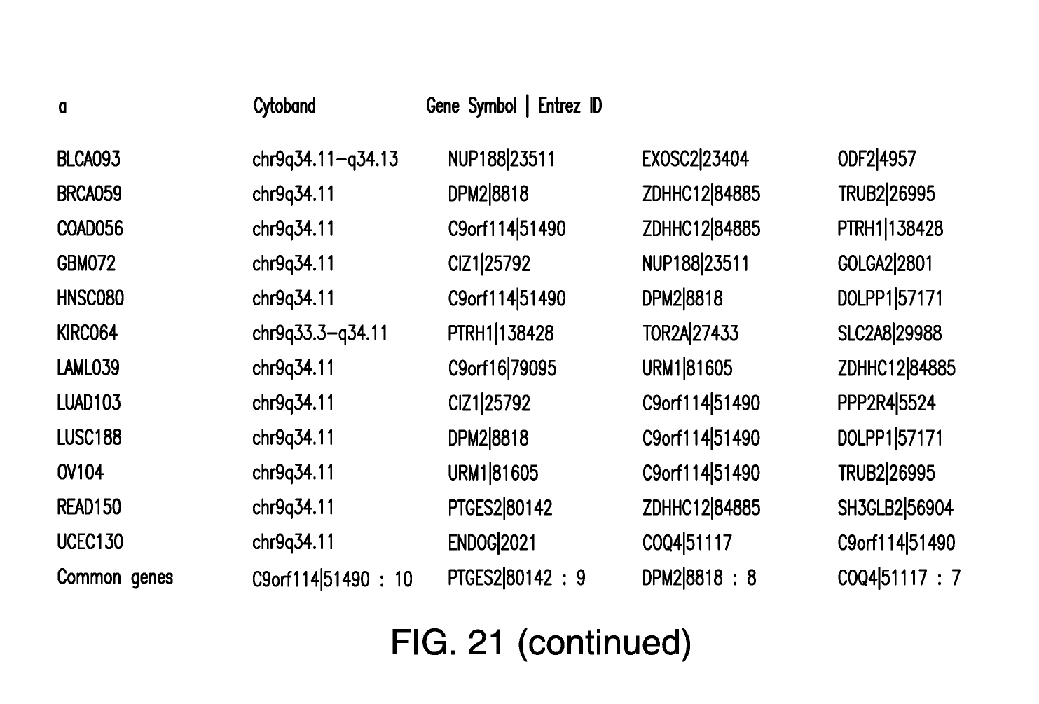

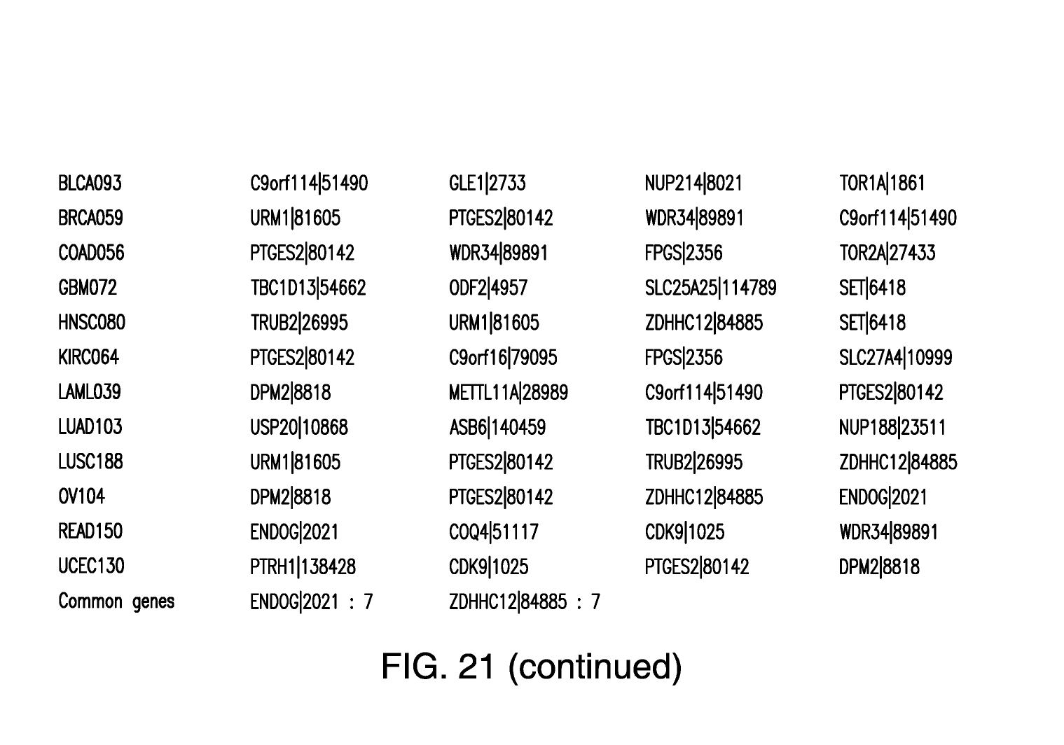

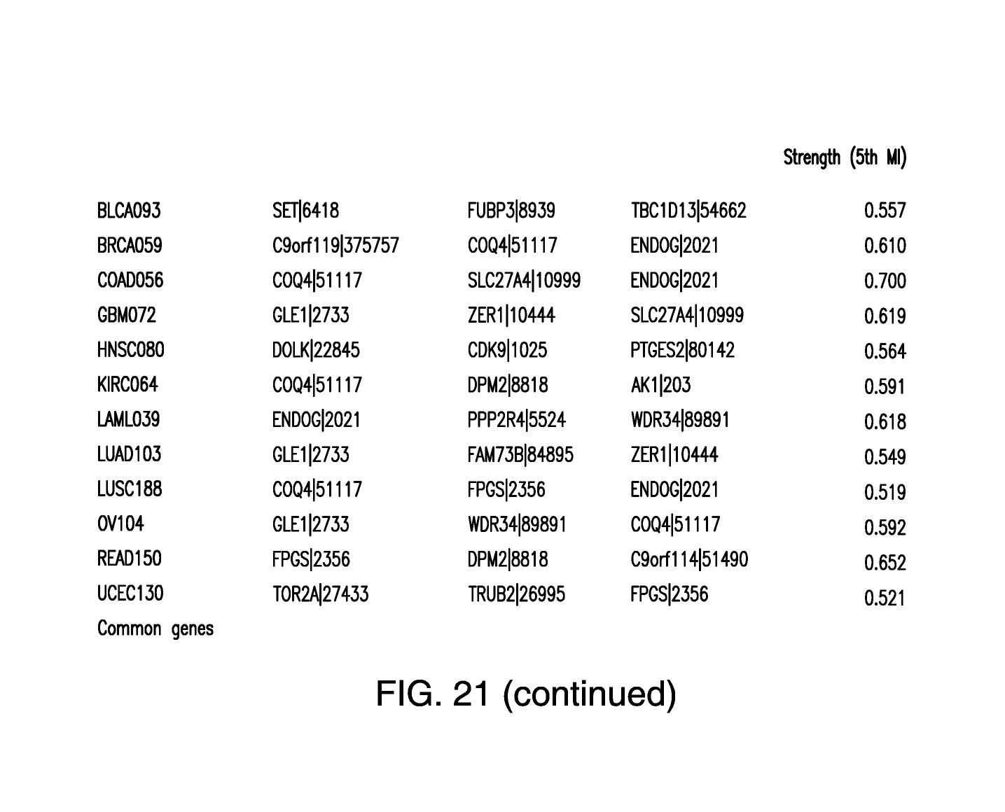

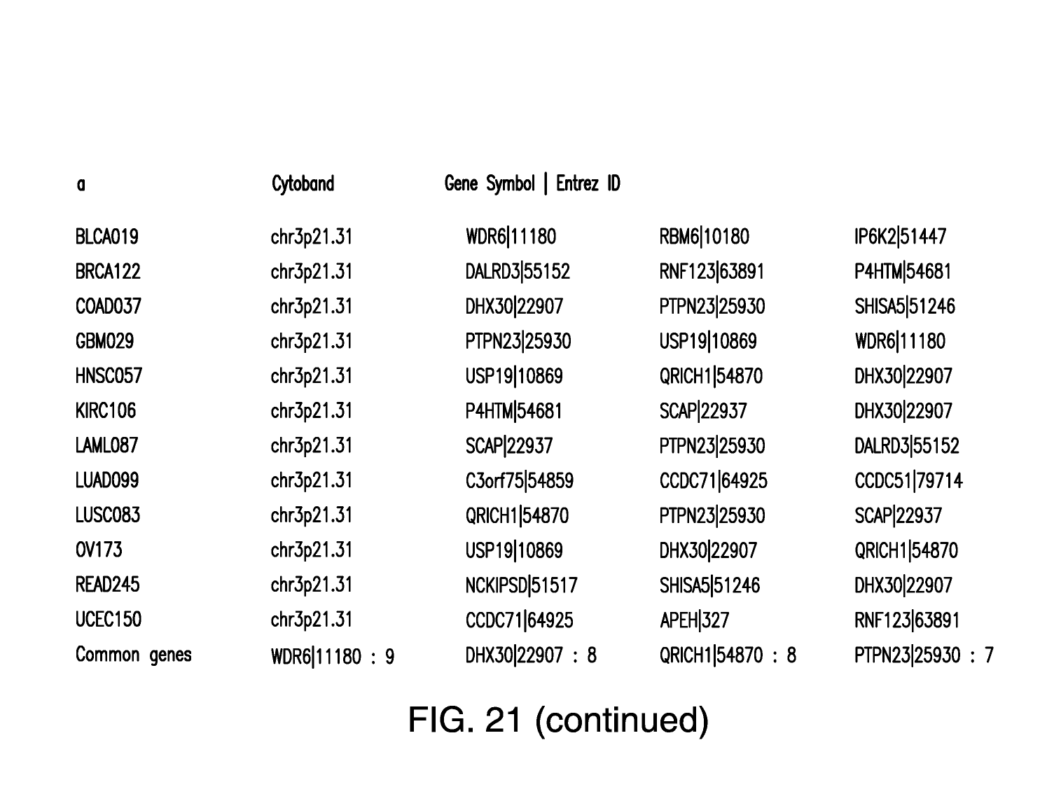

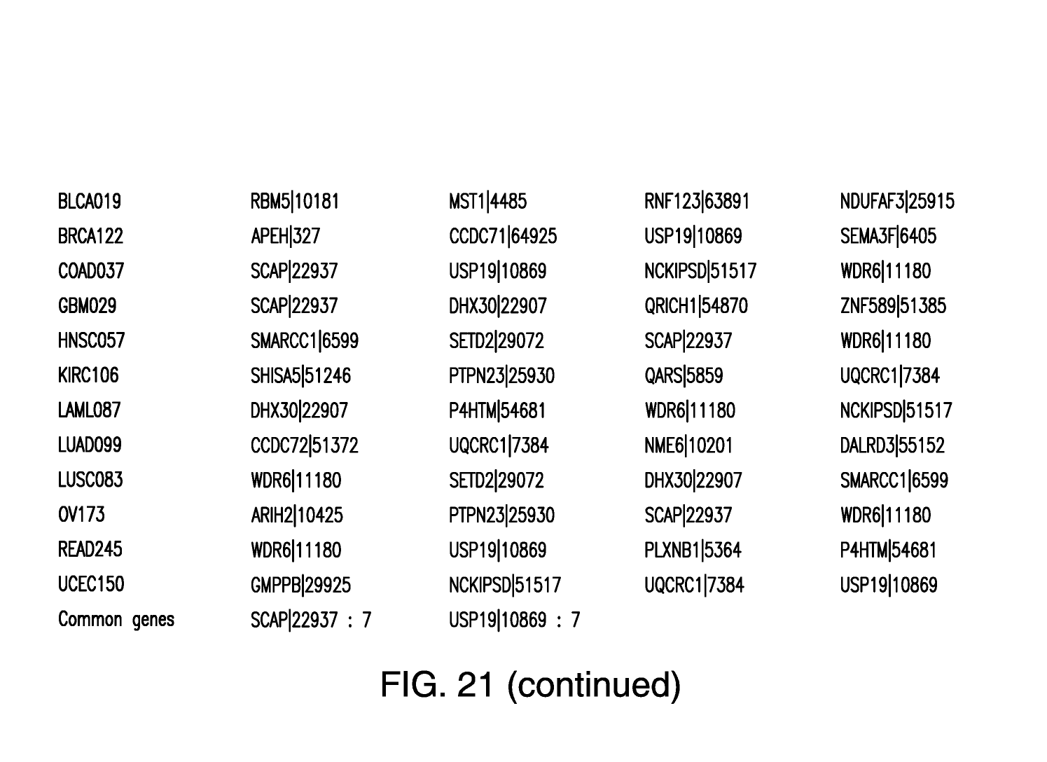

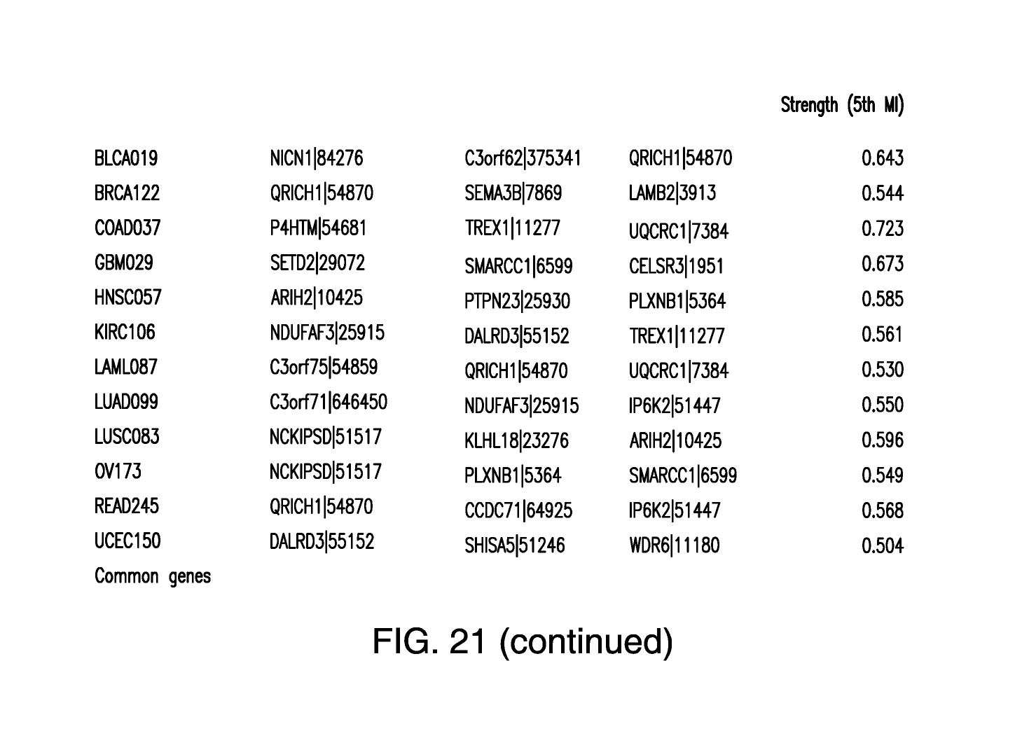

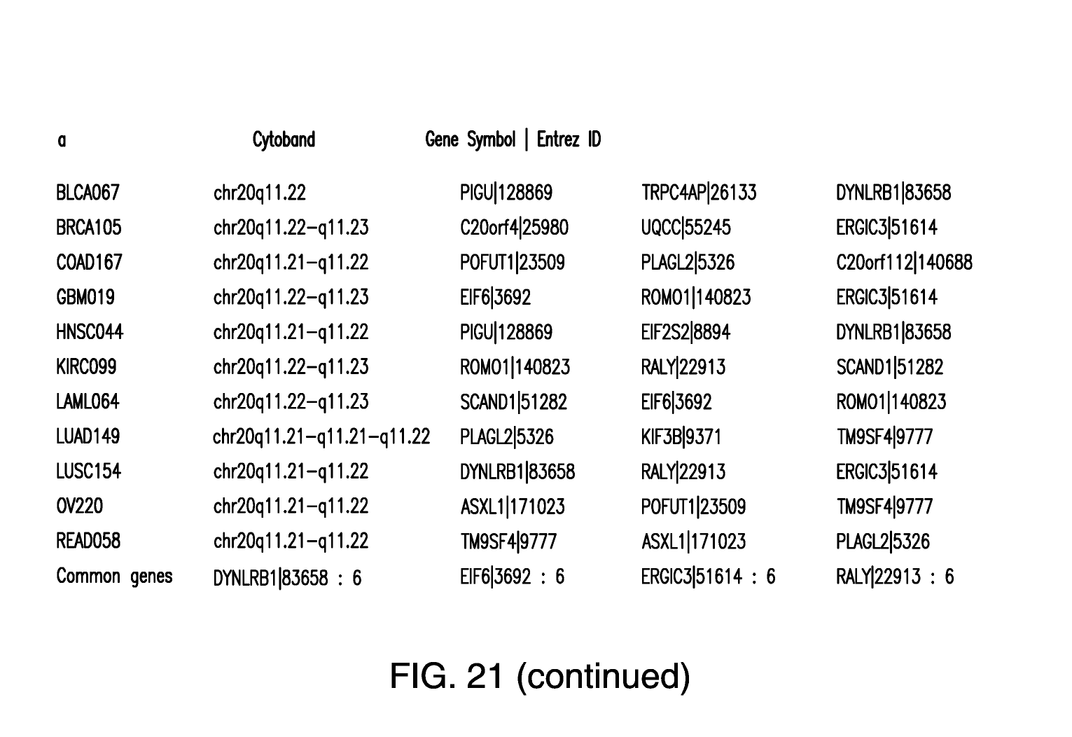

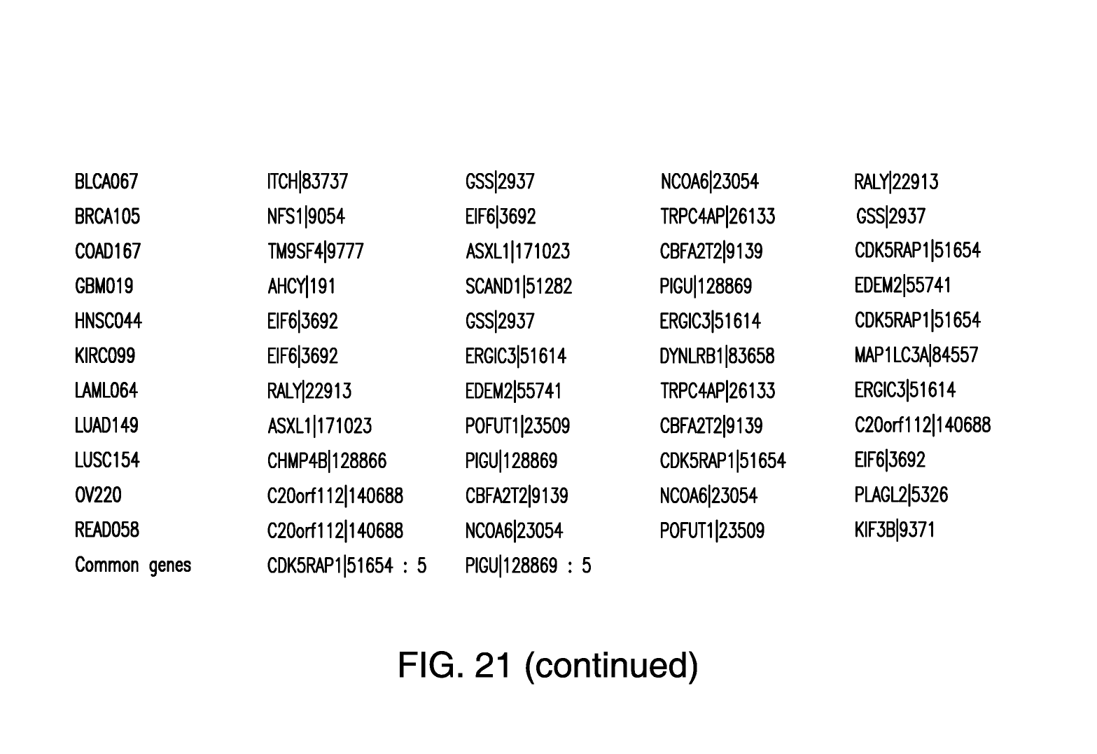

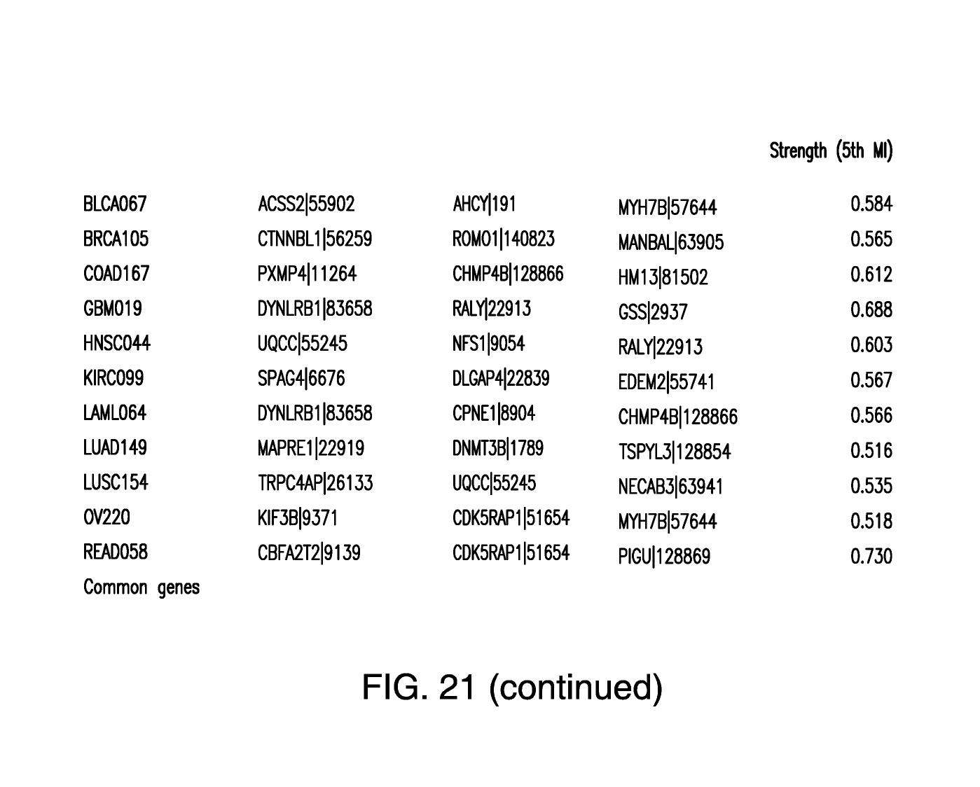

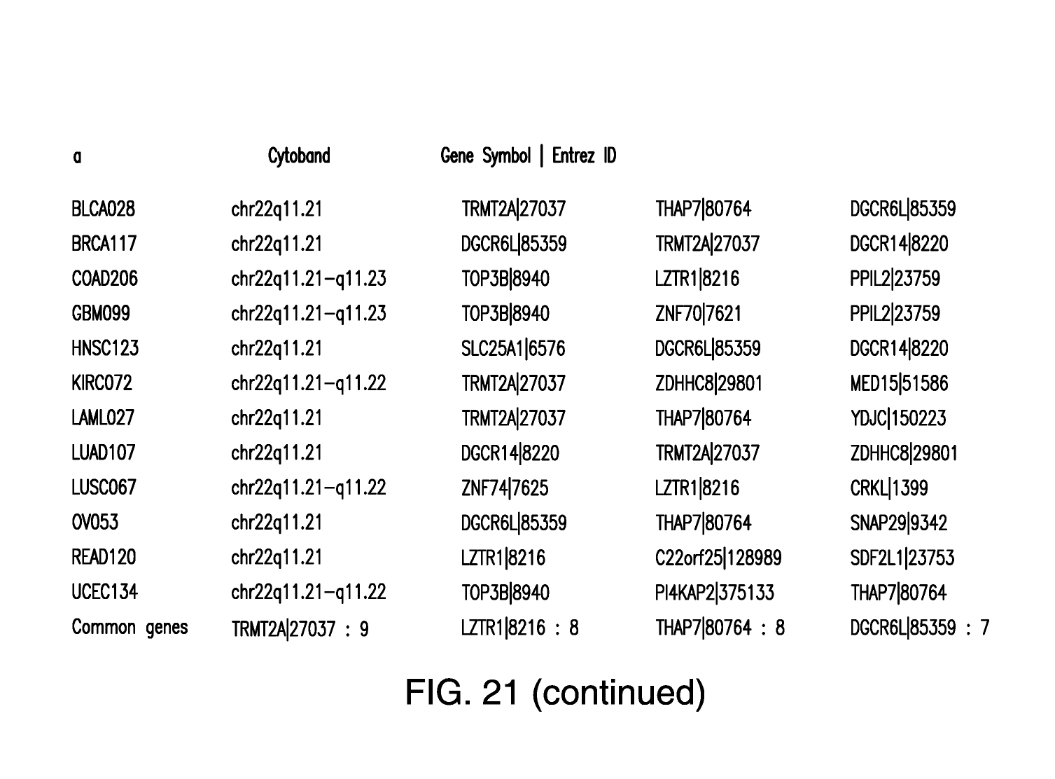

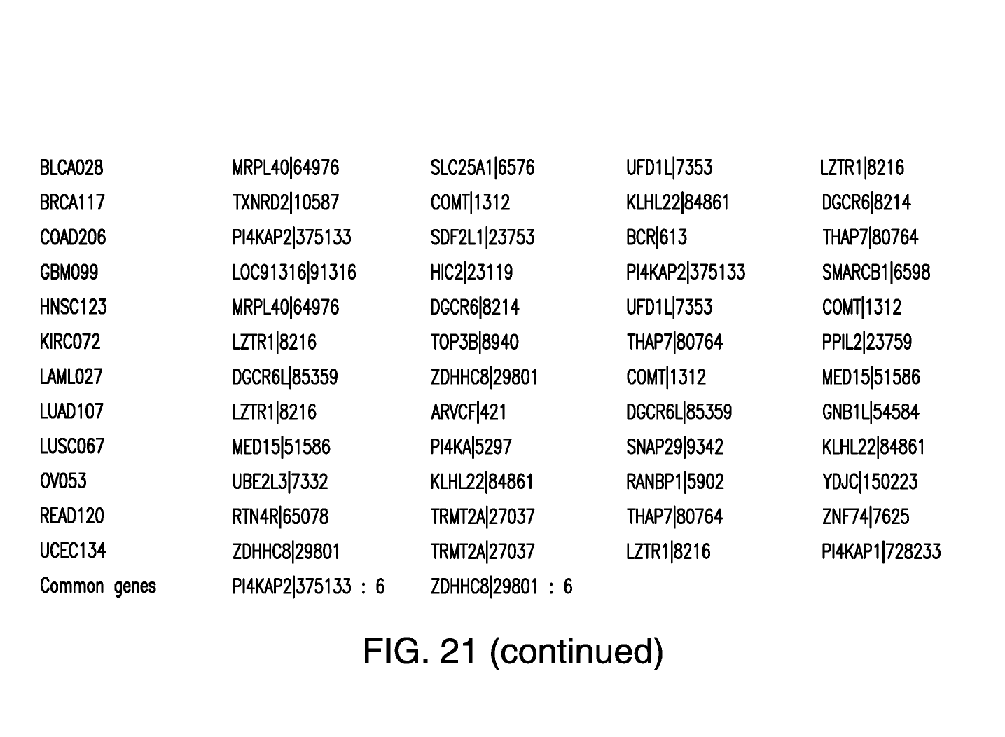

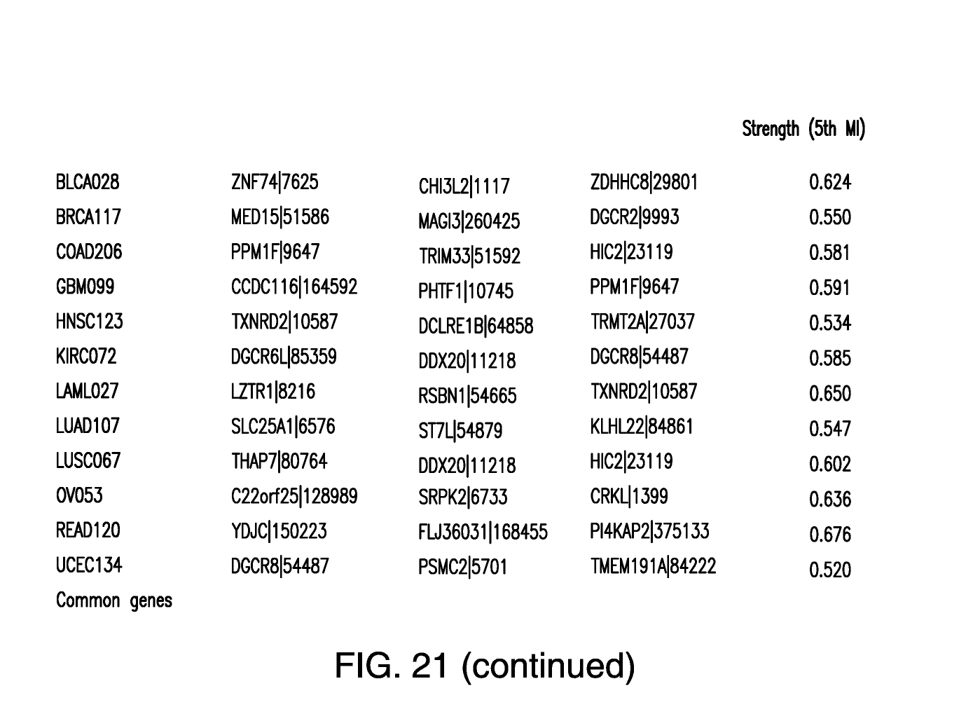

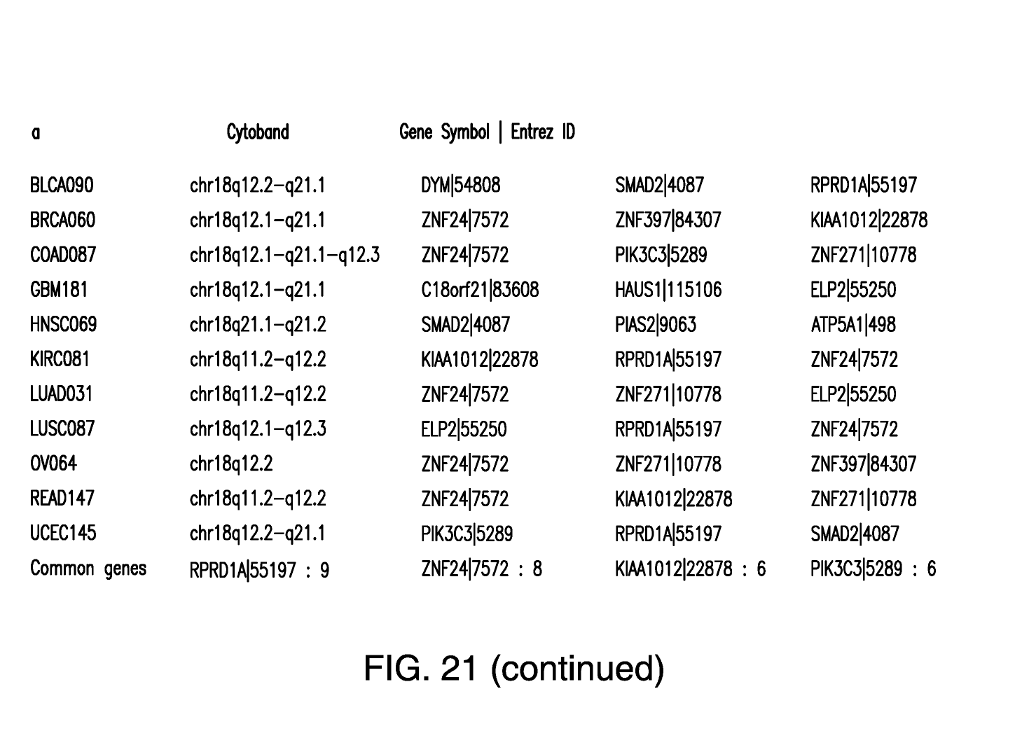

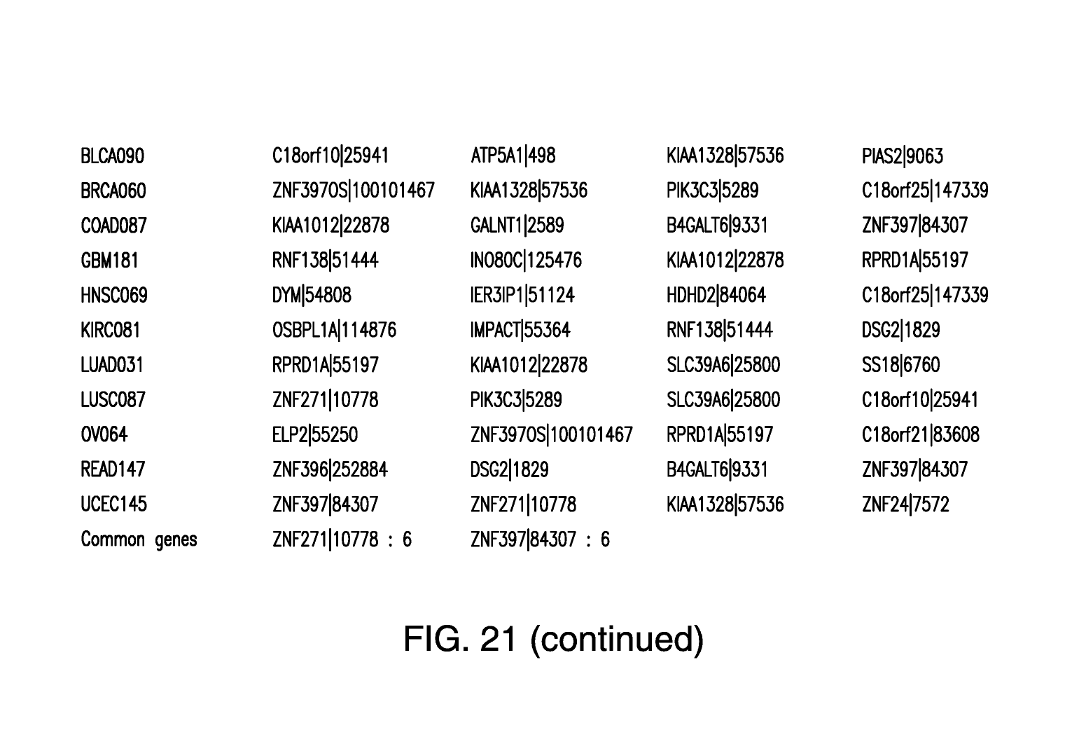

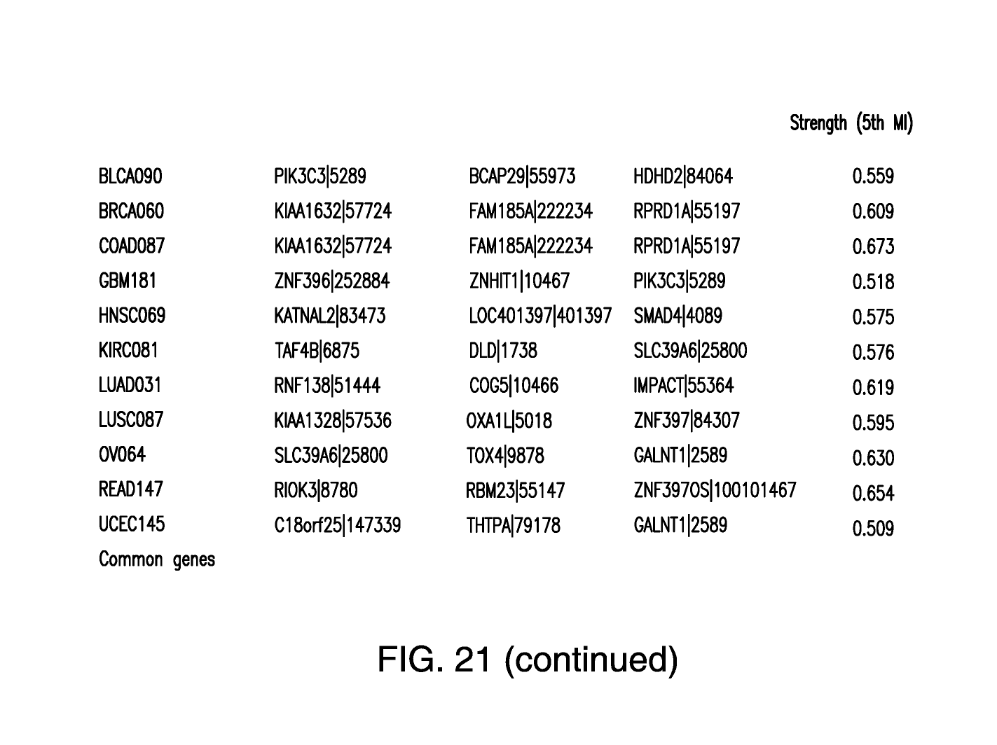

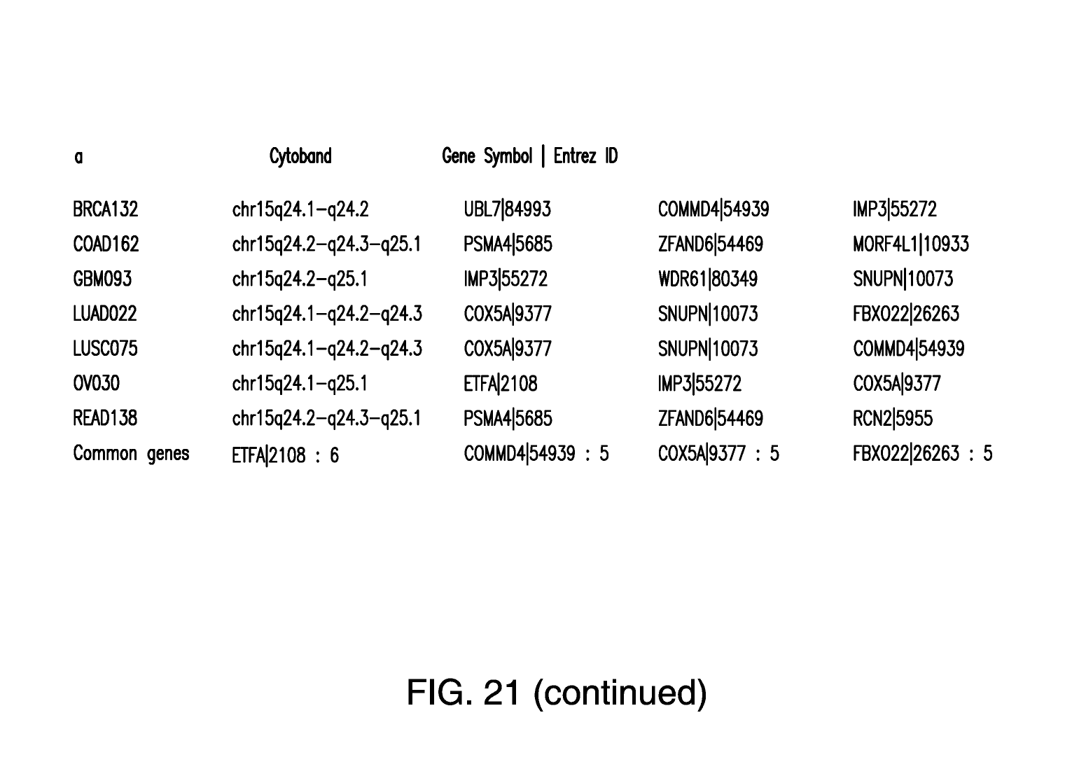

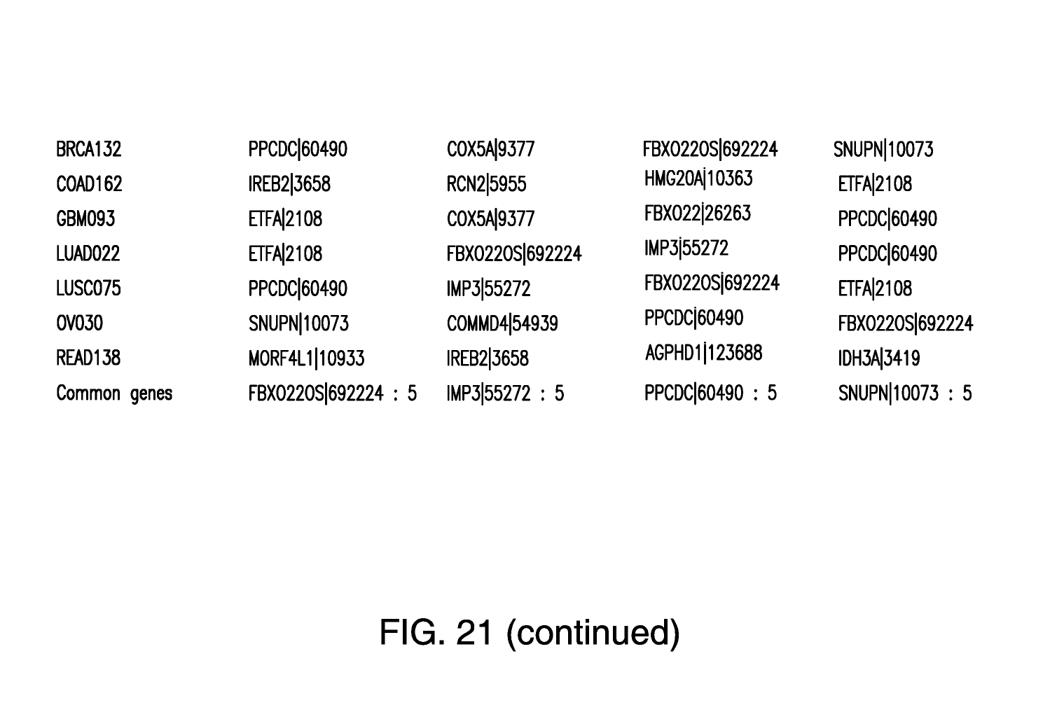

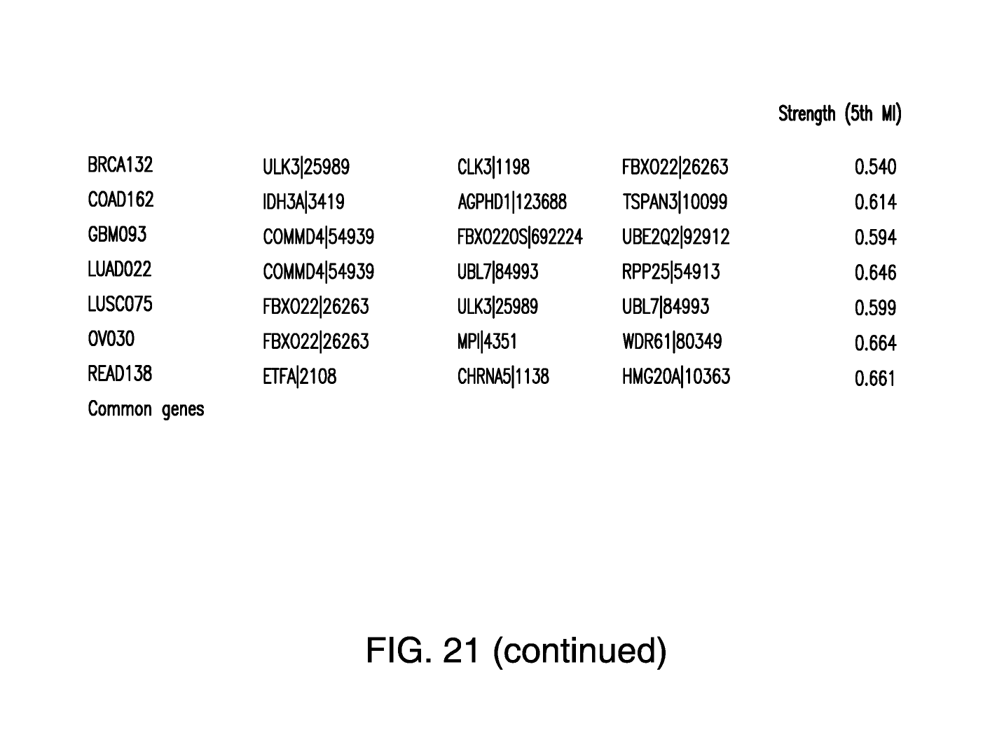

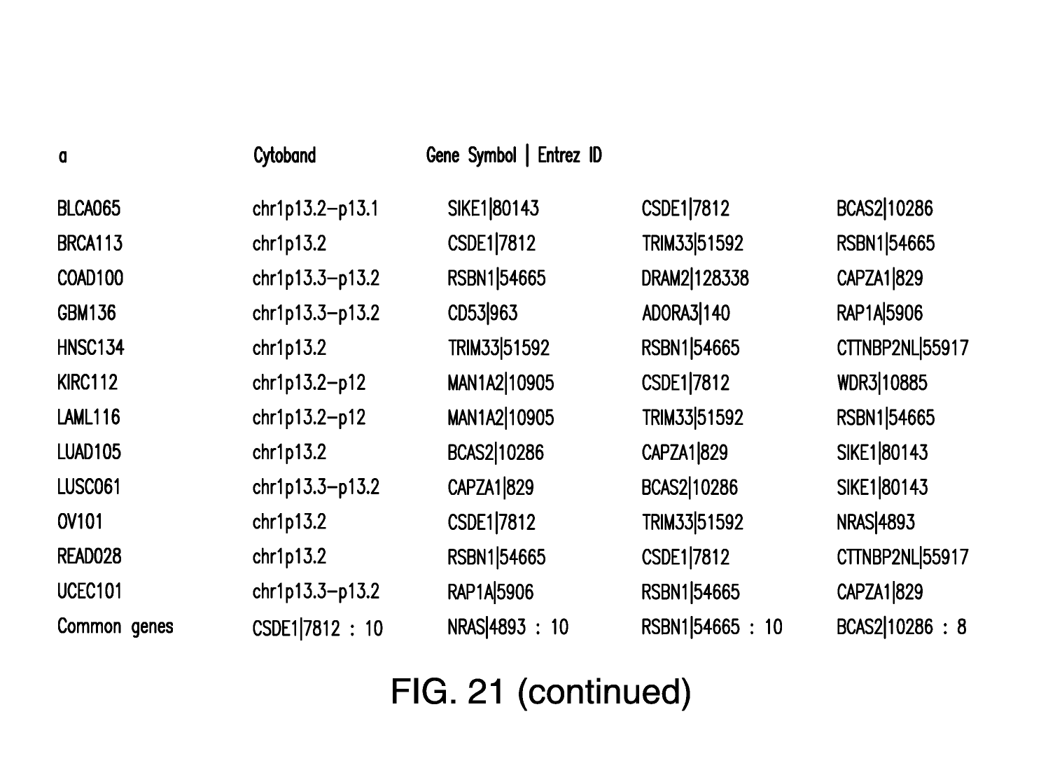

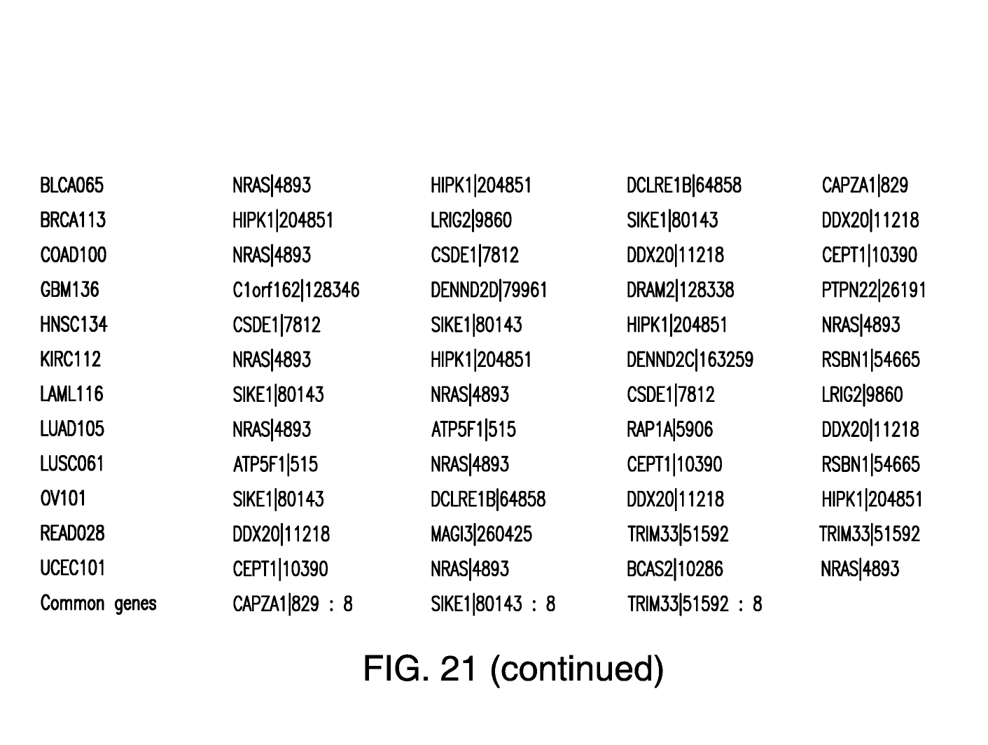

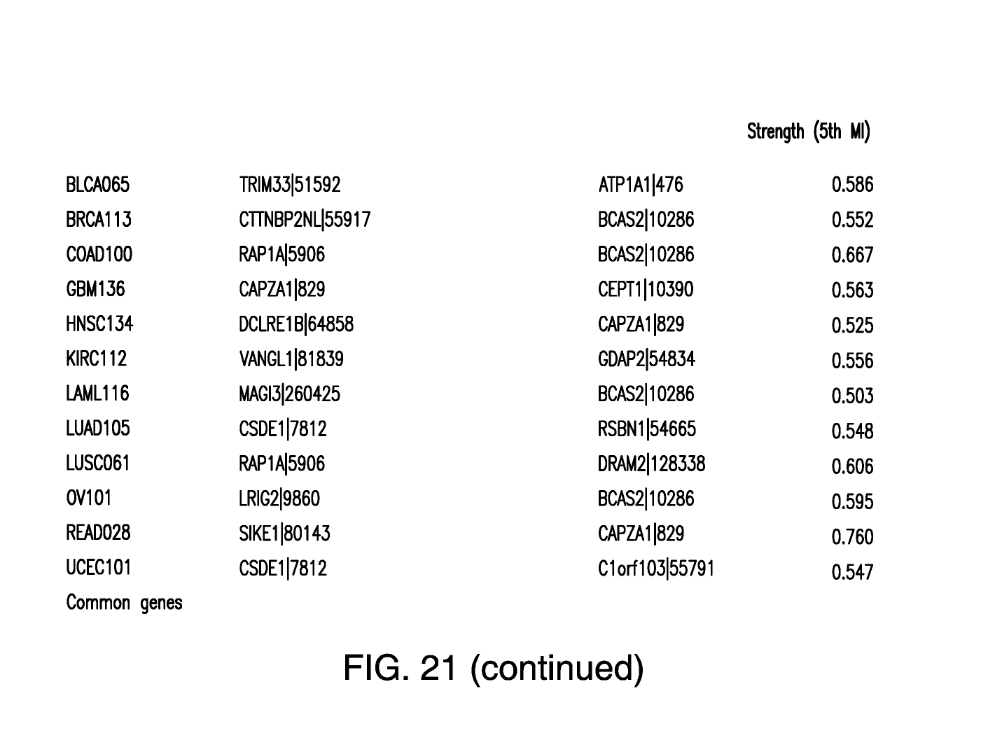

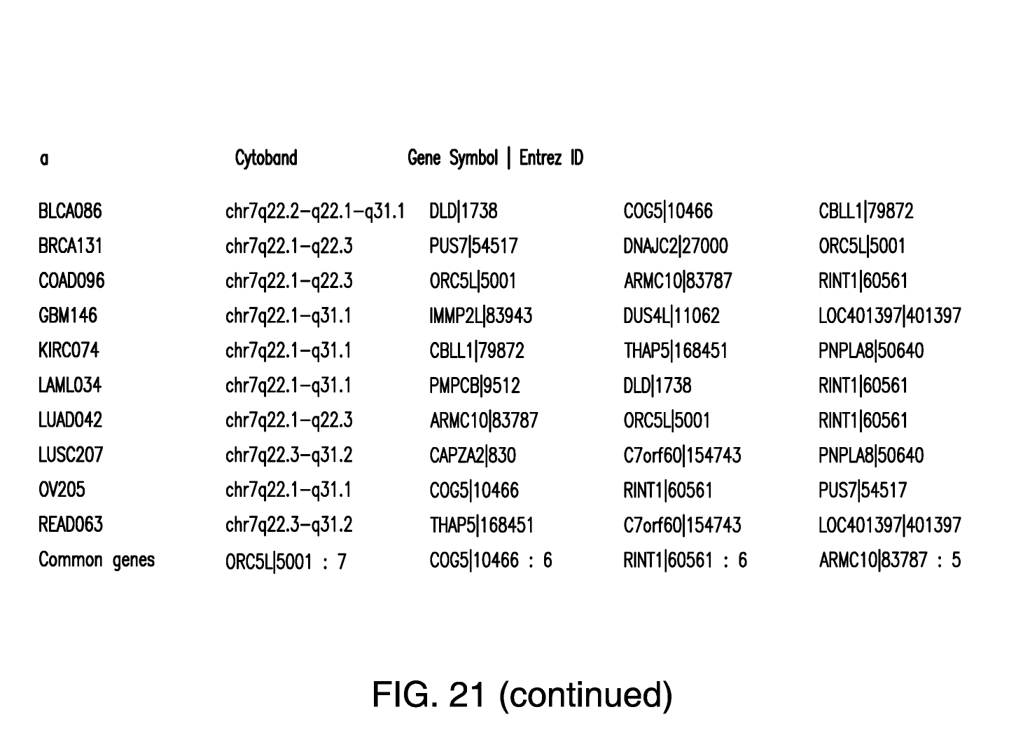

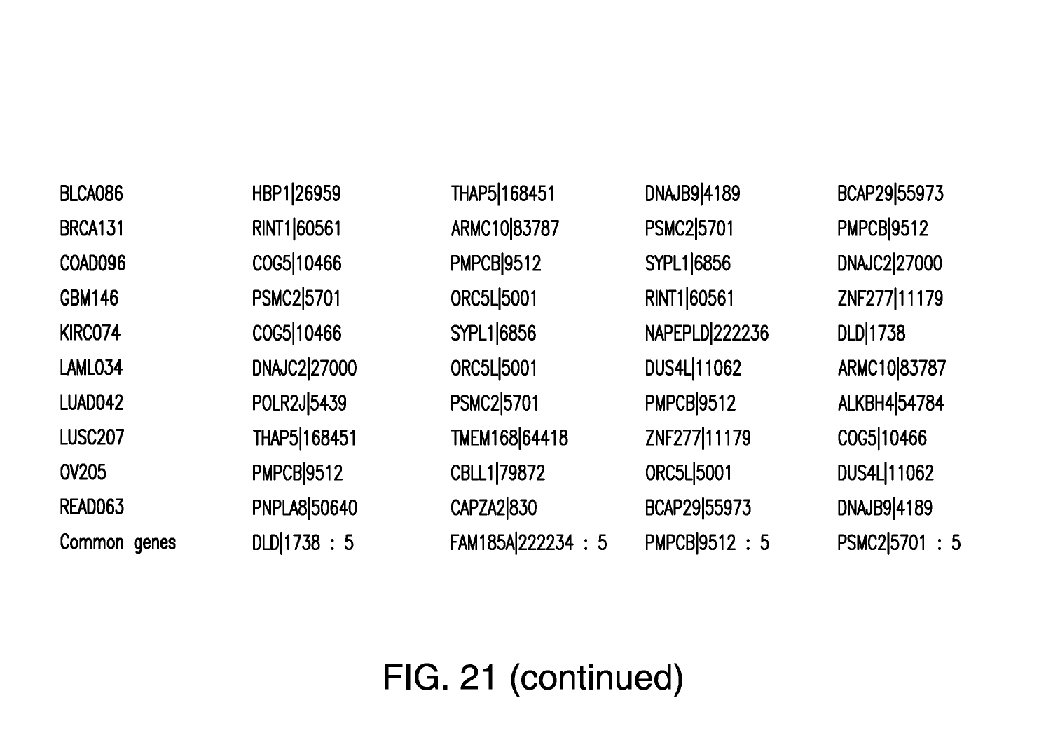

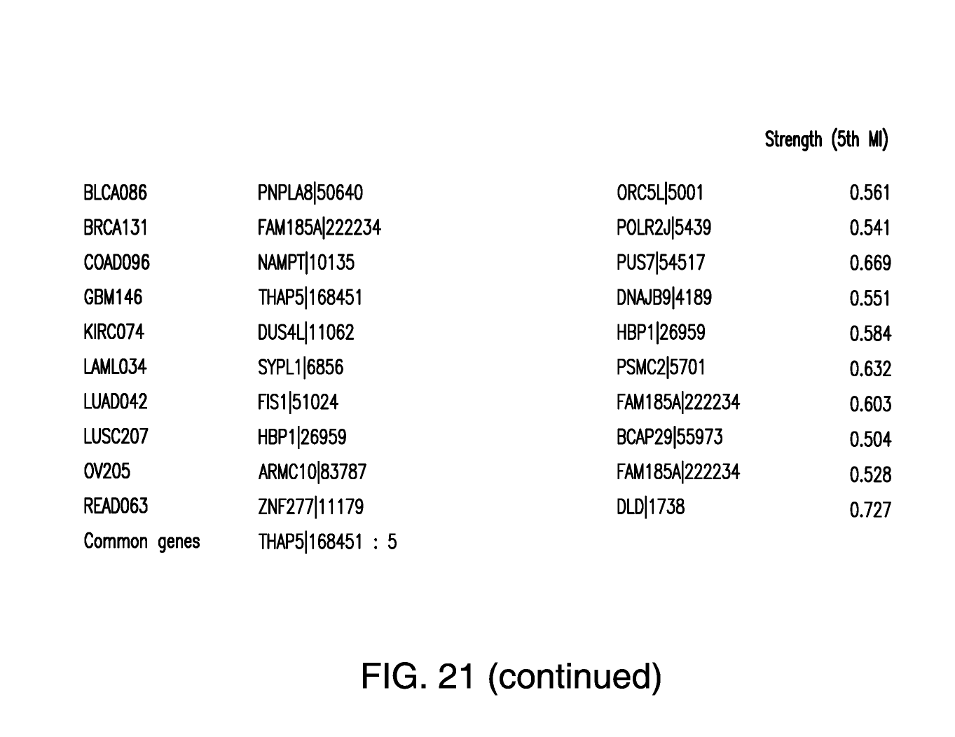

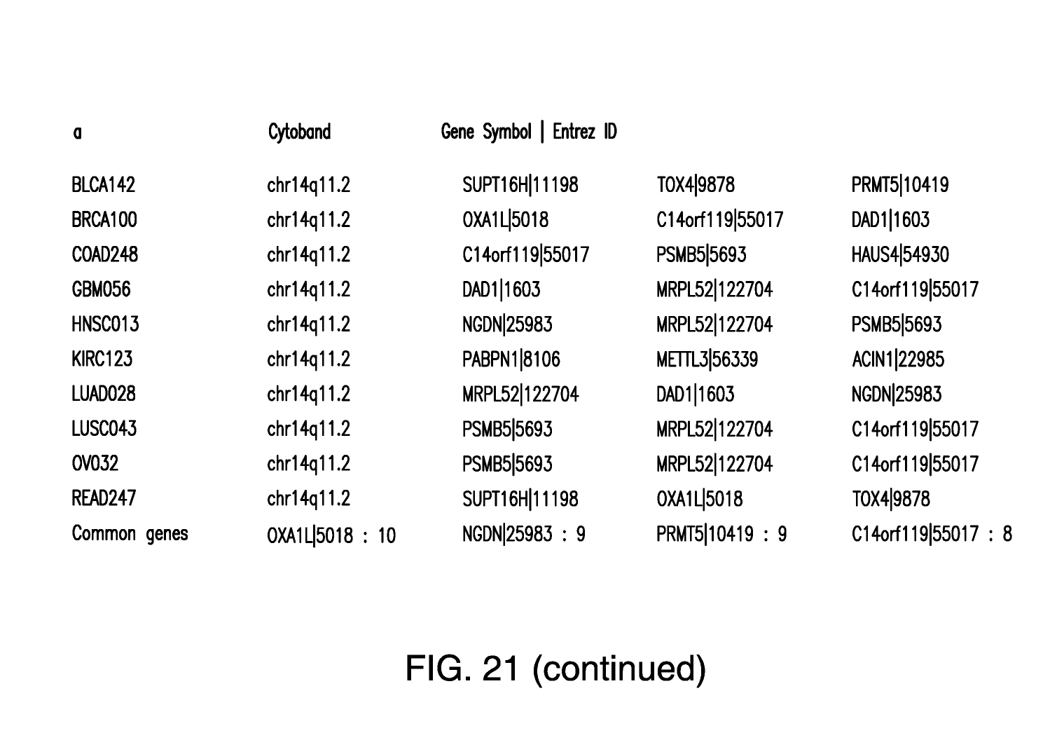

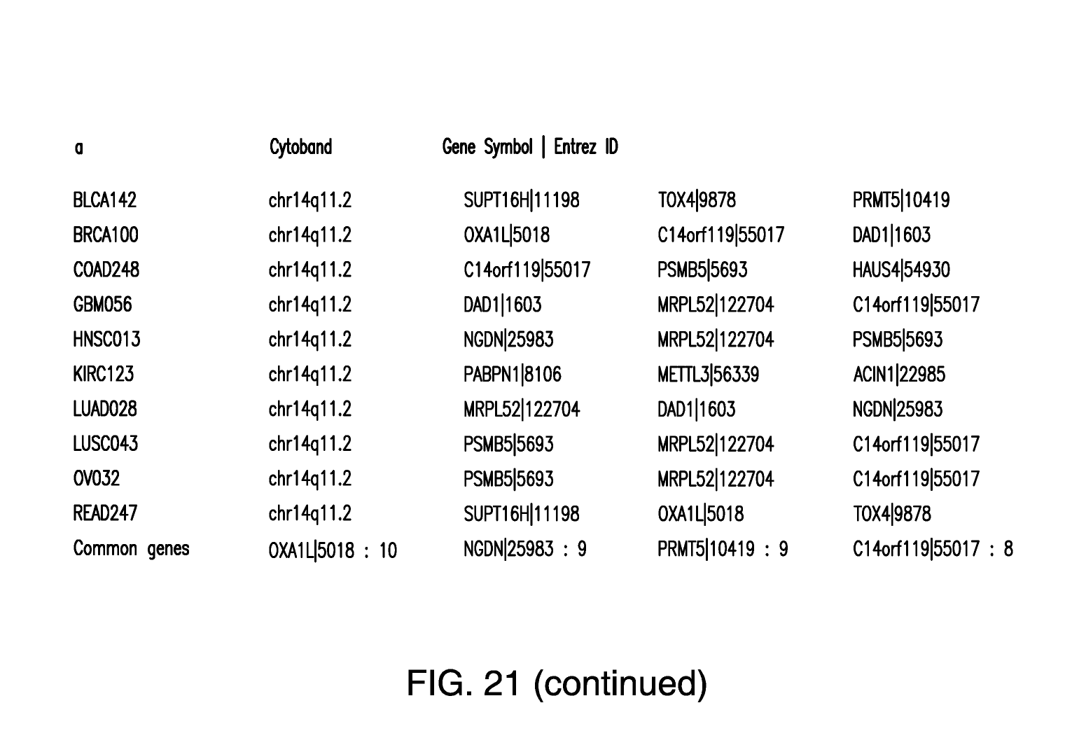

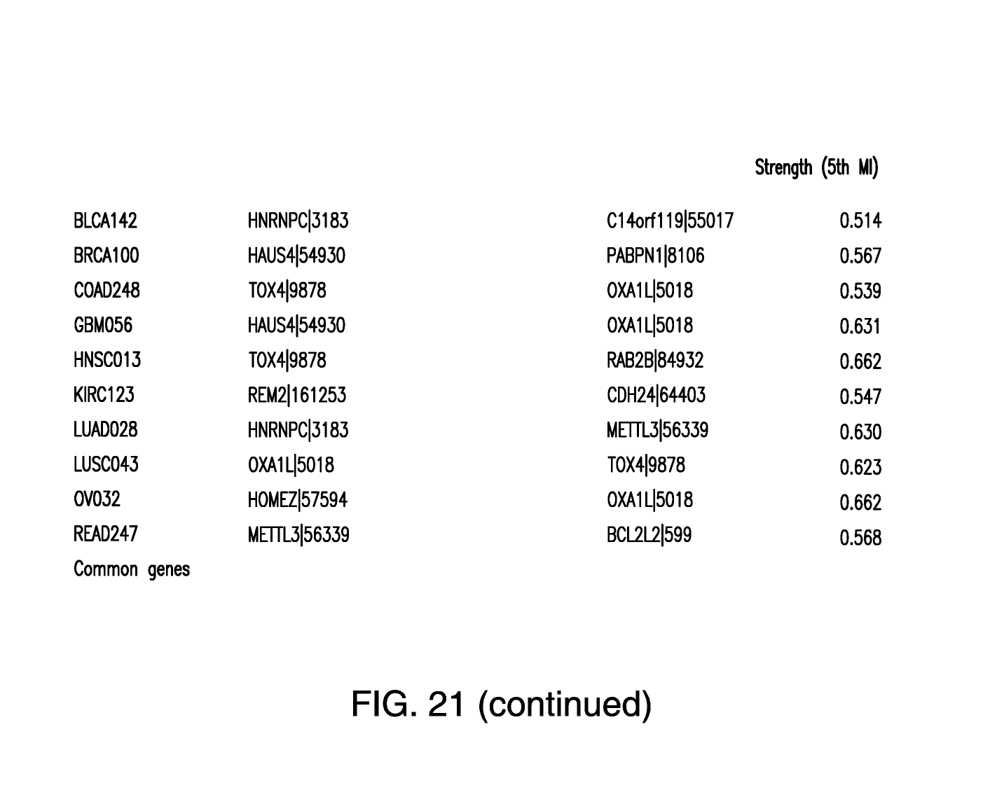

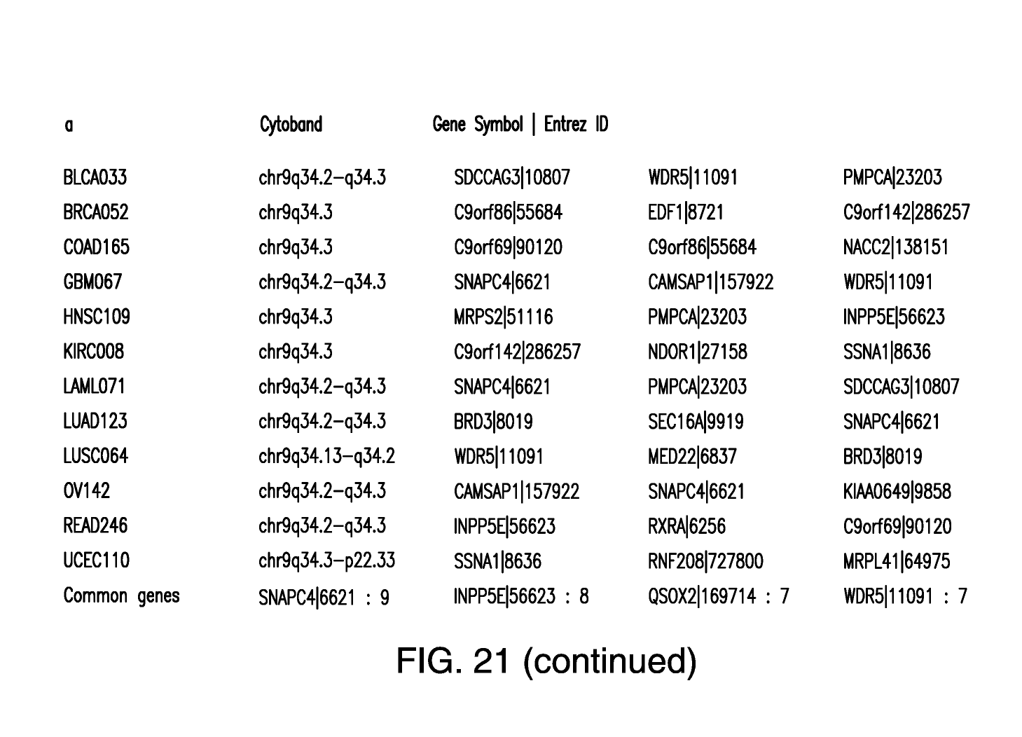

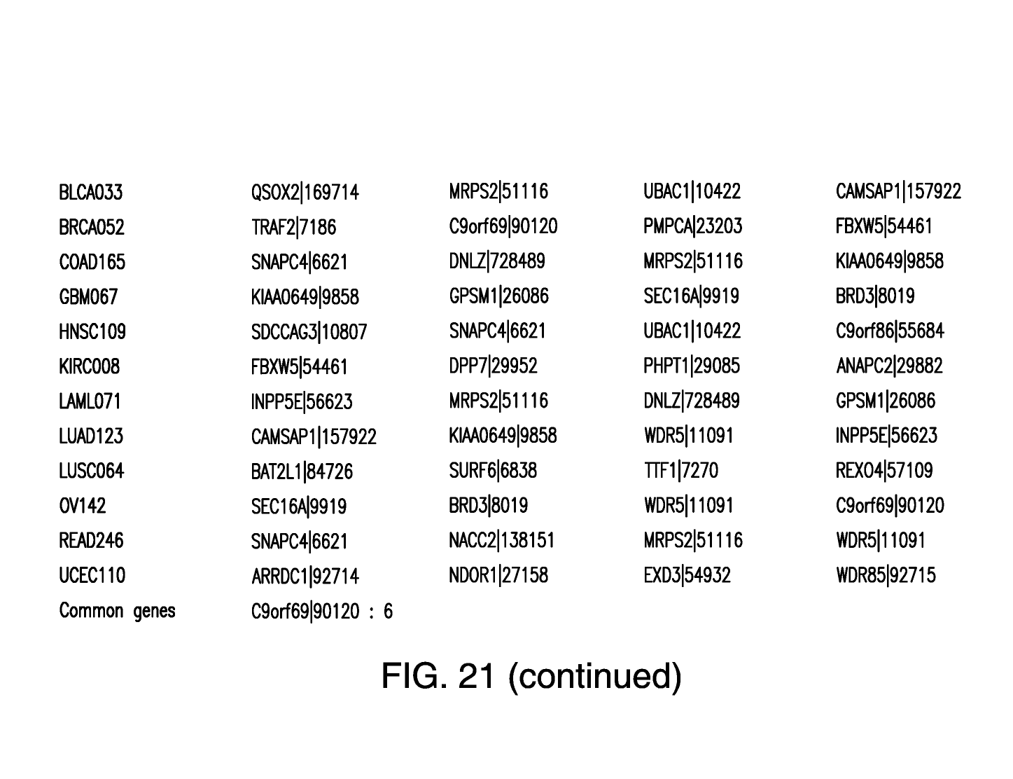

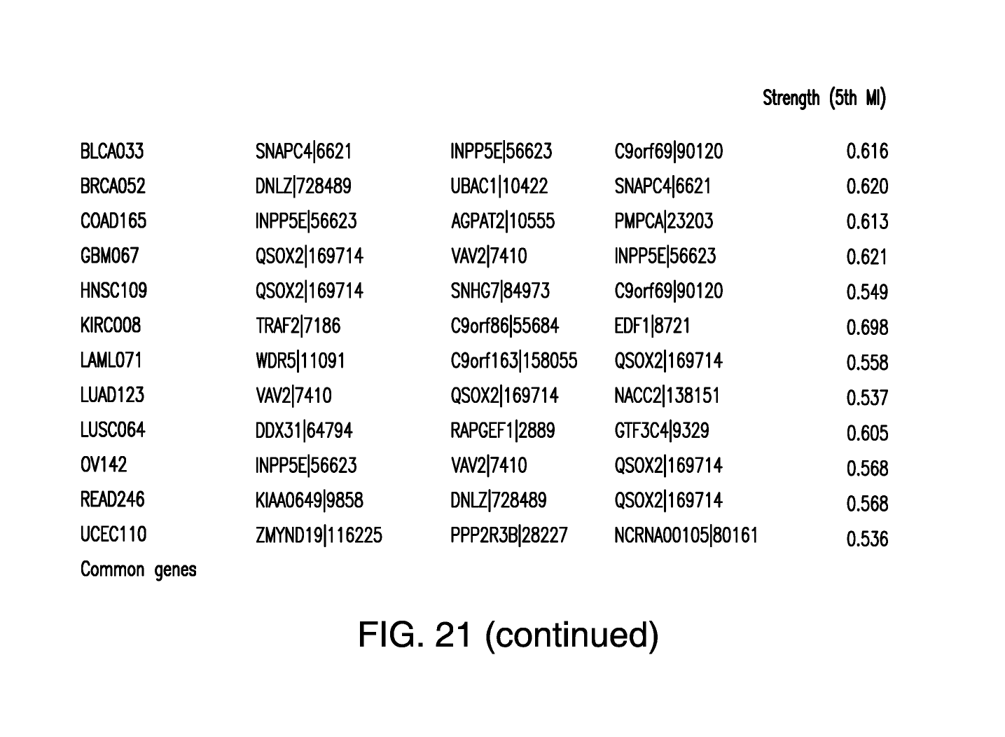

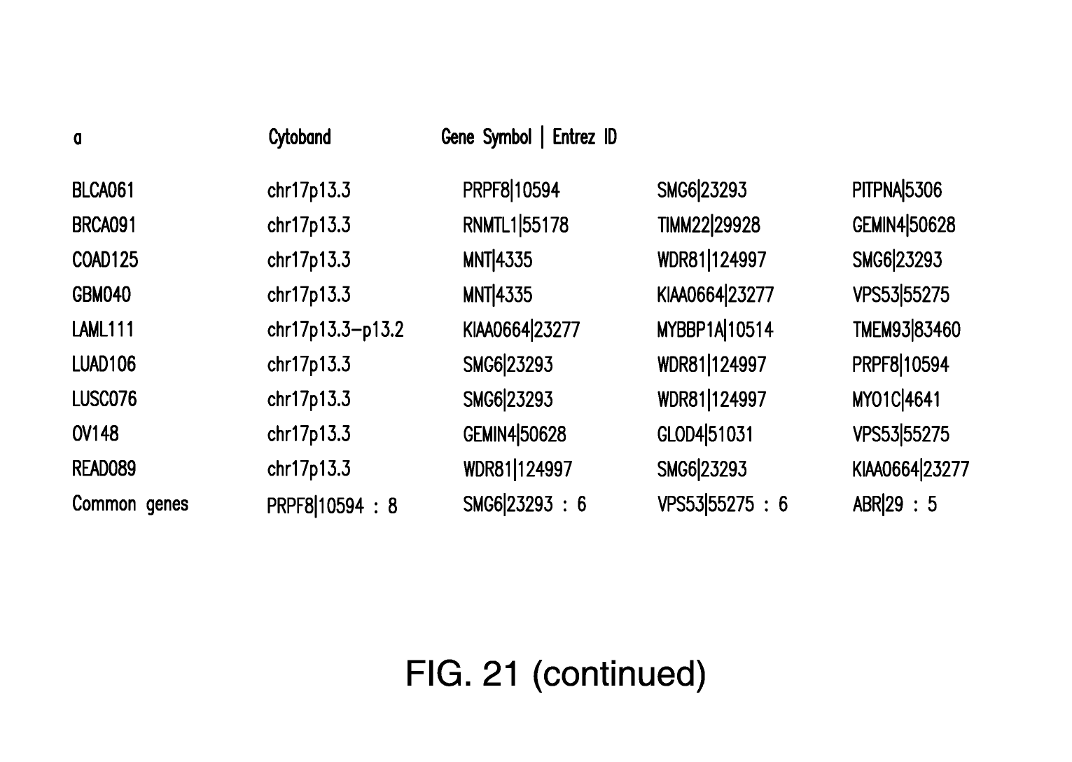

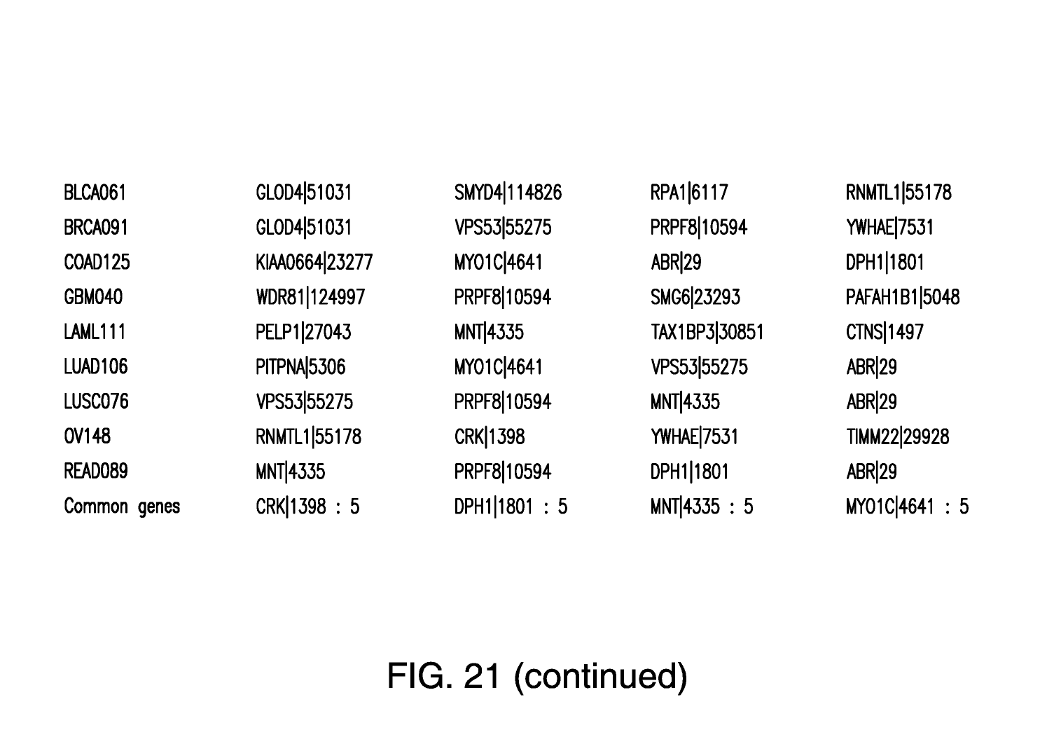

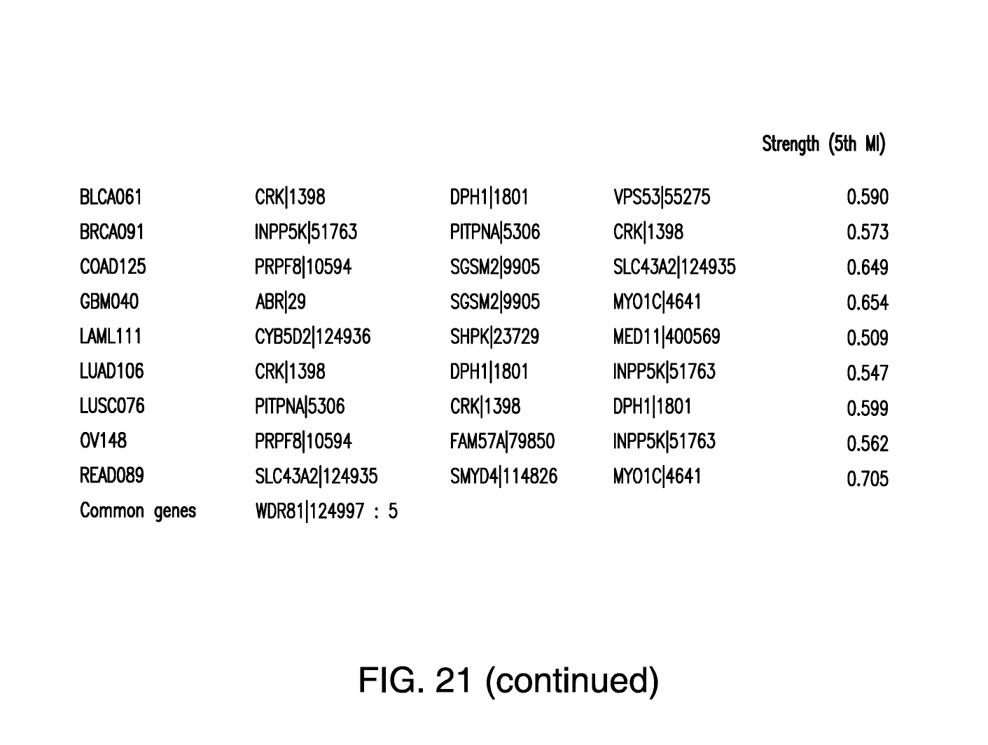

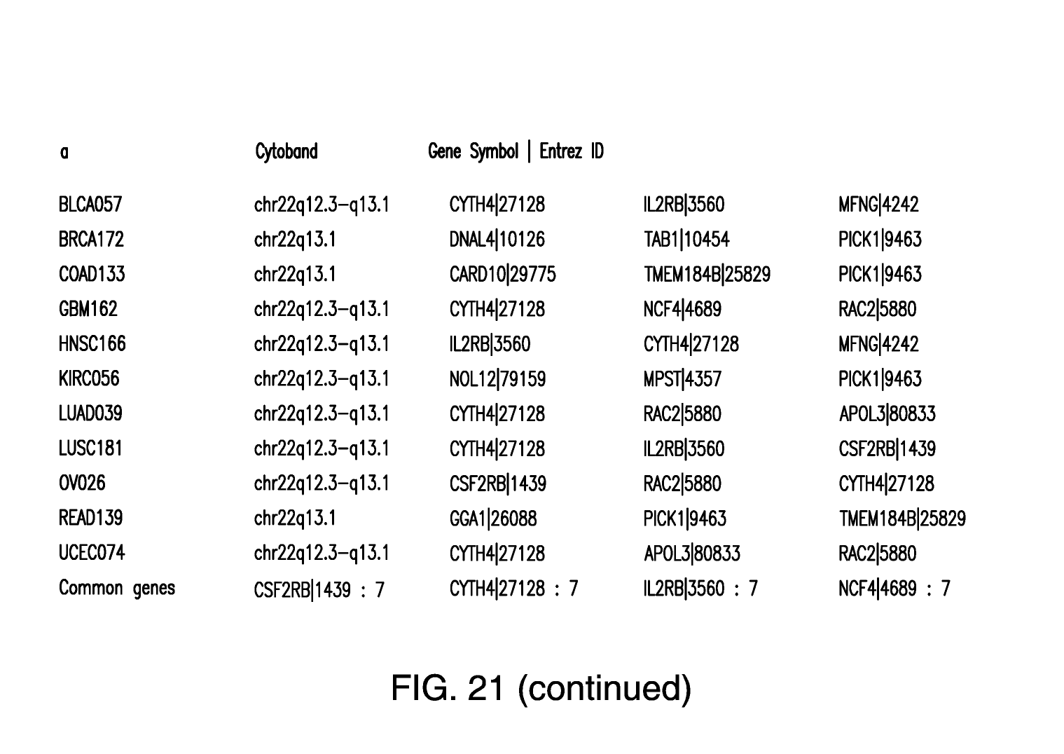

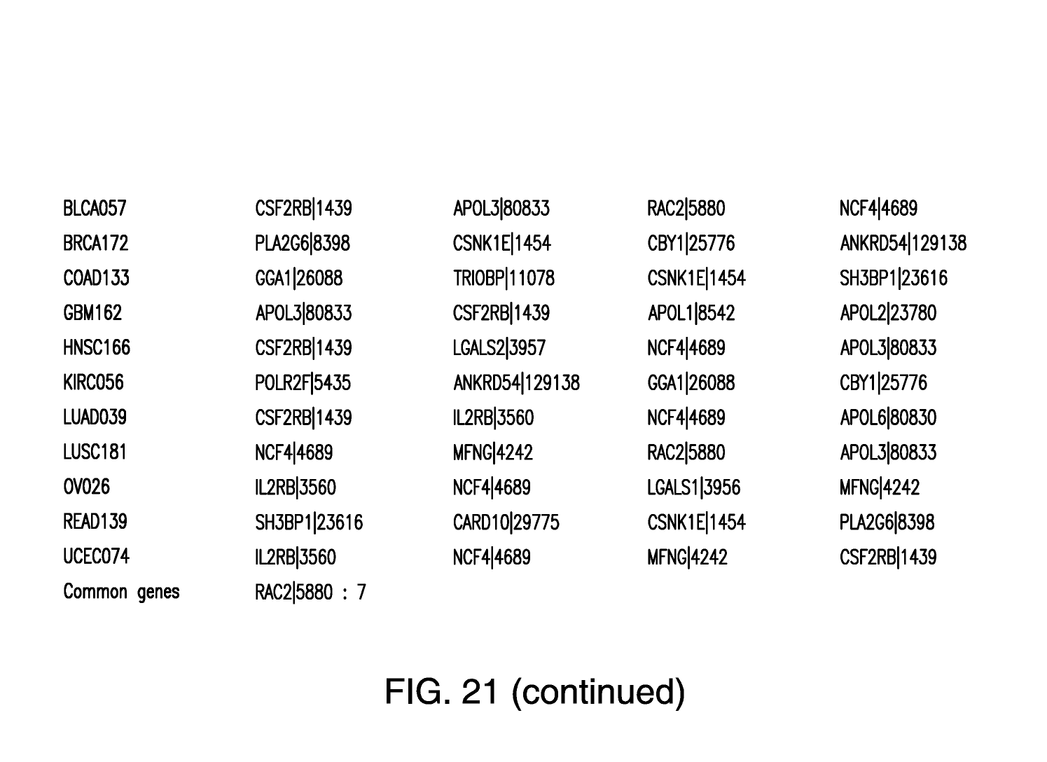

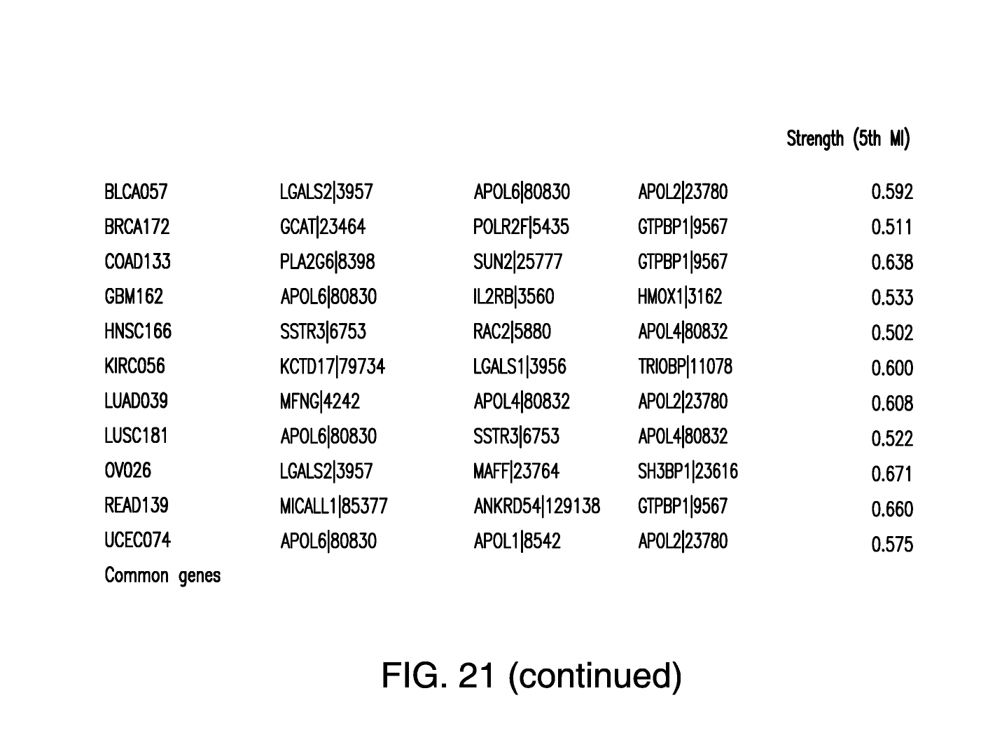

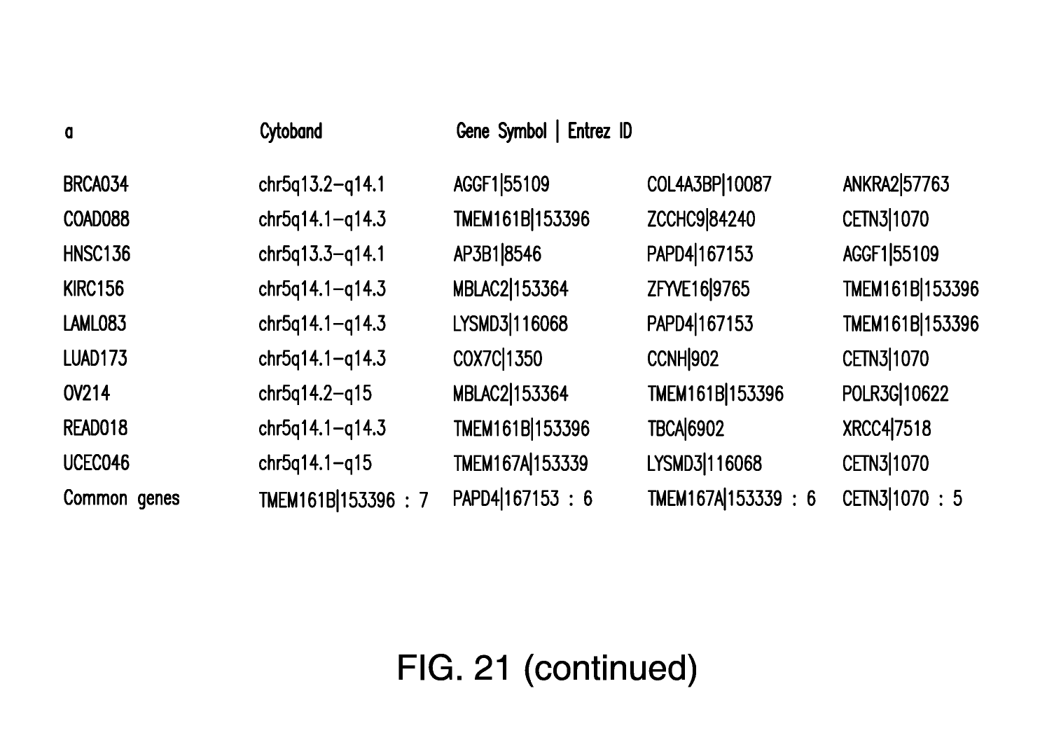

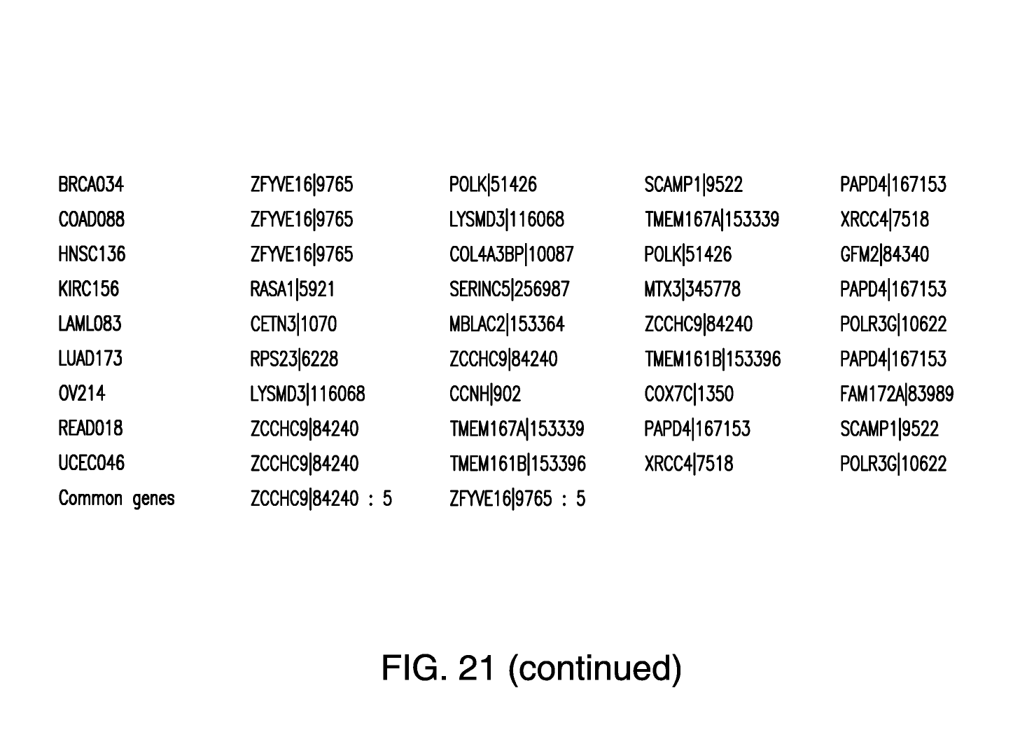

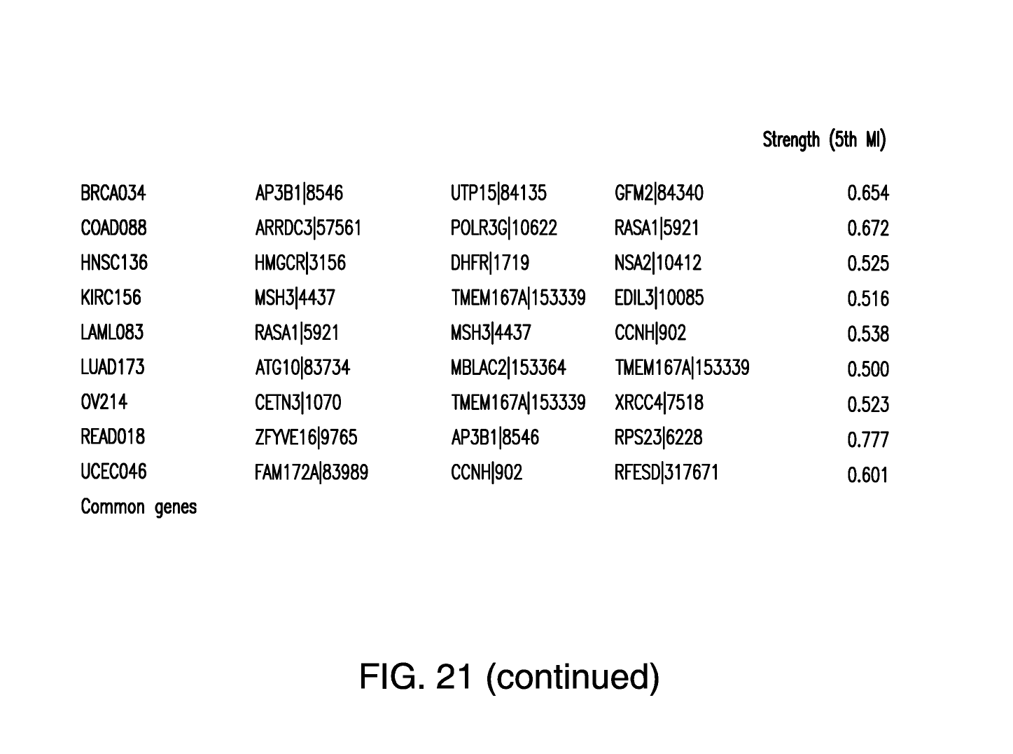

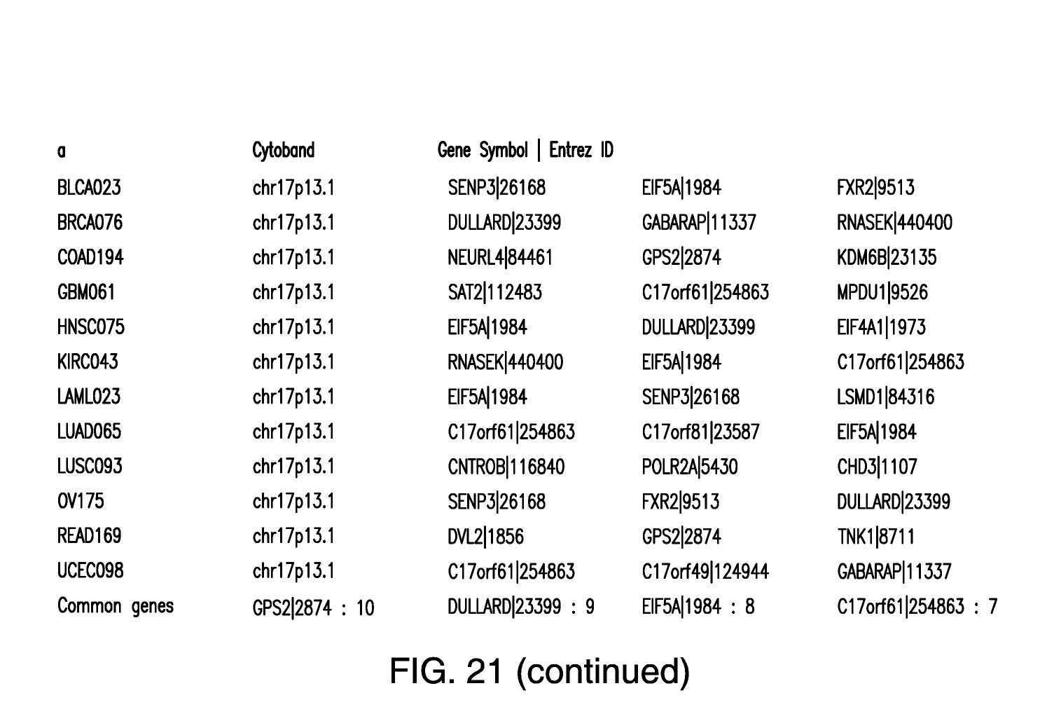

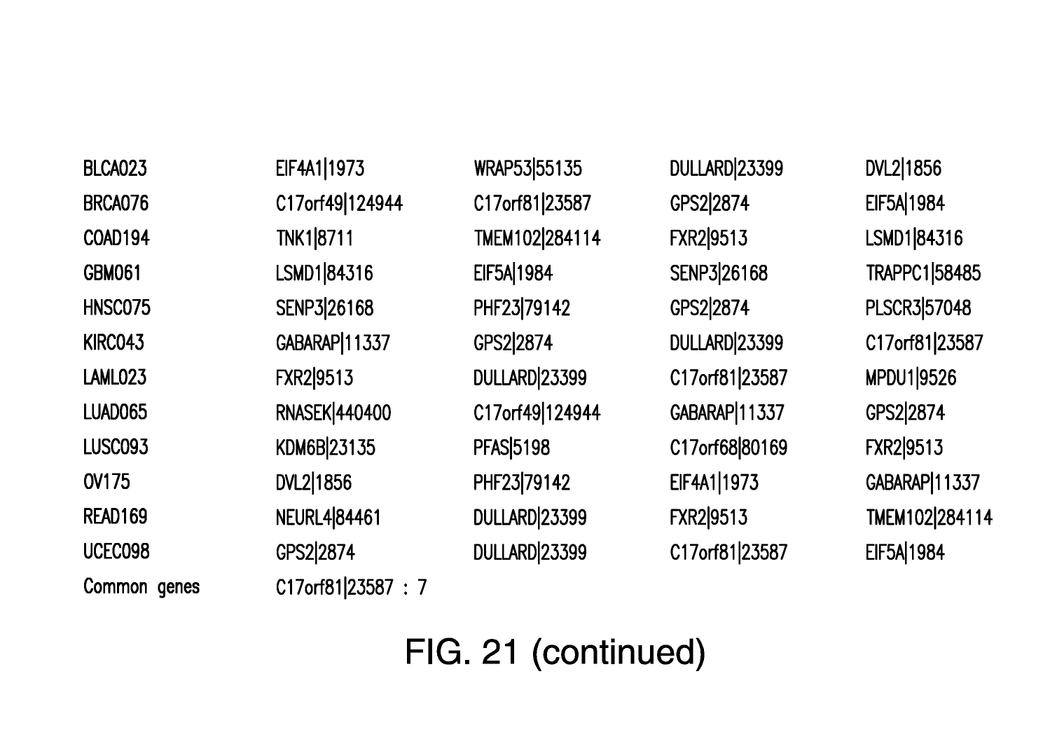

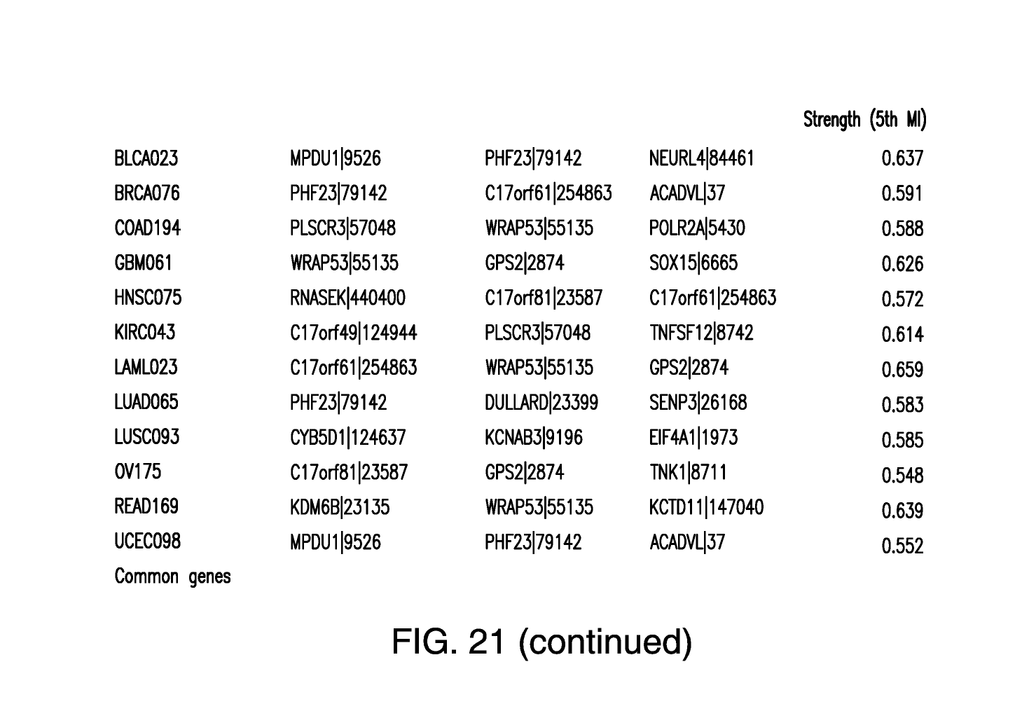

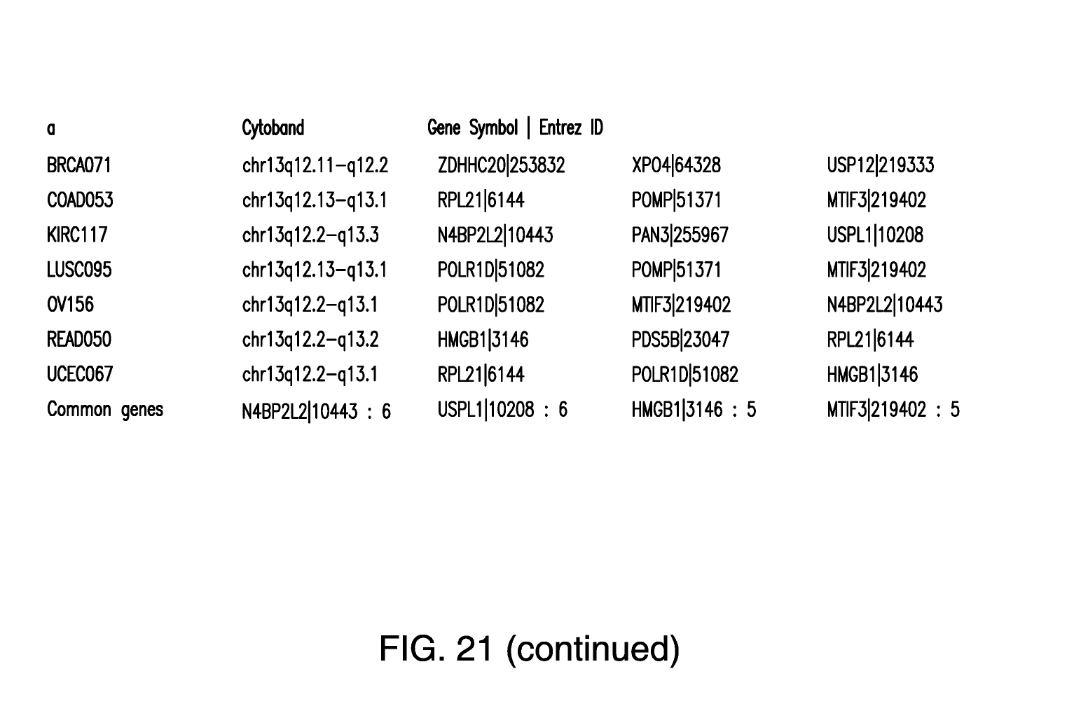

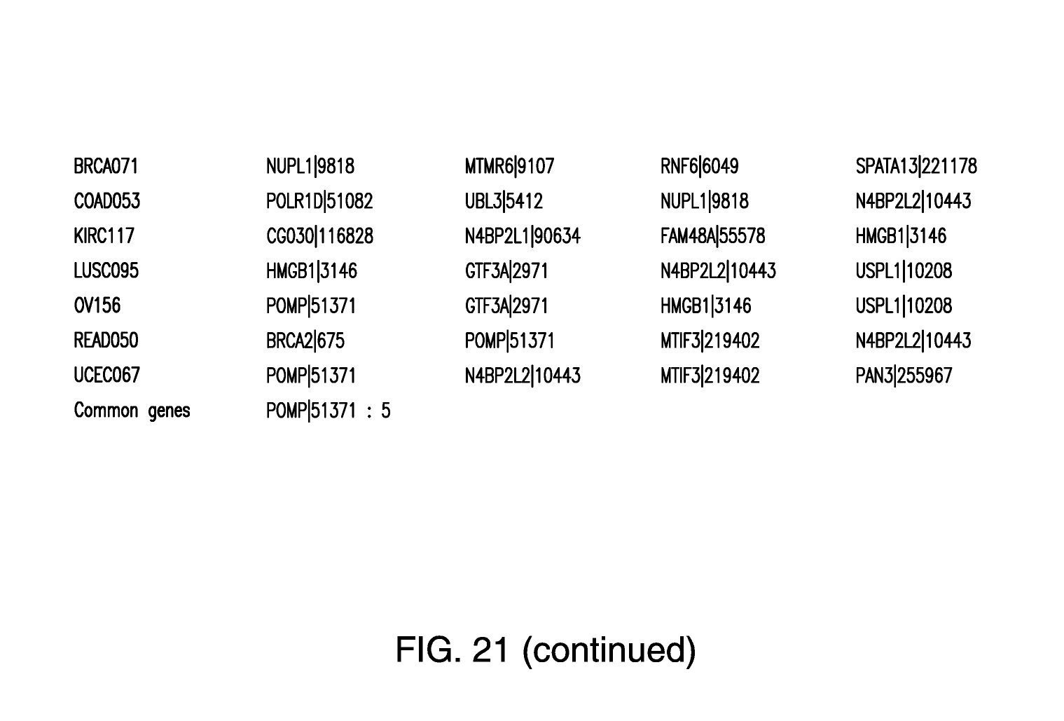

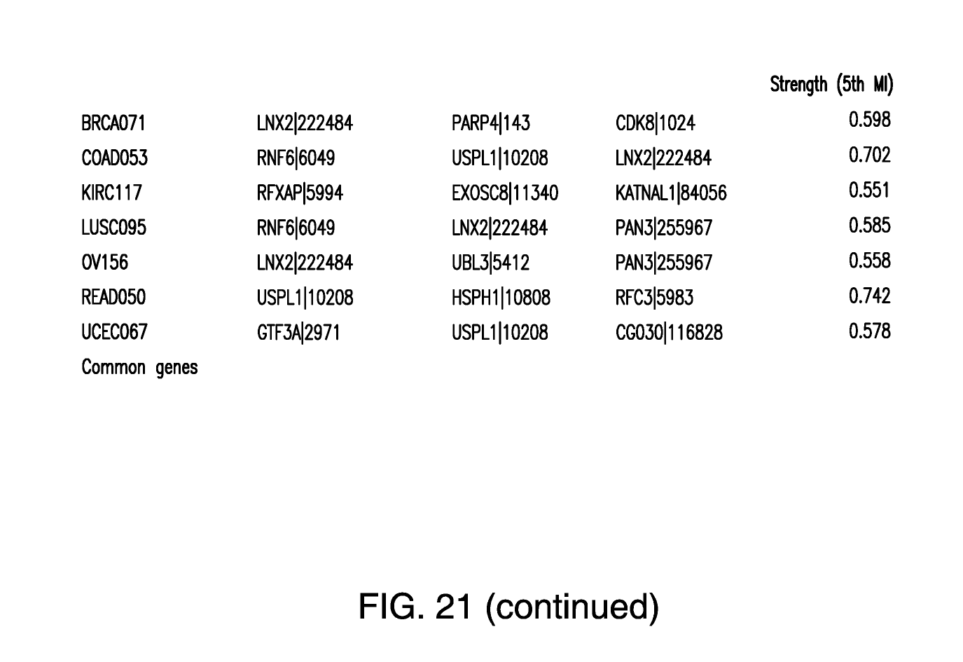

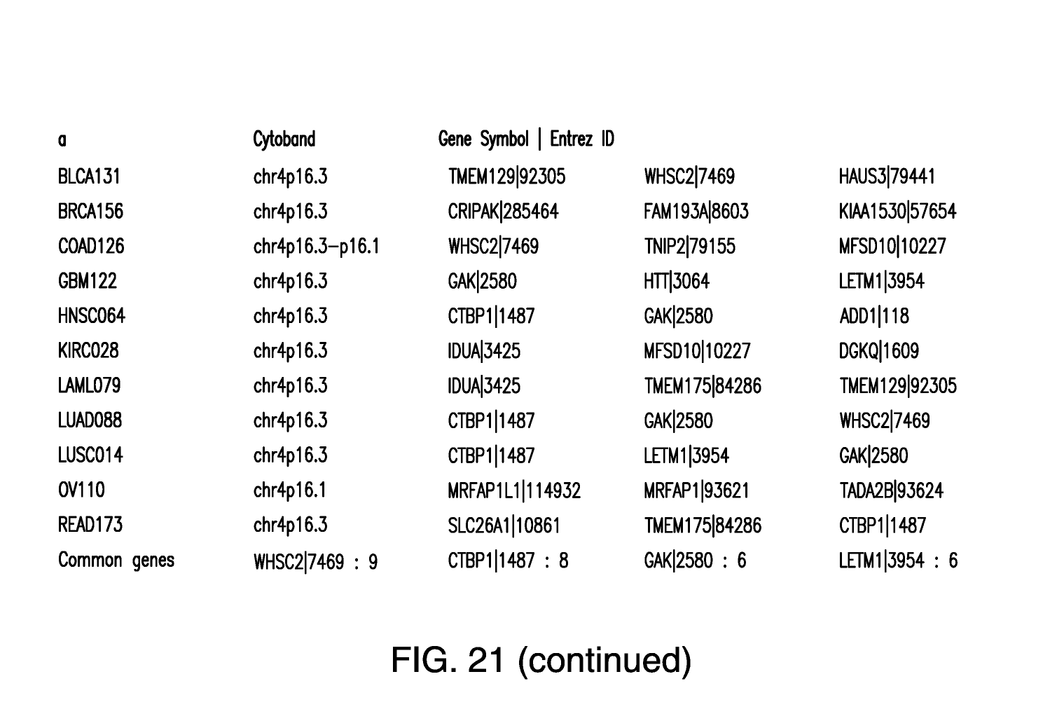

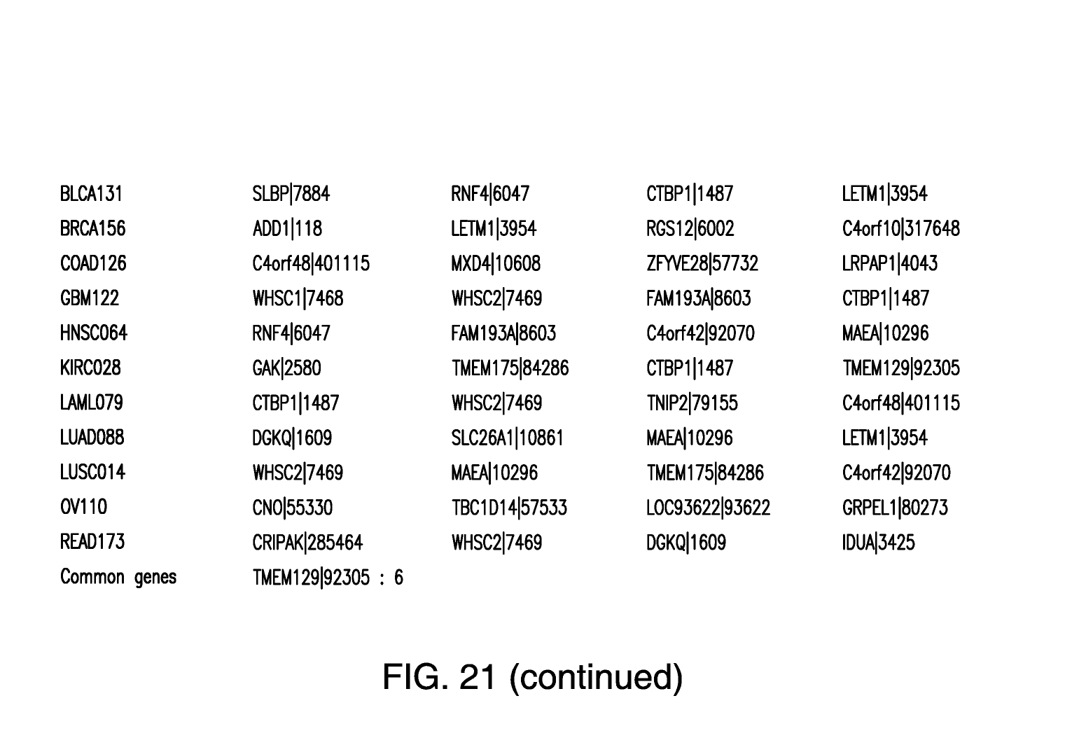

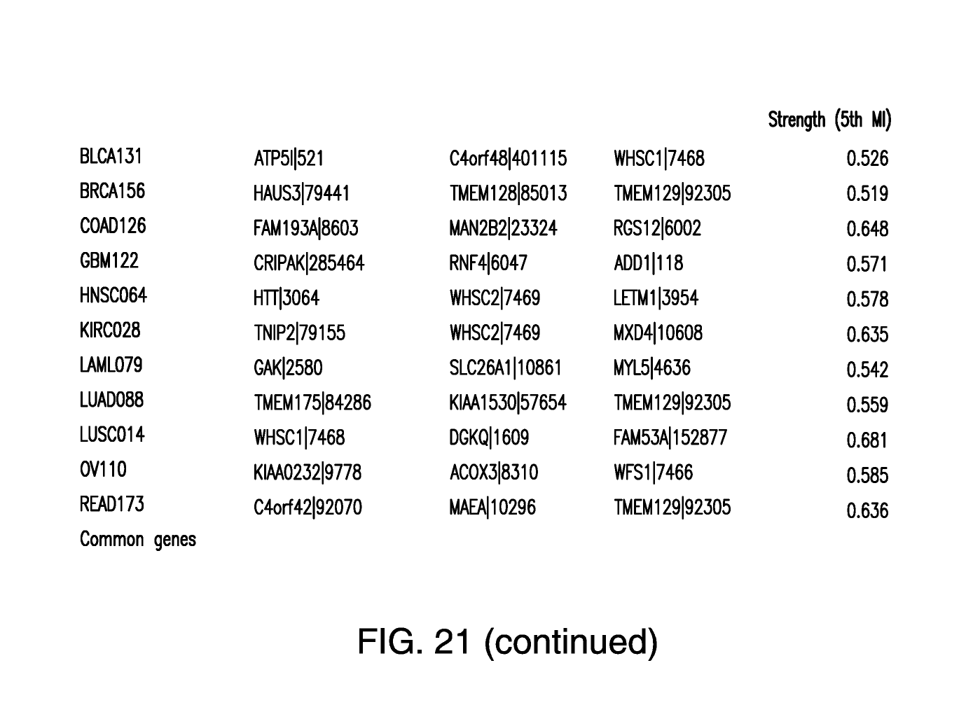

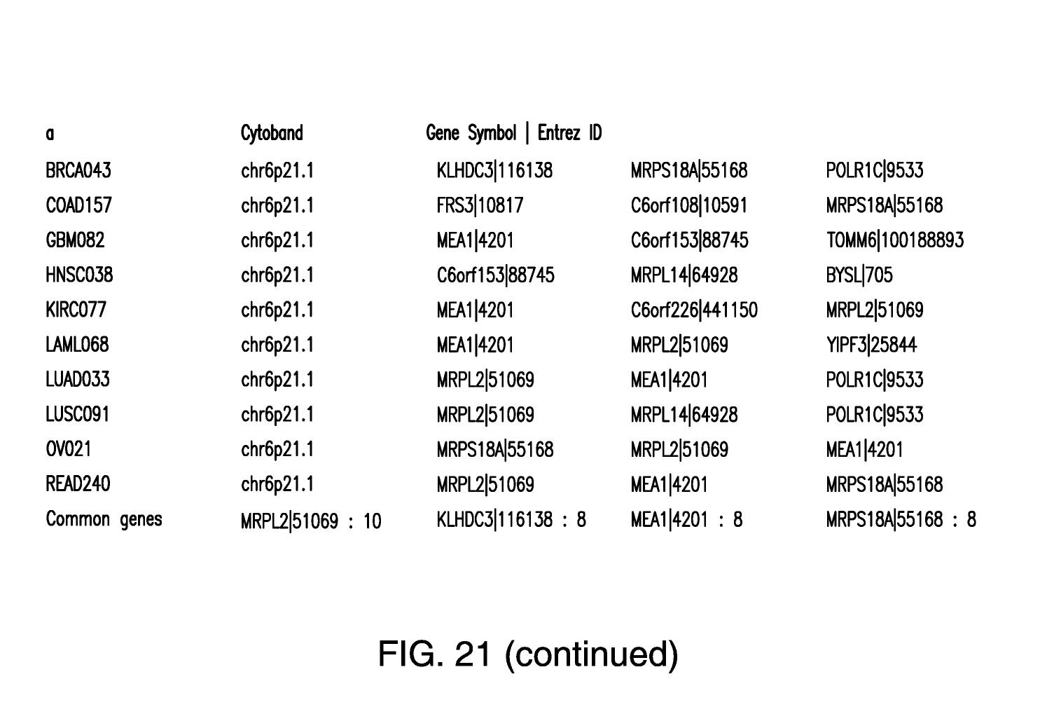

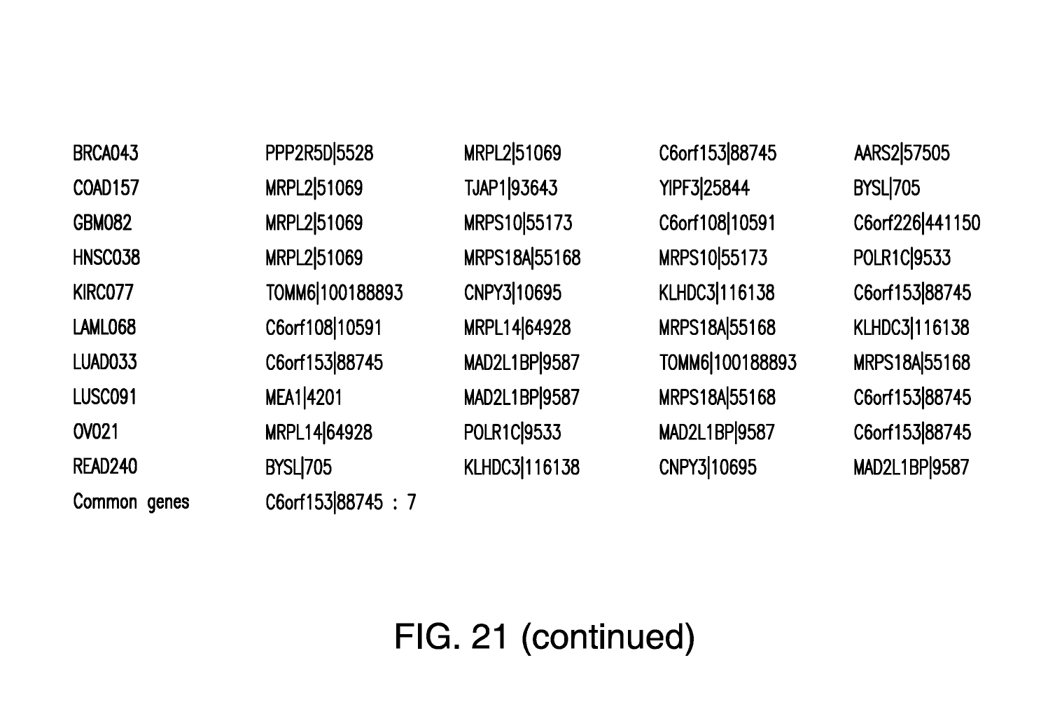

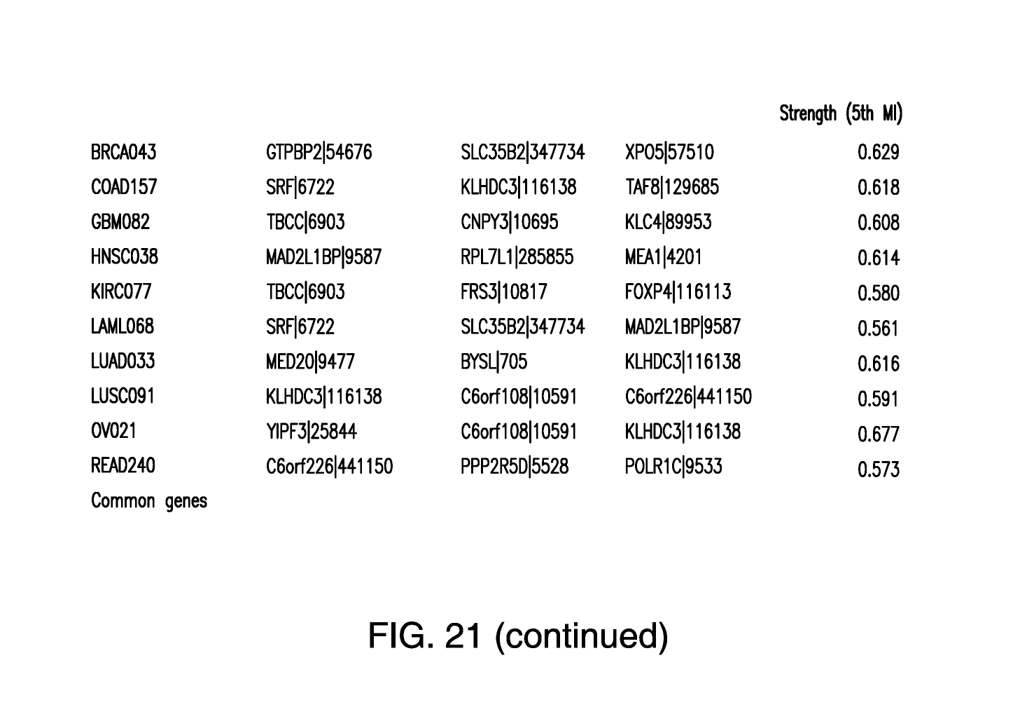

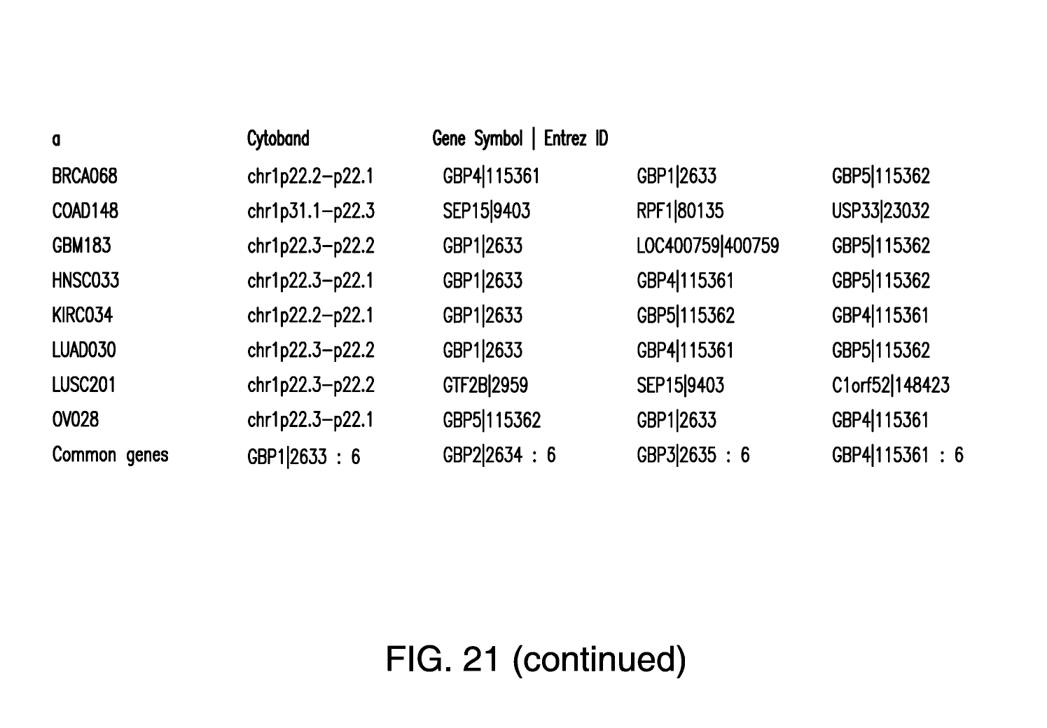

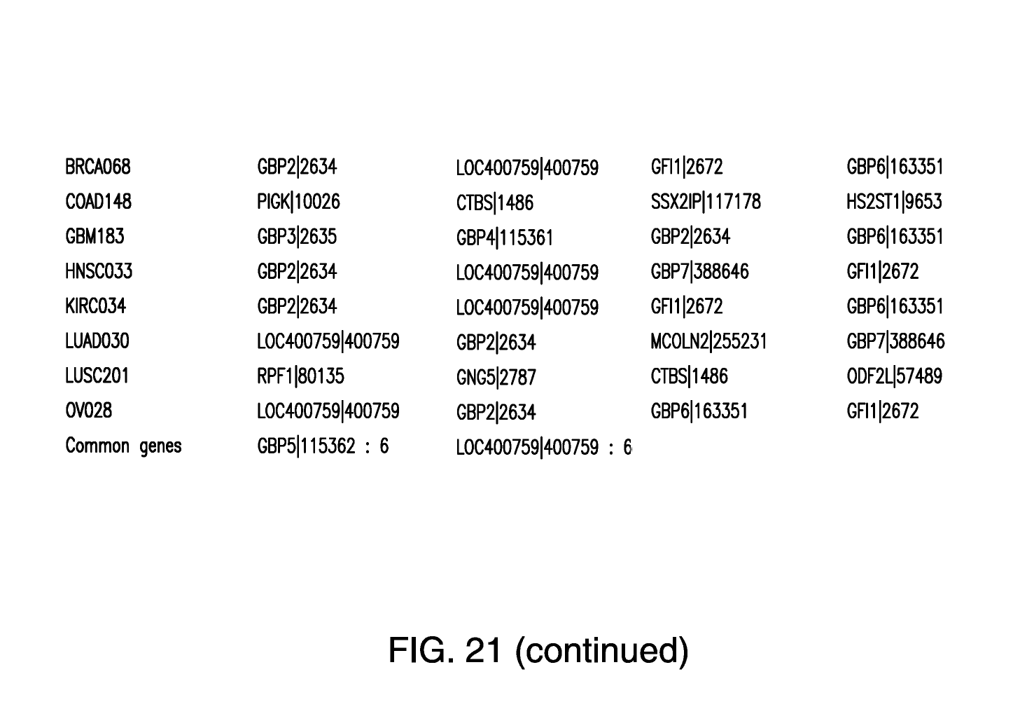

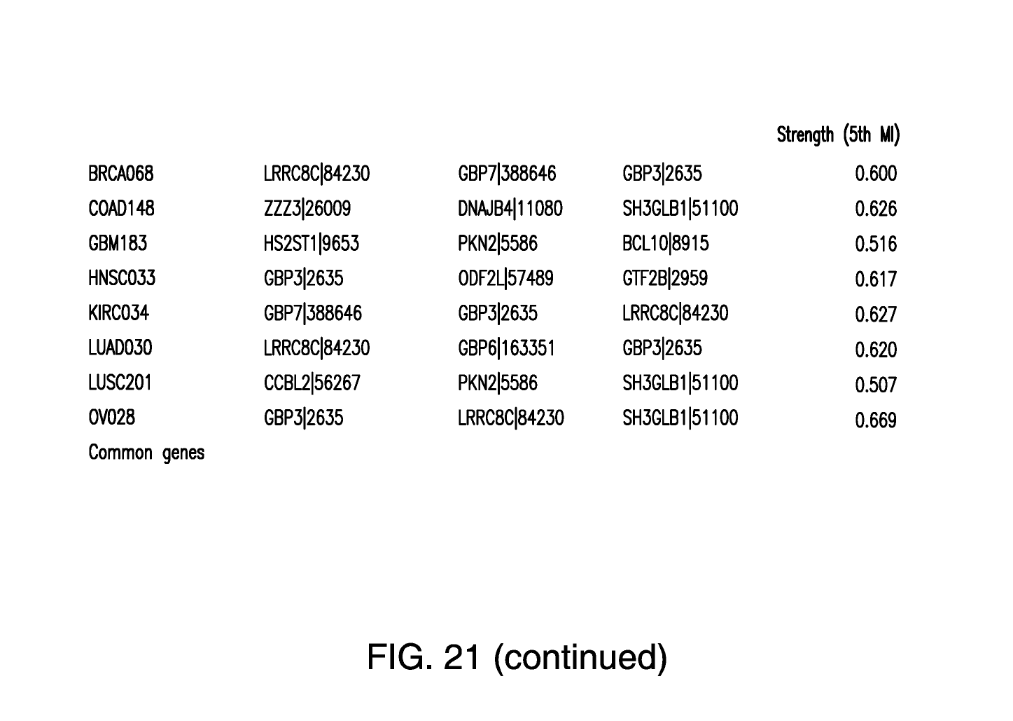

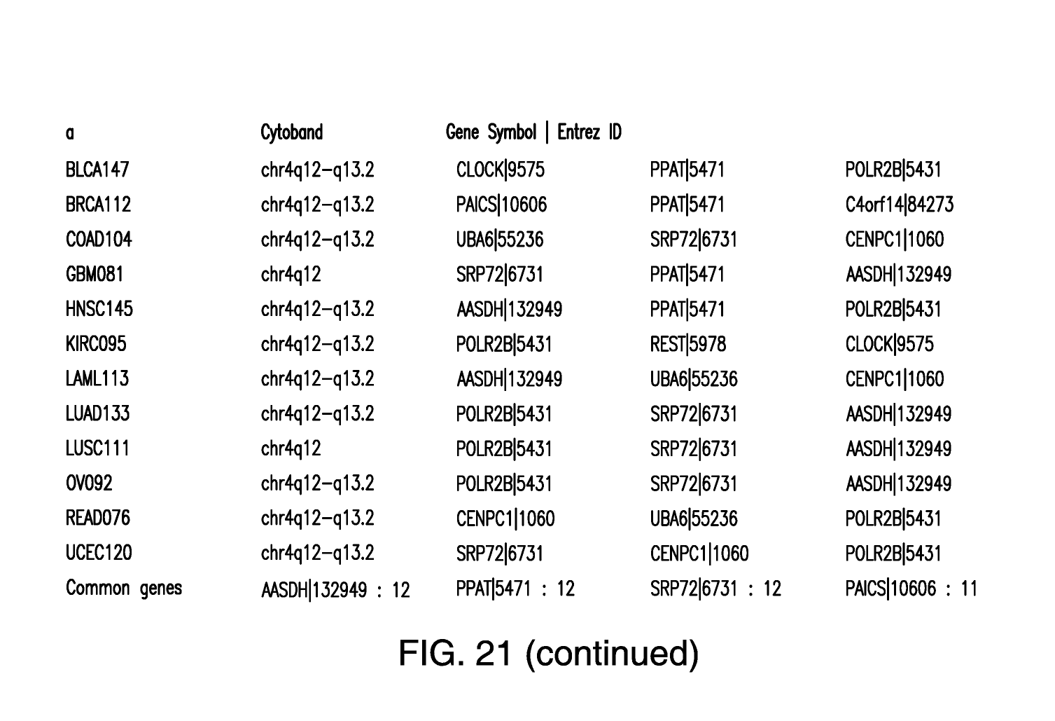

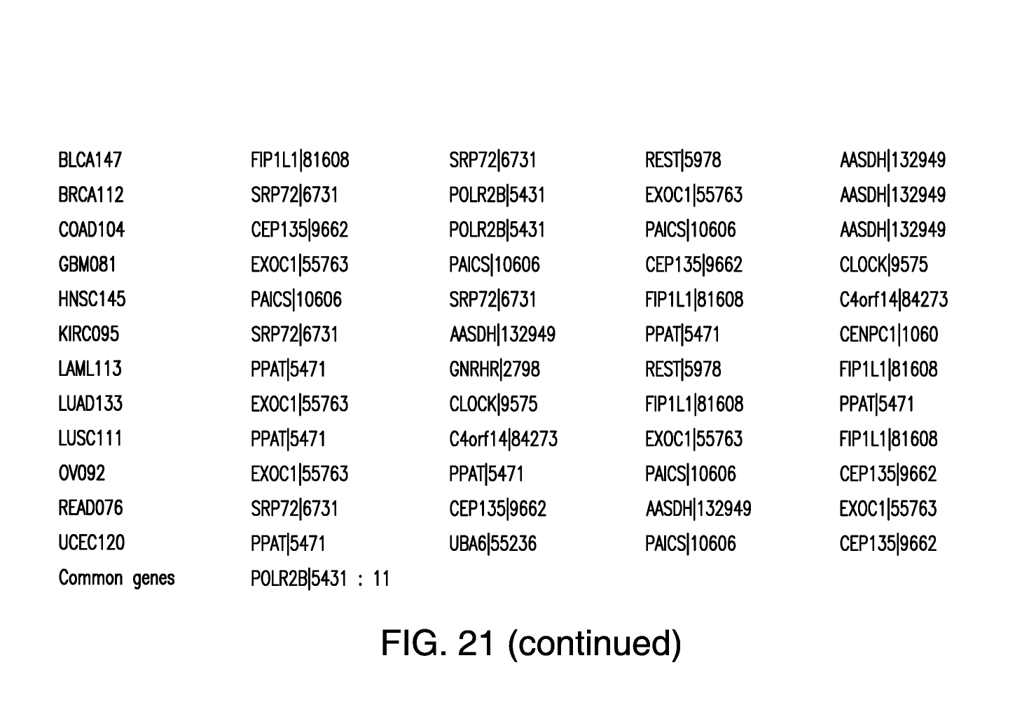

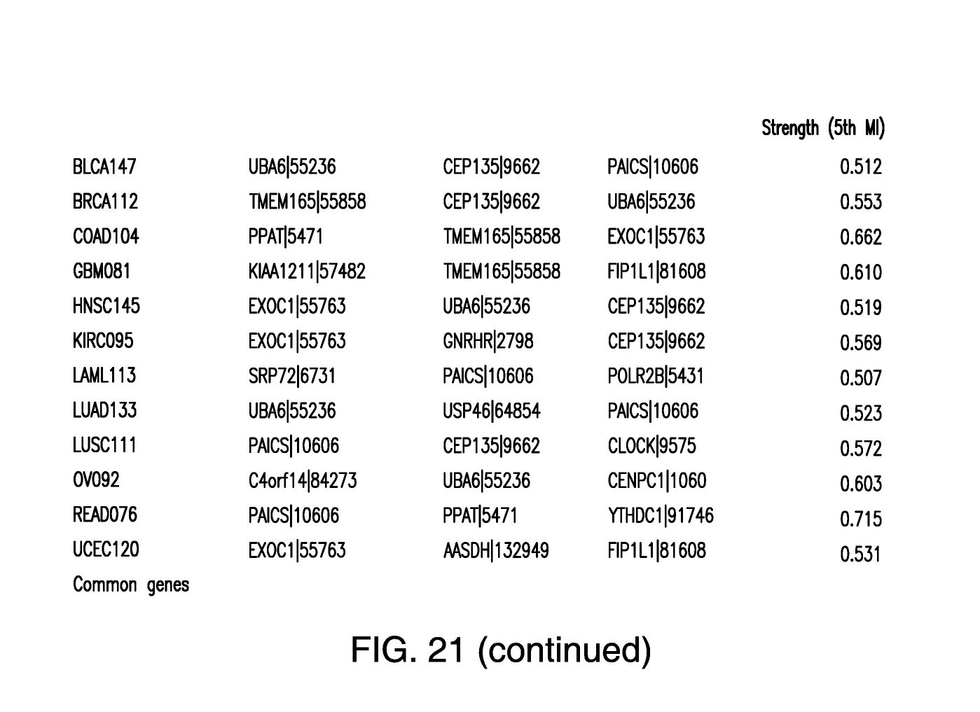

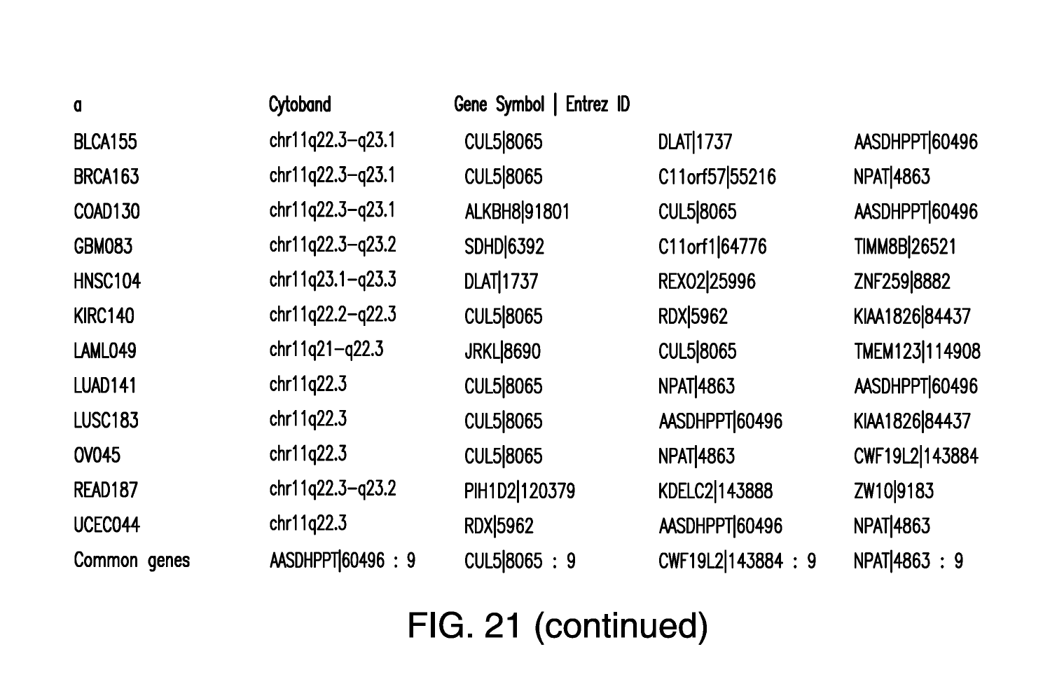

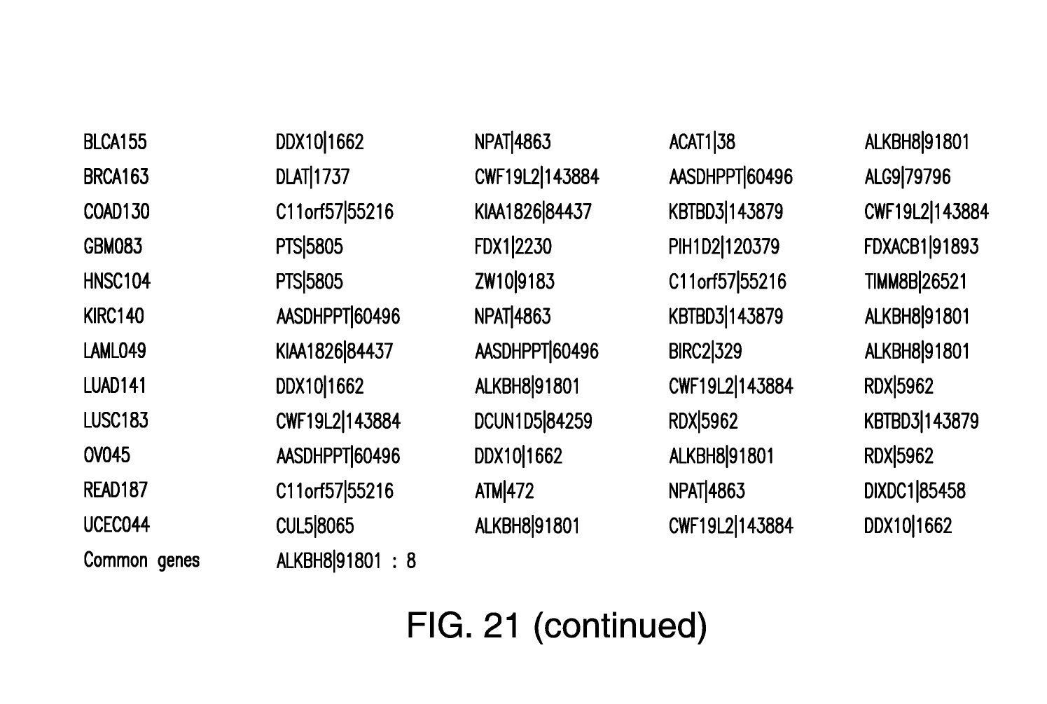

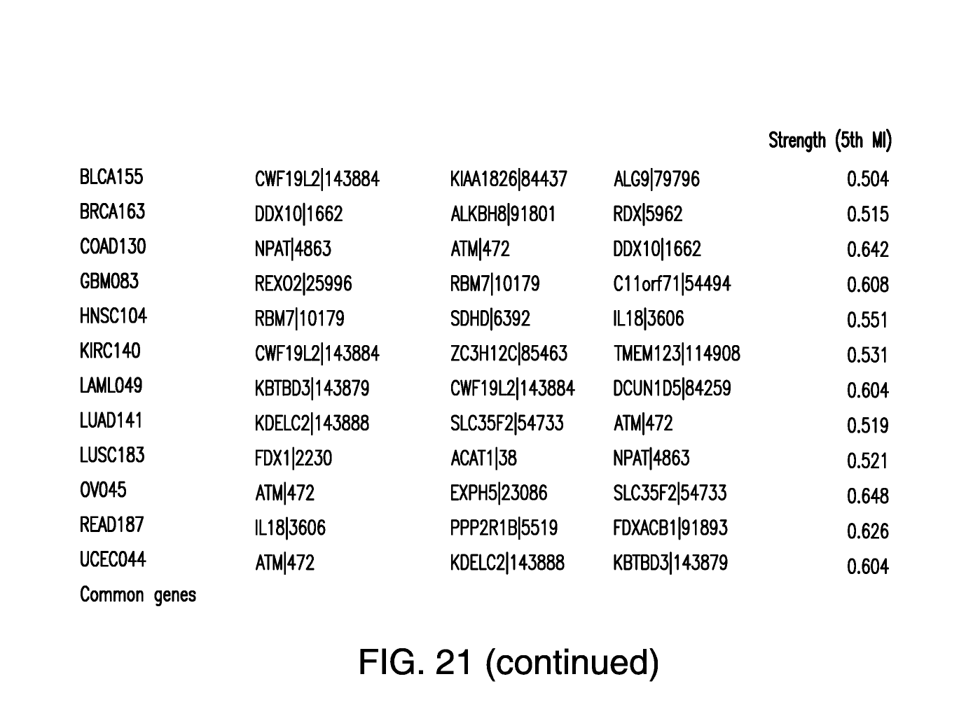

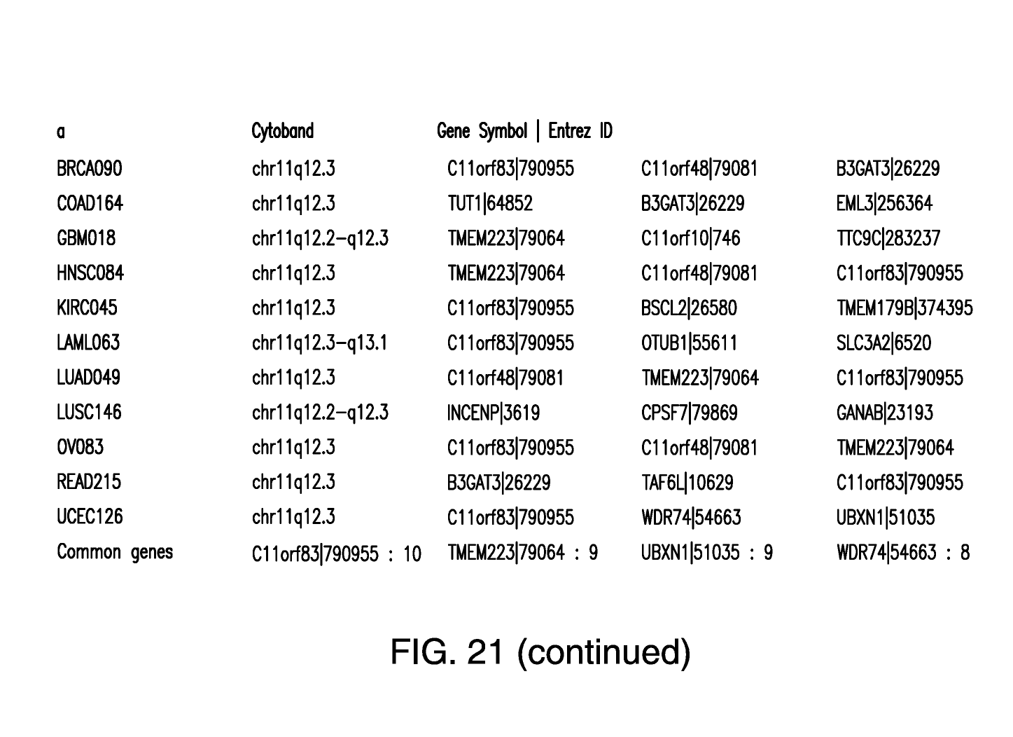

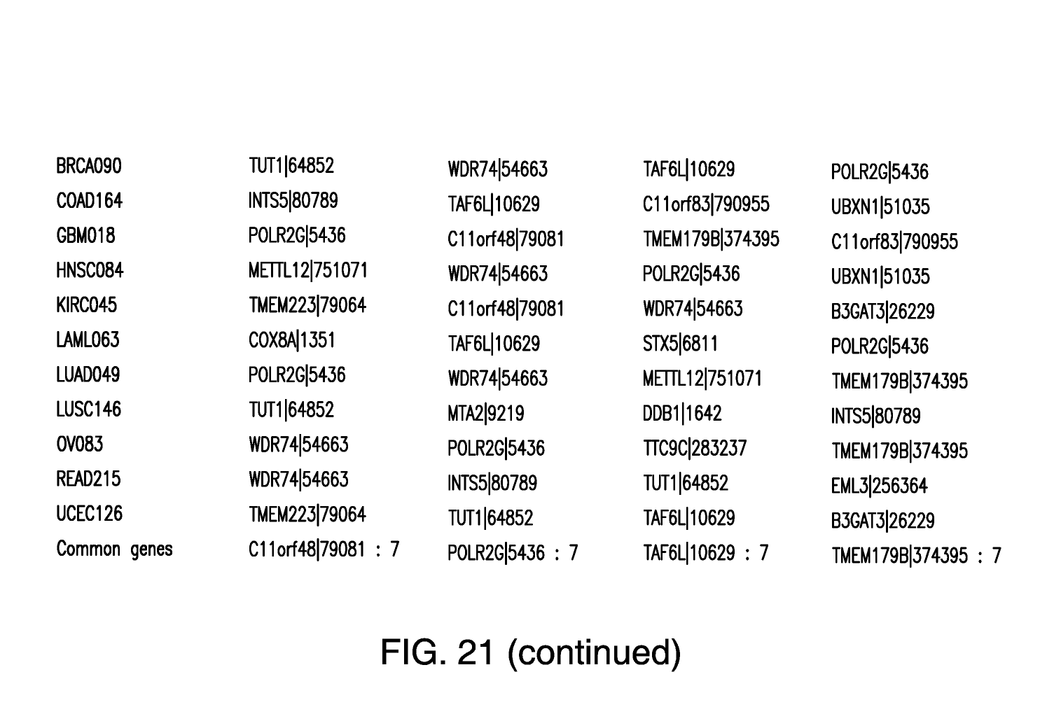

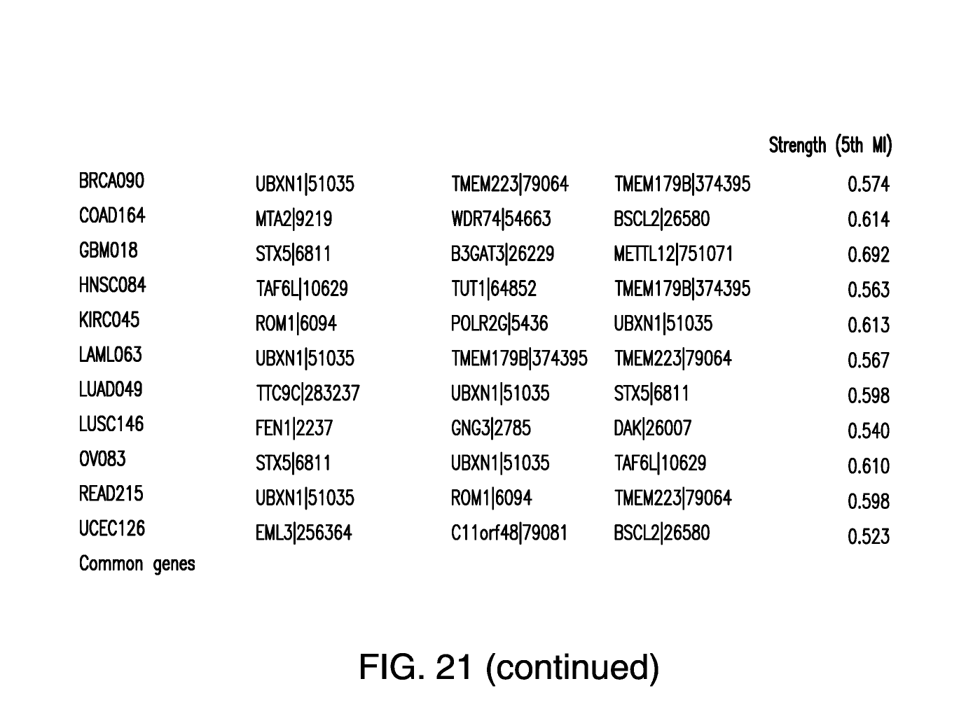

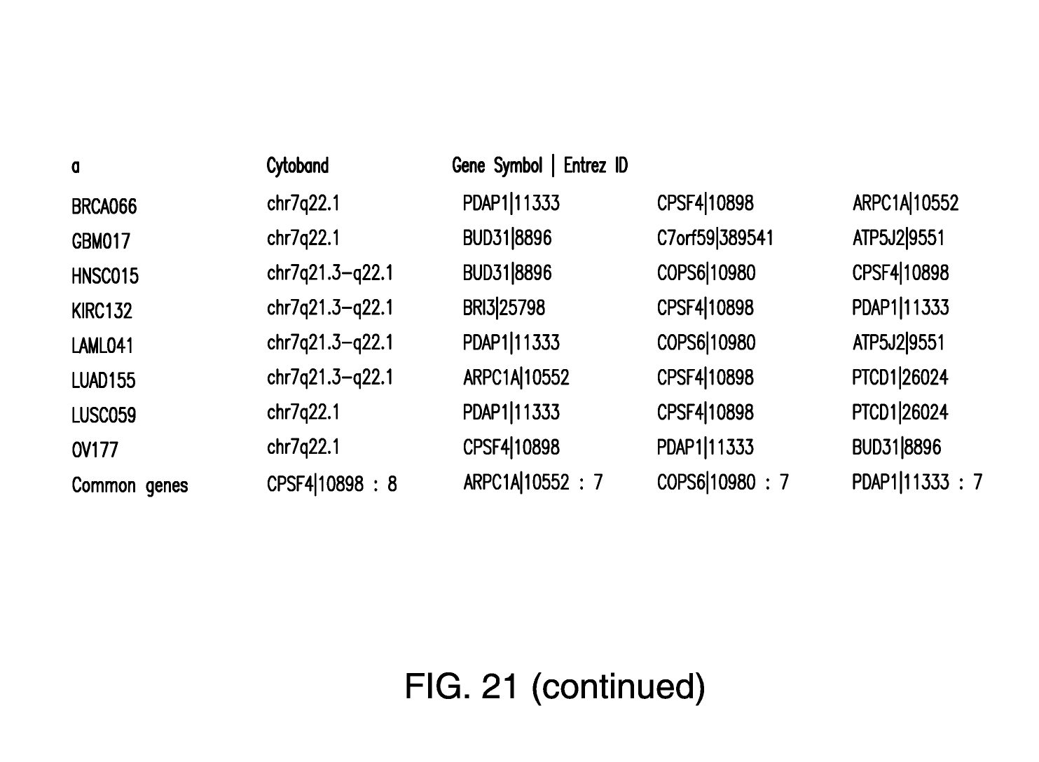

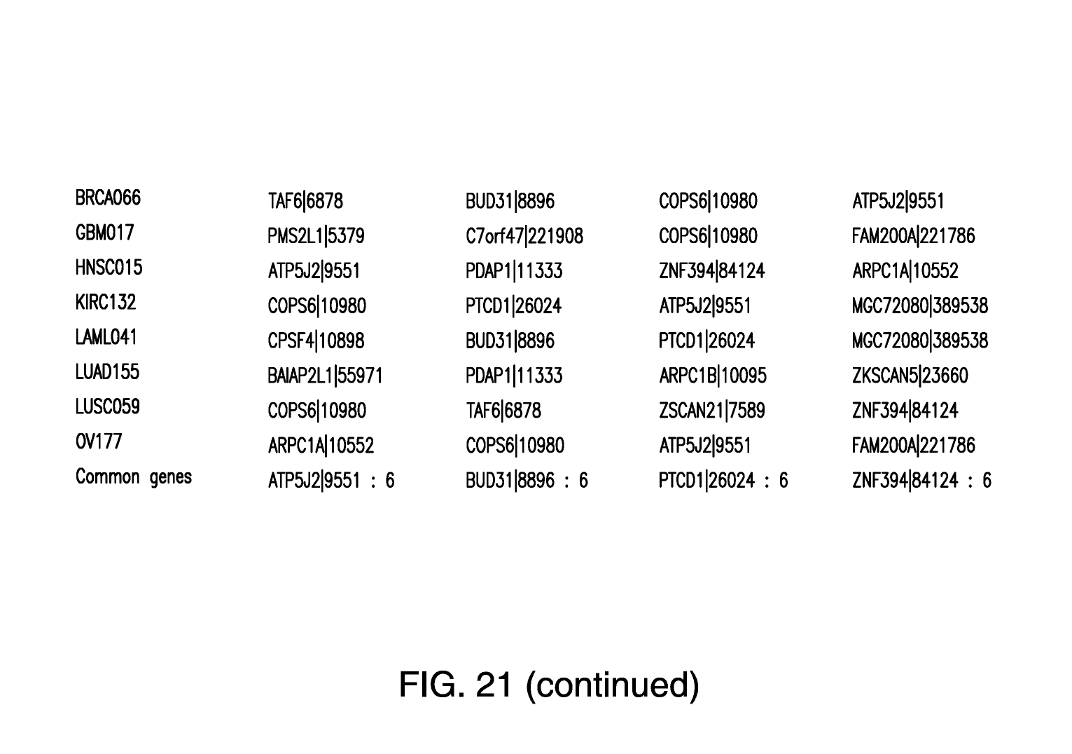

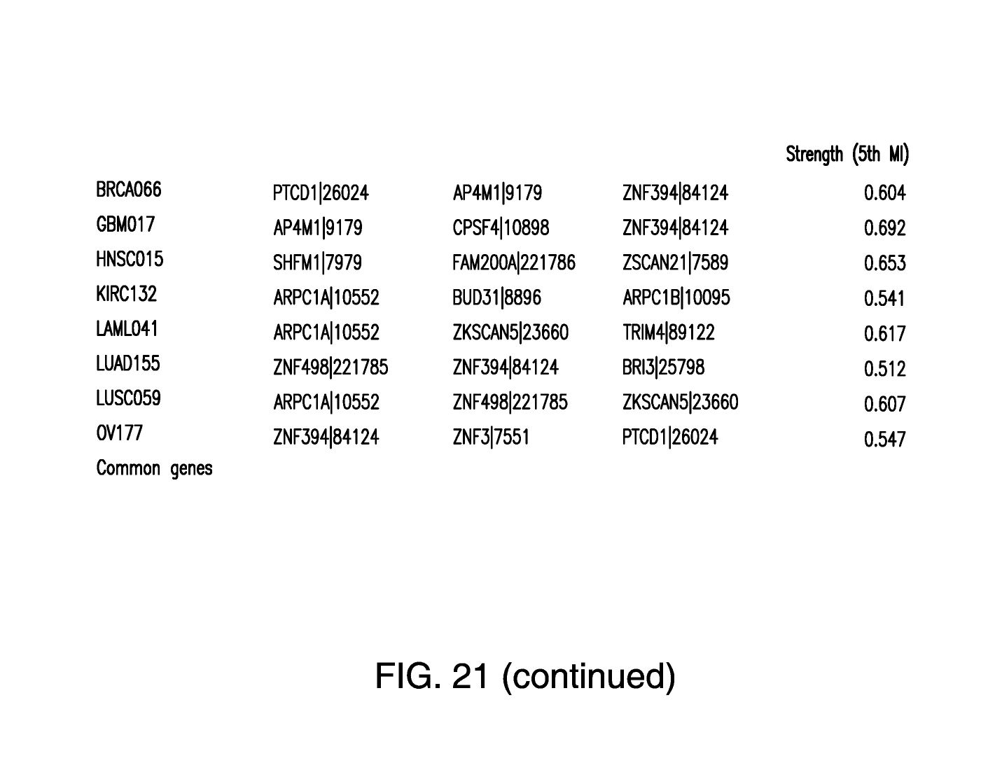

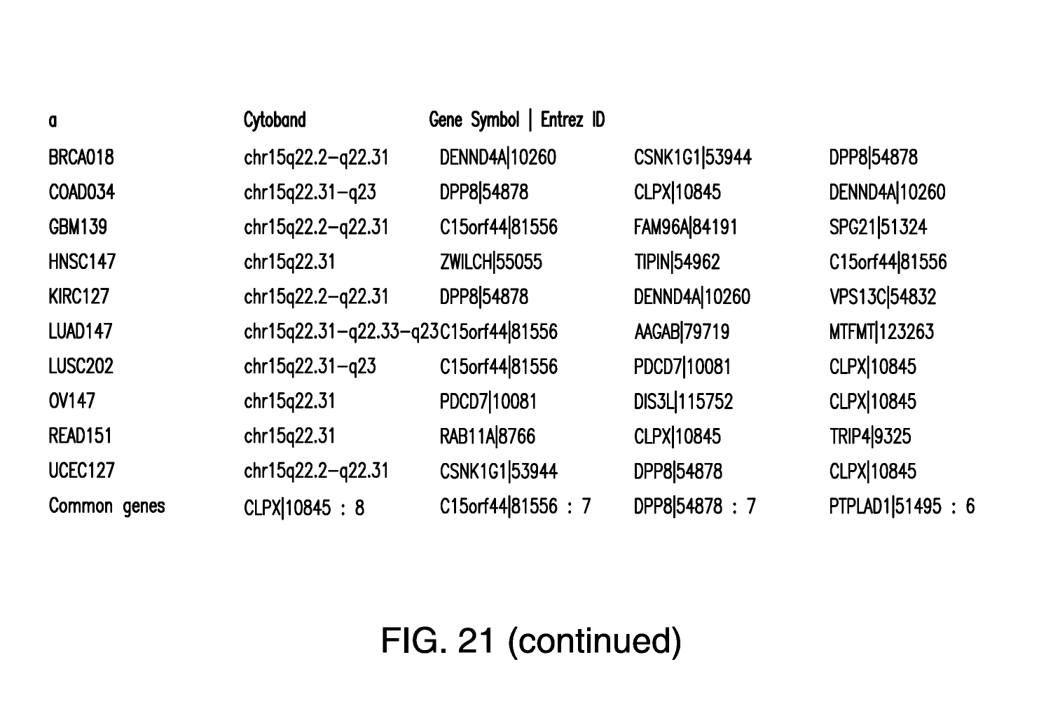

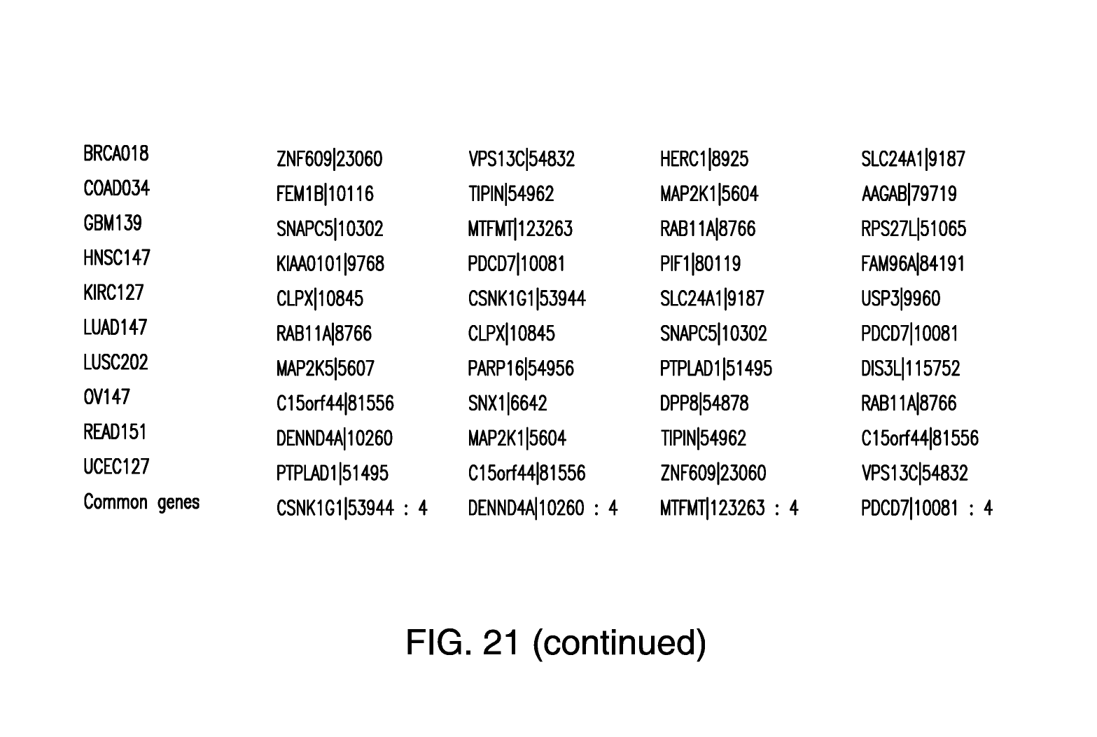

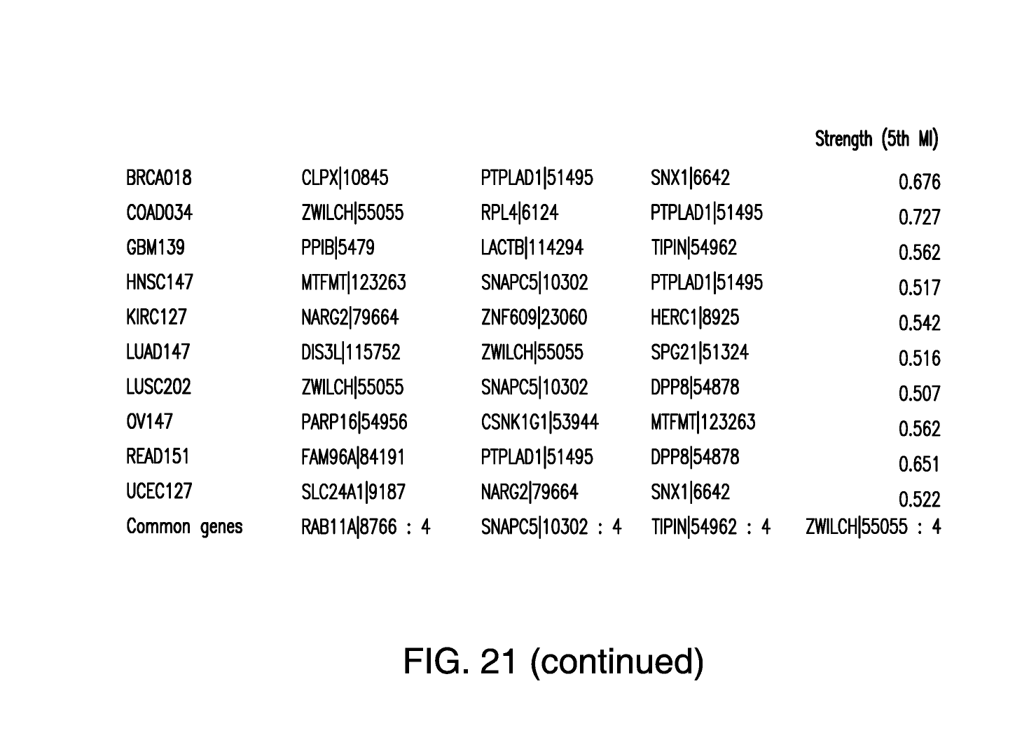

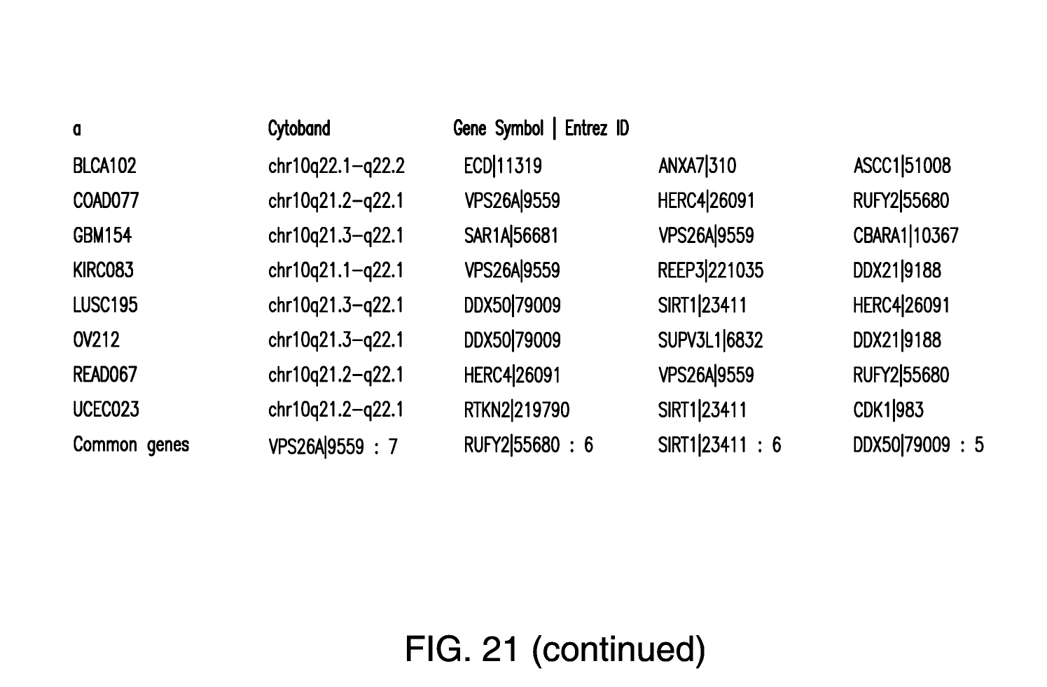

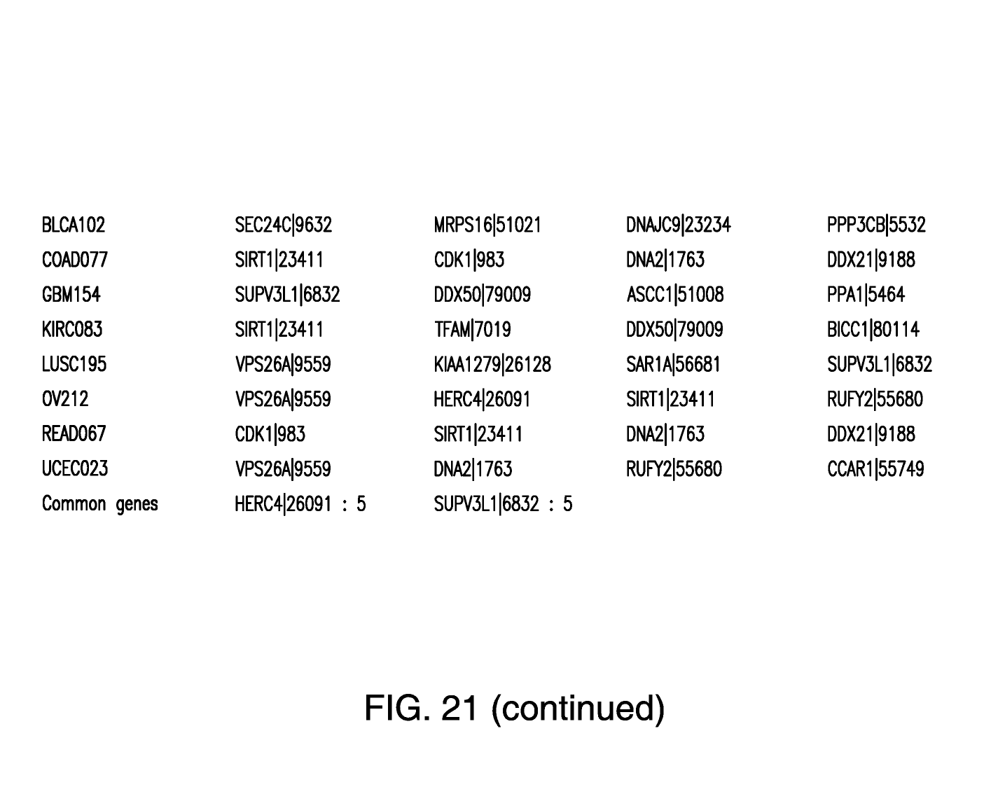

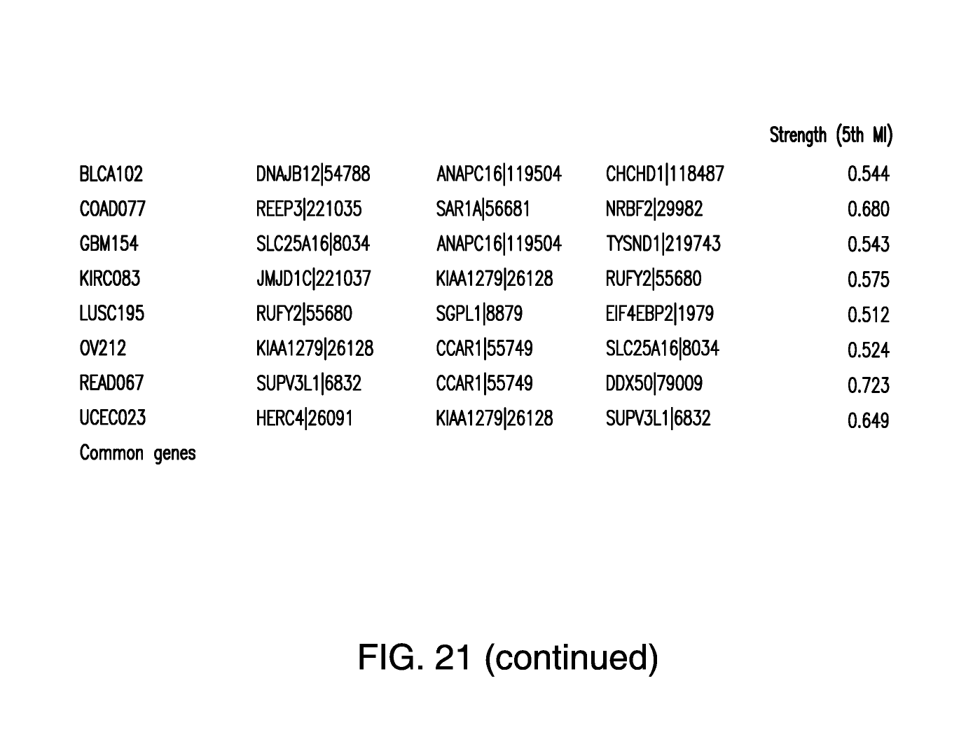

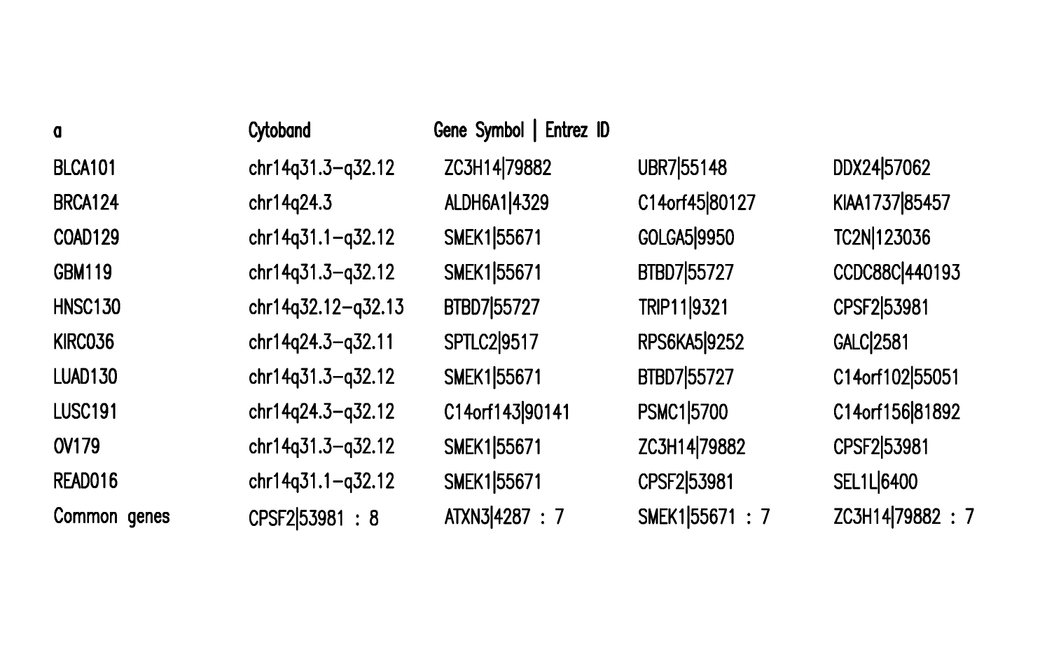

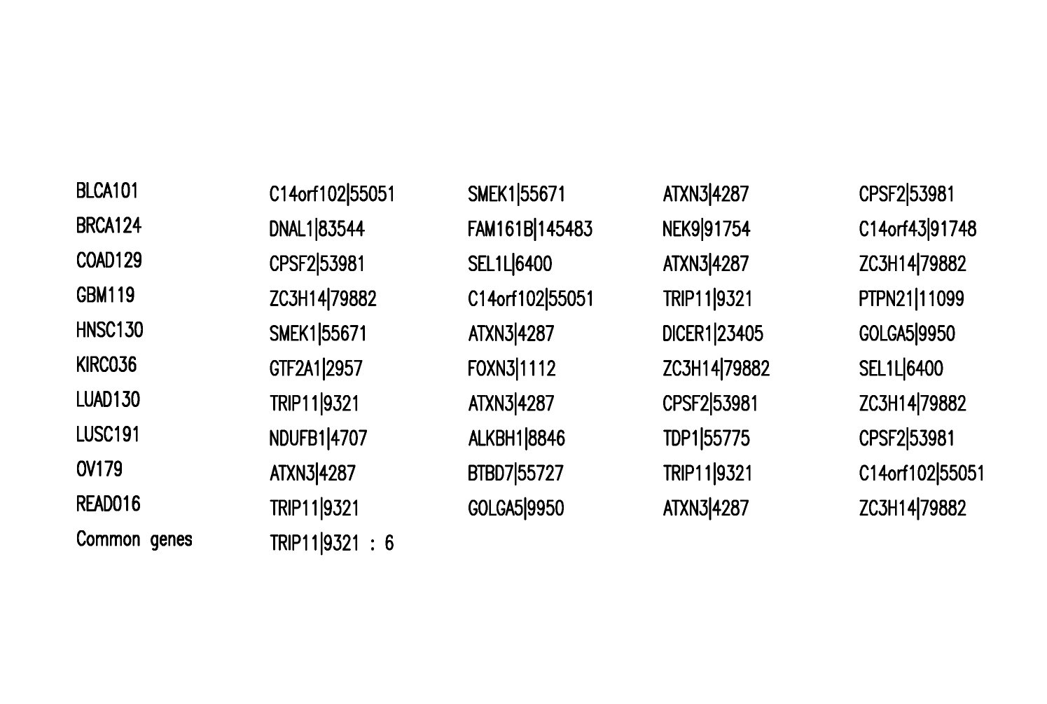

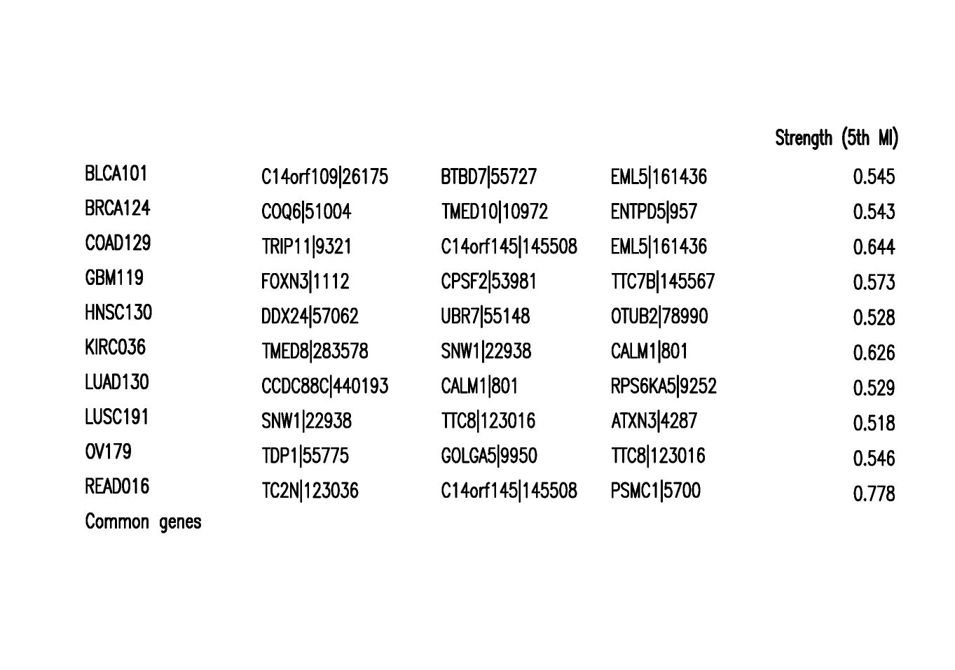

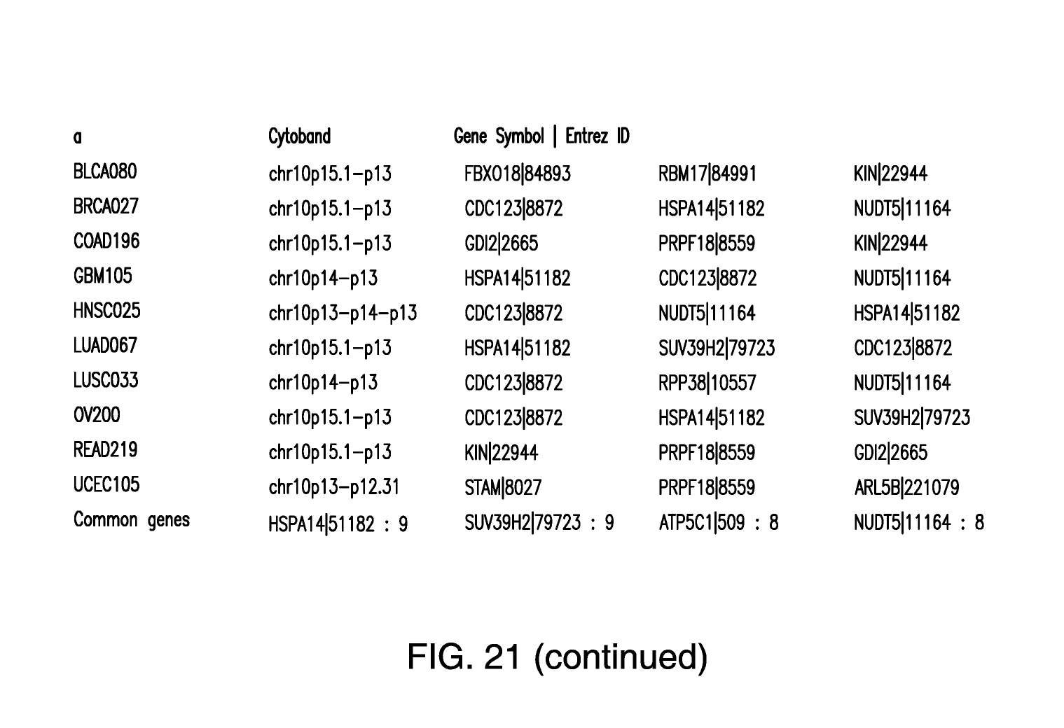

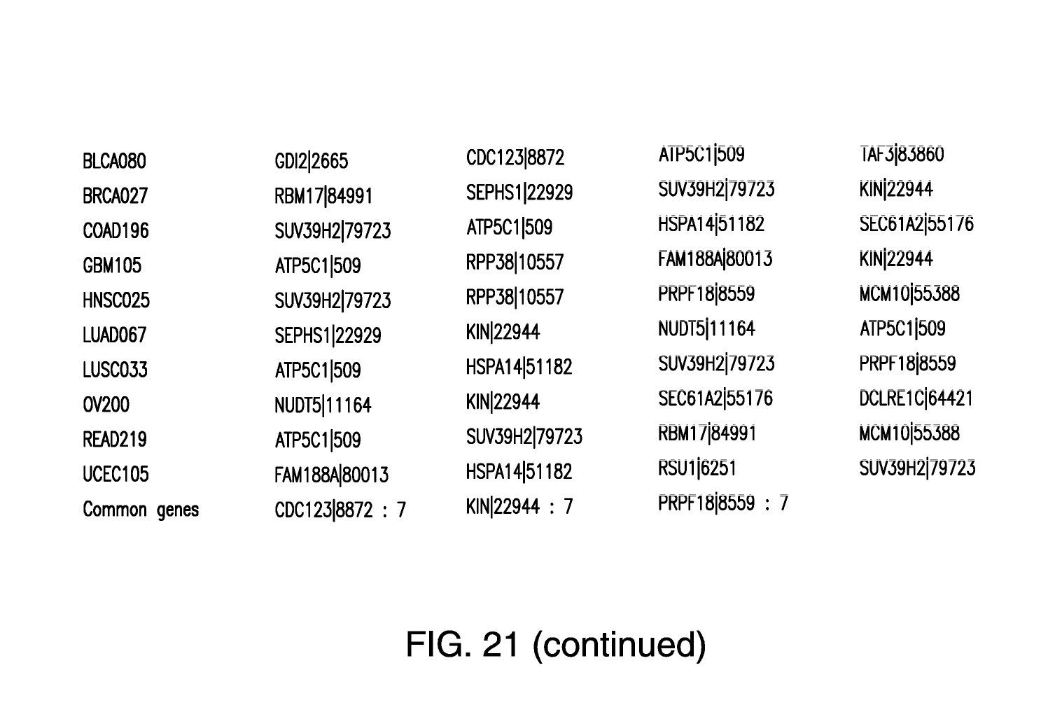

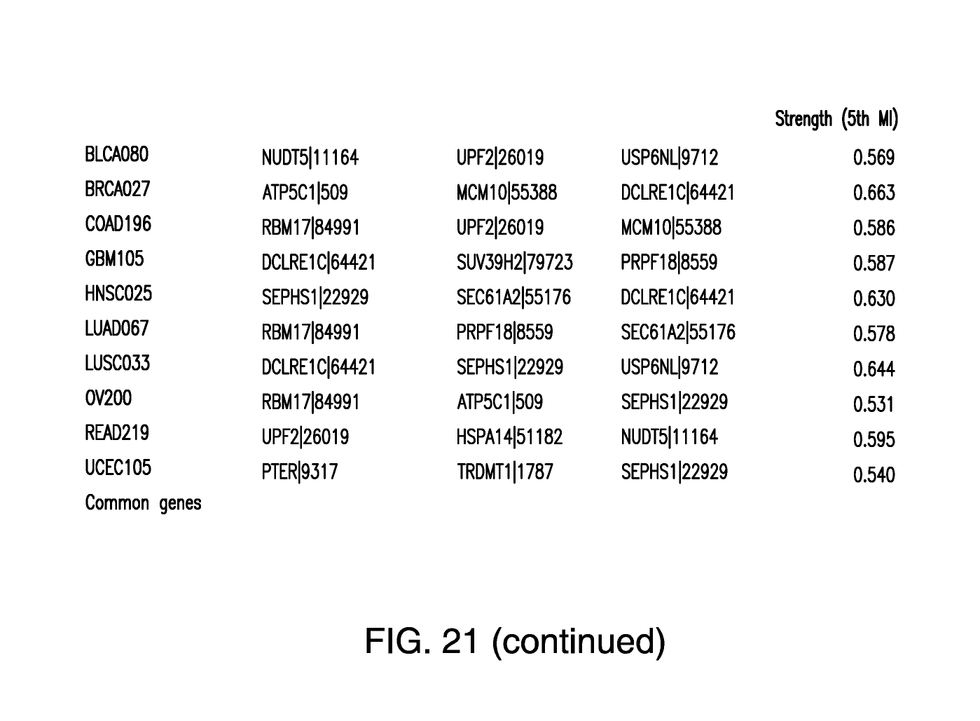

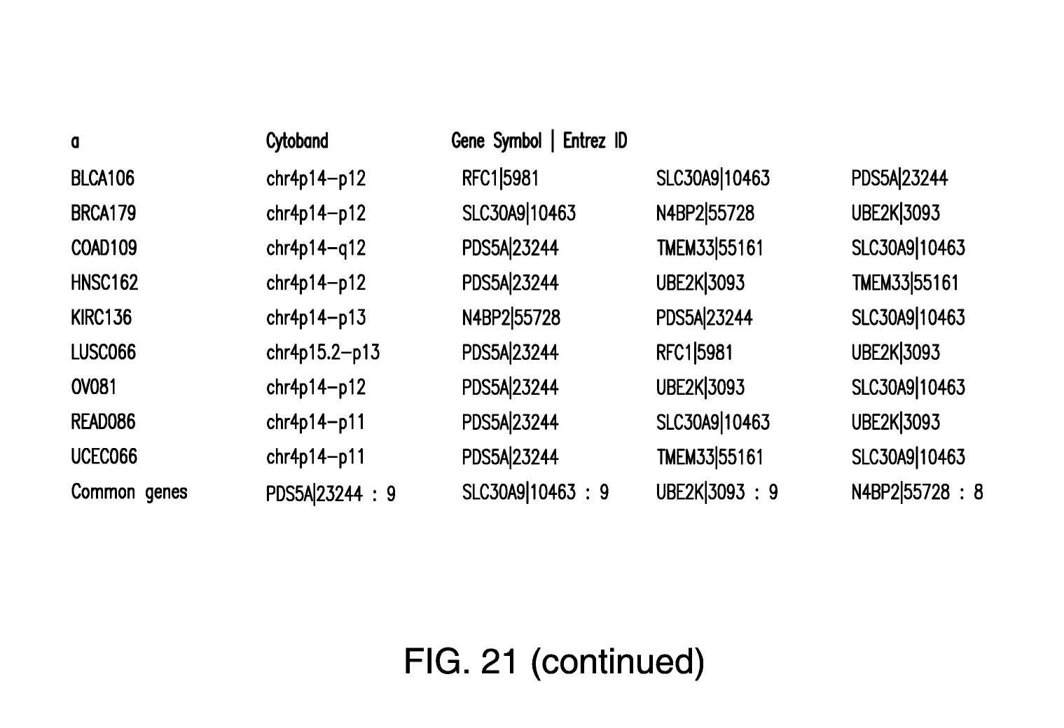

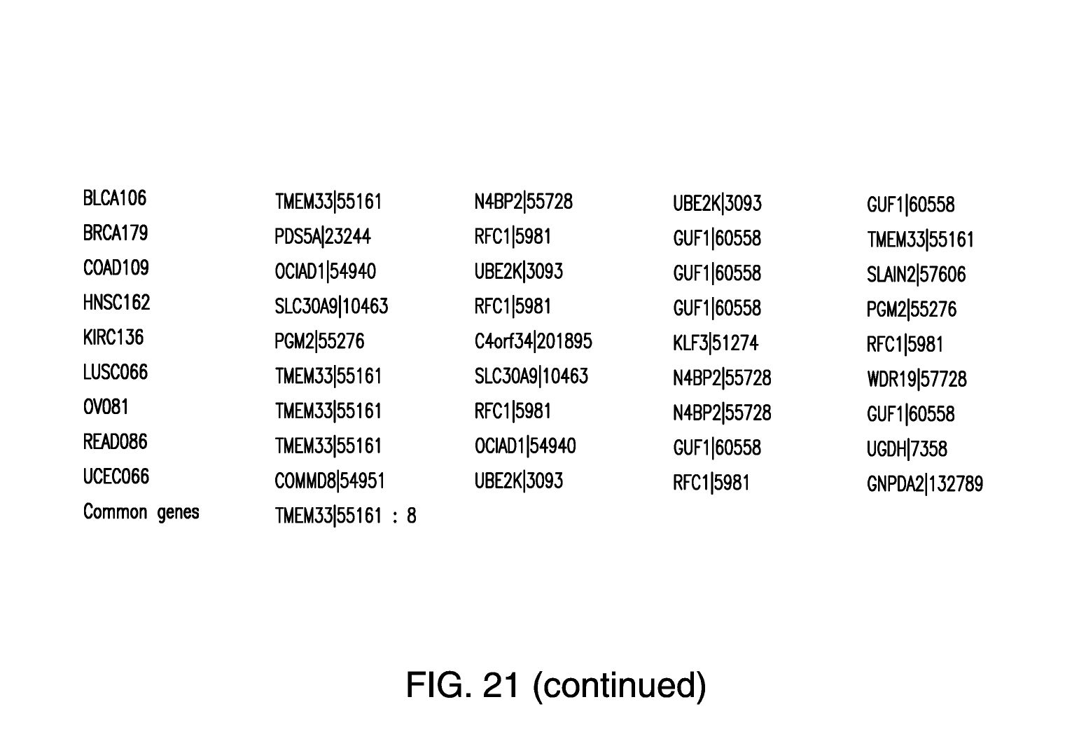

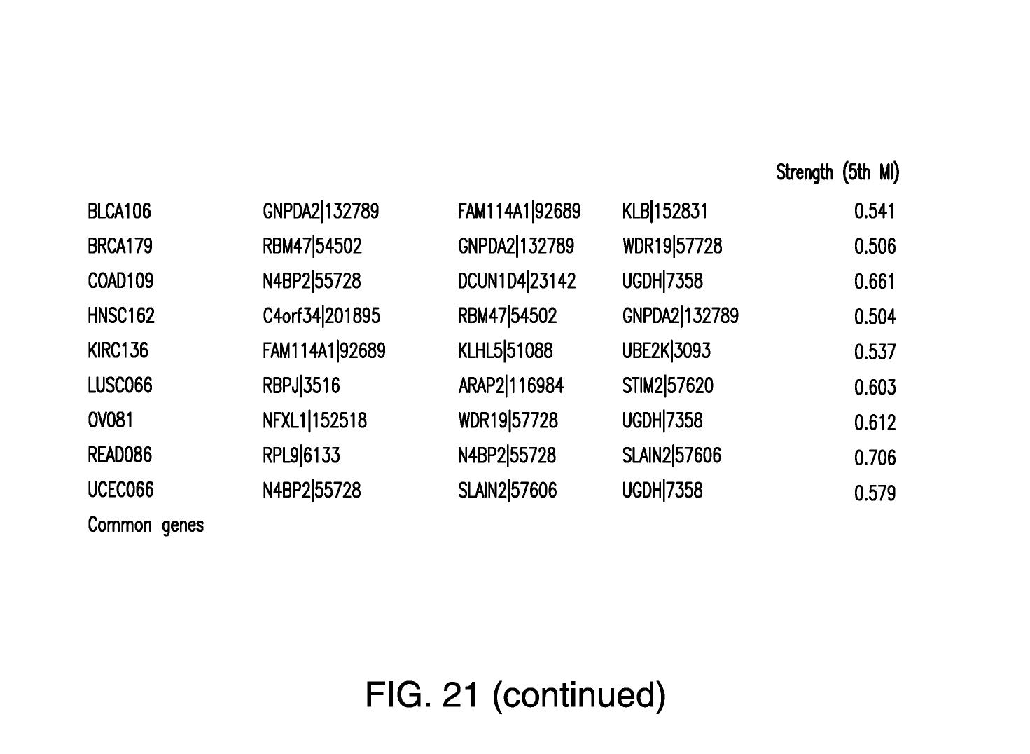

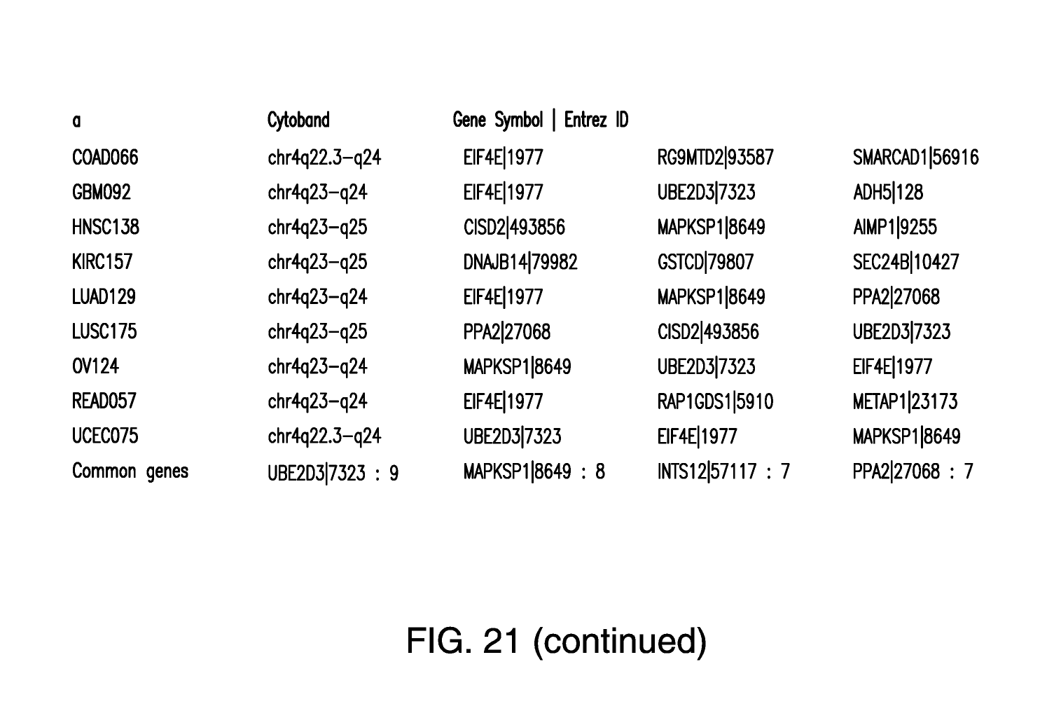

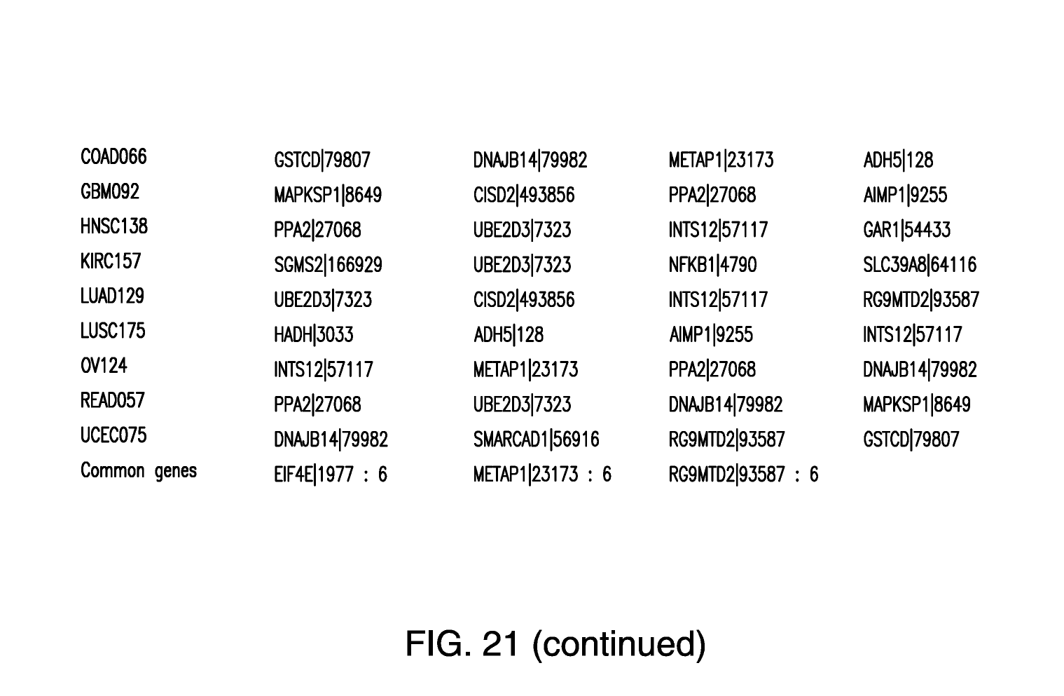

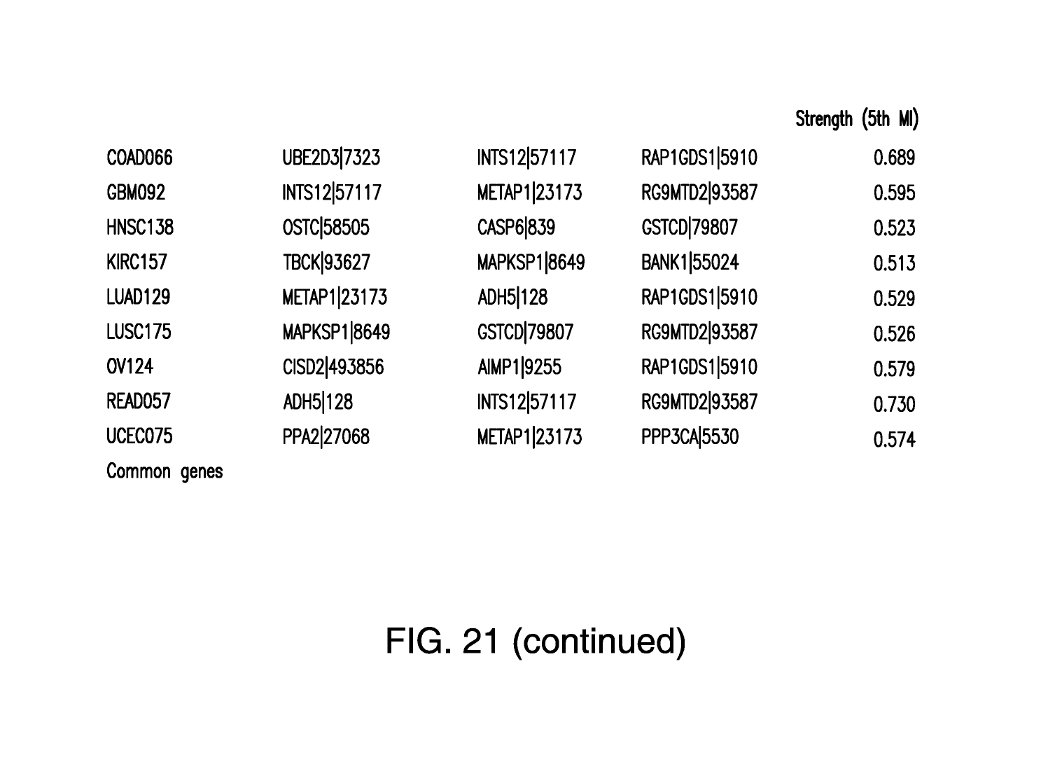

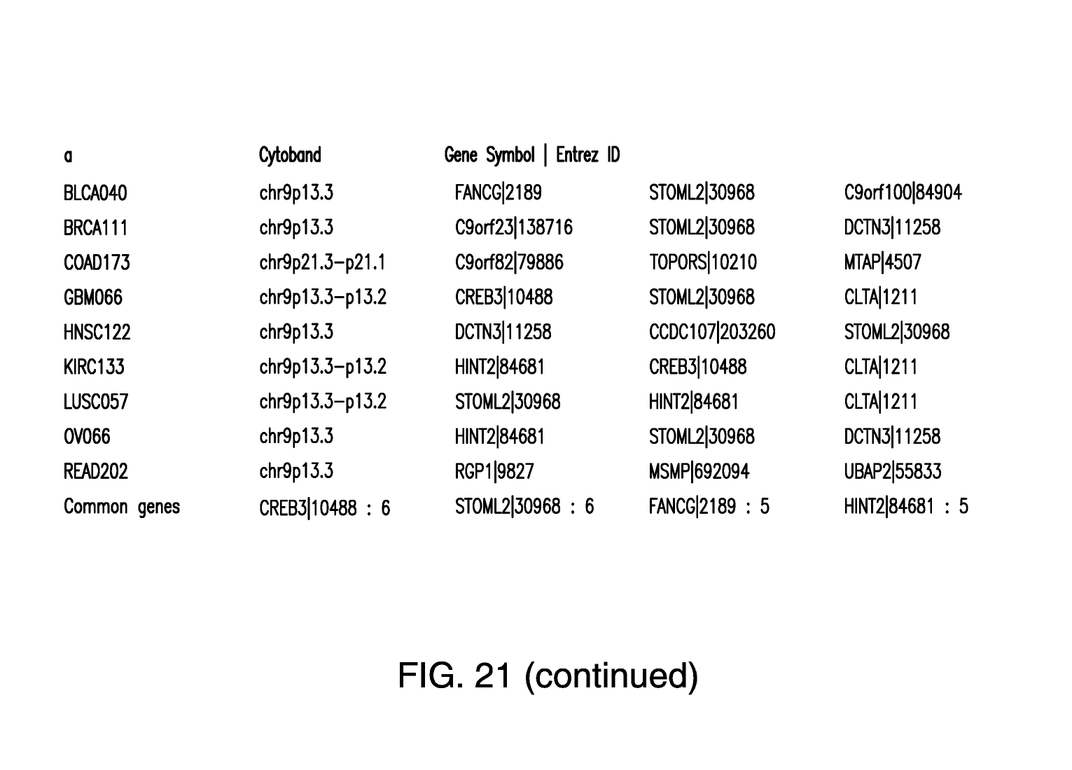

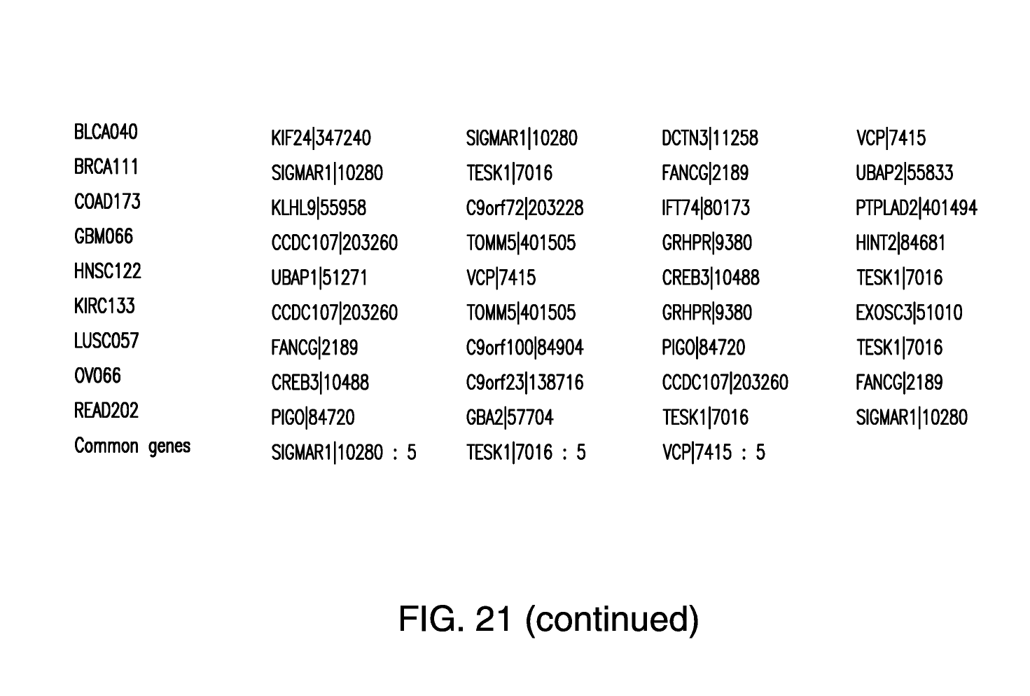

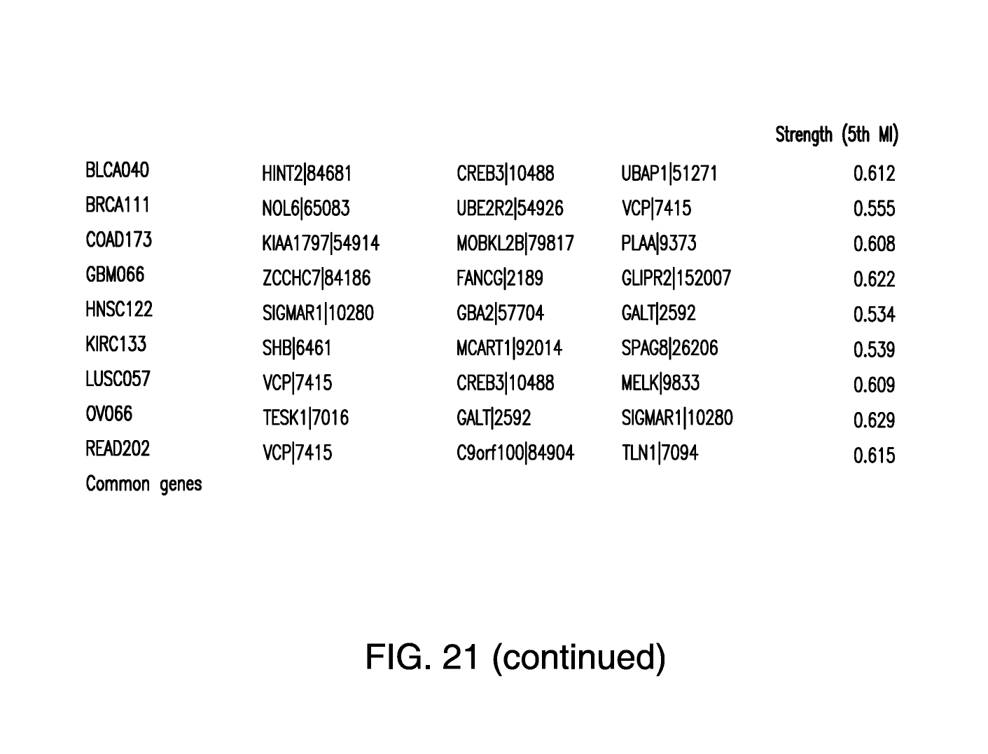

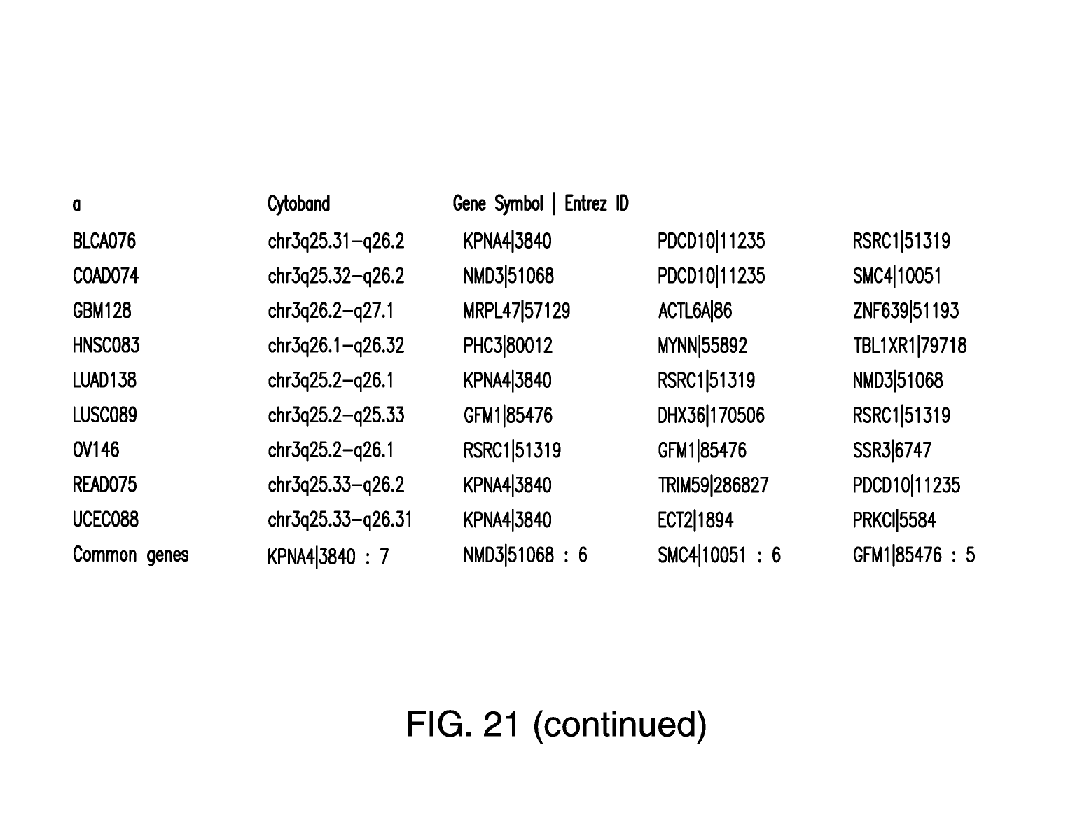

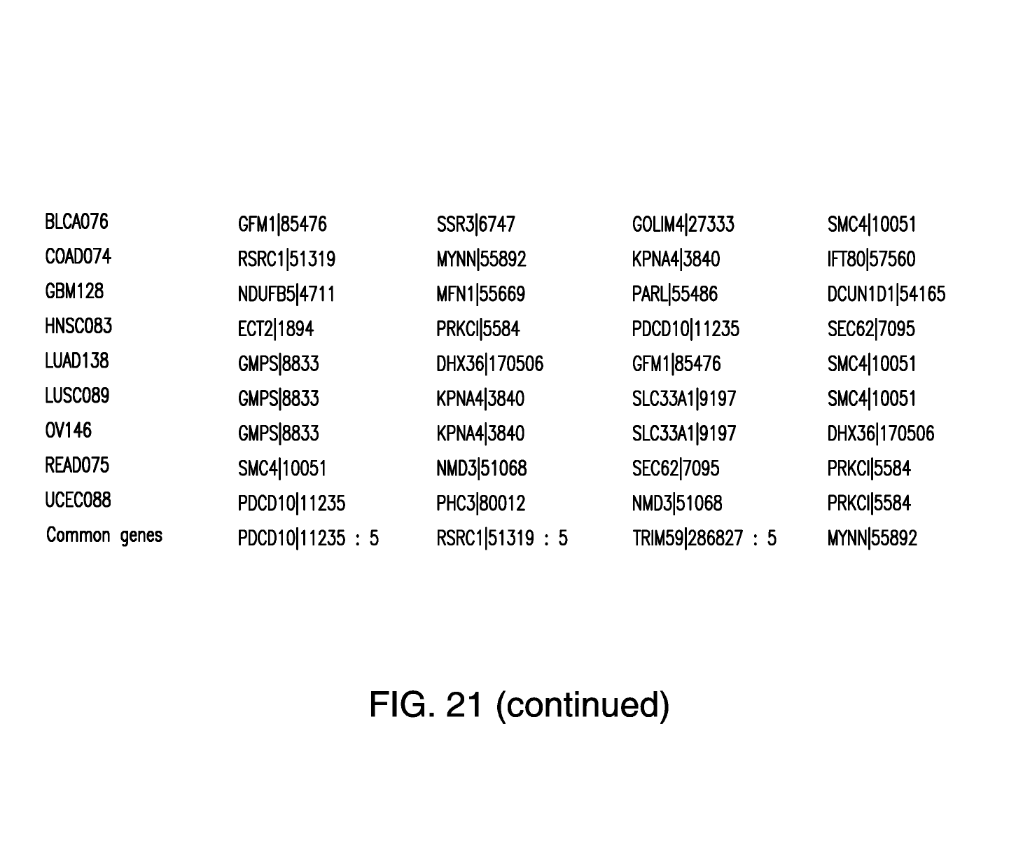

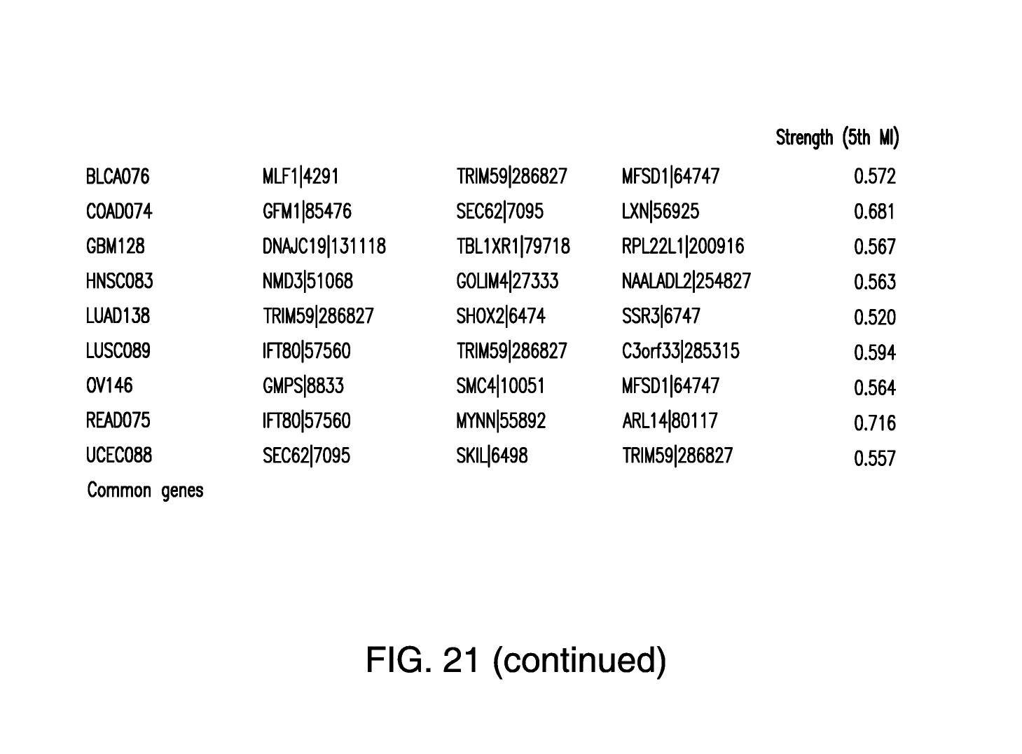

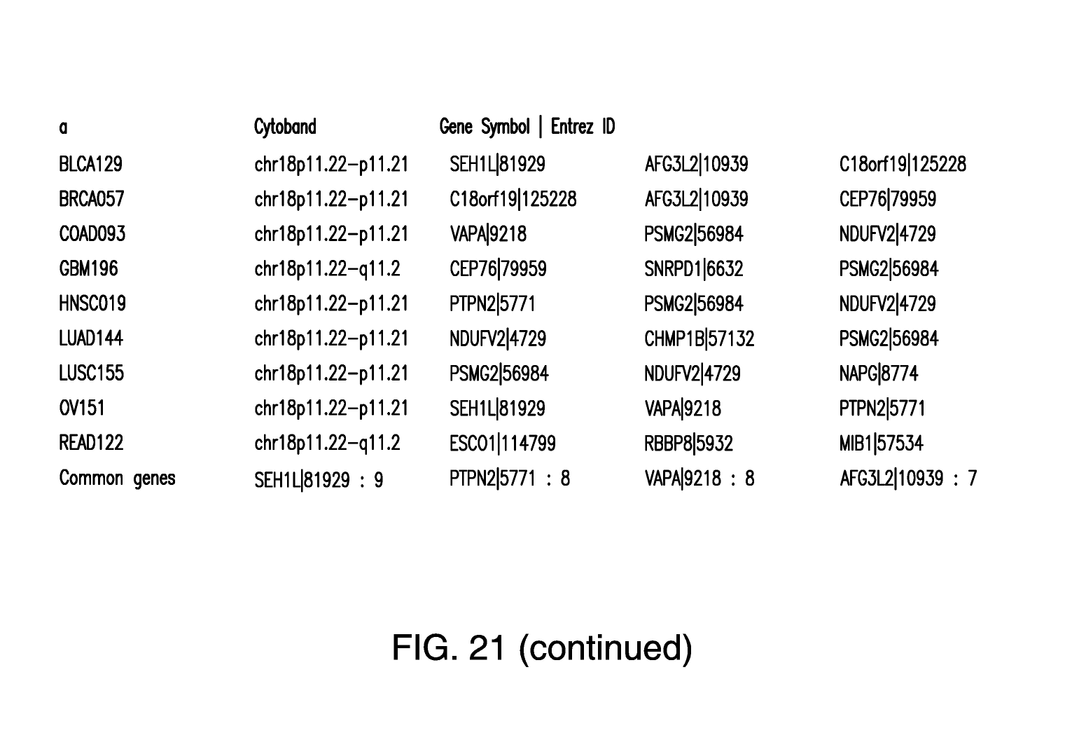

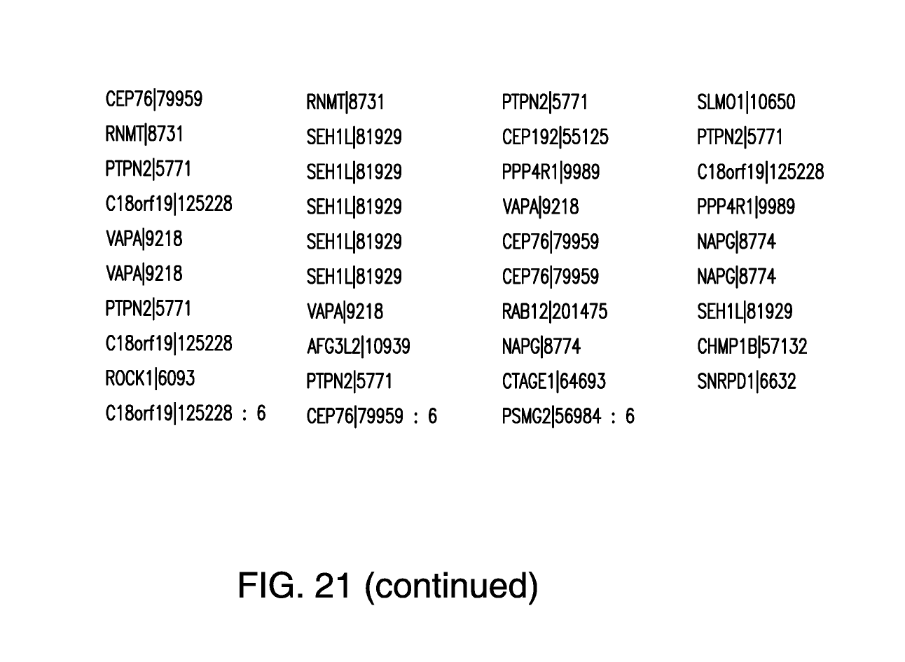

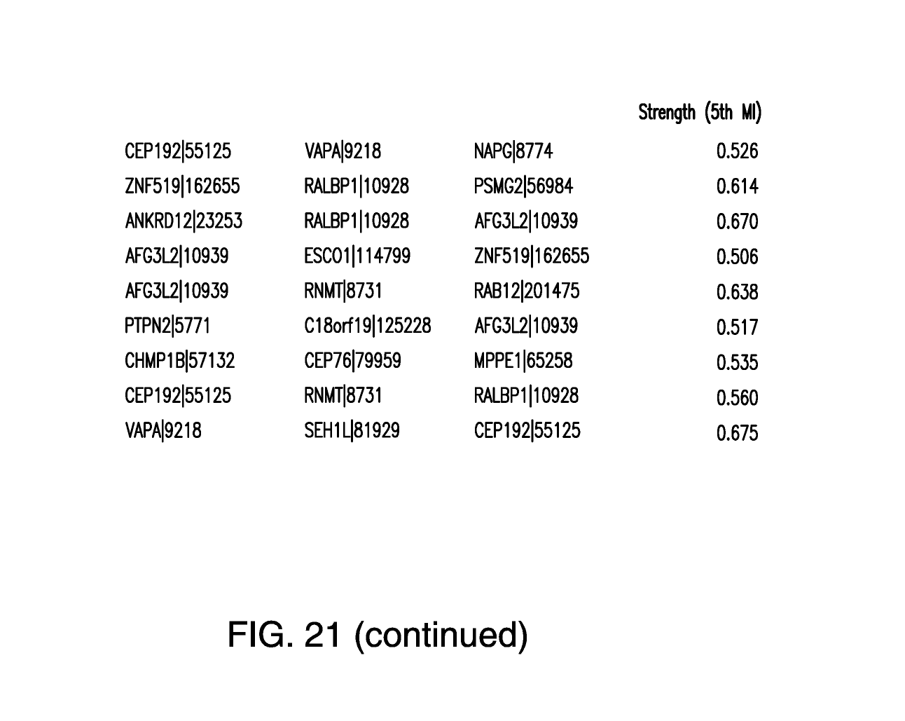

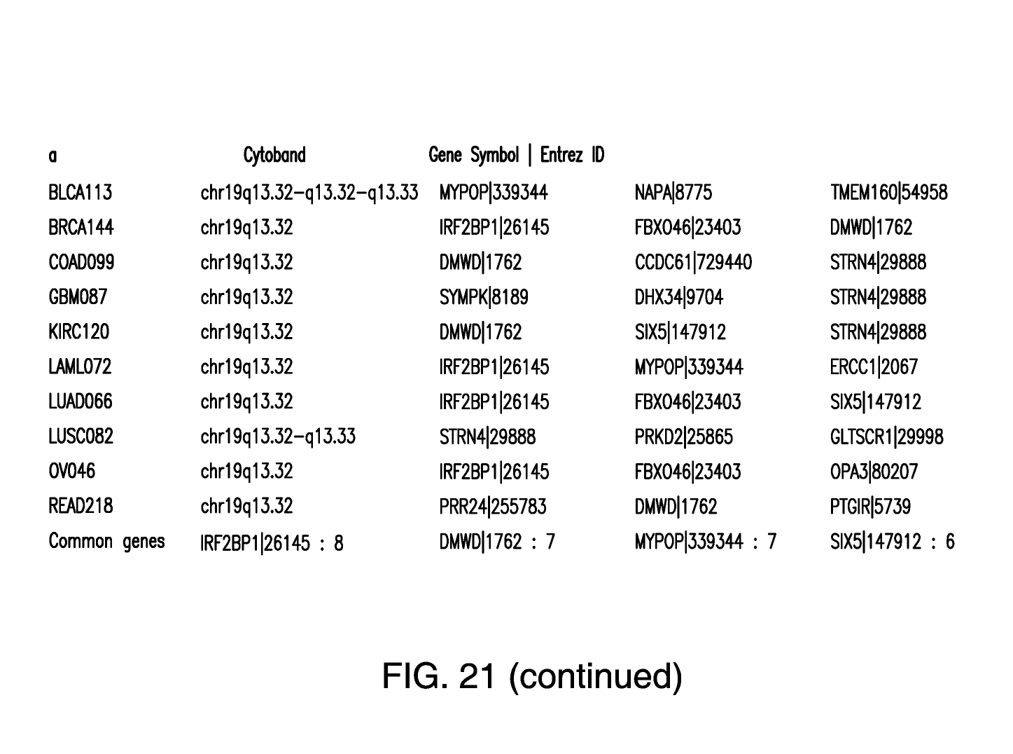

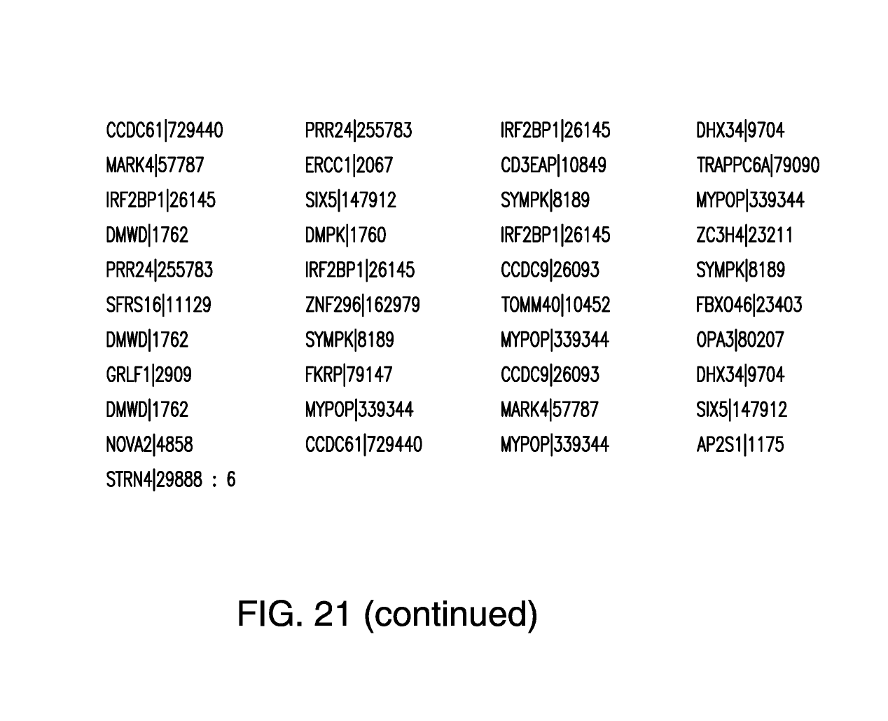

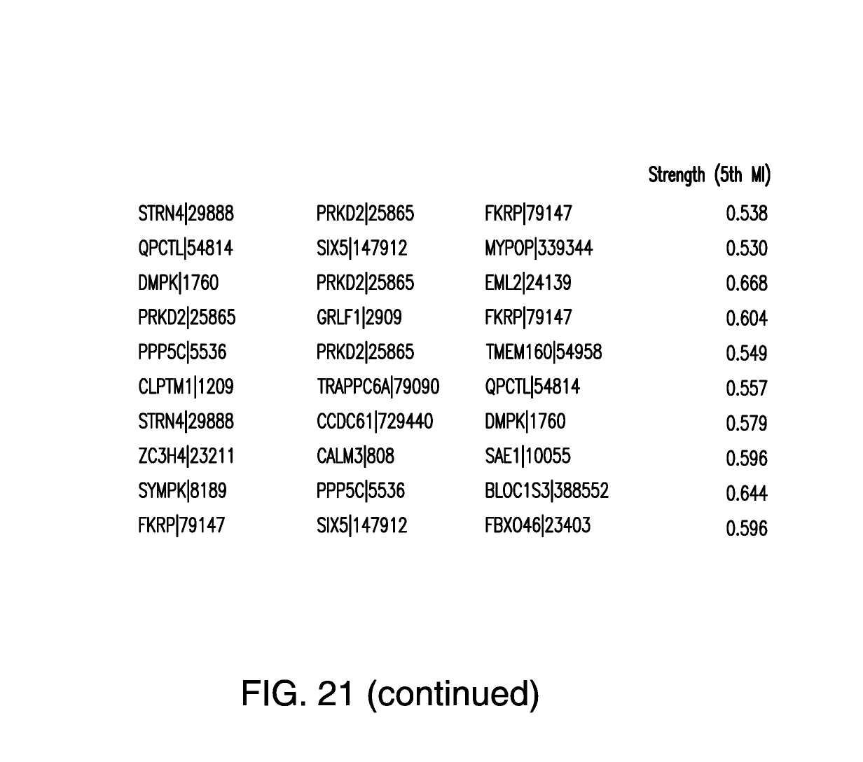

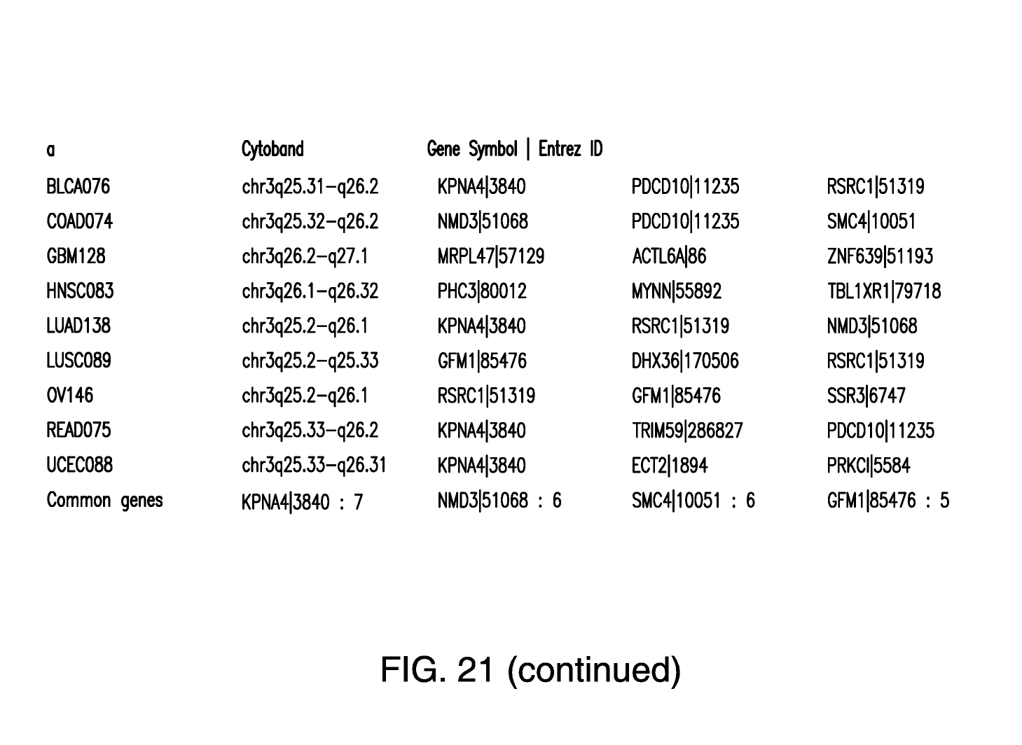

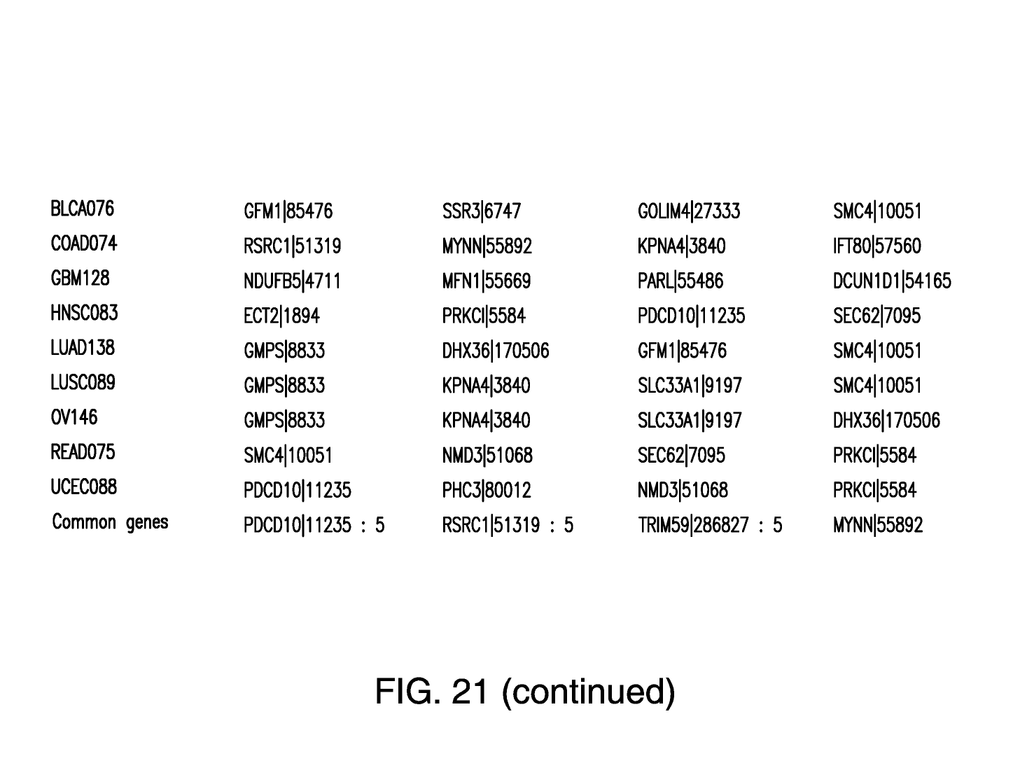

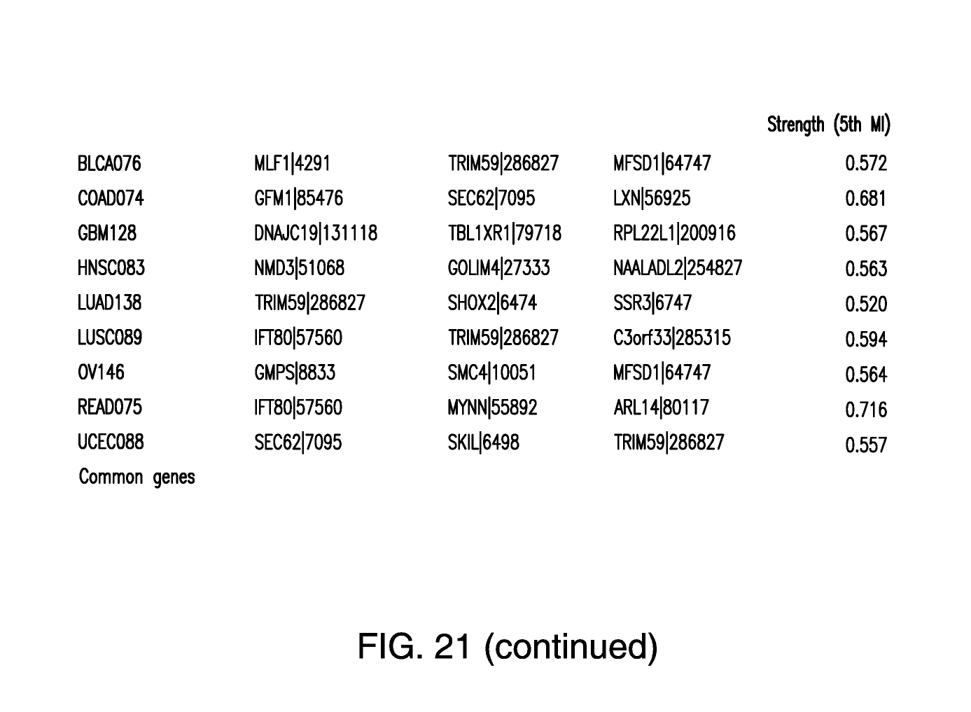

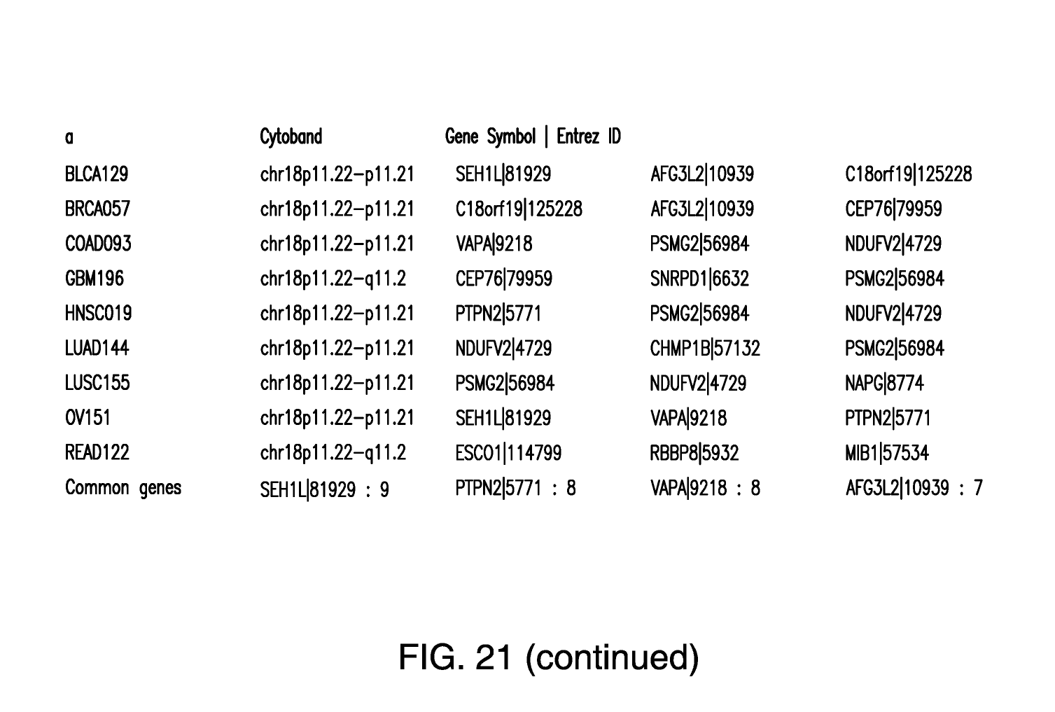

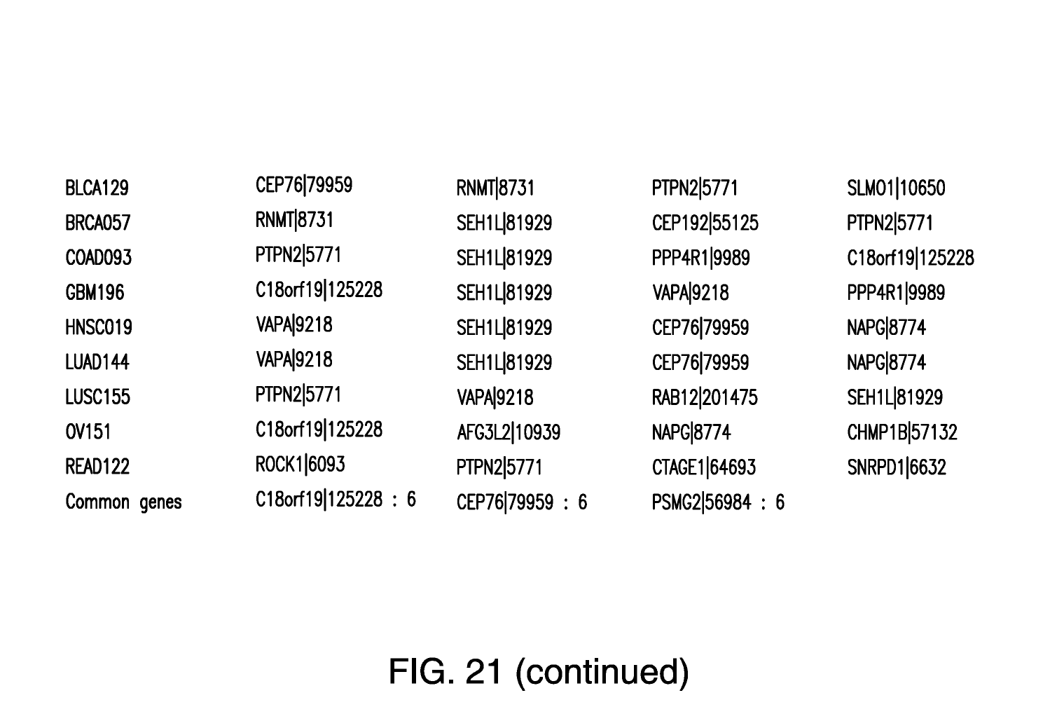

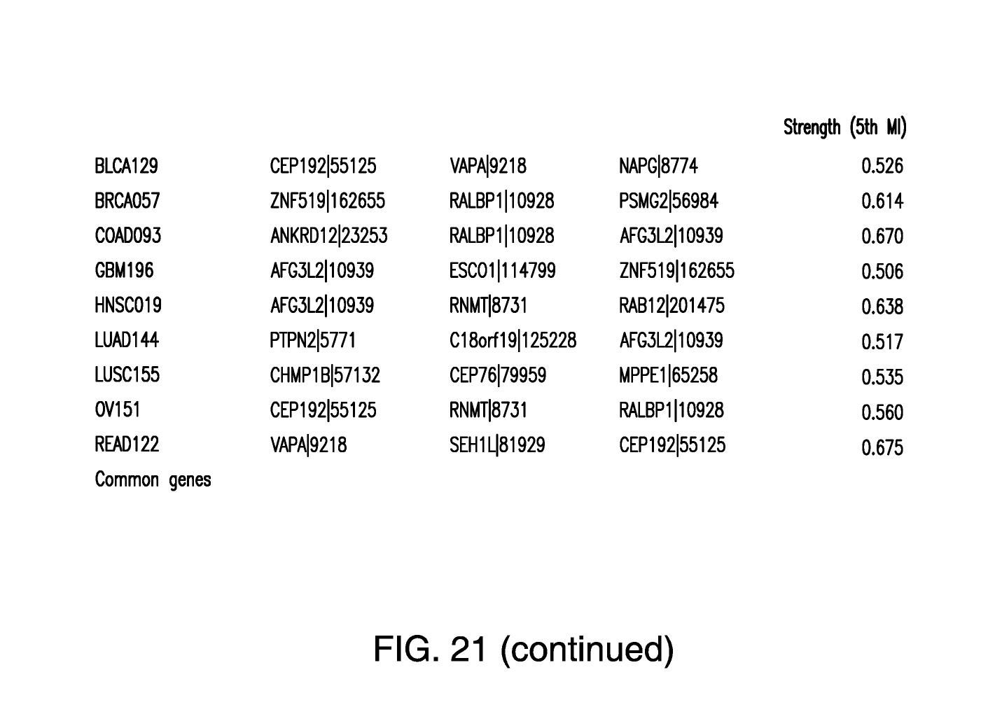

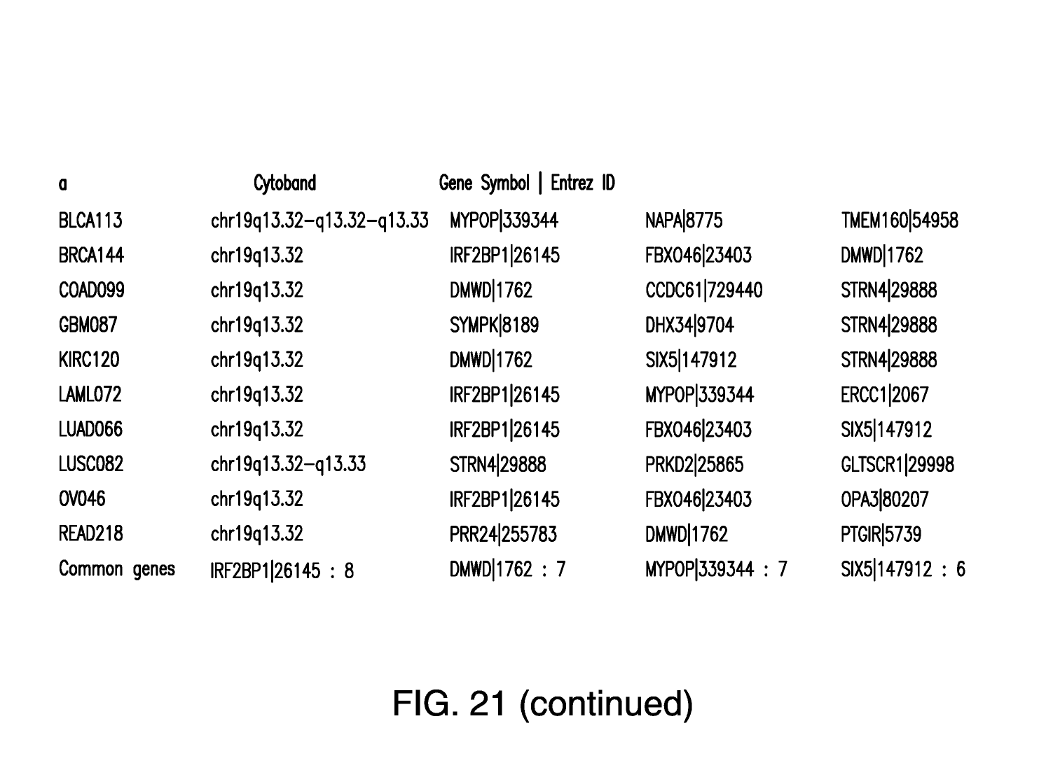

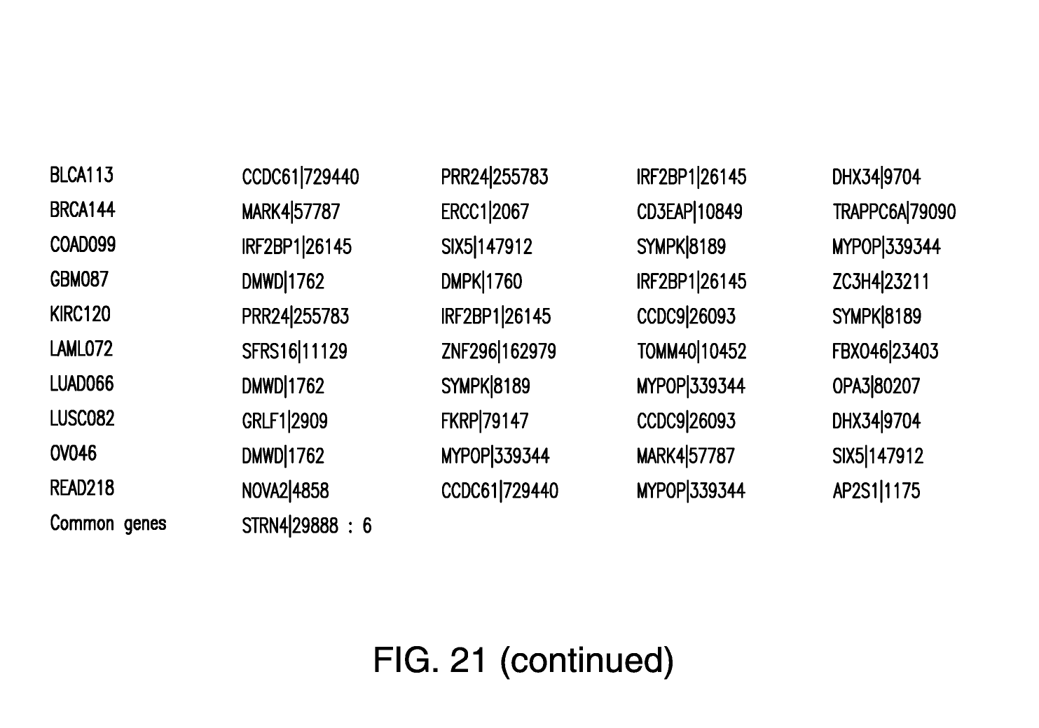

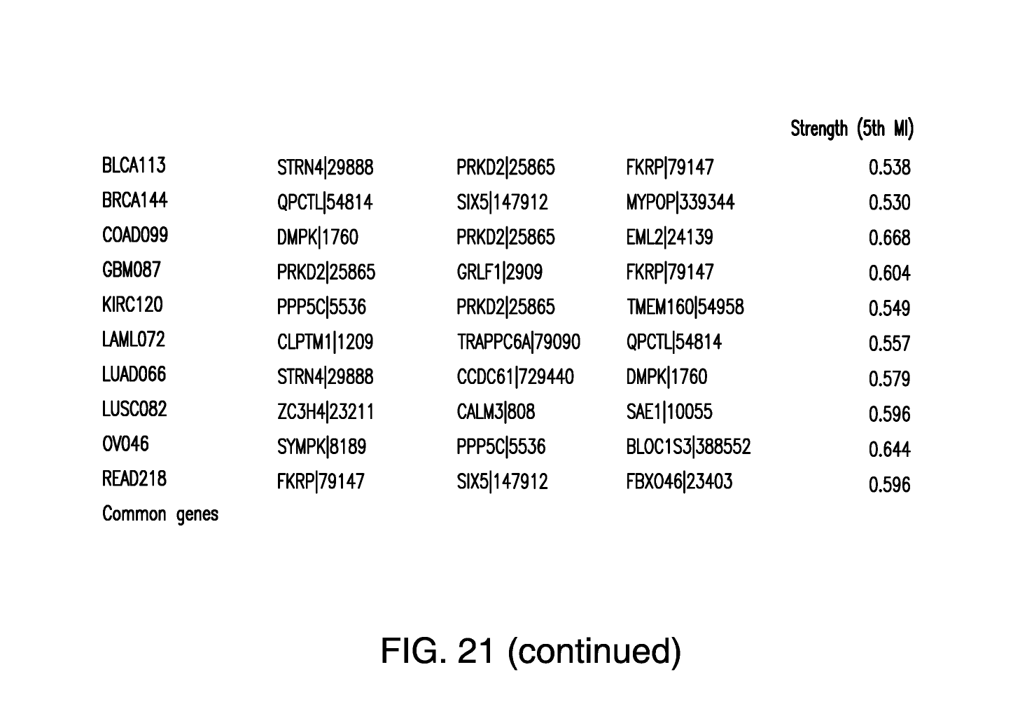

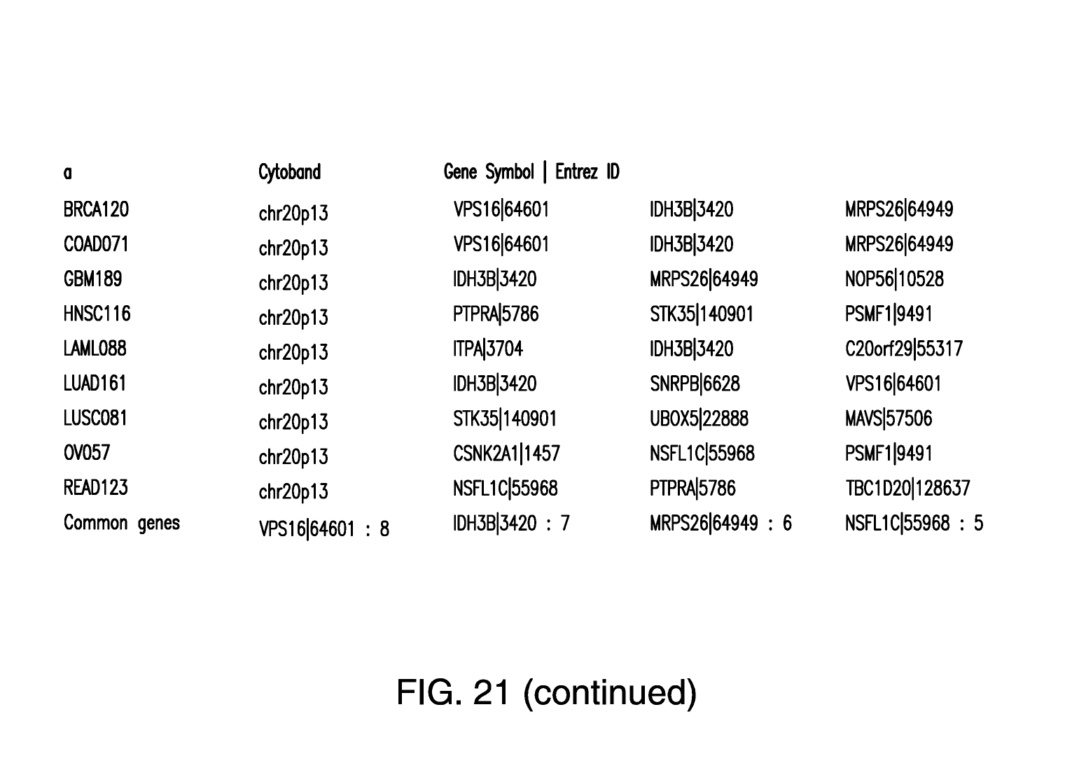

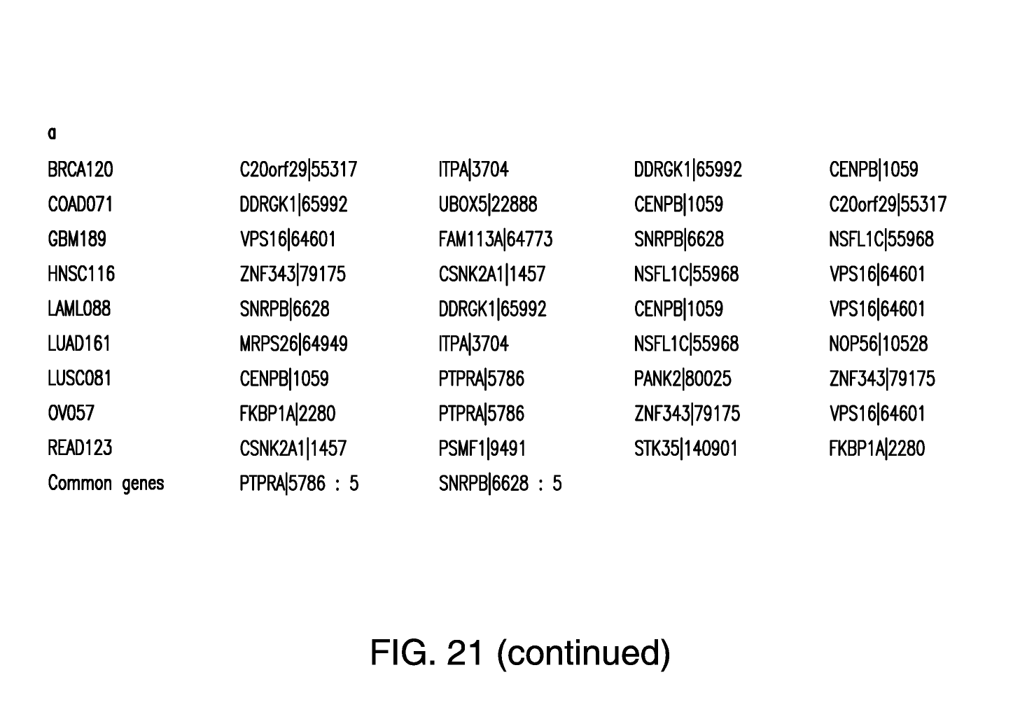

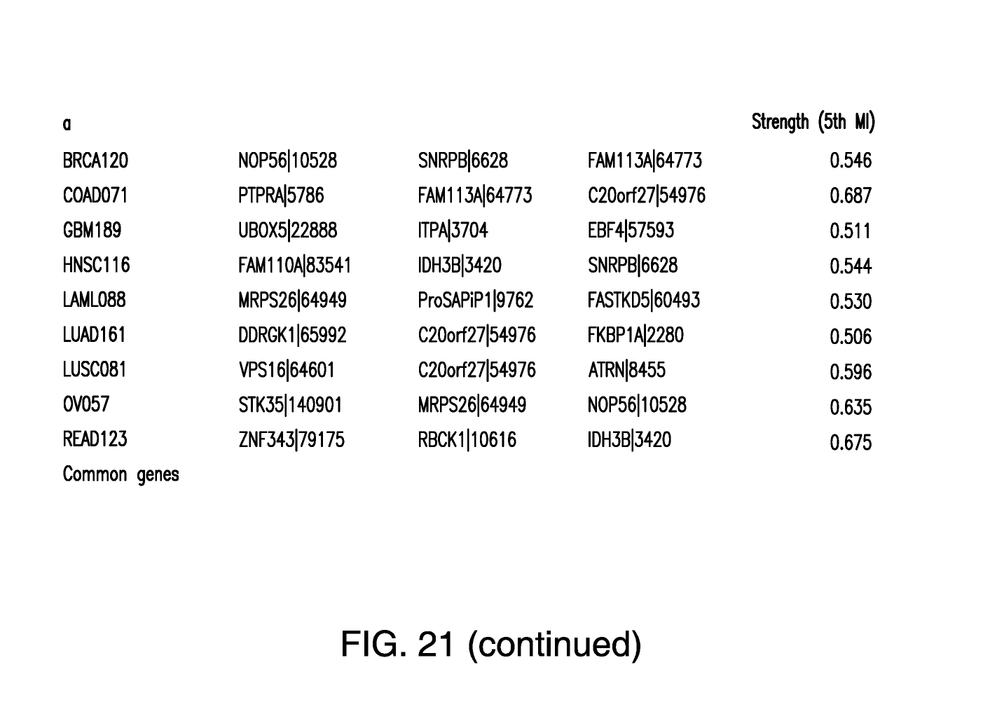

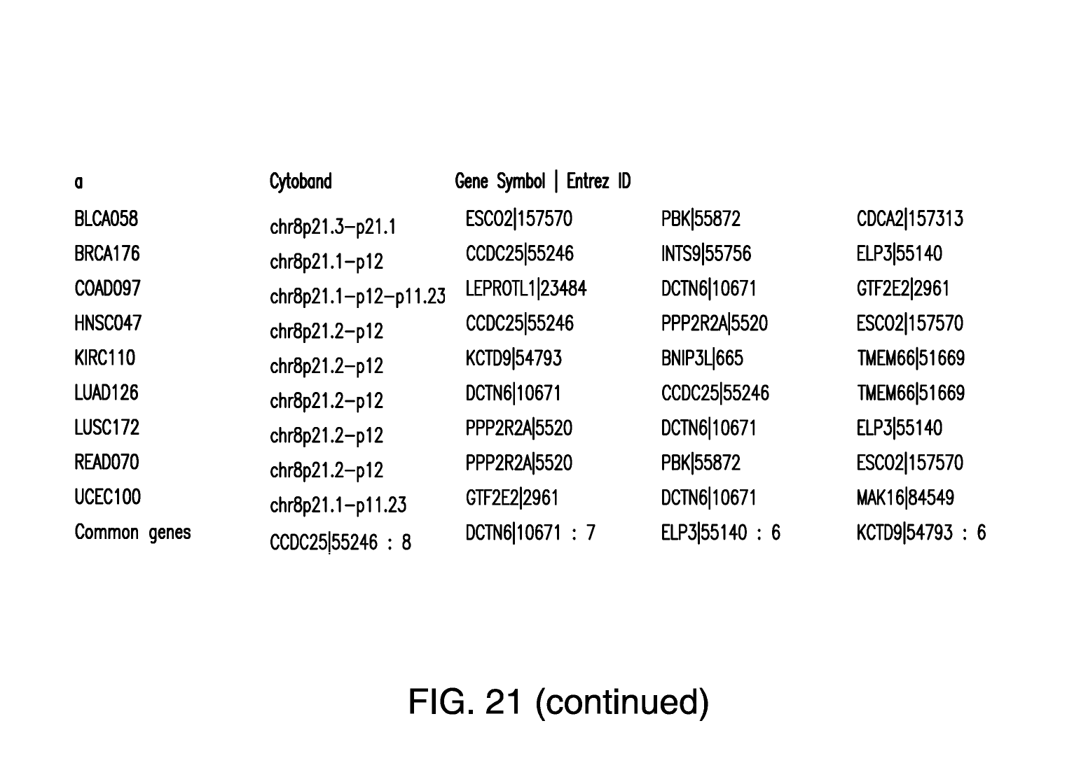

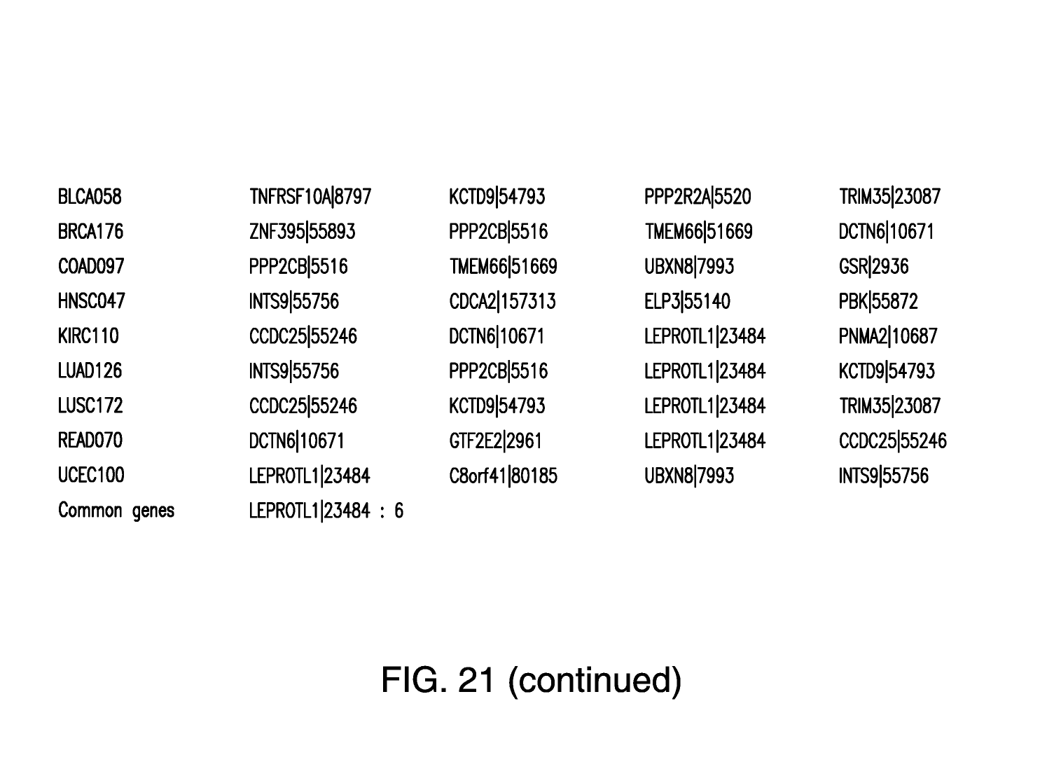

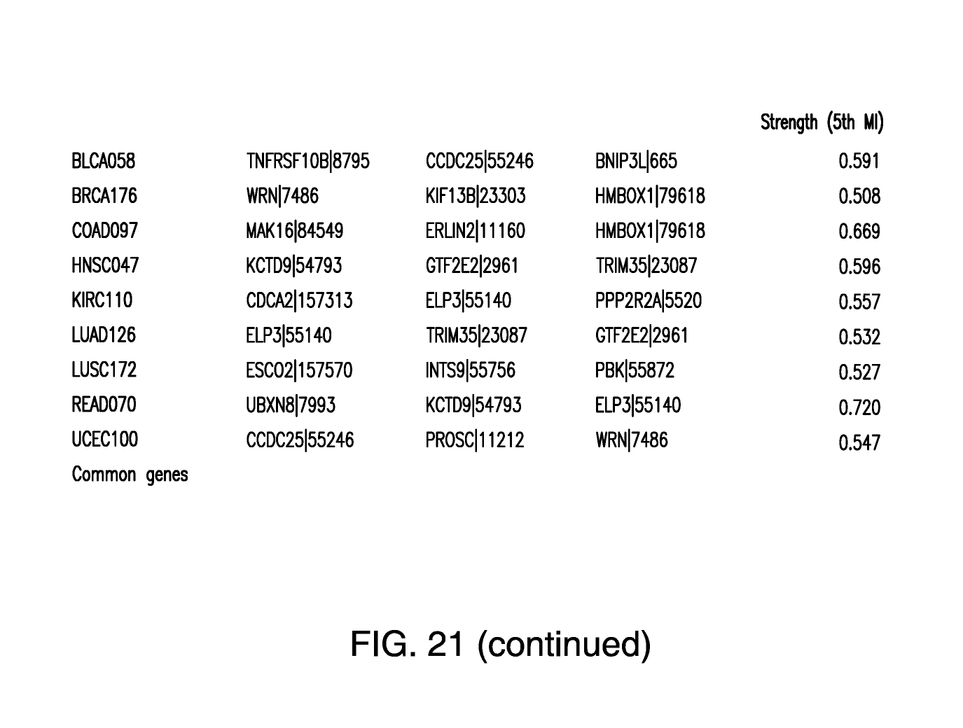

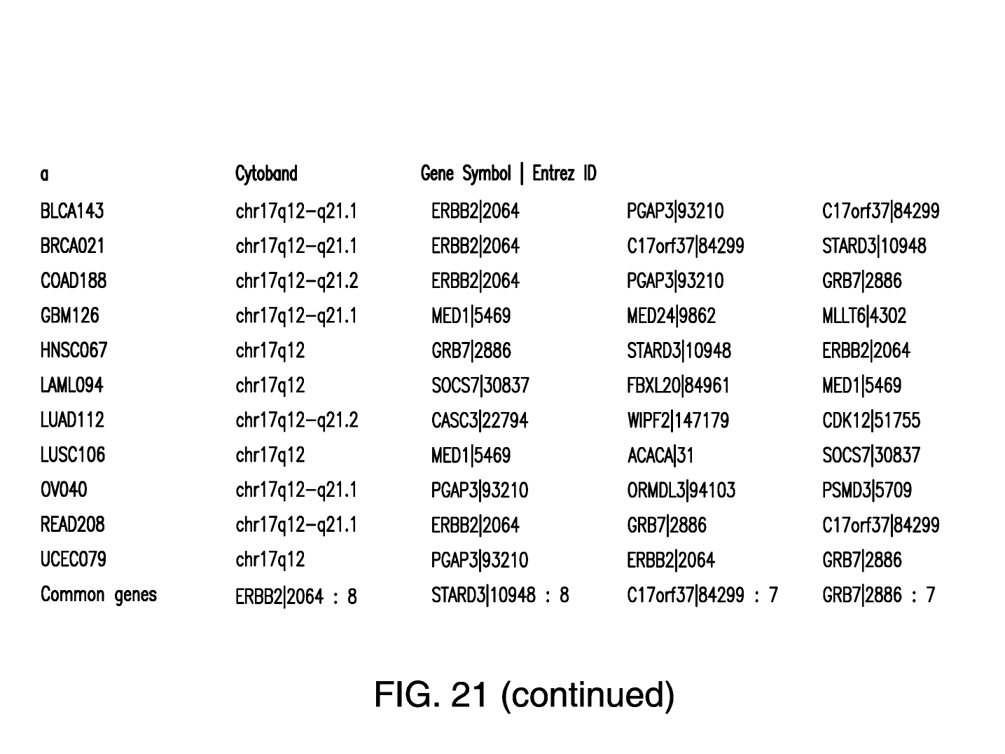

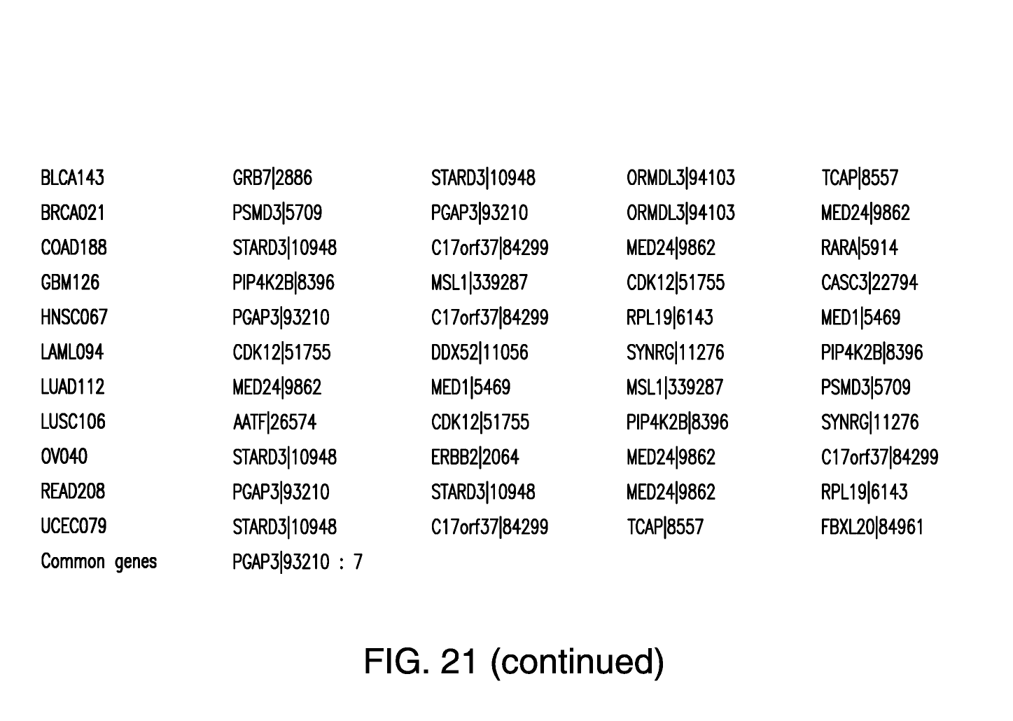

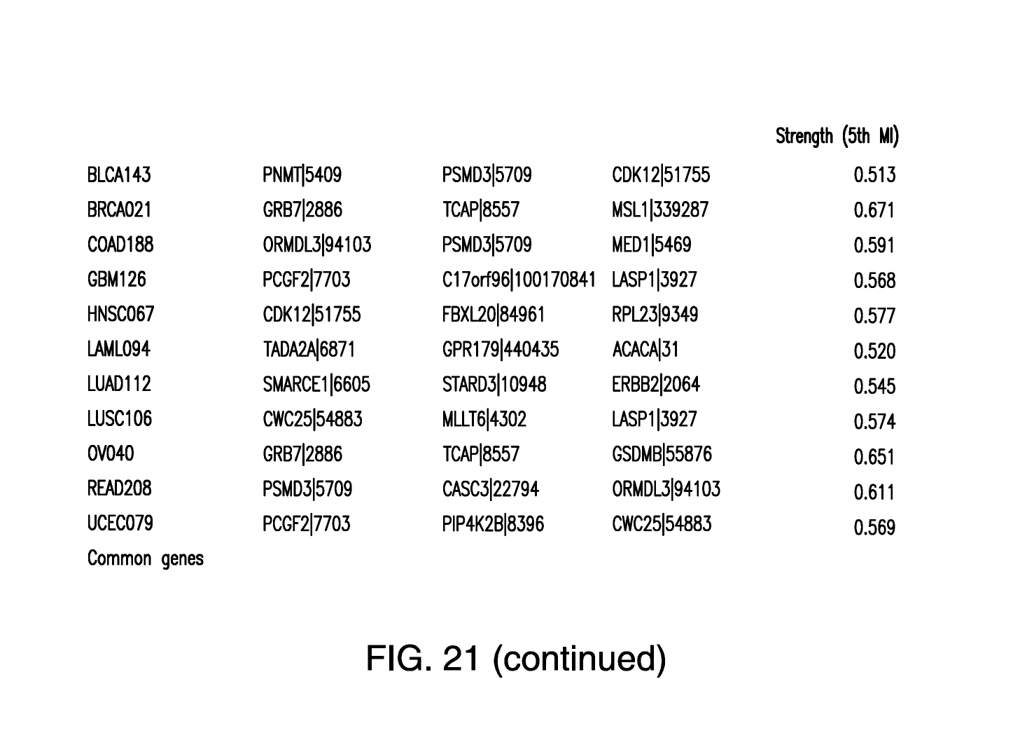

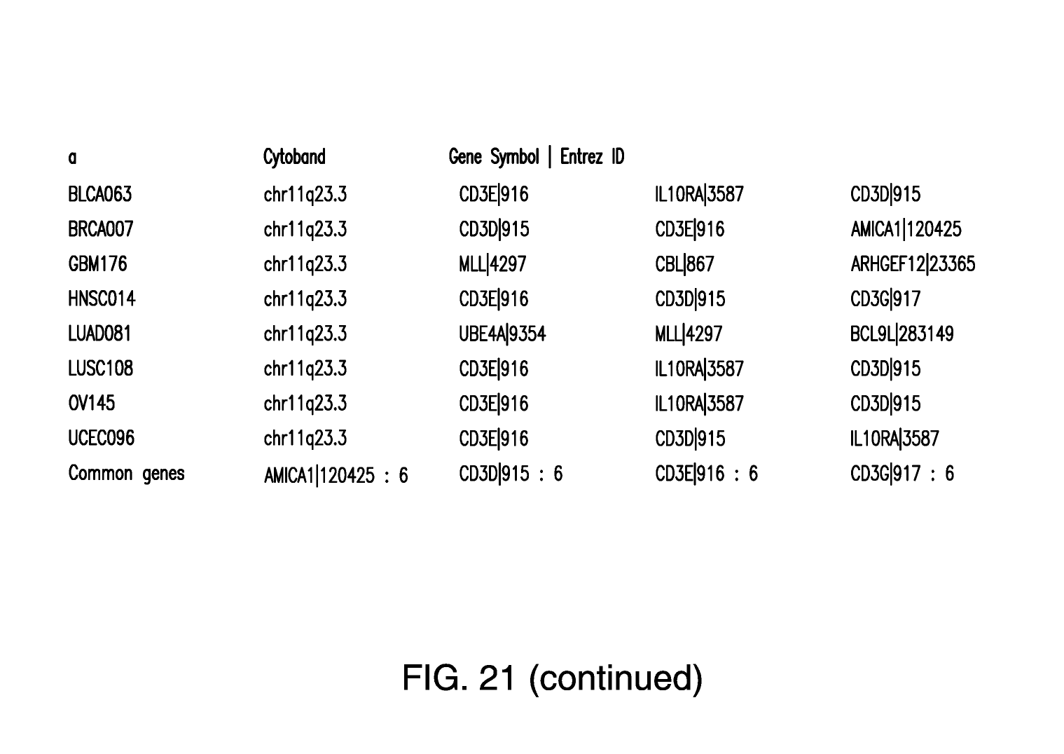

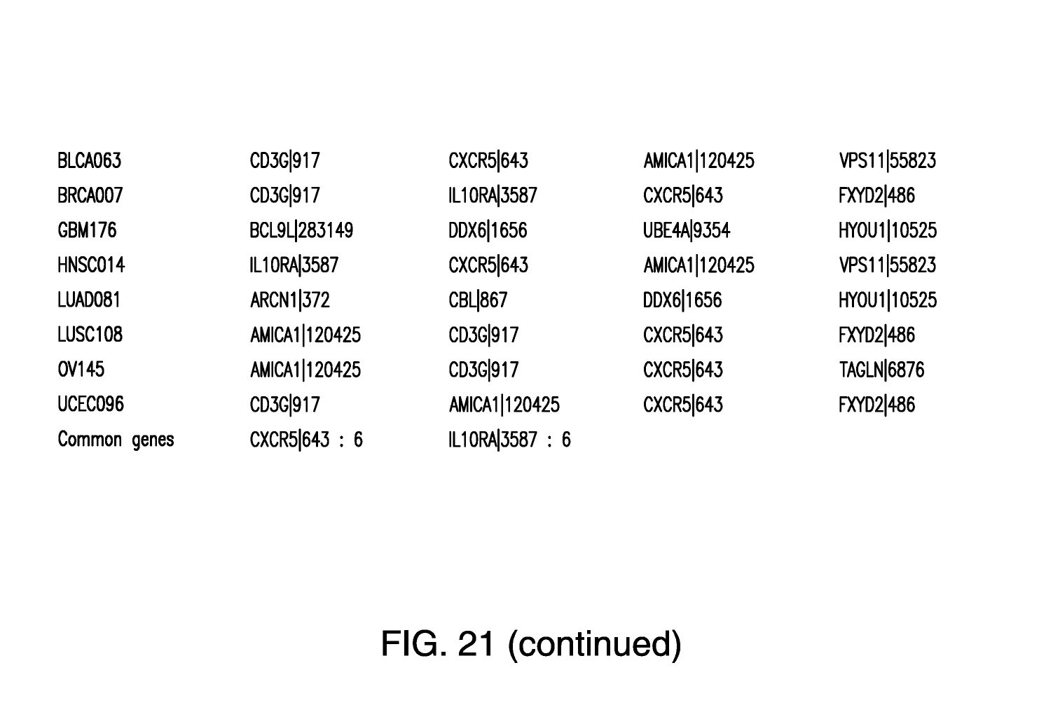

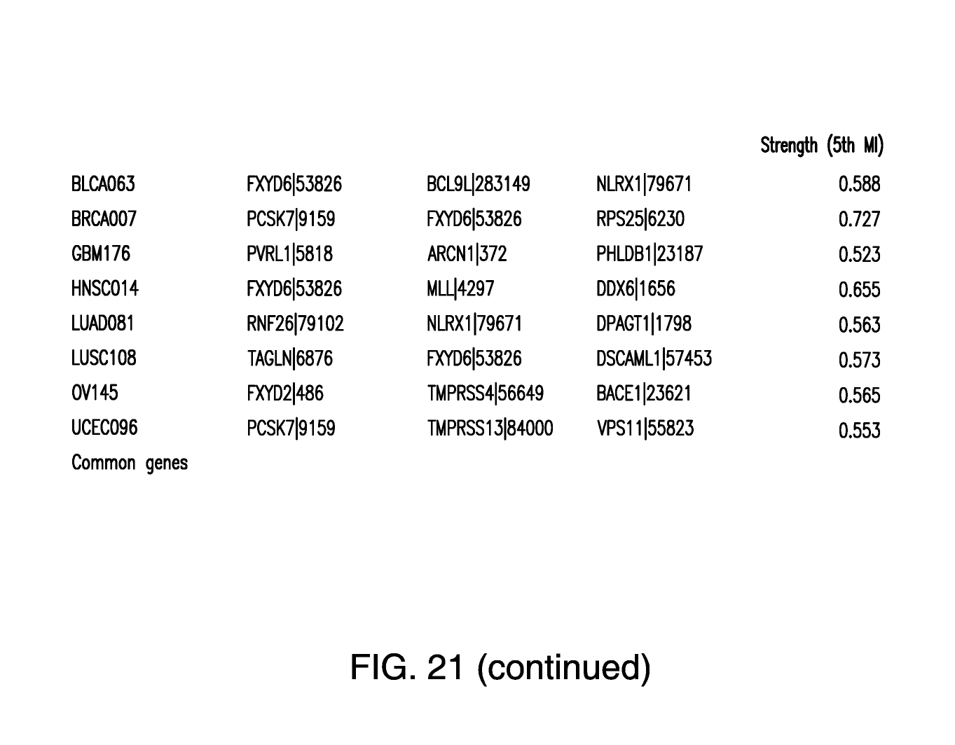

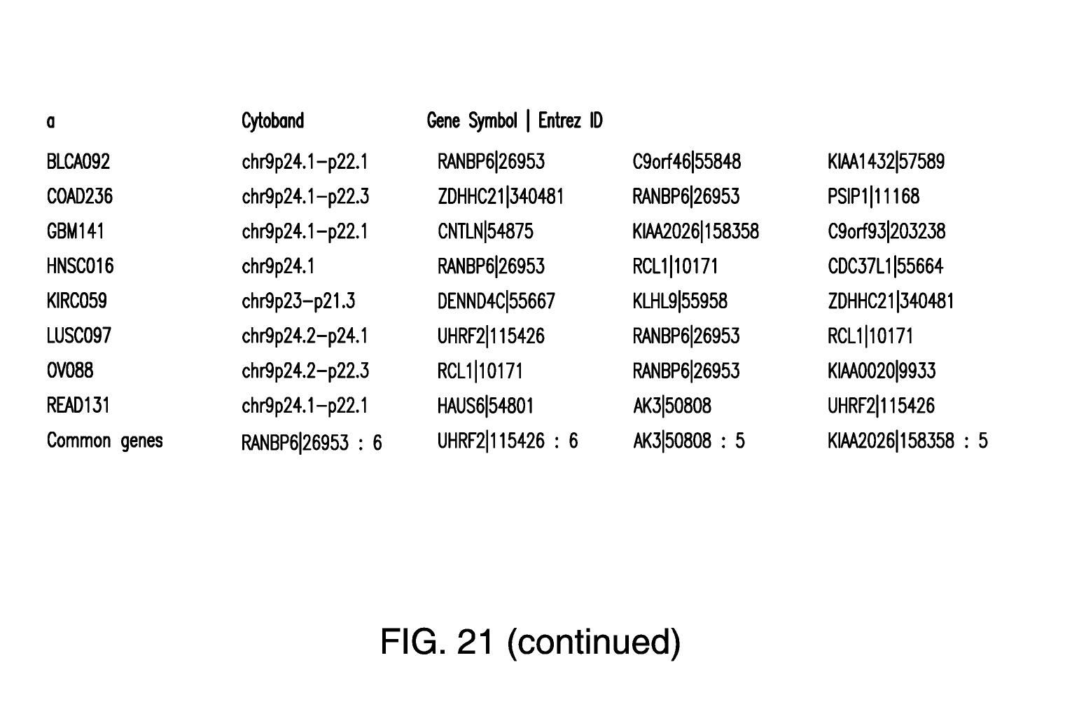

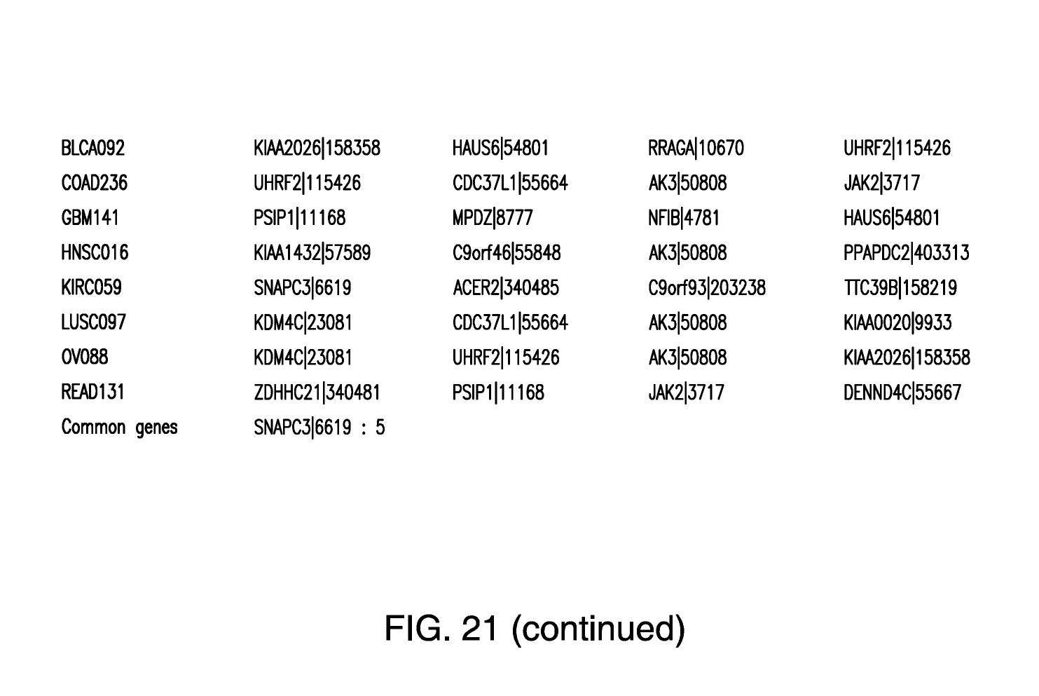

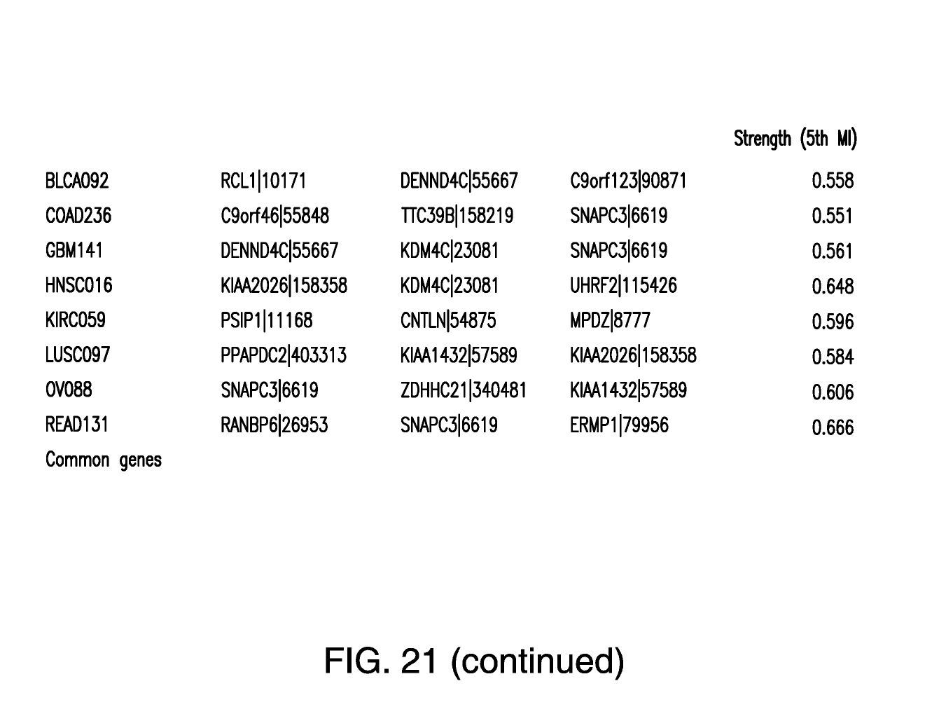

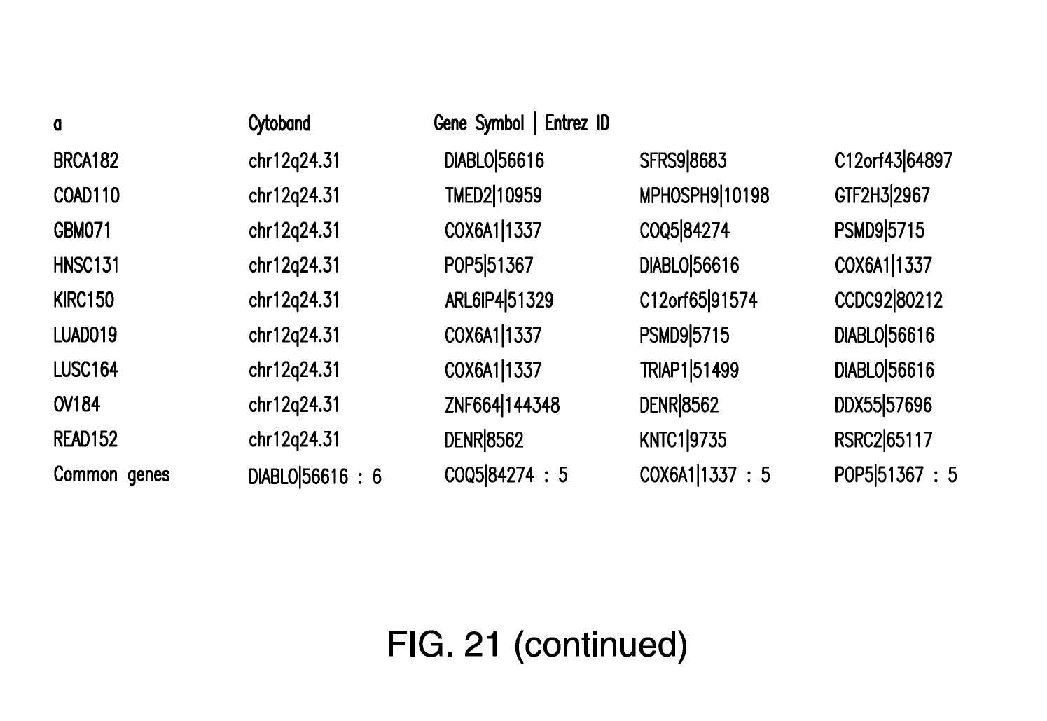

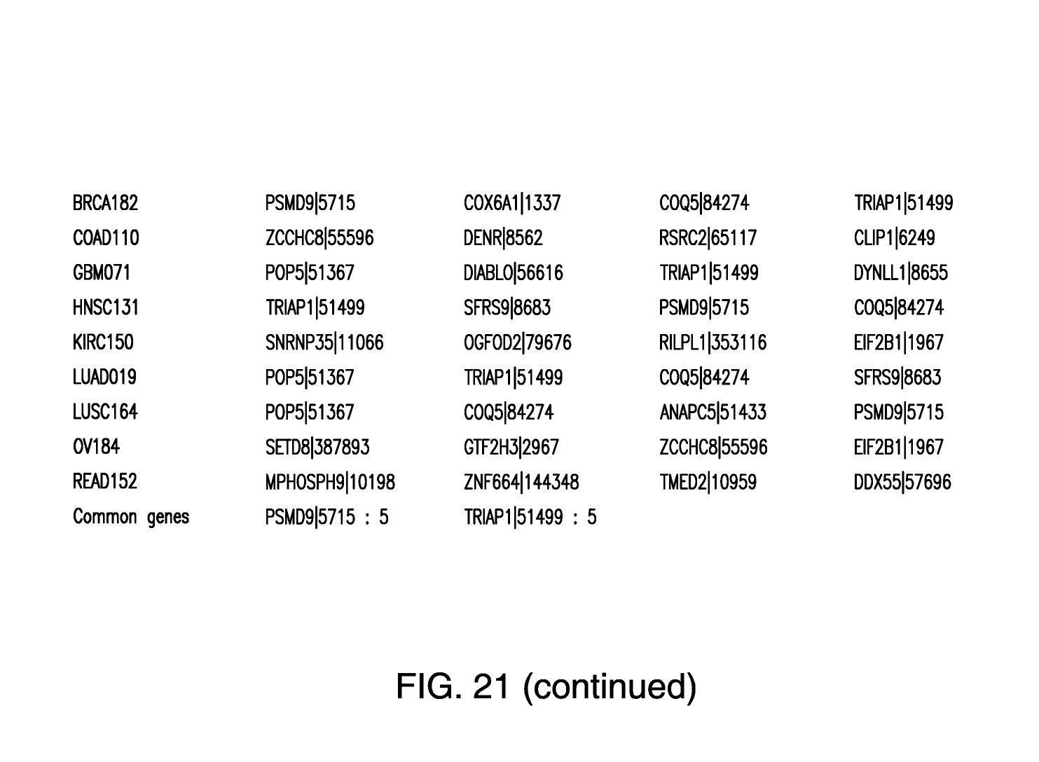

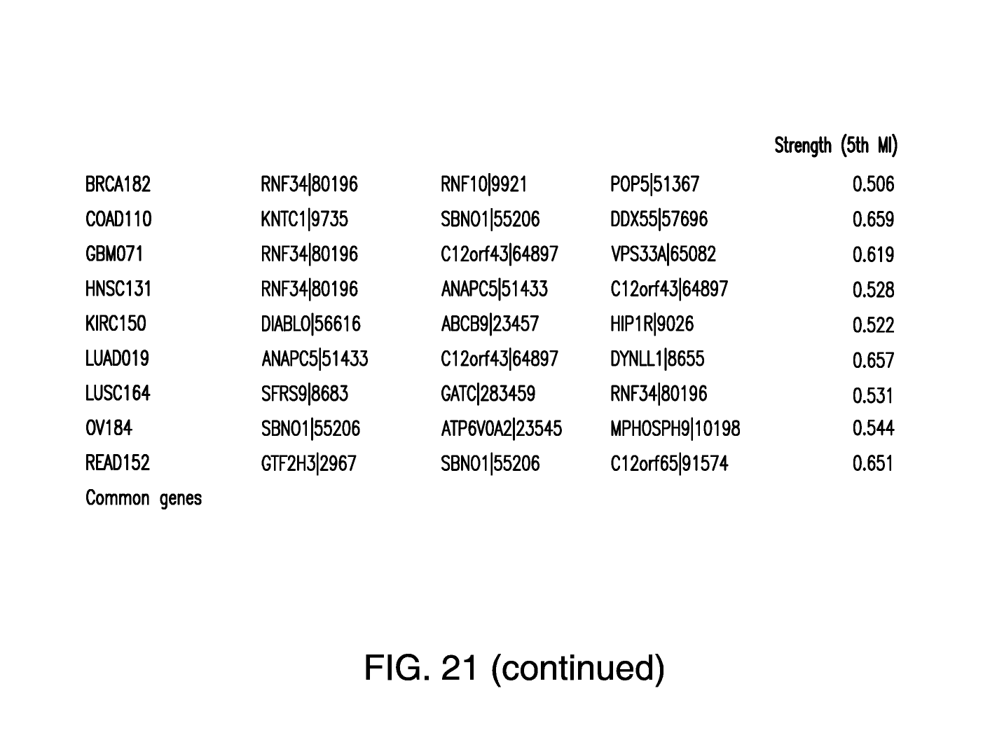

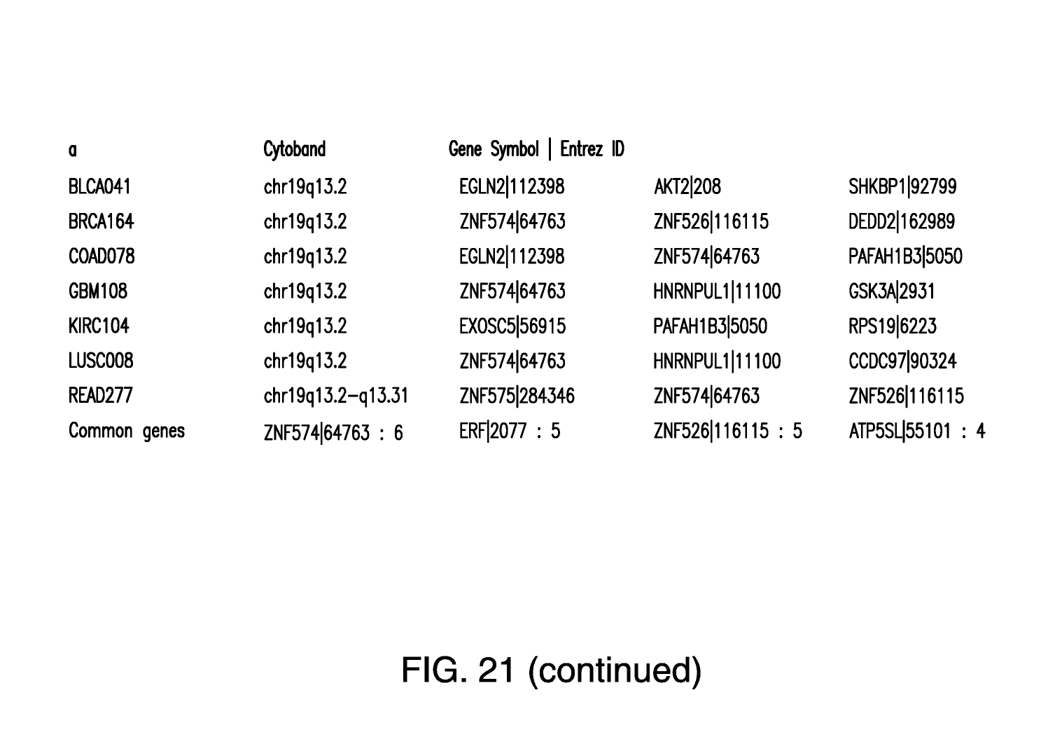

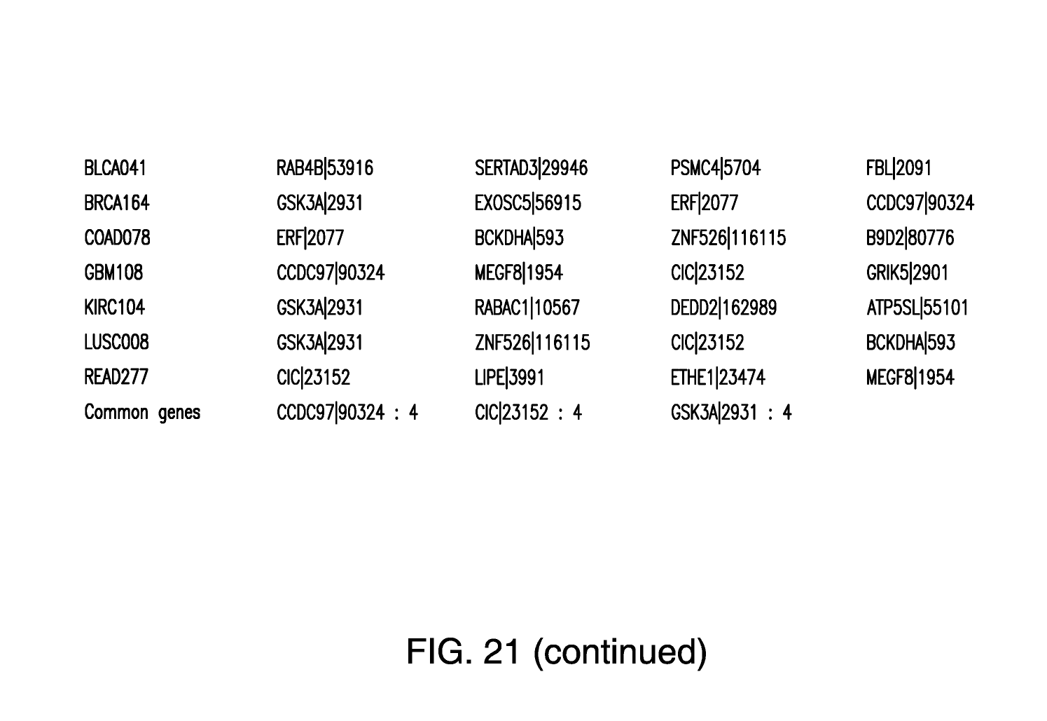

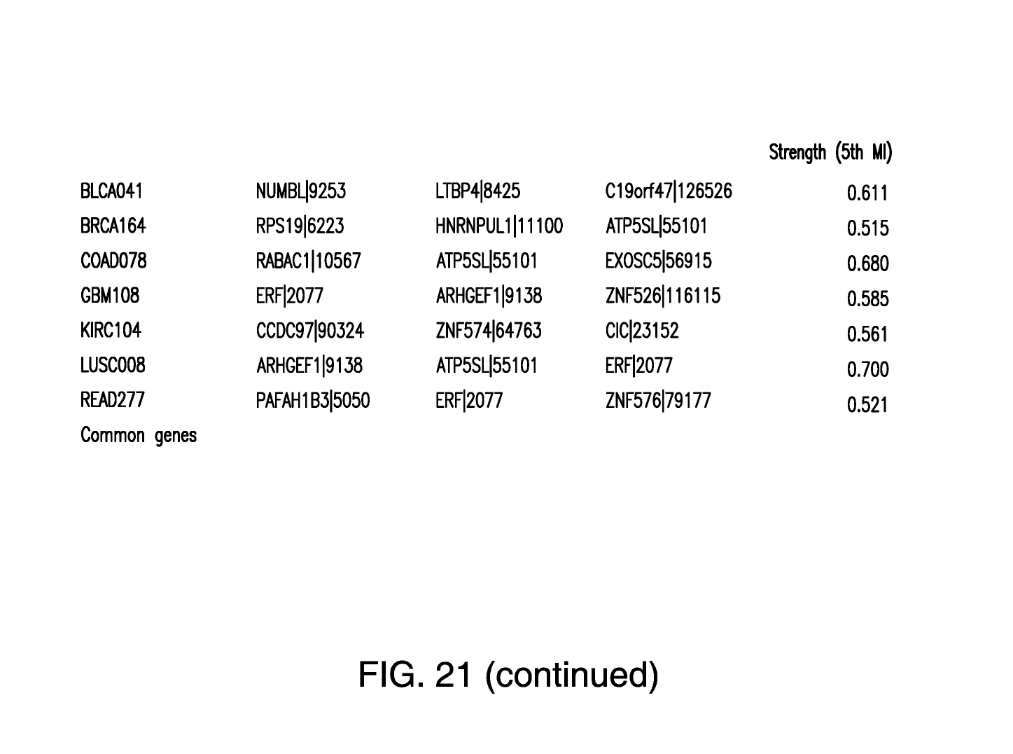

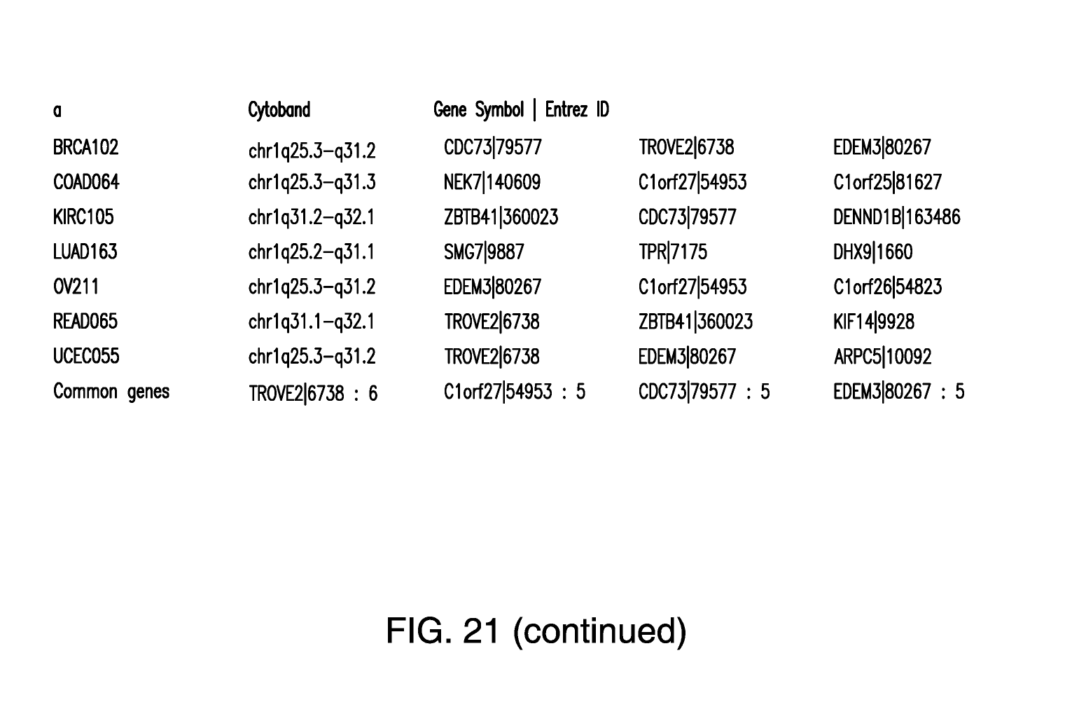

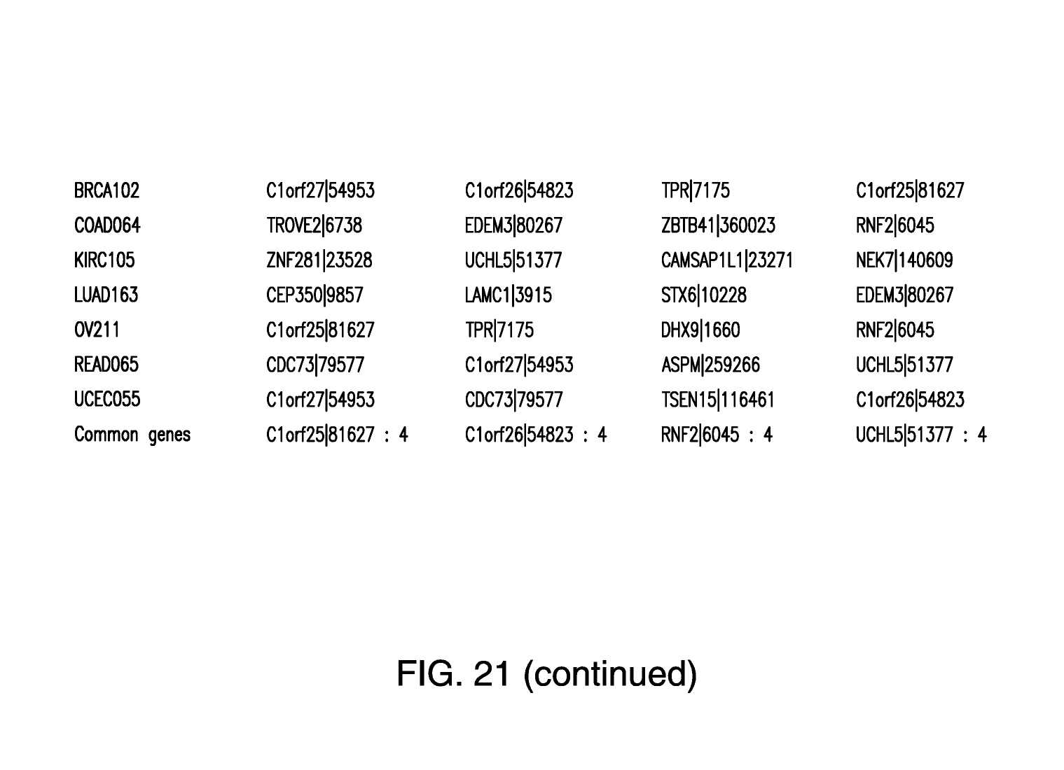

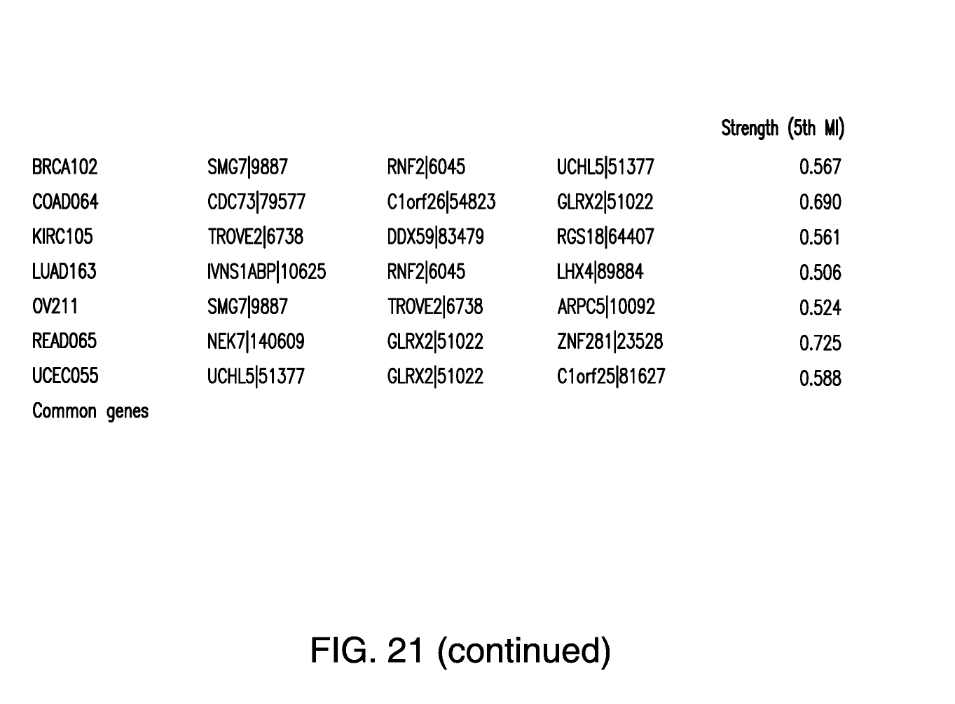

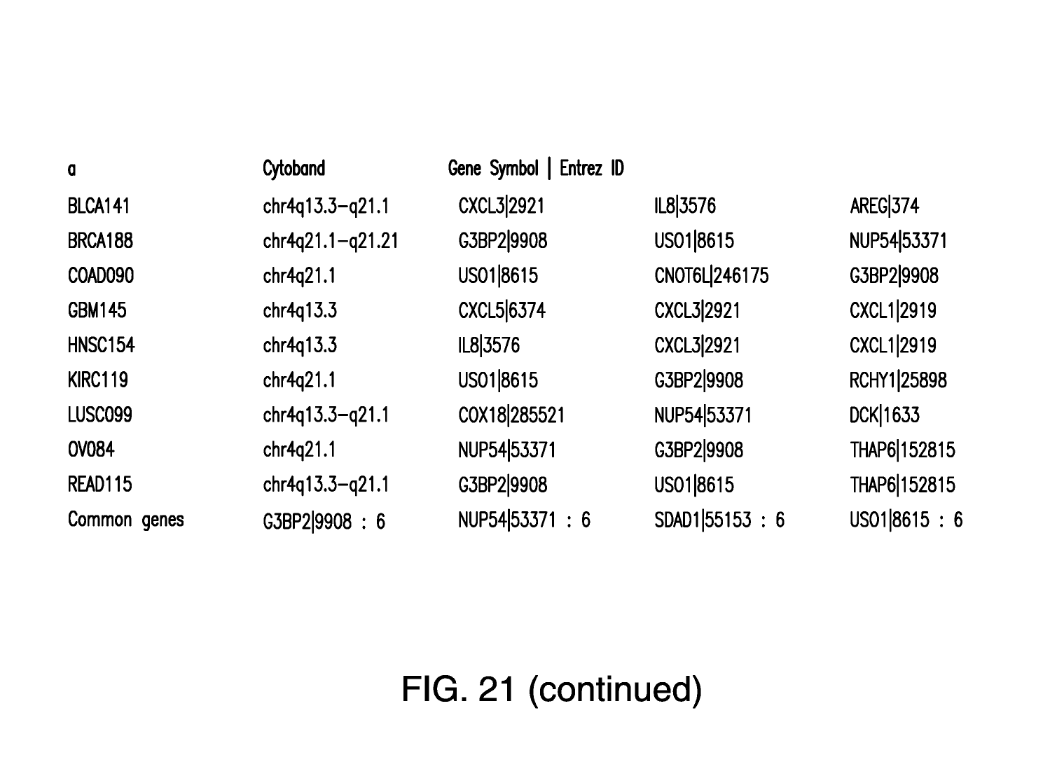

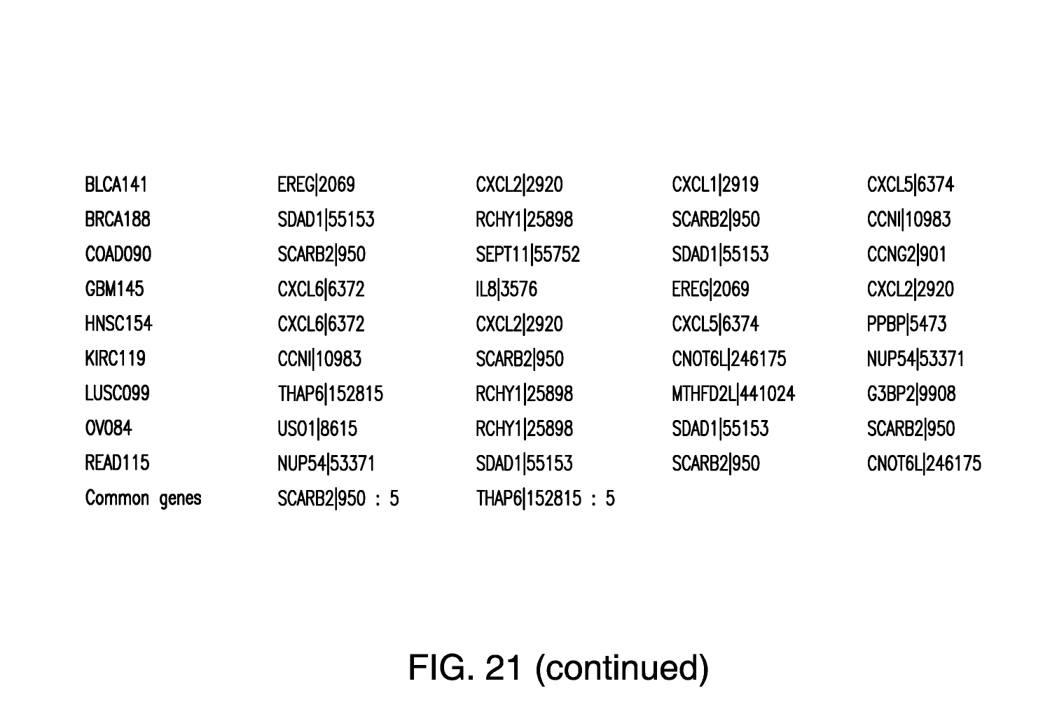

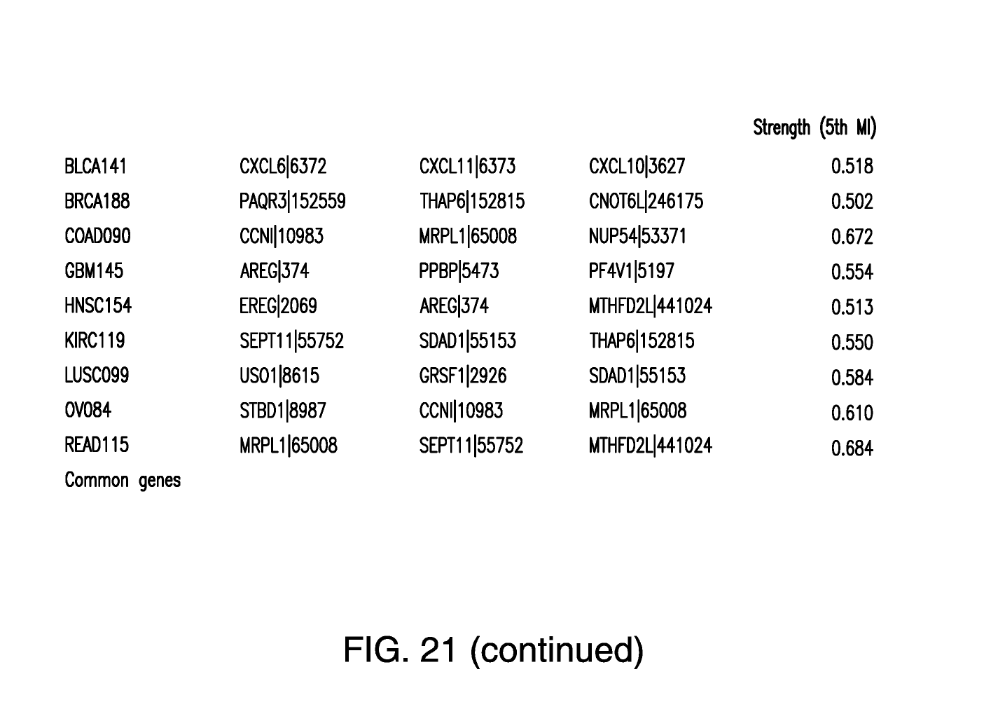

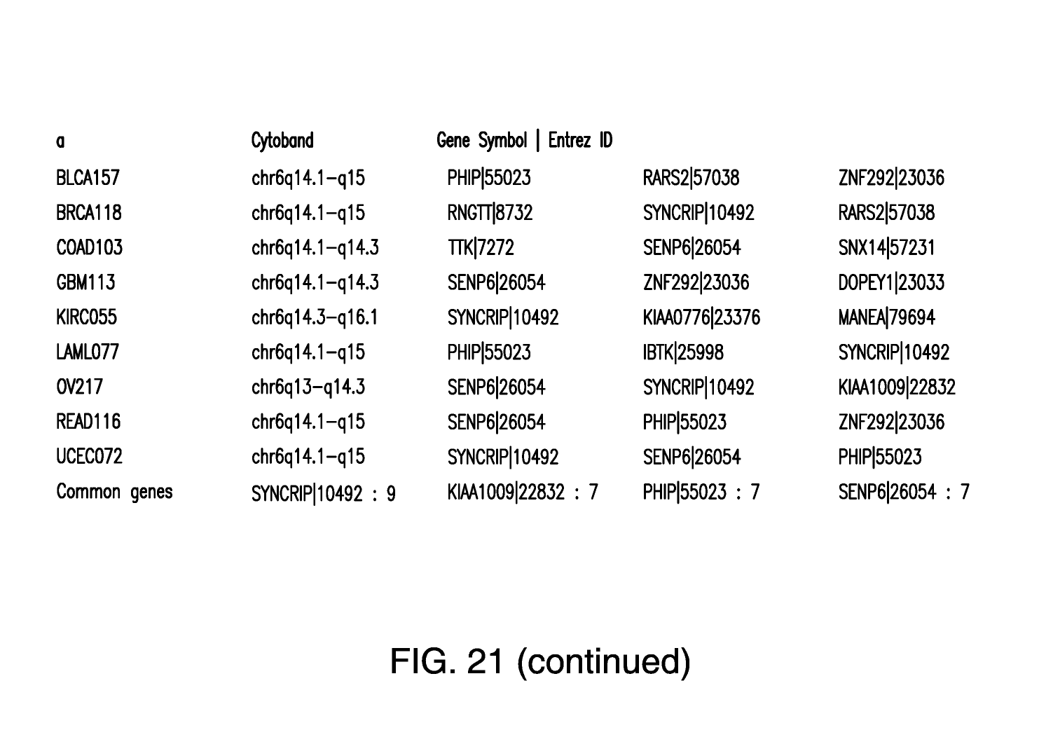

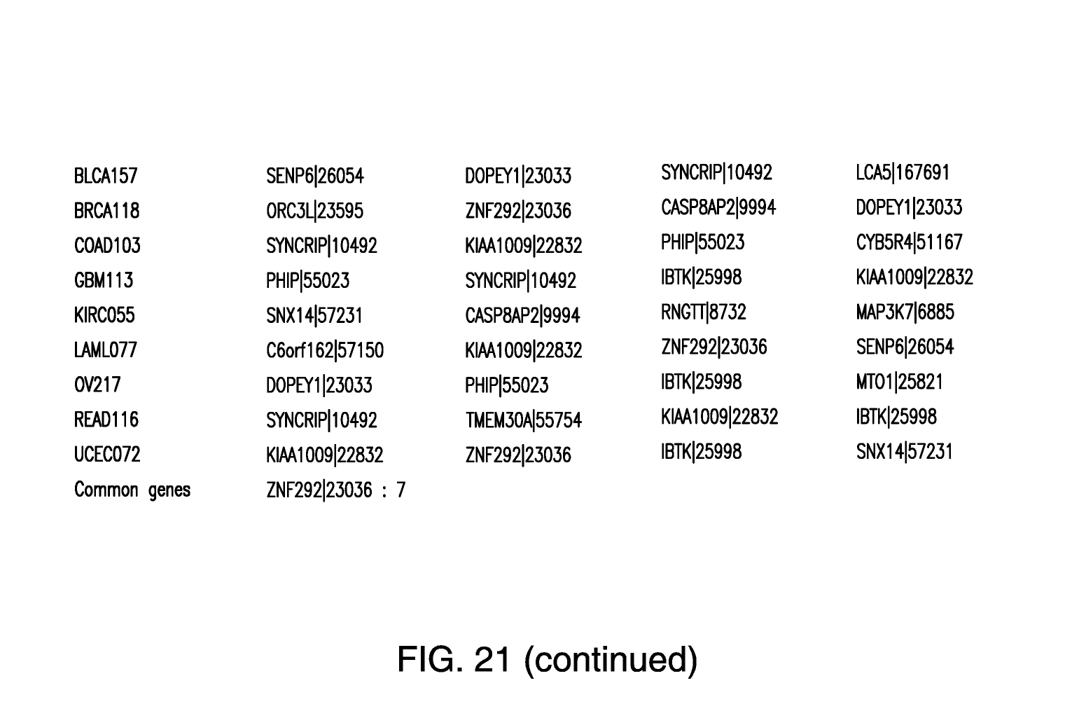

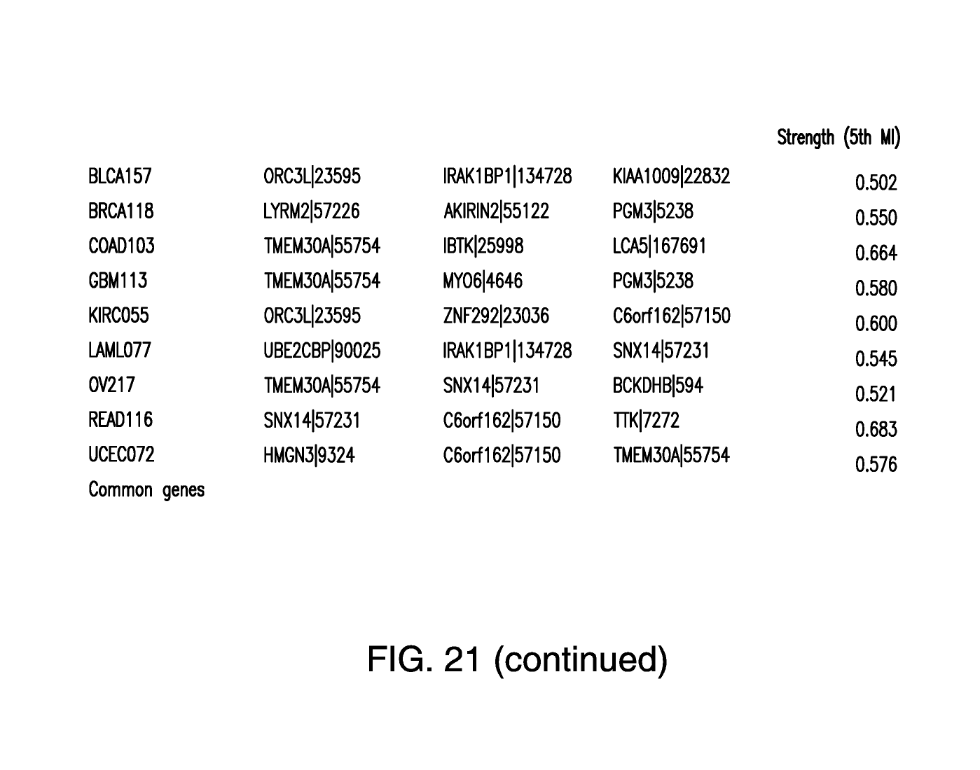

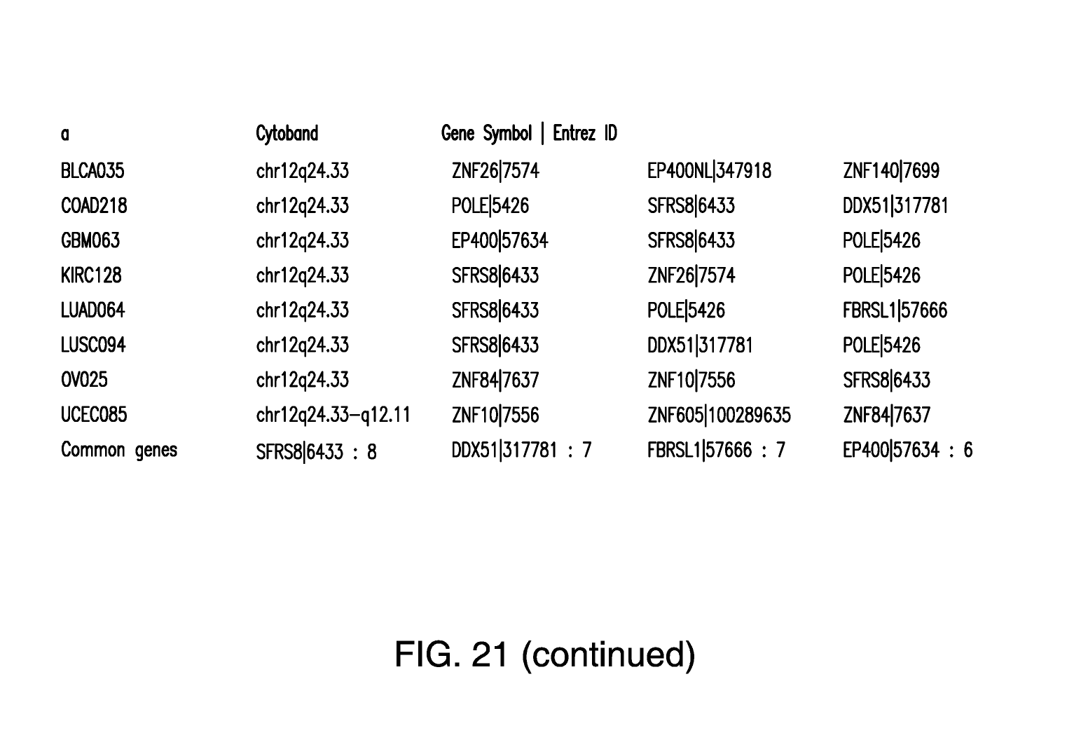

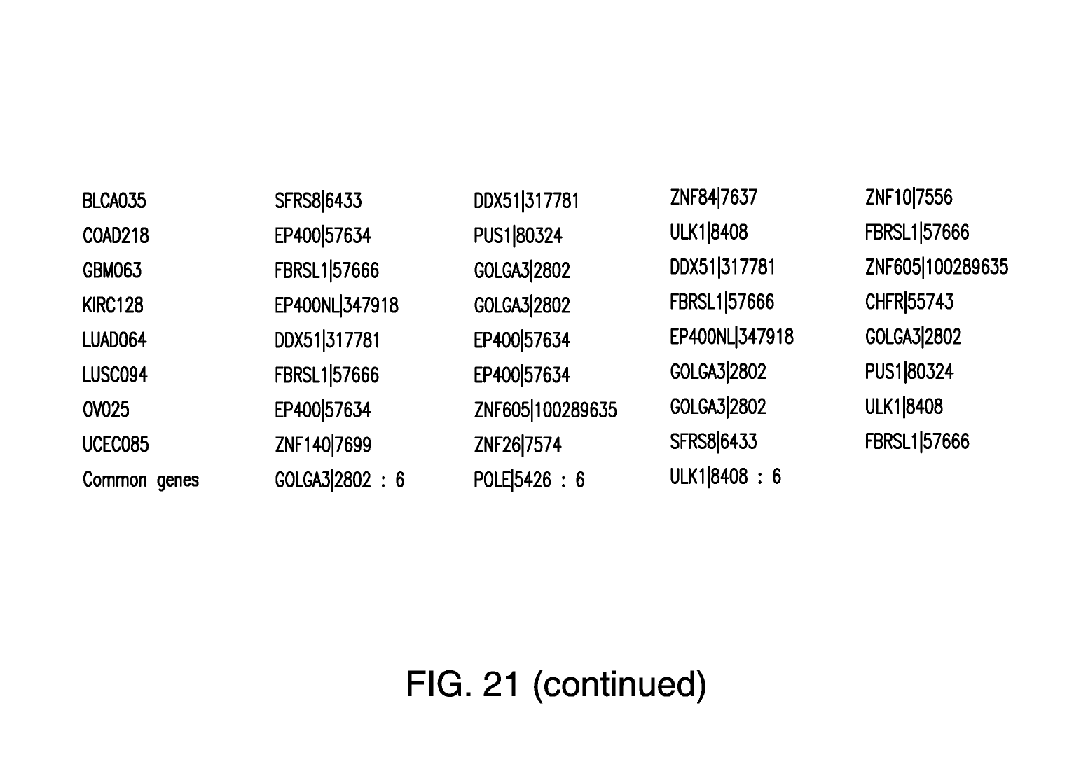

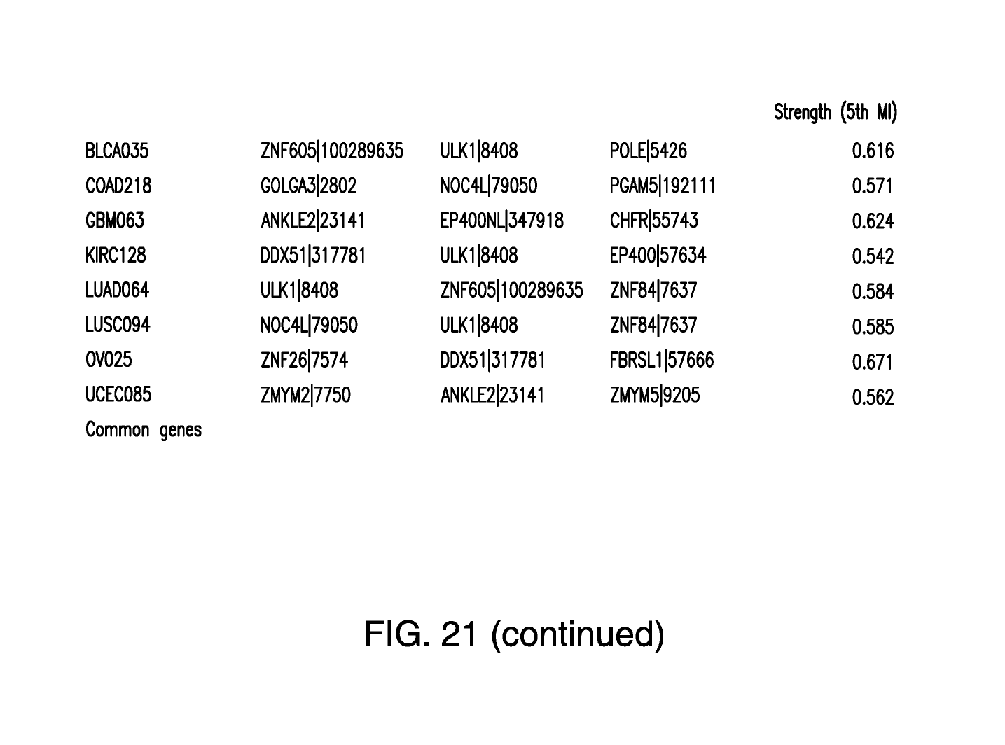

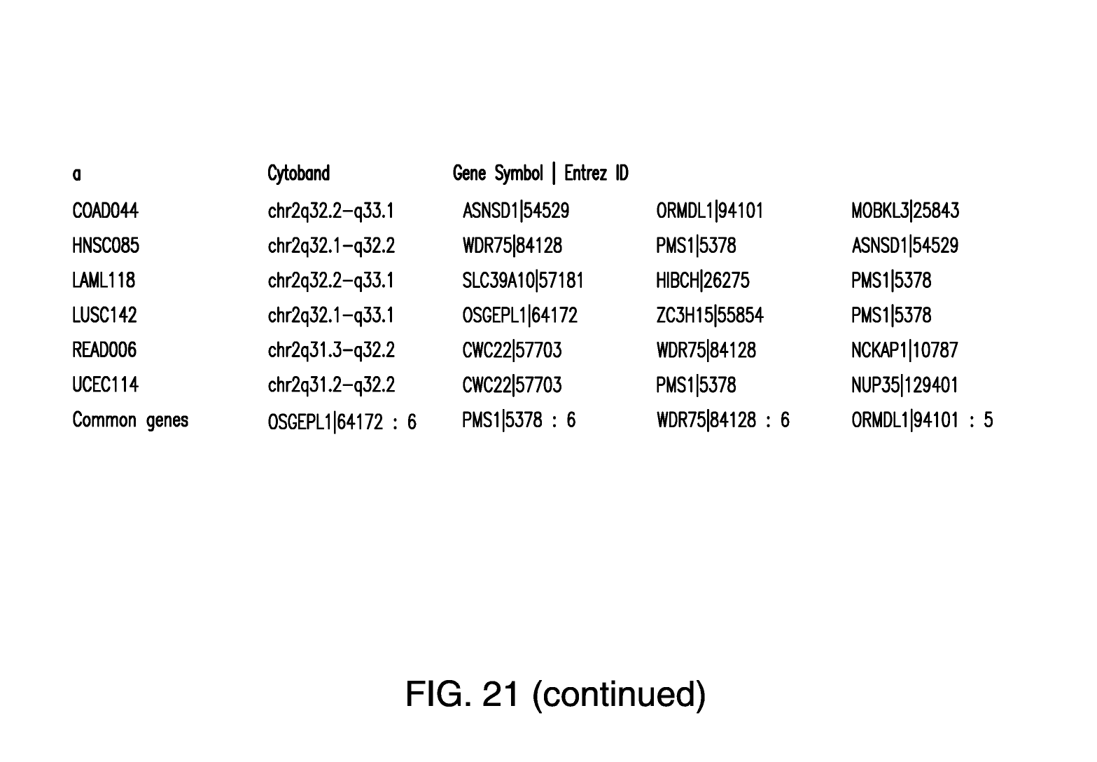

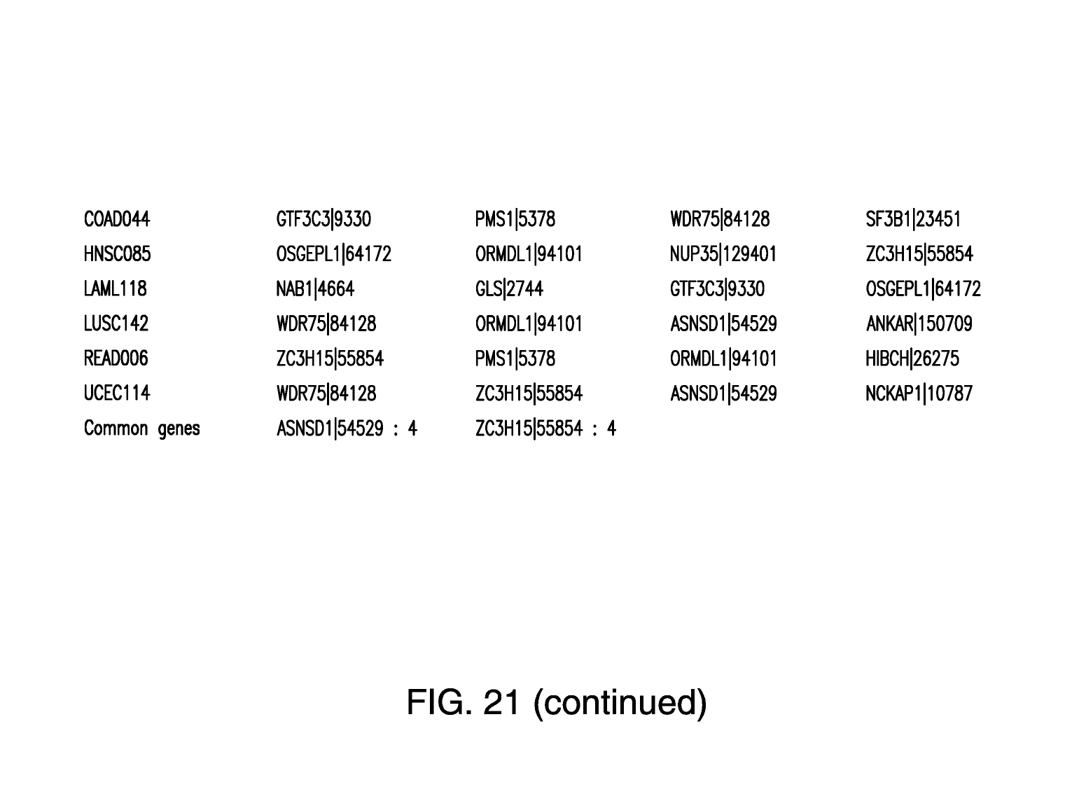

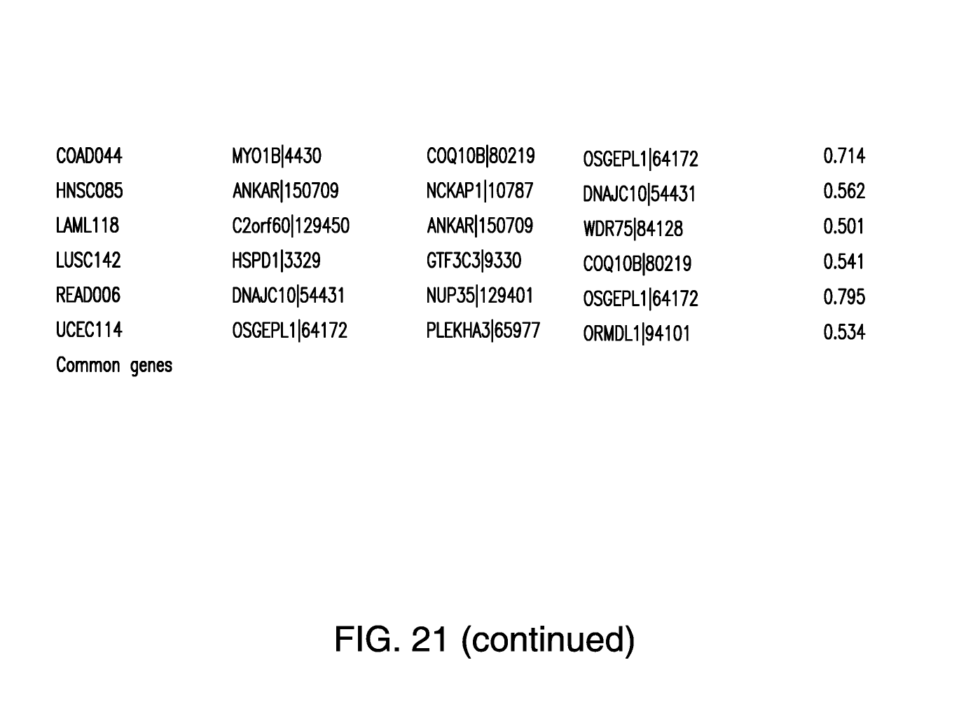

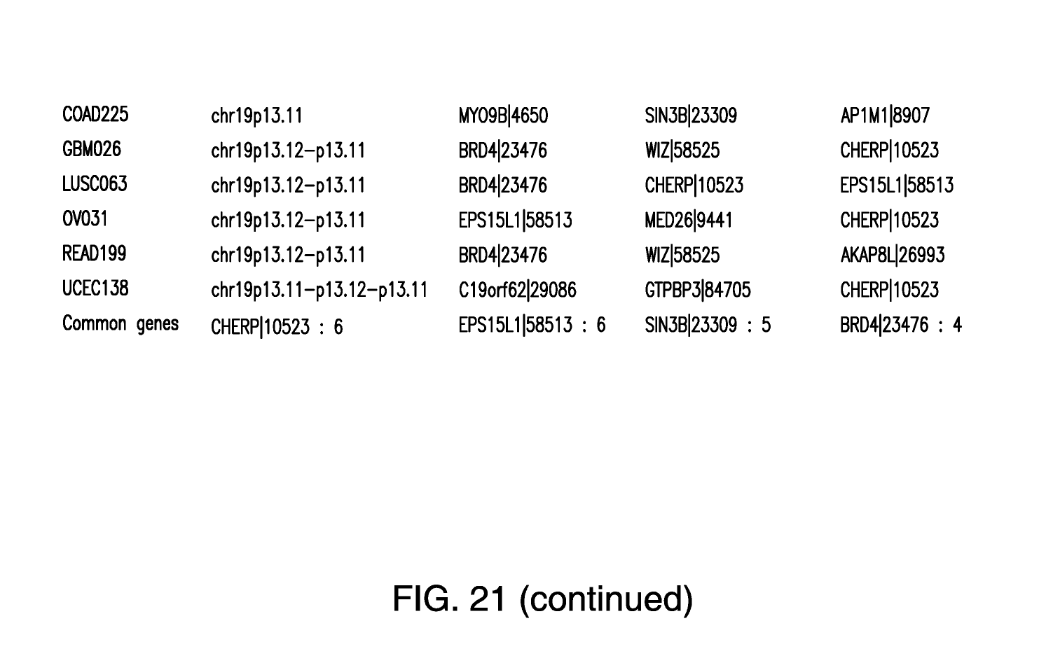

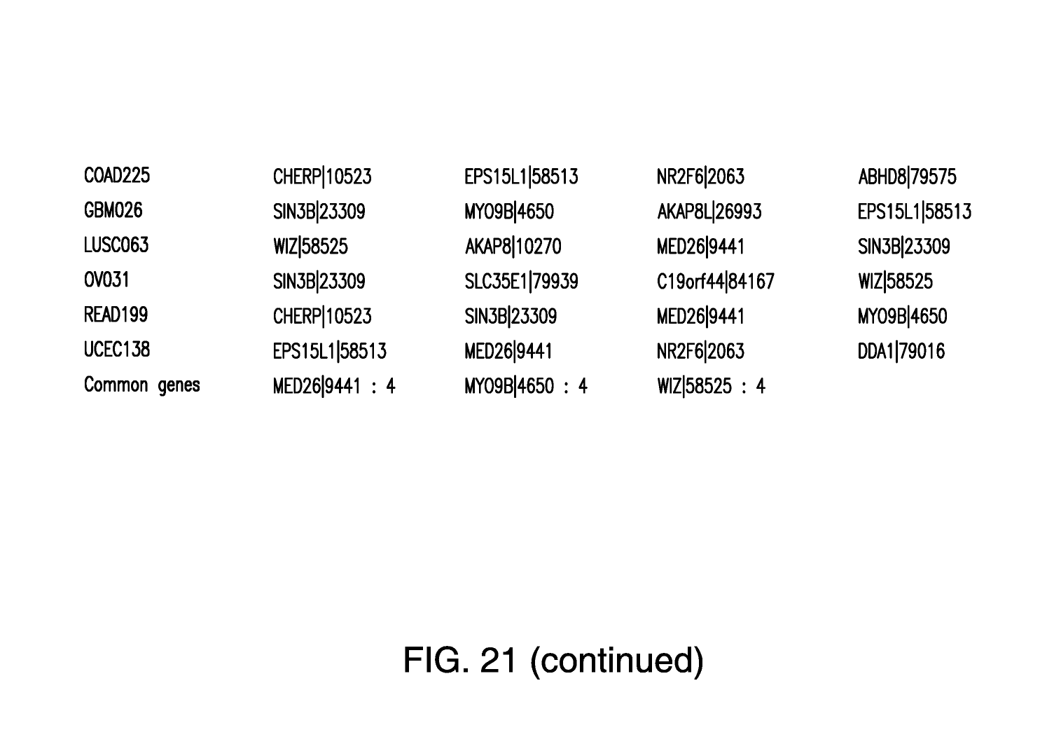

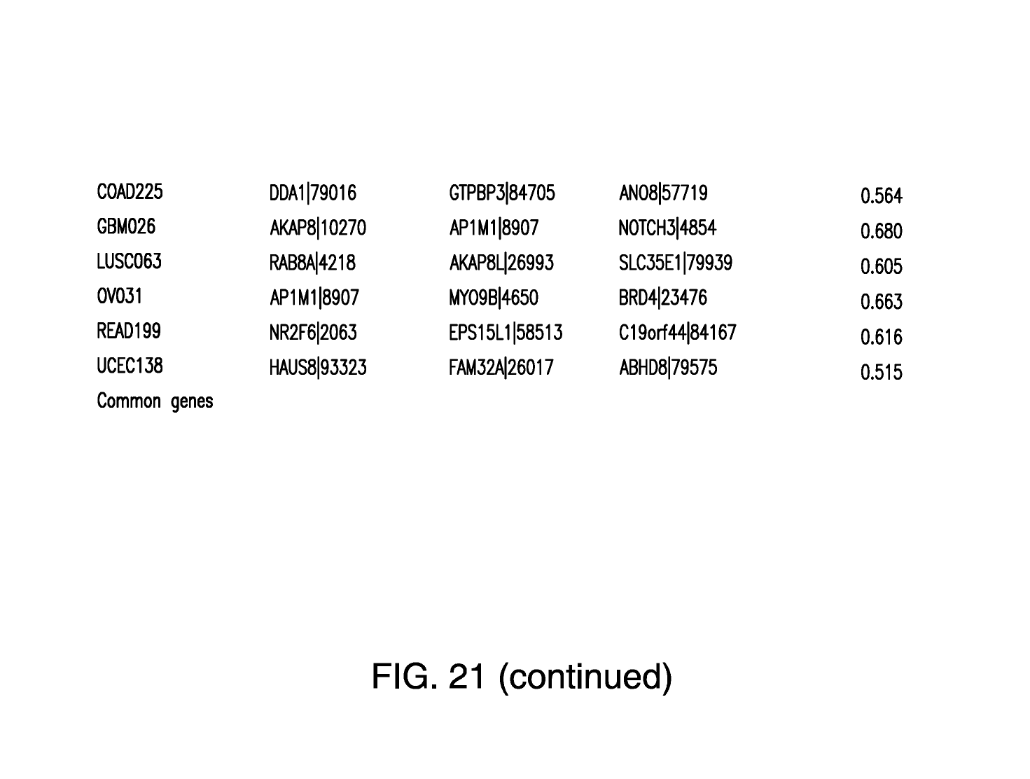

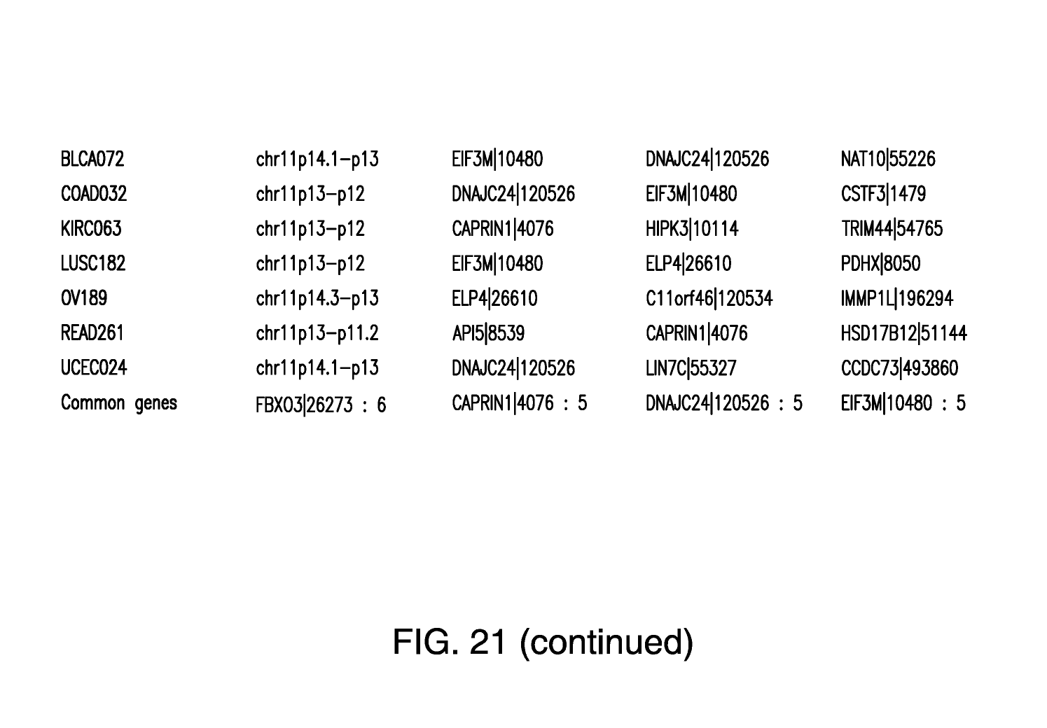

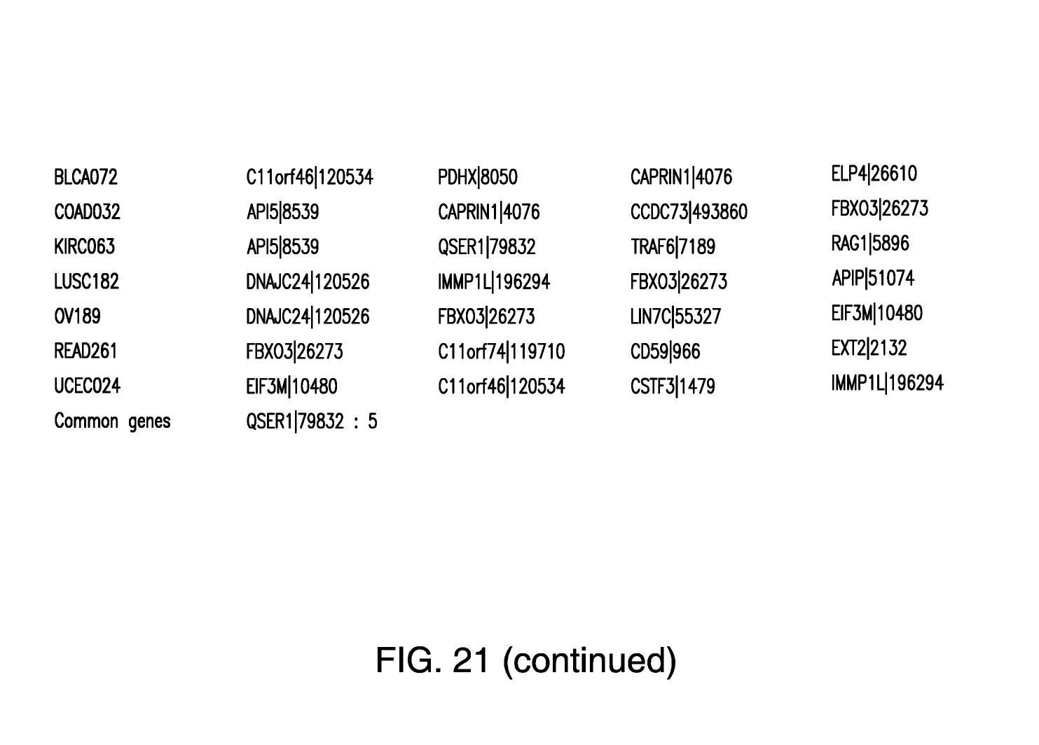

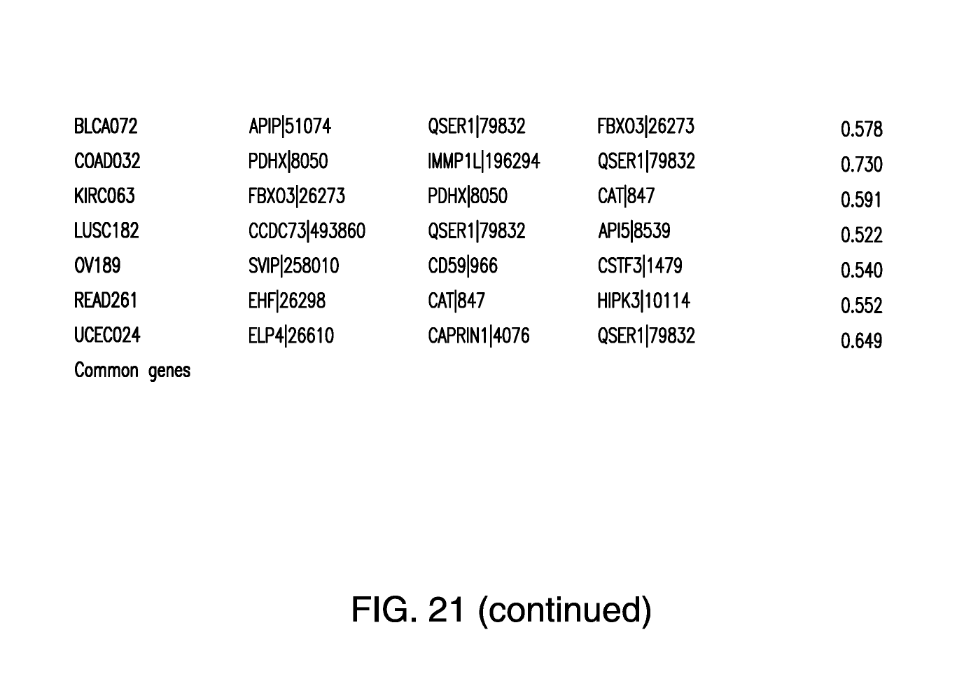

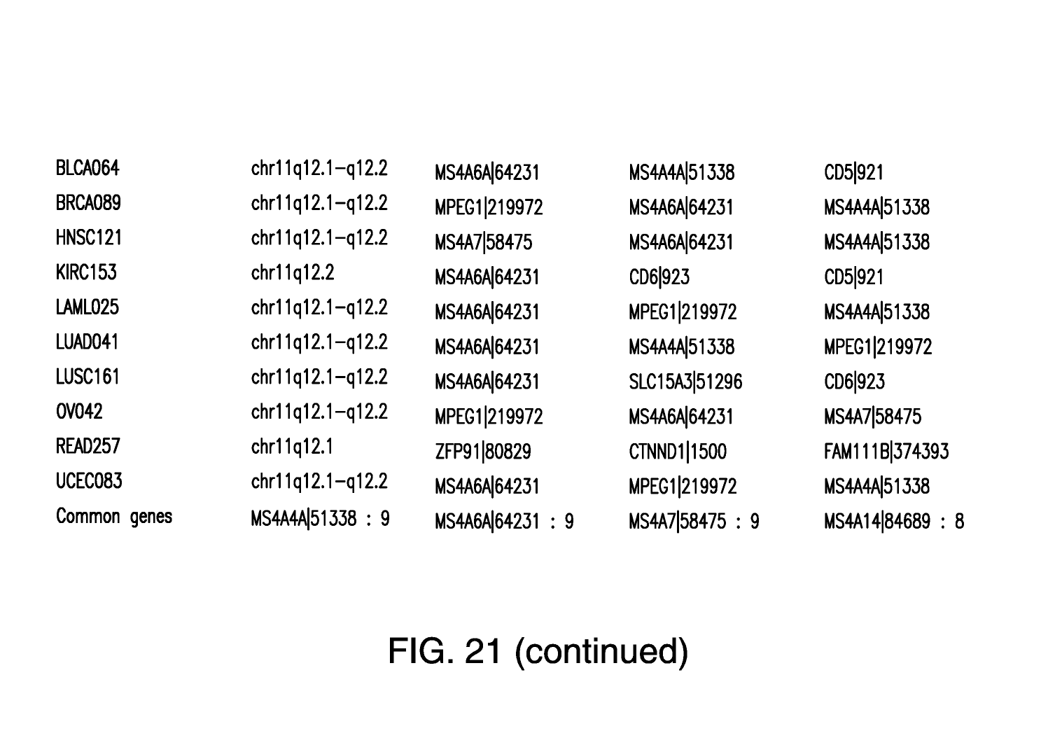

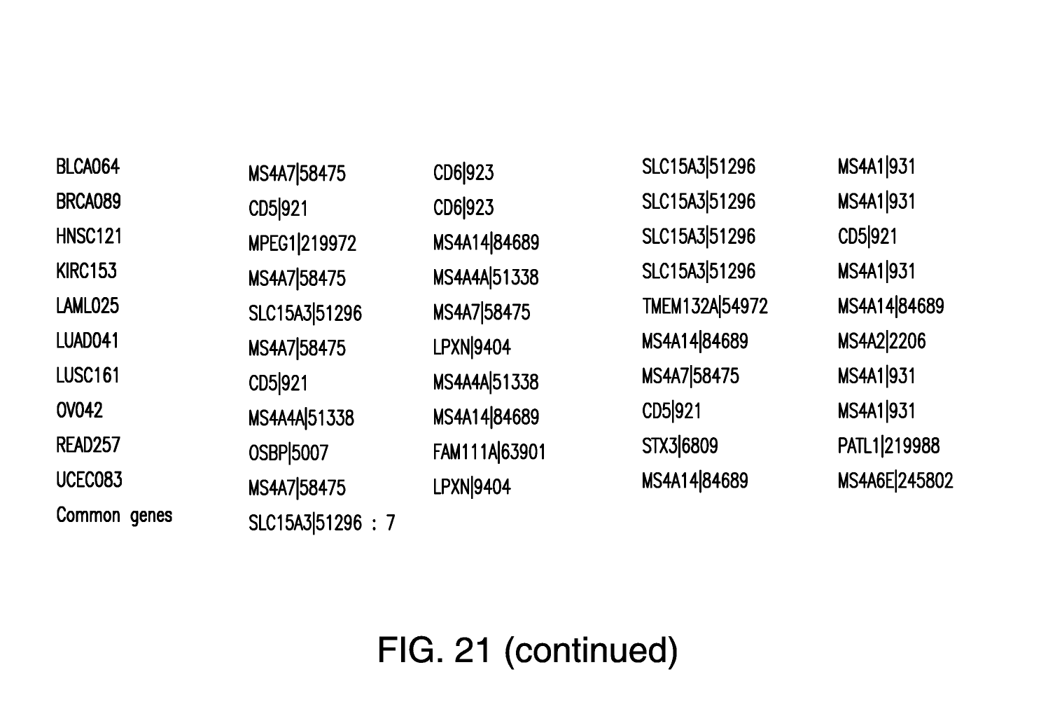

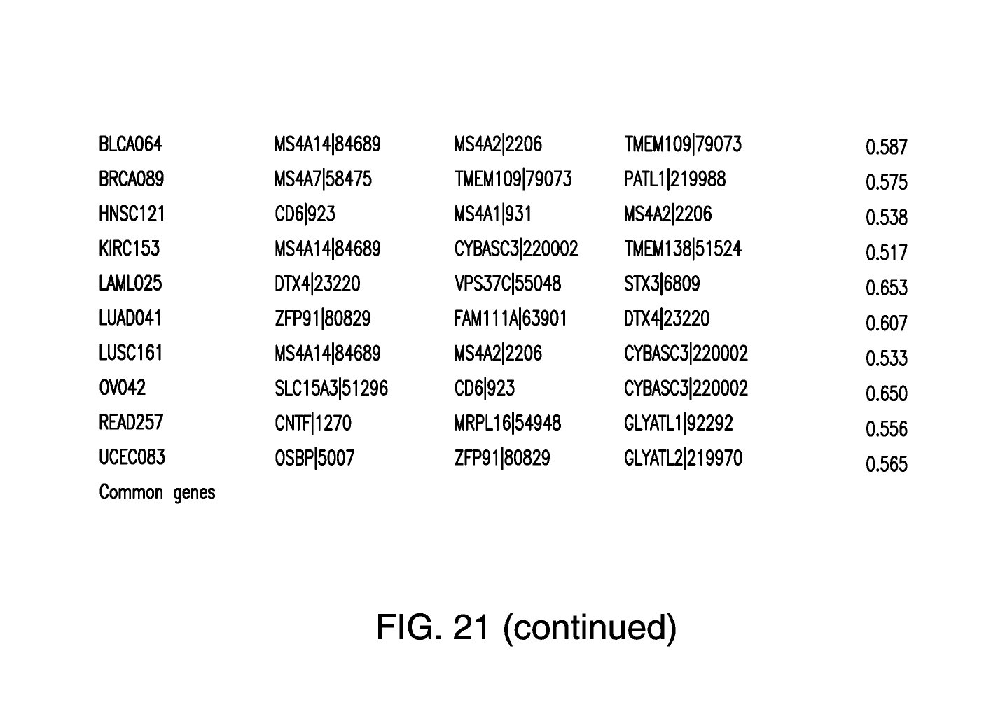

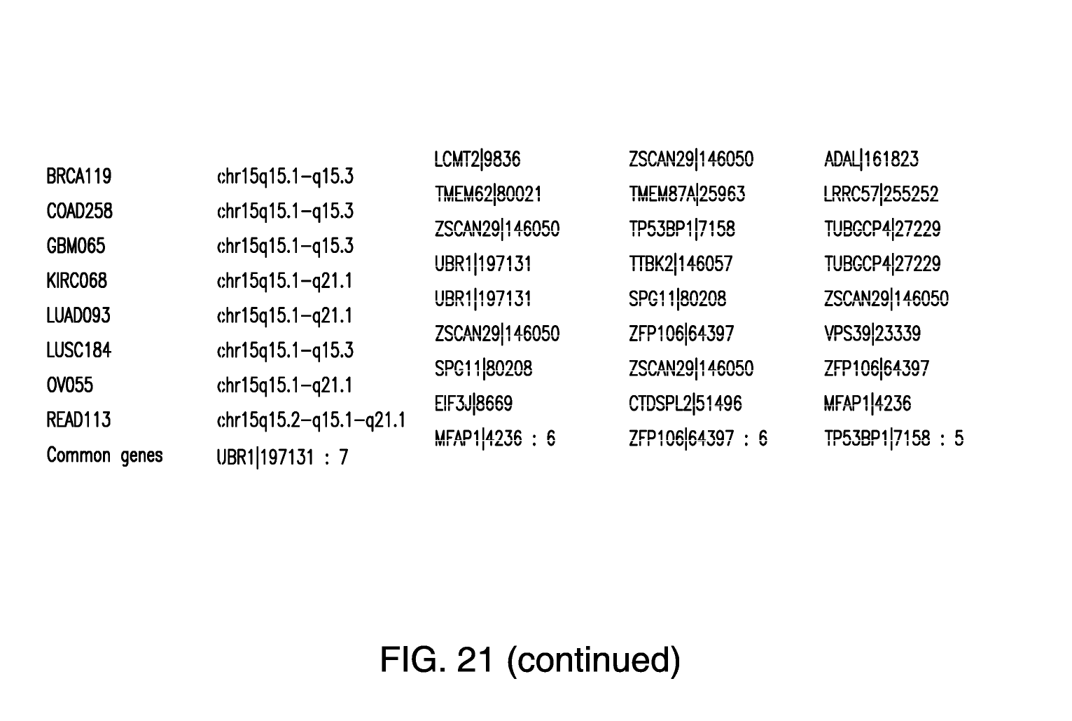

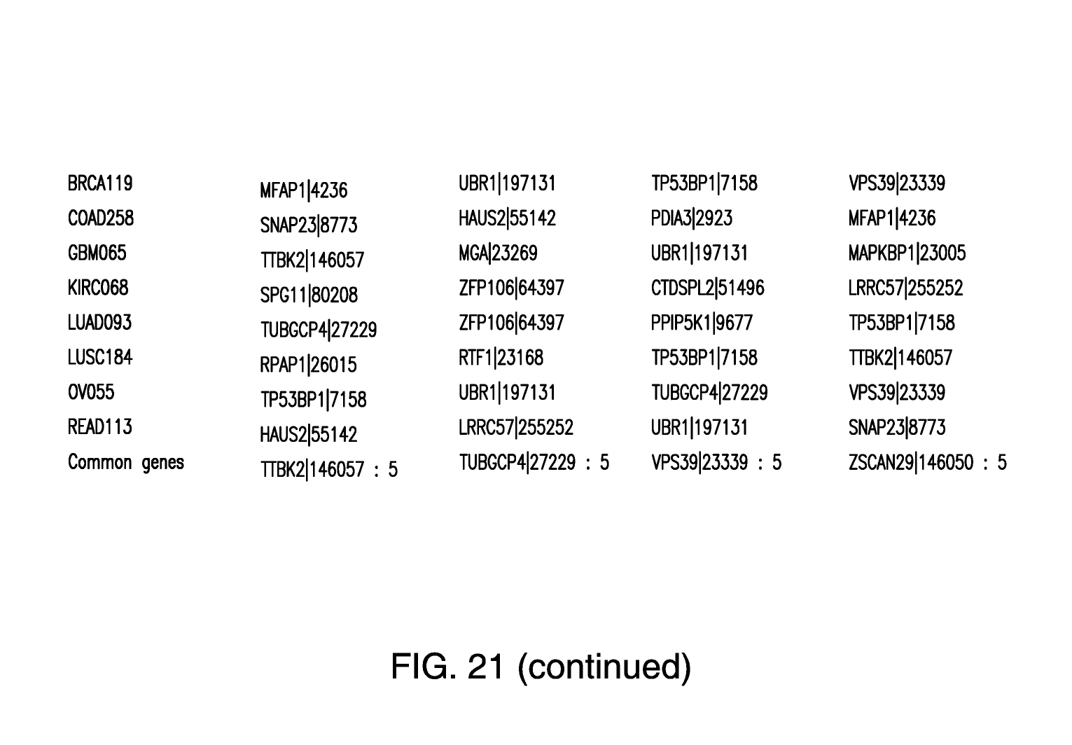

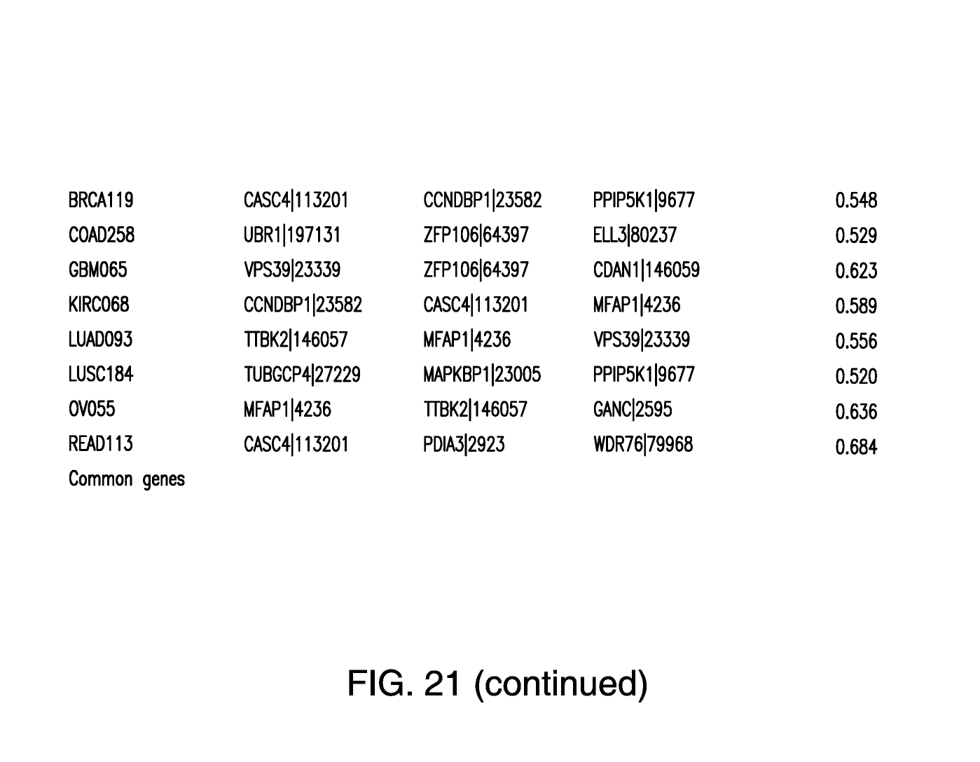

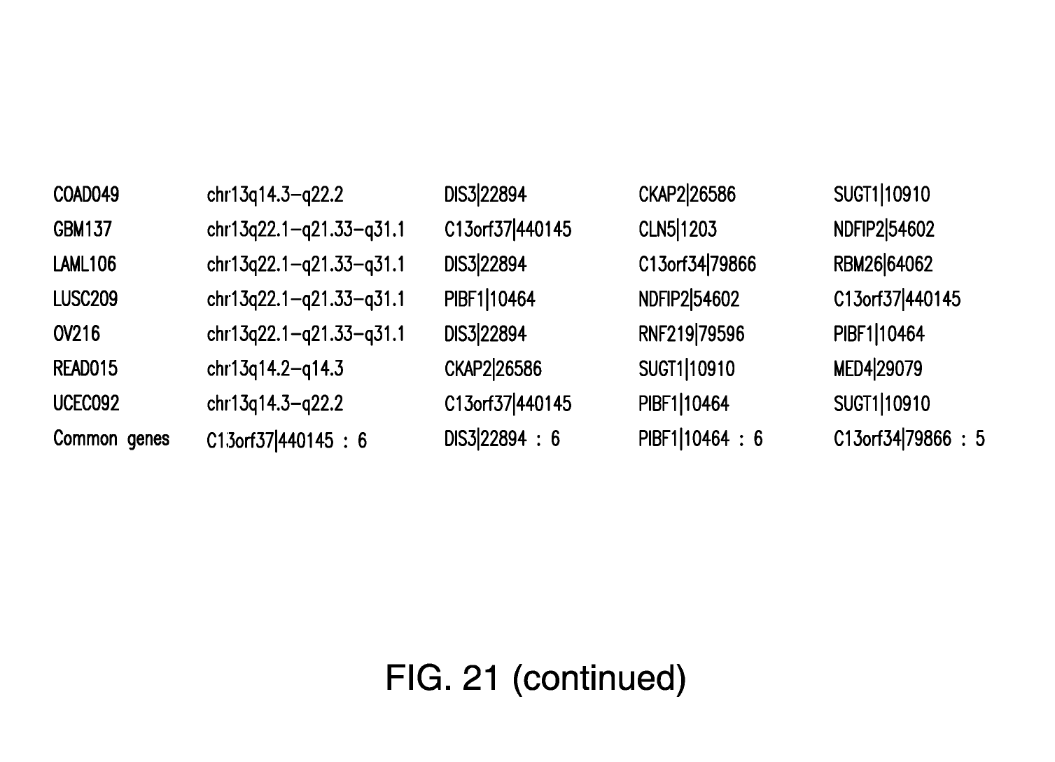

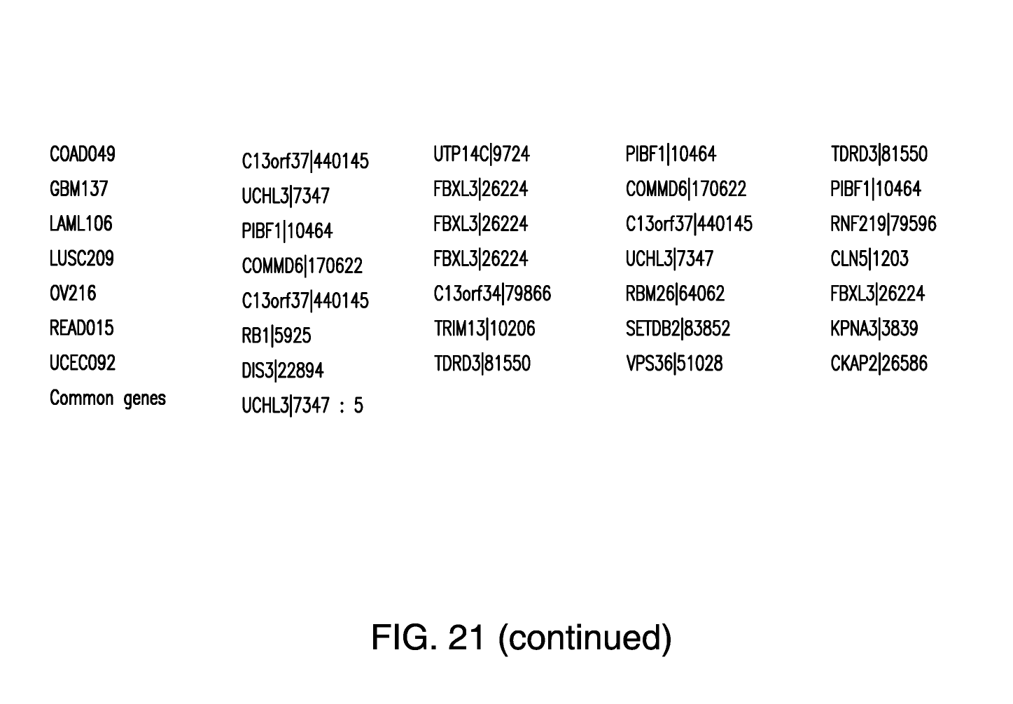

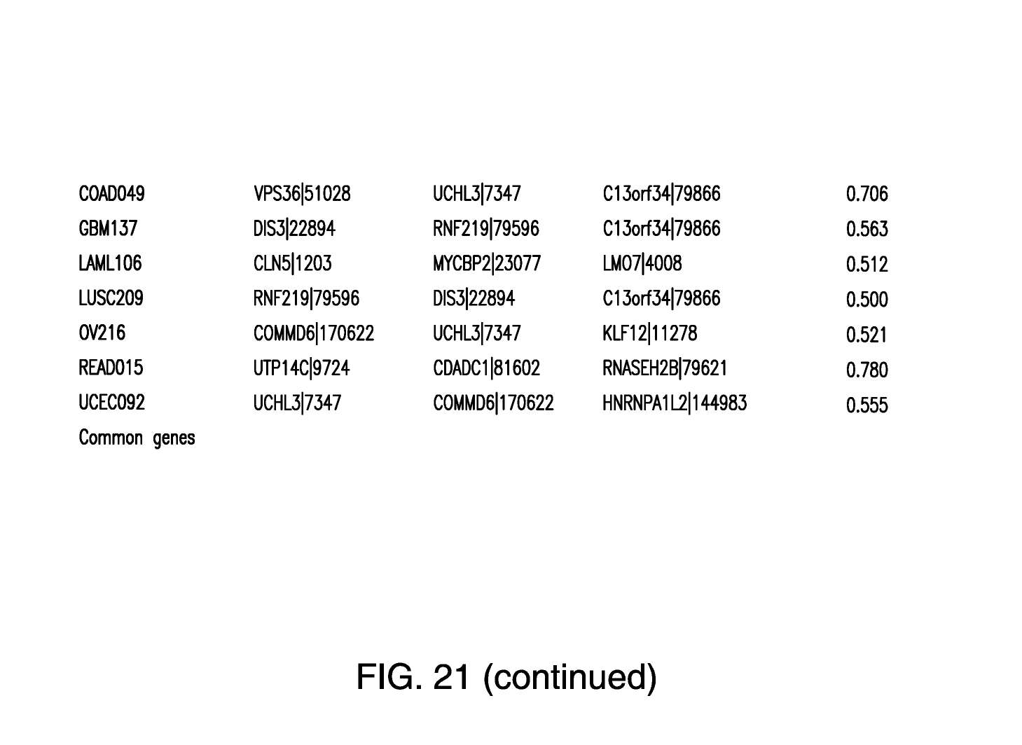

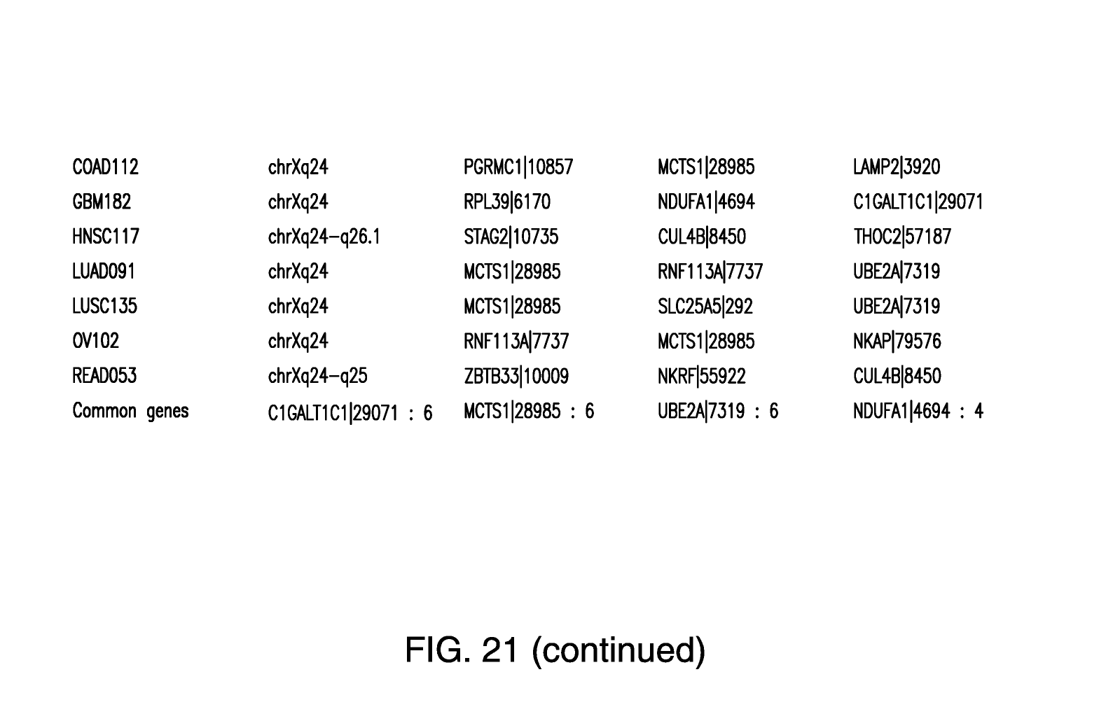

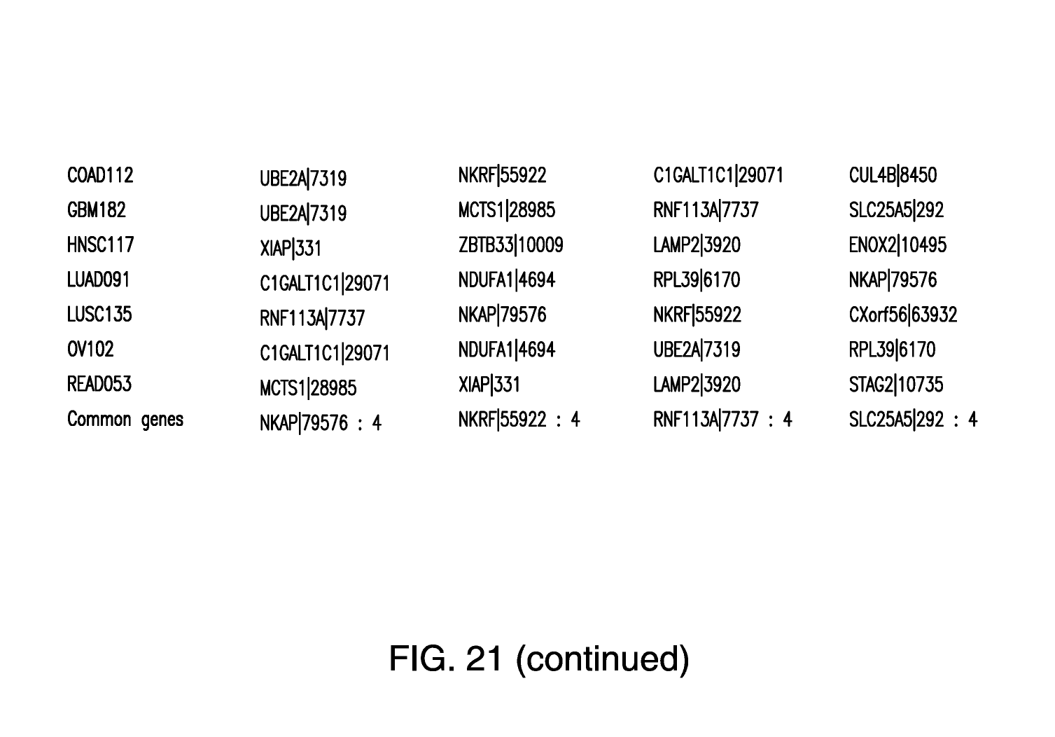

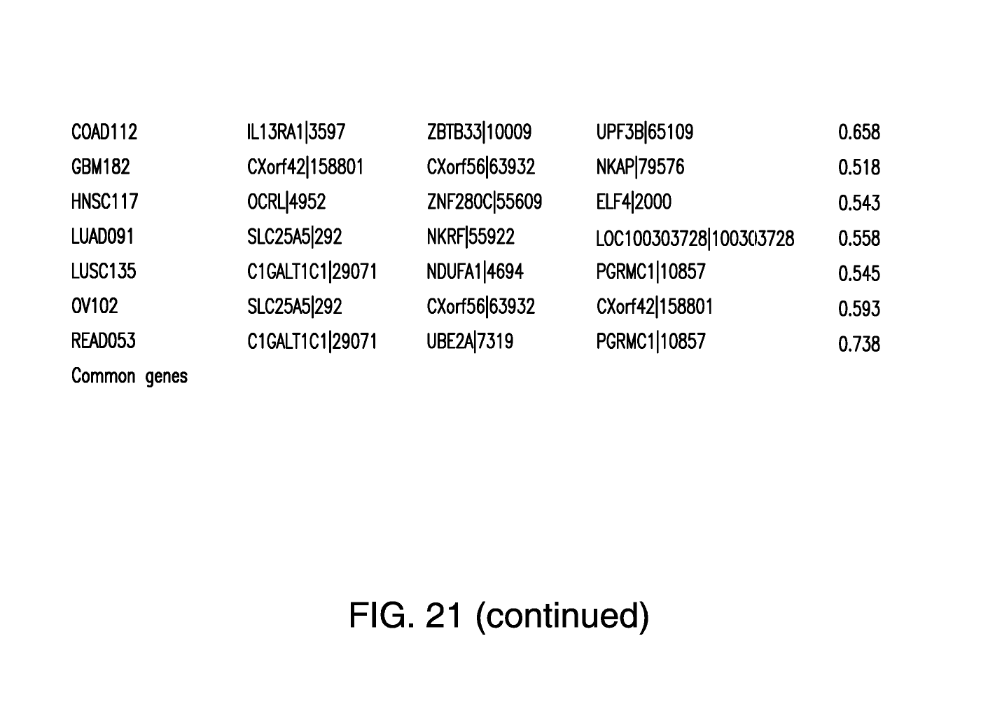

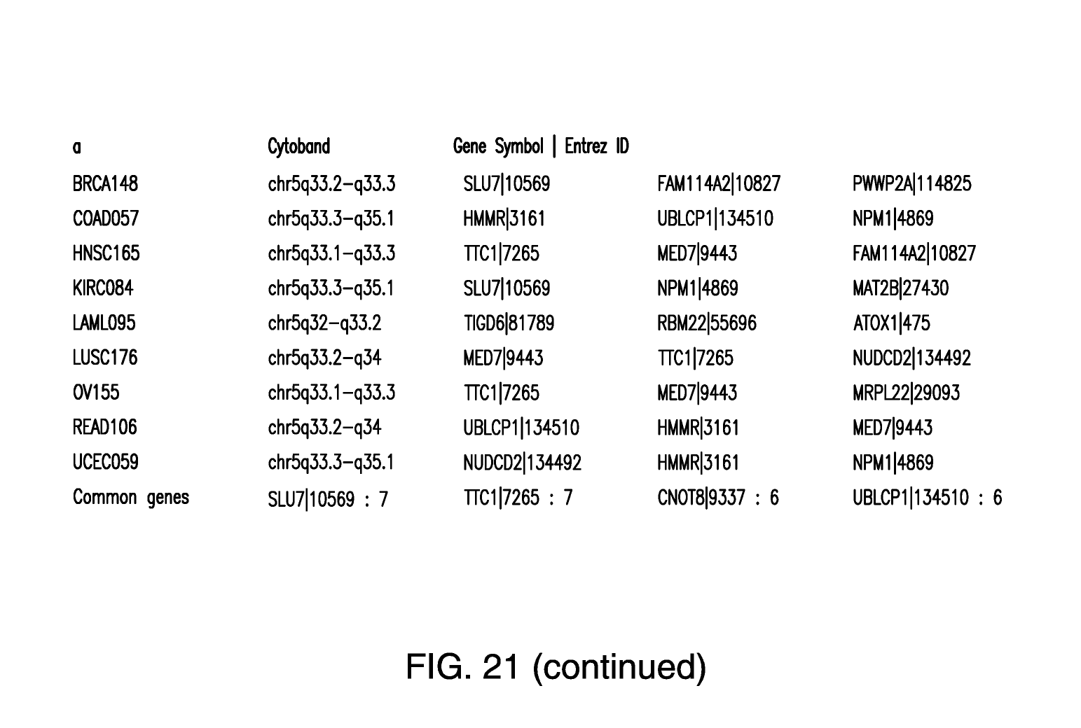

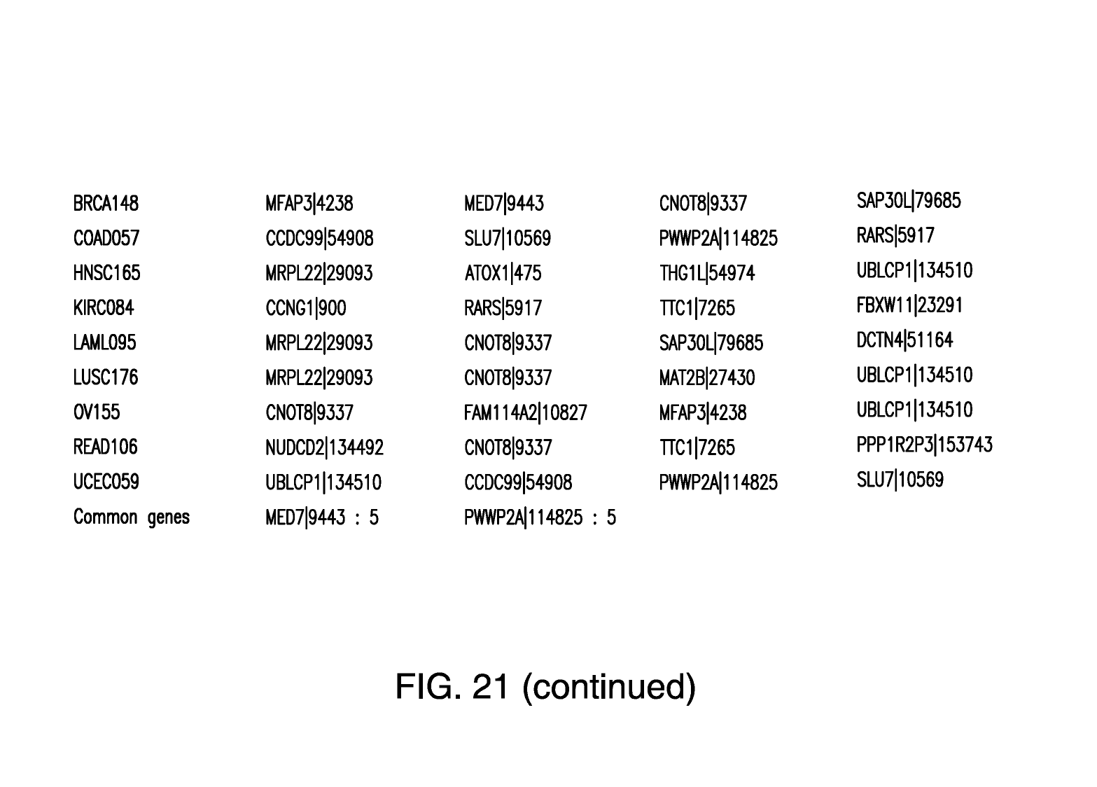

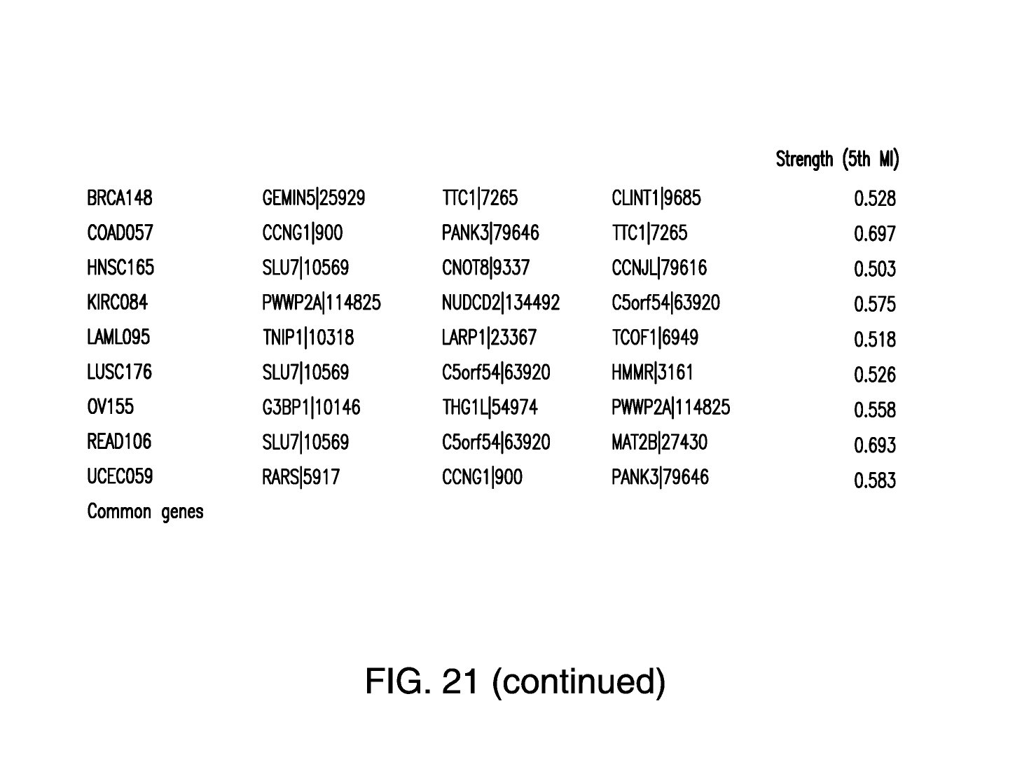

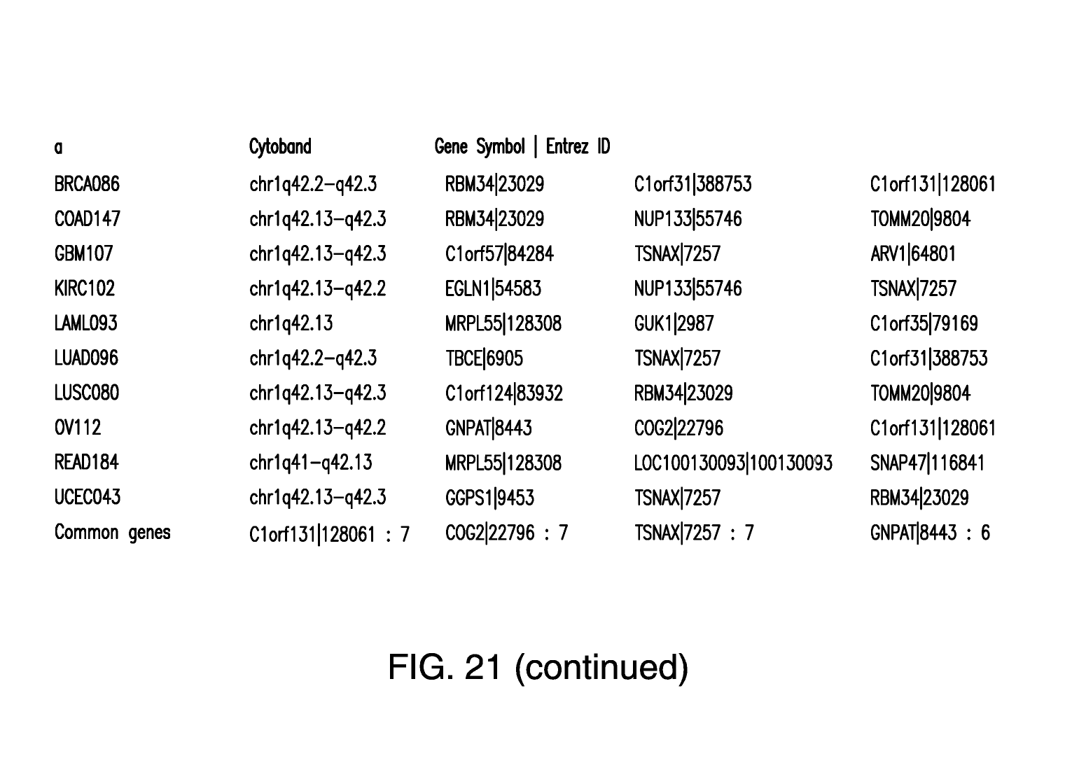

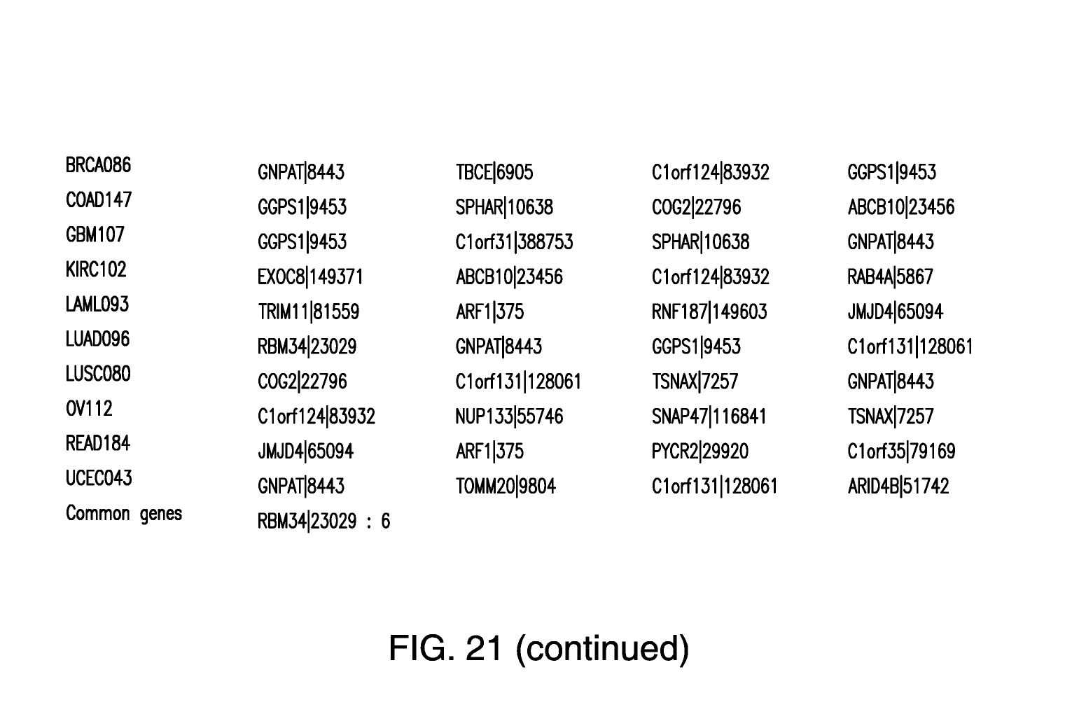

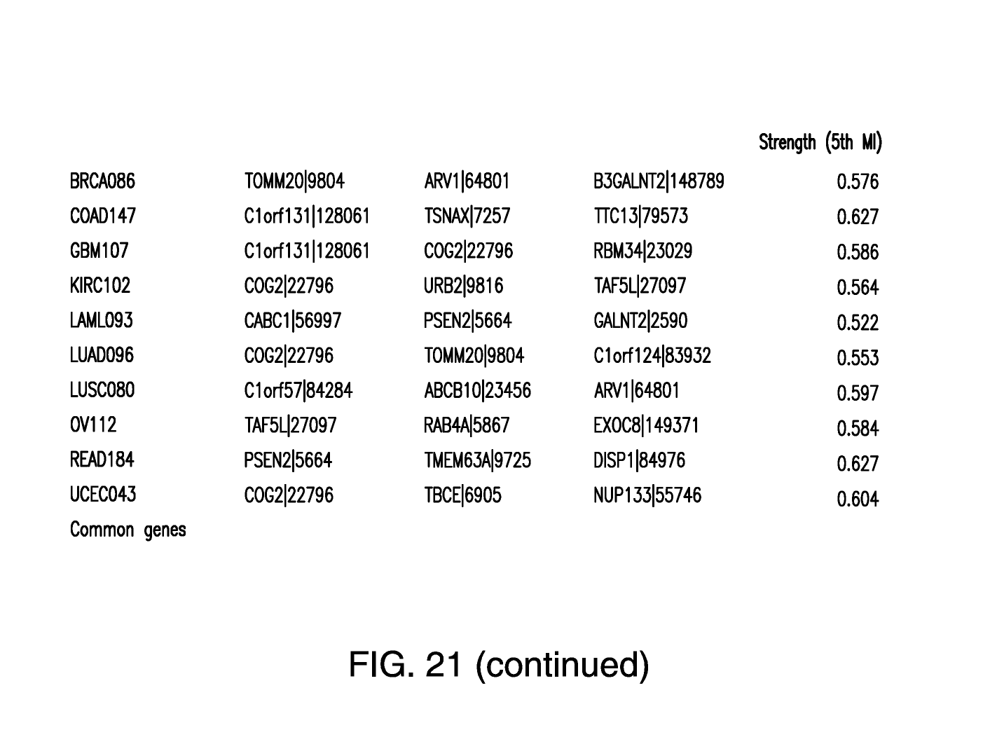

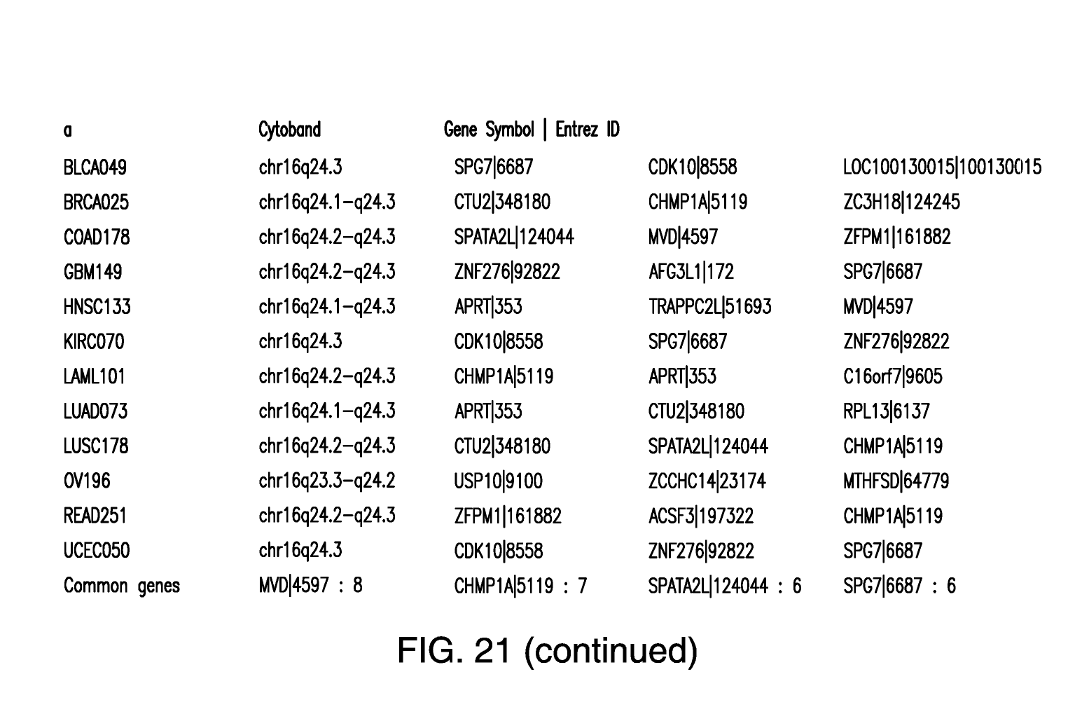

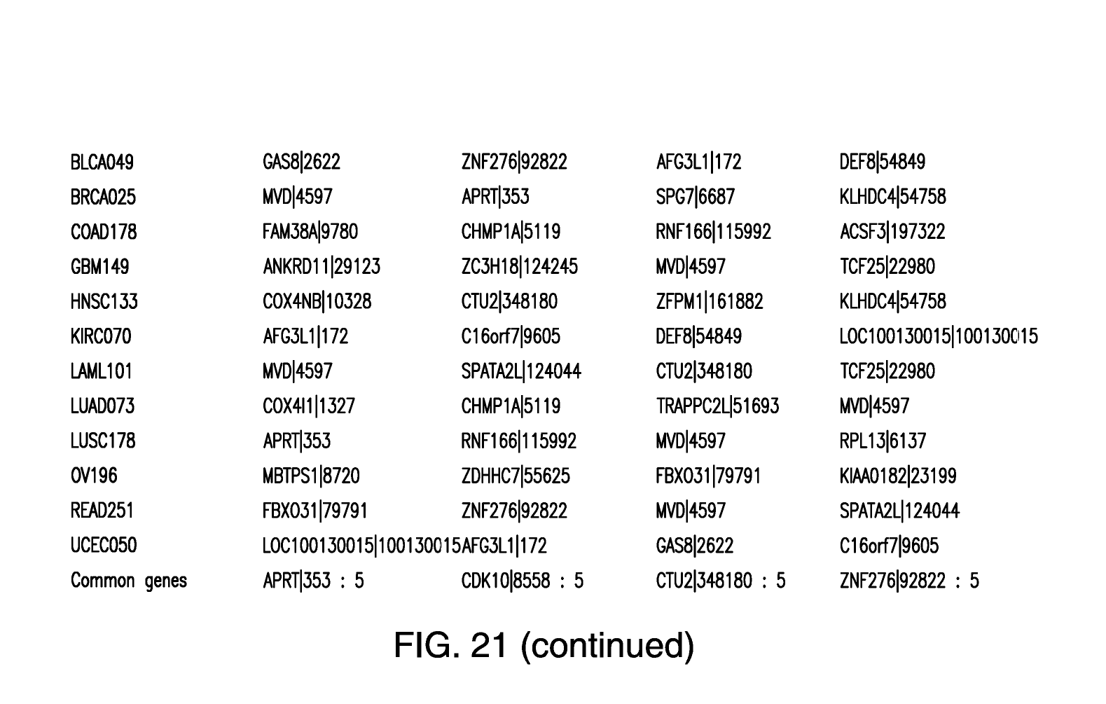

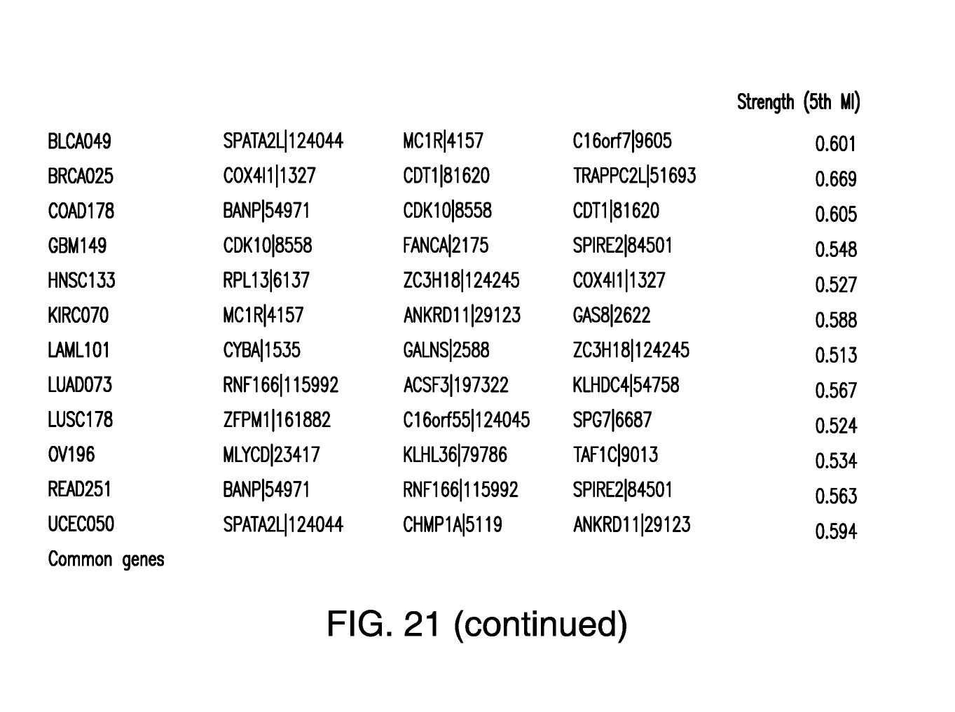

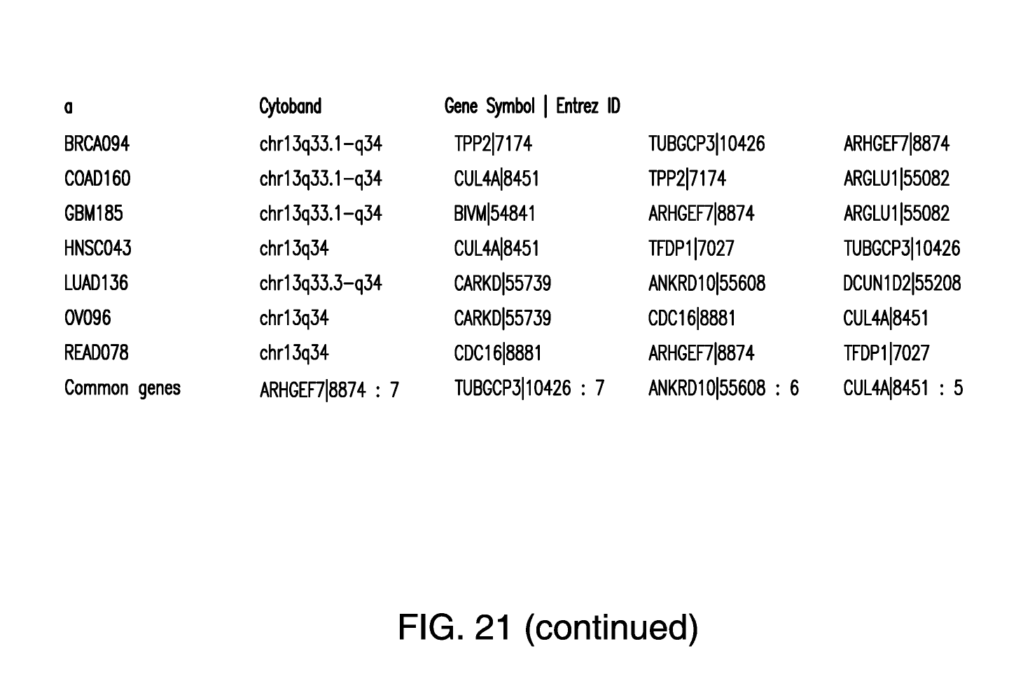

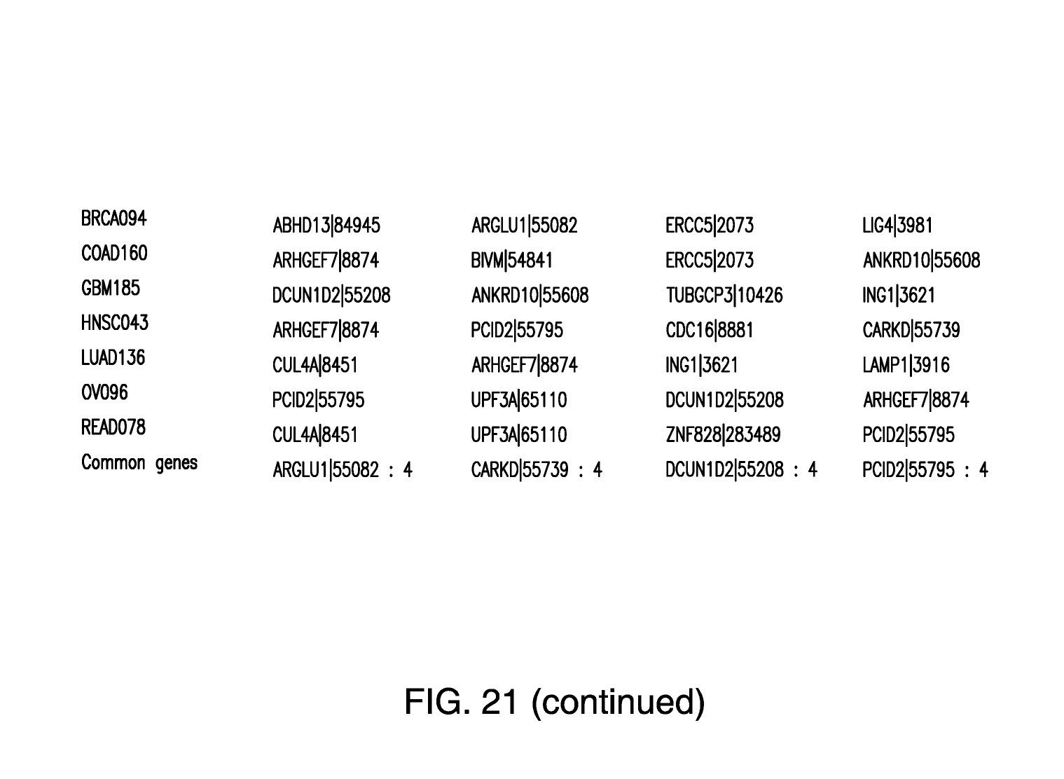

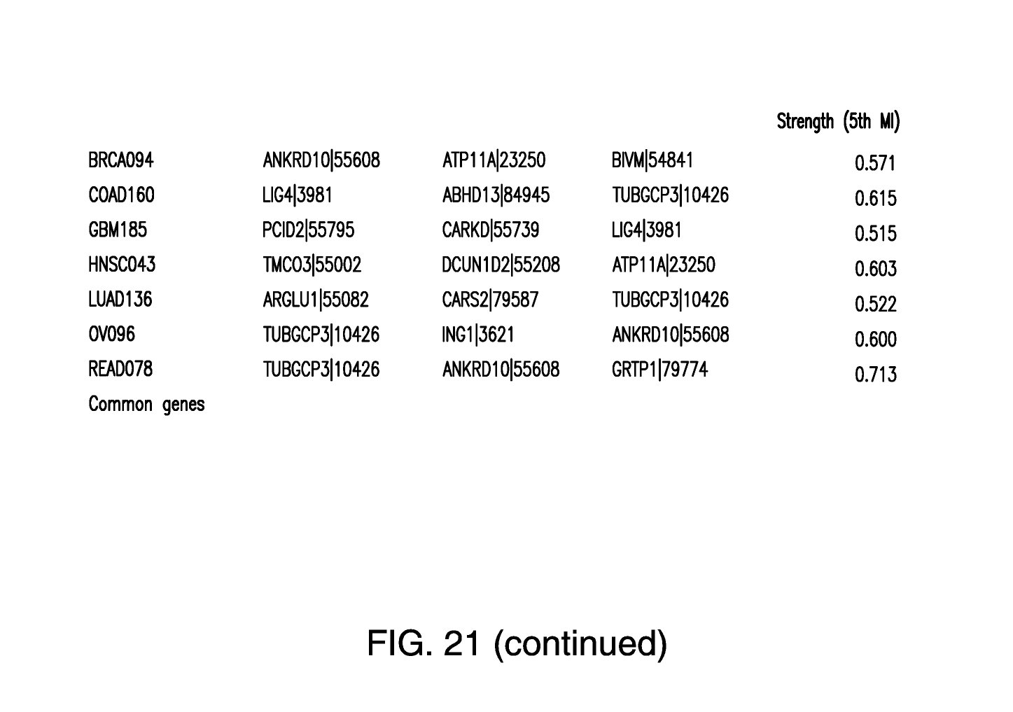

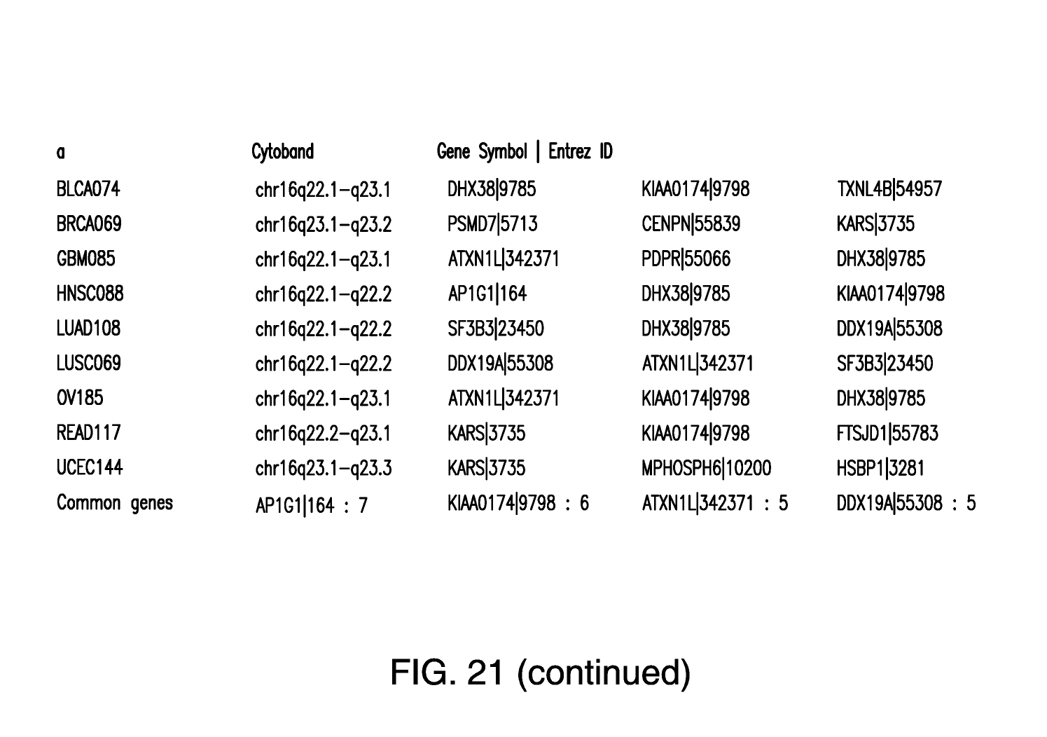

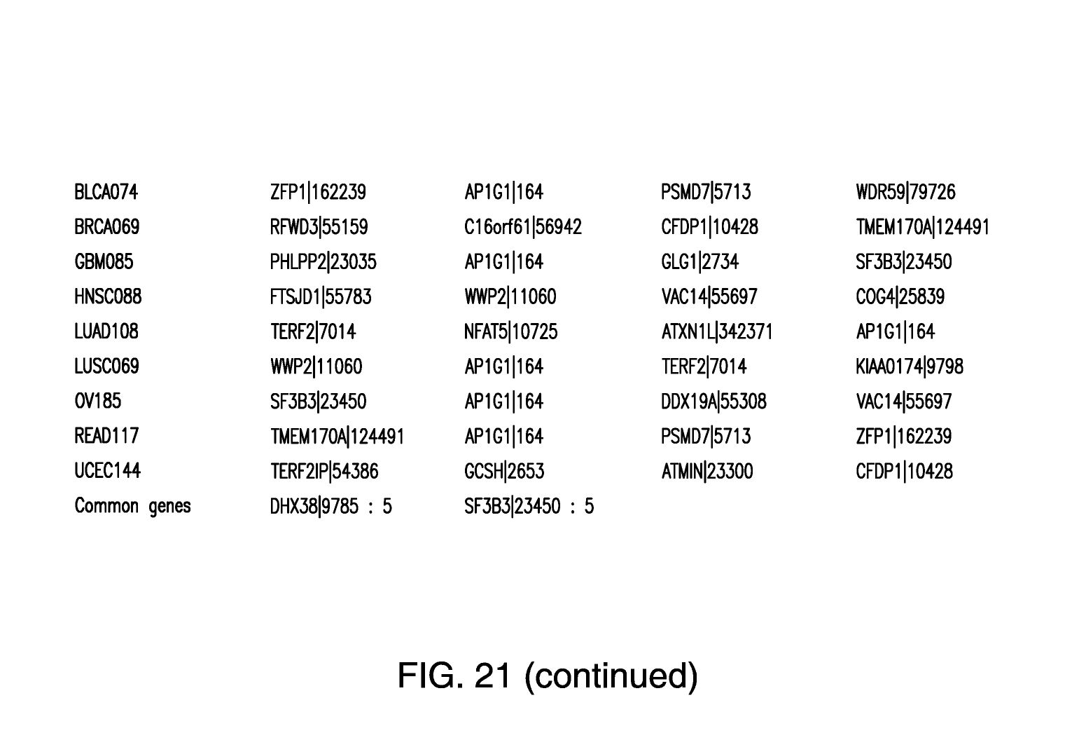

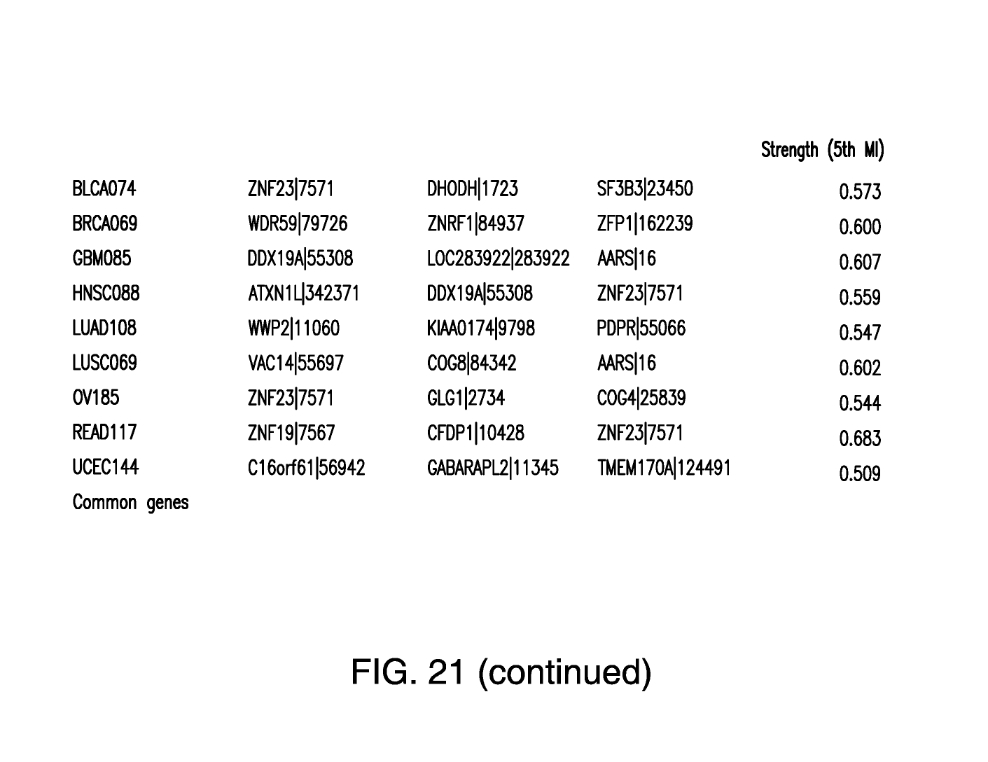

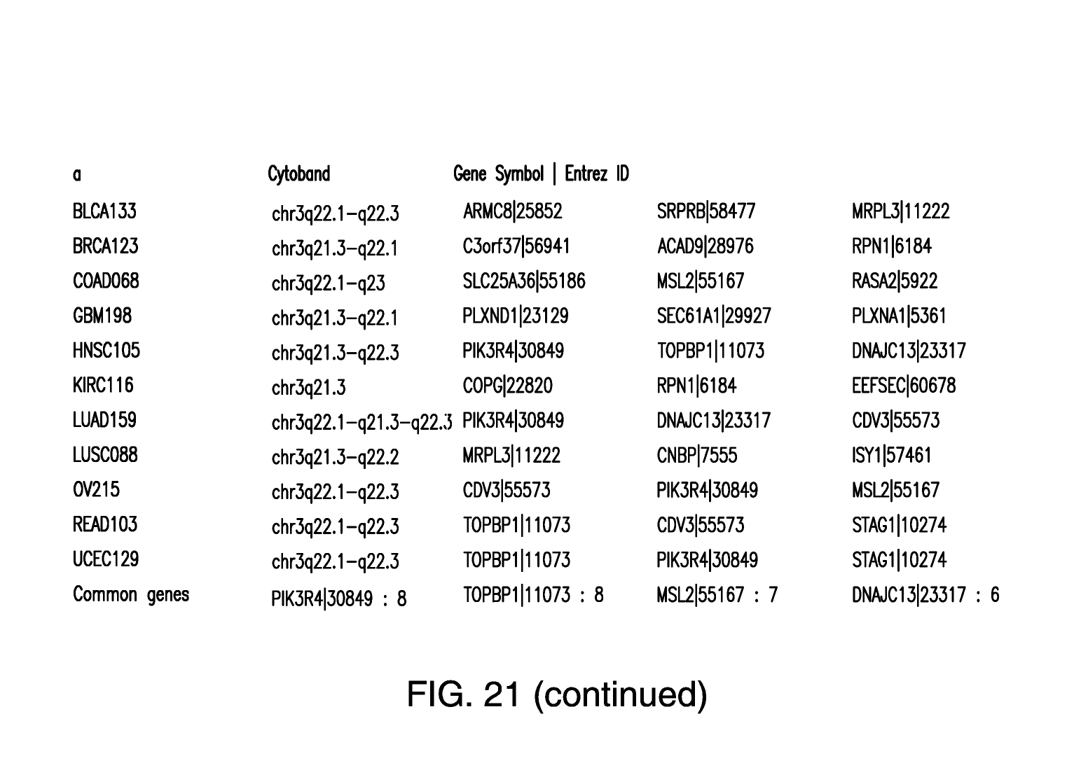

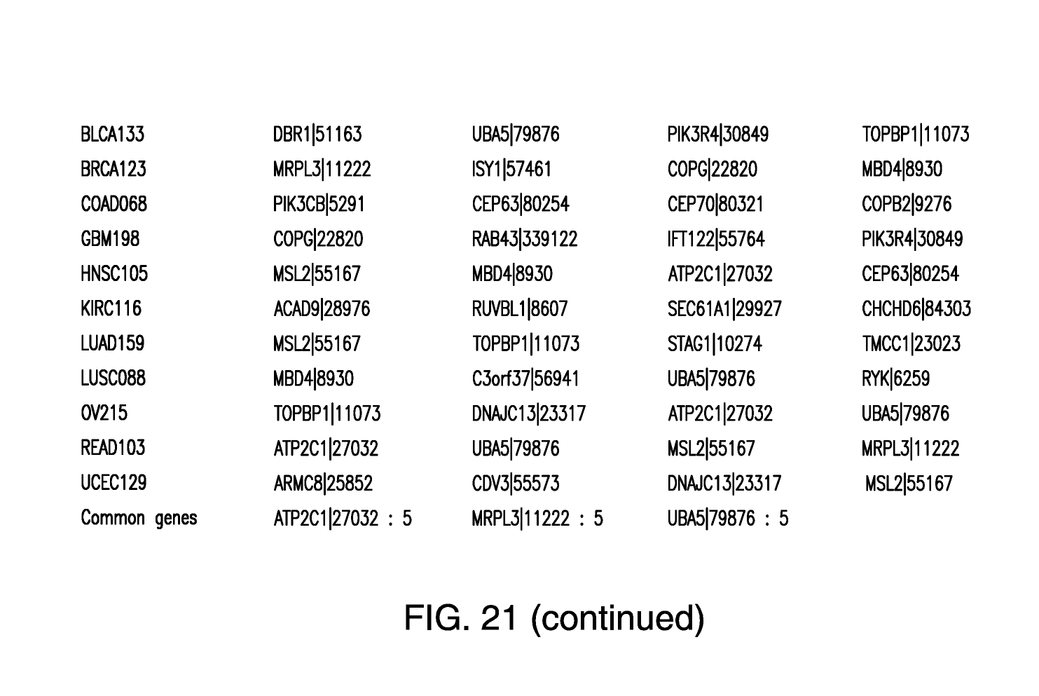

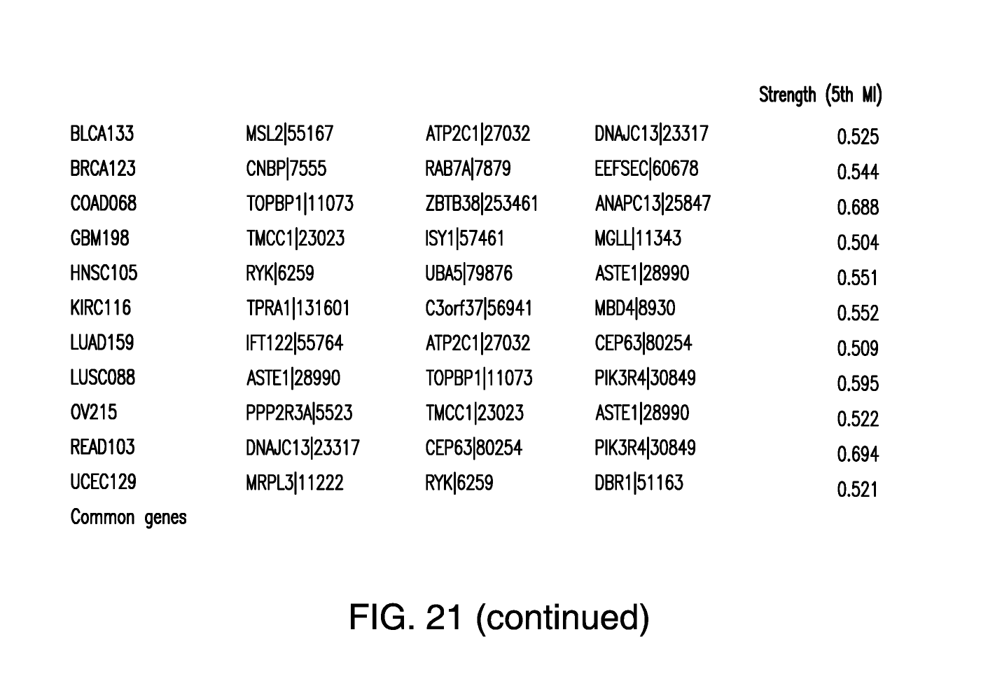

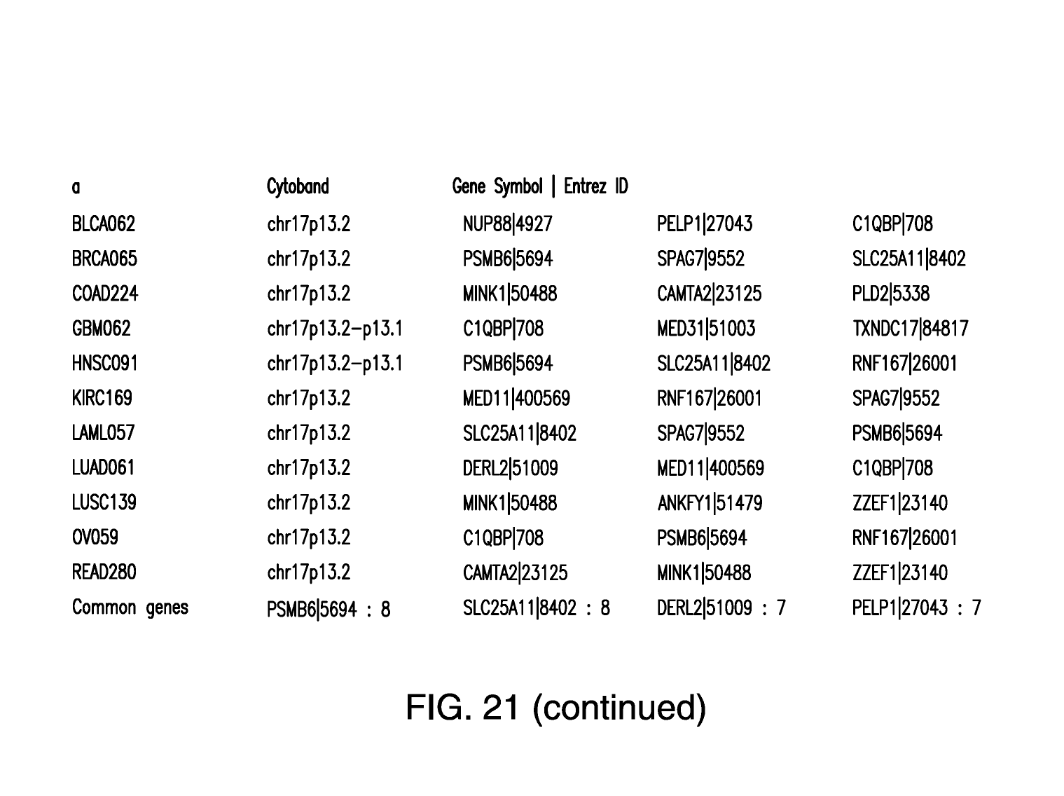

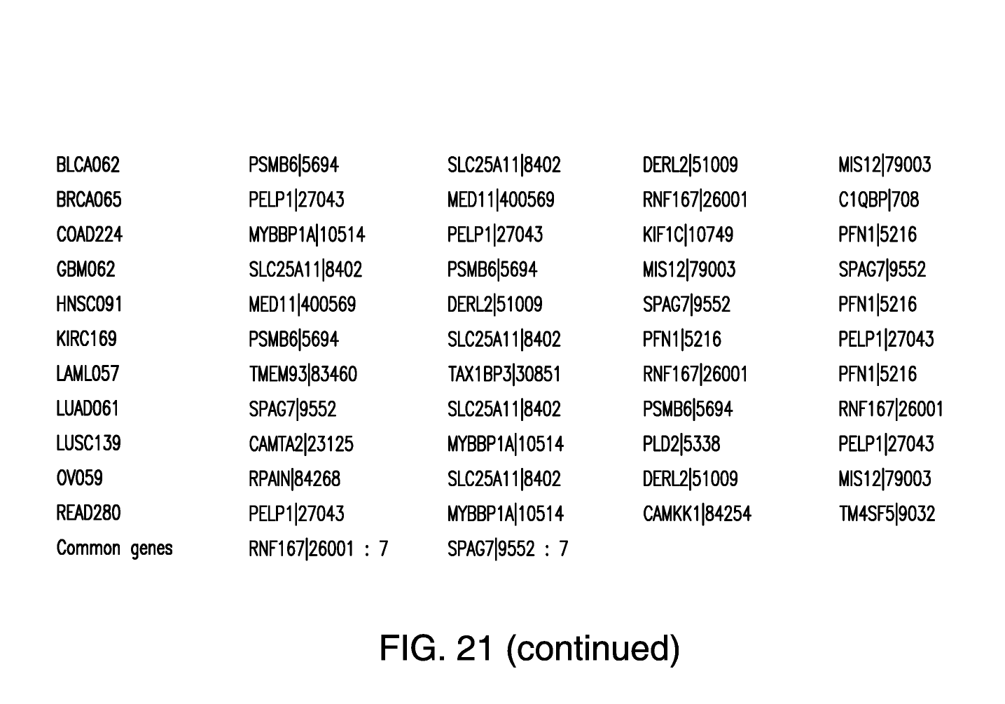

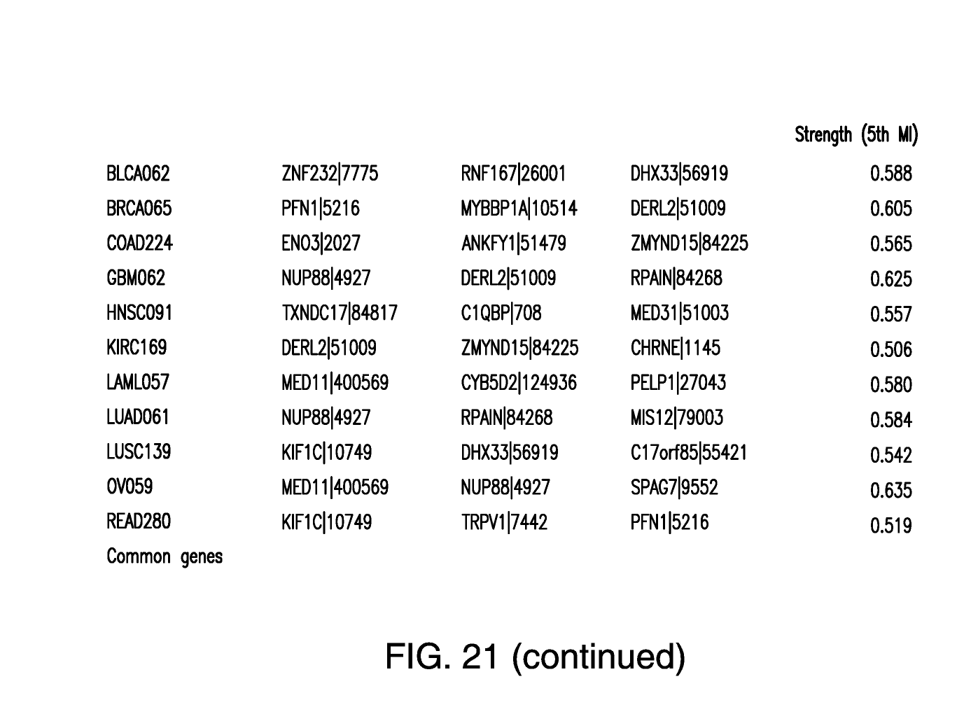

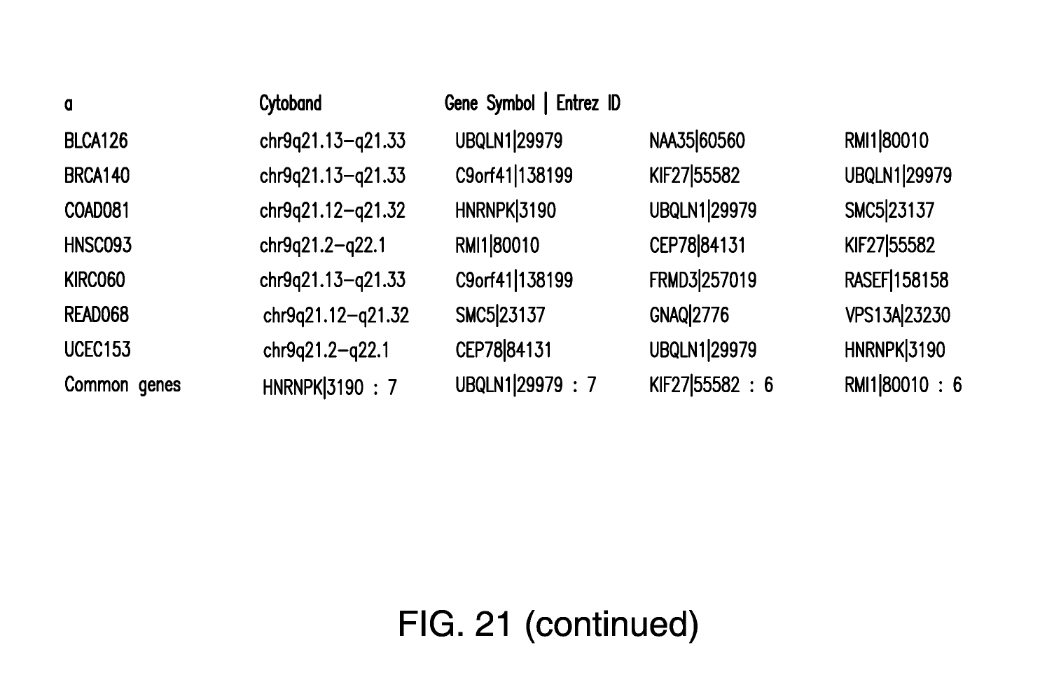

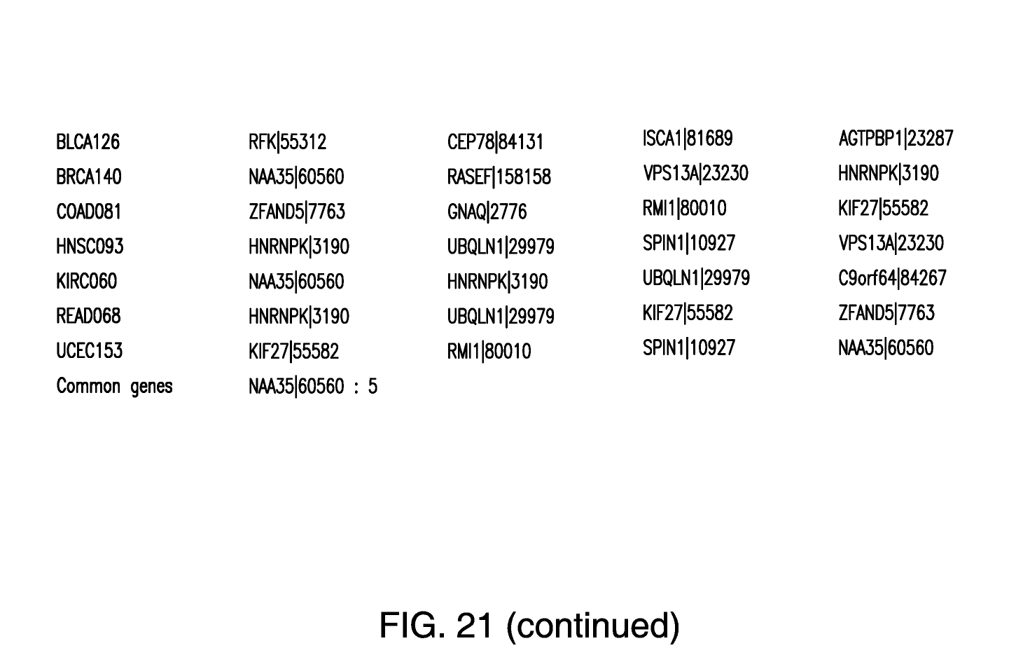

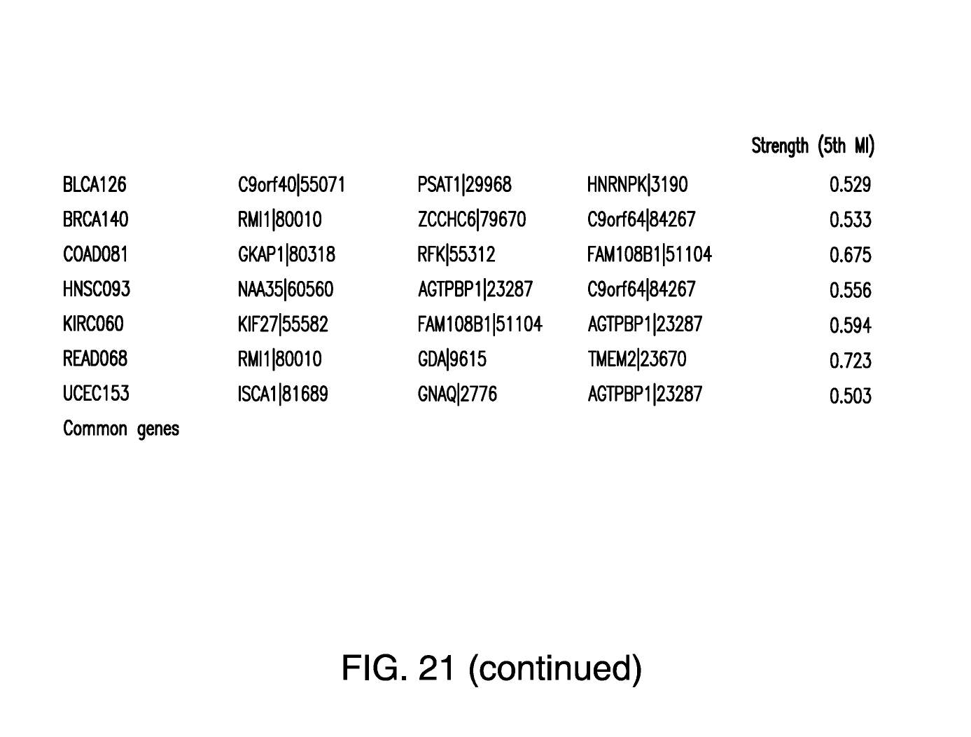

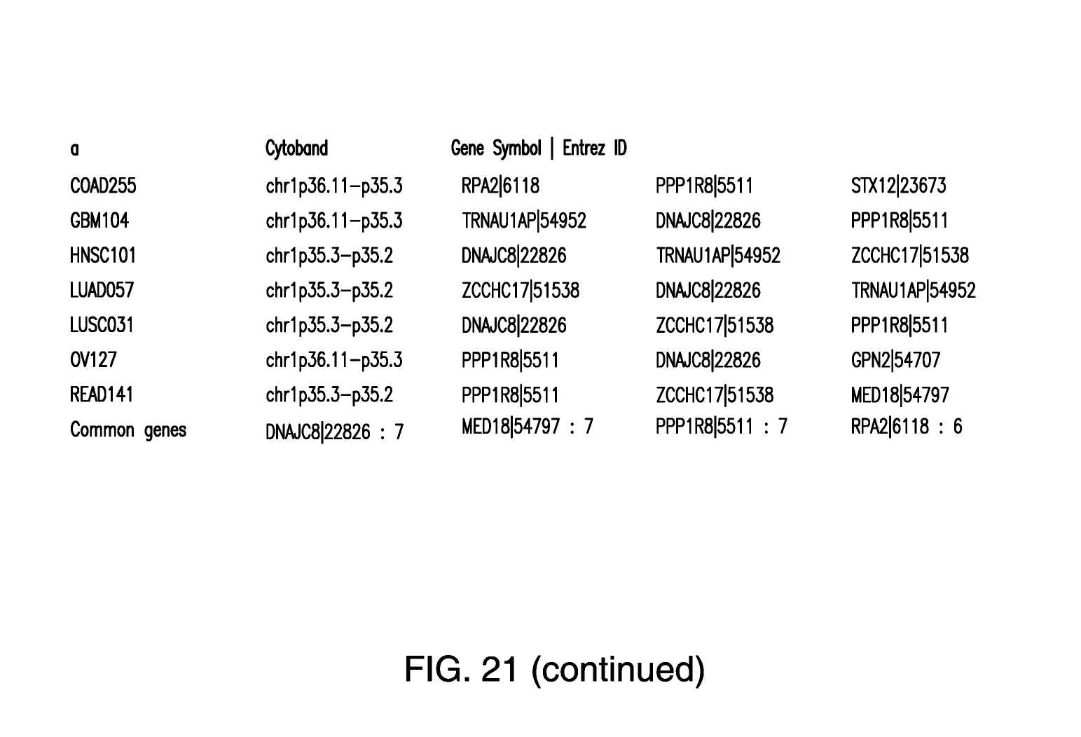

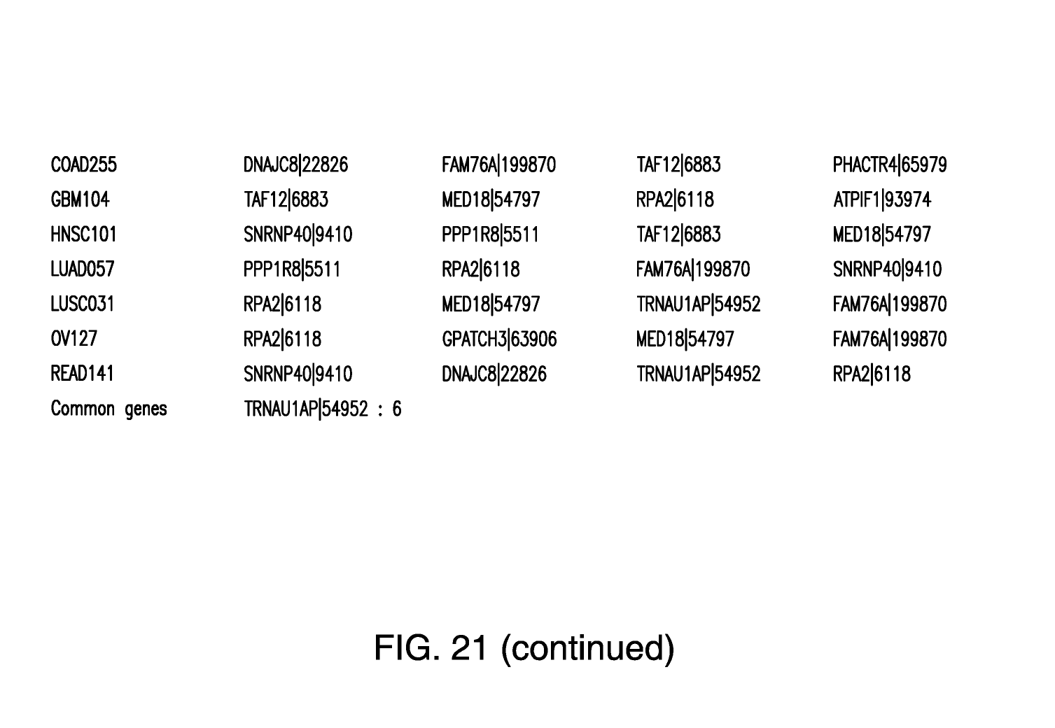

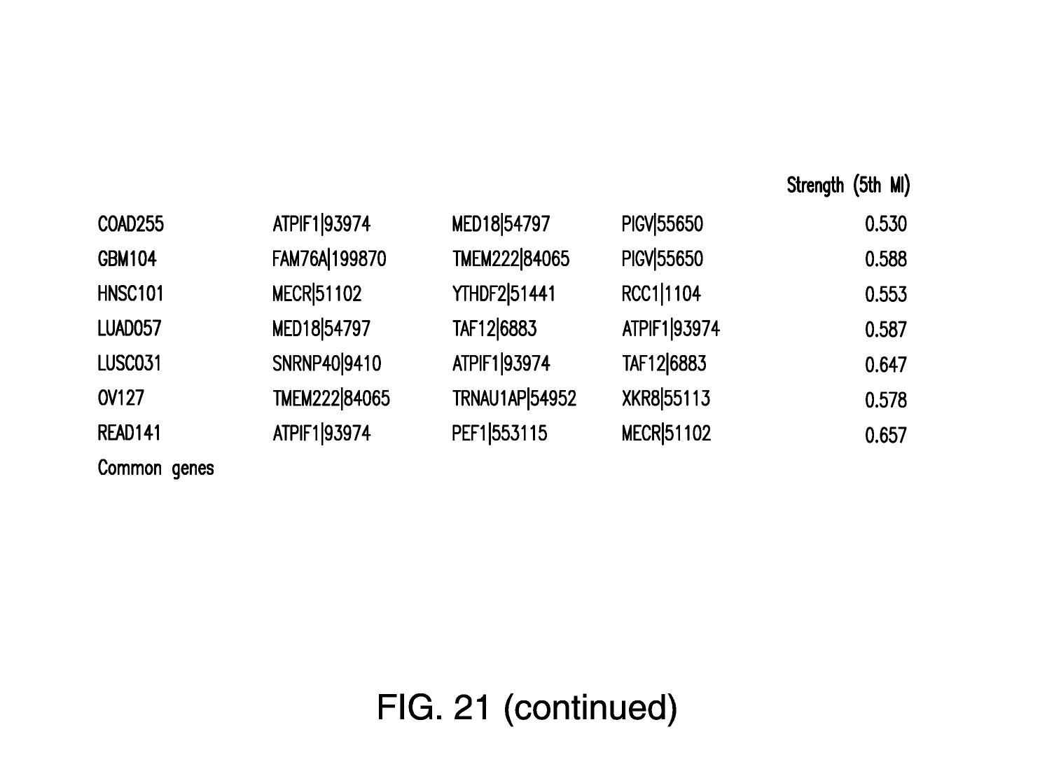

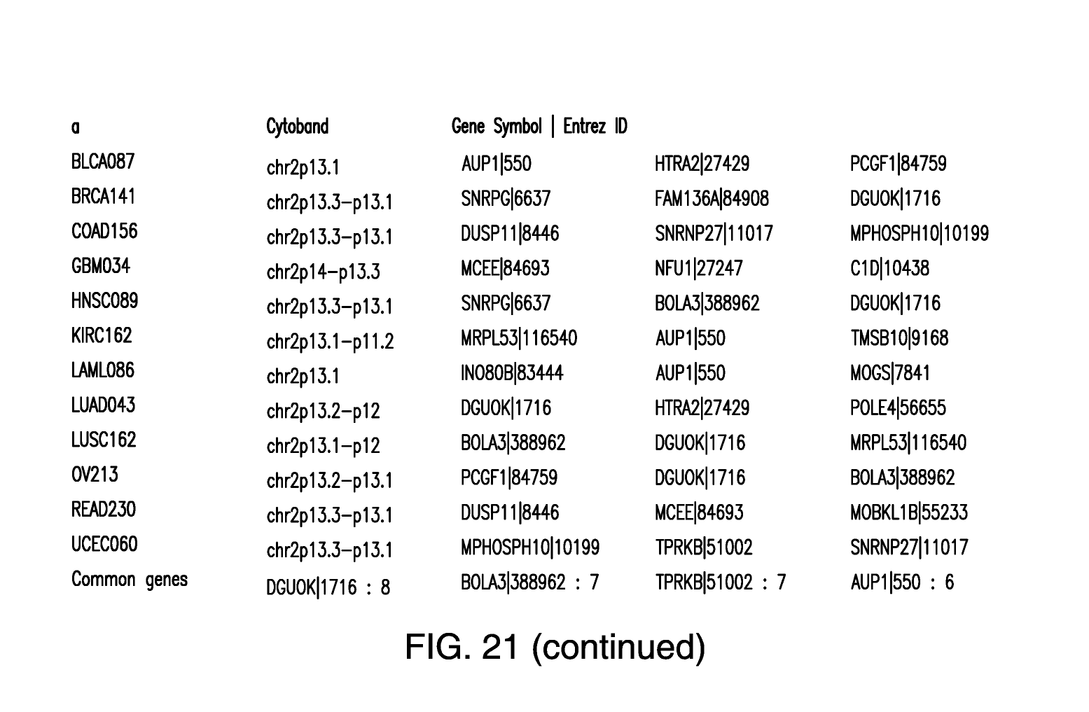

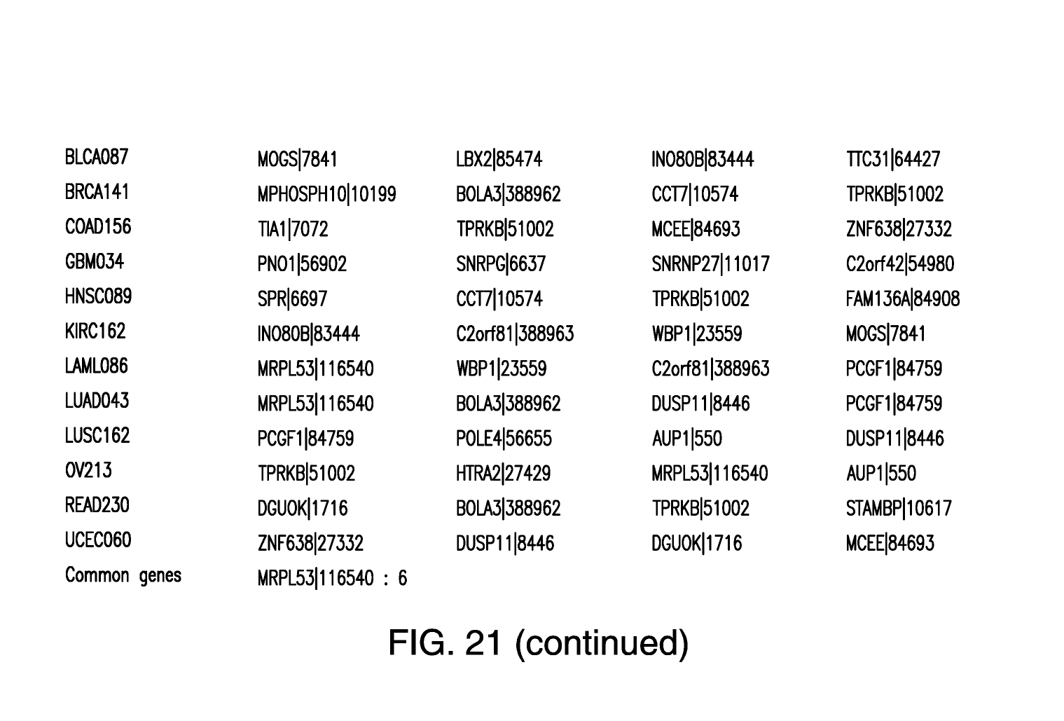

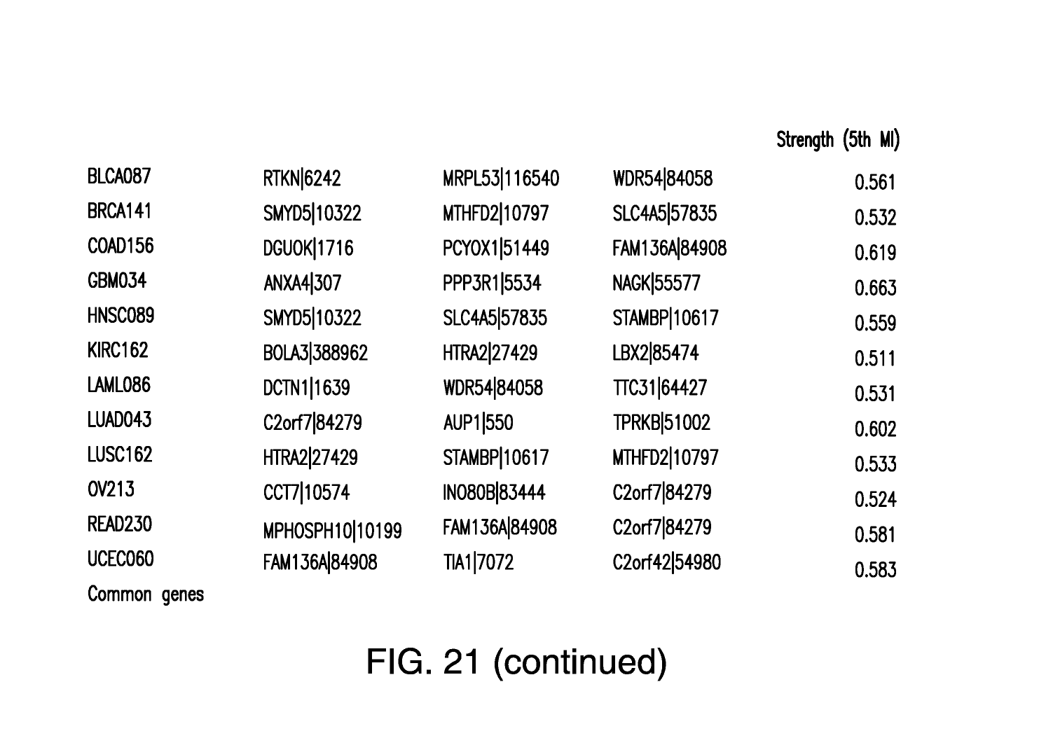

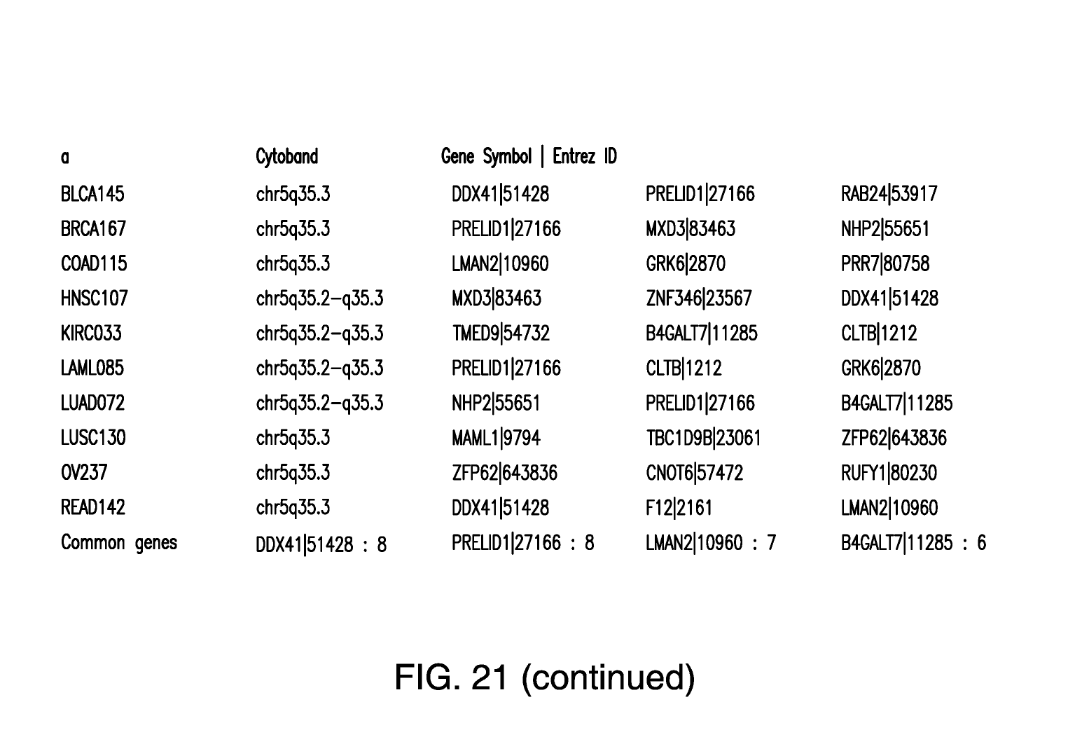

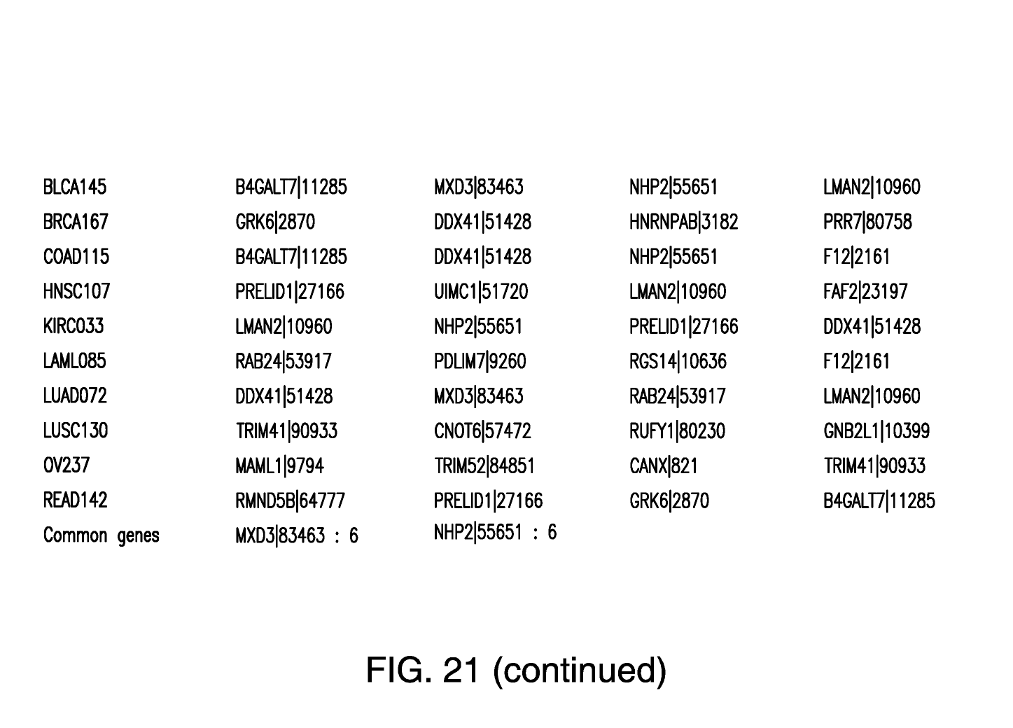

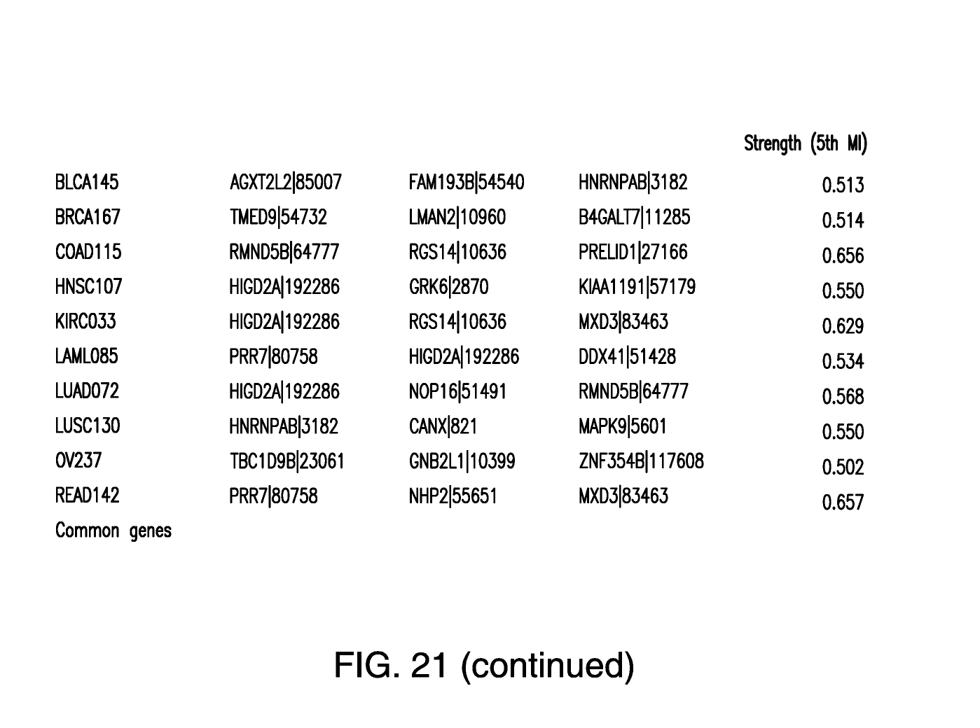

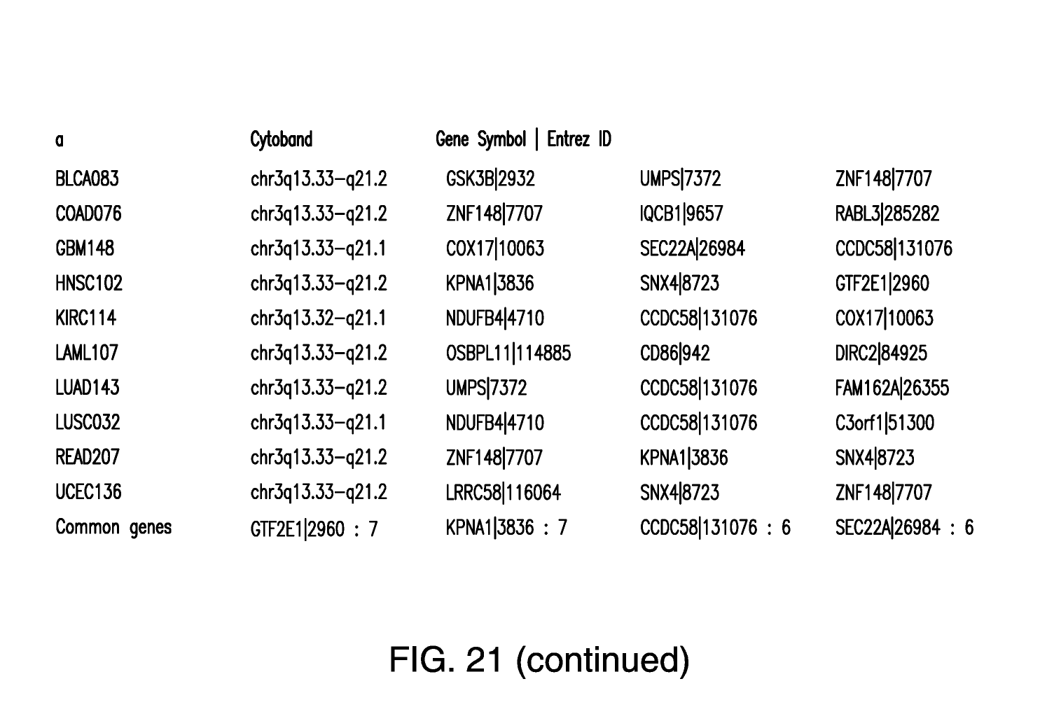

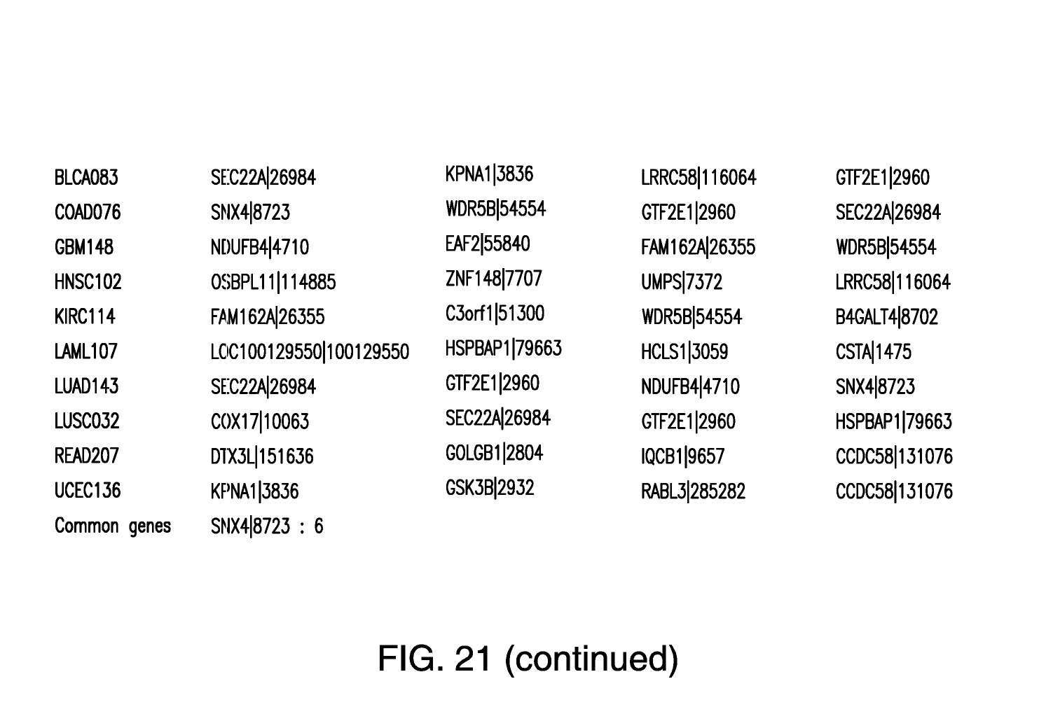

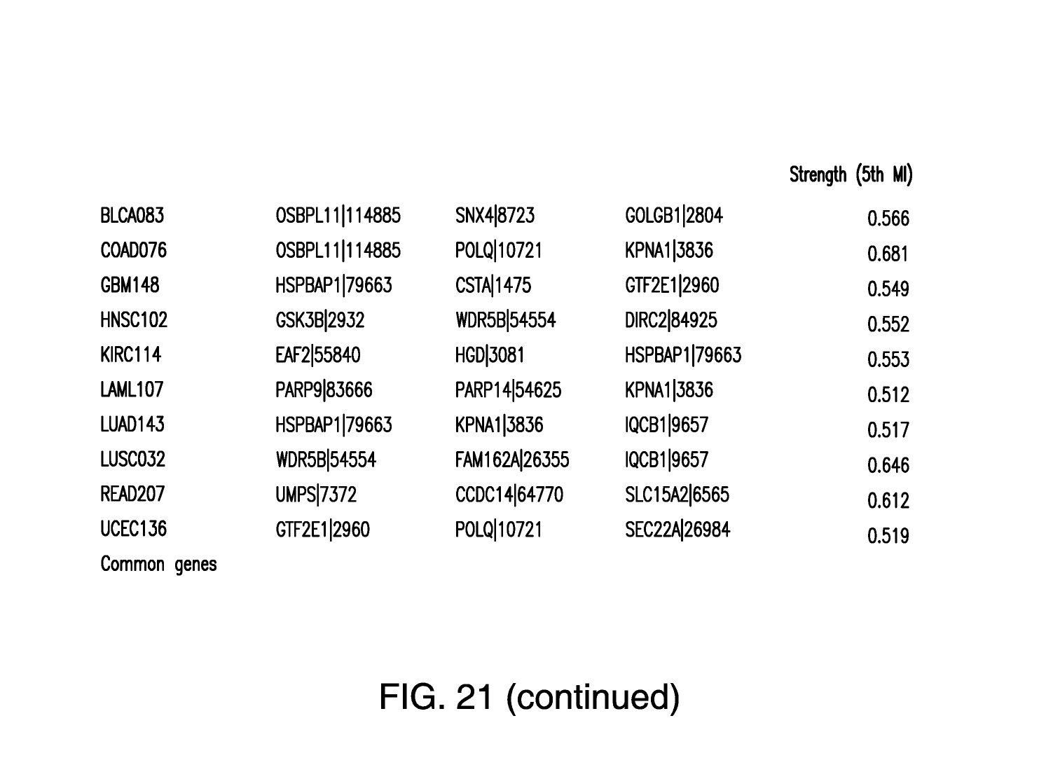

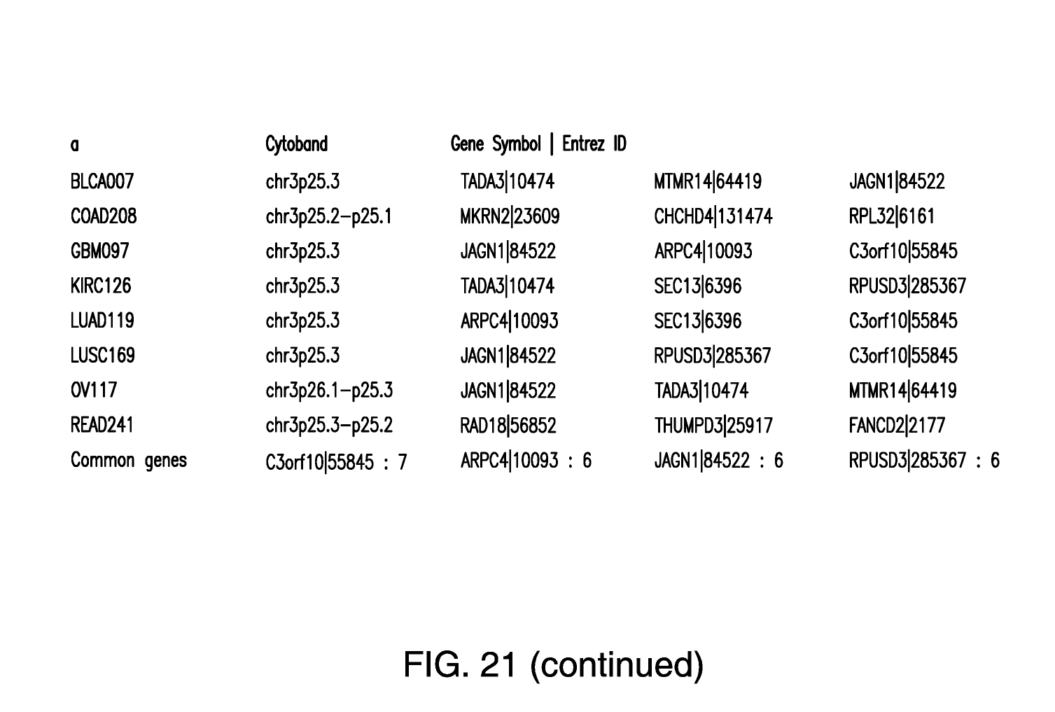

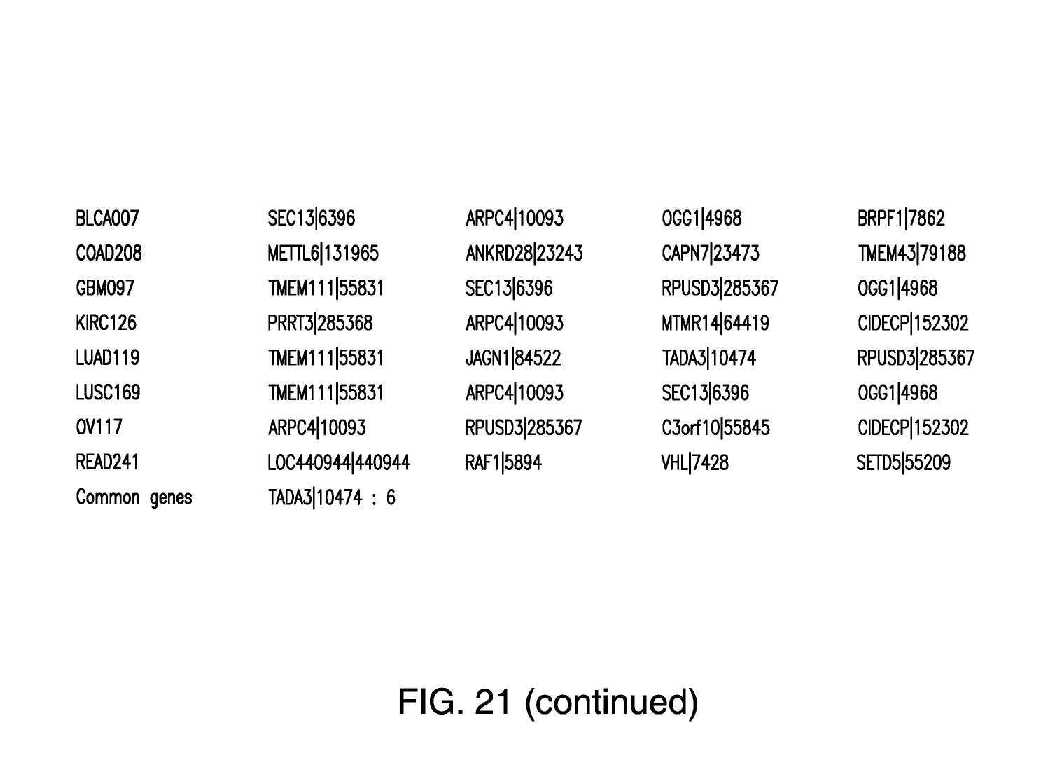

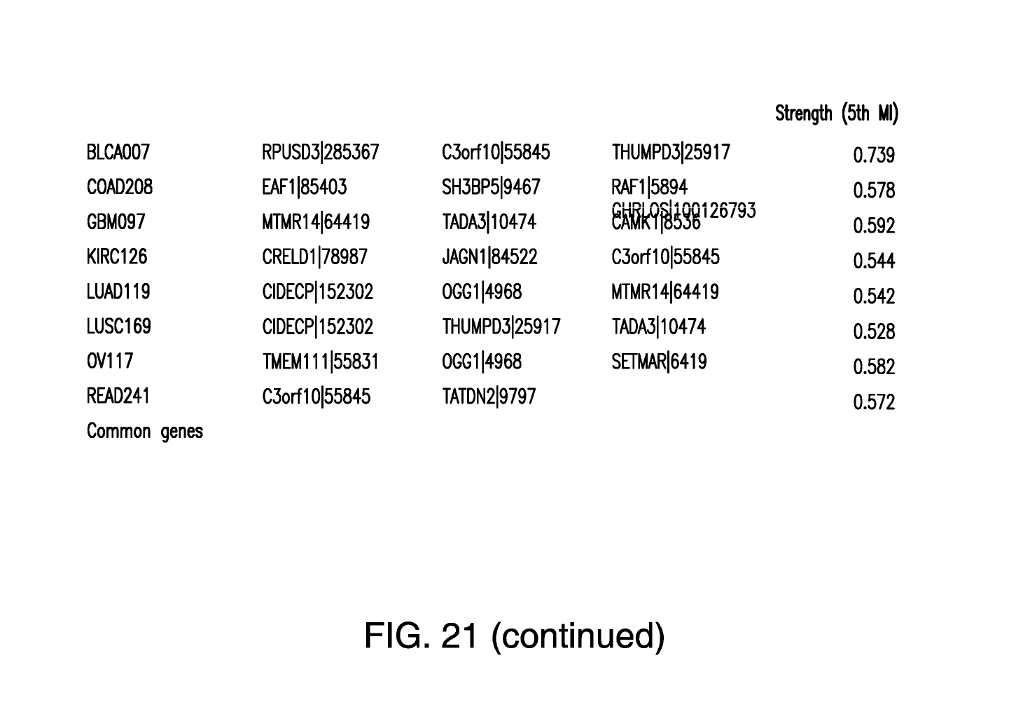

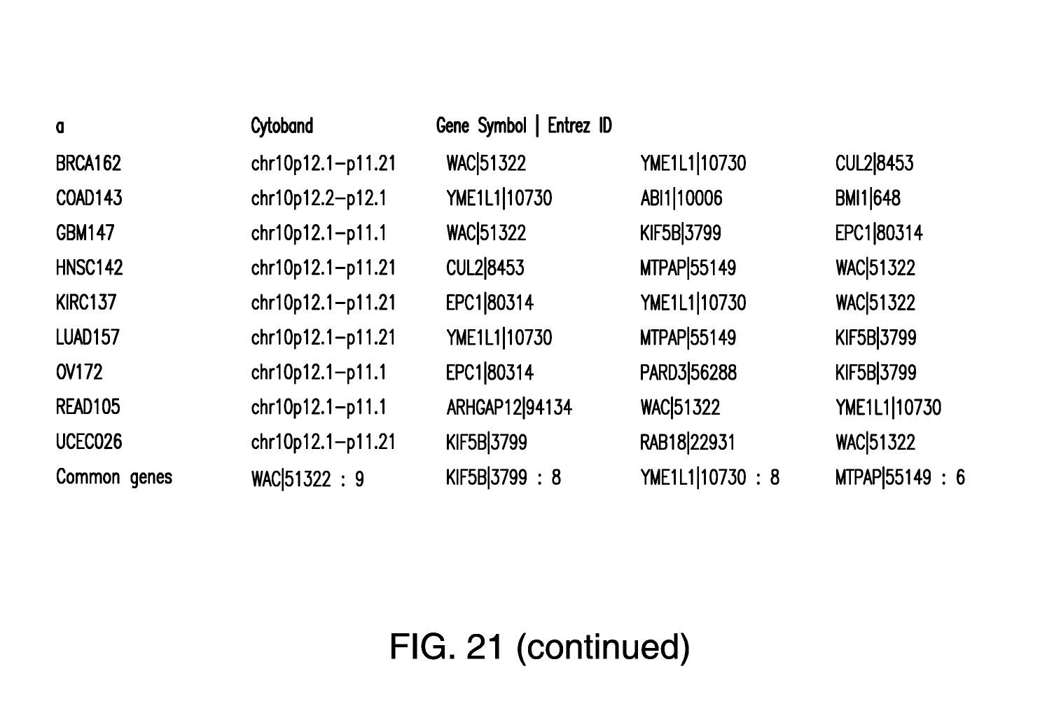

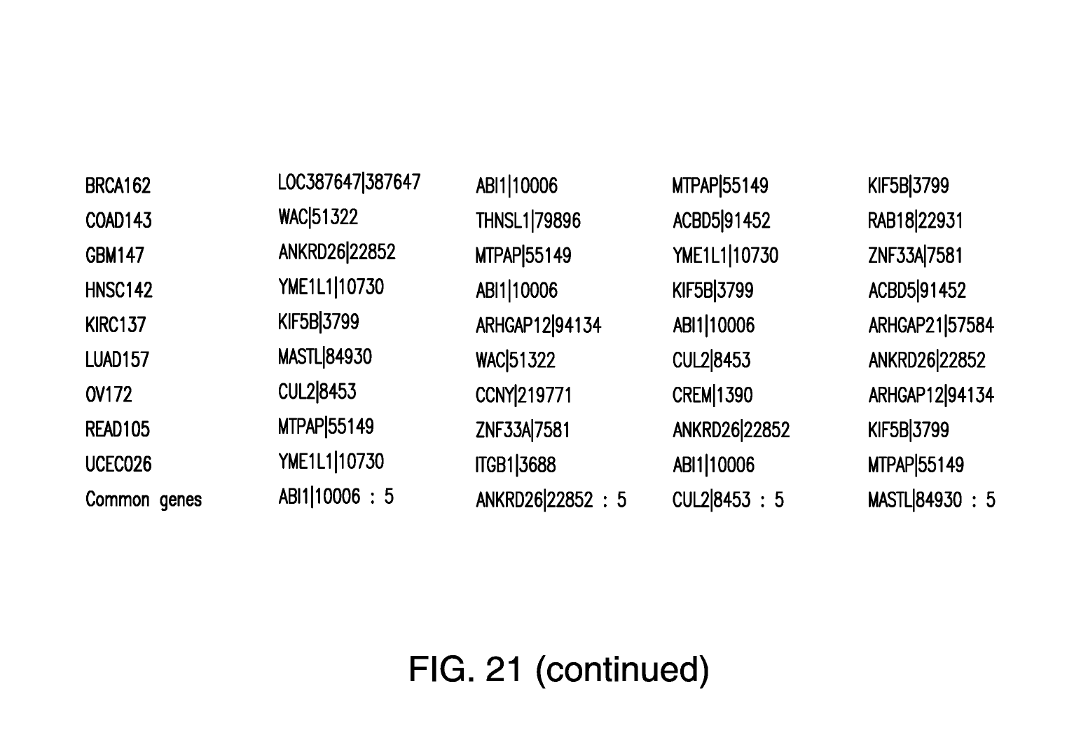

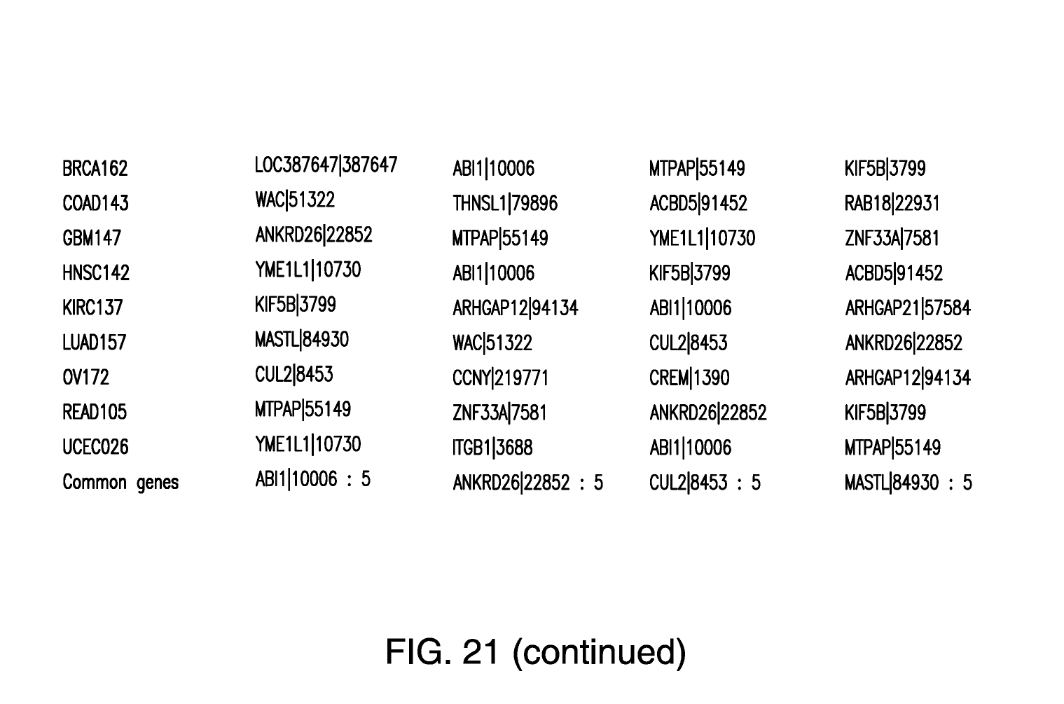

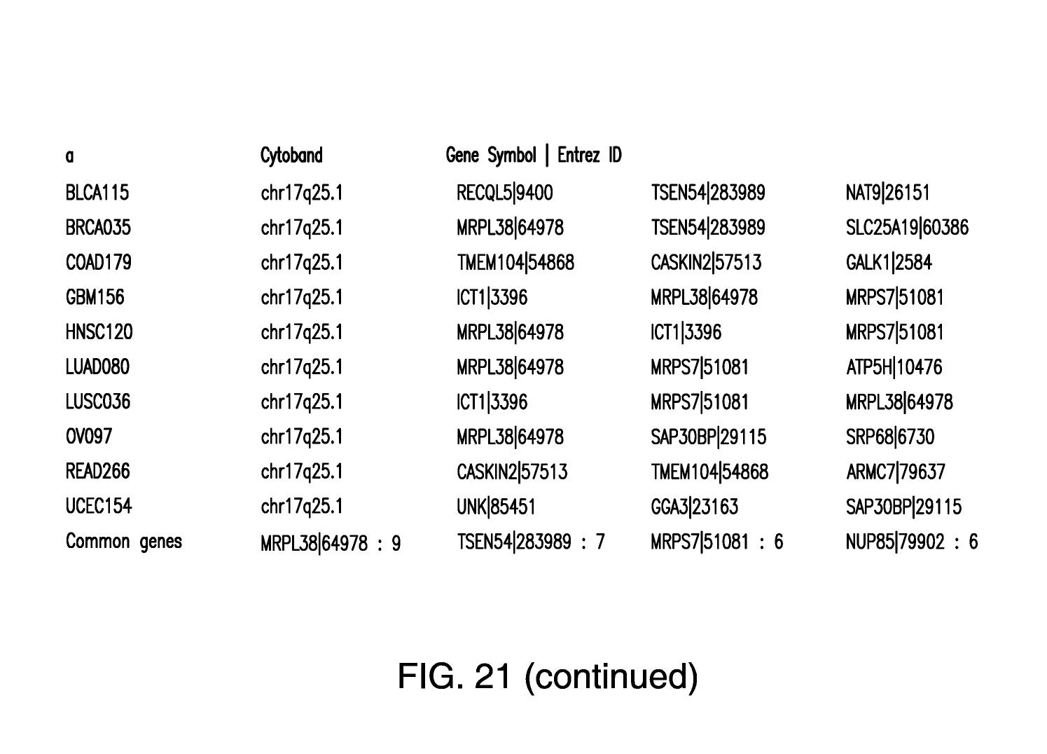

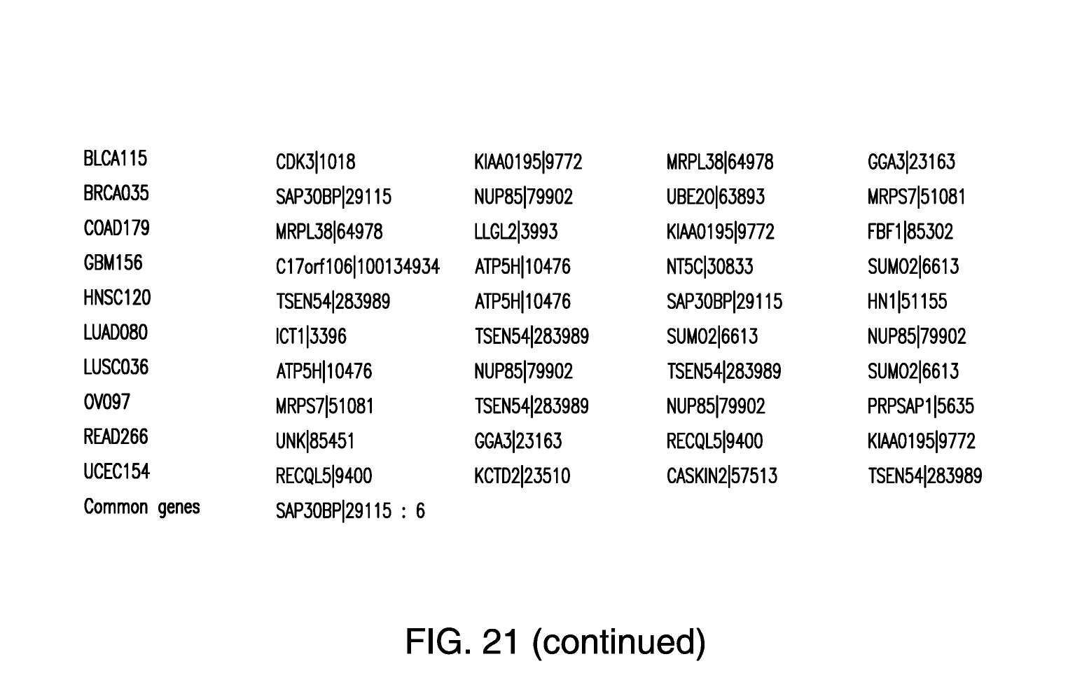

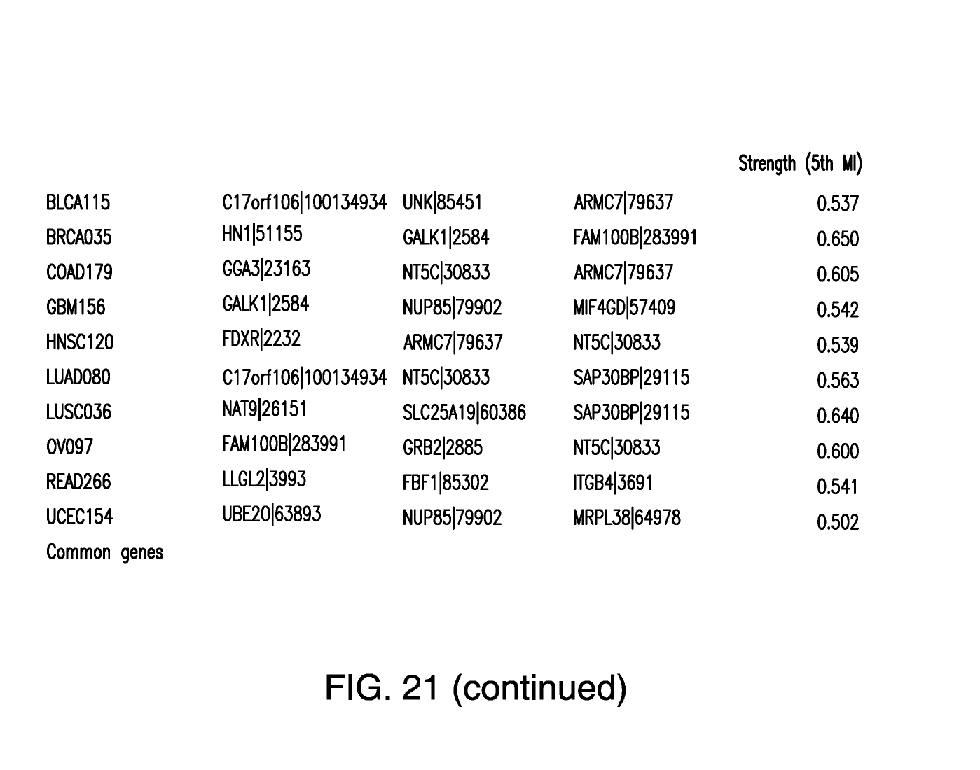

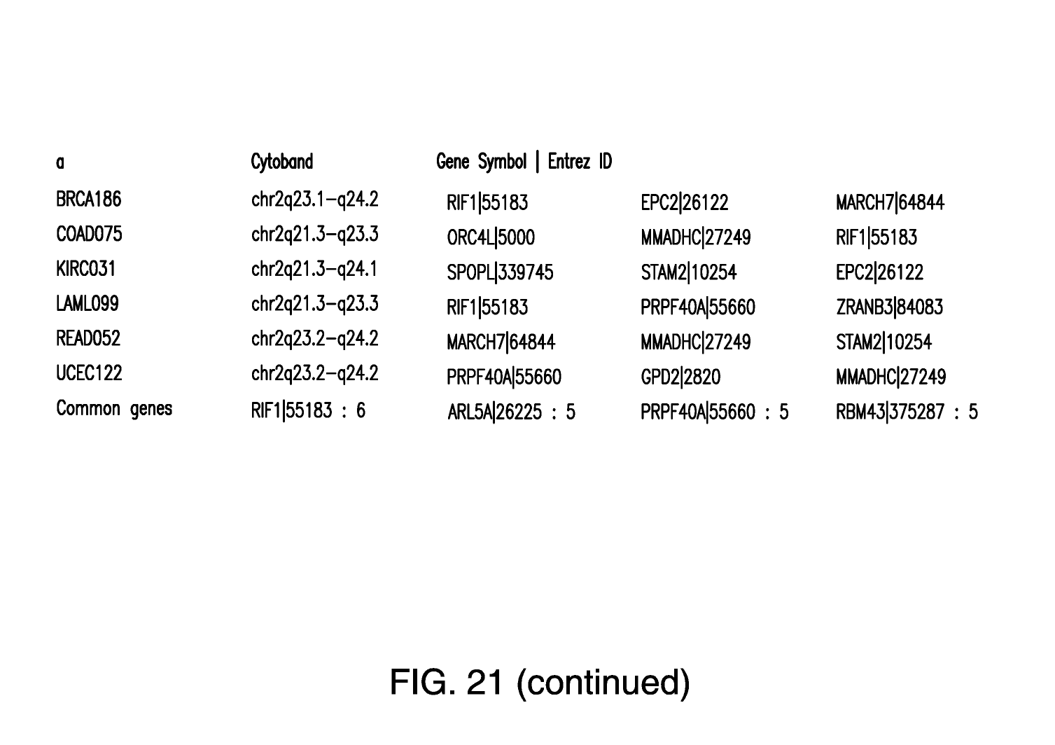

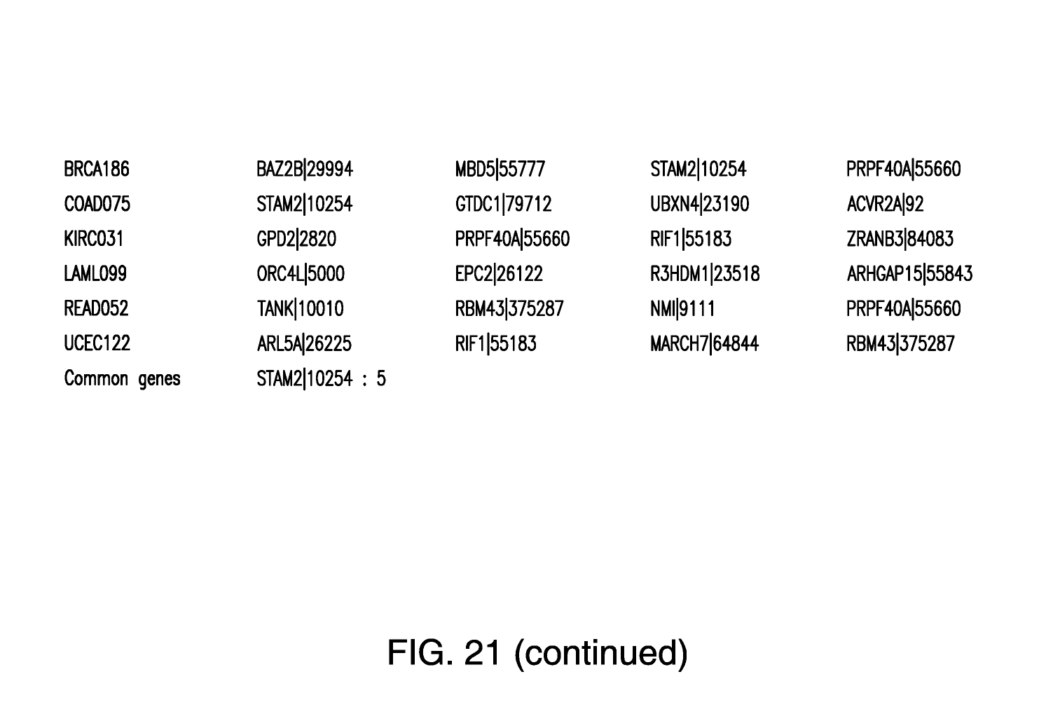

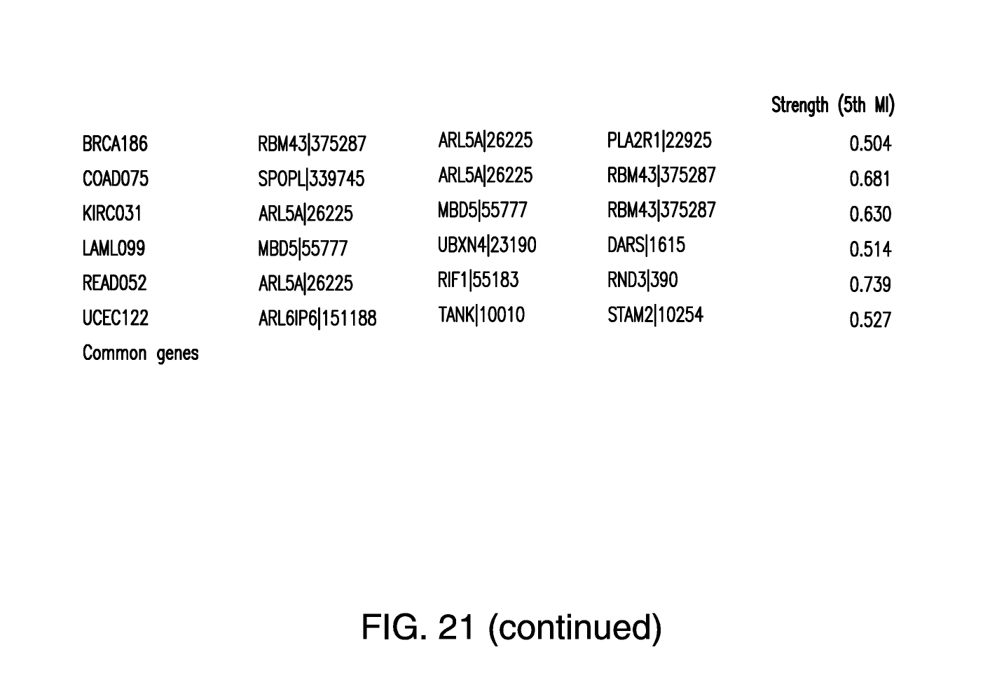

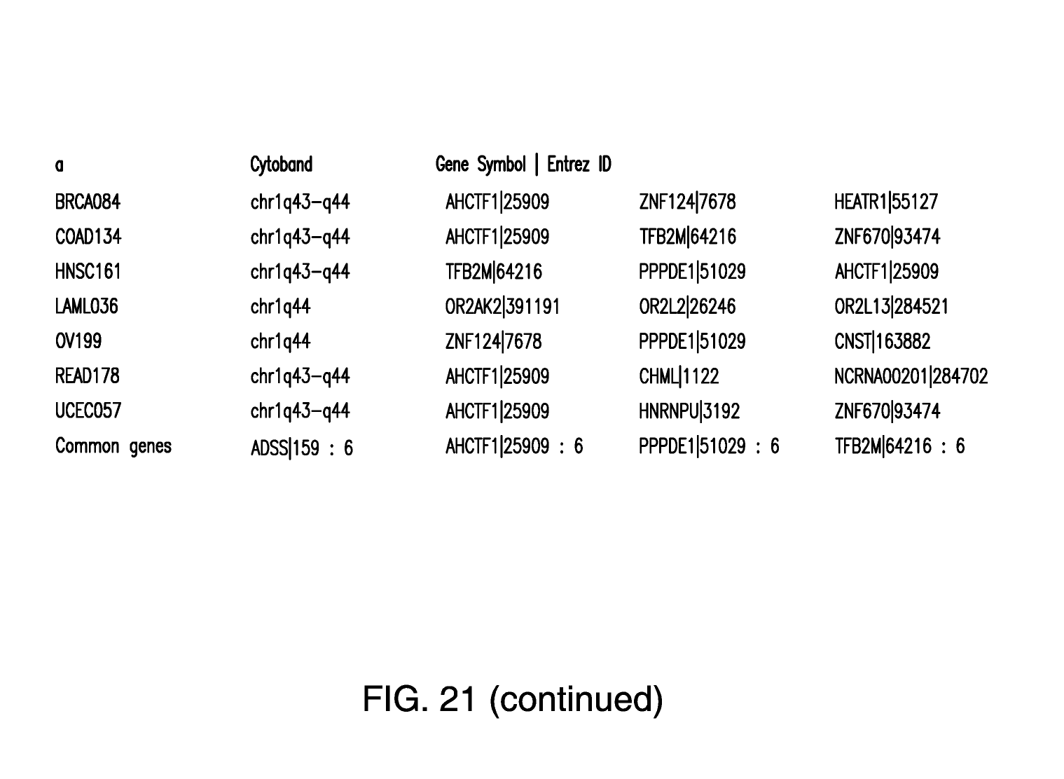

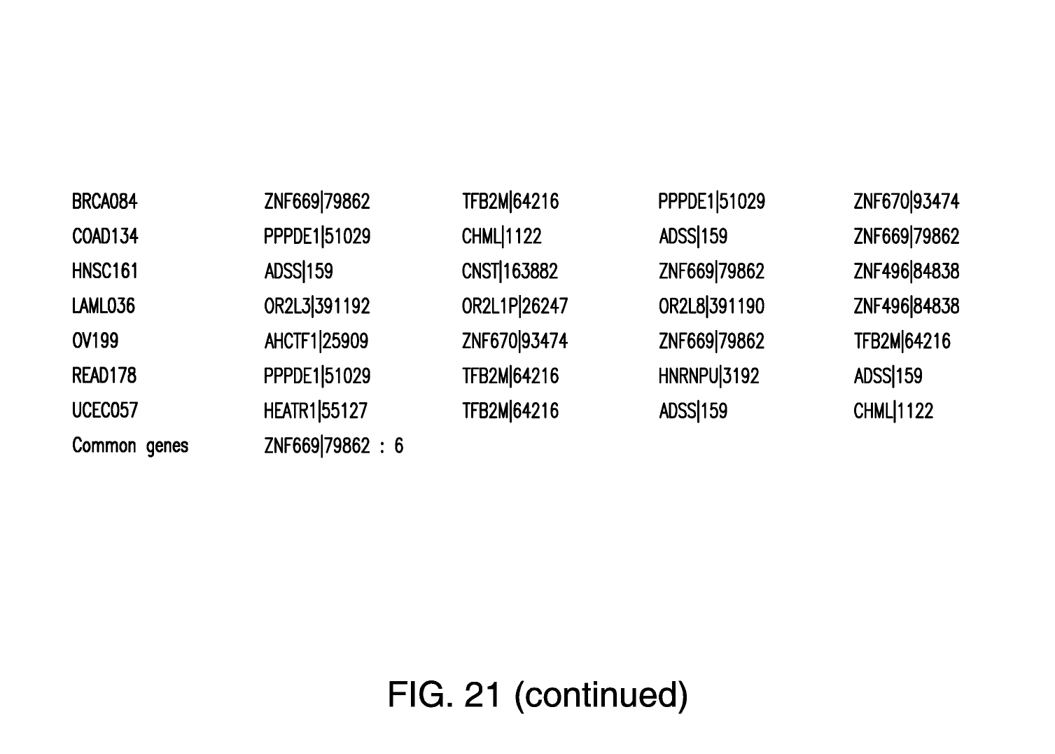

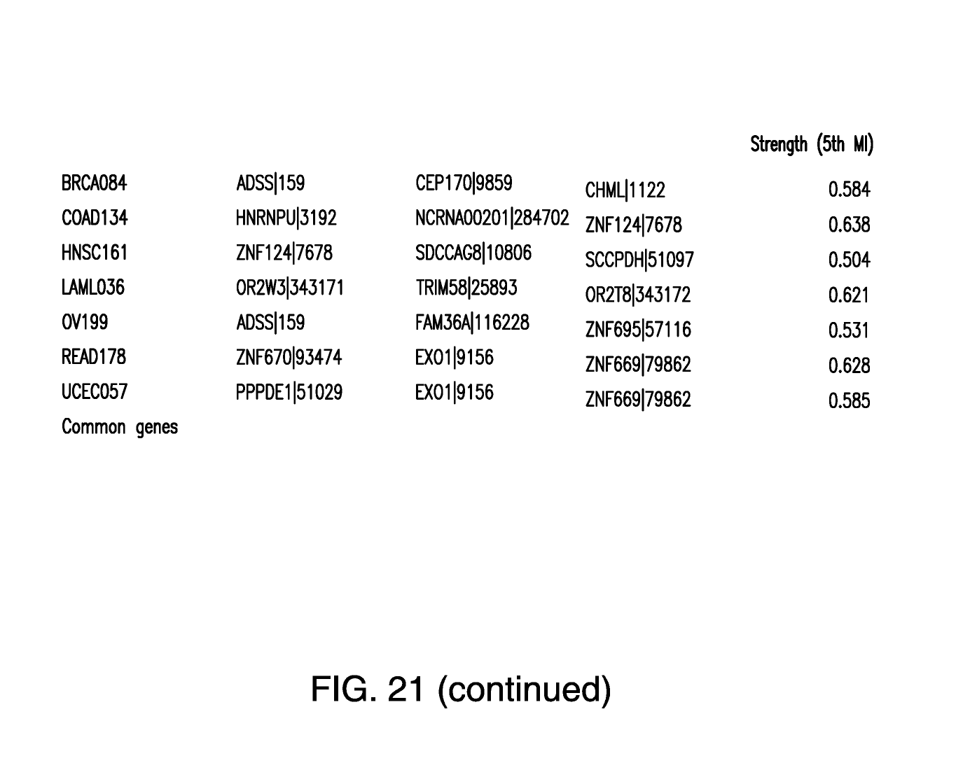

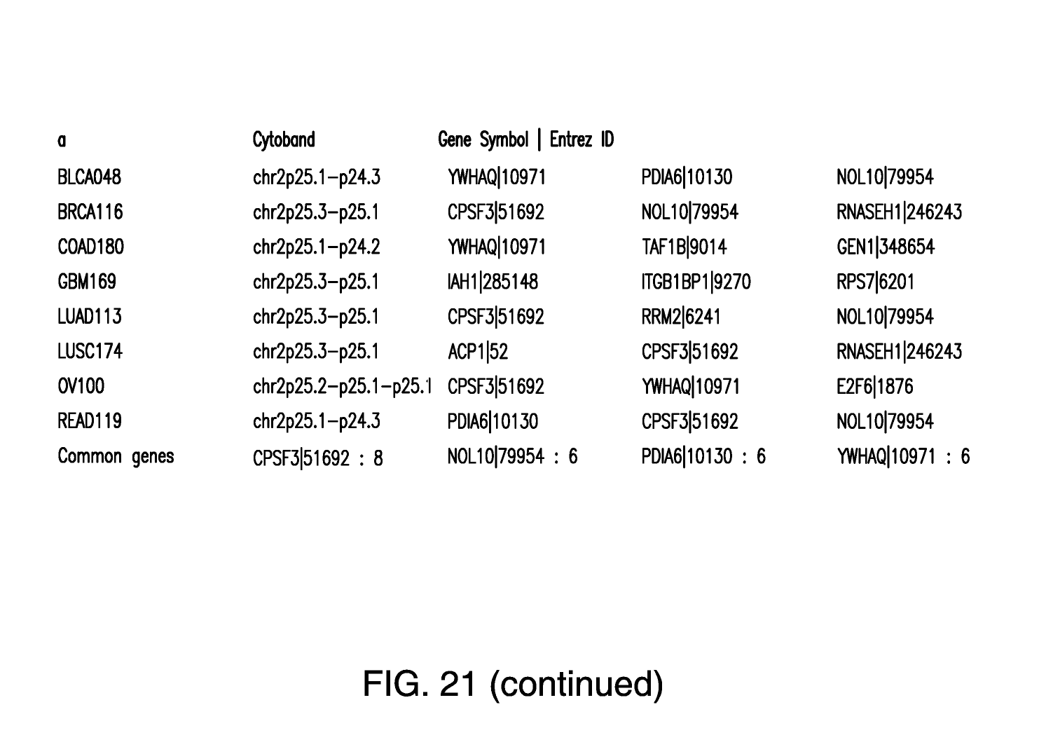

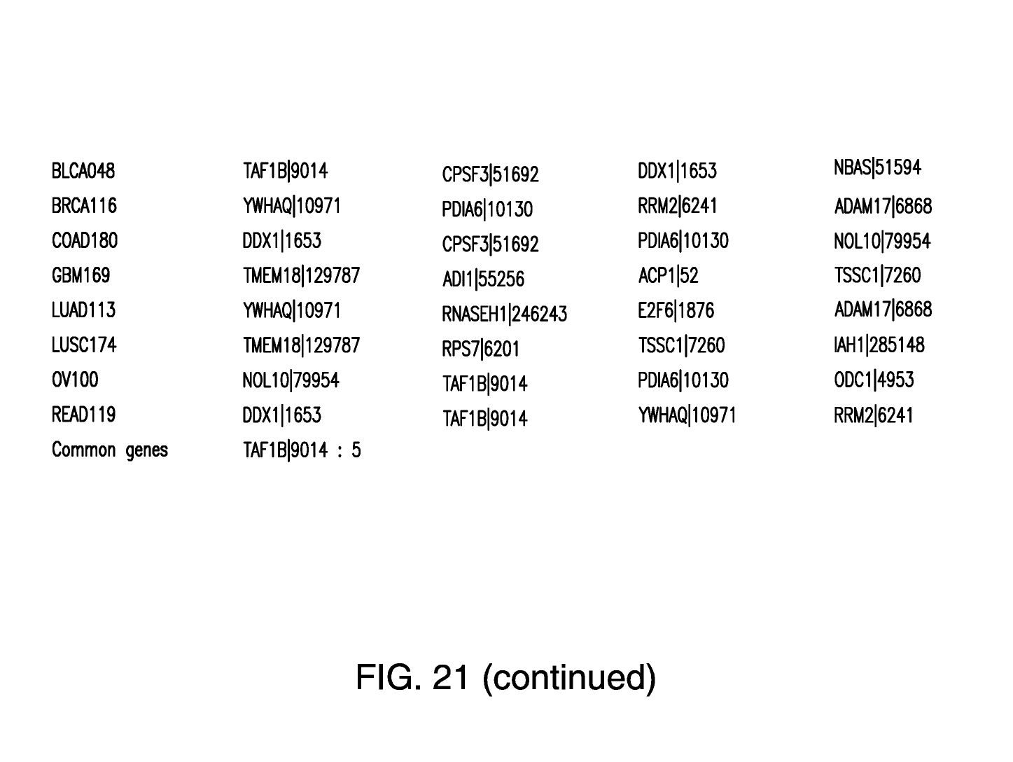

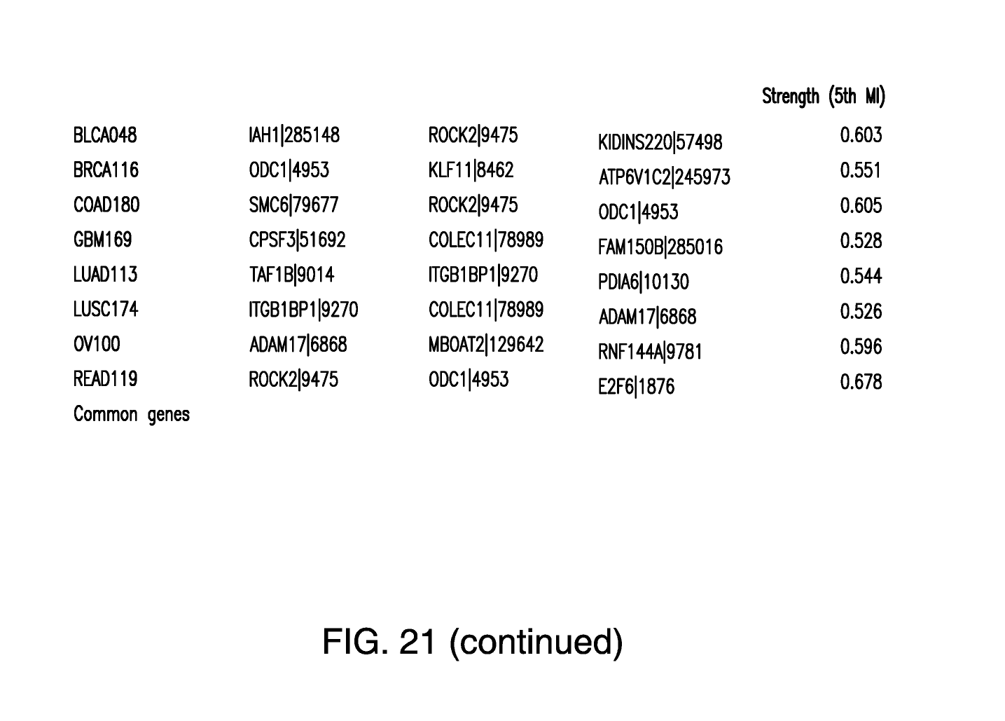

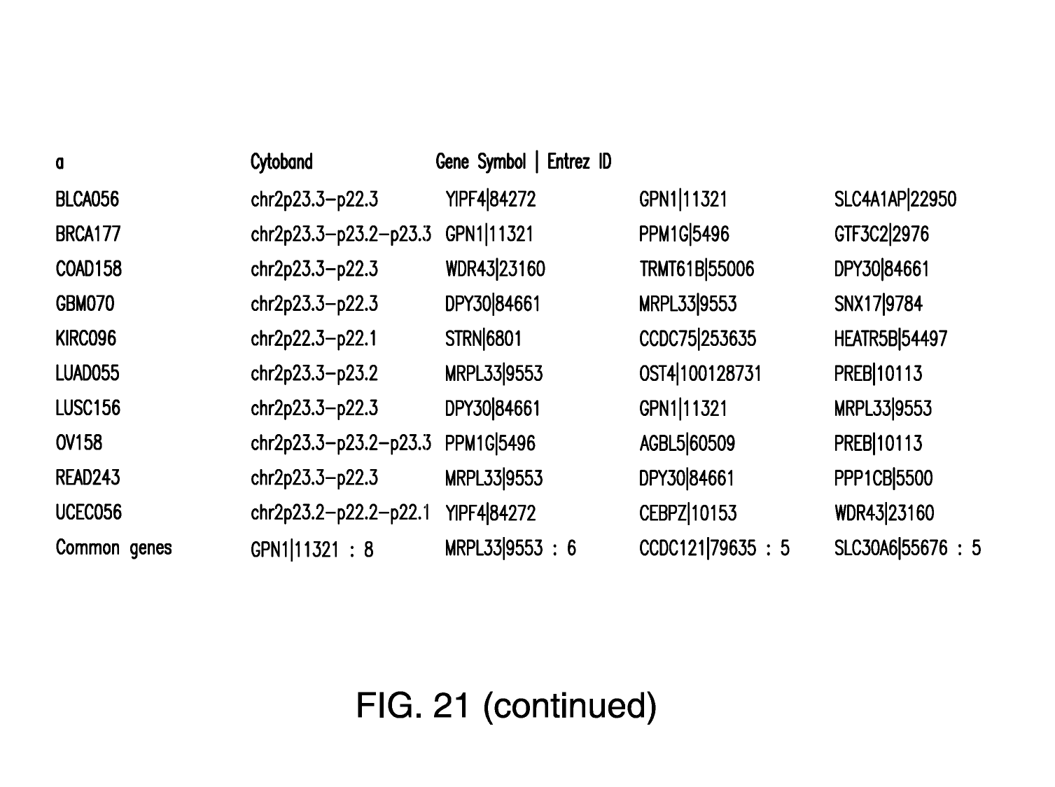

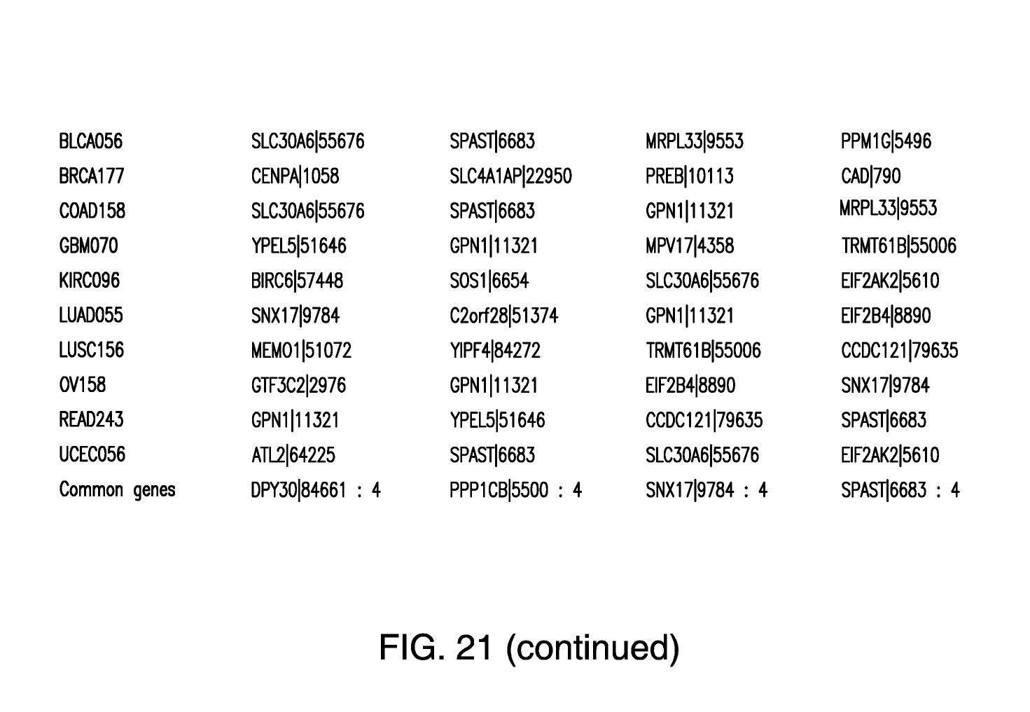

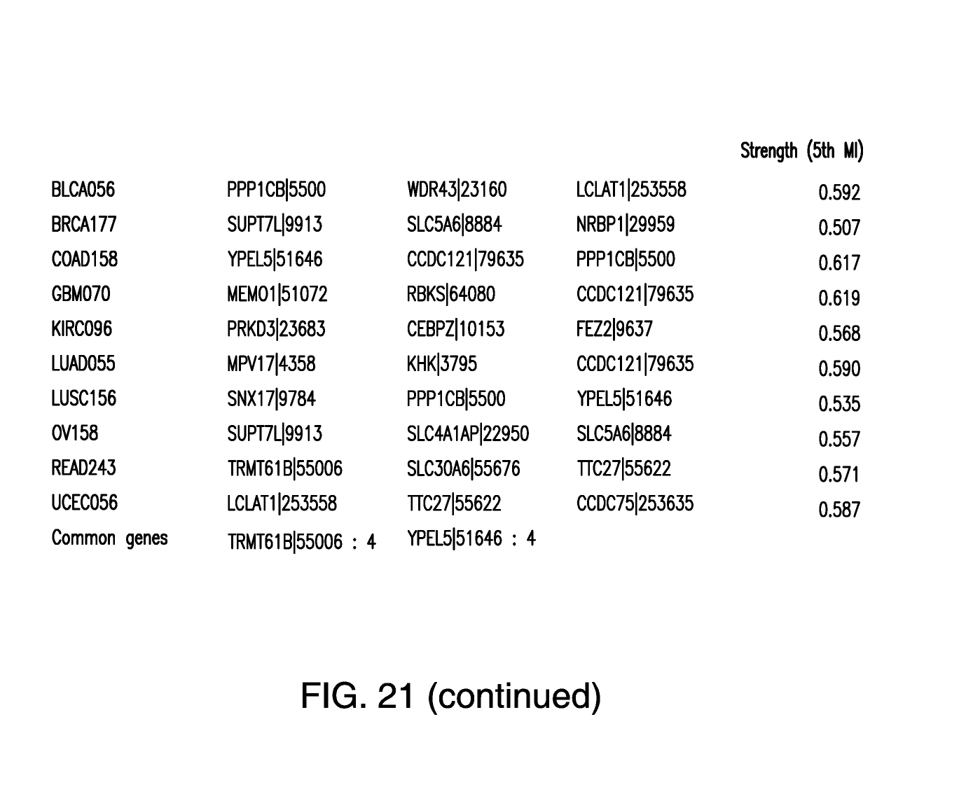

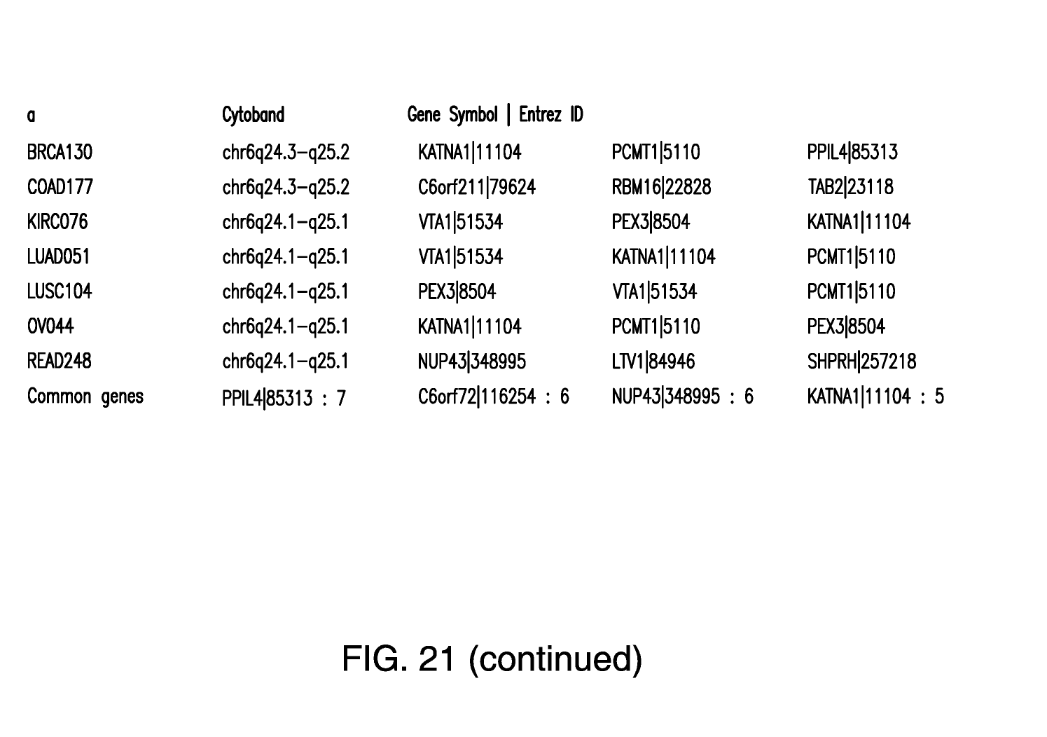

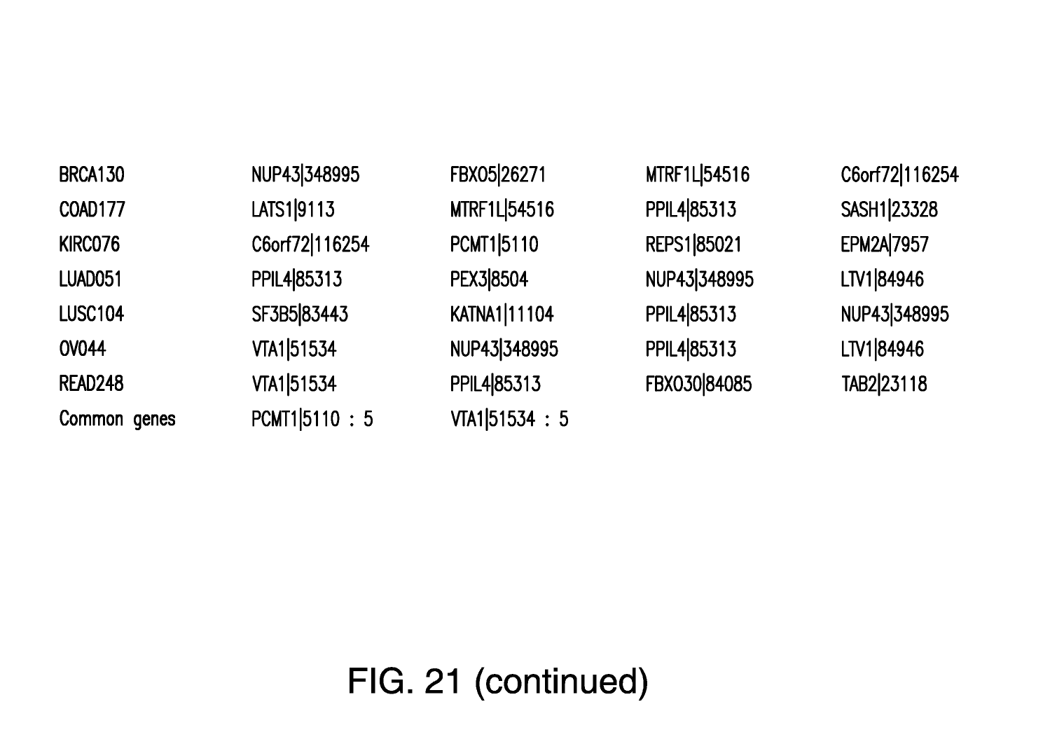

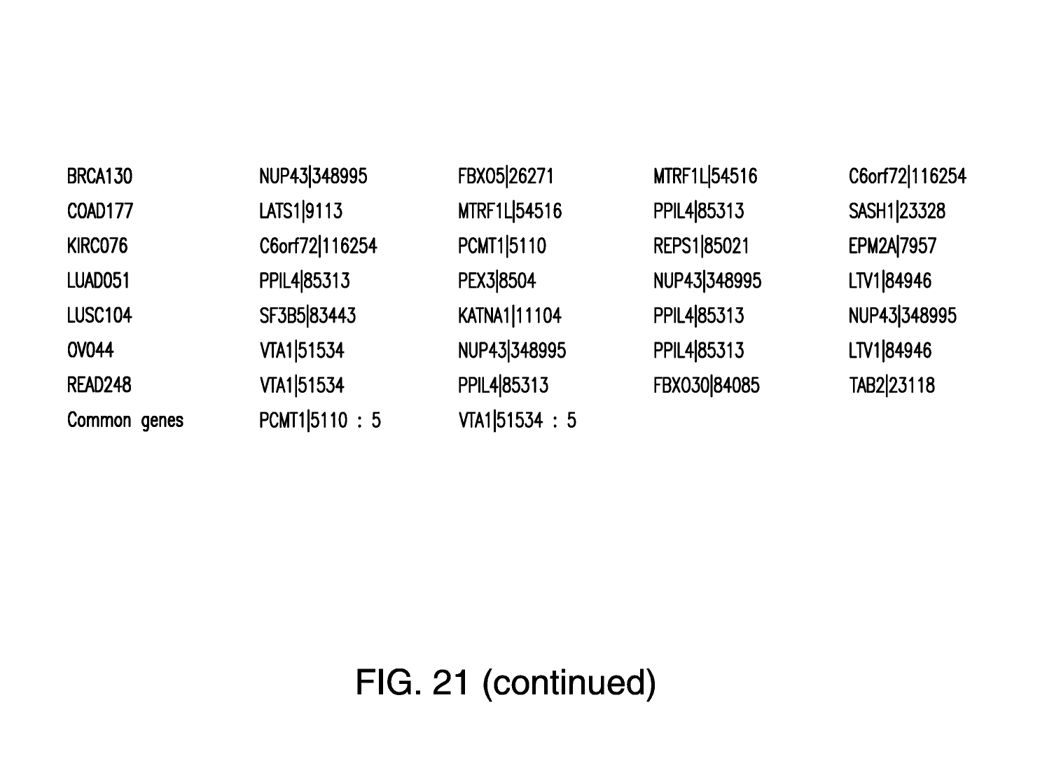

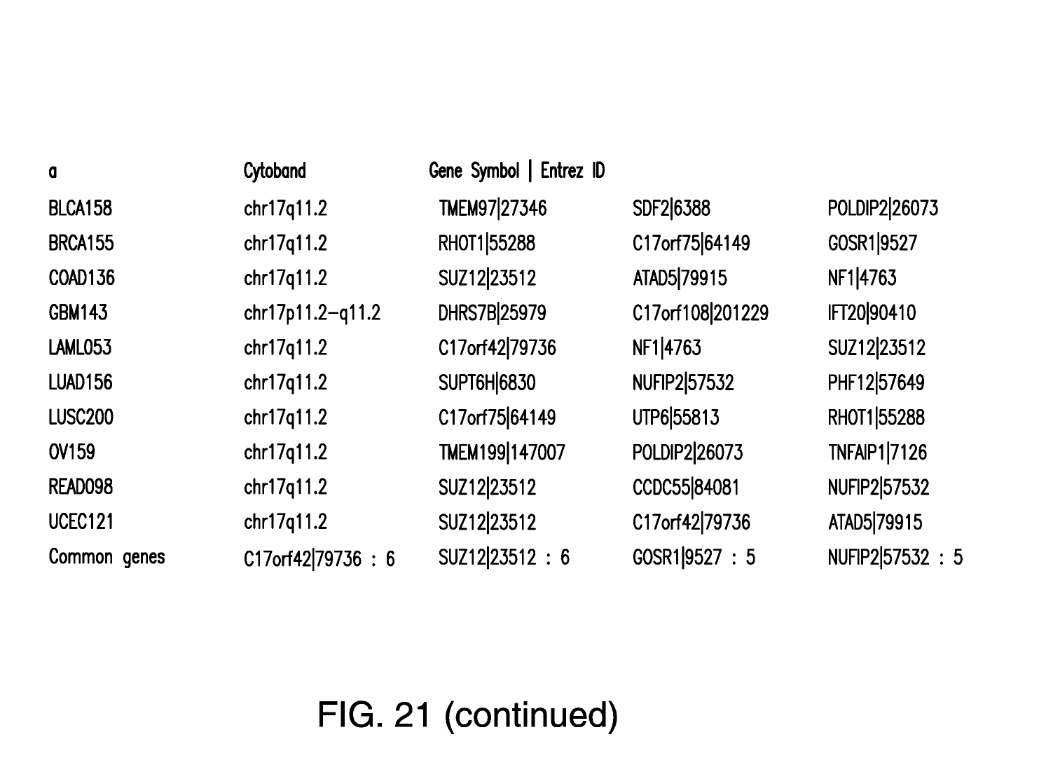

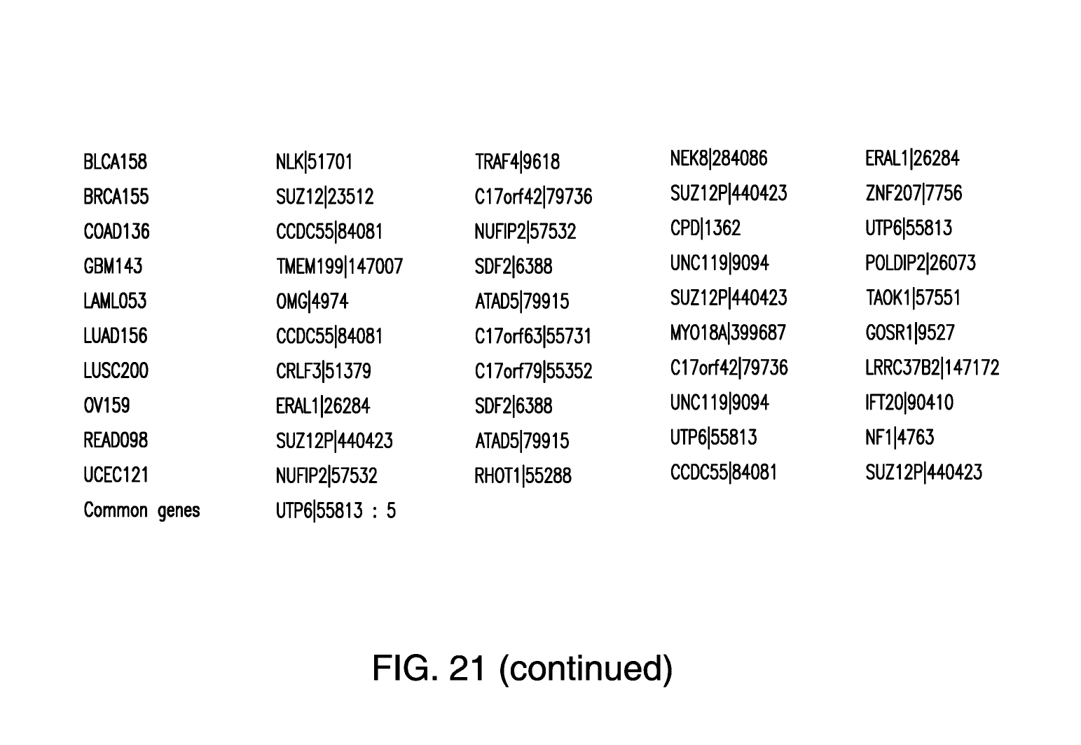

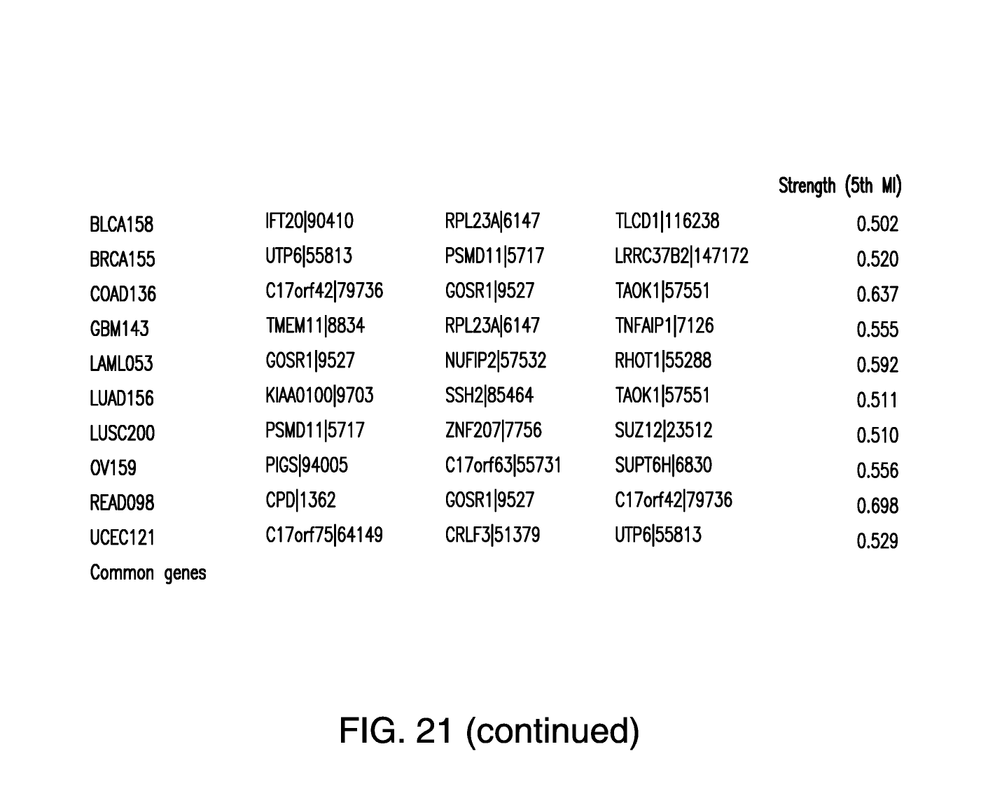

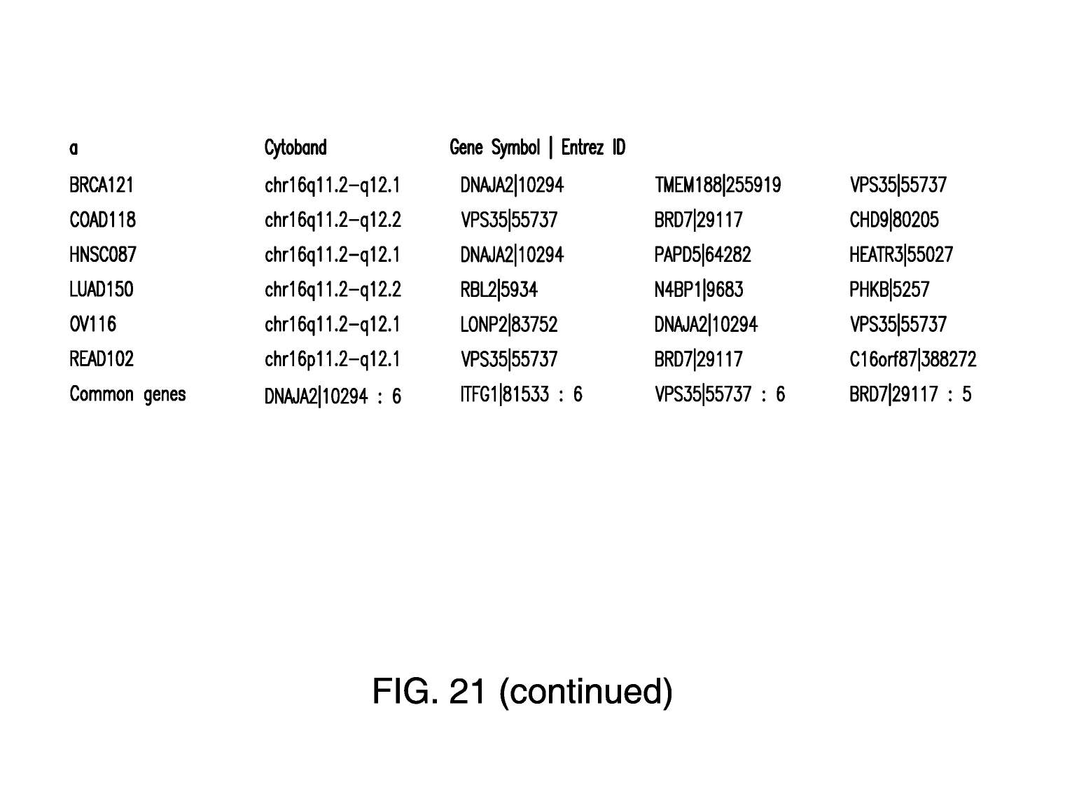

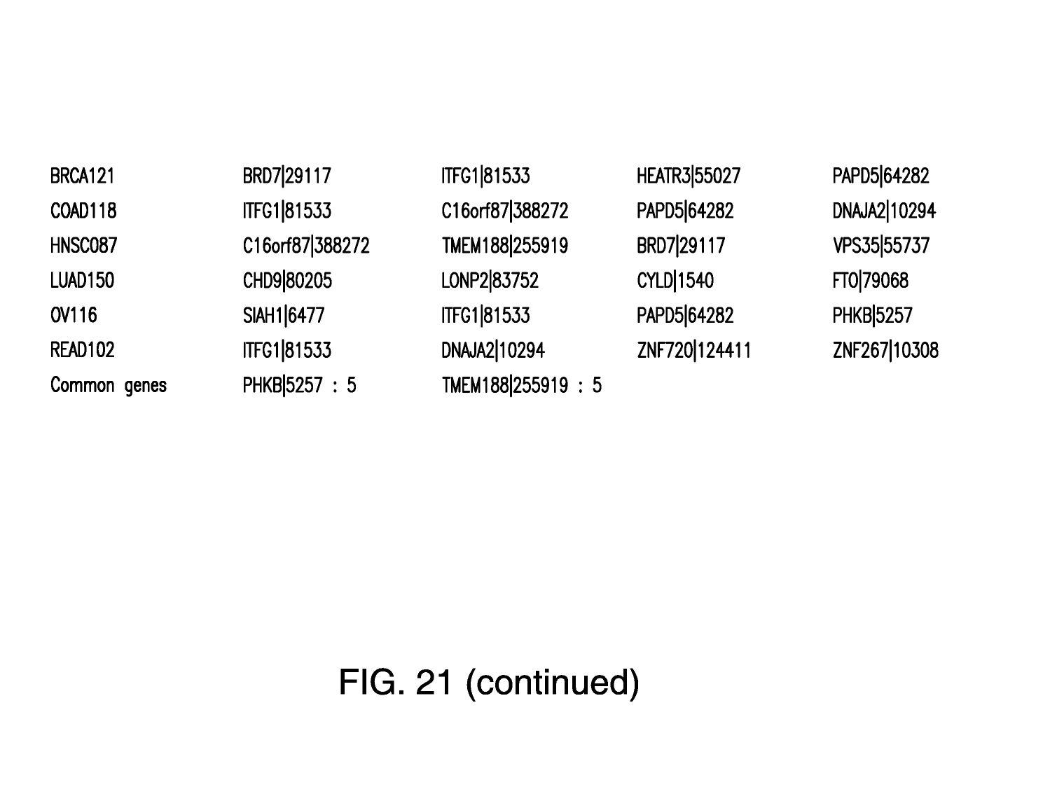

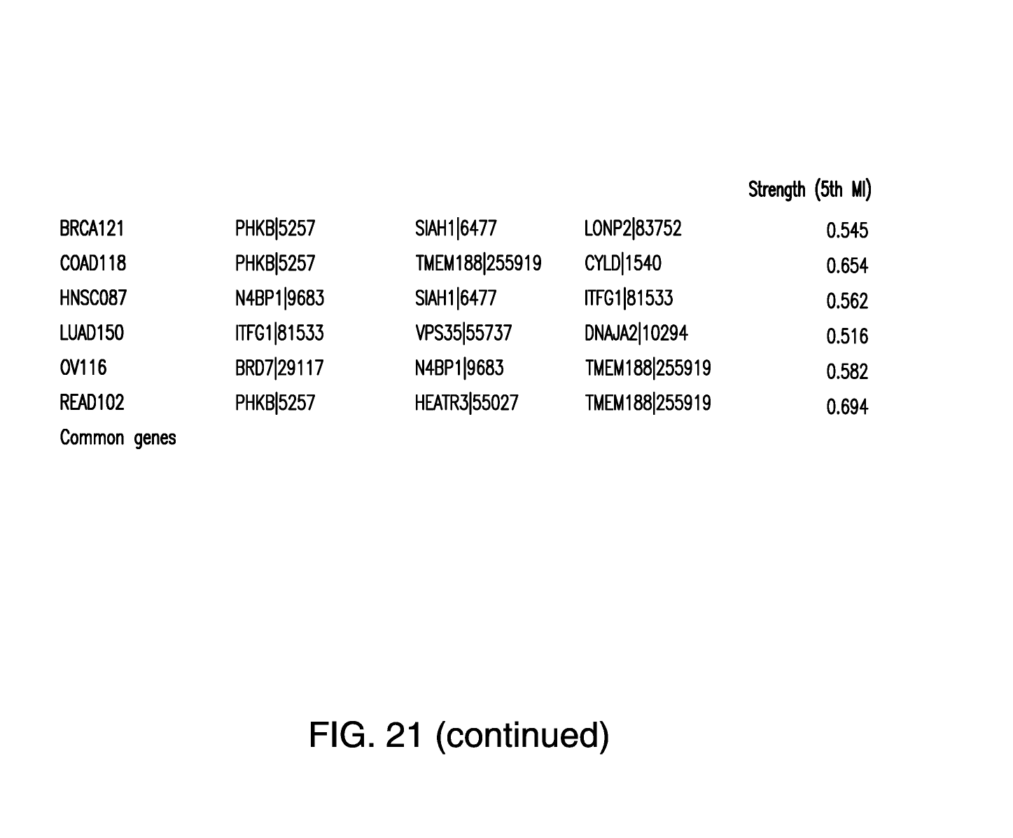

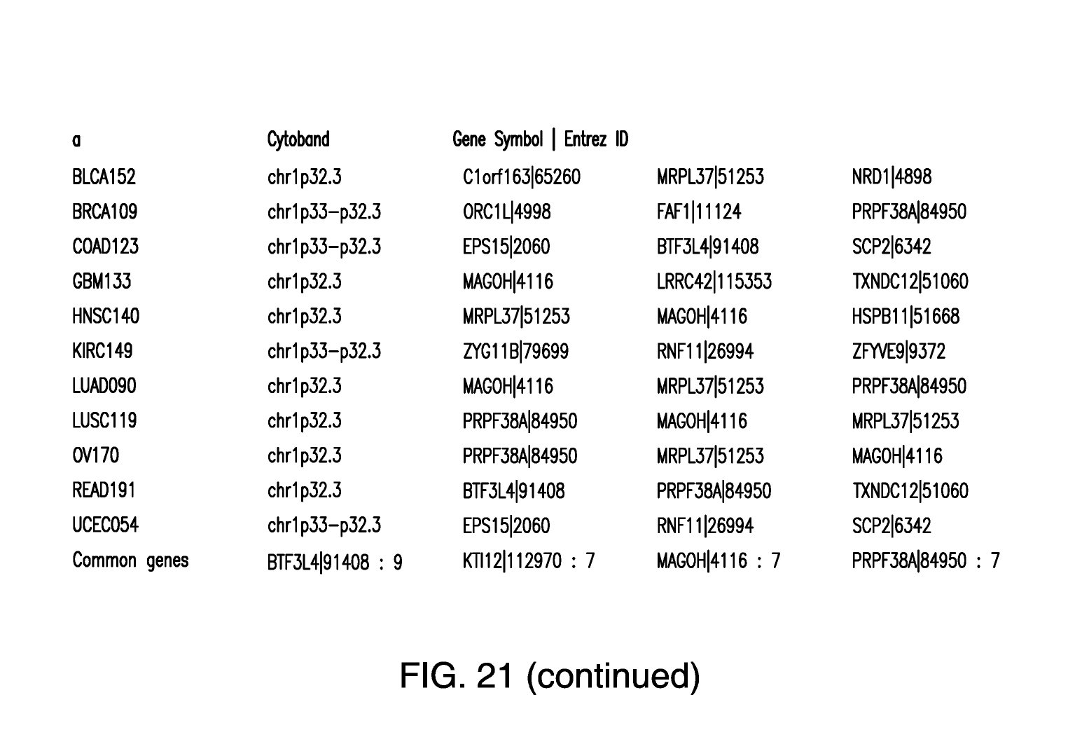

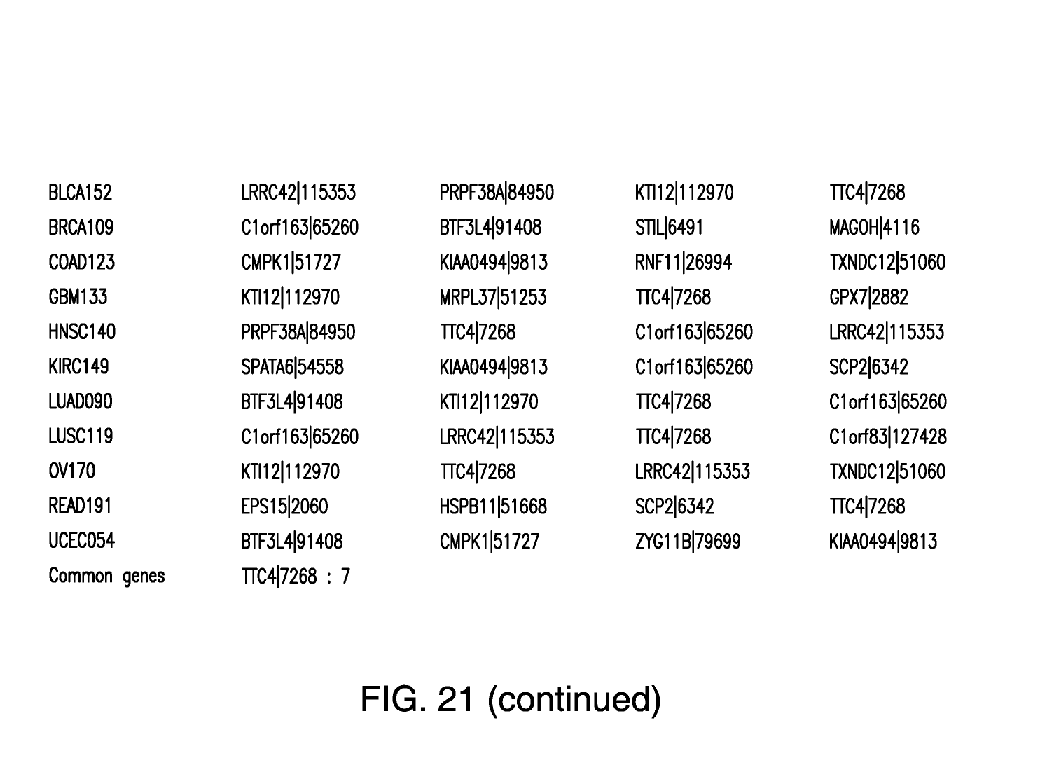

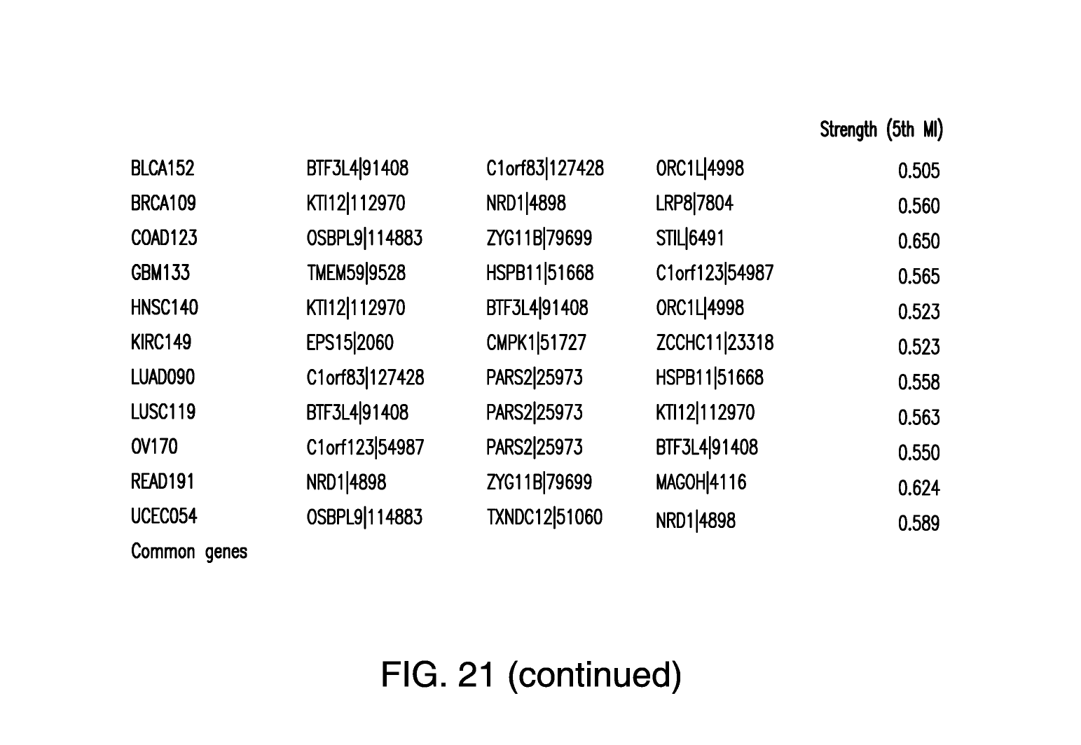

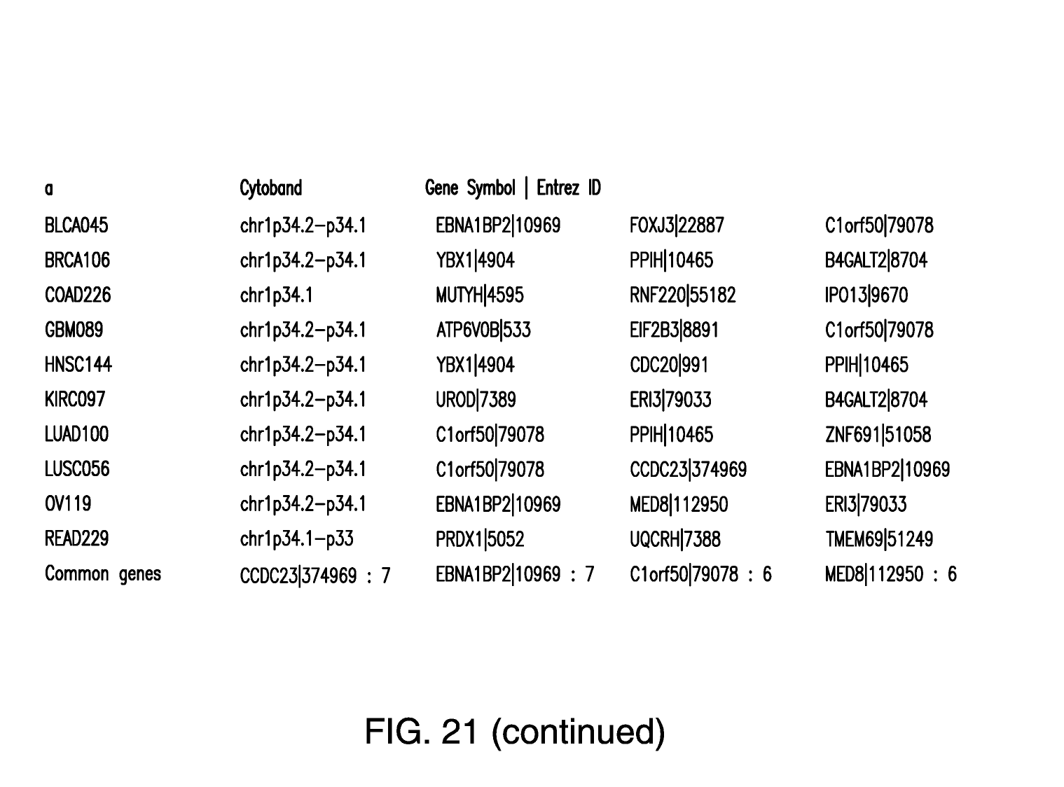

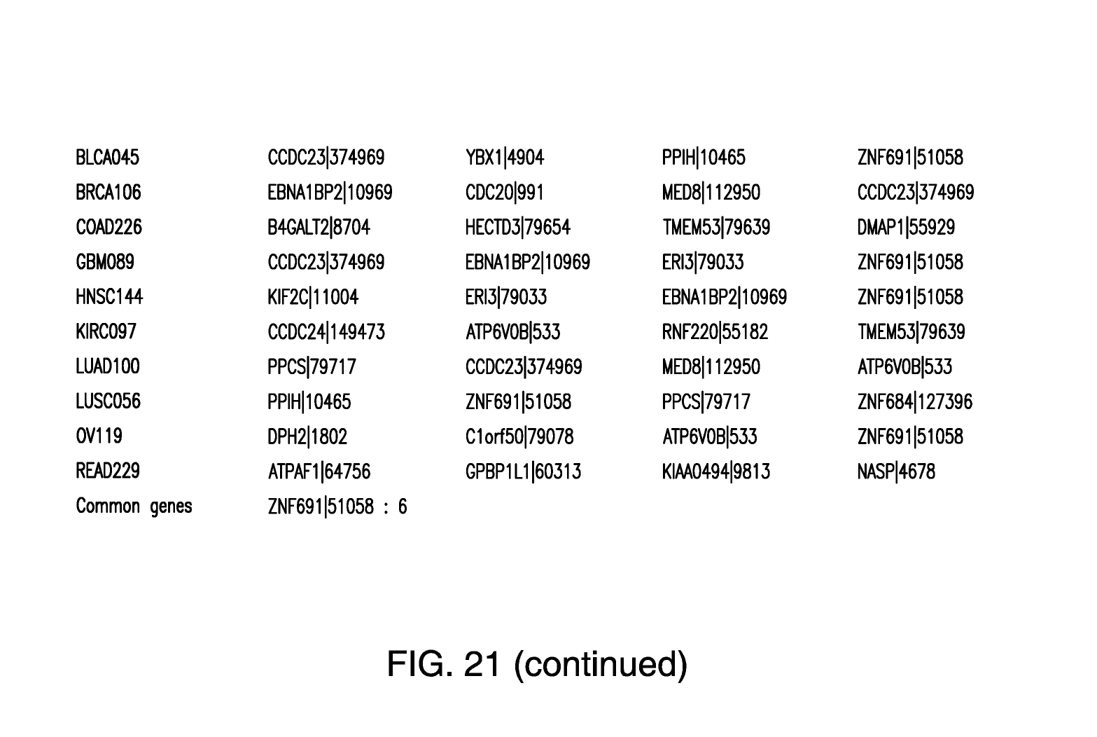

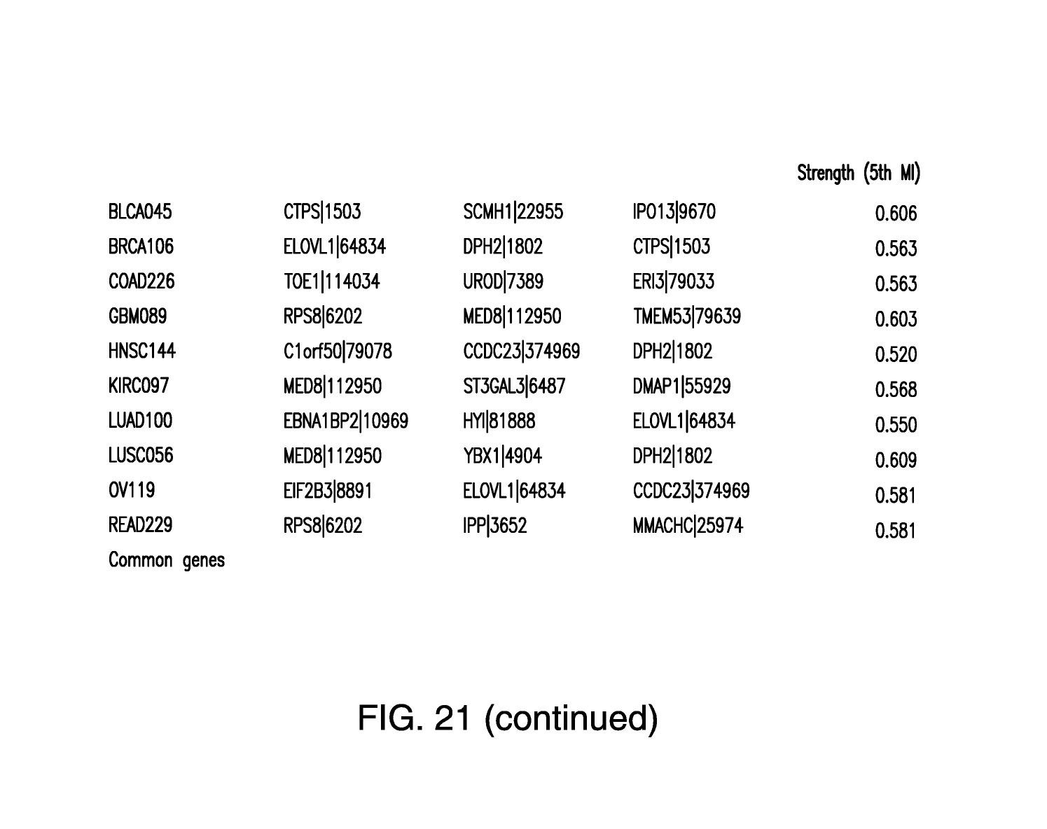

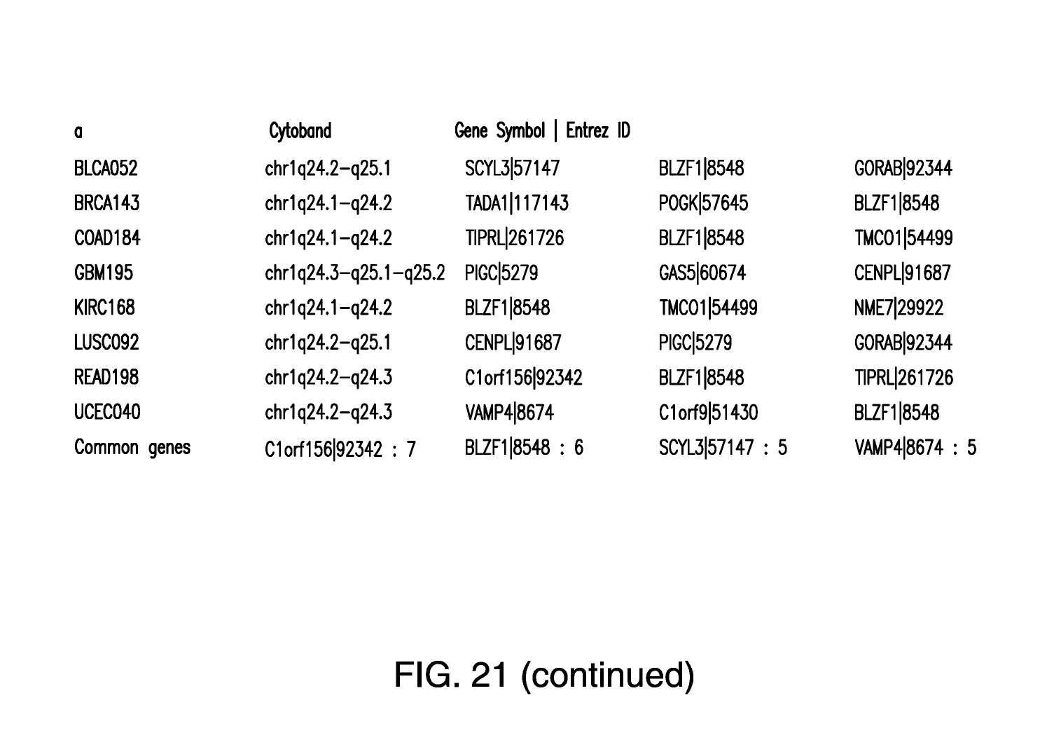

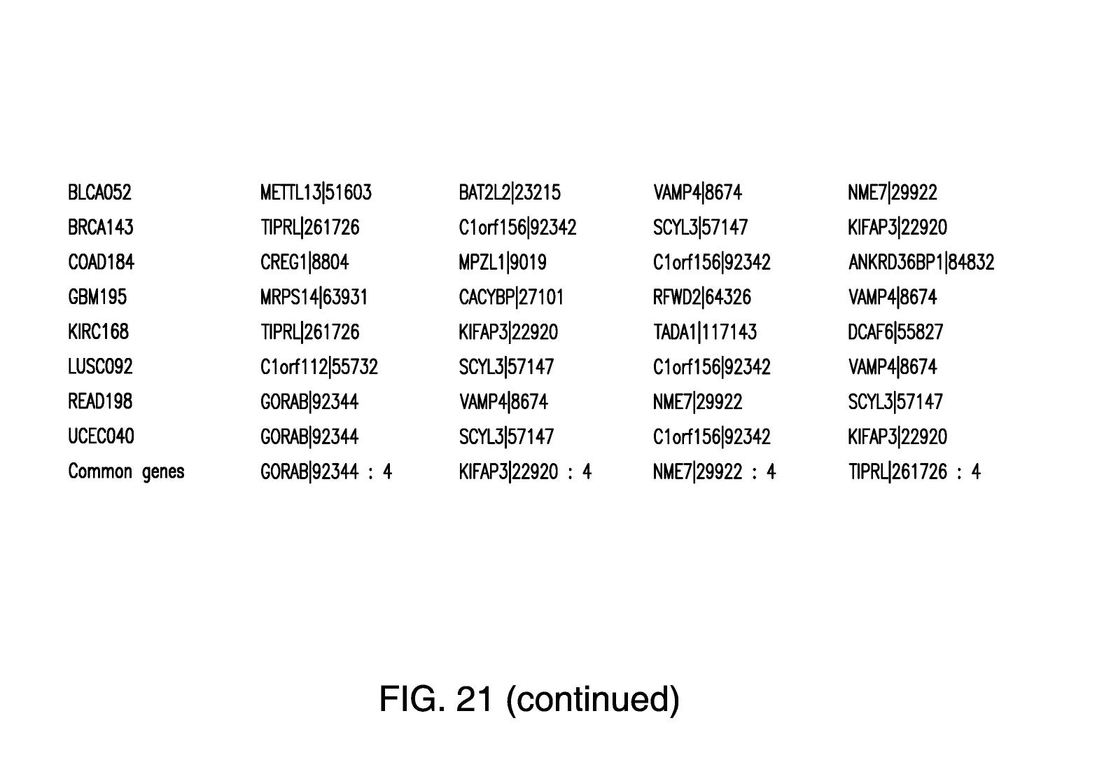

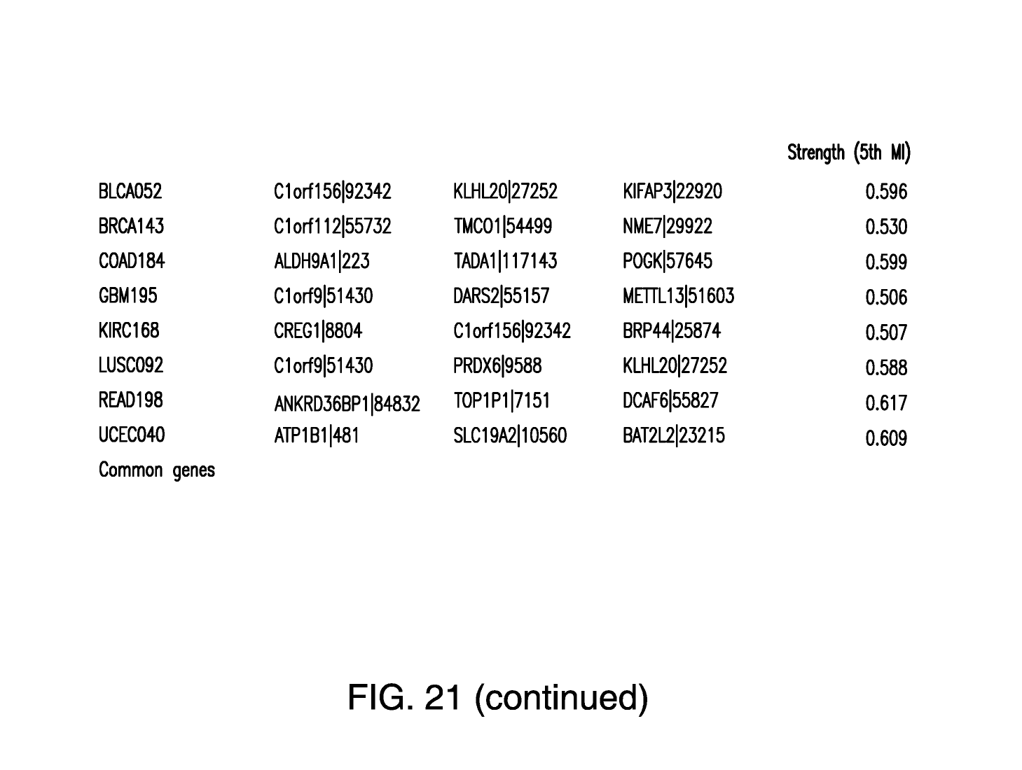

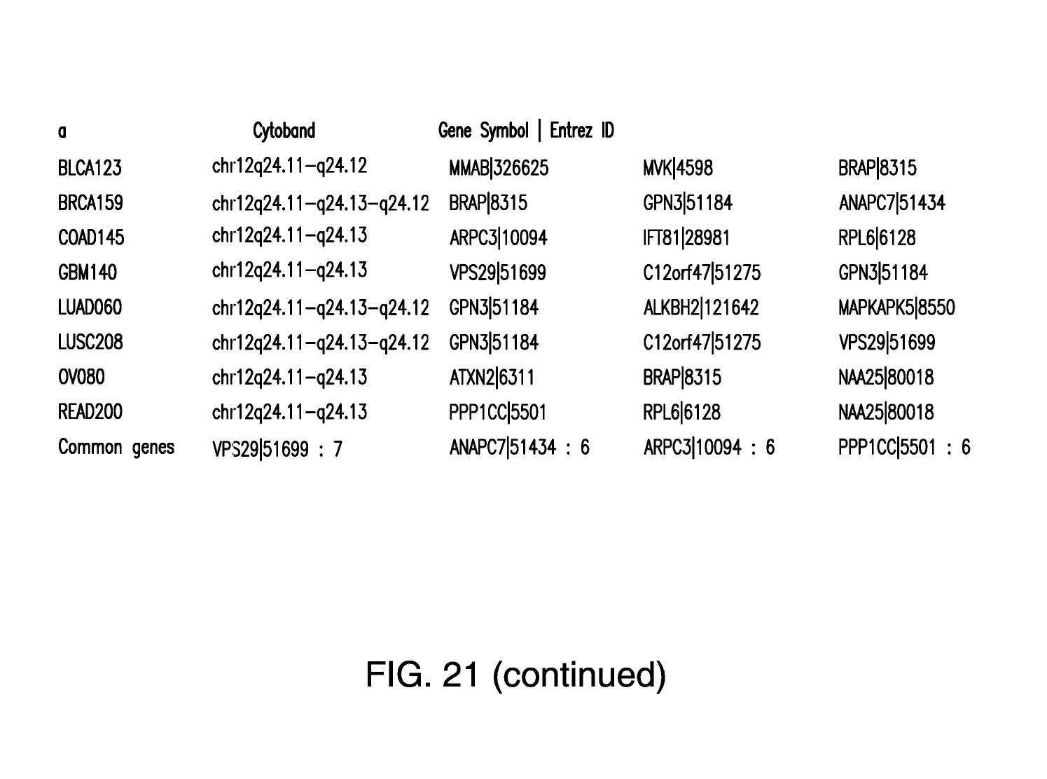

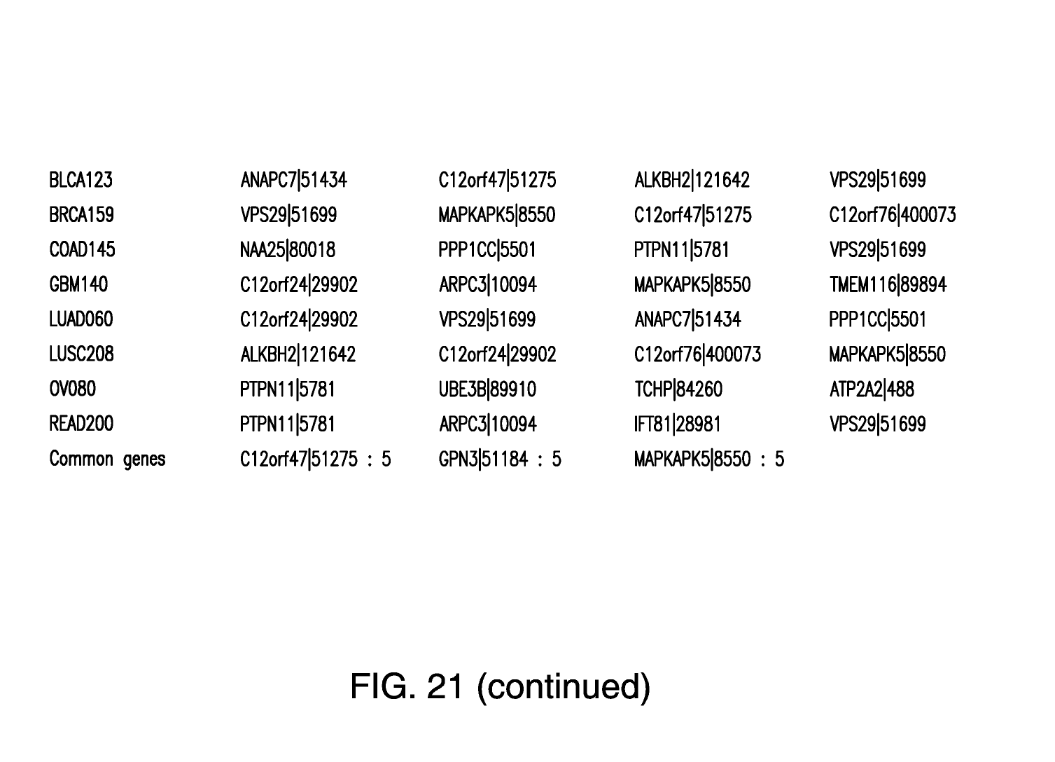

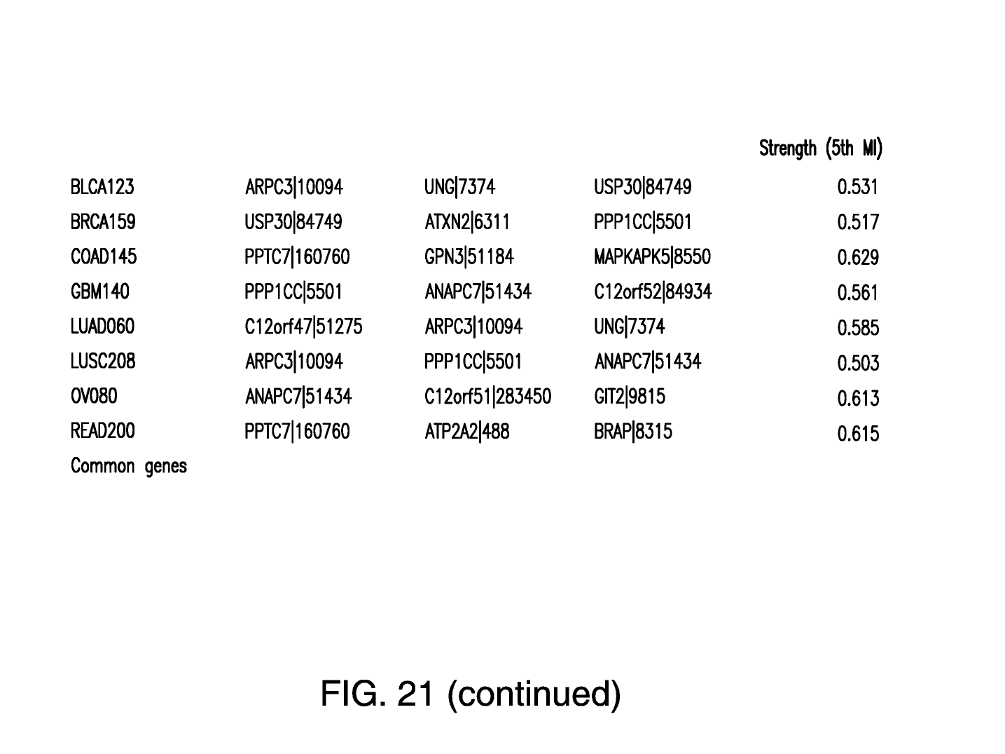

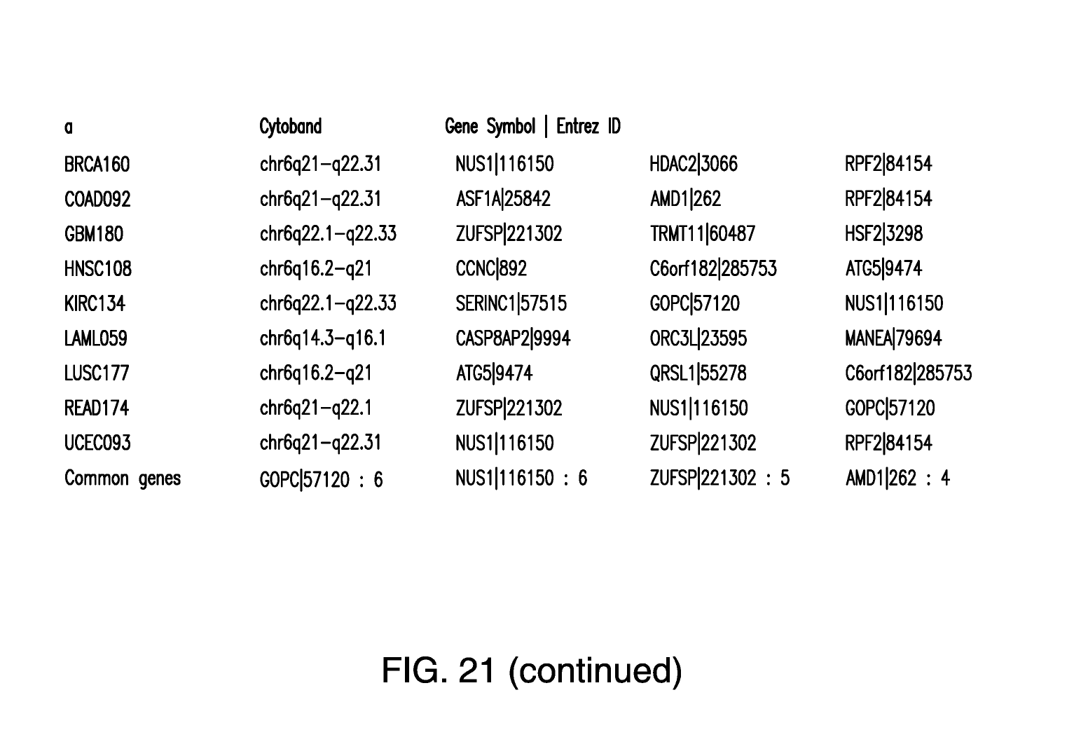

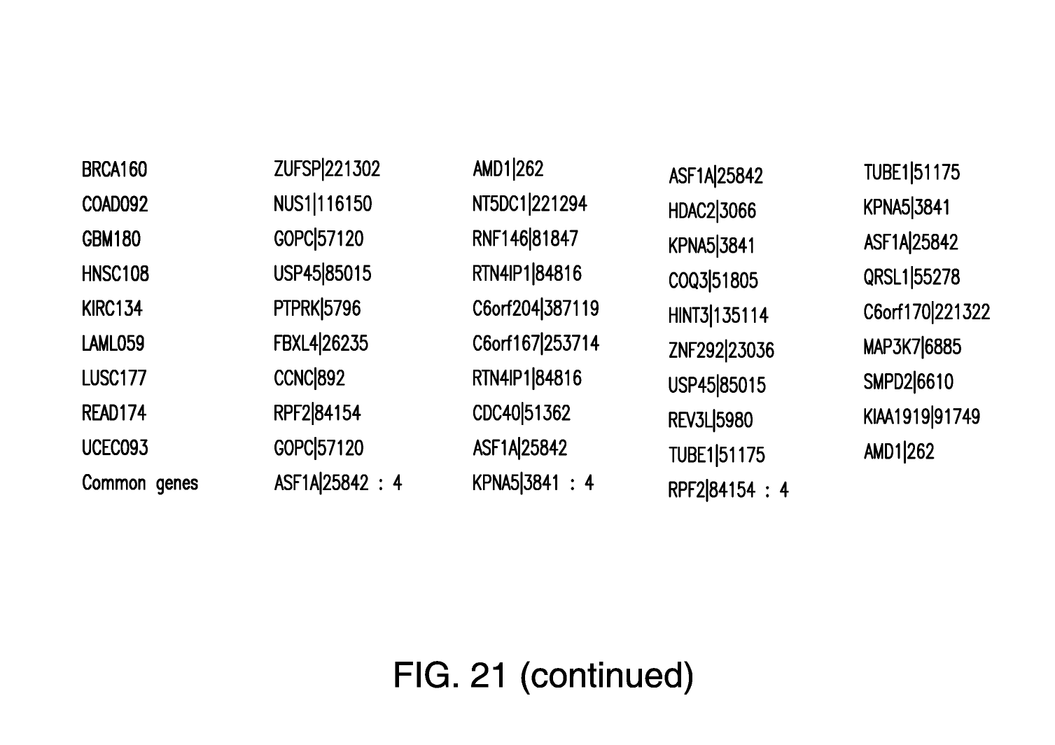

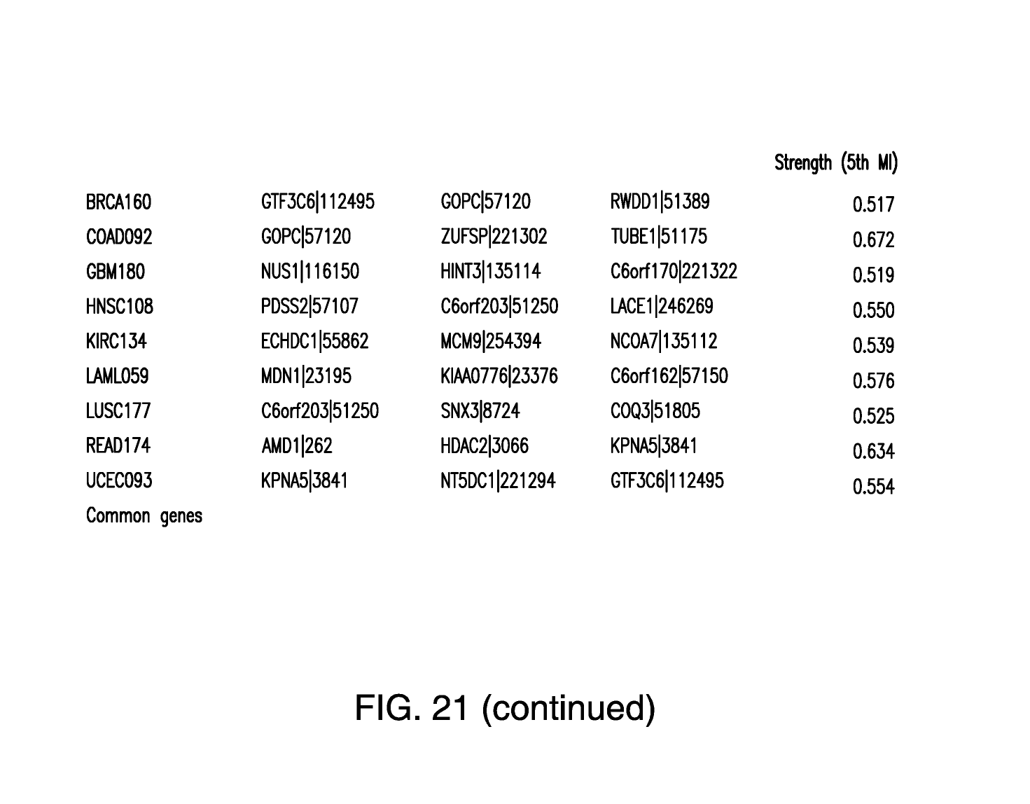

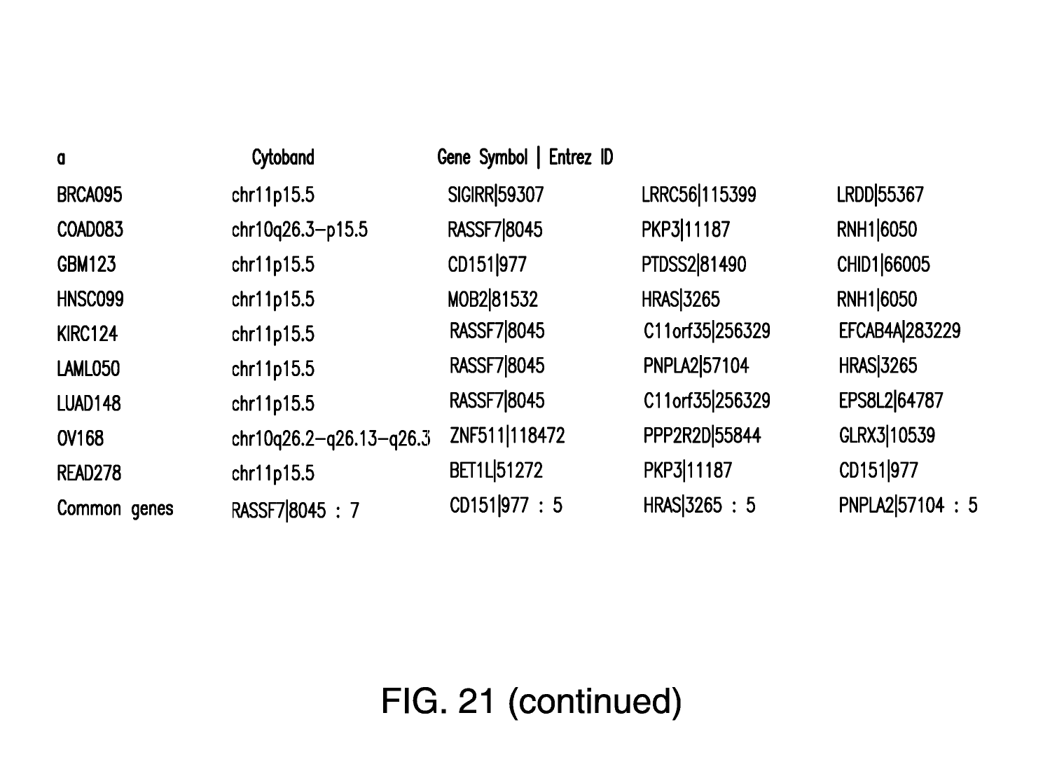

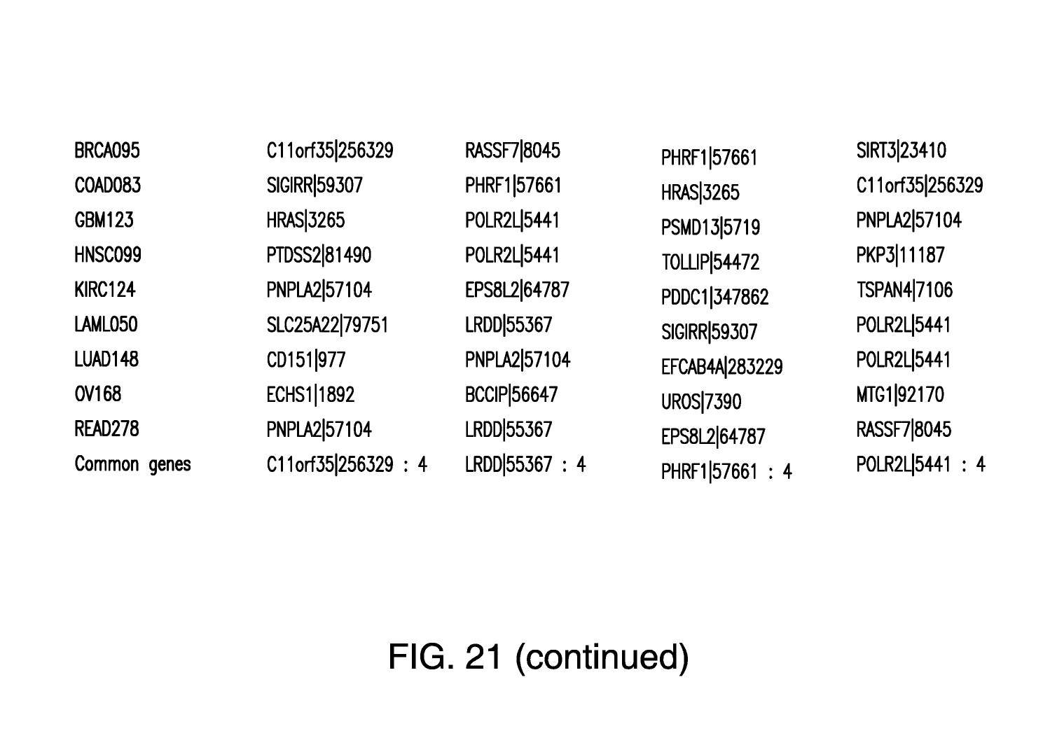

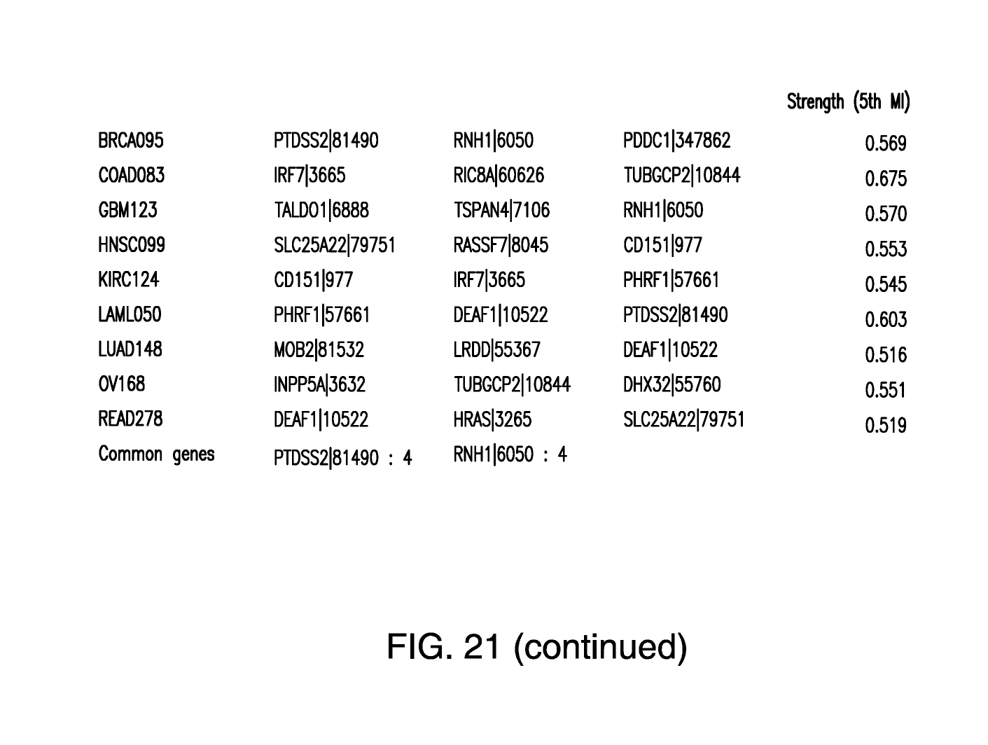

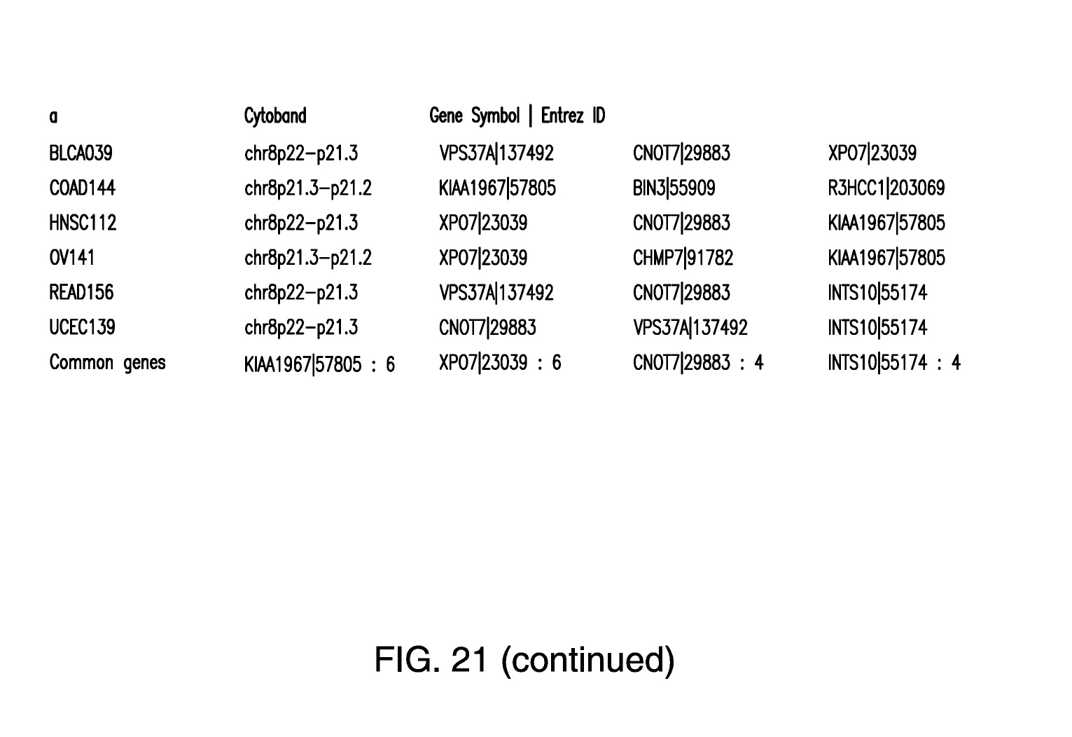

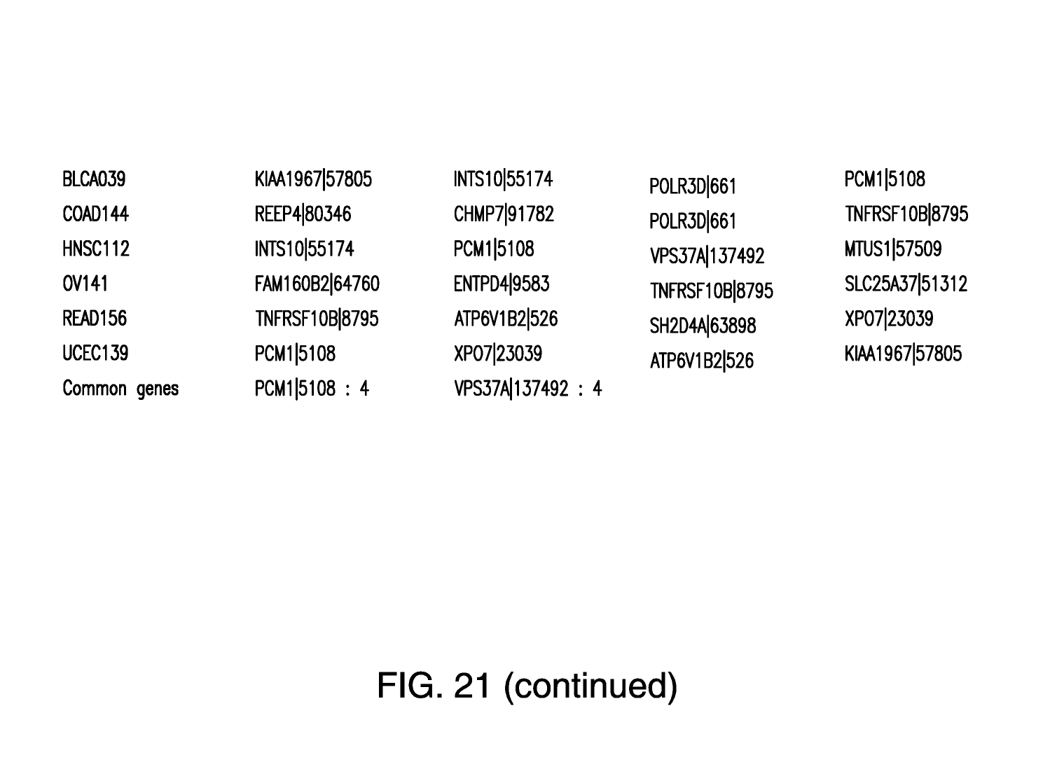

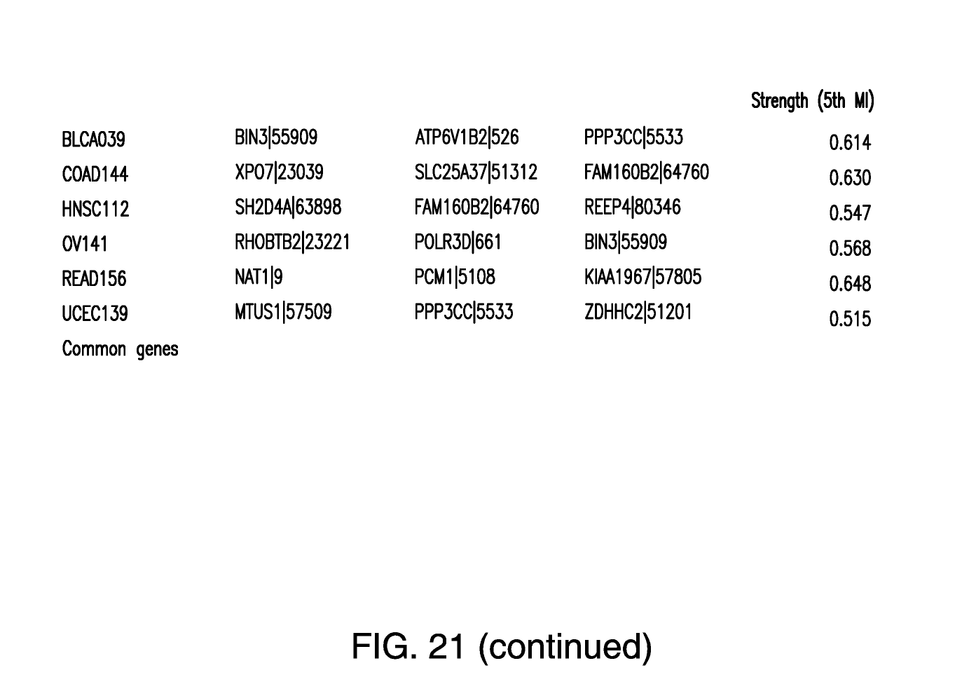

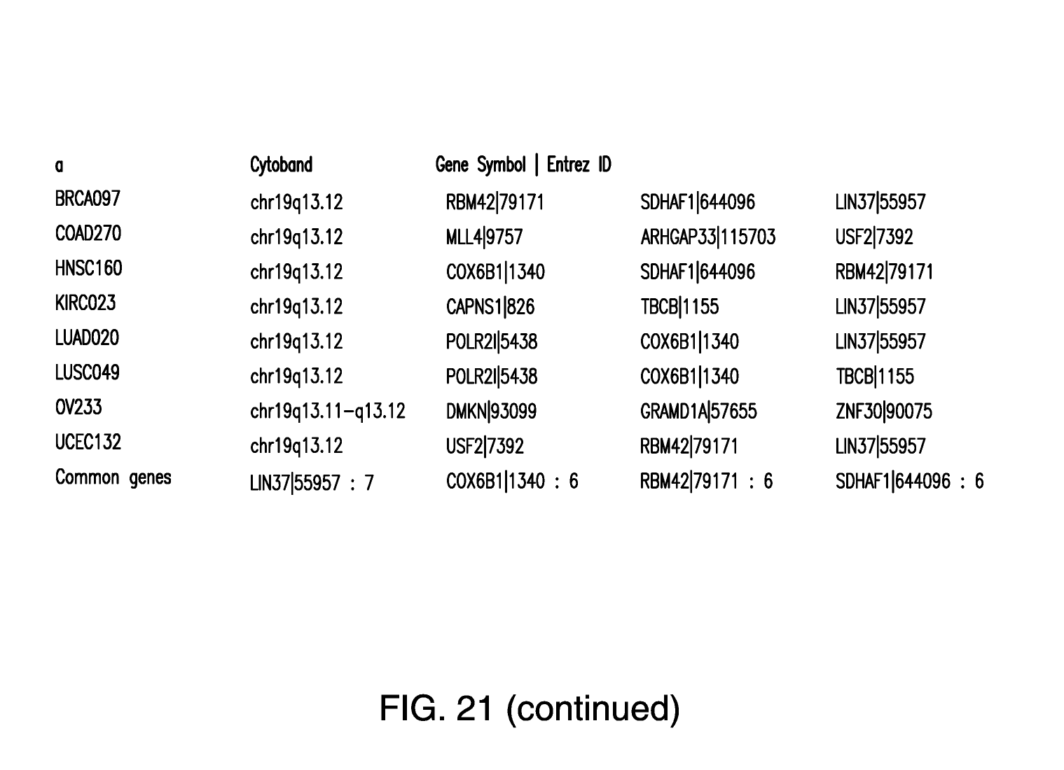

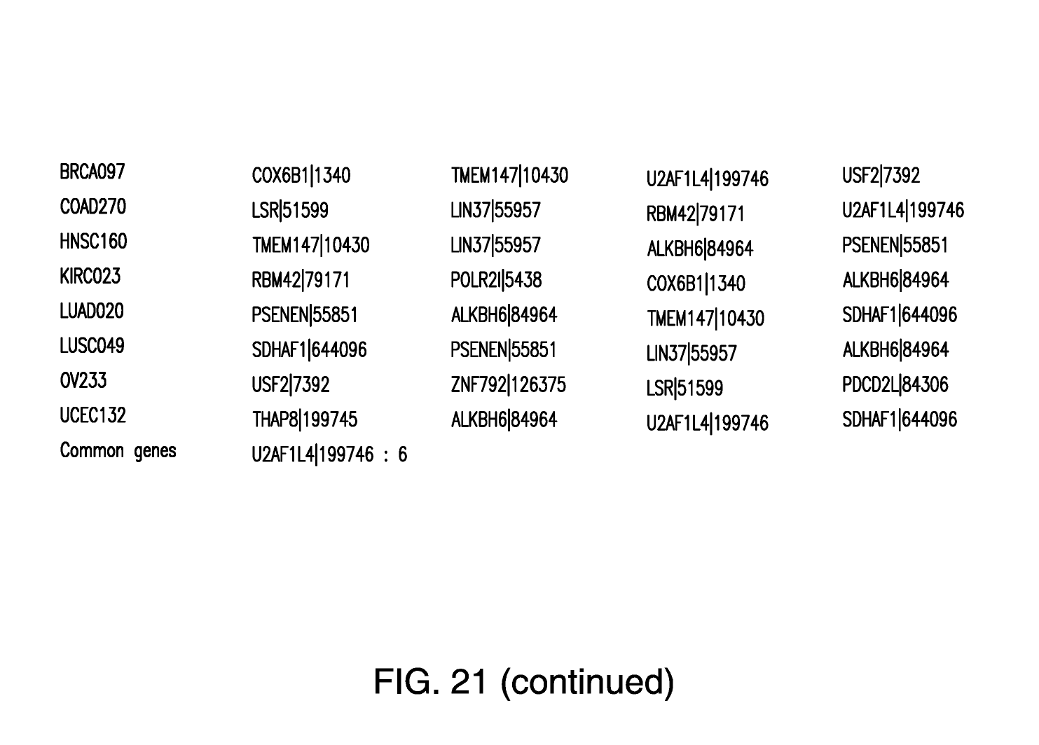

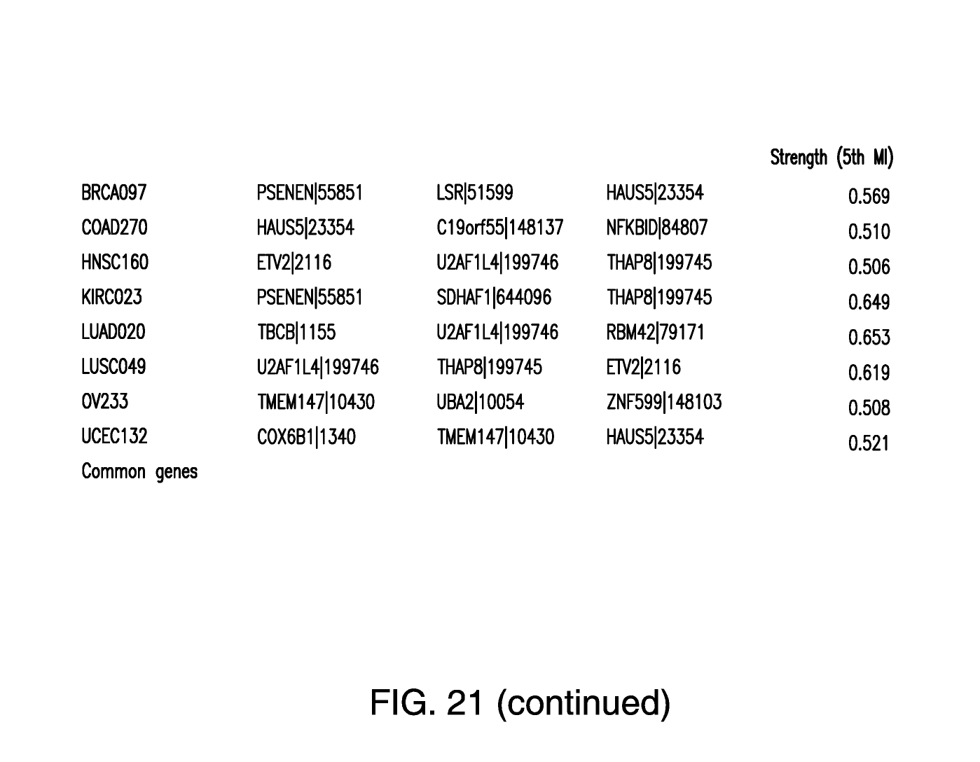

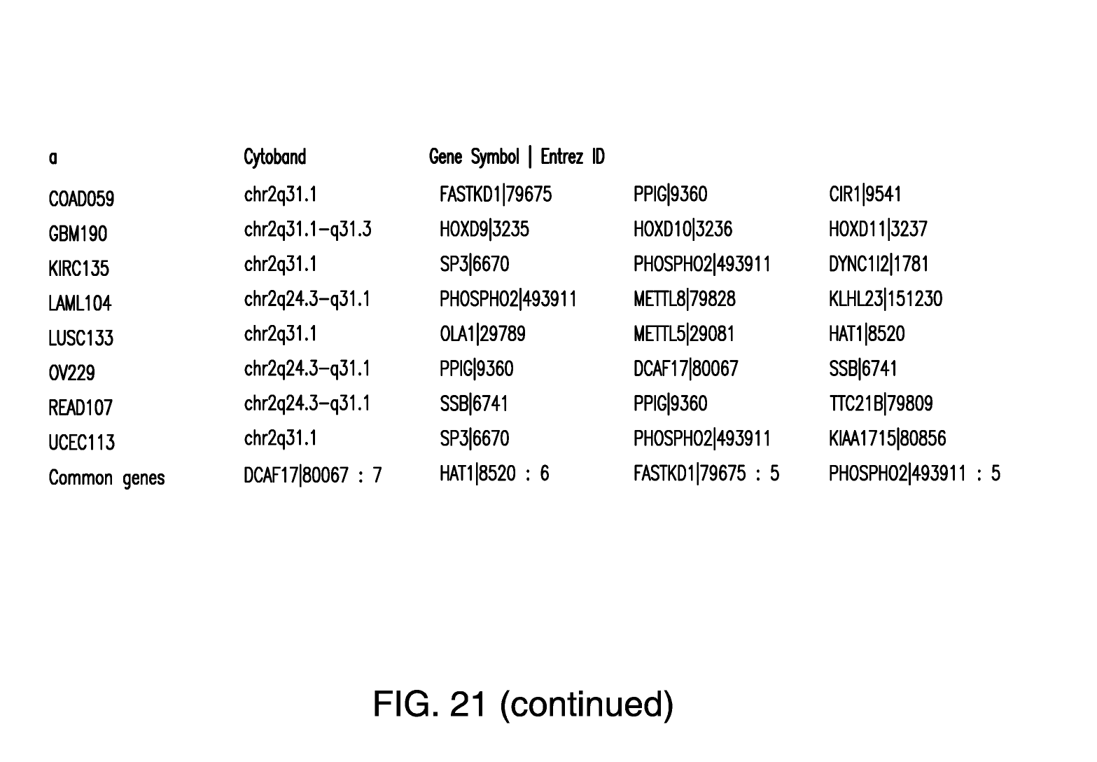

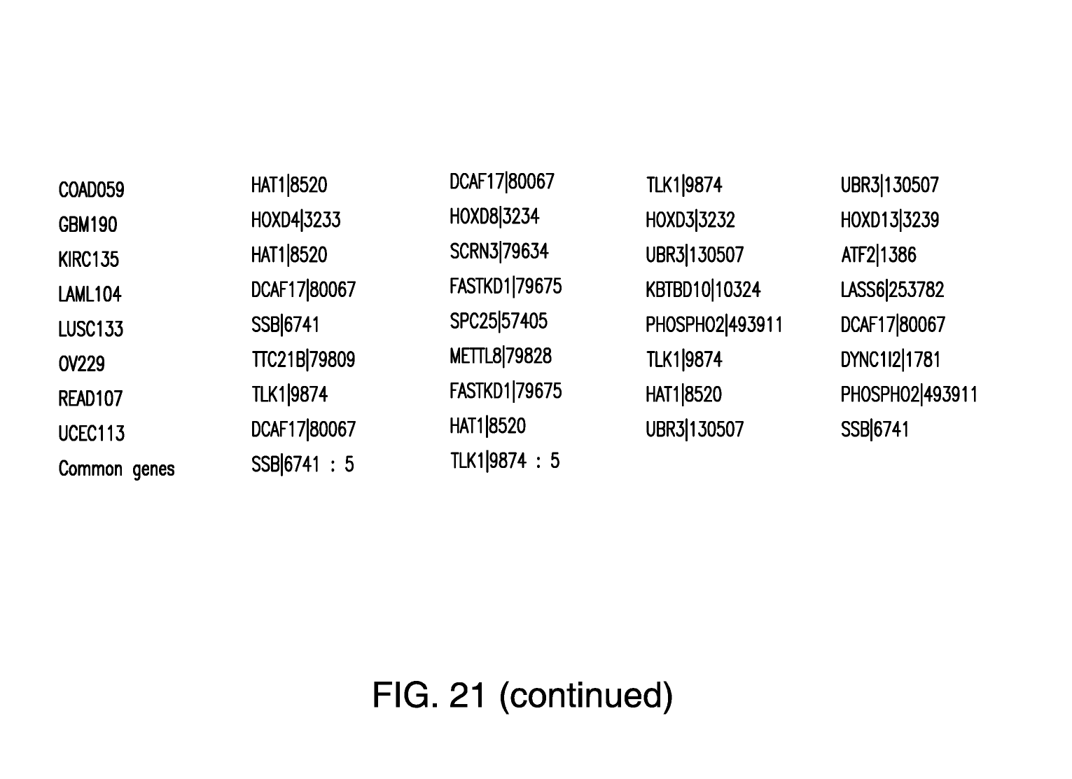

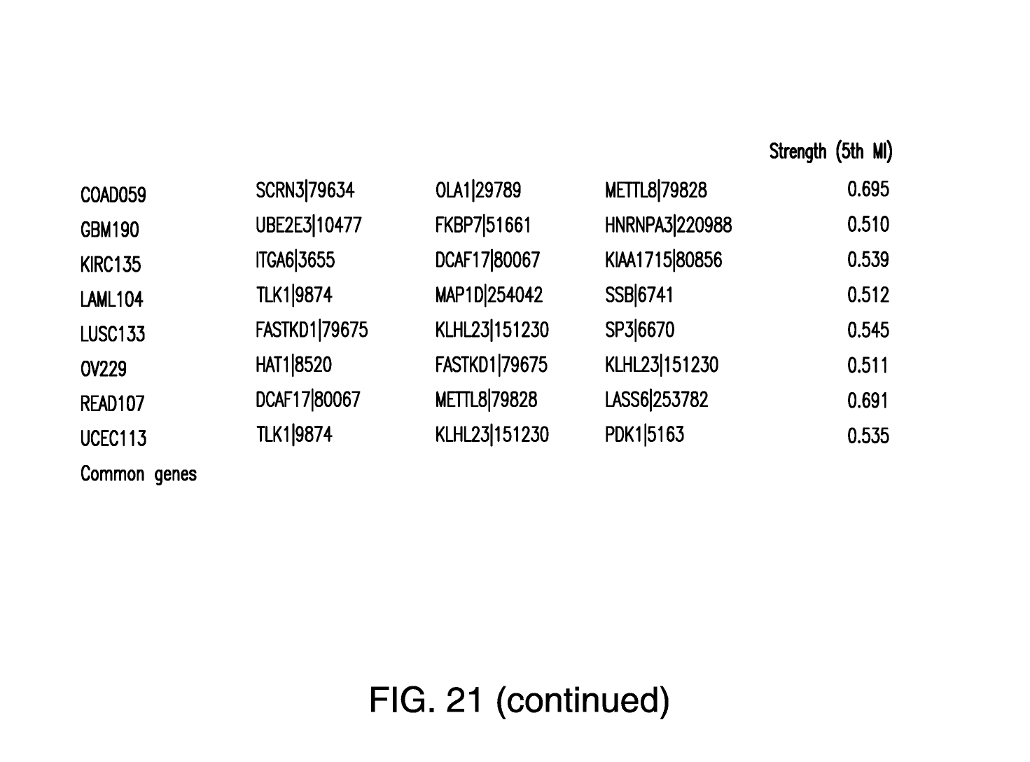

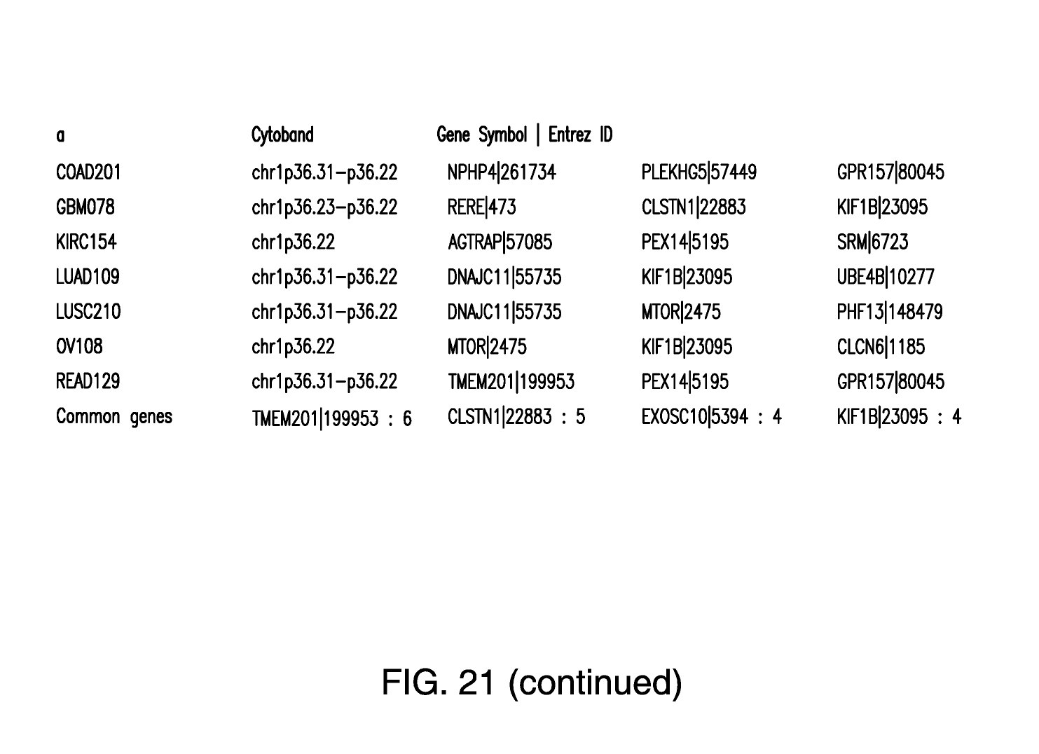

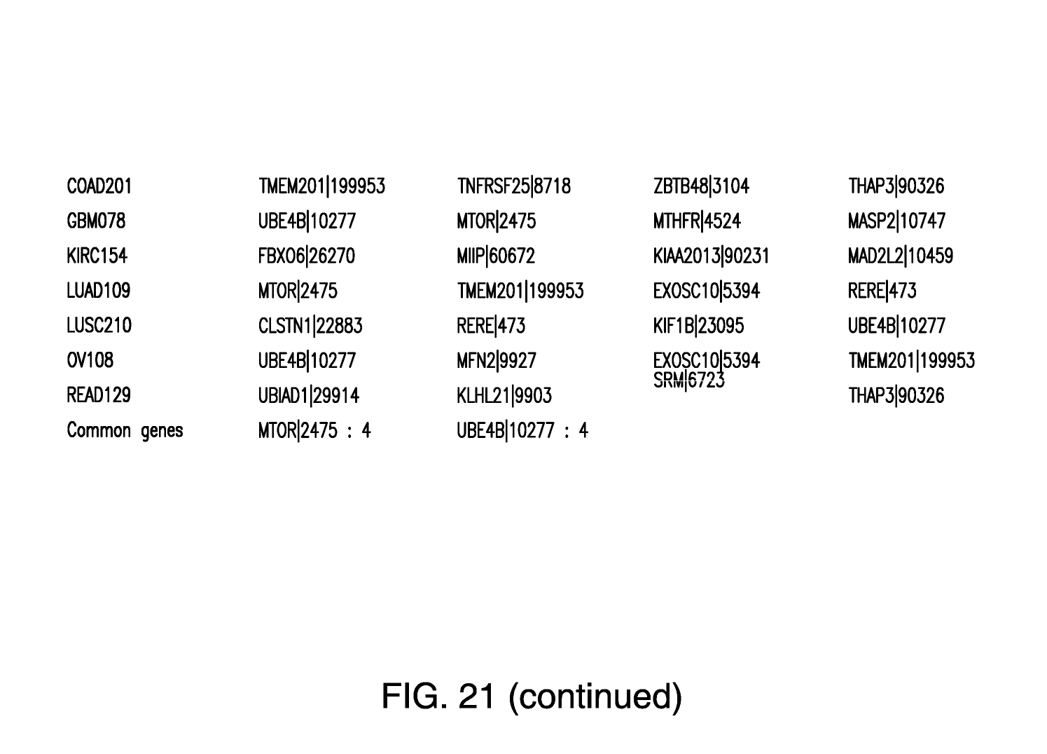

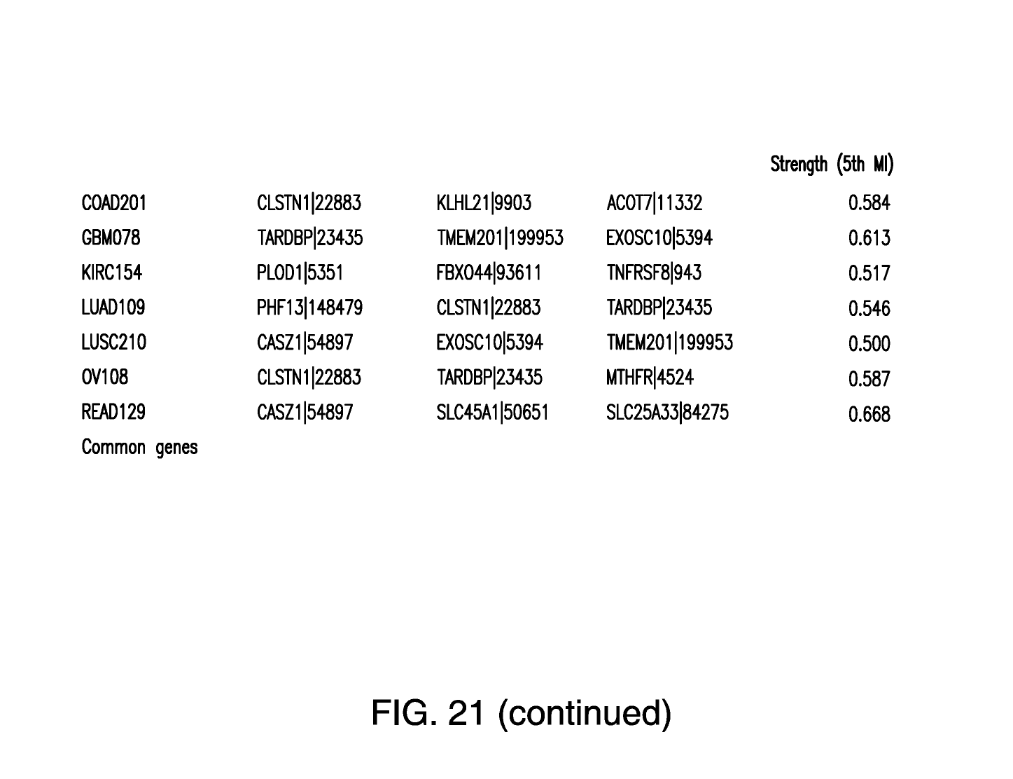

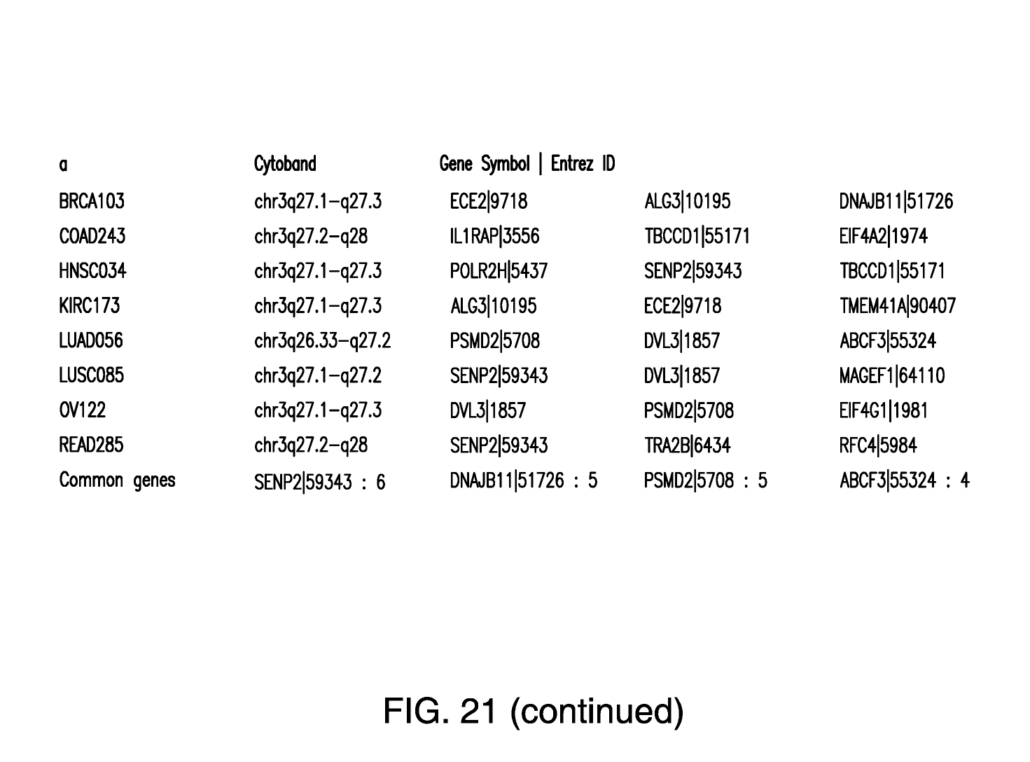

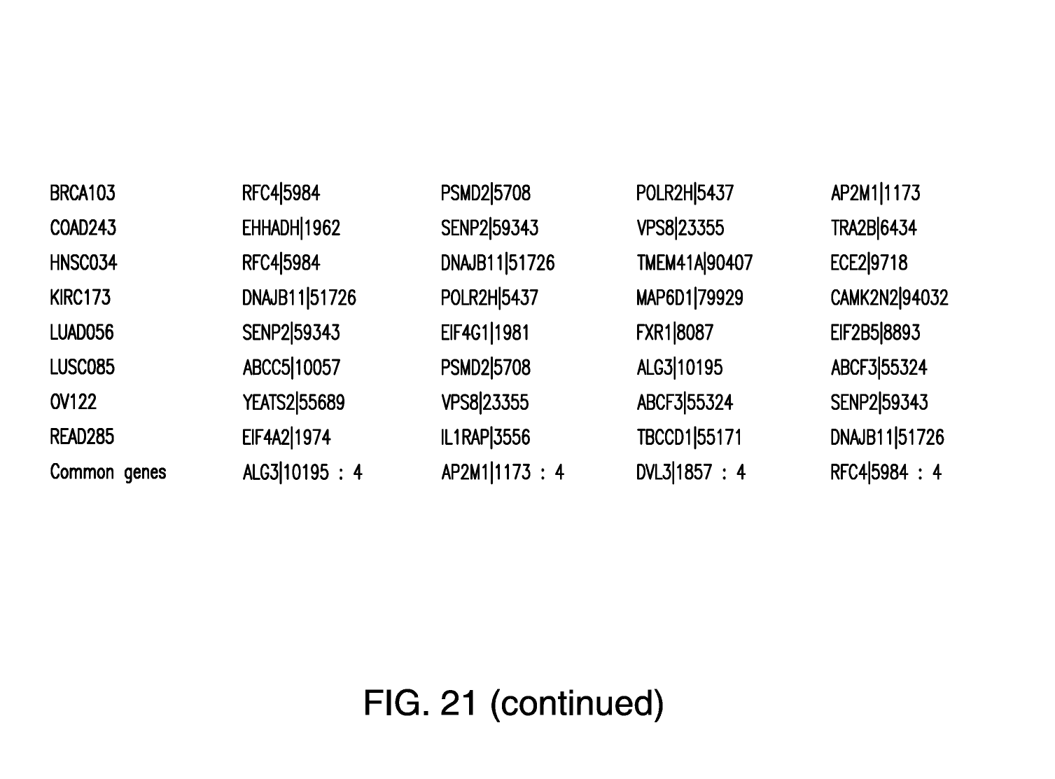

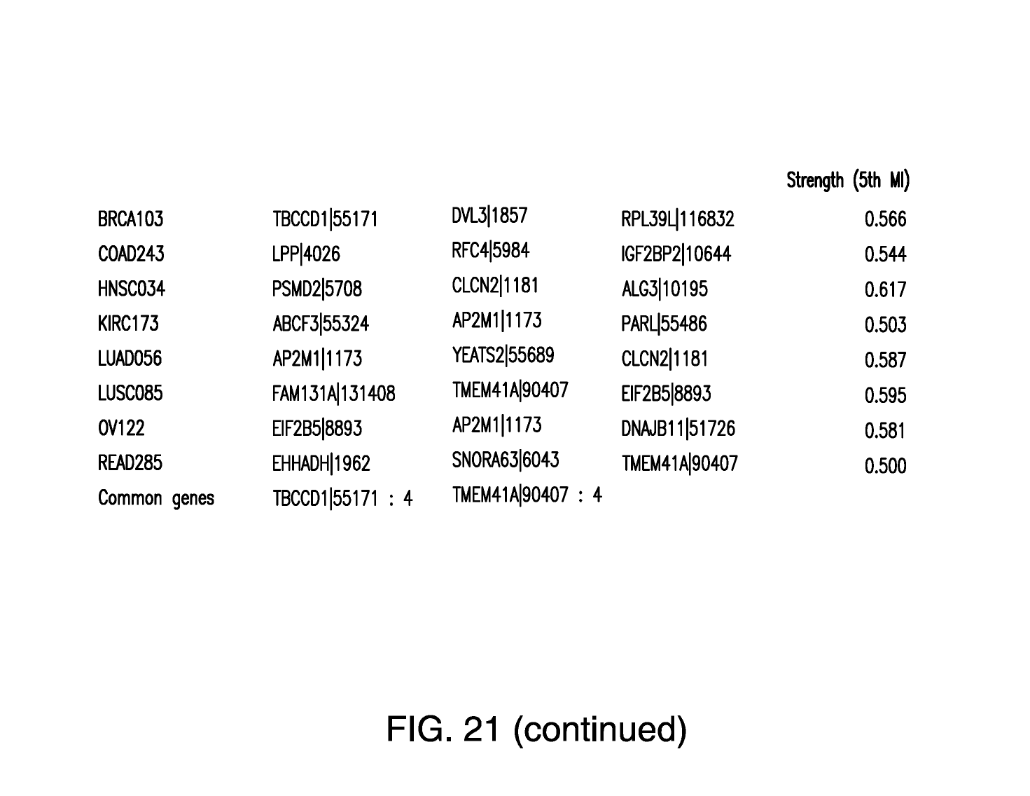

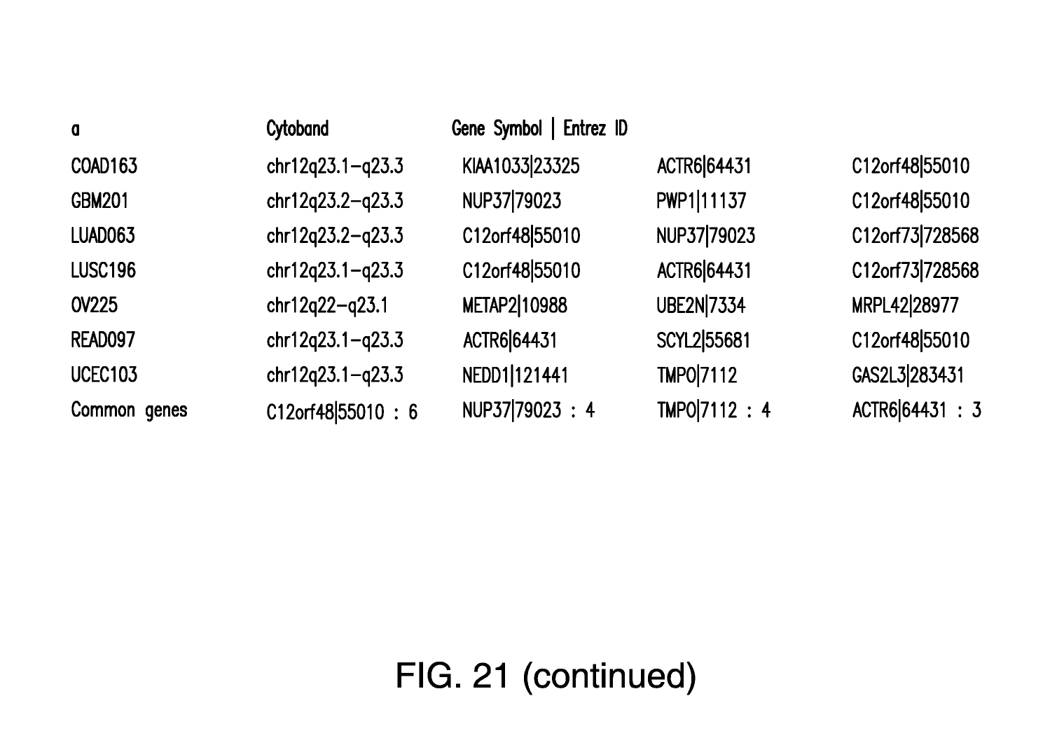

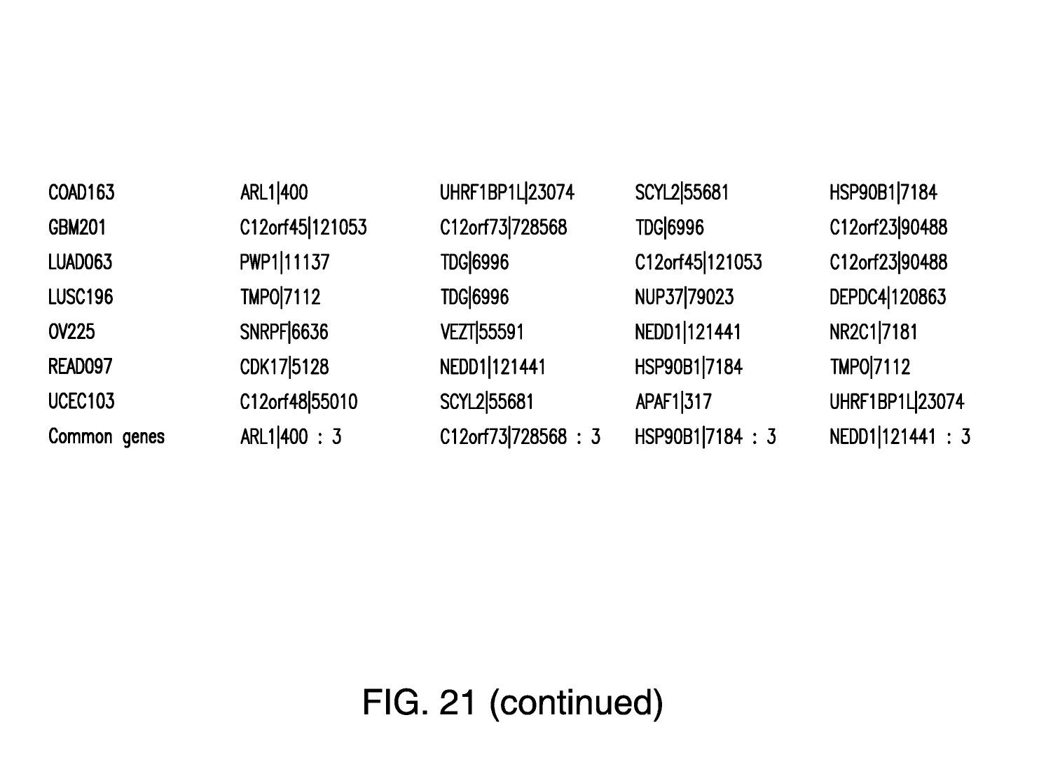

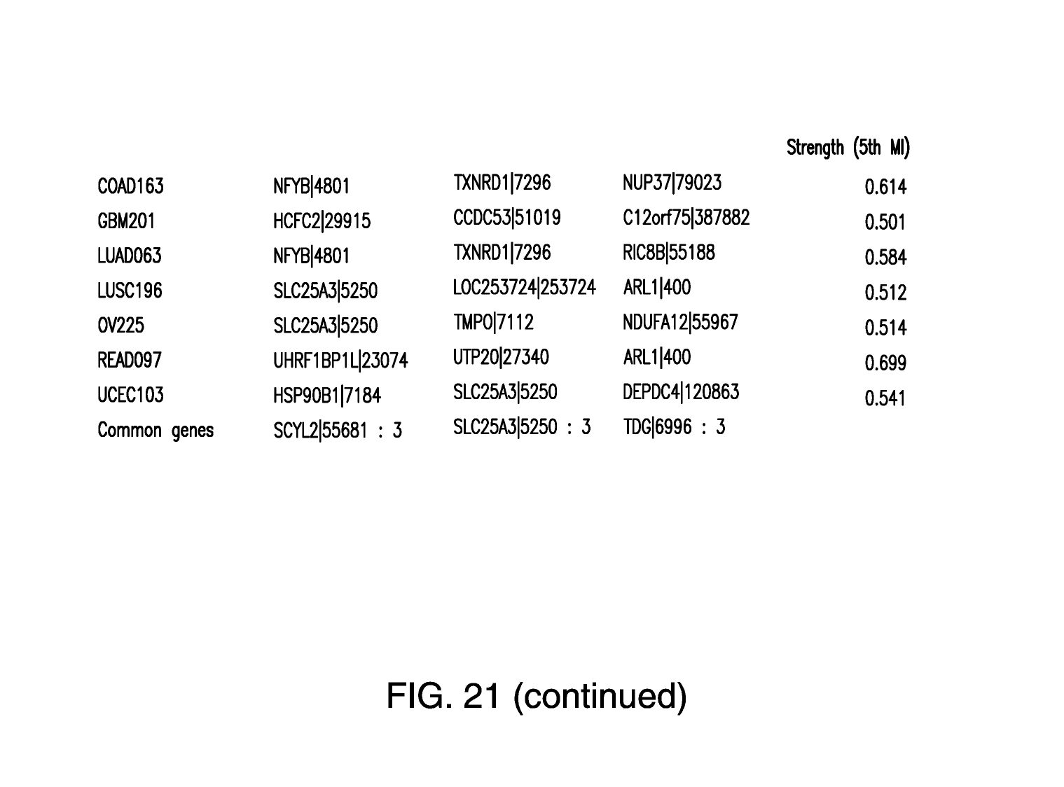

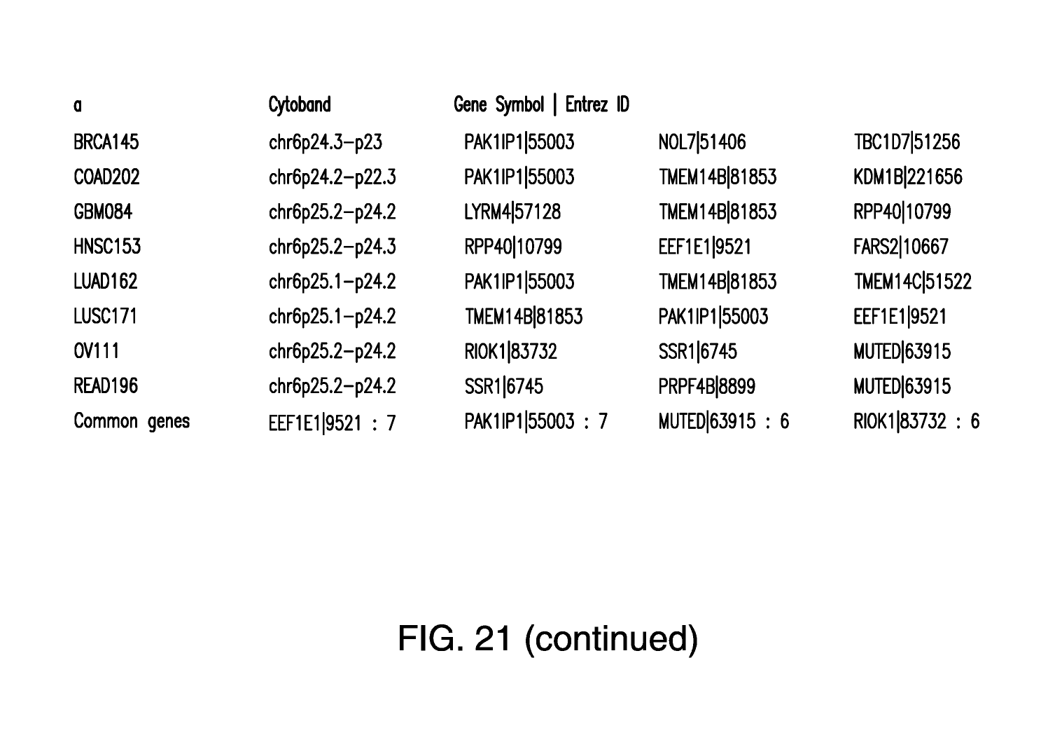

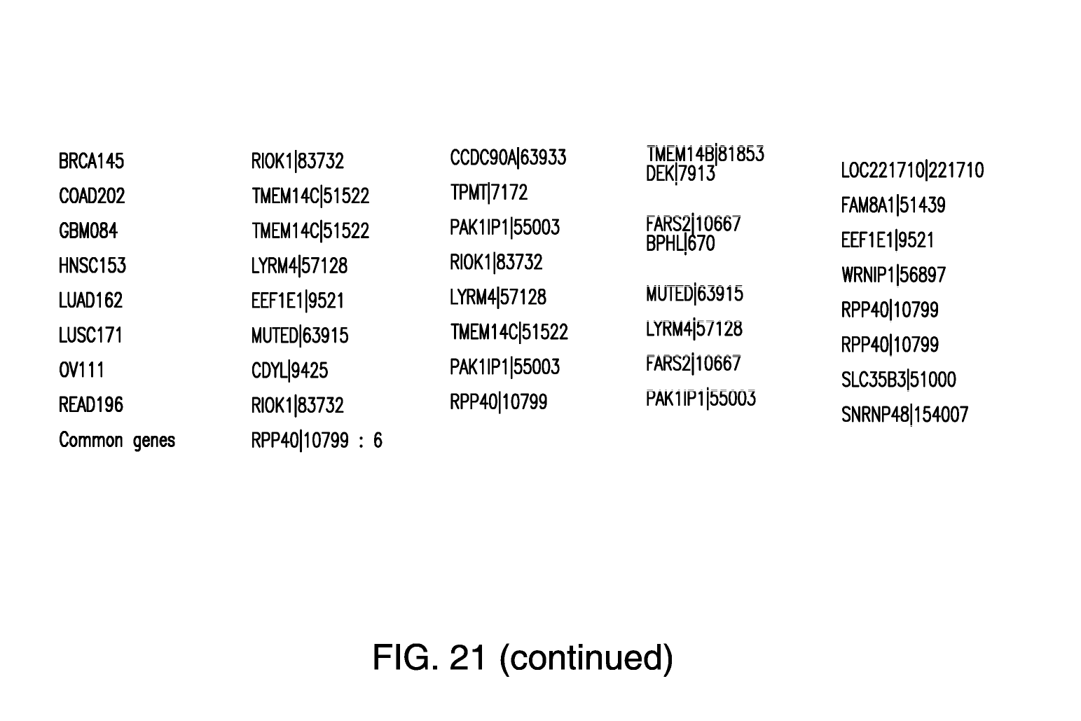

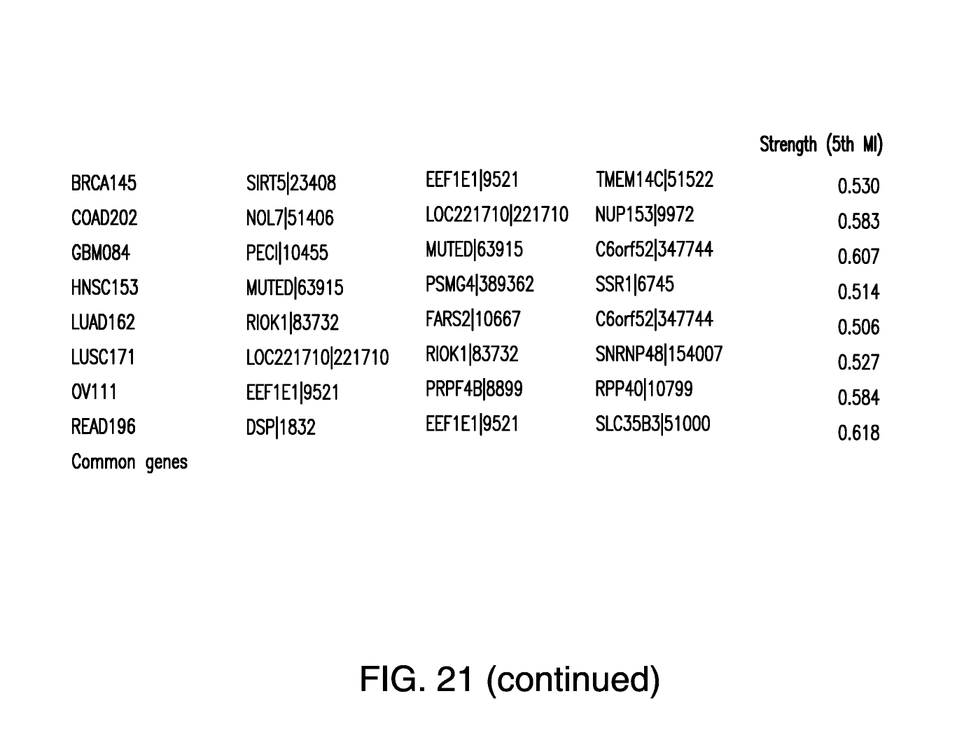

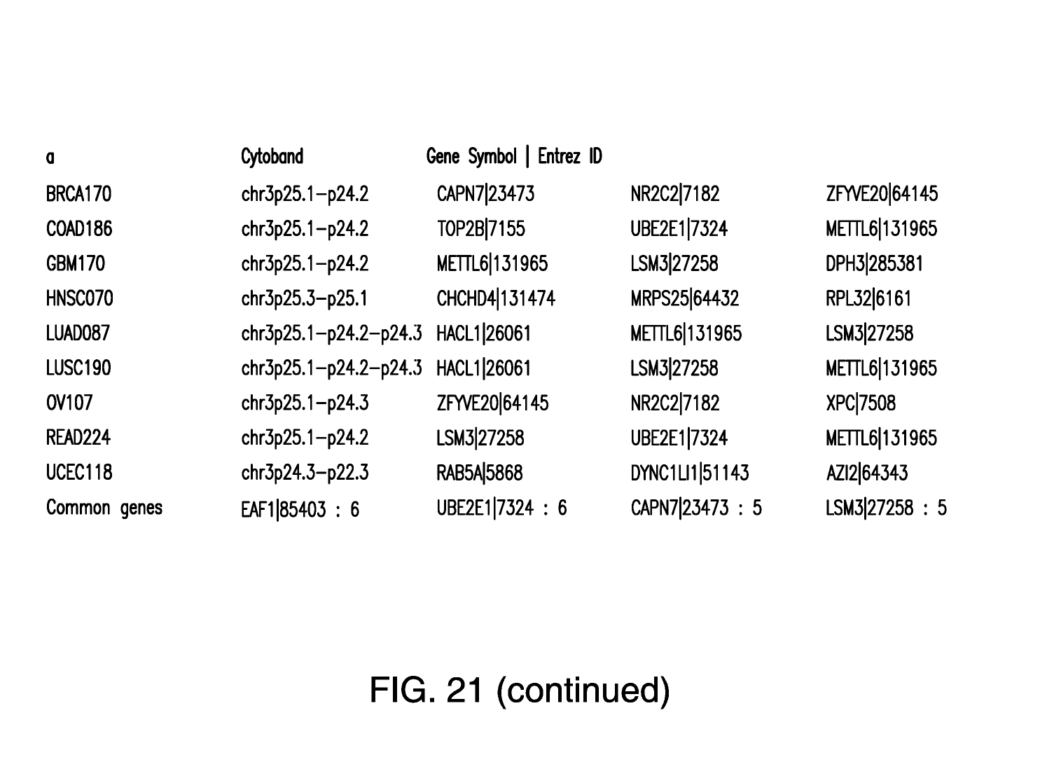

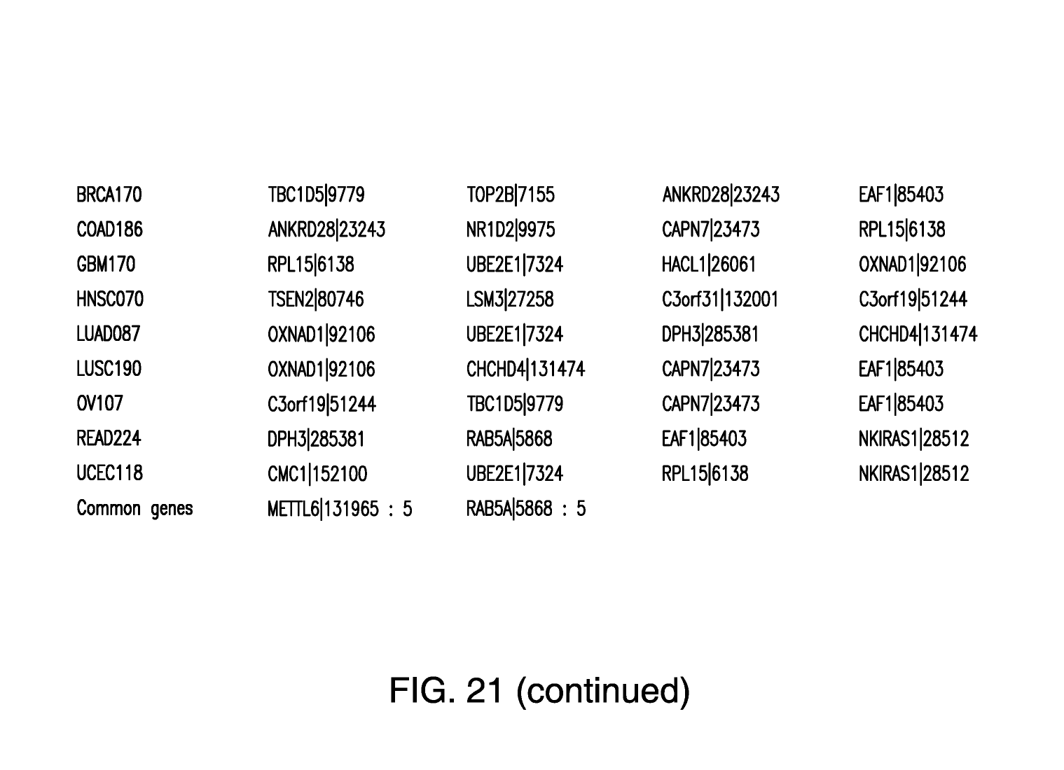

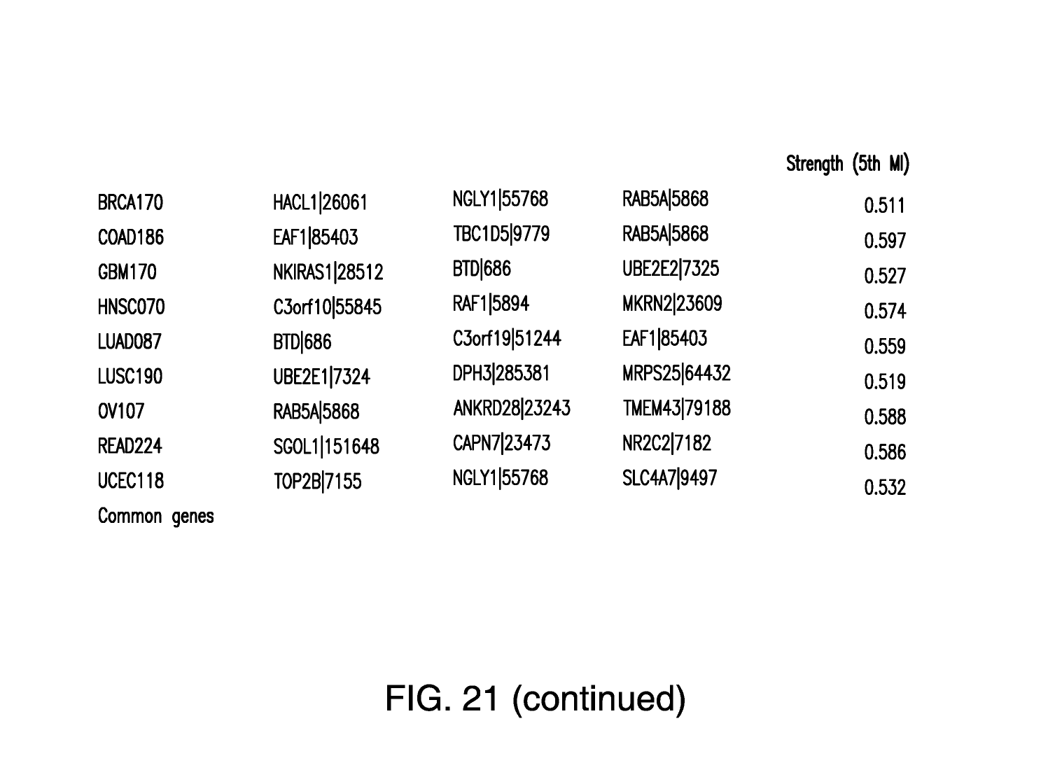

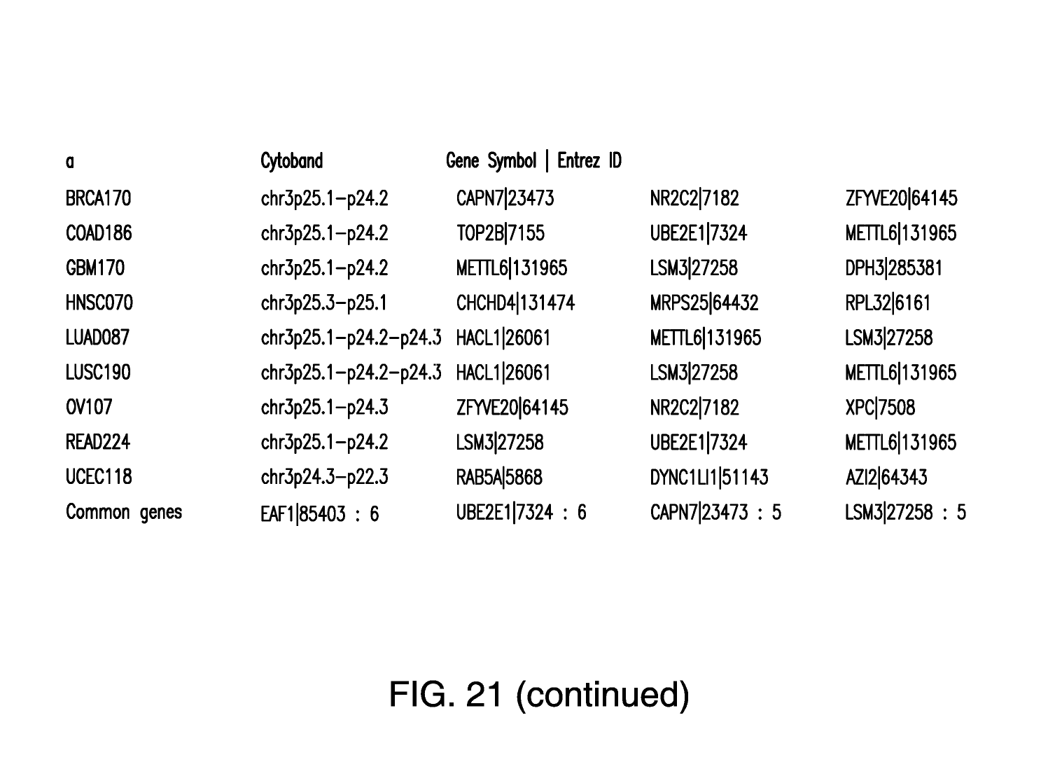

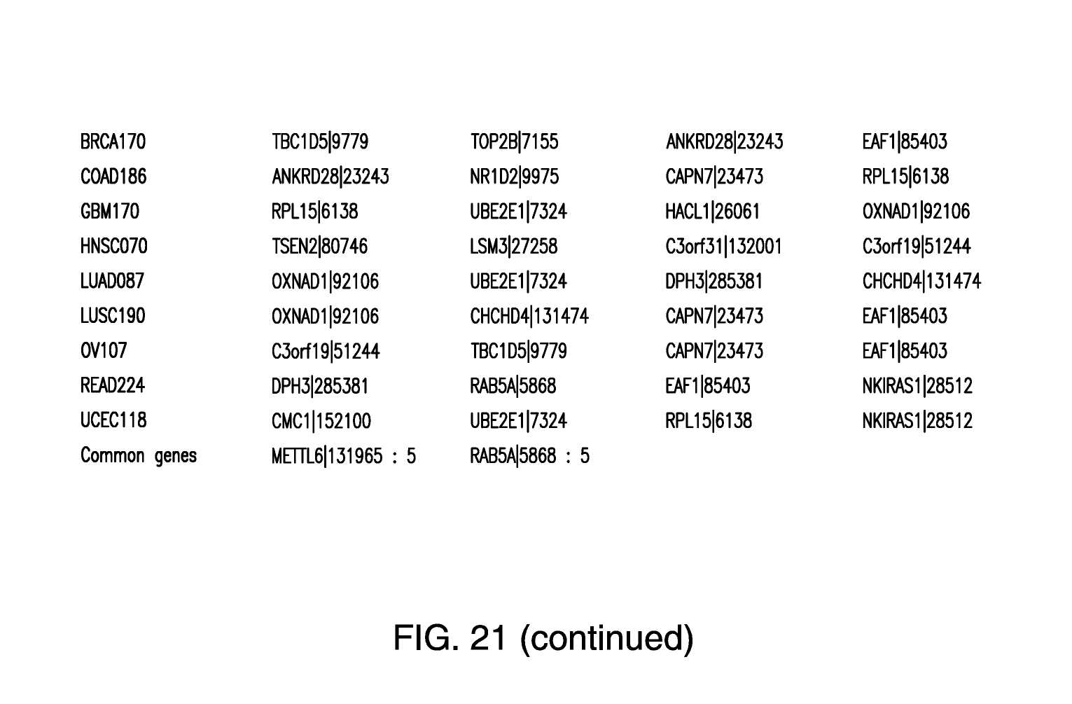

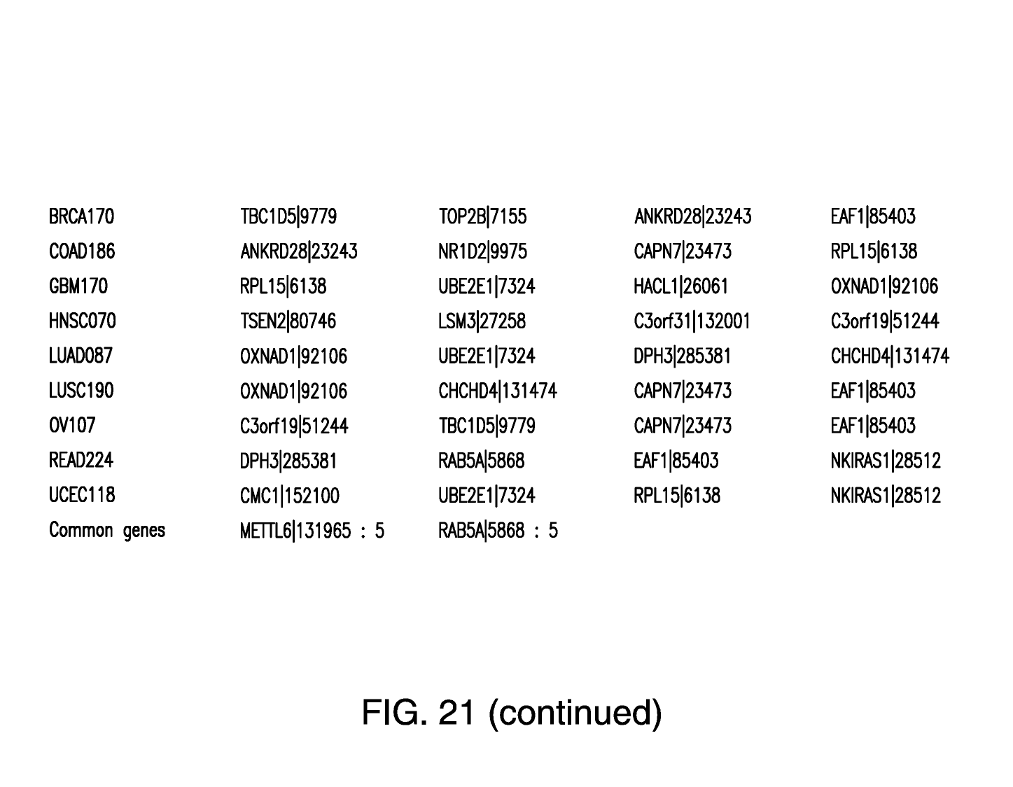

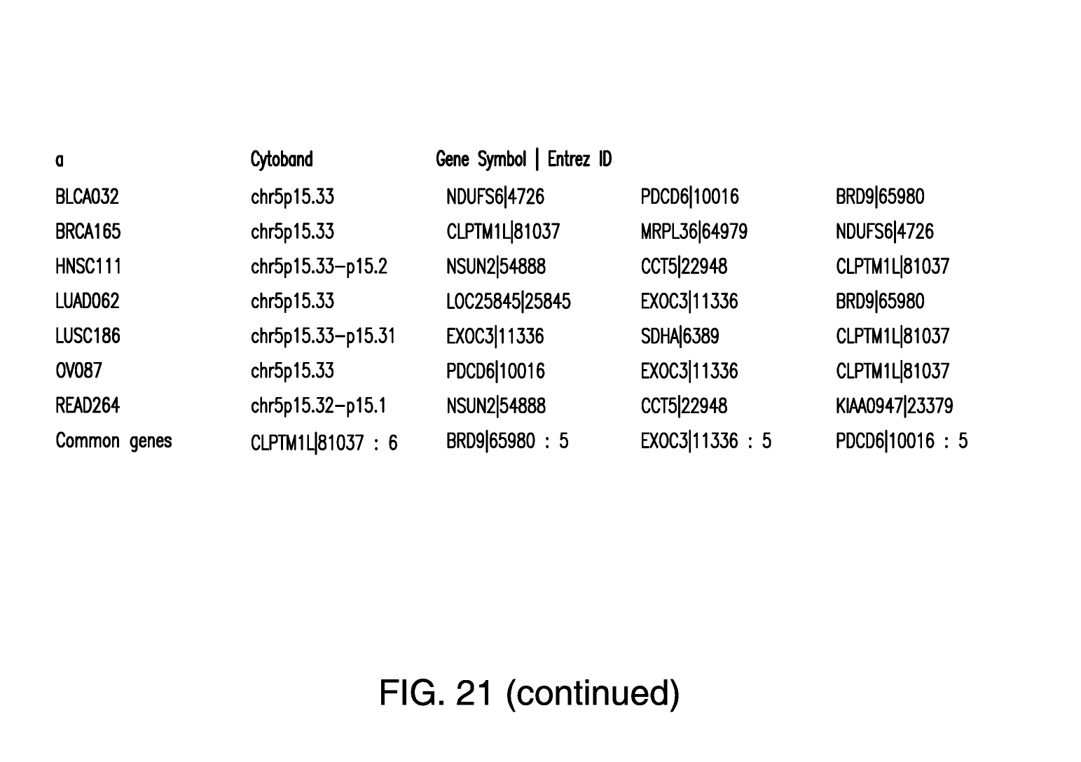

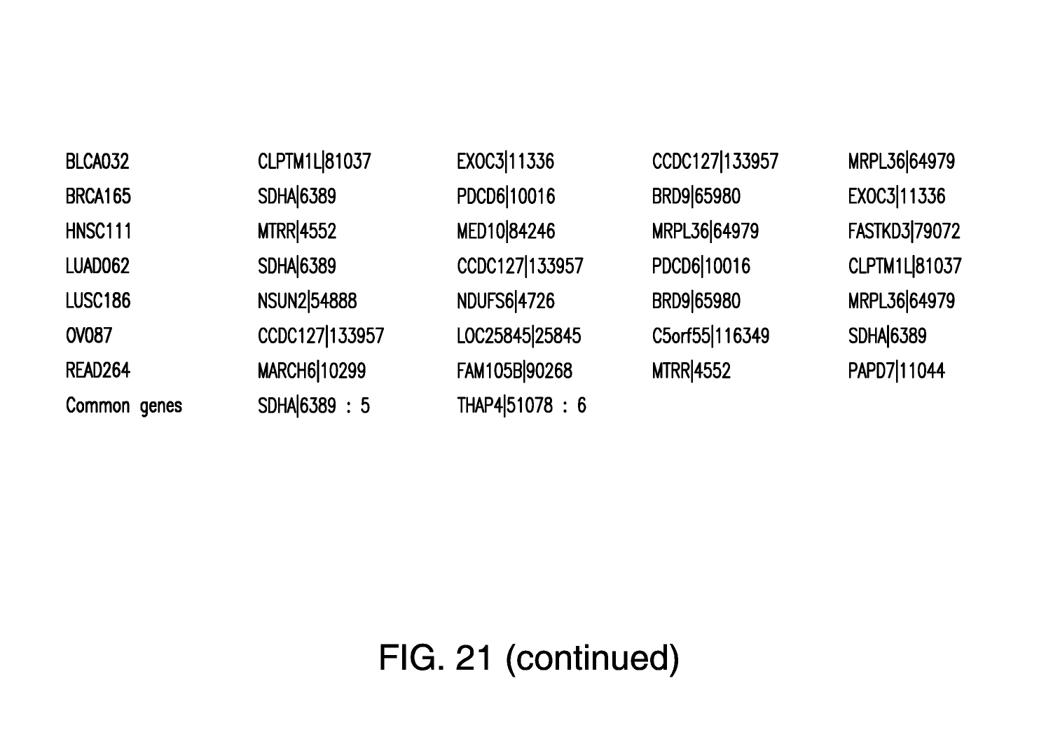

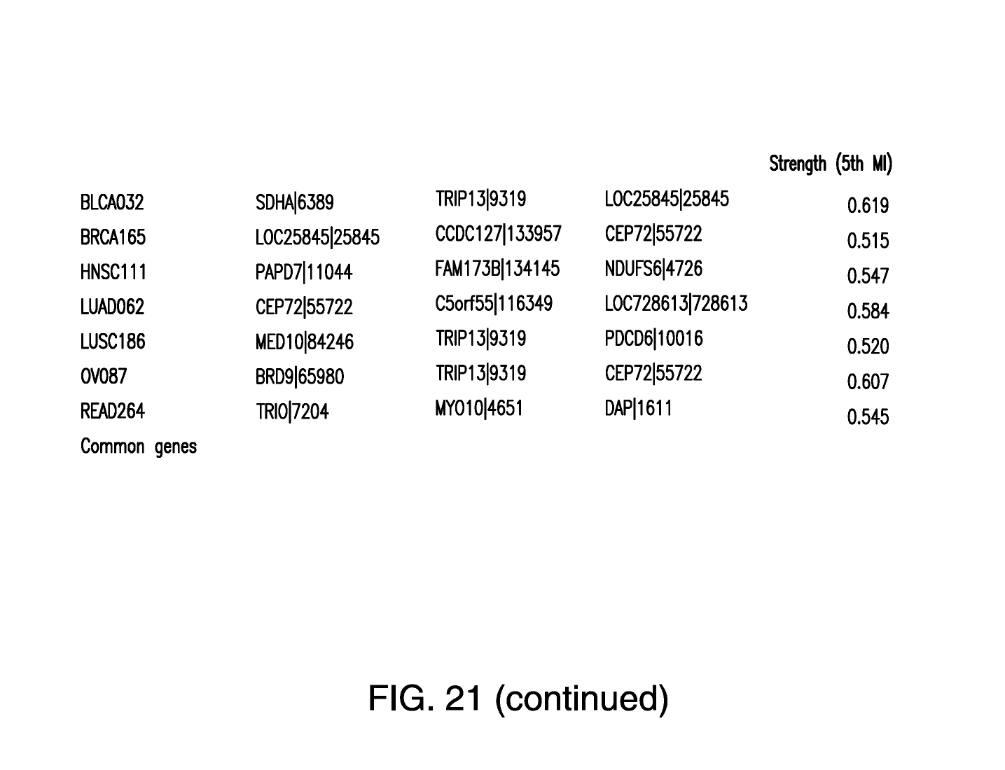

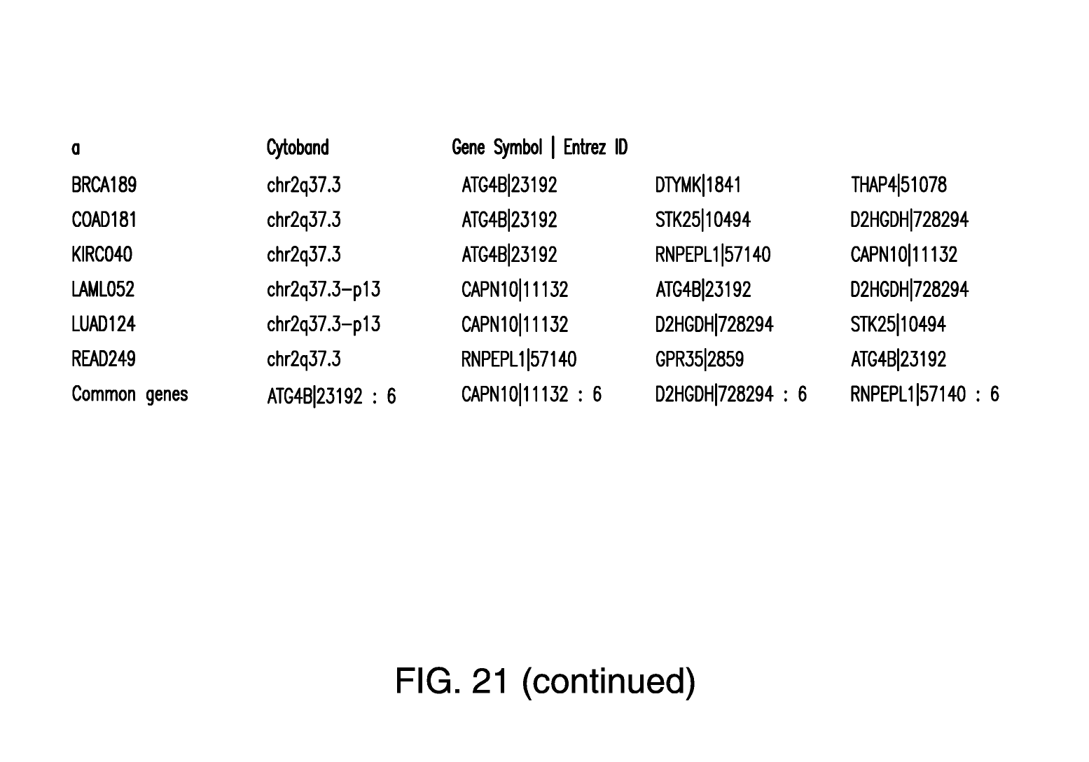

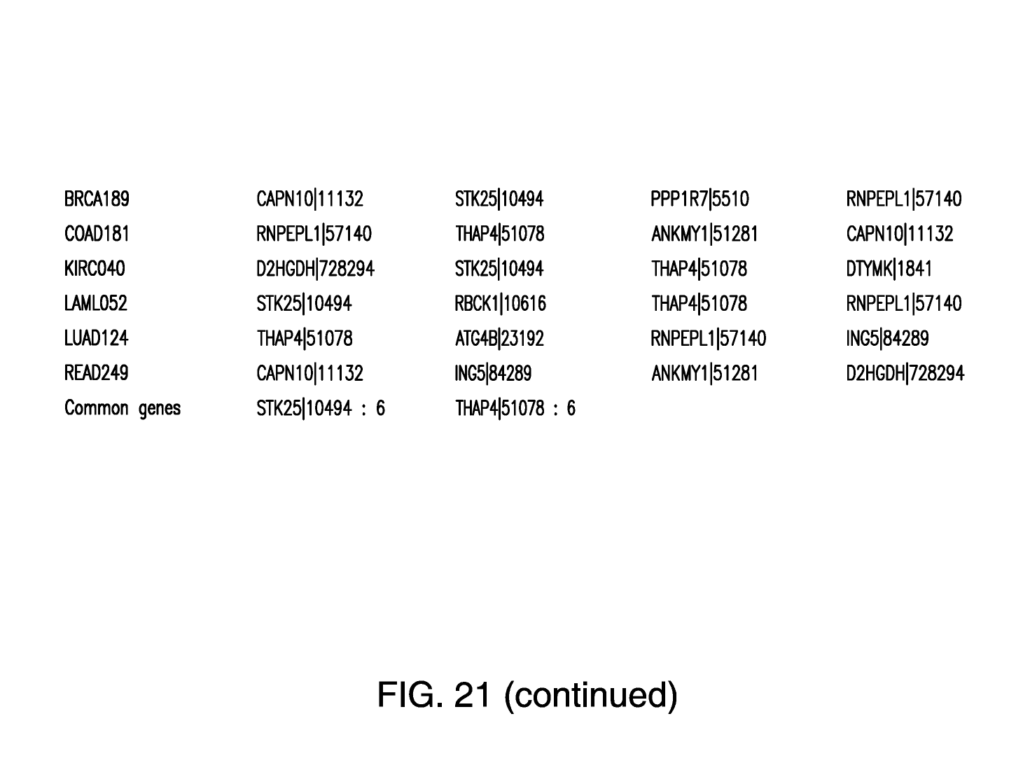

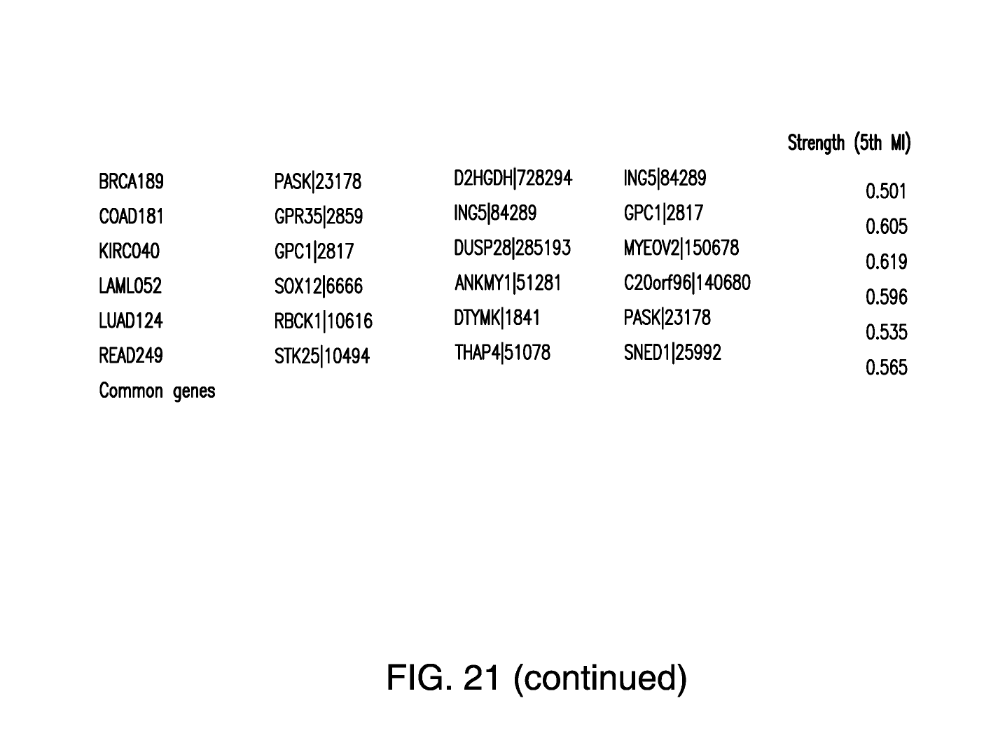

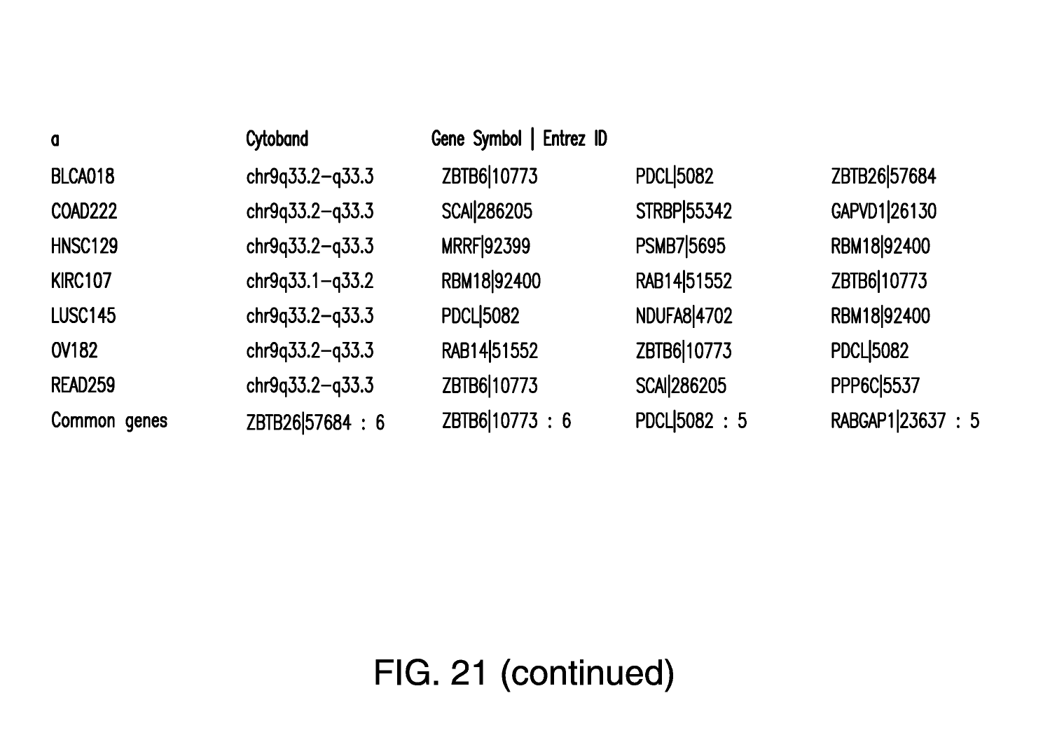

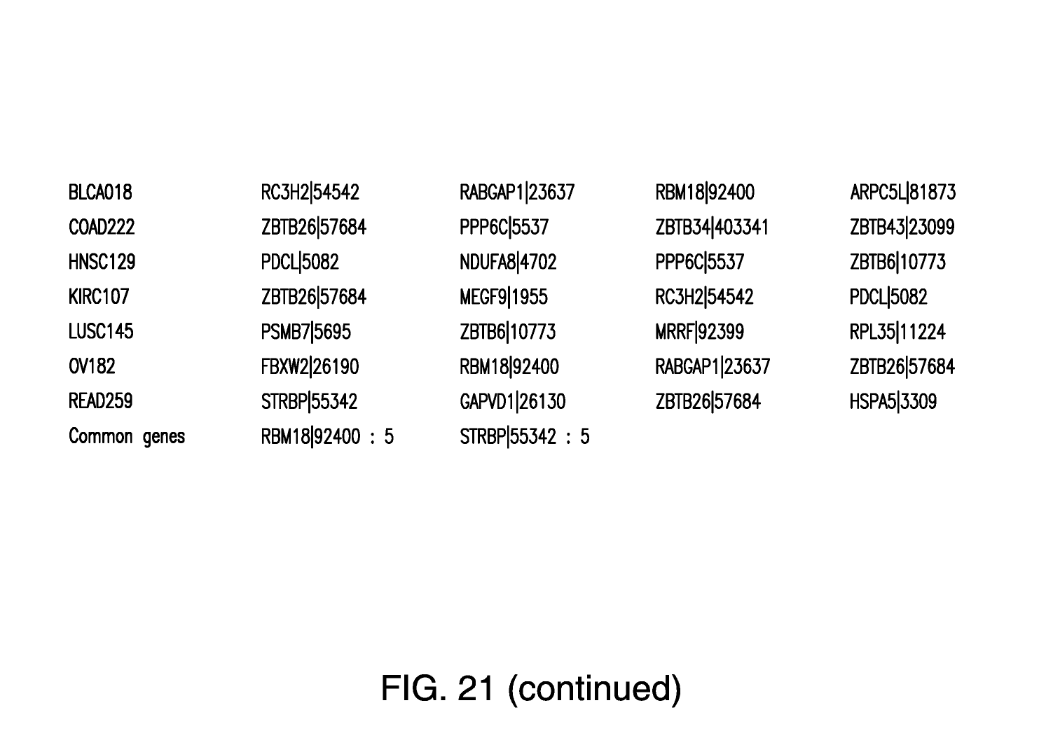

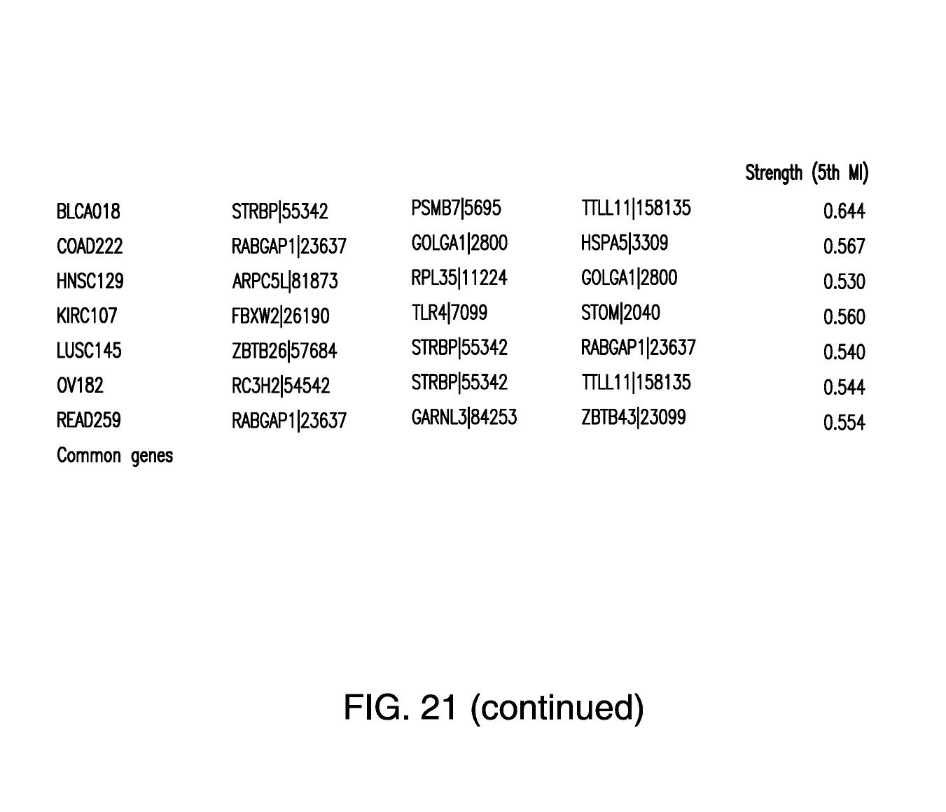

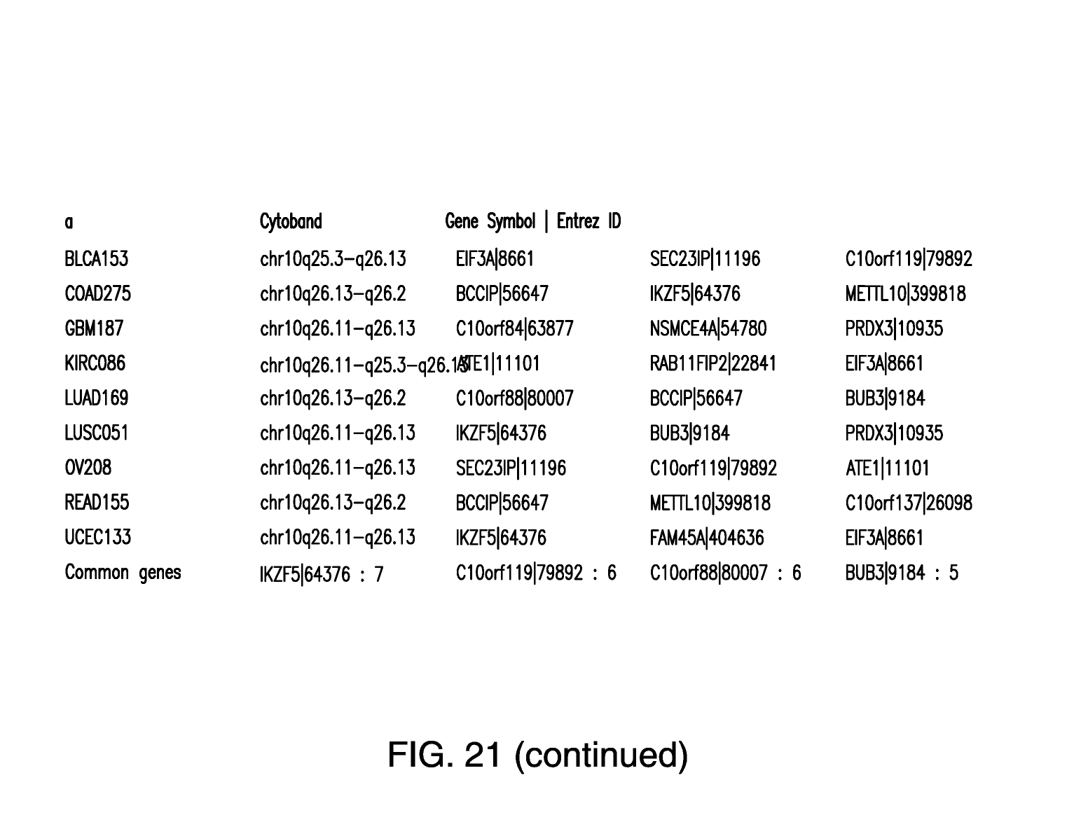

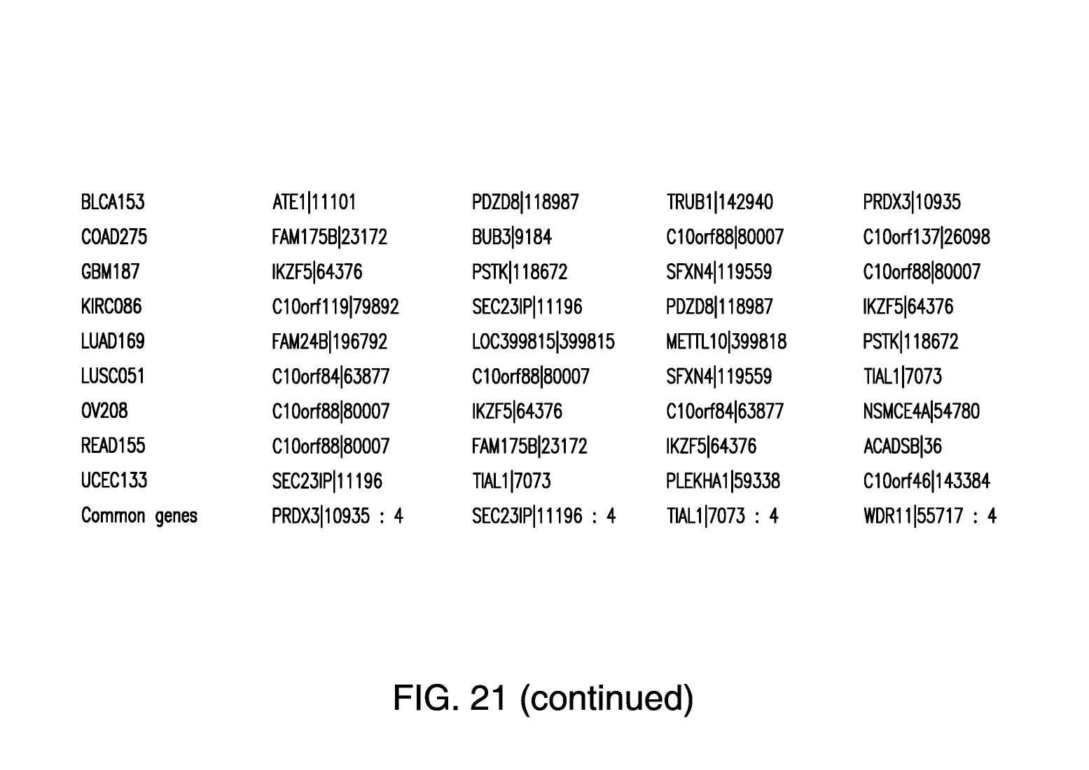

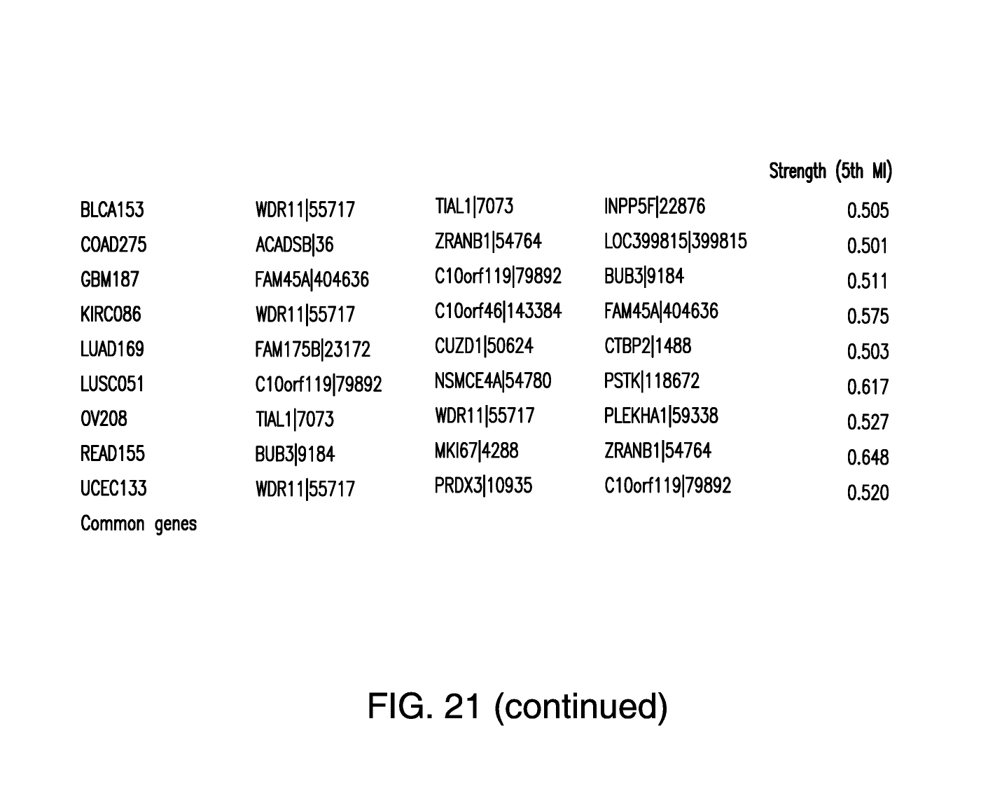

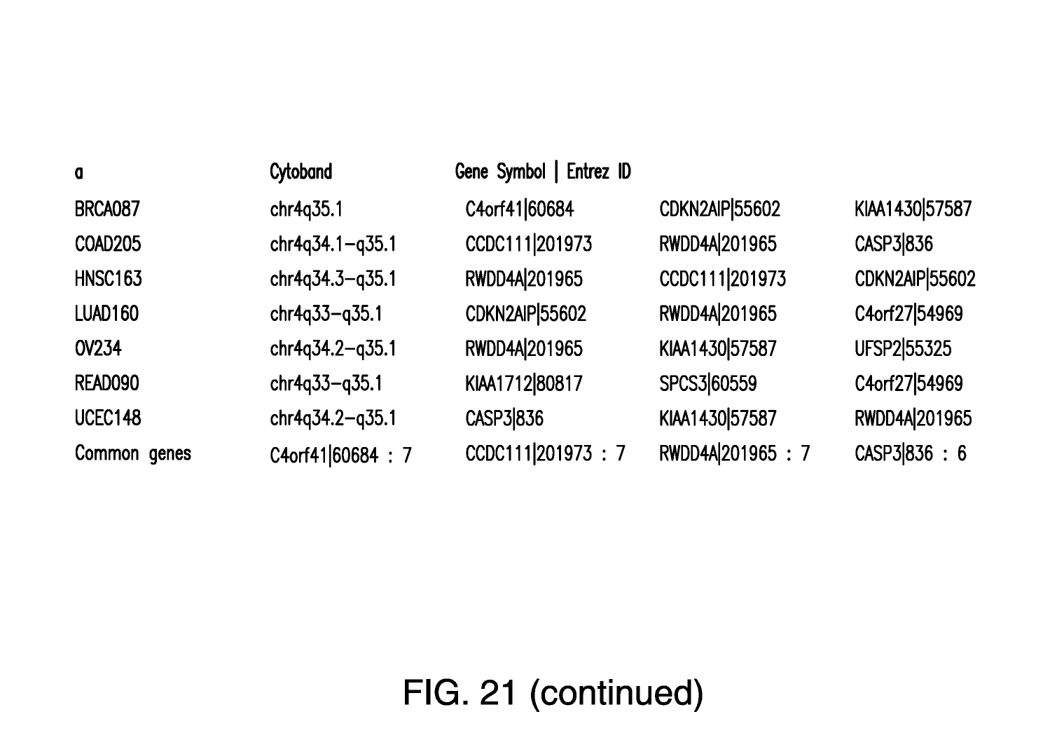

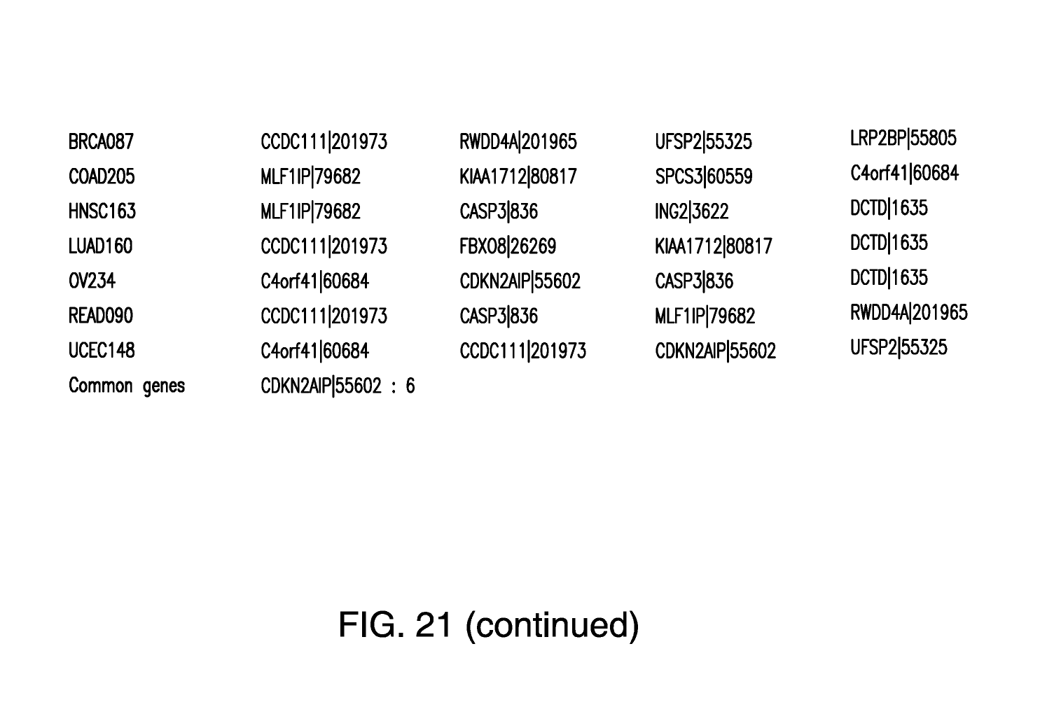

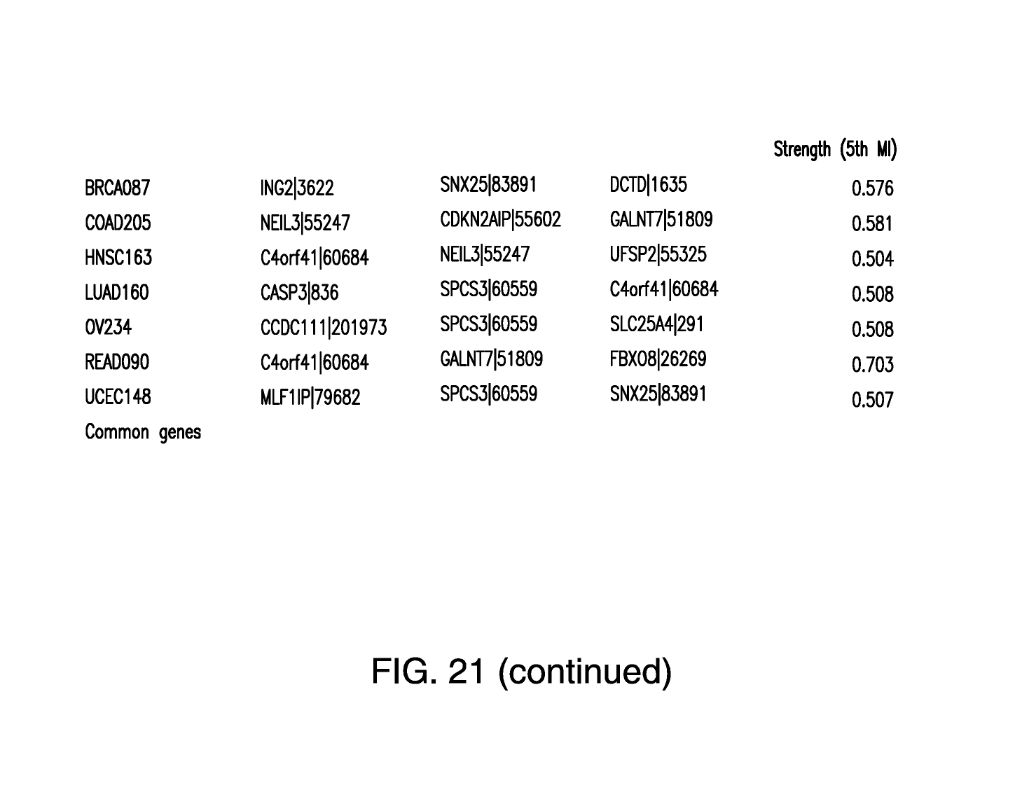

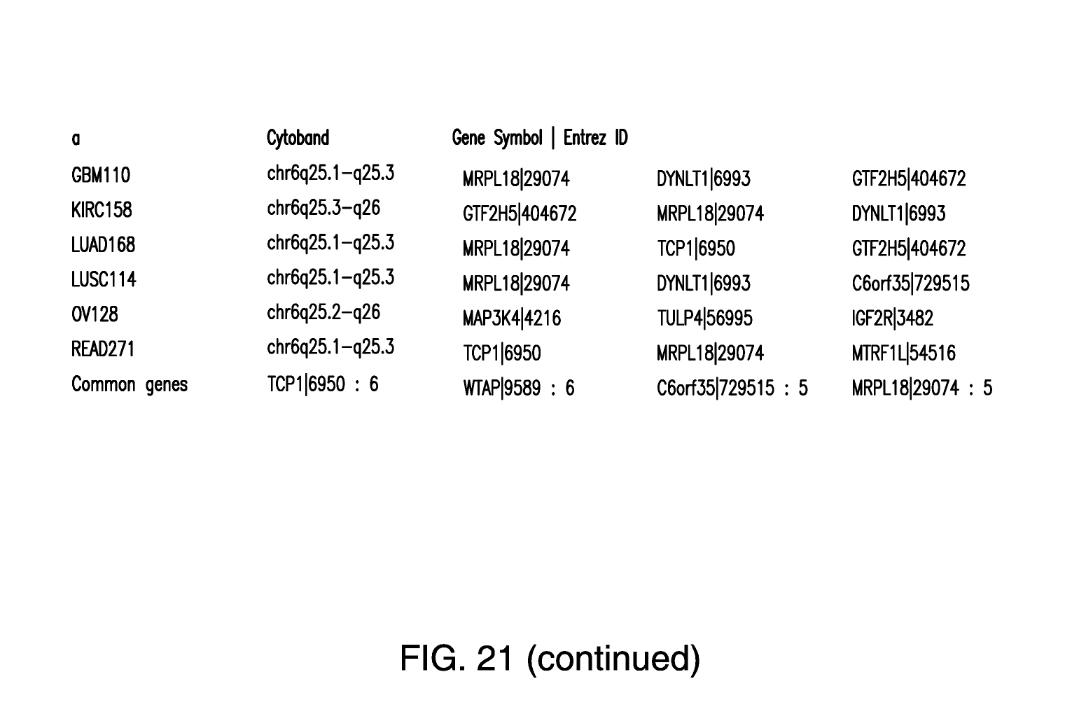

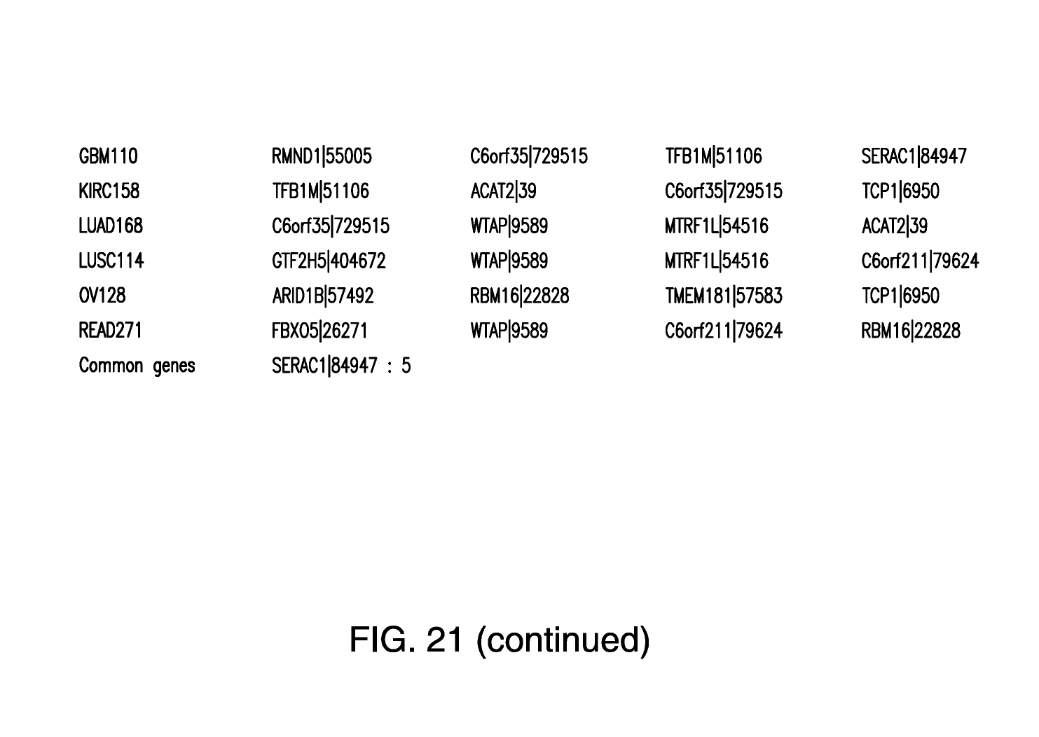

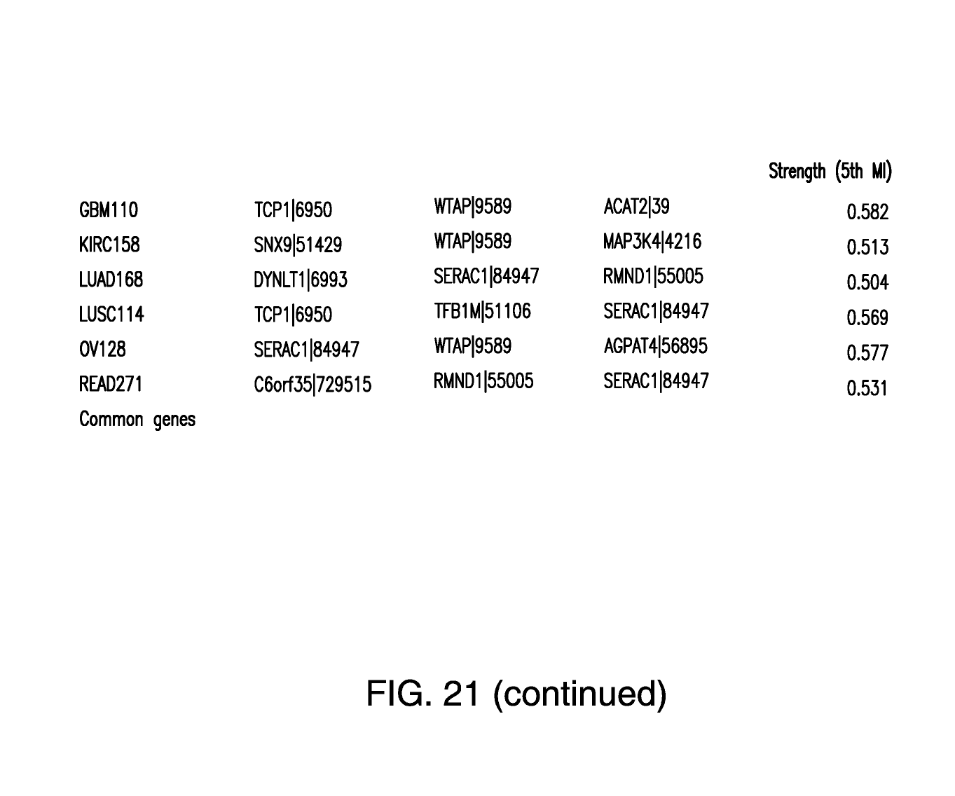

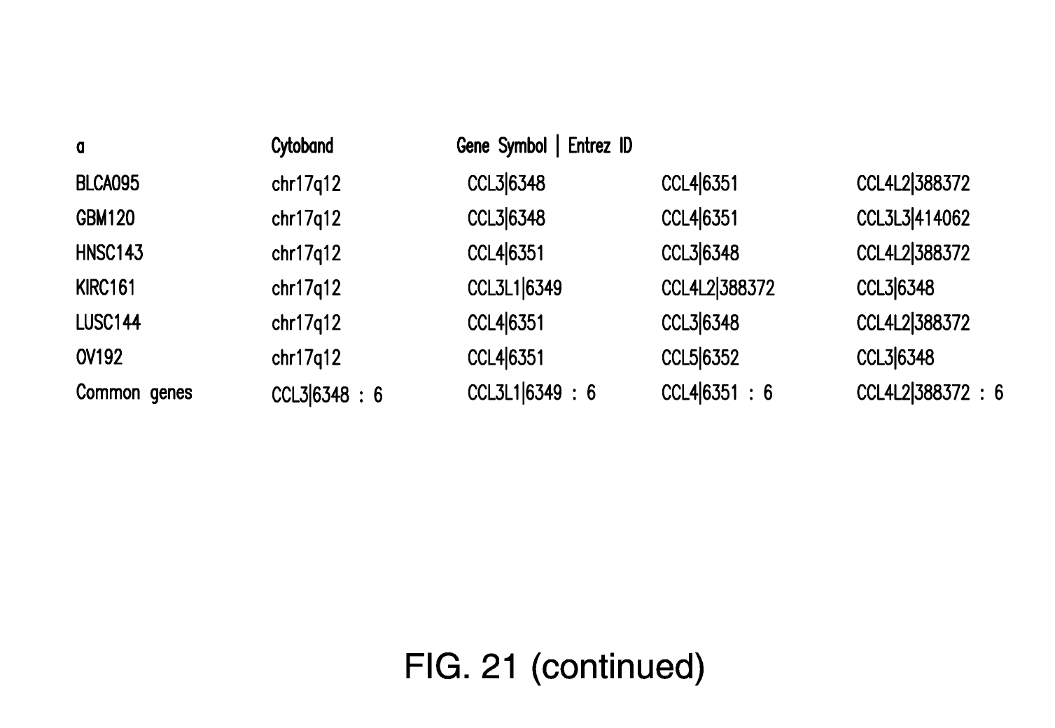

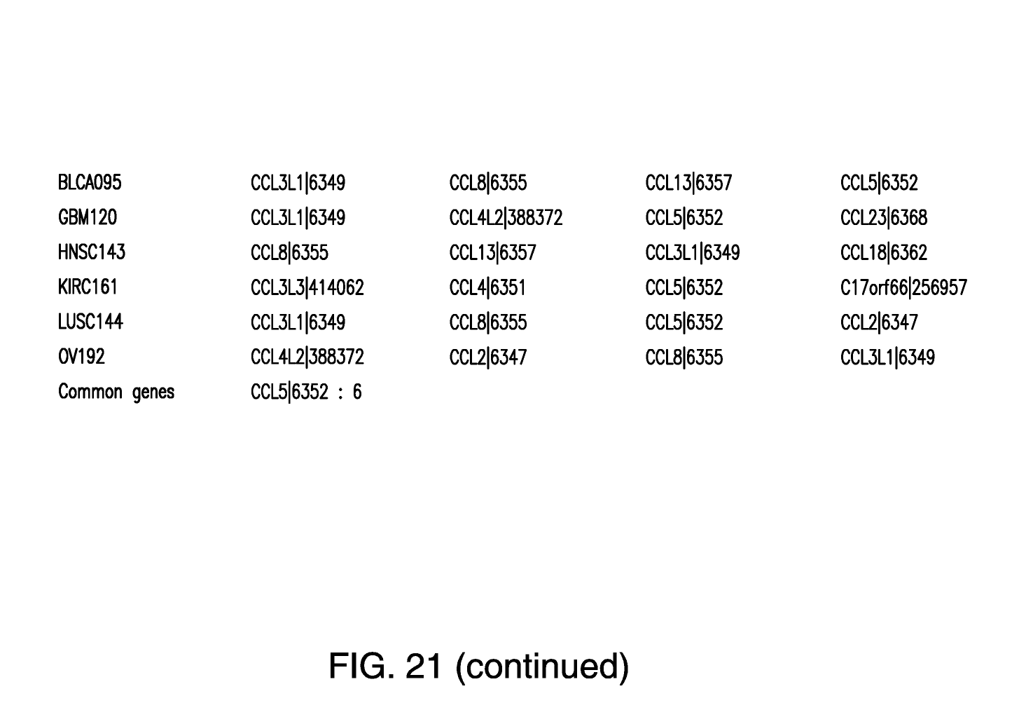

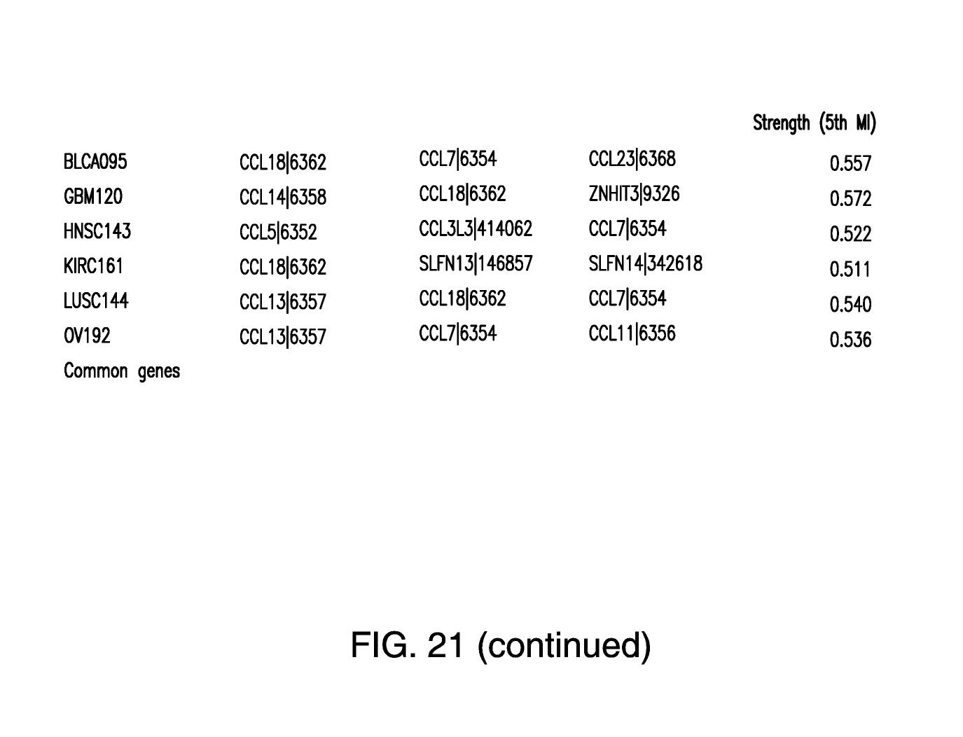

The instant example outlines the discovery of “pan-cancer” molecular signatures by applying computational methodology (see Materials & Methods, below) on the TCGA pancan12 data sets. Based on parameter choices that would guarantee that such signatures are clearly present in the majority of the data sets and would involve a significant number of mutually associated genes, 15 such attractor molecular signatures were identified, seven of which were present in protein-coding gene expression data sets, three in methylation data sets, three in microRNA expression data sets, and two in protein activity data sets. The attractor molecular signatures identified separately in individual cancer types are presented in FIGS. 19A-D. The consensus ranked lists for each of these signatures are presented in FIGS. 20A-D. Genomically localized molecular signatures were also identified, mainly representing amplicons, presented in FIGS. 21A-D.

### 5.1.2. Materials & Methods

### 5.1.2.1. Data Normalization

The data platform for each cancer types and its corresponding Synapse ID is given below.

| Molecular | | | | |
|---|---|---|---|---|
| profile | mRNA | Protein | miRNA | DNA methylation |
| Platform | Illumina | Reverse phase | Illumina HiSeq | Infinium |
| | HiSeq | protein lysate | | HumanMethylation27 |
| | | microarray (RPPA) | | BeadChip |
| Cancer type | Synapse ID | | | |
| BLCA | syn1571504 | syn1681048 | syn1571494 | syn1889358* |
| BRCA | syn417812 | syn1571267 | syn395575 | syn411485 |
| COAD | syn1446197 | syn416772 | syn464211 | syn411993 |
| GBM | syn1446214 | syn416777 | NA | syn412284 |
| HNSC | syn1571420 | syn1571409 | syn1571411 | syn1889356* |
| KIRC | syn417925 | syn416783 | syn395617 | syn412701 |
| LAML | syn1681084 | NA | syn1571533 | syn1571536 |
| LUAD | syn1571468 | syn1571446 | syn1571453 | syn1571458 |
| LUSC | syn418033 | syn1367036 | syn395691 | syn415758 |
| OV | syn1446264 | syn416789 | syn1356544 | syn415945 |
| READ | syn1446276 | syn416795 | syn464222 | syn416194 |
| UCEC | syn1446289 | syn416800 | syn395720 | syn416204 |

*The data sets were extracted from HumanMethylation450 BeadChip

For each RNA sequencing and miRNA sequencing data set, the mRNAs or miRNAs in which more than 50% of the samples have zero counts were removed from the data set. All the zero counts and missing values in the data sets were imputed using the k-nearest neighbors algorithm as implemented in the impute package in Bioconductor. The log2 transformed counts were then normalized using the quantile normalization methods implemented in Bioconductor's limina package. The missing values in the protein and DNA methylation data sets were also imputed using the k-nearest neighbors algorithm in the impute package. For the bladder and head and neck methylation data sets, for which only the Humanmethylation450 platform were provided, the 23,380 overlapping probes between the Humanmethylation27 and HumanMethylation450 platforms were extracted as new data sets for analysis.

### 5.1.2.2. Finding Attractors

The iterative algorithm for finding converged attractors was previously described (Cheng, W. Y., Ou Yang, T. H. & Anastassiou, D. PLoS Comput Biol 9, e1002920 (2013)) and is available as an R package under Synapse ID syn1123167. The parameters were used as described above. Specifically, the value of the exponent was selected to be a=5 for mRNA sequencing, and the same value for miRNA sequencing and for DNA methylation was used. For genomically localized attractors and for protein data sets due to their smaller dimension, the exponent a was set to 2. The strength of an attractor (to be used for attractor ranking as described below) was defined as the k^{th} highest mutual information among all genes with the converged attractor. For mRNA and methylation attractors, k was set at k=10, and for miRNA and protein attractors, k was defined as k=3, because it was observed that these attractors tend to consist of a smaller number of mutually associated elements.

### 5.1.2.3. Clustering Attractors of Different Cancer Types

After obtaining the converged attractors in each data set, a clustering algorithm was performed to identify extremely similar attractors across different cancer types, using the same algorithm as outlined above. The top features—mRNAs, miRNAs, proteins, or methylation probes—were used in each attractor as a feature set, then hierarchical clustering was performed on the feature sets across the cancer types, using the number of overlapping features as the similarity measure. The number of top features used to represent the attractor was chosen according to the distribution of the features' weights in the attractors. For the mRNA attractors, the top 20 features were used to create such feature sets. For the methylation attractors, top 50 features were used for clustering. For the miRNA and protein attractors, the top five features were used for clustering. A methylation attractor cluster containing sites exclusively on the X chromosome was removed, because its selection was gender-based. If an attractor cluster did not contain any gene that found in at least six cancer types, it was removed from consideration.

### 5.1.2.4. Creating Consensus Molecular Signatures

To account for the fact that some of the twelve data sets may not contain sufficient heterogeneous samples for showing each pan-cancer biomolecular event, the decision of selecting a signature was based on its clear presence in at least half of the cancer types, i.e., six different cancer types. A consensus molecular signature was thus created from each attractor cluster as follows: for each cluster, six significant attractors were identified by calculating the sum of the similarity measures (as defined above) between each attractor and all the other attractors, ranking the attractors using this quantity, and selecting the six top-ranked attractors. If an attractor cluster contained less than six attractors, it was removed from consideration. The average score for each feature across the six attractors was calculated and the features ranked accordingly as the consensus ranking. The ranking of the features is provided in FIGS. 20A-D.

### 5.1.2.5. Data Visualization

To create scatter plots for the top three features in the attractor, the values of the features on both axes were median-centered, so the median value for each feature in each data set is zero on the scatter plots. For the color-coded feature, the median was set to be gray, the minimum value to be blue, and the maximum value to be red, and interpolated the colors for intermediate values. For mRNA sequencing and miRNA sequencing data, the outlier values were removed, where the outliers were identified using the boxplot function in R.

### 5.1.2.6. Ranking Attractor Clusters

The strength of an attractor cluster was defined as the average strength of the six selected attractors in the cluster, as identified in the previous section. FIGS. 19-21 present the attractor clusters and their consensus rankings in the order of their corresponding attractor cluster's strength.

### 5.1.3. Results & Discussion

The three main attractor metagenes (CIN, MES, LYM) that had been previously identified were confirmed as the most prominent ones in the gene expression data sets. Additionally, several new molecular signatures resulting from this new thorough analysis were identified, one of which (END) contains endothelial markers and is associated with angiogenesis.

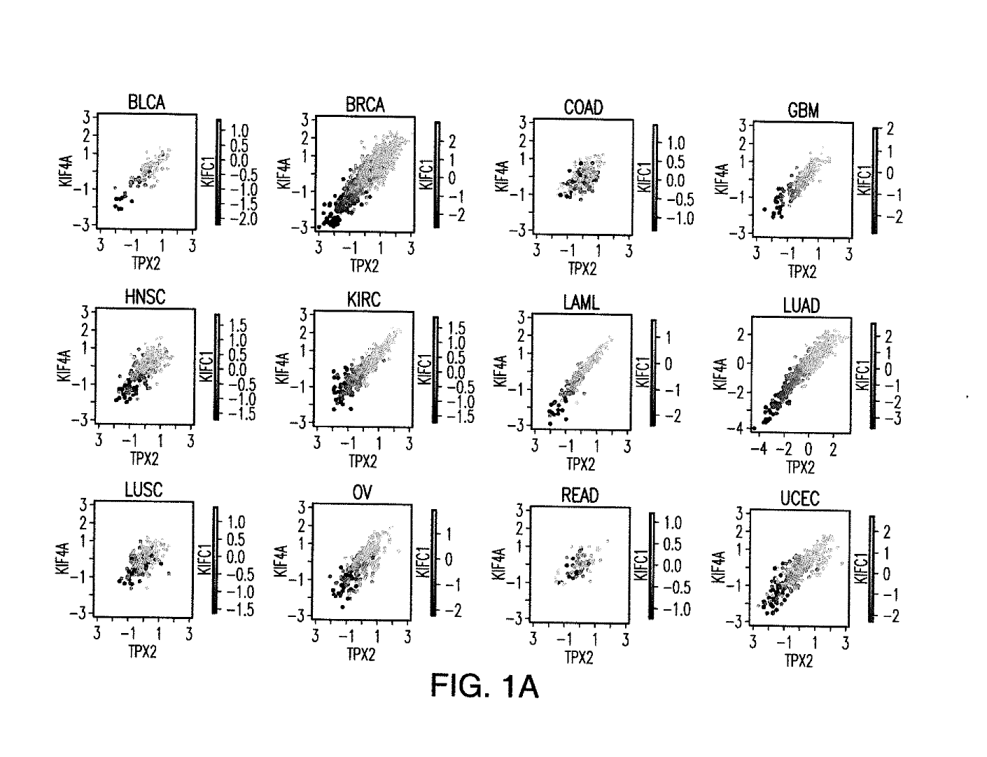

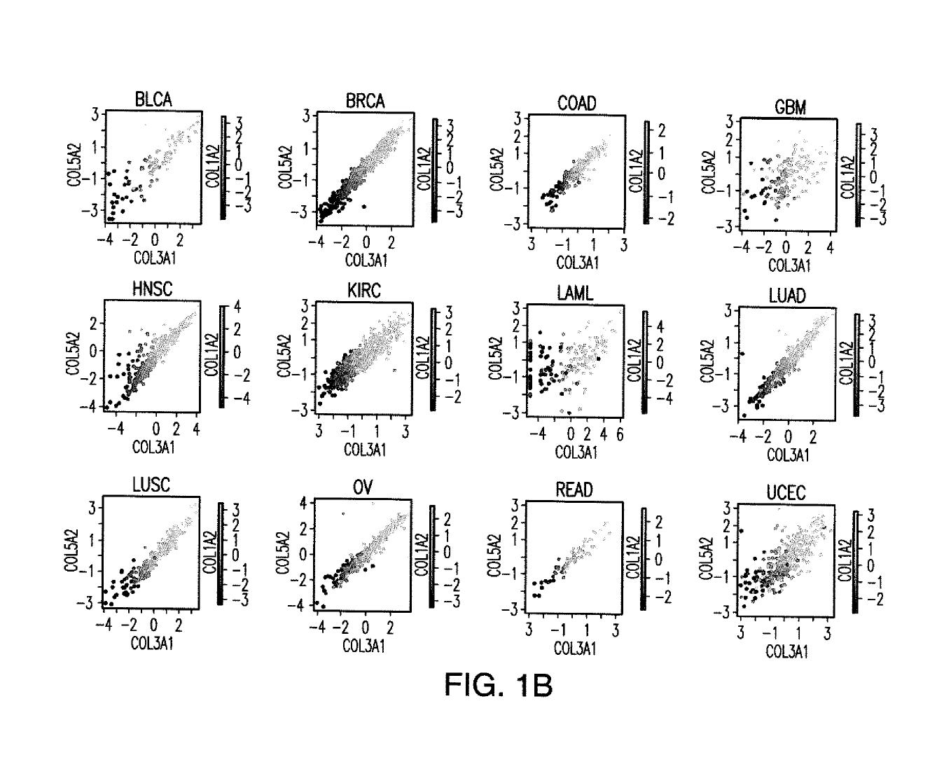

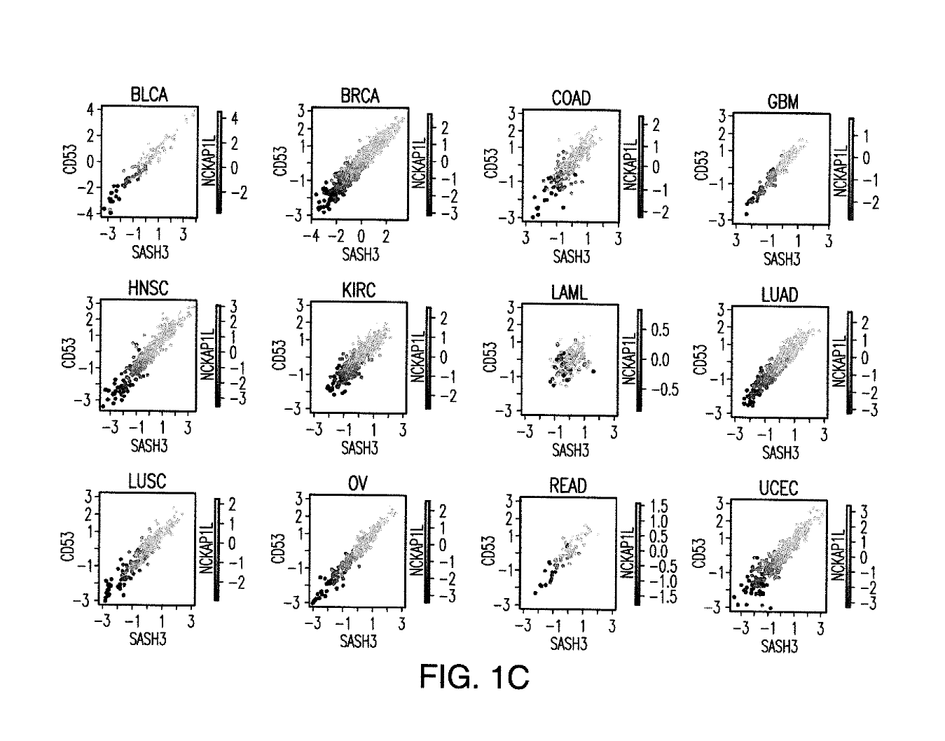

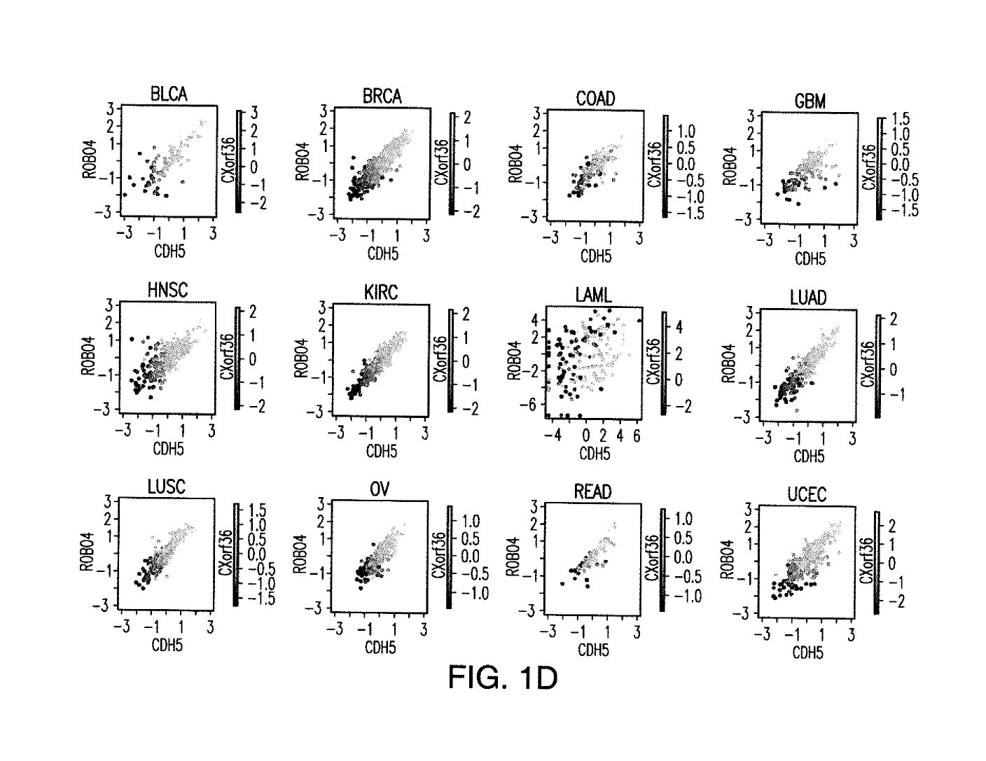

A striking visualization consistent with the co-expression of these pan-cancer molecular signatures can be made in the form of scatter plots. For example, FIG. 1 shows such color-coded scatter plots for the four main attractor metagenes CIN, MES, LYM, and END, in all twelve cancer types using the three top-ranked genes for each of these four metagenes. In each scatter plot, samples represented by dots at the lower left (blue) side have low levels of the signature, while samples represented by dots at the upper right (red) side have high levels of the signature. FIGS. 3-17 show the corresponding scatter plots for all 15 identified attractor molecular signatures demonstrating such coexpression in all cases.

Scrutinizing each of these molecular signatures (such as a protein attractor that includes cleaved PARP, Caspase-8, c-Met and Snail) provides opportunities for biological discovery. For example, the top-ranked genes of the END attractor metagene are CDH5, ROBO4, CXorf36, CD34, CLEC14A, ARHGEF, CD93, CLEC14A, ARHGEF15, CD93, LDB2, ELTD, MYCT1. Nearly all these genes are endothelial markers. The top gene, CDH5, codes for VE-cadherin, which is known to be involved in a pathway suppressing angiogenic sprouting (Abraham, S. et al. Curr Biol 19, 668-74 (2009)). The second gene, ROBO4, is known to inhibit VEGF-induced pathologic angiogenesis and endothelial hyperpermeability (Jones, C. A. et al. Nat Med 14, 448-53 (2008)). Consistently, the END attractor metagene appears to be protective and anti-angiogenic, stabilizing the vascular network. For example, 22 out of the 27 genes of the END attractor are among the 265 genes included in FIGS. 20A-D as most associated with patients' survival in a recent study (Wozniak, M. B. et al. PLoS One 8, e57886 (2013)) of renal cell carcinoma (P<8.4×10⁻³⁸ based on Fisher's exact test). These good-prognosis genes were intermixed in the same file with many poor-prognosis genes of the CIN attractor, suggesting that the CIN and END attractor metagenes are two of the most prognostic features in renal cell carcinoma.

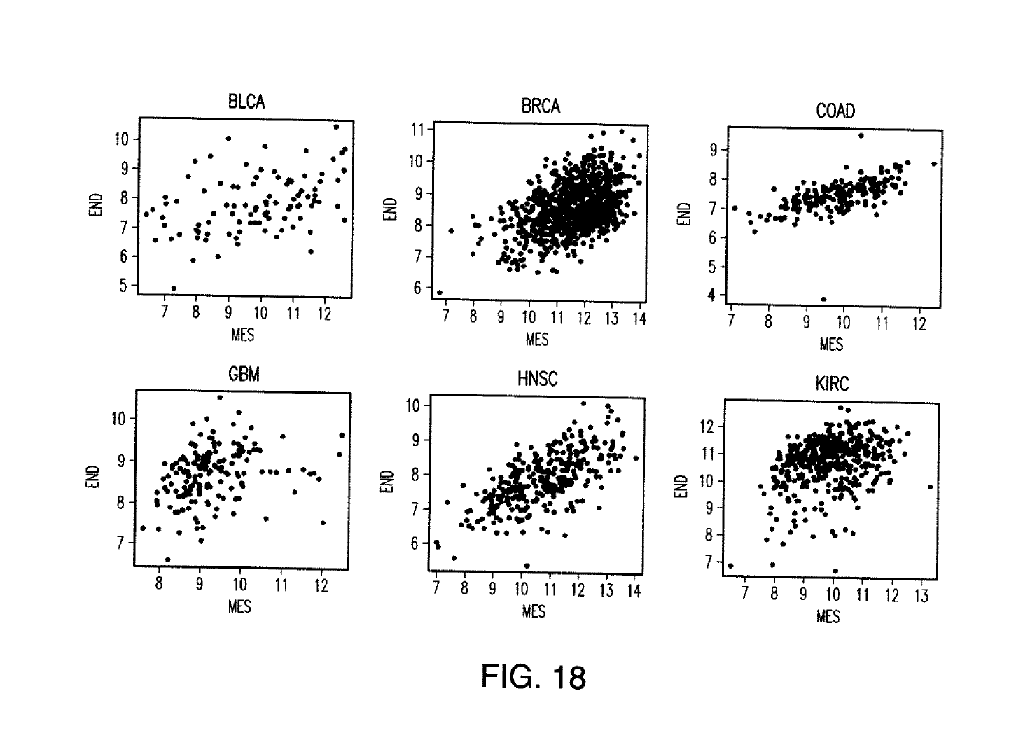

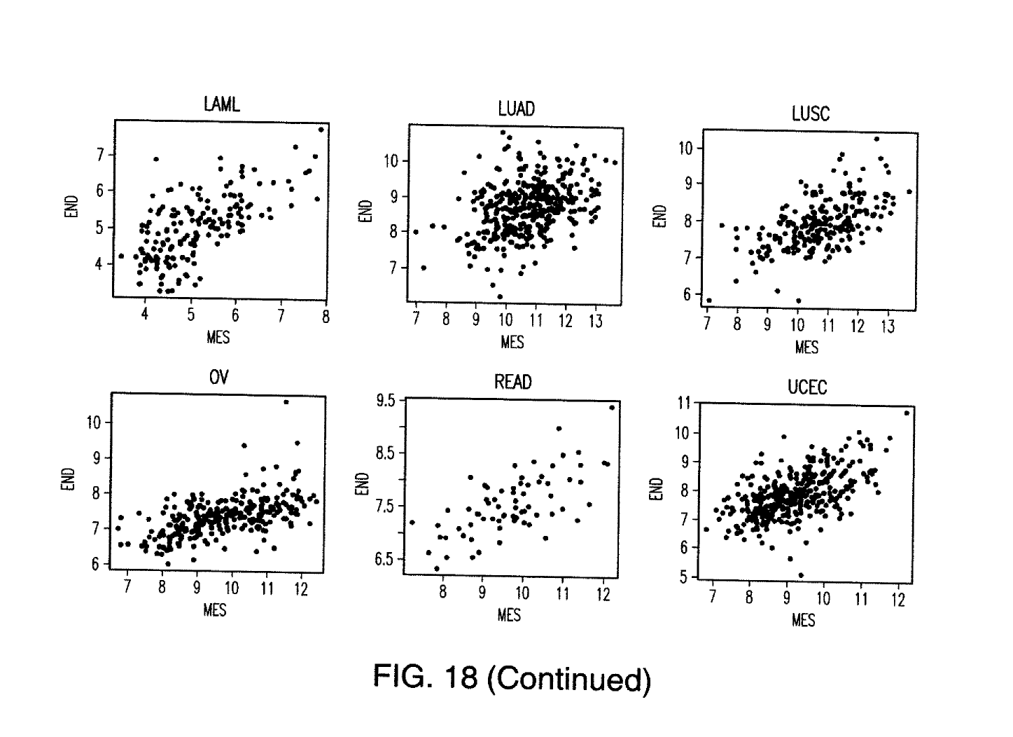

Interestingly, the MES and END attractor metagenes are positively associated with each other (FIG. 18), in the sense that overexpression of the END signature tends to imply overexpression of the MES signature and vice-versa. This is consistent with mutual exclusivity between angiogenesis and invasiveness and with related findings (Lu, K. V. et al. Cancer Cell 22, 21-35 (2012)) that VEGF inhibits tumor cell invasion and mesenchymal transition, while antiangiogenic therapy is associated with increased invasiveness (Paez-Ribes, M. et al. Cancer Cell 15, 220-31 (2009)). It may also explain the paradoxical protective nature of signatures related to the MES attractor metagene in invasive breast cancers (Beck, A. H., Espinosa, I., Gilks, C. B., van de Rijn, M. & West, R. B. Lab Invest 88, 591-601 (2008)), as the observed association of proteins such as SPARC with improved clinical outcome may be due the concomitant presence of the END signature. Indeed, SPARC, a key member of the MES signature, is also among the top 100 genes most associated with the END signature.

Two methylation attractor molecular signatures were observed to have a strong reverse association with each other, in the sense that the absence of one implied the strong presence of the other, or they were both present at intermediate levels. They were also found to be strongly associated with the lymphocyte-specific LYM attractor metagene. These two methylation signatures are referred to as M+ and M−, the former corresponding to hypermethylated sites in the presence of the LYM signature, and the latter corresponding to a hypomethylated site in the presence of the LYM signature. Six among the 27 genes of the M− signature (BIN2, TNFAIP8L2, ACAP1, NCKAP1L, FAM78A, PTPN7) are also among the 168 genes listed in the LYM attractor metagene (P<9.21×10⁻⁷ based on Fisher's exact test), suggesting that the LYM signature is at least partly triggered by the hypomethylation of the M− signature. FIG. 2 demonstrates, in the form of 12 scatter plots, this remarkable “methylation switch” and the association between LYM, M+ and M− signatures in all cancer types except leukemia. These results are consistent with previous findings (Andreopoulos, B. & Anastassiou, D. Cancer Inform 11, 61-75 (2012)) associating these signatures with the microRNA miR-142, but the current results indicate that this association of the LYM signature with M+ and M− appears to be strongly present in all solid cancer types. Given that the LYM signature is strongly protective in ER-negative breast cancers (Cheng, W. Y., Ou Yang, T. H. & Anastassiou, D. Sci Transl Med 5, 181ra50 (2013), further investigating the mechanisms behind these methylation signatures is a particularly promising area for further research.

The pan-cancer nature (FIGS. 3-17) of the 15 molecular signatures persented herein indicates that they represent important biomolecular events and offers the exciting opportunity that they can be used for diagnostic, predictive, and eventually therapeutic products, applicable in multiple cancers.

### 5.1 Example 2

### 5.2.1. Breast Cancer Prognostic Biomarker Comprising Attractor Metagenes and the FGD3-SUSD3 Metagene

Several prognostic models for breast cancer using molecular features have been used in biomarker products (see, e.g., Paik et al., N Engl J Med 2004; 351(27):2817-26; van't Veer et al., Nature 2002; 415(6871):530-6; and Parker et al., J Clin Oncol 2009; 27(8):1160-7), which have also proven to be of value to medical decision making, such as predicting whether an early-stage patient will benefit from adjuvant chemotherapy. A recent crowd-sourced research study, the Sage Bionetworks-DREAM Breast Cancer Prognosis Challenge (BCC) (Margolin et al., Sci Trans1 Med 2013; 5(181):181rel) used the METABRIC data set (Curtis et al., Nature 2012; 486(7403):346-52) containing molecular and clinical features from 1,981 breast cancer patients. The winning model (Cheng et al., Sci Transl Med 2013; 5(181): 181ra50 and McCarthy N., Nat Rev Cancer 2013; 13(6):378) as well as all five top-scoring models made use of several molecular features, called attractor metagenes (Cheng et al., PLoS Comput Biol 2013; 9(2):e1002920), as well as the FGD3-SUSD3 metagene defined by the average of the expression levels of the two genes, FGD3 and SUSD3, which are located directly adjacent to each other at Chr9q22.31.

To make a prognostic tool useable in a clinical setting derived from such metagenes, a new model based on the disease-specific survival information included in the METABRIC data set was prepared, providing an estimate of the breast cancer specific 10-year survival rate for each patient. This prognostic tool is referred to herein as the BCAM (Breast Cancer Attractor Metagenes) biomarker. The model was derived using the uniformly renormalized 1,981-sample METABRIC data set (Margolin et al., Sci Transl Med 2013; 5(181):181rel). As disclosed herein, the two genes whose high expression is most associated with good prognosis are FGD3 and SUSD3. At the other extreme, the genes whose high expression is most associated with poor prognosis were members of the mitotic chromosomal instability (“CIN”) attractor metagene, which was previously identified as a “pan-cancer” molecular signature using unsupervised analysis of other data sets from different cancer types (Cheng et al., PLoS Comput Biol 2013; 9(2):e1002920).

### 5.2.2. Methods

### 5.2.2.1. Data Sets, Pre-Processing, End Points of Survival Analysis

Because most breast cancer data sets do not include the number of positive lymph nodes, the requirements for acceptable validation data sets were relaxed to allow for those that merely provide a binary (negative/positive) lymph node status. Still, only four data sets were found (Table 1) in addition to METABRIC, with the requirements that they include probes for genes FGD3 and SUSD3, tumor size, lymph node status and disease-specific survival or recurrence data, from which at least one statistically significant (P<0.05) comparison between the BCAM formula and those used in other genomic assays could be extracted. Only the Buffa data set provides the number of positive lymph nodes; in the other data sets the BCAM formula setting the number of positive lymph nodes for lymph node positive patients to 1 was used. The tumor size and the lymph node number were logarithmically transformed.

### Table 1

| | | Accession | |
|---|---|---|---|
| Data set | Source | Number | Reference |
| METABRIC | Sage Synapse | syn1710250 | [5] |
| Loi | GEO | GSE6532 | [17] |
| Buffa | GEO | GSE22219 | [18] |
| Wang | GEO | GSE19615 | [19] |
| Miller | GEO | GSE3494 | [20] |

The data sets generated from Affymetrix U133A/B, and Plus2.0 arrays were renormalized using Robust Multi-array Average (RMA), as implemented in the Affy package in Bioconductor (www.bioconductor.org) in the R software. If there was more than one platform provided for each patient, the measurements were combined and renormalized using RMA. The METABRIC data set was renormalized by Sage Synapse (Margolin et al., Sci Transl Med 2013; 5(181):181rel). Because the BCAM formula is the linear combination of heterogeneous covariates, the distribution of genomic assays in each data set were corrected by multiplying the size and the lymph node number with the ratio of the standard-deviations of the genomic assays in each data set to the standard-deviation of the genomic assays in the METABRIC data set.

For survival analysis, because each data set uses different end point for censoring, the end point defined closest to disease-specific survival available in the METABRIC data set and in the Miller data set were used. Time to recurrence in the Loi and Wang data sets and distant-relapse free survival in the Buffa data set were used.

### 5.2.2.2. Comparison of Predictive Models

The concordance index (Pencina et al., Stat Med 2004; 23(13):2109-23) was used to assess the accuracy of the rankings of patients' risk. It is defined as the relative frequency of accurate pairwise predictions of survival ranking over all pairs of patients for which such a determination can be achieved. To compare the performances of the predictive models, the distribution of the concordance index were estimated as the overall C-index for each model on each subset of samples. Since the overall C estimator is proven to be asymptotically normal, the null distribution of the C-index can be approximated by a normal distribution with mean 0.5 and the sampling variance of C-index when the sample size is sufficiently large. Standardized by the mean under the null hypothesis and estimated variance from data, the C-index follows a Student's t distribution approximately. The difference between two estimated C-indices, after standardization, also follows a t distribution approximately under the null hypothesis that the two C-indices are equal. Therefore, the comparison between two overall C-indices can be carried out by a Student's t-test and the P value is evaluated accordingly. The overall C-index estimation and t-test were performed by the survcomp package (Schroder et al., Bioinformatics 2011; 27(22):3206-8) in the R software.

### 5.2.2.3. Feature Selector Facility

The prognostic score displayed for each combination of selected features was designed to be resistant to overfitting. It is evaluated as the asymptotic average of the concordance indices resulting from random 2-fold cross-validation experiments in the METABRIC data set. Each experiment uses the selected features as covariates to train a Cox proportional hazards model on half of the data set based on random splitting, and evaluates the corresponding concordance index of the fitted model on the other half. Each experiment is also repeated by reversing the training/validation roles of the same subsets.

### 5.2.2.4. Estimation of Survival Rate

The final BCAM score between 0 and 100 is generated as the corresponding percentile value from the Cox model formula against the 1,981-sample METABRIC data set. The breast cancer specific 10-year survival rate associated with the BCAM score is found by calculating the Kaplan-Meier hazard ratio at ten years for the METABRIC subpopulation inside a sliding window containing 20% of the samples (10% in each side) with the closest BCAM scores. If there are not enough patients on one side of the window, the window size was reduced so that it remains symmetric.

### 5.2.2.5. Other Breast Cancer Prognostic Formulas

BCAM was compared with four biomarkers used in other genomic assays: The 21-gene Oncotype DX signature, the 70-gene MammaPrint signature representing a good prognosis gene expression profile, the 50-gene ROR-S signature whose different expression profiles constitute centroids for four intrinsic PAM50 subtypes; and the ROR-C signature combining the PAM50 subtypes with original tumor size. The definition of each of the four groups in the 21-gene signature and the formula for combining them were obtained in (Paik et al., N Engl J Med 2004; 351(27):2817-26) without applying the cut-off thresholds, as the expression levels of the groups for the microarray values and RT-PCR values were not compatible. The score of the 70-gene assay was derived as described in the original papers (van't Veer et al., Nature 2002; 415(6871):530-6 and van de Vijver et al., N Engl J Med 2002; 347(25):1999-2009). The centroids of intrinsic subtypes were obtained from the Bioconductor package genefu. The formula of combining the individual scores for the four subtypes and tumor size were obtained from the original paper (Parker et al., J Clin Oncol 2009; 27(8):1160-7).

### 5.2.3. Results

### 5.2.3.1. Validation of the FGD3-SUSD3 Metagene

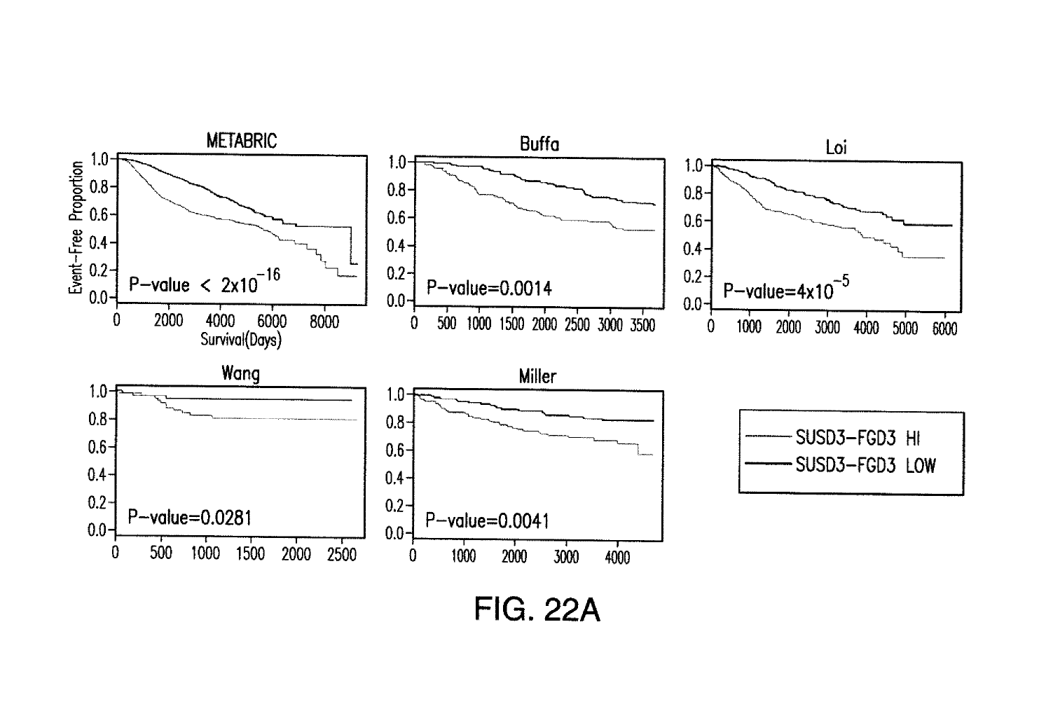

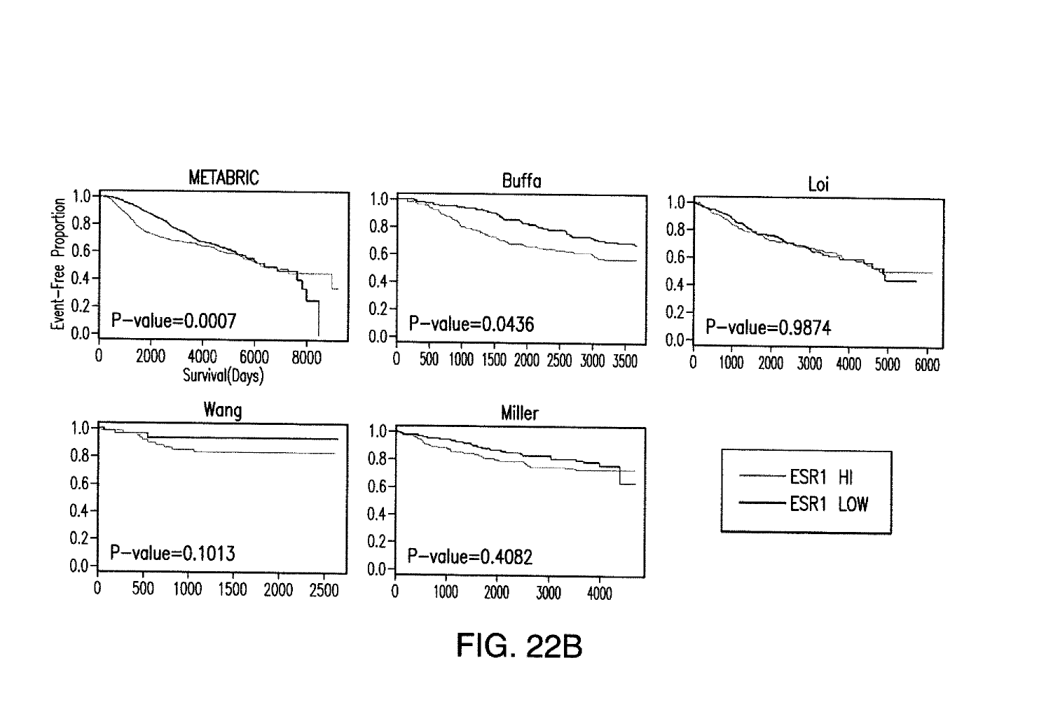

The breast cancer-specific FGD3-SUSD3 metagene, which was the most prognostic molecular feature in METABRIC, was first confirmed as highly prognostic in all other data sets. FIG. 22A shows the Kaplan-Meier survival curves of the FGD3-SUSD3 metagene demonstrating statistical significance in all five data sets. The gene most associated with the FGD3-SUSD3 metagene in METABRIC (also among the most associated ones in all the other data sets) is the estrogen receptor ESR1, which is less prognostic than FGD3-SUSD3 in all five data sets (FIG. 22B).

### 5.2.3.2. Feature Selection

The features of BCAM were selectied such that, when combined, they would enhance prognostic performance in the METABRIC data sets. The FGD3-SUSD3 metagene was included as a feature of BCAM. Additional metagenes, namely CIN (mitotic chromosomal instability), MES (mesenchymal transition) and LYM (lymphocyte infiltration) and two conditioned versions: MES*, restricted to early-stage tumors defined as lymph node negative with tumor size less than 30 mm, and LYM*, restricted to samples with more than three positive lymph nodes were also used (in the BCC it was found (Cheng et al., Sci Transl Med 2013; 5(181):181ra50) that MES was prognostic only in early-stage cancers and that LYM, though protective overall, was associated with poor prognosis in the presence of multiple positive lymph nodes). END, a multi-cancer molecular signature of endothelial markers was also employed. In addition, all the molecular features whose combination is used in existing breast cancer prognostic assays: Oncotype DX (proliferation, invasion, ER, HER2 groups, CD68, GSTM1, BAG1 genes); PAM50 defined molecular subtypes (Basal, Luminal A, Luminal B, HER2 features); the single 70-gene Mammaprint feature; and the three genes ESR1, PGR, ERBB2 used (Roepman et al., Clin Cancer Res 2009; 15(22):7003-11) in the TargetPrint assay were included. Finally, the number of positive lymph nodes and tumor size were also included as features.

A feature selection web-based facility (www.ee.columbia.edu/˜anastas/featureselector), was designed that evaluates a prognostic score after selecting a specified number among the above features. The score was designed so that it will cease increasing when overfitting has occurred. Logarithmic versions were included for the number of lymph nodes and the tumor size, because the score was found to become consistently higher if these versions were included rather than the direct values. The purpose of the overall facility is to provide an estimate of the performance of each of the existing assays by selecting the corresponding features, as well as to provide insight on the relative contribution of individual features when combined with other ones, leading to the selection of an optimal biomarker. Instructive results, noted in the facility, are the identified best selection of a given number N of features.

For N=1, the most prognostic feature among those listed in the facility is the “Luminal A” feature of PAM50, which measures the degree of correspondence with a good prognosis subtype. However, the Luminal A feature is eliminated from the best choice of features when N=2, in which case the optimal choice is the FGD3-SUSD3 metagene combined with the number of positive lymph nodes. At N=3 the CIN metagene is also selected, followed in increasing order by tumor size, MES*, LYM, LYM*, CD68 and END, each of which increases the score, at which point (N=9) it reaches the value of 0.741. Following this selection of nine features, no additional feature increases the score. To further increase performance, a heuristic optimization algorithm was employed by including randomly chosen single genes in combination with some or all of the selected features, retaining genes with known roles in cancer literature. Two additional genes, DNAJB9 and CXCL12, were thus identified for a total number of eleven features increasing the score to 0.747. DNAJB9 has the remarkable property that, if included among the potential features, is selected as early as N=4 (www.ee.columbia.edu/˜anastas/featureselector2). The other gene, CXCL12, is selected at N=7. Both of these genes are known to play important roles in cancer (Sterrenberg et al., Cancer Lett 2011; 312(2):129-42 and Boimel et al., Breast Cancer Res 2012; 14(1):R23).

### 5.2.3.3. BCAM Biomarker

The BCAM model was thus based on the Cox model formula (Table 2) defined by the full METABRIC data set using the eleven features FGD3-SUSD3, CIN, MES*, LYM, END, LYM*, CD68, DNAJB9, CXCL12, number of positive lymph nodes, and tumor size.

TABLE 2
Cox model formula for BCAM biomarker

| Features | Description | Coefficient |
|---|---|---|
| CIN | Average expression of CENPA, DLGAP5, MELK, | 0.2424 |
| | BUB1, KIF2C, KIF20A, KIF4A, CCNA2, CCNB2, | |
| | NCAPG | |
| MES* | Average expression of COL5A2, VCAN, SPARC, | 0.2676 |
| | THBS2, FBN1, COL1A2, COL5A1, FAP, AEBP1, | |
| | CTSK, restricted to node-negative patients with | |
| | tumor size less than 30 mm | |
| LYM | Average expression of PTPRC, CD53, LCP2, | −0.2868 |
| | LAPTM5, DOCK2, IL10RA, CYBB, CD48, | |
| | ITGB2, EVI2B | |
| LYM* | LYM restricted to patients with more than three | 0.5491 |
| | positive lymph nodes | |
| FGD3- | Average expression of FGD3 and SUSD3 | −0.2026 |
| SUSD3 | | |
| CD68 | CD68 gene | 0.1751 |
| TUMOR_ | Ln(Tumor size + 10) in mm | 0.5167 |
| SIZE | | |
| LYMPH# | Ln(Number of positive lymph nodes + 1) | 0.5563 |
| CXCL12 | CXCL12 gene | −0.2715 |
| DNAJB9 | DNAJB9 gene | −0.2914 |

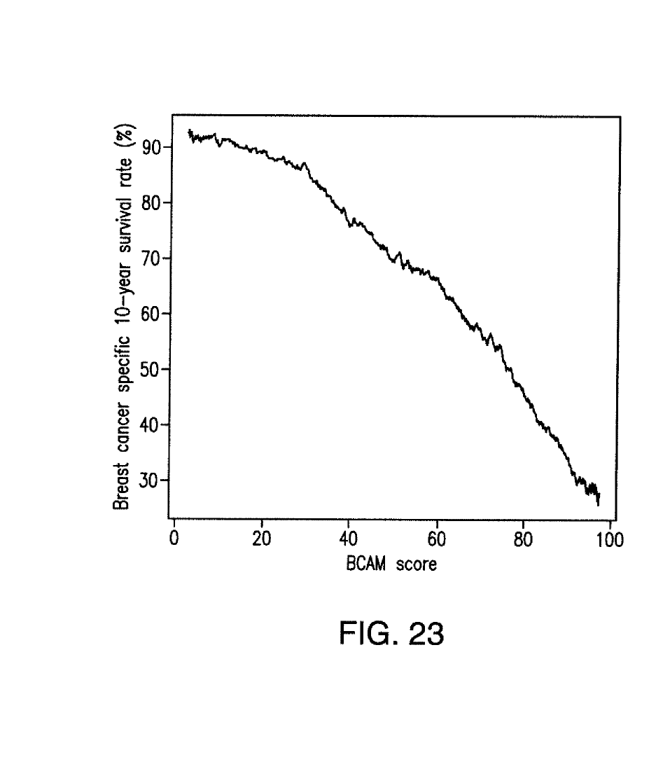

The final BCAM score between 0 and 100 is generated from the Cox model formula as the percentile value against the 1,981-sample METABRIC data set. FIG. 23 shows the estimated breast cancer specific 10-year survival rate as a function of the BCAM score.

### 5.2.3.4. Validation in Other Data Sets

The prognostic performance of the BCAM formula was compared with formulas of other genomic assays: Oncotype DX, Mammaprint, ROR-S (using PAM50 subtype information alone), and ROR-C (using PAM50 subtype information and tumor size). Other breast cancer data sets were deemed appropriate for evaluating prognostic values, which are refer to herein as: Loi (Loi et al., Proc Natl Acad Sci U S A 2010; 107(22):10208-13), Buffa (Buffa et al., Cancer Res 2011; 71(17):5635-45), Wang (Li et al., Nat Med 2010; 16(2):214-8) and Miller (Miller et al., Proc Natl Acad Sci U S A 2005; 102(38):13550-5). For each data set, the following two subsets were considered: 1) lymph node-negative (LNN) patients, and 2) estrogen receptor-positive (ERP) patients (regardless of PR and HER2 status). Additional intersection of these sets did not lead to results of statistical significance.

BCAM outperformed the other genomic assays in all cases in which comparisons had statistical significance (Table 3). In most of these comparisons (except when comparing BCAM with ROR-C in the LNN subsets), BCAM makes use of clinical information not used in the other assays. These results demonstrate the advantage of integrating clinical stage with molecular feature information into one product with enhanced prognostic power.

Table 3 includes a list of scores, measured by the corresponding concordance index, after applying the formula of each prognostic assay on cancer data sets and their lymph node-negative (LNN) and ER-positive (ERP) subsets. Shaded, but not bolded, are the values achieving highest score in each case. Shaded and in boldface are the scores for which the corresponding P value of comparison with the BCAM score is less than 0.05. The last set of rows contains the scores from the METABRIC data set and the listed BCAM scores result from applying the formula on the entire data set. These cannot be compared with other scores because METABRIC was used for BCAM training. ROR-S uses the gene expression based PAM50 assay; ROR-C uses the gene expression plus tumor size based PAM50 assay; 21-gene uses the Oncotype DX 21-gene assay; and 70-gene uses the Mammaprint 70-gene assay.

TABLE 3

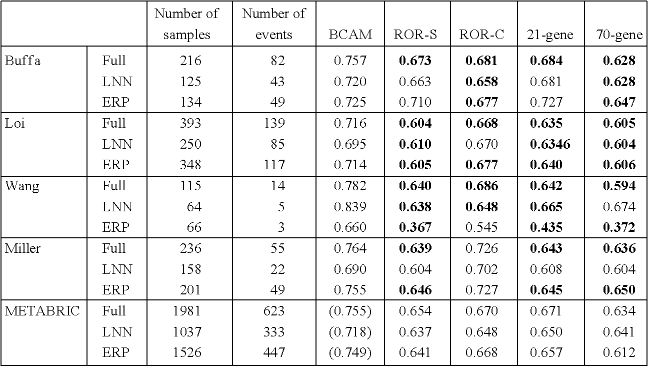

### 5.2.4.

### Discussion

The results of the analysis described herein lead to the unexpected and remarkable indication that breast cancer subtype classification, as well as estrogen/progesterone receptor and HER2 status do not provide any additional prognostic information in the presence of the expression levels of the FGD3-SUSD3 and the attractor metagenes. This indication is underscored by the fact that the uniformly renormalized 1981-sample METABRIC data set is uniquely rich and useful for reaching results of statistical significance in survival analysis.

In support of the above indication, using the web-based feature selector facility, for all feature combinations that were analyzed:
(a) Selecting the Oncotype DX Estrogen group, or any of genes ESR1 and PGR, in addition to any selected feature combination that includes metagenes FGD3-SUSD3 and CIN, does not increase, and in most cases decreases the score.
(b) Replacing the selection of the Oncotype DX Estrogen group or any of genes ESR1 and PGR (including any multiple selection of these features) with FGD3-SUSD3, in any selected feature combination, increases the score.

Many early versions of microarray platforms, notably the popular Affymetrix U133A, do not contain probes for FGD3 and SUSD3, which may provide some explanation as to why these genes were not found earlier as highly prognostic in breast cancer. The two genes are genomically adjacent to each other and are correlated with ESR1 and PGR. The simultaneous silencing of FGD3 and SUSD3 is strongly associated with poor prognosis. Furthermore, a recent study (Moy et al., Oncogene 2014; 10.1038/onc.2013.553) identified SUSD3 as the single most predictive gene (more than ESR1) of response to aromatase inhibitor therapy.

The alternative offered by the BCAM biomarker is one universal prognostic assay applicable to all breast cancer subtypes and stages, integrating tumor biology across stages. Indeed, as evidenced by the feature selector facility, the LYM and MES metagene would not be prognostic in the absence of stage information, and the conditioned LYM* and MES* features add significantly to the overall prognostic power. BCAM is also independent of tumor grade, since the CIN metagene is a proxy for, and more prognostic than, grade, or the expression of the Ki67 gene.

The inclusion of gene CD68, used in the Oncotype DX assay, was observed to improve the prognostic performance of the BCAM model. The expression of gene CD68, a marker of tumor associated macrophages, is associated with worse prognosis, although it is positively correlated with the protective LYM lymphocyte infiltration signature, and their combination improves prognostic ability.

Various patents, patent applications, and publications are cited herein, the contents of which are hereby incorporated by reference in their entireties.

What is claimed is:

1. A kit for detecting the presence of an attractor molecular signature comprising measuring means for one or more feature selected from the group consisting of the features associated with an attractor molecular signature of FIGS. 1-17, 19A-D, 20A-D, or 21A-D.

2. The kit of claim 1, wherein the attractor molecular signature is selected from the group consisting of: END; AHSA2; IFIT; WDR38; mir127; mir509; mir144; RMNDI; M+; M−; c-MET; and Akt attractor molecular signatures.

3. The kit of claim 2, wherein the one or more feature is selected from the genes of FIG. 19 associated with the corresponding attractor molecular signature.

4. The kit of claim 1, wherein the attractor molecular signature is the SCAM molecular attractor signature.

5. The kit of claim 4, comprising a measuring means for the following features: FGD3-SUSD3, CIN, MES*, LYM, END, LYM*, CD68, DNAJB9, and CXCL12.

6. A method of treatment wherein a patient sample is assayed for the presence of one or more feature selected from the group consisting of the genes associated with an attractor molecular signature of FIGS. 1-17, 19A-D, 20A-D, or 21A-D, and wherein, if said feature associated with the attractor molecular signature is present, thereafter adjusting said treatment accordingly.

7. The method of claim 6, wherein the attractor molecular signature is selected from the group consisting of: END; AHSA2; IFIT; WDR38; mir127; mir509; mir144; RMND1; M+; M−; c-MET; and Akt attractor molecular signatures.

8. The method of claim 7, wherein the one or more feature is selected from the genes of FIG. 19 associated with the corresponding attractor molecular signature.

9. The method of claim 6, wherein a patient sample is assayed for the presence of the features FGD3-SUSD3, CIN, MES*, LYM, END, LYM*, CD68, DNAJB9, CXCL12, number of positive lymph nodes, and tumor size, and thereafter adjusting said treatment accordingly.

10. A method of performing a prognosis of a subject wherein a patient sample is assayed for the presence of one or more feature selected from the group consisting of the features associated with an attractor molecular signature of FIGS. 1-17, 19A-D, 20A-D, or 21A-D, and wherein, if said feature associated with the attractor molecular signature is present, predicting the likely outcome of the cancer.

11. The method of claim 10, wherein the attractor molecular signature is selected from the group consisting of: END; AHSA2; IFIT; WDR38; mir127; mir509; mir144; RMND1; M+; M−; c-MET; and Akt attractor molecular signatures.

12. The method of claim 11, wherein the one or more feature is selected from the genes of FIG. 19 associated with the corresponding attractor molecular signature.

13. The method of claim 10, wherein the patient sample is assayed for the presence of FGD3-SUSD3, CIN, MES*, LYM, END, LYM*, CD68, DNAJB9, CXCL12, number of positive lymph nodes, and tumor size and wherein, if said features are present, predicting the likely outcome of the cancer.
